(19) United States
(12) Patent Application Publication
(10) Pub. No.: US 2024/0294917 A1
(43) Pub. Date: Sep. 5, 2024

(54) CRISPR/CAS-RELATED METHODS AND COMPOSITIONS FOR TREATING HERPES SIMPLEX VIRUS TYPE 1 (HSV-1)

(71) Applicant: EDITAS MEDICINE, INC., CAMBRIDGE, MA (US)

(72) Inventors: Morgan L. Maeder, Jamaica Plain, MA (US); Ari E. Friedland, Boston, MA (US); G. Grant Welstead, Cambridge, MA (US); David A. Bumcrot, Belmont, MA (US)

(73) Assignee: EDITAS MEDICINE, INC, CAMBRIDGE, MA (US)

(21) Appl. No.: 18/452,233

(22) Filed: Aug. 18, 2023

Related U.S. Application Data
(63) Continuation of application No. 15/281,579, filed on Sep. 30, 2016, now abandoned.
Application No. 15/281,579 is a continuation of application No. PCT/US2015/023916, filed on Apr. 1, 2015.
(60) Provisional application No. 61/973,793, filed on Apr. 1, 2014.

(51) Int. Cl. C12N 15/113; C12N 15/63; C12N 15/85

(52) U.S. Cl. CPC C12N 15/1133; C12N 15/63; C12N 15/85; C12N 2310/10; C12N 2310/20; C12N 2310/3513; C12N 2800/80

(57) ABSTRACT

CRISPR/CAS-related compositions and methods for treatment of HSV-1 are disclosed.

### REFERENCE TO RELATED APPLICATIONS

This application is a continuation of U.S. patent application Ser. No. 15/281,579, filed on Sep. 30, 2016, which is a continuation of International Patent Application No. PCT/US2015/023916, filed on Apr. 1, 2015, which claims the benefit of U.S. Provisional Application No. 61/973,793, filed Apr. 1, 2014, the contents of each of which are hereby incorporated by reference in their entirety herein, and to each of which priority is claimed.

### SEQUENCE LISTING

The instant application contains a Sequence Listing which has been submitted in XML format electronically and is hereby incorporated by reference in its entirety. Said XML copy, created on Sep. 16, 2023, is named 084177_0280.xml and is 25,249,007 bytes in size. The Sequence Listing, electronically filed herewith, does not extend beyond the scope of the specification and thus does not contain new matter.

### FIELD OF THE INVENTION

The invention relates to CRISPR/CAS-related methods and components for editing of a target nucleic acid sequence, or modulating expression of a target nucleic acid sequence, and applications thereof in connection with herpes simplex virus type 1 (HSV-1).

### BACKGROUND

Herpes simplex virus type 1 (HSV-1) causes intermittent sores of the mouth and mucous membranes. It is a ubiquitous and highly contagious pathogen. The majority of the population develops the infection during childhood. By adulthood, up to 80% of the population in the United States is infected with HSV-1. New HSV-1 infections occur at a rate of 1.6 cases per 100 person years (Langenberg et al., 1999; New England Journal of Medicine 341:1432-1438). The most severe manifestations of HSV-1 infection include, e.g., keratitis, encephalitis, and meningitis.

Infection of HSV-1 is permanent. After initial infection with HSV-1, the virus establishes latent infection that lasts for the lifetime of the host. Initial infection with HSV-1 generally causes painful blistering of the mucous membranes of the lips and mouth or genital region. After initial infection, HSV-1 establishes latent infection in all subjects. Following establishment of latent infection, reactivation of HSV-1 can occur at any point during the lifetime of the subject. Reactivation of HSV-1 is more likely to occur in the elderly and in immunocompromised individuals, including in those who have cancer, those who have HIV/AIDs and in those who have undergone solid organ or hematopoietic stem cell transplant.

HSV-1 encephalitis and HSV-1 meningitis are among the most severe and debilitating types of HSV infections. HSV encephalitis is the most common form of non-epidemic encephalitis. The annual incidence of HSV encephalitis is 0.2-0.4 in 100,000 individuals (Saba et al., 2012; British Medical Journal 344: e3166). Subjects who develop HSV-1 encephalitis and/or meningitis commonly have permanent neurologic sequelae.

Ocular herpes can affect the epithelium of the eye, causing keratitis, or the retina, where it may lead to acute retinal necrosis. Keratitis is the most common form of ocular herpes. HSV-1 keratitis is the most common cause of infectious blindness in the developed world (Dawson et. al., Suvey of Ophthalmology 1976; 21(2): 121-135). Worldwide, there are approximately 1.5 million cases of HSV-related ophthalmologic disease and 40,000 cases of HSV-related blindness or severe monocular visual impairment annually (Krawczyk et. al., Public Library of Science One 2015; 10(1): e0116800. Farooq and Shukla 2012; Survey of Ophthalmology 57(5): 448-462.). HSV-1 retinitis most often affects adults and can cause acute retinal necrosis (ARN). ARN causes permanent visual damage in more than 50% of subjects (Roy et al., Ocular Immunology and Inflammation 2014; 22(3):170-174).

Newborns are a population at particular risk for developing severe HSV-1 infections. The disease is transmitted from the mother to the fetus during childbirth. The chance of maternal-fetal transmission is highest in cases where the mother developed primary HSV infection during pregnancy. The incidence of neonatal herpes is approximately 4-30 per 100,000 births (Brown Z A, et al., 2003; Journal of the American Medical Association; 289(2): 203-209. Dinh T-H, et al., 2008; Sexually Transmitted Disease; 35(1): 19-21). Neonates can develop severe HSV-1 keratitis, retinitis, encephalitis and/or meningitis. Neonatal ocular herpes can result in immediate, permanent vision loss. Ocular HSV-1 puts neonates at risk for later developing ARN. Untreated HSV-1 encephalitis leads to death in 50% of neonates. Even with prompt treatment with antiviral therapy, the majority of neonates who contract HSV-1 encephalitis or meningitis will suffer from permanent neurologic sequelae.

There are no curative or preventative treatments for HSV-1. Therapy is primarily given during acute infection. Primary HSV-1 infections can be treated with antiviral therapy, including acyclovir, valacyclovir and famciclovir. These therapies may reduce viral shedding, decrease pain and improve healing time of lesions. Re-activated, latent infections may resolve without treatment (may be self-limiting) or may be treated with anti-viral therapy. Antiviral therapy may be given prophylactically in certain situations, including during childbirth in a mother with a recent HSV-1 infection or reactivation.

Vaccines are in development for the prevention of HSV-1 infection. However, in controlled clinical trials, vaccination efficacy has been limited. A recent vaccine for both HSV-1 and HSV-2 infections was only 35% effective in preventing HSV-1 infection (Belshe et al., 2012; New England Journal of Medicine 366(1): 34-43).

Despite advances in antiretroviral therapies, there remains a need for the treatment and prevention of HSV-1 infection, particularly the treatment and prevention of HSV-1 associated keratitis, retinitis, encephalitis and meningitis. A therapy that can cure, prevent, or treat HSV-1 infections would be superior to the current standard of care.

### SUMMARY OF THE INVENTION

Methods and compositions discussed herein, provide for the treatment or prevention of herpes simplex virus type 1 (HSV-1), which causes intermittent sores of the mouth and mucous membranes. HSV-1 is contained within an icosahedral particle. The virus enters the host via infection of epithelial cells within the skin and mucous membranes. The virus produces immediate early genes within the epithelial cells, which encode enzymes and binding proteins necessary for viral synthesis. After primary infection, the virus travels up sensory nerve axons via retrograde transport to the sensory dorsal root ganglion (DRG).

Within the DRG, it establishes a latent infection. The latent infection persists for the lifetime of the host. Within the DRG cell, the virus uncoats, viral DNA is transported into the nucleus, and key viral RNAs associated with latency are transcribed (including the LAT RNAs).

Methods and compositions discussed herein provide for treatment or prevention of herpes simplex virus type 1 (HSV-1), or its symptoms, e.g., by knocking out one or more of the HSV-1 viral genes, e.g., by knocking out one or more of UL19, UL30, UL48 and/or UL54 gene(s). In one aspect, methods and compositions discussed herein may be used to alter one or more of UL19, UL30, UL48 and/or UL54 gene(s) to treat or prevent HSV-1 by targeting the gene, e.g., the non-coding or coding regions, e.g., the promoter region, or a transcribed sequence, e.g., intronic or exonic sequence. In an embodiment, coding sequence, e.g., a coding region, e.g., an early coding region, of one or more of UL19, UL30, UL48 and/or UL54 gene(s), is targeted for alteration and knockout of expression.

In another aspect, the methods and compositions discussed herein may be used to alter one or more of UL19, UL30, UL48 and/or UL54 gene(s) to treat or prevent herpes simplex virus type 1 (HSV-1) by targeting the coding sequence of one or more of UL19, UL30, UL48 and/or UL54 gene(s). In one embodiment, the gene, e.g., the coding sequence of one or more of the UL19, UL30, UL48 and/or UL54 gene(s), are targeted to knockout one or more of UL19, UL30, UL48 and/or UL54 gene(s), e.g., to eliminate expression of one or more of UL19, UL30, UL48 and/or UL54 gene(s), e.g., to knockout one or more copies of one or more of UL19, UL30, UL48 and/or UL54 gene(s), e.g., by induction of an alteration comprising a deletion or mutation in one or more of UL19, UL30, UL48 and/or UL54 gene(s). In an embodiment, the method provides an alteration that comprises an insertion or deletion. As described herein, a targeted knockout approach is mediated by non-homologous end joining (NHEJ) using a CRISPR/Cas system comprising an enzymatically active Cas9 (eaCas9) molecule.

In one embodiment, an early coding sequence of one or more of UL19, UL30, UL48 and/or UL54 gene(s) are targeted to knockout one or more of UL19, UL30, UL48 and/or UL54 gene(s). In an embodiment, targeting affects one or more copies of the UL19, UL30, UL48 and/or UL54 gene(s). In an embodiment, a targeted knockout approach reduces or eliminates expression of one or more functional UL19, UL30, UL48 and/or UL54 gene product(s). In an embodiment, the method provides an alteration that comprises an insertion or deletion.

In another aspect, the methods and compositions discussed herein may be used to alter one or more of UL19, UL30, UL48 and/or UL54 gene(s) to treat or prevent HSV-1 by targeting non-coding sequence of the UL19, UL30, UL48 and/or UL54 gene(s), e.g., promoter, an enhancer, an intron, 3′UTR, and/or polyadenylation signal. In one embodiment, the gene(s), e.g., the non-coding sequence of one or more UL19, UL30, UL48 and/or UL54 gene(s), is targeted to knockout the gene(s), e.g., to eliminate expression of the gene(s), e.g., to knockout one or more copies of the UL19, UL30, UL48 and/or UL54 gene(s), e.g., by induction of an alteration comprising a deletion or mutation in the UL19, UL30, UL48 and/or UL54 gene(s). In an embodiment, the method provides an alteration that comprises an insertion or deletion.

“HSV-1 target UL19 position”, as used herein, refers to a position in the UL19 gene, which if altered by NHEJ-mediated alteration, results in reduction or elimination of expression of functional UL19 gene product. In an embodiment, the position is in the UL19 gene coding region, e.g., an early coding region.

“HSV-1 target UL30 position”, as used herein, refers to a position in the UL30 gene, which if altered by NHEJ-mediated alteration, results in reduction or elimination of expression of functional UL30 gene product. In an embodiment, the position is in the UL30 gene coding region, e.g., an early coding region.

“HSV-1 target UL48 position”, as used herein, refers to a position in the UL48 gene, which if altered by NHEJ-mediated alteration, results in reduction or elimination of expression of functional UL48 gene product. In an embodiment, the position is in the UL48 gene coding region, e.g., an early coding region.

“HSV-1 target UL54 position”, as used herein, refers to a position in the UL54 gene, which if altered by NHEJ-mediated alteration, results in reduction or elimination of expression of functional UL54 gene product. In an embodiment, the position is in the UL54 gene coding region, e.g., an early coding region.

“HSV-1 target position”, as used herein, refers to any of a HSV-1 target UL19 target position, a HSV-1 target UL30 target position, a HSV-1 target UL48 target position and/or a HSV-1 target UL54 target position.

In one aspect, disclosed herein is a gRNA molecule, e.g., an isolated or non-naturally occurring gRNA molecule, comprising a targeting domain which is complementary with a target domain from the UL19, UL30, UL48 or UL54 gene.

In an embodiment, the targeting domain of the gRNA molecule is configured to provide a cleavage event, e.g., a double strand break or a single strand break, sufficiently close to a HSV-1 target position in the UL19, UL30, UL48 or UL54 gene to allow alteration, e.g., alteration associated with NHEJ, of a HSV-1 target position in the UL19, UL30, UL48 or UL54 gene. In an embodiment, the targeting domain is configured such that a cleavage event, e.g., a double strand or single strand break, is positioned within 1, 2, 3, 4, 5, 10, 15, 20, 25, 30, 35, 40, 45, 50, 60, 70, 80, 90, 100, 150 or 200 nucleotides of a HSV-1 target position. The break, e.g., a double strand or single strand break, can be positioned upstream or downstream of a HSV-1 target position in the UL19, UL30, UL48 or UL54 gene. In an embodiment, the targeting domain of the gRNA molecule is configured to provide a cleavage event selected from a double strand break and a single strand break, within 500 (e.g., within 500, 400, 300, 250, 200, 150, 100, 80, 60, 40, 20, or 10) nucleotides of a HSV-1 target position.

In an embodiment, a second gRNA molecule comprising a second targeting domain is configured to provide a cleavage event, e.g., a double strand break or a single strand break, sufficiently close to the HSV-1 target position in the UL19, UL30, UL48 or UL54 gene, to allow alteration, e.g., alteration associated with NHEJ, of the HSV-1 target position in the UL19, UL30, UL48 or UL54 gene, either alone or in combination with the break positioned by said first gRNA molecule. In an embodiment, the targeting domains of the first and second gRNA molecules are configured such that a cleavage event, e.g., a double strand or single strand break, is positioned, independently for each of the gRNA molecules, within 1, 2, 3, 4, 5, 10, 15, 20, 25, 30, 35, 40, 45, 50, 60, 70, 80, 90, 100, 150 or 200 nucleotides of the target position. In an embodiment, the breaks, e.g., double strand or single strand breaks, are positioned on both sides of a nucleotide of a HSV-1 target position in the UL19, UL30, UL48 or UL54 gene. In an embodiment, the breaks, e.g., double strand or single strand breaks, are positioned on one side, e.g., upstream or downstream, of a nucleotide of a HSV-1 target position in the UL19, UL30, UL48 or UL54 gene. In an embodiment, the targeting domain of the first and/or second gRNA molecule is configured to provide a cleavage event selected from a double strand break and a single strand break, within 500 (e.g., within 500, 400, 300, 250, 200, 150, 100, 80, 60, 40, 20, or 10) nucleotides of a HSV-1 target position.

In an embodiment, a single strand break is accompanied by an additional single strand break, positioned by a second gRNA molecule, as discussed below. For example, the targeting domains are configured such that a cleavage event, e.g., the two single strand breaks, are positioned within 1, 2, 3, 4, 5, 10, 15, 20, 25, 30, 35, 40, 45, 50, 60, 70, 80, 90, 100, 150 or 200 nucleotides of a HSV-1 target position. In an embodiment, the first and second gRNA molecules are configured such, that when guiding a Cas9 molecule, e.g., a Cas9 nickase, a single strand break will be accompanied by an additional single strand break, positioned by a second gRNA, sufficiently close to one another to result in alteration of a HSV-1 target position in the UL19, UL30, UL48 or UL54 gene. In an embodiment, the first and second gRNA molecules are configured such that a single strand break positioned by said second gRNA is within 10, 20, 30, 40, or 50 nucleotides of the break positioned by said first gRNA molecule, e.g., when the Cas9 molecule is a nickase. In an embodiment, the two gRNA molecules are configured to position cuts at the same position, or within a few nucleotides of one another, on different strands, e.g., essentially mimicking a double strand break.

In an embodiment, a double strand break can be accompanied by an additional double strand break, positioned by a second gRNA molecule, as is discussed below. For example, the targeting domain of a first gRNA molecule is configured such that a double strand break is positioned upstream of a HSV-1 target position in the UL19, UL30, UL48 or UL54 gene, e.g., within 1, 2, 3, 4, 5, 10, 15, 20, 25, 30, 35, 40, 45, 50, 60, 70, 80, 90, 100, 150 or 200 nucleotides of the target position; and the targeting domain of a second gRNA molecule is configured such that a double strand break is positioned downstream of a HSV-1 target position in the UL19, UL30, UL48 or UL54 gene, e.g., within 1, 2, 3, 4, 5, 10, 15, 20, 25, 30, 35, 40, 45, 50, 60, 70, 80, 90, 100, 150 or 200 nucleotides of the target position.

In an embodiment, a double strand break can be accompanied by two additional single strand breaks, positioned by a second gRNA molecule and a third gRNA molecule. For example, the targeting domain of a first gRNA molecule is configured such that a double strand break is positioned upstream of a HSV-1 target position in the UL19, UL30, UL48 or UL54 gene, e.g., within 1, 2, 3, 4, 5, 10, 15, 20, 25, 30, 35, 40, 45, 50, 60, 70, 80, 90, 100, 150 or 200 nucleotides of the target position; and the targeting domains of a second and third gRNA molecule are configured such that two single strand breaks are positioned downstream of a HSV-1 target position in the UL19, UL30, UL48 or UL54 gene, e.g., within 1, 2, 3, 4, 5, 10, 15, 20, 25, 30, 35, 40, 45, 50, 60, 70, 80, 90, 100, 150 or 200 nucleotides of the target position. In an embodiment, the targeting domain of the first, second and third gRNA molecules are configured such that a cleavage event, e.g., a double strand or single strand break, is positioned, independently for each of the gRNA molecules.

In an embodiment, a first and second single strand breaks can be accompanied by two additional single strand breaks positioned by a third gRNA molecule and a fourth gRNA molecule. For example, the targeting domain of a first and second gRNA molecule are configured such that two single strand breaks are positioned upstream of a HSV-1 target position in the UL19, UL30, UL48 or UL54 gene, e.g., within 1, 2, 3, 4, 5, 10, 15, 20, 25, 30, 35, 40, 45, 50, 60, 70, 80, 90, 100, 150 or 200 nucleotides of the target position; and the targeting domains of a third and fourth gRNA molecule are configured such that two single strand breaks are positioned downstream of a HSV-1 target position in the UL19, UL30, UL48 or UL54 gene, e.g., within 1, 2, 3, 4, 5, 10, 15, 20, 25, 30, 35, 40, 45, 50, 60, 70, 80, 90, 100, 150 or 200 nucleotides of the target position. In an embodiment, the targeting domain of the first, second, third, and/or fourth gRNA molecule is configured to provide a cleavage event selected from a double strand break and a single strand break, within 500 (e.g., within 500, 400, 300, 250, 200, 150, 100, 80, 60, 40, 20, or 10) nucleotides of a HSV-1 target position.

It is contemplated herein that, in an embodiment, when multiple gRNAs are used to generate (1) two single stranded breaks in close proximity, (2) two double stranded breaks, e.g., flanking a HSV-1 target position (e.g., to remove a piece of DNA, e.g., to create a deletion mutation) or to create more than one indel in the gene, e.g., in a coding region, e.g., an early coding region, (3) one double stranded break and two paired nicks flanking a HSV-1 target position (e.g., to remove a piece of DNA, e.g., to insert a deletion) or (4) four single stranded breaks, two on each side of a position, that they are targeting the same HSV-1 target position. It is further contemplated herein that multiple gRNAs may be used to target more than one HSV-1 target position in the same gene, e.g., one or more of UL19, UL30, UL48 and/or UL54 gene(s).

In an embodiment, the targeting domain of the first gRNA molecule and the targeting domain of the second gRNA molecules are complementary to opposite strands of the target nucleic acid molecule. In an embodiment, the gRNA molecule and the second gRNA molecule are configured such that the PAMs are oriented outward.

In an embodiment, the targeting domain of a gRNA molecule is configured to avoid unwanted target chromosome elements, such as repeat elements, e.g., Alu repeats, in the target domain. The gRNA molecule may be a first, second, third and/or fourth gRNA molecule, as described herein.

In an embodiment, the targeting domain of a gRNA molecule is configured to position a cleavage event sufficiently far from a preselected nucleotide, e.g., the nucleotide of a coding region, such that the nucleotide is not altered. In an embodiment, the targeting domain of a gRNA molecule is configured to position an intronic cleavage event sufficiently far from an intron/exon border, or naturally occurring splice signal, to avoid alteration of the exonic sequence or unwanted splicing events. The gRNA molecule may be a first, second, third and/or fourth gRNA molecule, as described herein.

In an embodiment, the targeting domain comprises a sequence that is the same as, or differs by no more than 1, 2, 3, 4, or 5 nucleotides from, a targeting domain sequence described herein, e.g., from any one of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27. In an embodiment, the targeting domain comprises a sequence that is the same as a targeting domain sequence described herein, e.g., from any one of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27.

In other embodiments, a HSV-1 target position in the coding region, e.g., the early coding region, of the UL19, UL30, UL48 or UL54 gene is targeted, e.g., for knockout. In an embodiment, the targeting domain comprises a sequence that is the same as, or differs by no more than 1, 2, 3, 4, or 5 nucleotides from, a targeting domain sequence from any one of Tables 1A-1G, Tables 2A-2G, Tables 3A-3G, or Tables 4A-4F.

In an embodiment, the targeting domain is independently selected from those in Tables 1A-1G. In an embodiment, the targeting domain is independently selected from Table 1A.

In an embodiment, the targeting domain is independently selected from those in Tables 2A-2G. In an embodiment, the targeting domain is independently selected from Table 2A.

In an embodiment, the targeting domain is independently selected from those in Tables 3A-3G. In an embodiment, the targeting domain is independently selected from Table 3A.

In an embodiment, the targeting domain is independently selected from those in Tables 4A-4F. In an embodiment, the targeting domain is independently selected from Table 4A.

In an embodiment, the targeting domain is independently selected from those in Tables 5A-5E. In an embodiment, the targeting domain is independently selected from Table 5A.

In an embodiment, the targeting domain is independently selected from those in Tables 6A-6G. In an embodiment, the targeting domain is independently selected from Table 6A.

In an embodiment, the targeting domain is independently selected from those in Tables 7A-7D. In an embodiment, the targeting domain is independently selected from Table 7A.

In an embodiment, the targeting domain is independently selected from those in Tables 8A-8E. In an embodiment, the targeting domain is independently selected from Table 8A.

In an embodiment, the targeting domain is independently selected from those in Tables 9A-9G. In an embodiment, the targeting domain is independently selected from Table 9A.

In an embodiment, the targeting domain is independently selected from those in Tables 10A-10C. In an embodiment, the targeting domain is independently selected from Table 10A.

In an embodiment, the targeting domain is independently selected from those in Tables 11A-11E. In an embodiment, the targeting domain is independently selected from Table 11A.

In an embodiment, the targeting domain is independently selected from those in Tables 12A-12G. In an embodiment, the targeting domain is independently selected from Table 12A.

In an embodiment, the targeting domain is independently selected from those in Tables 13A-13C. In an embodiment, the targeting domain is independently selected from Table 13A.

In an embodiment, the targeting domain is independently selected from those in Tables 14A-14E. In an embodiment, the targeting domain is independently selected from Table 14A.

In an embodiment, the targeting domain is independently selected from those in Tables 15A-15G. In an embodiment, the targeting domain is independently selected from Table 15A.

In an embodiment, the targeting domain is independently selected from those in Tables 16A-16C. In an embodiment, the targeting domain is independently selected from Table 16A.

In an embodiment, the targeting domain is independently selected from those in Table 27.

In an embodiment, when the HSV-1 target position is the UL19 gene coding region, e.g., an early coding region, and more than one gRNA is used to position breaks, e.g., two single stranded breaks or two double stranded breaks, or a combination of single strand and double strand breaks, e.g., to create one or more indels, in the target nucleic acid sequence, the targeting domain of each guide RNA is independently selected from any of Tables 1A-1G, Tables 5A-5E, Tables 6A-6G, or Tables 7A-7D.

In an embodiment, when the HSV-1 target position is the UL30 gene coding region, e.g., an early coding region, and more than one gRNA is used to position breaks, e.g., two single stranded breaks or two double stranded breaks, or a combination of single strand and double strand breaks, e.g., to create one or more indels, in the target nucleic acid sequence, the targeting domain of each guide RNA is independently selected from any of Tables 2A-2G, Tables 8A-E, Tables 9A-9G, or Tables 10A-10C.

In an embodiment, when the HSV-1 target position is the UL48 gene coding region, e.g., an early coding region, and more than one gRNA is used to position breaks, e.g., two single stranded breaks or two double stranded breaks, or a combination of single strand and double strand breaks, e.g., to create one or more indels, in the target nucleic acid sequence, the targeting domain of each guide RNA is independently selected from any of Tables 3A-3G, Tables 11A-11E, Tables 12A-12G, or Tables 13A-13C.

In an embodiment, when the HSV-1 target position is the UL54 gene coding region, e.g., an early coding region, and more than one gRNA is used to position breaks, e.g., two single stranded breaks or two double stranded breaks, or a combination of single strand and double strand breaks, e.g., to create one or more indels, in the target nucleic acid sequence, the targeting domain of each guide RNA is independently selected from any of Tables 4A-4F, Tables 14A-14E, Tables 15A-15G, or Tables 16A-16C.

In an embodiment, the gRNA, e.g., a gRNA comprising a targeting domain, which is complementary with the UL19, UL30, UL48 or UL54 gene, is a modular gRNA. In other embodiments, the gRNA is a unimolecular or chimeric gRNA.

In an embodiment, the targeting domain which is complementary with a target domain from the HSV-1 target position in the UL19, UL30, UL48 or UL54 gene is 16 nucleotides or more in length. In an embodiment, the targeting domain is 16 nucleotides in length. In an embodiment, the targeting domain is 17 nucleotides in length. In another embodiment, the targeting domain is 18 nucleotides in length. In still another embodiment, the targeting domain is 19 nucleotides in length. In still another embodiment, the targeting domain is 20 nucleotides in length. In still another embodiment, the targeting domain is 21 nucleotides in length. In still another embodiment, the targeting domain is 22 nucleotides in length. In still another embodiment, the targeting domain is 23 nucleotides in length. In still another embodiment, the targeting domain is 24 nucleotides in length. In still another embodiment, the targeting domain is 25 nucleotides in length. In still another embodiment, the targeting domain is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

A gRNA as described herein may comprise from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a gRNA comprises a linking domain of no more than 25 nucleotides in length; a proximal and tail domain, that taken together, are at least 20 nucleotides in length; and a targeting domain equal to or greater than 16, 17, 18, 19, 20, 21, 22, 23, 24, 25 or 26 nucleotides in length.

In another embodiment, a gRNA comprises a linking domain of no more than 25 nucleotides in length; a proximal and tail domain, that taken together, are at least 25 nucleotides in length; and a targeting domain equal to or greater than 16, 17, 18, 19, 20, 21, 22, 23, 24, 25 or 26 nucleotides in length.

In another embodiment, a gRNA comprises a linking domain of no more than 25 nucleotides in length; a proximal and tail domain, that taken together, are at least 30 nucleotides in length; and a targeting domain equal to or greater than 16, 17, 18, 19, 20, 21, 22, 23, 24, 25 or 26 nucleotides in length.

In another embodiment, a gRNA comprises a linking domain of no more than 25 nucleotides in length; a proximal and tail domain, that taken together, are at least 40 nucleotides in length; and a targeting domain equal to or greater than 16, 17, 18, 19, 20, 21, 22, 23, 24, 25 or 26 nucleotides in length.

A cleavage event, e.g., a double strand or single strand break, is generated by a Cas9 molecule. The Cas9 molecule may be an enzymatically active Cas9 (eaCas9) molecule, e.g., an eaCas9 molecule that forms a double strand break in a target nucleic acid or an eaCas9 molecule forms a single strand break in a target nucleic acid (e.g., a nickase molecule).

In an embodiment, the eaCas9 molecule catalyzes a double strand break.

In some embodiments, the eaCas9 molecule comprises HNH-like domain cleavage activity but has no, or no significant, N-terminal RuvC-like domain cleavage activity. In this case, the eaCas9 molecule is an HNH-like domain nickase, e.g., the eaCas9 molecule comprises a mutation at D10, e.g., D10A. In other embodiments, the eaCas9 molecule comprises N-terminal RuvC-like domain cleavage activity but has no, or no significant, HNH-like domain cleavage activity. In an embodiment, the eaCas9 molecule is an N-terminal RuvC-like domain nickase, e.g., the eaCas9 molecule comprises a mutation at H840, e.g., H840A. In an embodiment, the eaCas9 molecule is an N-terminal RuvC-like domain nickase, e.g., the eaCas9 molecule comprises a mutation at N863, e.g., N863A.

In an embodiment, a single strand break is formed in the strand of the target nucleic acid to which the targeting domain of said gRNA is complementary. In another embodiment, a single strand break is formed in the strand of the target nucleic acid other than the strand to which the targeting domain of said gRNA is complementary.

In another aspect, disclosed herein is a nucleic acid, e.g., an isolated or non-naturally occurring nucleic acid, e.g., DNA, that comprises (a) a sequence that encodes a gRNA molecule comprising a targeting domain that is complementary with a target domain, e.g., with a HSV-1 target position in UL19, UL30, UL48 or UL54 gene as disclosed herein.

In an embodiment, the nucleic acid encodes a gRNA molecule, e.g., a first gRNA molecule, comprising a targeting domain configured to provide a cleavage event, e.g., a double strand break or a single strand break, sufficiently close to a HSV-1 target position in the UL19, UL30, UL48 or UL54 gene to allow alteration, e.g., alteration associated with NHEJ, of a HSV-1 target position in the UL19, UL30, UL48 or UL54 gene.

In an embodiment, the nucleic acid encodes a gRNA molecule, e.g., the first gRNA molecule, comprising a targeting domain comprising a sequence that is the same as, or differs by no more than 1, 2, 3, 4, or 5 nucleotides from, a targeting domain sequence from any one of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27. In an embodiment, the nucleic acid encodes a gRNA molecule comprising a targeting domain is selected from those in Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27.

In an embodiment, the nucleic acid encodes a modular gRNA, e.g., one or more nucleic acids encode a modular gRNA. In another embodiment, the nucleic acid encodes a chimeric gRNA. The nucleic acid may encode a gRNA, e.g., the first gRNA molecule, comprising a targeting domain comprising 16 nucleotides or more in length. In an embodiment, the nucleic acid encodes a gRNA, e.g., the first gRNA molecule, comprising a targeting domain that is 16 nucleotides in length. In another embodiment, the nucleic acid encodes a gRNA, e.g., the first gRNA molecule, comprising a targeting domain that is 17 nucleotides in length. In yet another embodiment, the nucleic acid encodes a gRNA, e.g., the first gRNA molecule, comprising a targeting domain that is 18 nucleotides in length. In still another embodiment, the nucleic acid encodes a gRNA, e.g., the first gRNA molecule, comprising a targeting domain that is 19 nucleotides in length. In still another embodiment, the nucleic acid encodes a gRNA, e.g., the first gRNA molecule, comprising a targeting domain that is 20 nucleotides in length. In still another embodiment, the nucleic acid encodes a gRNA, e.g., the first gRNA molecule, comprising a targeting domain that is 21 nucleotides in length. In still another embodiment, the nucleic acid encodes a gRNA, e.g., the first gRNA molecule, comprising a targeting domain that is 22 nucleotides in length. In still another embodiment, the nucleic acid encodes a gRNA, e.g., the first gRNA molecule, comprising a targeting domain that is 23 nucleotides in length. In still another embodiment, the nucleic acid encodes a gRNA, e.g., the first gRNA molecule, comprising a targeting domain that is 24 nucleotides in length. In still another embodiment, the nucleic acid encodes a gRNA, e.g., the first gRNA molecule, comprising a targeting domain that is 25 nucleotides in length. In still another embodiment, the nucleic acid encodes a gRNA, e.g., the first gRNA molecule, comprising a targeting domain that is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

In an embodiment, a nucleic acid encodes a gRNA comprising from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a nucleic acid encodes a gRNA e.g., the first gRNA molecule, comprising a linking domain of no more than 25 nucleotides in length; a proximal and tail domain, that taken together, are at least 20 nucleotides in length; and a targeting domain equal to or greater than 16, 17, 18, 19, 20, 21, 22, 23, 24, 25 or 26 nucleotides in length.

In an embodiment, a nucleic acid encodes a gRNA e.g., the first gRNA molecule, comprising a linking domain of no more than 25 nucleotides in length; a proximal and tail domain, that taken together, are at least 25 nucleotides in length; and a targeting domain equal to or greater than 16, 17, 18, 19, 20, 21, 22, 23, 24, 25 or 26 nucleotides in length.

In an embodiment, a nucleic acid encodes a gRNA e.g., the first gRNA molecule, comprising a linking domain of no more than 25 nucleotides in length; a proximal and tail domain, that taken together, are at least 30 nucleotides in length; and a targeting domain equal to or greater than 16, 17, 18, 19, 20, 21, 22, 23, 24, 25 or 26 nucleotides in length.

In an embodiment, a nucleic acid encodes a gRNA comprising e.g., the first gRNA molecule, a linking domain of no more than 25 nucleotides in length; a proximal and tail domain, that taken together, are at least 40 nucleotides in length; and a targeting domain equal to or greater than 16, 17, 18, 19, 20, 21, 22, 23, 24, 25 or 26 nucleotides in length.

In an embodiment, a nucleic acid comprises (a) a sequence that encodes a gRNA molecule, e.g., the first gRNA molecule, comprising a targeting domain that is complementary with a target domain in the UL19, UL30, UL48 or UL54 gene as disclosed herein, and further comprising (b) a sequence that encodes a Cas9 molecule.

The Cas9 molecule may be a nickase molecule, an enzymatically activating Cas9 (eaCas9) molecule, e.g., an eaCas9 molecule that forms a double strand break in a target nucleic acid and/or an eaCas9 molecule that forms a single strand break in a target nucleic acid. In an embodiment, a single strand break is formed in the strand of the target nucleic acid to which the targeting domain of said gRNA is complementary. In another embodiment, a single strand break is formed in the strand of the target nucleic acid other than the strand to which to which the targeting domain of said gRNA is complementary.

In an embodiment, the eaCas9 molecule catalyzes a double strand break.

In an embodiment, the eaCas9 molecule comprises HNH-like domain cleavage activity but has no, or no significant, N-terminal RuvC-like domain cleavage activity. In another embodiment, the said eaCas9 molecule is an HNH-like domain nickase, e.g., the eaCas9 molecule comprises a mutation at D10, e.g., D10A. In another embodiment, the eaCas9 molecule comprises N-terminal RuvC-like domain cleavage activity but has no, or no significant, HNH-like domain cleavage activity. In another embodiment, the eaCas9 molecule is an N-terminal RuvC-like domain nickase, e.g., the eaCas9 molecule comprises a mutation at H840, e.g., H840A. In another embodiment, the eaCas9 molecule is an N-terminal RuvC-like domain nickase, e.g., the eaCas9 molecule comprises a mutation at N863, e.g., N863A.

A nucleic acid disclosed herein may comprise (a) a sequence that encodes a gRNA molecule comprising a targeting domain that is complementary with a target domain in the CCR5 gene as disclosed herein; (b) a sequence that encodes a Cas9 molecule.

A nucleic acid disclosed herein may comprise (a) a sequence that encodes a gRNA molecule comprising a targeting domain that is complementary with a target domain in the UL19, UL30, UL48 or UL54 gene as disclosed herein; (b) a sequence that encodes a Cas9 molecule; and further may comprise (c)(i) a sequence that encodes a second gRNA molecule described herein having a targeting domain that is complementary to a second target domain of the UL19, UL30, UL48 or UL54 gene, and optionally, (c)(ii) a sequence that encodes a third gRNA molecule described herein having a targeting domain that is complementary to a third target domain of the UL19, UL30, UL48 or UL54 gene; and optionally, (c)(iii) a sequence that encodes a fourth gRNA molecule described herein having a targeting domain that is complementary to a fourth target domain of the UL19, UL30, UL48 or UL54 gene.

In an embodiment, a nucleic acid encodes a second gRNA molecule comprising a targeting domain configured to provide a cleavage event, e.g., a double strand break or a single strand break, sufficiently close to a HSV-1 target position in the UL19, UL30, UL48 or UL54 gene, to allow alteration, e.g., alteration associated with NHEJ, of a HSV-1 target position in the UL19, UL30, UL48 or UL54 gene, either alone or in combination with the break positioned by said first gRNA molecule.

In an embodiment, a nucleic acid encodes a third gRNA molecule comprising a targeting domain configured to provide a cleavage event, e.g., a double strand break or a single strand break, sufficiently close to a HSV-1 target position in the UL19, UL30, UL48 or UL54 gene to allow alteration, e.g., alteration associated with NHEJ, of a HSV-1 target position in the UL19, UL30, UL48 or UL54 gene, either alone or in combination with the break positioned by the first and/or second gRNA molecule.

In an embodiment, a nucleic acid encodes a fourth gRNA molecule comprising a targeting domain configured to provide a cleavage event, e.g., a double strand break or a single strand break, sufficiently close to a HSV-1 target position in the UL19, UL30, UL48 or UL54 gene to allow alteration, e.g., alteration associated with NHEJ, of a HSV-1 target position in the UL19, UL30, UL48 or UL54 gene, either alone or in combination with the break positioned by the first gRNA molecule, the second gRNA molecule and/or the third gRNA molecule.

In an embodiment, the nucleic acid encodes a second gRNA molecule. The second gRNA is selected to target the same HSV-1 target position as the first gRNA molecule. Optionally, the nucleic acid may encode a third gRNA, and further optionally, the nucleic acid may encode a fourth gRNA molecule. The third gRNA molecule and the fourth gRNA molecule are selected to target the same HSV-1 target position as the first and second gRNA molecules.

In an embodiment, the nucleic acid encodes a second gRNA molecule comprising a targeting domain comprising a sequence that is the same as, or differs by no more than 1, 2, 3, 4, or 5 nucleotides from, a targeting domain sequence from one of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27. In an embodiment, the nucleic acid encodes a second gRNA molecule comprising a targeting domain selected from those in Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27. In an embodiment, when a third or fourth gRNA molecule are present, the third and fourth gRNA molecules may independently comprise a targeting domain comprising a sequence that is the same as, or differs by no more than 1, 2, 3, 4, or 5 nucleotides from, a targeting domain sequence from one of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27. In a further embodiment, when a third or fourth gRNA molecule are present, the third and fourth gRNA molecules may independently comprise a targeting domain selected from those in Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, Table 27.

In an embodiment, the nucleic acid encodes a second gRNA which is a modular gRNA, e.g., wherein one or more nucleic acid molecules encode a modular gRNA. In another embodiment, the nucleic acid encoding a second gRNA is a chimeric gRNA. In another embodiment, when a nucleic acid encodes a third or fourth gRNA, the third and fourth gRNA may be a modular gRNA or a chimeric gRNA. When multiple gRNAs are used, any combination of modular or chimeric gRNAs may be used.

A nucleic acid may encode a second, a third, and/or a fourth gRNA, each independently, comprising a targeting domain comprising 16 nucleotides or more in length. In an embodiment, the nucleic acid encodes a second gRNA comprising a targeting domain that is 16 nucleotides in length. In another embodiment, the nucleic acid encodes a second gRNA comprising a targeting domain that is 17 nucleotides in length. In another embodiment, the nucleic acid encodes a second gRNA comprising a targeting domain that is 18 nucleotides in length. In still another embodiment, the nucleic acid encodes a second gRNA comprising a targeting domain that is 19 nucleotides in length. In still another embodiment, the nucleic acid encodes a second gRNA comprising a targeting domain that is 20 nucleotides in length. In still another embodiment, the nucleic acid encodes a second gRNA comprising a targeting domain that is 21 nucleotides in length. In still another embodiment, the nucleic acid encodes a second gRNA comprising a targeting domain that is 22 nucleotides in length. In still another embodiment, the nucleic acid encodes a second gRNA comprising a targeting domain that is 23 nucleotides in length. In still another embodiment, the nucleic acid encodes a second gRNA comprising a targeting domain that is 24 nucleotides in length. In still another embodiment, the nucleic acid encodes a second gRNA comprising a targeting domain that is 25 nucleotides in length. In still another embodiment, the nucleic acid encodes a second gRNA comprising a targeting domain that is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

In an embodiment, a nucleic acid encodes a second, a third, and/or a fourth gRNA, each independently, comprising from 5′ to 3′: a targeting domain (comprising a “core domain”, and optionally a “secondary domain”); a first complementarity domain; a linking domain; a second complementarity domain; a proximal domain; and a tail domain. In some embodiments, the proximal domain and tail domain are taken together as a single domain.

In an embodiment, a nucleic acid encodes a second, a third, and/or a fourth gRNA comprising a linking domain of no more than 25 nucleotides in length; a proximal and tail domain, that taken together, are at least 20 nucleotides in length; and a targeting domain equal to or greater than 16, 17, 18, 19, 20, 21, 22, 23, 24, 25 or 26 nucleotides in length.

In an embodiment, a nucleic acid encodes a second, a third, and/or a fourth gRNA comprising a linking domain of no more than 25 nucleotides in length; a proximal and tail domain, that taken together, are at least 25 nucleotides in length; and a targeting domain equal to or greater than 16, 17, 18, 19, 20, 21, 22, 23, 24, 25 or 26 nucleotides in length.

In an embodiment, a nucleic acid encodes a second, a third, and/or a fourth gRNA comprising a linking domain of no more than 25 nucleotides in length; a proximal and tail domain, that taken together, are at least 30 nucleotides in length; and a targeting domain equal to or greater than 16, 17, 18, 19, 20, 21, 22, 23, 24, 25 or 26 nucleotides in length.

In an embodiment, a nucleic acid encodes a second, a third, and/or a fourth gRNA comprising a linking domain of no more than 25 nucleotides in length; a proximal and tail domain, that taken together, are at least 40 nucleotides in length; and a targeting domain equal to or greater than 16, 17, 18, 19, 20, 21, 22, 23, 24, 25 or 26 nucleotides in length.

In an embodiment, a nucleic acid encodes (a) a sequence that encodes a gRNA molecule comprising a targeting domain that is complementary with a target domain in the UL19, UL30, UL48 or UL54 gene, as disclosed herein, and (b) a sequence that encodes a Cas9 molecule, e.g., a Cas9 molecule described herein. In an embodiment, (a) and (b) are present on the same nucleic acid molecule, e.g., the same vector, e.g., the same viral vector, e.g., the same adeno-associated virus (AAV) vector. In an embodiment, the nucleic acid molecule is an AAV vector. Exemplary AAV vectors that may be used in any of the described compositions and methods include an AAV2 vector, a modified AAV2 vector, an AAV3 vector, a modified AAV3 vector, an AAV6 vector, a modified AAV6 vector, an AAV8 vector and an AAV9 vector.

In another embodiment, (a) is present on a first nucleic acid molecule, e.g. a first vector, e.g., a first viral vector, e.g., a first AAV vector; and (b) is present on a second nucleic acid molecule, e.g., a second vector, e.g., a second vector, e.g., a second AAV vector. The first and second nucleic acid molecules may be AAV vectors.

In another embodiment, a nucleic acid encodes (a) a sequence that encodes a gRNA molecule comprising a targeting domain that is complementary with a target domain in the UL19, UL30, UL48 or UL54 gene as disclosed herein, and (b) a sequence that encodes a Cas9 molecule, e.g., a Cas9 molecule described herein; and further comprises (c)(i) a sequence that encodes a second gRNA molecule as described herein, and optionally (c)(ii) a sequence that encodes a third gRNA molecule described herein having a targeting domain that is complementary to a third target domain of the UL19, UL30, UL48 or UL54 gene; and optionally, (c)(iii) a sequence that encodes a fourth gRNA molecule described herein having a targeting domain that is complementary to a fourth target domain of the UL19, UL30, UL48 or UL54 gene. In an embodiment, the nucleic acid comprises (a), (b) and (c)(i). In an embodiment, the nucleic acid comprises (a), (b), (c)(i) and (c)(ii). In an embodiment, the nucleic acid comprises (a), (b), (c)(i), (c)(ii) and (c)(iii). Each of (a) and (c)(i) may be present on the same nucleic acid molecule, e.g., the same vector, e.g., the same viral vector, e.g., the same adeno-associated virus (AAV) vector. In an embodiment, the nucleic acid molecule is an AAV vector.

In another embodiment, (a) and (c)(i) are on different vectors. For example, (a) may be present on a first nucleic acid molecule, e.g. a first vector, e.g., a first viral vector, e.g., a first AAV vector; and (c)(i) may be present on a second nucleic acid molecule, e.g., a second vector, e.g., a second vector, e.g., a second AAV vector. In an embodiment, the first and second nucleic acid molecules are AAV vectors.

In another embodiment, each of (a), (b), and (c)(i) are present on the same nucleic acid molecule, e.g., the same vector, e.g., the same viral vector, e.g., an AAV vector. In an embodiment, the nucleic acid molecule is an AAV vector. In an alternate embodiment, one of (a), (b), and (c)(i) is encoded on a first nucleic acid molecule, e.g., a first vector, e.g., a first viral vector, e.g., a first AAV vector; and a second and third of (a), (b), and (c)(i) is encoded on a second nucleic acid molecule, e.g., a second vector, e.g., a second vector, e.g., a second AAV vector. The first and second nucleic acid molecule may be AAV vectors.

In an embodiment, (a) is present on a first nucleic acid molecule, e.g., a first vector, e.g., a first viral vector, a first AAV vector; and (b) and (c)(i) are present on a second nucleic acid molecule, e.g., a second vector, e.g., a second vector, e.g., a second AAV vector. The first and second nucleic acid molecule may be AAV vectors.

In another embodiment, (b) is present on a first nucleic acid molecule, e.g., a first vector, e.g., a first viral vector, e.g., a first AAV vector; and (a) and (c)(i) are present on a second nucleic acid molecule, e.g., a second vector, e.g., a second vector, e.g., a second AAV vector. The first and second nucleic acid molecule may be AAV vectors.

In another embodiment, (c)(i) is present on a first nucleic acid molecule, e.g., a first vector, e.g., a first viral vector, e.g., a first AAV vector; and (b) and (a) are present on a second nucleic acid molecule, e.g., a second vector, e.g., a second vector, e.g., a second AAV vector. The first and second nucleic acid molecule may be AAV vectors.

In another embodiment, each of (a), (b) and (c)(i) are present on different nucleic acid molecules, e.g., different vectors, e.g., different viral vectors, e.g., different AAV vector. For example, (a) may be on a first nucleic acid molecule, (b) on a second nucleic acid molecule, and (c)(i) on a third nucleic acid molecule. The first, second and third nucleic acid molecule may be AAV vectors.

In another embodiment, when a third and/or fourth gRNA molecule are present, each of (a), (b), (c)(i), (c)(ii) and (c)(iii) may be present on the same nucleic acid molecule, e.g., the same vector, e.g., the same viral vector, e.g., an AAV vector. In an embodiment, the nucleic acid molecule is an AAV vector. In an alternate embodiment, each of (a), (b), (c)(i), (c)(ii) and (c)(iii) may be present on the different nucleic acid molecules, e.g., different vectors, e.g., the different viral vectors, e.g., different AAV vectors. In a further embodiment, each of (a), (b), (c)(i), (c) (ii) and (c)(iii) may be present on more than one nucleic acid molecule, but fewer than five nucleic acid molecules, e.g., AAV vectors.

The nucleic acids described herein may comprise a promoter operably linked to the sequence that encodes the gRNA molecule of (a), e.g., a promoter described herein. The nucleic acid may further comprise a second promoter operably linked to the sequence that encodes the second, third and/or fourth gRNA molecule of (c), e.g., a promoter described herein. The promoter and second promoter differ from one another. In some embodiments, the promoter and second promoter are the same.

The nucleic acids described herein may further comprise a promoter operably linked to the sequence that encodes the Cas9 molecule of (b), e.g., a promoter described herein.

In another aspect, disclosed herein is a composition comprising (a) a gRNA molecule comprising a targeting domain that is complementary with a target domain in the UL19, UL30, UL48 or UL54 gene, as described herein. The composition of (a) may further comprise (b) a Cas9 molecule, e.g., a Cas9 molecule as described herein. A composition of (a) and (b) may further comprise (c) a second, third and/or fourth gRNA molecule, e.g., a second, third and/or fourth gRNA molecule described herein. In an embodiment, the composition is a pharmaceutical composition. The compositions described herein, e.g., pharmaceutical compositions described herein, can be used in the treatment or prevention of HSV-1 in a subject, e.g., in accordance with a method disclosed herein.

In another aspect, disclosed herein is a method of altering a cell, e.g., altering the structure, e.g., altering the sequence, of a target nucleic acid of a cell, comprising contacting said cell with: (a) a gRNA that targets the UL19, UL30, UL48 or UL54 gene, e.g., a gRNA as described herein; (b) a Cas9 molecule, e.g., a Cas9 molecule as described herein; and optionally, (c) a second, third and/or fourth gRNA that targets UL19, UL30, UL48 or UL54 gene, e.g., a second, third and/or fourth gRNA, as described herein.

In an embodiment, the method comprises contacting said cell with (a) and (b).

In an embodiment, the method comprises contacting said cell with (a), (b), and (c).

The targeting domain of the gRNA of (a) and optionally (c) may be selected from any of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27, or a targeting domain of a gRNA that differs by no more than 1, 2, 3, 4, or 5 nucleotides from, a targeting domain sequence from any of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27.

In an embodiment, the method comprises contacting a cell from a subject suffering from or likely to develop HSV-1. The cell may be from a subject that would benefit from having a mutation at a HSV-1 target position.

In an embodiment, the contacting step may be performed in vivo.

In an embodiment, the method of altering a cell as described herein comprises acquiring knowledge of the sequence of a HSV-1 target position in said cell, prior to the contacting step. Acquiring knowledge of the sequence of a HSV-1 target position in the cell may be by sequencing one or more of the UL19, UL30, UL48 and/or UL54 gene, or a portion of the UL19, UL30, UL48 and/or UL54 gene.

In an embodiment, the contacting step of the method comprises contacting the cell with a nucleic acid, e.g., a vector, e.g., an AAV vector, that expresses at least one of (a), (b), and (c). In an embodiment, the contacting step of the method comprises contacting the cell with a nucleic acid, e.g., a vector, e.g., an AAV vector, that expresses each of (a), (b), and (c). In another embodiment, the contacting step of the method comprises delivering to the cell a Cas9 molecule of (b) and a nucleic acid which encodes a gRNA of (a) and optionally, a second gRNA (c)(i) and further optionally, a third gRNA (c)(ii) and/or fourth gRNA (c)(iii).

In an embodiment, the contacting step of the method comprises contacting the cell with a nucleic acid, e.g., a vector, e.g., an AAV vector, that expresses at least one of (a), (b), (c) and (d). In some embodiments, the contacting step of the method comprises contacting the cell with a nucleic acid, e.g., a vector, e.g., an AAV vector, that expresses each of (a), (b), and (c). In another embodiment, the contacting step of the method comprises delivering to the cell a Cas9 molecule of (b), a nucleic acid which encodes a gRNA of (a) and a template nucleic acid of (d), and optionally, a second gRNA (c)(i) and further optionally, a third gRNA (c)(ii) and/or fourth gRNA (c)(iii).

In an embodiment, contacting comprises contacting the cell with a nucleic acid, e.g., a vector, e.g., an AAV vector, e.g., an AAV2 vector, a modified AAV2 vector, an AAV3 vector, a modified AAV3 vector, an AAV6 vector, a modified AAV6 vector, an AAV8 vector or an AAV9 vector, as described herein.

In an embodiment, contacting comprises delivering to the cell a Cas9 molecule of (b), as a protein or an mRNA, and a nucleic acid which encodes a gRNA of (a) and optionally a second, third and/or fourth gRNA of (c).

In an embodiment, contacting comprises delivering to the cell a Cas9 molecule of (b), as a protein or an mRNA, said gRNA of (a), as an RNA, and optionally said second, third and/or fourth gRNA of (c), as an RNA.

In an embodiment, contacting comprises delivering to the cell a gRNA of (a) as an RNA, optionally the second, third and/or fourth gRNA of (c) as an RNA, and a nucleic acid that encodes the Cas9 molecule of (b).

In another aspect, disclosed herein is a method of treating a subject suffering from or likely to develop HSV-1, e.g., altering the structure, e.g., sequence, of a target nucleic acid of the subject, comprising contacting the subject (or a cell from the subject) with:
(a) a gRNA that targets the UL19, UL30, UL48 or UL54 gene, e.g., a gRNA disclosed herein;
(b) a Cas9 molecule, e.g., a Cas9 molecule disclosed herein; and optionally, (c)(i) a second gRNA that targets the UL19, UL30, UL48 or UL54 gene, e.g., a second gRNA disclosed herein, and further optionally, (c)(ii) a third gRNA, and still further optionally, (c)(iii) a fourth gRNA that target the UL19, UL30, UL48 or UL54 gene, e.g., a third and fourth gRNA disclosed herein.

In some embodiments, contacting comprises contacting with (a) and (b).

In some embodiments, contacting comprises contacting with (a), (b), and (c)(i).

In some embodiments, contacting comprises contacting with (a), (b), (c)(i) and (c)(ii).

In some embodiments, contacting comprises contacting with (a), (b), (c)(i), (c)(ii) and (c)(iii).

The targeting domain of the gRNA of (a) or (c) (e.g., (c)(i), (c)(ii), or (c)(iii)) may be selected from any of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27, or a targeting domain of a gRNA that differs by no more than 1, 2, 3, 4, or 5 nucleotides from, a targeting domain sequence from any of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27.

In an embodiment, the method comprises acquiring knowledge of the sequence at a HSV-1 target position in said subject.

In an embodiment, the method comprises acquiring knowledge of the sequence at a HSV-1 target position in said subject by sequencing one or more of the UL19, UL30, UL48 and/or UL54 gene(s) or a portion of the UL19, UL30, UL48 and/or UL54 gene.

In an embodiment, the method comprises introducing a mutation at a HSV-1 target position.

In an embodiment, the method comprises introducing a mutation at a HSV-1 target position by NHEJ.

In an embodiment, a cell of the subject is contacted is in vivo with (a), (b) and optionally (c)(i), further optionally (c)(ii), and still further optionally (c)(iii).

In an embodiment, the cell of the subject is contacted in vivo by intravenous delivery of (a), (b), and optionally (c)(i), further optionally (c)(ii), and still further optionally (c)(iii).

In an embodiment, the contacting step comprises contacting the subject with a nucleic acid, e.g., a vector, e.g., an AAV vector, described herein, e.g., a nucleic acid that encodes at least one of (a), (b), and optionally (c)(i), further optionally (c)(ii), and still further optionally (c)(iii).

In an embodiment, the contacting step comprises delivering to said subject said Cas9 molecule of (b), as a protein or mRNA, and a nucleic acid which encodes (a), and optionally (c)(i), further optionally (c)(ii), and still further optionally (c)(iii).

In an embodiment, the contacting step comprises delivering to the subject the Cas9 molecule of (b), as a protein or mRNA, the gRNA of (a), as an RNA, and optionally the second gRNA of (c)(i), further optionally said third gRNA of (c)(ii), and still further optionally said fourth gRNA of (c)(iii), as an RNA.

In an embodiment, the contacting step comprises delivering to the subject the gRNA of (a), as an RNA, optionally said second gRNA of (c)(i), further optionally said third gRNA of (c)(ii), and still further optionally said fourth gRNA of (c)(iii), as an RNA, a nucleic acid that encodes the Cas9 molecule of (b).

When the method comprises (1) introducing a mutation at a HSV-1 target position by NHEJ or (2) knocking down expression of one or more of the UL19, UL30, UL48 and/or UL54 gene(s), e.g., by targeting the promoter region, a Cas9 molecule of (b) and at least one guide RNA, e.g., a guide RNA of (a) are included in the contacting step.

In an embodiment, a cell of the subject is contacted is in vivo with (a), (b) and optionally (c)(i), further optionally (c)(ii), and still further optionally (c)(iii). In an embodiment, the cell of the subject is contacted in vivo by intravenous delivery of (a), (b) and optionally (c)(i), further optionally (c)(ii), and still further optionally (c)(iii).

In an embodiment, the contacting step comprises contacting the subject with a nucleic acid, e.g., a vector, e.g., an AAV vector, described herein, e.g., a nucleic acid that encodes at least one of (a), (b), and optionally (c)(i), further optionally (c)(ii), and still further optionally (c)(iii).

In an embodiment, the contacting step comprises delivering to said subject said Cas9 molecule of (b), as a protein or mRNA, and a nucleic acid which encodes (a) and optionally (c)(i), further optionally (c)(ii), and still further optionally (c)(iii).

In an embodiment, the contacting step comprises delivering to the subject the Cas9 molecule of (b), as a protein or mRNA, the gRNA of (a), as an RNA, and optionally the second gRNA of (c)(i), further optionally said third gRNA of (c)(ii), and still further optionally said fourth gRNA of (c)(iii), as an RNA.

In an embodiment, the contacting step comprises delivering to the subject the gRNA of (a), as an RNA, optionally said second gRNA of (c)(i), further optionally said third gRNA of (c)(ii), and still further optionally said fourth gRNA of (c)(iii), as an RNA, and a nucleic acid that encodes the Cas9 molecule of (b).

In another aspect, disclosed herein is a reaction mixture comprising a gRNA molecule, a nucleic acid, or a composition described herein, and a cell, e.g., a cell from a subject having, or likely to develop HSV-1, or a subject which would benefit from a mutation at a HSV-1 target position.

In another aspect, disclosed herein is a kit comprising, (a) a gRNA molecule described herein, or nucleic acid that encodes the gRNA, and one or more of the following:
(b) a Cas9 molecule, e.g., a Cas9 molecule described herein, or a nucleic acid or mRNA that encodes the Cas9;
(c)(i) a second gRNA molecule, e.g., a second gRNA molecule described herein or a nucleic acid that encodes (c)(i);
(c)(ii) a third gRNA molecule, e.g., a second gRNA molecule described herein or a nucleic acid that encodes (c)(ii);
(c)(iii) a fourth gRNA molecule, e.g., a second gRNA molecule described herein or a nucleic acid that encodes (c)(iii).

In an embodiment, the kit comprises nucleic acid, e.g., an AAV vector, that encodes one or more of (a), (b), (c)(i), (c)(ii), and (c)(iii).

In yet another aspect, disclosed herein is a gRNA molecule, e.g., a gRNA molecule described herein, for use in treating, or delaying the onset or progression of HSV-1 infection in a subject, e.g., in accordance with a method of treating, or delaying the onset or progression of HSV-1 infection as described herein.

In an embodiment, the gRNA molecule is used in combination with a Cas9 molecule, e.g., a Cas9 molecule described herein. Additionally or alternatively, in an embodiment, the gRNA molecule is used in combination with a second, third and/or fourth gRNA molecule, e.g., a second, third and/or fourth gRNA molecule described herein.

In still another aspect, disclosed herein is use of a gRNA molecule, e.g., a gRNA molecule described herein, in the manufacture of a medicament for treating, or delaying the onset or progression of HSV-1 in a subject, e.g., in accordance with a method of treating, or delaying the onset or progression of HSV-1 as described herein.

In an embodiment, the medicament comprises a Cas9 molecule, e.g., a Cas9 molecule described herein. Additionally or alternatively, in an embodiment, the medicament comprises a second, third and/or fourth gRNA molecule, e.g., a second, third and/or fourth gRNA molecule described herein.

The gRNA molecules and methods, as disclosed herein, can be used in combination with a governing gRNA molecule. As used herein, a governing gRNA molecule refers to a gRNA molecule comprising a targeting domain which is complementary to a target domain on a nucleic acid that encodes a component of the CRISPR/Cas system introduced into a cell or subject. For example, the methods described herein can further include contacting a cell or subject with a governing gRNA molecule or a nucleic acid encoding a governing molecule. In an embodiment, the governing gRNA molecule targets a nucleic acid that encodes a Cas9 molecule or a nucleic acid that encodes a target gene gRNA molecule. In an embodiment, the governing gRNA comprises a targeting domain that is complementary to a target domain in a sequence that encodes a Cas9 component, e.g., a Cas9 molecule or target gene gRNA molecule. In an embodiment, the target domain is designed with, or has, minimal homology to other nucleic acid sequences in the cell, e.g., to minimize off-target cleavage. For example, the targeting domain on the governing gRNA can be selected to reduce or minimize off-target effects. In an embodiment, a target domain for a governing gRNA can be disposed in the control or coding region of a Cas9 molecule or disposed between a control region and a transcribed region. In an embodiment, a target domain for a governing gRNA can be disposed in the control or coding region of a target gene gRNA molecule or disposed between a control region and a transcribed region for a target gene gRNA. While not wishing to be bound by theory, in an embodiment, it is believed that altering, e.g., inactivating, a nucleic acid that encodes a Cas9 molecule or a nucleic acid that encodes a target gene gRNA molecule can be effected by cleavage of the targeted nucleic acid sequence or by binding of a Cas9 molecule/governing gRNA molecule complex to the targeted nucleic acid sequence.

The compositions, reaction mixtures and kits, as disclosed herein, can also include a governing gRNA molecule, e.g., a governing gRNA molecule disclosed herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

Headings, including numeric and alphabetical headings and subheadings, are for organization and presentation and are not intended to be limiting.

Other features and advantages of the invention will be apparent from the detailed description, drawings, and from the claims.

### BRIEF DESCRIPTION OF THE DRAWINGS

FIGS. 1A-1I are representations of several exemplary gRNAs.

FIG. 1A depicts a modular gRNA molecule derived in part (or modeled on a sequence in part) from Streptococcus pyogenes (S. pyogenes) as a duplexed structure (SEQ ID NOS: 42 and 43, respectively, in order of appearance);

FIG. 1B depicts a unimolecular (or chimeric) gRNA molecule derived in part from S. pyogenes as a duplexed structure (SEQ ID NO: 44);

FIG. 1C depicts a unimolecular gRNA molecule derived in part from S. pyogenes as a duplexed structure (SEQ ID NO: 45);

FIG. 1D depicts a unimolecular gRNA molecule derived in part from S. pyogenes as a duplexed structure (SEQ ID NO: 46);

FIG. 1E depicts a unimolecular gRNA molecule derived in part from S. pyogenes as a duplexed structure (SEQ ID NO: 47);

FIG. 1F depicts a modular gRNA molecule derived in part from Streptococcus thermophilus (S. thermophilus) as a duplexed structure (SEQ ID NOS: 48 and 49, respectively, in order of appearance);

FIG. 1G depicts an alignment of modular gRNA molecules of S. pyogenes and S. thermophilus (SEQ ID NOS: 50-53, respectively, in order of appearance).

FIGS. 1H-1I depicts additional exemplary structures of unimolecular gRNA molecules. FIG. 1H shows an exemplary structure of a unimolecular gRNA molecule derived in part from S. pyogenes as a duplexed structure (SEQ ID NO: 45). FIG. 1I shows an exemplary structure of a unimolecular gRNA molecule derived in part from S. aureus as a duplexed structure (SEQ ID NO: 40).

FIGS. 2A-2G depict an alignment of Cas9 sequences from Chylinski et al. (RNA Biol. 2013; 10(5): 726-737). The N-terminal RuvC-like domain is boxed and indicated with a “Y”. The other two RuvC-like domains are boxed and indicated with a “B”. The HNH-like domain is boxed and indicated by a “G”. Sm: S. mutans (SEQ ID NO: 1); Sp: S. pyogenes (SEQ ID NO: 2); St: S. thermophilus (SEQ ID NO: 3); Li: L. innocua (SEQ ID NO: 4). Motif: this is a motif based on the four sequences: residues conserved in all four sequences are indicated by single letter amino acid abbreviation; “*” indicates any amino acid found in the corresponding position of any of the four sequences; and “-” indicates any amino acid, e.g., any of the 20 naturally occurring amino acids, or absent.

FIGS. 3A-3B show an alignment of the N-terminal RuvC-like domain from the Cas9 molecules disclosed in Chylinski et al (SEQ ID NOS: 54-103, respectively, in order of appearance). The last line of FIG. 3B identifies 4 highly conserved residues.

FIGS. 4A-4B show an alignment of the N-terminal RuvC-like domain from the Cas9 molecules disclosed in Chylinski et al. with sequence outliers removed (SEQ ID NOS: 104-177, respectively, in order of appearance). The last line of FIG. 4B identifies 3 highly conserved residues.

FIGS. 5A-5C show an alignment of the HNH-like domain from the Cas9 molecules disclosed in Chylinski et al (SEQ ID NOS: 178-252, respectively, in order of appearance). The last line of FIG. 5C identifies conserved residues.

FIGS. 6A-6B show an alignment of the HNH-like domain from the Cas9 molecules disclosed in Chylinski et al. with sequence outliers removed (SEQ ID NOS: 253-302, respectively, in order of appearance). The last line of FIG. 6B identifies 3 highly conserved residues.

FIGS. 7A-7B depict an alignment of Cas9 sequences from S. pyogenes and Neisseria meningitidis (N. meningitidis). The N-terminal RuvC-like domain is boxed and indicated with a “Y”. The other two RuvC-like domains are boxed and indicated with a “B”. The HNH-like domain is boxed and indicated with a “G”. Sp: S. pyogenes; Nm: N. meningitidis. Motif: this is a motif based on the two sequences: residues conserved in both sequences are indicated by a single amino acid designation; “*” indicates any amino acid found in the corresponding position of any of the two sequences; “-” indicates any amino acid, e.g., any of the 20 naturally occurring amino acids, and “-” indicates any amino acid, e.g., any of the 20 naturally occurring amino acids, or absent.

FIG. 8 shows a nucleic acid sequence encoding Cas9 of N. meningitidis (SEQ ID NO: 303). Sequence indicated by an “R” is an SV40 NLS; sequence indicated as “G” is an HA tag; and sequence indicated by an “O” is a synthetic NLS sequence; the remaining (unmarked) sequence is the open reading frame (ORF).

FIGS. 9A and 9B are schematic representations of the domain organization of S. pyogenes Cas 9. FIG. 9A shows the organization of the Cas9 domains, including amino acid positions, in reference to the two lobes of Cas9 (recognition (REC) and nuclease (NUC) lobes). FIG. 9B shows the percent homology of each domain across 83 Cas9 orthologs.

FIG. 10A is a schematic showing the plasmid map for the reporter plasmid, pAF025.

FIG. 10B is a graph showing the decrease in fluorescence from green fluorescent protein (GFP) in cells transfected with various gRNAs that target HSV-1 target sequences.

### DETAILED DESCRIPTION

### Definitions

“Domain”, as used herein, is used to describe segments of a protein or nucleic acid. Unless otherwise indicated, a domain is not required to have any specific functional property.

Calculations of homology or sequence identity between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frame shift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

“Governing gRNA molecule”, as used herein, refers to a gRNA molecule that comprises a targeting domain that is complementary to a target domain on a nucleic acid that comprises a sequence that encodes a component of the CRISPR/Cas system that is introduced into a cell or subject. A governing gRNA does not target an endogenous cell or subject sequence. In an embodiment, a governing gRNA molecule comprises a targeting domain that is complementary with a target sequence on: (a) a nucleic acid that encodes a Cas9 molecule; (b) a nucleic acid that encodes a gRNA which comprises a targeting domain that targets the UL19, UL30, UL48 or UL54 gene (a target gene gRNA); or on more than one nucleic acid that encodes a CRISPR/Cas component, e.g., both (a) and (b). In an embodiment, a nucleic acid molecule that encodes a CRISPR/Cas component, e.g., that encodes a Cas9 molecule or a target gene gRNA, comprises more than one target domain that is complementary with a governing gRNA targeting domain. While not wishing to be bound by theory, in an embodiment, it is believed that a governing gRNA molecule complexes with a Cas9 molecule and results in Cas9 mediated inactivation of the targeted nucleic acid, e.g., by cleavage or by binding to the nucleic acid, and results in cessation or reduction of the production of a CRISPR/Cas system component. In an embodiment, the Cas9 molecule forms two complexes: a complex comprising a Cas9 molecule with a target gene gRNA, which complex will alter the UL19, UL30, UL48 or UL54 gene; and a complex comprising a Cas9 molecule with a governing gRNA molecule, which complex will act to prevent further production of a CRISPR/Cas system component, e.g., a Cas9 molecule or a target gene gRNA molecule. In an embodiment, a governing gRNA molecule/Cas9 molecule complex binds to or promotes cleavage of a control region sequence, e.g., a promoter, operably linked to a sequence that encodes a Cas9 molecule, a sequence that encodes a transcribed region, an exon, or an intron, for the Cas9 molecule. In an embodiment, a governing gRNA molecule/Cas9 molecule complex binds to or promotes cleavage of a control region sequence, e.g., a promoter, operably linked to a gRNA molecule, or a sequence that encodes the gRNA molecule. In an embodiment, the governing gRNA, e.g., a Cas9-targeting governing gRNA molecule, or a target gene gRNA-targeting governing gRNA molecule, limits the effect of the Cas9 molecule/target gene gRNA molecule complex-mediated gene targeting. In an embodiment, a governing gRNA places temporal, level of expression, or other limits, on activity of the Cas9 molecule/target gene gRNA molecule complex. In an embodiment, a governing gRNA reduces off-target or other unwanted activity. In an embodiment, a governing gRNA molecule inhibits, e.g., entirely or substantially entirely inhibits, the production of a component of the Cas9 system and thereby limits, or governs, its activity.

“Modulator”, as used herein, refers to an entity, e.g., a drug, that can alter the activity (e.g., enzymatic activity, transcriptional activity, or translational activity), amount, distribution, or structure of a subject molecule or genetic sequence. In an embodiment, modulation comprises cleavage, e.g., breaking of a covalent or non-covalent bond, or the forming of a covalent or non-covalent bond, e.g., the attachment of a moiety, to the subject molecule. In an embodiment, a modulator alters the, three dimensional, secondary, tertiary, or quaternary structure, of a subject molecule. A modulator can increase, decrease, initiate, or eliminate a subject activity.

“Large molecule”, as used herein, refers to a molecule having a molecular weight of at least 2, 3, 5, 10, 20, 30, 40, 50, 60, 70, 80, 90, or 100 kD. Large molecules include proteins, polypeptides, nucleic acids, biologics, and carbohydrates.

“Polypeptide”, as used herein, refers to a polymer of amino acids having less than 100 amino acid residues. In an embodiment, it has less than 50, 20, or 10 amino acid residues.

“Reference molecule”, e.g., a reference Cas9 molecule or reference gRNA, as used herein, refers to a molecule to which a subject molecule, e.g., a subject Cas9 molecule of subject gRNA molecule, e.g., a modified or candidate Cas9 molecule is compared. For example, a Cas9 molecule can be characterized as having no more than 10% of the nuclease activity of a reference Cas9 molecule. Examples of reference Cas9 molecules include naturally occurring unmodified Cas9 molecules, e.g., a naturally occurring Cas9 molecule such as a Cas9 molecule of S. pyogenes, S. aureus or S. thermophilus. In an embodiment, the reference Cas9 molecule is the naturally occurring Cas9 molecule having the closest sequence identity or homology with the Cas9 molecule to which it is being compared. In an embodiment, the reference Cas9 molecule is a sequence, e.g., a naturally occurring or known sequence, which is the parental form on which a change, e.g., a mutation has been made.

“Replacement”, or “replaced”, as used herein with reference to a modification of a molecule does not require a process limitation but merely indicates that the replacement entity is present.

“Small molecule”, as used herein, refers to a compound having a molecular weight less than about 2 kD, e.g., less than about 2 kD, less than about 1.5 kD, less than about 1 kD, or less than about 0.75 kD.

“Subject”, as used herein, may mean either a human or non-human animal. The term includes, but is not limited to, mammals (e.g., humans, other primates, pigs, rodents (e.g., mice and rats or hamsters), rabbits, guinea pigs, cows, horses, cats, dogs, sheep, and goats). In an embodiment, the subject is a human. In other embodiments, the subject is poultry.

“Treat”, “treating” and “treatment”, as used herein, mean the treatment of a disease in a mammal, e.g., in a human, including (a) inhibiting the disease, i.e., arresting or preventing its development; (b) relieving the disease, i.e., causing regression of the disease state; and (c) curing the disease.

“Prevent”, “preventing” and “prevention”, as used herein, means the prevention of a disease in a mammal, e.g., in a human, including (a) avoiding or precluding the disease; (2) affecting the predisposition toward the disease, e.g., preventing at least one symptom of the disease or to delay onset of at least one symptom of the disease.

“X” as used herein in the context of an amino acid sequence, refers to any amino acid (e.g., any of the twenty natural amino acids) unless otherwise specified.

### Herpes Simplex Virus Type 1

Herpes simplex virus type 1 (HSV-1) causes intermittent sores of the mouth and mucous membranes. It is a ubiquitous and highly contagious pathogen. Most subjects develop the infection during childhood. By adulthood, up to 80% of the population in the United States is infected with HSV-1. Initial infection with HSV-1 generally causes painful blistering of the mucous membranes of the lips and mouth.

HSV-1 infection persists for the lifetime of the host. Primary and re-activation infections can cause permanent neurologic sequelae and blindness. There is a considerable need for methods to treat and prevent HSV1 infections.

During primary infection, the virus most often infects cells of the oropharynx and ano-genital region, causing painful vesicles in the affected region. Re-activation of HSV-1 infections most often occurs in the oropharynx and ano-genital region. However, re-activation infections of the eye and central nervous system are the most severe and damaging HSV manifestations, as they can lead to blindness and permanent neurologic disability, respectively.

HSV-1 is contained within an icosahedral particle. The virus enters the host via infection of epithelial cells within the skin and mucous membranes. The virus produces immediate early genes within the epithelial cells, which encode enzymes and binding proteins necessary for viral synthesis. After primary infection, the virus travels up sensory nerve axons via retrograde transport to the sensory dorsal root ganglion (DRG). Within the DRG, it establishes a latent infection. The latent infection persists for the lifetime of the host. Within the DRG cell, the virus uncoats, viral DNA is transported into the nucleus, and key viral RNAs associated with latency are transcribed (including the LAT RNAs).

Host immune defense is very important to combating HSV infection. CD4+ T-cells and CD8+ cells are responsible for recognizing and clearing the pathogen. Subjects with impaired T-cell responses, including those with HIV, those receiving immunosuppressants following organ transplants, and neonates with developing immune systems, are subject to the most severe manifestations of HSV-1 infections.

During the primary infection, subjects generally experience painful blistering in the oral or ano-genital region that lasts 4-15 days. The sores most commonly involve the lips, gums and nasal mucous membranes. HSV-1 primary infections may also involve the ano-genital region, including the vagina, labia, cervix, penis, scrotum, anus and skin around the thighs. Less commonly, HSV-1 primary infection may involve the eyes, central nervous system, the fingers and fingernail beds (herpetic whitlow). The infection is transmitted primarily through saliva and/or sexual activity. The blisters may break, releasing clear fluid that is highly infectious. Primary infection is often accompanied by a flu-like illness, including fever, chills and muscle aches.

Reactivations of latent infections are generally less severe and may be of shorter duration. Reactivation can affect the oral region, the ano-genital region, the eye, the central nervous system (CNS), the fingernails, and the pharynx. Reactivation generally affects the oral region but can also affect other mucous membranes, including those of the ano-genital area, fingernails, and the pharynx. Ophthalmologic disease may also occur, including epithelial keratitis, stromal keratitis and disciform keratitis. Generally, ophthalmologic manifestations of HSV-1 are self-limiting. However, HSV-1 keratitis may, in rare instances, cause scarring, secondary infection with bacterial pathogens and rarely, blindness.

In some cases, reactivation can occur in the central nervous system (CNS) via retrograde transport of the virus into the CNS. Generally, patients who are immune compromised develop HSV-1 induced encephalitis and/or meningitis. HSV-1 encephalitis or meningitis are both extremely severe. Subjects generally experience permanent neurologic damage in spite of treatment with antiviral therapy.

Primary infections and reactivation infections in the CNS, called encephalitis and meningitis, are particularly damaging. In a study of infants with HSV-encephalitis or meningitis treated with high dose antiviral therapy, there was found to be a 4% mortality rate and 69% of survivors had permanent neurologic sequelae (Kimberlin et al., Pediatrics. 2001; 108: 230-238). The majority of infants and adults who develop HSV-1 encephalitis or meningitis will experience permanent neurologic damage in spite of treatment with antiviral therapy. Reactivation infections in the CNS occur via anterograde transport of the virus into the CNS. Most commonly, patients who are immune compromised or infants develop HSV-1 induced encephalitis and/or meningitis. Healthy adults may more rarely develop HSV-1 disease of the CNS.

Reactivation infections occur in the eye via anterograde transport of the virus into the eye from the trigeminal ganglion, along the ophthalmic branch of the trigeminal nerve (the fifth cranial nerve) and into the eye. Re-activation of the virus may also occur from within the cornea. Latency within the trigeminal ganglion is established via one of two mechanisms. First, HSV-1 can travel via retrograde transport along the trigeminal nerve from the eye (after an eye infection) into the trigeminal ganglion. Alternatively, it can spread to the trigeminal ganglion via hematogenous spread following infection of the oral mucosa, genital region, or other extraocular site. After establishing latent infection of the trigeminal ganglion, at any time, particularly in the event of an immunocompromised host, the virus can re-establish infection by traveling anterograde along the trigeminal nerve and into the eye.

Ocular herpes can affect the anterior chamber of the eye, where it causes keratitis, or the posterior chamber, where it causes retinitis. In adults, HSV-1 is responsible for the majority of cases of HSV-retinitis (Pepose et al., Ocular Infection and Immunity 1996; Mosby 1155-1168). HSV-1 retinitis can lead to acute retinal necrosis (ARN), which will destroy the retina within 2 weeks without treatment (Banerjee and Rouse, Human Herpesviruses 2007; Cambridge University Press, Chapter 35). Even with treatment, the risk of permanent visual damage following ARN is higher than 50% (Roy et al., Ocular Immunology and Inflammation 2014; 22(3):170-174).

Keratitis is the most common form of ocular herpes. HSV keratitis can manifest as dentritic keratitis, stromal keratitis, blepharatis and conjunctivitis. HSV-1 is responsible for the majority of HSV-associated keratitis, accounting for 58% of cases (Dawson et. al., Suvey of Ophthalmology 1976; 21(2): 121-135). In the United States, there are approximately 48,000 cases of recurrent or primary HSV-related keratitis infections annually (Liesegang et al. 1989; 107(8): 1155-1159). Of all cases of HSV-related keratitis, approximately 1.5-3% of subjects experience severe, permanent visual impairment (Wilhelmus et. al_, Archives of Ophthalmology 1981; 99(9): 1578-82).

Overall, stromal keratitis represents approximately 15% of keratitis cases and is associated with the highest risk of permanent visual damage. Stromal keratitis results in scarring and irregular astigmatism. Previous ocular HSV infection increases the risk for developing stromal infection, which means that subjects who have had a prior ocular HSV infection have an increased risk for permanent visual damage on reactivation. In children, stromal keratitis represents up to 60% of all keratitis cases so children are particularly at risk for permanent visual damage from HSV-associated keratitis. A retrospective study in the United States from 1950-1982 found that there are approximately 2.6 new or recurrent stromal keratitis cases per 100,000 person years, or approximately 8,000 cases of stromal keratitis annually (Liesegang et. al., 1989; 107(8): 1155-1159). A more recent study in France in 2002 estimated the incidence of new or recurrent stromal keratitis cases to be 9.6 per 100,000 (Labetoulle et al., Ophthalmology 2005; 112(5):888-895). The incidence of HSV-associated keratitis may be increasing in the developed world (Farooq and Shukla 2012; Survey of Ophthalmology 57(5): 448-462).

The compositions and methods described herein can be used for the treatment and prevention of HSV-1 ocular infections, including but not limited to HSV-1 stromal keratitis, HSV-1 retinitis, HSV-1 encephalitis and HSV-1 meningitis.

Newborns are a population at particular risk for developing severe HSV-1 infections. The disease is transmitted from the mother to the fetus during childbirth. The chance of maternal-fetal transmission is highest in cases where the mother developed primary HSV infection during pregnancy. The incidence of neonatal herpes is approximately 4-30 per 100,000 births. Neonates may develop severe HSV-1 encephalitis and/or meningitis. In spite of prompt treatment with antiviral therapy, the rate of permanent neurologic sequelae in newborns infected with HSV-1 is significant.

Primary HSV-1 infections may be treated with antiviral therapy, including acyclovir, valacyclovir and famciclovir. These therapies have been demonstrated to reduce viral shedding, decrease pain and improve healing time of lesions. Re-activation of latent infections may resolve without treatment (it may be self-limiting) or may be treated with anti-viral therapy. Therapy is primarily given during acute infection. There are no curative or preventative treatments. Therapy may be given prophylactically in certain situations, including during childbirth in a mother with a recent HSV-1 infection or reactivation.

### Methods to Treat, Prevent or Reduce HSV-1 Infection

Disclosed herein are the approaches to treat, prevent, or reduce HSV-1 infection, using the compositions and methods described herein.

HSV-1 relies on the genes UL19, UL30, UL48 and/or UL54 for infection, proliferation and assembly. Knockout of any of these genes individually or in combination can prevent or treat HSV-1 infections. As the HSV-1 virus establishes latency in discrete, localized regions within the body, local delivery that delivers a treatment in the region of latency can be used. Targeting knockout to a discrete region or regions (e.g., the trigeminal dorsal root ganglion, e.g., the cervical dorsal root gangliq, e.g., the sacral dorsal root ganglia) can reduce or eliminate latent infection by disabling the HSV-1 virus.

Described herein are the approaches to treat or prevent HSV-1 by knocking out viral genes. Methods described herein include the knockout of any of the following HSV-1 encoded genes: UL19, UL30, UL48 or UL54, or any combination thereof (e.g., any two, three or all of the UL19, UL30, UL48 or UL54 gene).

UL19 (also known as VP5) encodes the HSV-1 major capsid protein, VP5. Proper assembly of the viral capsid is known to be an essential part of viral replication, assembly, maturation and infection (Homa et al., Reviews of Medical Virology 1997; 7(2):107-122). RNAi-mediated knockdown of VP5 along with another capsid capsid protein, VP23, in vitro, greatly diminished HSV-1 proliferation (Jin et al., PLoS One 2014; 9(5): e96623). Knockout of UL19 can disable HSV-1 proliferation and therefore prevent, treat or cure HSV-1 infection.

UL30 encodes the DNA polymerase catalytic subunit (HSV-1 pol). The 5′ domain of HSV-1 pol is required for viral replication. Knock out of UL30 can disable HSV-1 replication and therefore prevent and/or cure HSV-1 infection.

UL48 encodes the viral protein known as VP16 in HSV-1. VP-16 has been shown to be important in viral egress, the process by which the assembled viral capsid leaves the host nucleus and enters the cytoplasm (Mossman et al., Journal of Virology 2000; 74(14): 6287-6289). Mutation of UL48 in cell culture decreased the ability of HSV-1 to assemble efficiently (Svobodova et al., Journal of Virology 2012; 86(1): 473-483). Knockout of UL48 can disable HSV-1 assembly and egress and therefore prevent and/or cure HSV-1 infection.

UL54 encodes ICP27, a highly conserved, multi-functional protein. ICP27 is involved in transcription, RNA processing, RNA export and translation (Sandri-Goldin, Frontiers in Bioscience 2008; 13:5241-5256). ICP27 also shuts off host gene expression during HSV-1 infection. Knockout of UL54 can disable HSV-1 transcription, translation and RNA processing and therefore prevent and/or cure HSV-1 infection.

Knockout of the genes UL19, UL30, UL48 or UL54, individually or in combination, can reduce HSV-1 infectivity, replication, packaging and can therefore prevent or treat HSV-1 infection.

In addition, knock out of vital HSV-1 genes, e.g., UL19, UL30, UL48 and UL54, individually or in combination, can make HSV-1 more susceptible to antiviral therapy. Mutations in important genes can render HSV-1 and other viruses more susceptible to treatment with antivirals (Zhou et al., Journal of Virology 2014; 88(19): 11121-11129). Knocking out of UL19, UL30, UL48 and UL54, individually or in combination, may be combined with antiviral therapy to prevent or treat HSV-1 infection. The compositions and methods described herein can be used in combination with another antiviral therapy, e.g., another anti-HSV-1 therapy described herein, to treat or prevent HSV-1 infection.

In one approach, one or more of the UL19, UL30, UL48 and/or UL54 gene(s) is targeted as a targeted knockout, e.g., to inhibit essential viral functions, including, e.g. viral gene transcription, viral genome replication and viral capsid formation. In an embodiment, said approach comprises knocking out one HSV-1 gene (e.g., UL19, UL30, UL48 or UL54 gene). In another embodiment, said approach comprises knocking out two HSV-1 genes, e.g., two of UL19, UL30, UL48 or UL54 gene(s). In another embodiment, said approach comprises knocking out three HSV-1 genes, e.g., three or more of UL19, UL30, UL48 or UL54 gene(s). In another embodiment, said approach comprises knocking out four HSV-1 genes, e.g., each of UL19, UL30, UL48 and UL54 genes.

While not wishing to be bound by theory, it is considered that inhibiting essential viral functions, e.g., viral gene transcription, viral genome replication and viral capsid formation, decreases the duration of primary or recurrent infection and/or decrease shedding of viral particles. Subjects also experience shorter duration(s) of illness, decreased risk of transmission to sexual partners, decreased risk of transmission to the fetus in the case of pregnancy and/or the potential for full clearance of HSV-1 (cure).

Knockout of one or more copies (e.g., 1, 2, 3, 4, 5, 6, 7, 8, 9 or 10 copies) of one or more target genes (e.g., UL19, UL30, UL48 or UL54 gene) may be performed prior to disease onset or after disease onset, but preferably early in the disease course.

In an embodiment, the method comprises initiating treatment of a subject prior to disease onset.

In an embodiment, the method comprises initiating treatment of a subject after disease onset.

In an embodiment, the method comprises initiating treatment of a subject well after disease onset, e.g., 1, 2, 3, 4, 5, 6, 7, 8, 9, 10, 12, 16, 24, 36, 48 or more months after onset of HSV-1 infection. In an embodiment, the method comprises initiating treatment of a subject well after disease onset, e.g., 1, 2, 3, 4, 5, 10, 15, 20, 25, 40, 50 or 60 years after onset of HSV-1 infection. While not wishing to be bound by theory it is believed that this may be effective as disease progression is slow in some cases and a subject may present well into the course of illness.

In an embodiment, the method comprises initiating treatment of a subject in an advanced stage of disease, e.g., during acute or latent periods. In an embodiment, the method comprises initiating treatment of a subject in the case of severe, acute disease affecting the central nervous system, eyes, oropharynx, genital region, and/or other region.

Overall, initiation of treatment for subjects at all stages of disease is expected to improve healing, decrease duration of disease and be of benefit to subjects.

In an embodiment, the method comprises initiating treatment of a subject prior to disease expression. In an embodiment, the method comprises initiating treatment of a subject in an early stage of disease, e.g., when a subject has been exposed to HSV-1 or is thought to have been exposed to HSV-1.

In an embodiment, the method comprises initiating treatment of a subject prior to disease expression. In an embodiment, the method comprises initiating treatment of a subject in an early stage of disease, e.g., when a subject has tested positive for HSV-1 infections but has no signs or symptoms.

In an embodiment, the method comprises initiating treatment of a subject at the appearance of painful blistering in or around the mouth, e.g., oral or oropharynx, e.g., in an infant, child, adult or young adult.

In an embodiment, the method comprises initiating treatment of a subject at the appearance of painful blistering in the genital region, e.g., in an infant, child, adult or young adult.

In an embodiment, the method comprises initiating treatment of a subject suspected of having HSV-1 meningitis and/or HSV-1 encephalitis.

In an embodiment, the method comprises initiating treatment at the appearance of any of the following symptoms consistent or associated with HSV-1 meningitis and/or encephalitis: fever, headache, vomiting, photophobia, seizure, decline in level of consciousness, lethargy, or drowsiness.

In an embodiment, the method comprises initiating treatment at the appearance of any of the following signs consistent or associated with HSV meningitis and/or encephalitis: positive CSF culture for HSV-1, elevated WBC in CSF, neck stiffness/positive Brudzinski's sign. In an embodiment, the method comprises initiating treatment in a patient with signs consistent with HSV-1 encephalitis and/or meningitis on EEG, CSF exam, MRI, PCR of CSF specimen, and/or PCR of brain biopsy specimen.

In an embodiment, the method comprises initiating treatment at the appearance of any of the following symptoms consistent or associated with optic HSV-1: pain, photophobia, blurred vision, tearing, redness/injection, loss of vision, floaters, or flashes.

In an embodiment, the method comprises initiating treatment at the appearance of any of the following findings on ophthalmologic exam consistent or associated with optic HSV-1, also known as HSV-1 keratitis: small, raised clear vesicles on corneal epithelium; irregular corneal surface, punctate epithelial erosions; dense stromal infiltrate; ulceration; necrosis; focal, multifocal, or diffuse cellular infiltrates; immune rings; neovascularization; or ghost vessels at any level of the cornea.

In an embodiment, the method comprises initiating treatment at the appearance of any of the following findings on ophthalmologic exam consistent or associated with HSV-1 retinitis or acute retinal necrosis: reduced visual acuity; uveitis; vitritis; scleral injection; inflammation of the anterior and/or vitreous chamber/s; vitreous haze; optic nerve edema; peripheral retinal whitening; retinal tear; retinal detachment; retinal necrosis; evidence of occlusive vasculopathy with arterial involvement, including arterioloar sheathing and arteriolar attenuation.

In an embodiment, the method comprises initiating treatment at the appearance of symptoms and/or signs consistent or associated with either an HSV-1 or an HSV-2 infection of the eye, oropharynx, ano-genital region or central nervous system. While not wishing to be bound by theory, initiating treatment for HSV-1 infection in a case of suspected HSV-1 or HSV-2 infection early in the disease course is beneficial.

In an embodiment, the method comprises initiating treatment in utero in case of high risk of maternal-to-fetal transmission.

In an embodiment, the method comprises initiating treatment during pregnancy in case of mother who has active HSV-1 infection or has recent primary HSV-1 infection.

In an embodiment, the method comprises initiating treatment prior to organ transplantation or immediately following organ transplantation.

In an embodiment, the method comprises initiating treatment in case of suspected exposure to HSV-1.

In an embodiment, the method comprises initiating treatment prophylactically, especially in case of suspected HSV-encephalitis or meningitis.

In an embodiment, the method comprises initiating treatment of a subject who suffers from or is at risk of developing severe manifestations of HSV-1 infections, e.g., neonates, subjects with HIV, subjects who are on immunosuppressant therapy following organ transplantation, subjects who have cancer, subjects who are undergoing chemotherapy, subjects who will undergo chemotherapy, subjects who are undergoing radiation therapy, subjects who will undergo radiation therapy.

While not wishing to be bound by theory, it is considered that both HIV positive subjects and post-transplant subjects may experience severe HSV-1 activation or reactivation, including HSV-encephalitis and meningitis, due to immunodeficiency. Neonates are also at risk for severe HSV-encephalitis due to maternal-fetal transmission during childbirth. Inhibiting essential viral functions, e.g., viral gene transcription, viral genome replication and viral capsid formation, may provide superior protection to said populations at risk for severe HSV-1 infections. Subjects may experience lower rates of HSV-1 encephalitis and/or lower rates of severe neurologic sequelae following HSV-1 encephalitis, which will profoundly improve quality of life.

In an embodiment, the method comprises initiating treatment of a subject who has tested positive for HSV-1.

In an embodiment, the method comprises initiating treatment at the appearance of any one or more of the following findings consistent or associated with HSV-1: appearance of blistering in the oropharynx, ano-genital area, oral or ano-genital ulcers and/or flu-like illness.

In an embodiment, the method comprises initiating treatment at the appearance of any of the following findings consistent or associated with HSV-1 infection: fever, headache, body aches, oral or ano-genital blistering, oral ulceration, encephalitis, meningitis or keratitis.

In an embodiment, the method comprises initiating treatment in a subject who has tested positive for HSV-1 infection via viral culture, direct fluorescent antibody study, skin biopsy, PCR, blood serologic test, CSF serologic test, CSF PCR, or brain biopsy. In an embodiment, the method comprises initiating treatment in a subject who has tested positive for HSV-2 infection via diagnostic vitrectomy, endoretinal biopsy, PCR of aqueous fluid, PCR of vitreous sample.

In an embodiment, the method comprises initiating treatment in any subject exposed to HSV-1 and at high risk for severe sequelae from HSV infection.

In some embodiments, a cell is manipulated by editing (e.g., introducing a mutation in) one or more target genes, e.g., UL19, UL30, UL48 or UL54 gene. In some embodiments, the expression of one or more target genes (e.g., one or more UL19, UL30, UL48 or UL54 gene described herein) is modulated, e.g., in vivo.

In an embodiment, the method comprises delivery of gRNA by an AAV. In an embodiment, the method comprises delivery of gRNA by a lentivirus. In an embodiment, the method comprises delivery of gRNA by a nanoparticle. In an embodiment, the method comprises delivery of gRNA by a gel-based AAV for topical therapy.

In an embodiment, the method further comprising treating the subject a second antiviral therapy, e.g., an anti-HSV-1 therapy described herein. The compositions described herein can be administered concurrently with, prior to, or subsequent to, one or more additional therapies or therapeutic agents. The composition and the other therapy or therapeutic agent can be administered in any order. In an embodiment, the effect of the two treatments is synergistic. Exemplary anti-HSV-1 therapies include, but are not limited to, acyclovir, valacyclovir, famciclovir, penciclovir, or a vaccine.

Methods of Altering UL19, UL30, UL48 and/or UL54 Gene(s)

As disclosed herein, a HSV-1 target position, e.g., one or more of UL19, UL30, UL48 or UL54 gene(s), can be altered by gene editing, e.g., using CRISPR-Cas9 mediated methods as described herein.

Methods and compositions discussed herein, provide for altering a HSV-1 target position in one or more of the UL19, UL30, UL48 and/or UL54 gene(s). A HSV-1 target position can be altered by gene editing, e.g., using CRISPR-Cas9 mediated methods to alter one or more of the UL19, UL30, UL48 and/or UL54 gene(s).

An alteration of one or more of the UL19, UL30, UL48 and/or UL54 gene(s) can be mediated by any mechanism. Exemplary mechanisms that can be associated with an alteration of one or more of the UL19, UL30, UL48 and/or UL54 gene(s) include, but are not limited to, non-homologous end joining (e.g., classical or alternative), microhomology-mediated end joining (MMEJ), homology-directed repair (e.g., endogenous donor template mediated), SDSA (synthesis dependent strand annealing), single strand annealing or single strand invasion.

In an embodiment, a single strand break is introduced (e.g., positioned by one gRNA molecule) at or in close proximity to a HSV-1 target position in the UL19, UL30, UL48 and/or UL54 gene. In an embodiment, a single gRNA molecule (e.g., with a Cas9 nickase) is used to create a single strand break at or in close proximity to the HSV-1 target position, e.g., the gRNA is configured such that the single strand break is positioned either upstream (e.g., within 200 bp upstream) or downstream (e.g., within 200 bp downstream) of the HSV-1 target position. In an embodiment, the break is positioned to avoid unwanted target chromosome elements, such as repeat elements, e.g., an Alu repeat.

In an embodiment, a double strand break is introduced (e.g., positioned by one gRNA molecule) at or in close proximity to a HSV-1 target position in the UL19, UL30, UL48 and/or UL54 gene. In an embodiment, a single gRNA molecule (e.g., with a Cas9 nuclease other than a Cas9 nickase) is used to create a double strand break at or in close proximity to the HSV-1 target position, e.g., the gRNA molecule is configured such that the double strand break is positioned either upstream (e.g., within 200 bp upstream) or downstream of (e.g., within 200 bp downstream) of a HSV-1 target position. In an embodiment, the break is positioned to avoid unwanted target chromosome elements, such as repeat elements, e.g., an Alu repeat.

In an embodiment, two single strand breaks are introduced (e.g., positioned by two gRNA molecules) at or in close proximity to a HSV-1 target position in the UL19, UL30, UL48 and/or UL54 gene. In an embodiment, two gRNA molecules (e.g., with one or two Cas9 nickcases) are used to create two single strand breaks at or in close proximity to the HSV-1 target position, e.g., the gRNAs molecules are configured such that both of the single strand breaks are positioned upstream (e.g., within 200 bp upstream) or downstream (e.g., within 200 bp downstream) of the HSV-1 target position. In another embodiment, two gRNA molecules (e.g., with two Cas9 nickcases) are used to create two single strand breaks at or in close proximity to the HSV-1 target position, e.g., the gRNAs molecules are configured such that one single strand break is positioned upstream (e.g., within 200 bp upstream) and a second single strand break is positioned downstream (e.g., within 200 bp downstream) of the HSV-1 target position. In an embodiment, the breaks are positioned to avoid unwanted target chromosome elements, such as repeat elements, e.g., an Alu repeat.

In an embodiment, two double strand breaks are introduced (e.g., positioned by two gRNA molecules) at or in close proximity to a HSV-1 target position in the UL19, UL30, UL48 and/or UL54 gene. In an embodiment, two gRNA molecules (e.g., with one or two Cas9 nucleases that are not Cas9 nickases) are used to create two double strand breaks to flank a HSV-1 target position, e.g., the gRNA molecules are configured such that one double strand break is positioned upstream (e.g., within 200 bp upstream) and a second double strand break is positioned downstream (e.g., within 200 bp downstream) of the HSV-1 target position. In an embodiment, the breaks are positioned to avoid unwanted target chromosome elements, such as repeat elements, e.g., an Alu repeat.

In an embodiment, one double strand break and two single strand breaks are introduced (e.g., positioned by three gRNA molecules) at or in close proximity to a HSV-1 target position in the UL19, UL30, UL48 and/or UL54 gene. In an embodiment, three gRNA molecules (e.g., with a Cas9 nuclease other than a Cas9 nickase and one or two Cas9 nickases) to create one double strand break and two single strand breaks to flank a HSV-1 target position, e.g., the gRNA molecules are configured such that the double strand break is positioned upstream or downstream of (e.g., within 200 bp upstream or downstream) of the HSV-1 target position, and the two single strand breaks are positioned at the opposite site, e.g., downstream or upstream (within 200 bp downstream or upstream), of the HSV-1 target position. In an embodiment, the breaks are positioned to avoid unwanted target chromosome elements, such as repeat elements, e.g., an Alu repeat.

In an embodiment, four single strand breaks are introduced (e.g., positioned by four gRNA molecules) at or in close proximity to a HSV-1 target position in the UL19, UL30, UL48 and/or UL54 gene. In an embodiment, four gRNA molecule (e.g., with one or more Cas9 nickases are used to create four single strand breaks to flank a HSV-1 target position in the UL19, UL30, UL48 and/or UL54 gene, e.g., the gRNA molecules are configured such that a first and second single strand breaks are positioned upstream (e.g., within 200 bp upstream) of the HSV-1 target position, and a third and a fourth single stranded breaks are positioned downstream (e.g., within 200 bp downstream) of the HSV-1 target position. In an embodiment, the breaks are positioned to avoid unwanted target chromosome elements, such as repeat elements, e.g., an Alu repeat.

In an embodiment, two or more (e.g., three or four) gRNA molecules are used with one Cas9 molecule. In another embodiment, when two or more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

I. gRNA Molecules

A gRNA molecule, as that term is used herein, refers to a nucleic acid that promotes the specific targeting or homing of a gRNA molecule/Cas9 molecule complex to a target nucleic acid. gRNA molecules can be unimolecular (having a single RNA molecule), sometimes referred to herein as “chimeric” gRNAs, or modular (comprising more than one, and typically two, separate RNA molecules). A gRNA molecule comprises a number of domains. The gRNA molecule domains are described in more detail below.

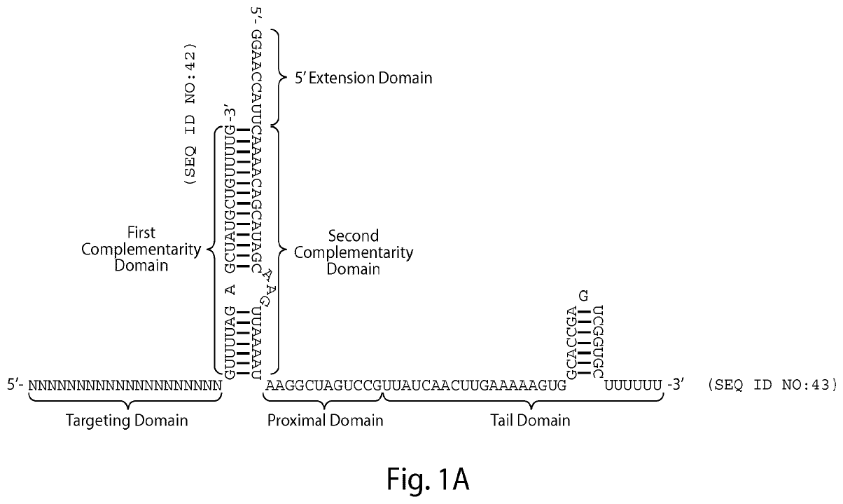

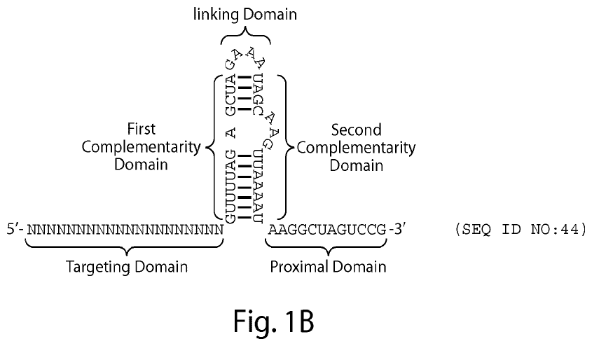

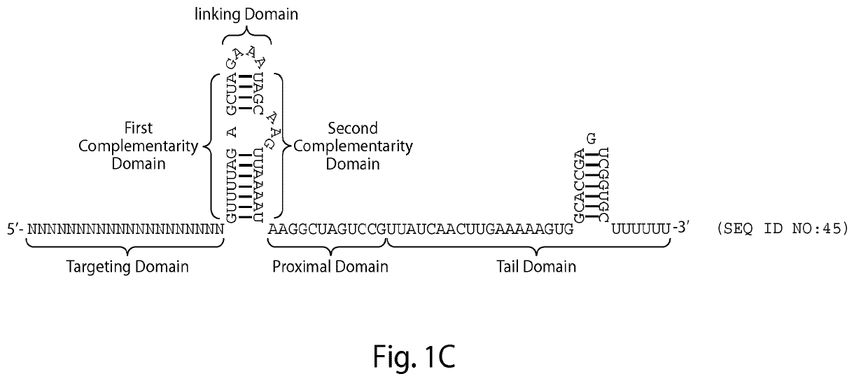

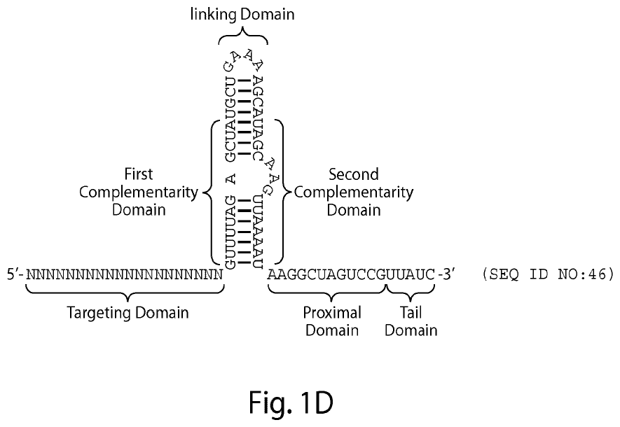

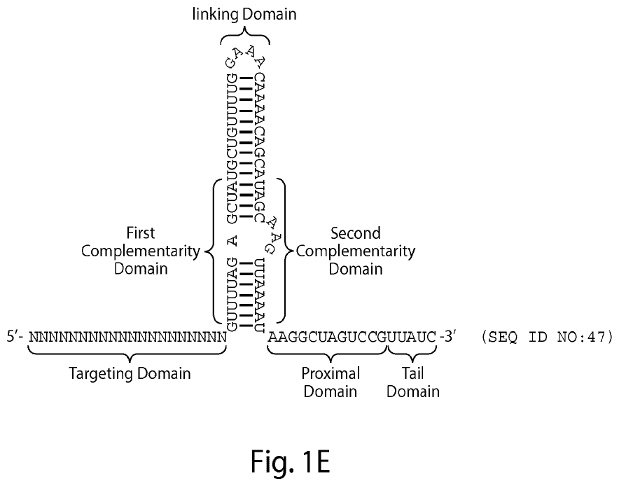

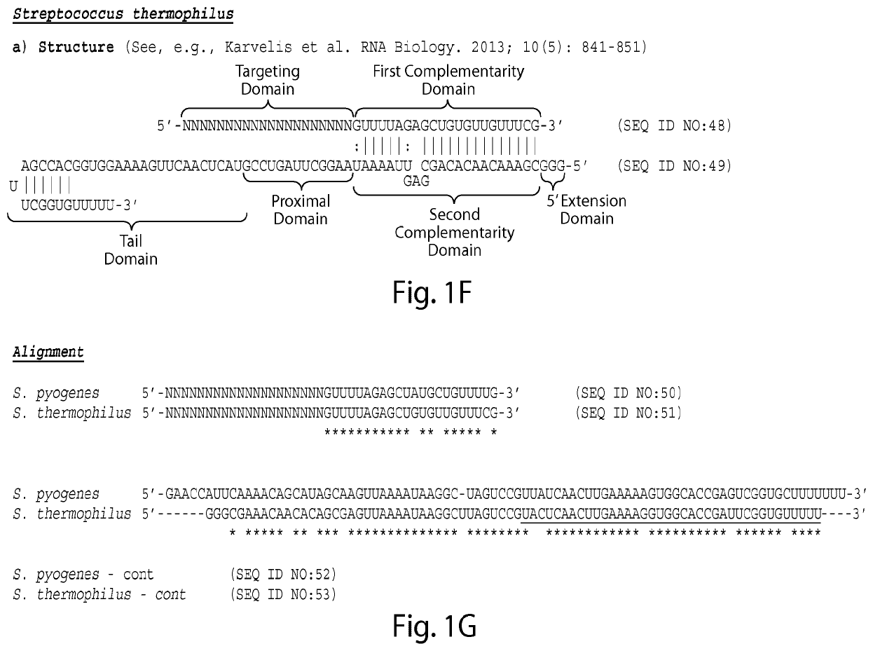

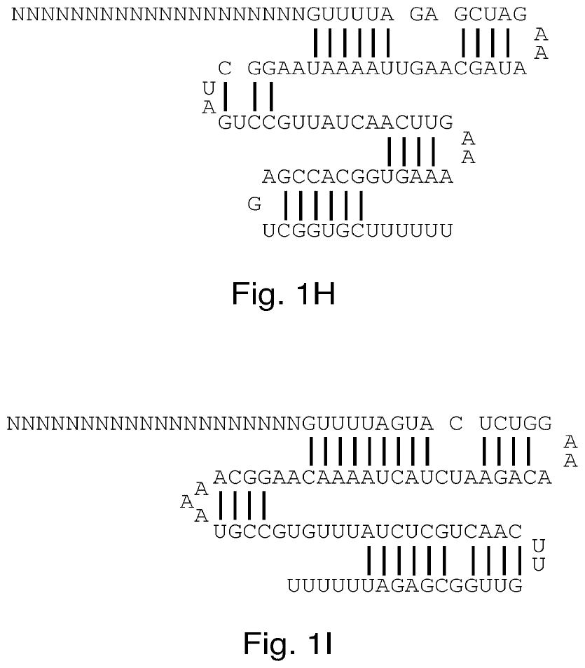

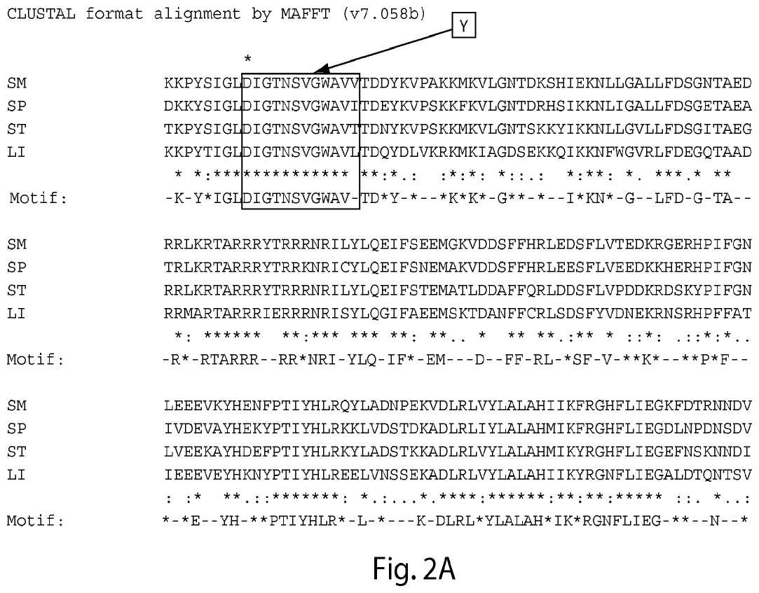

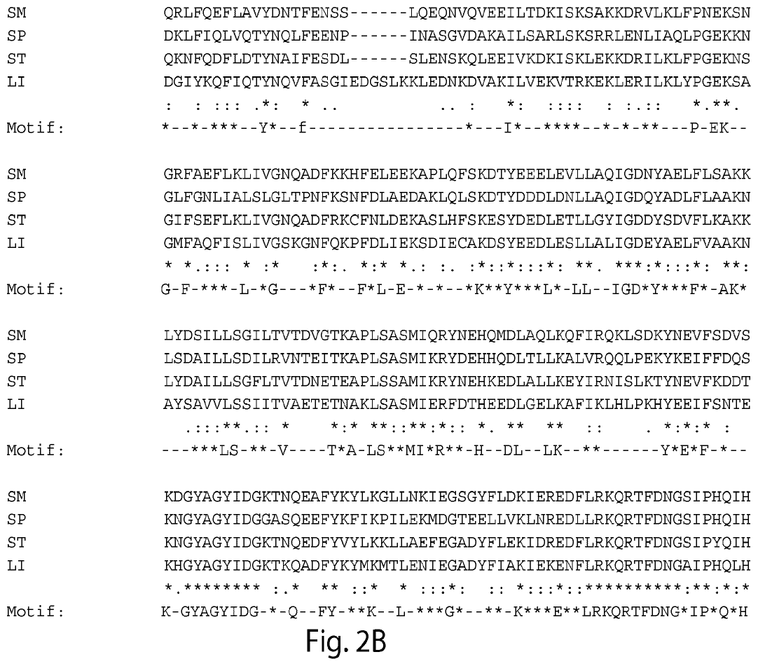

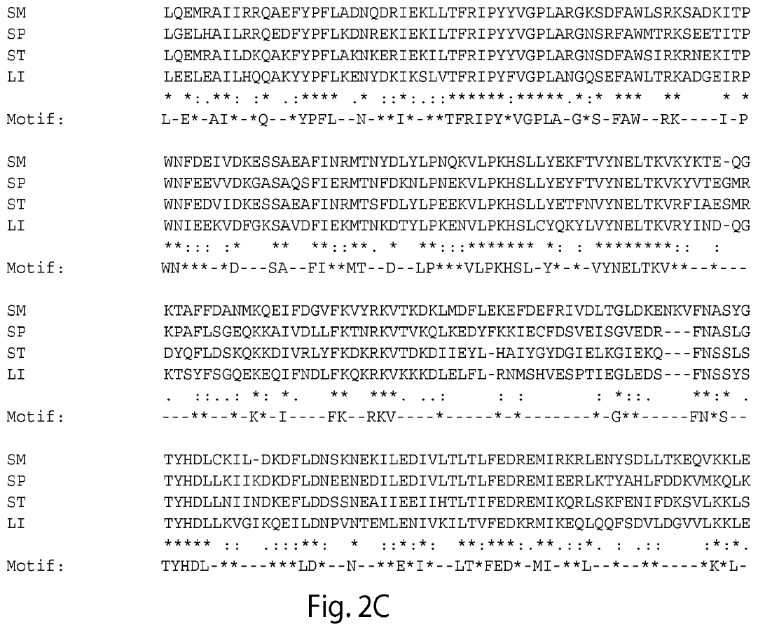

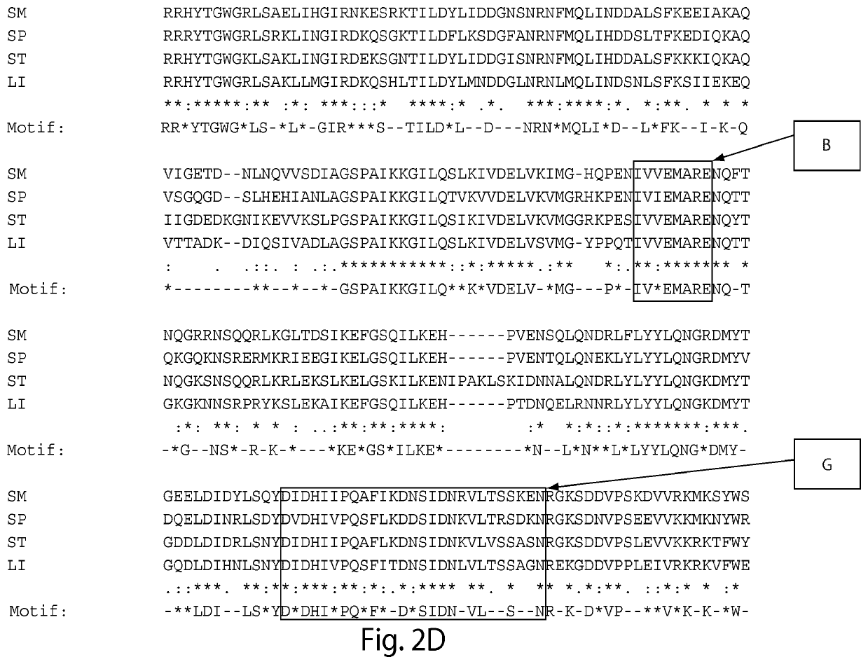

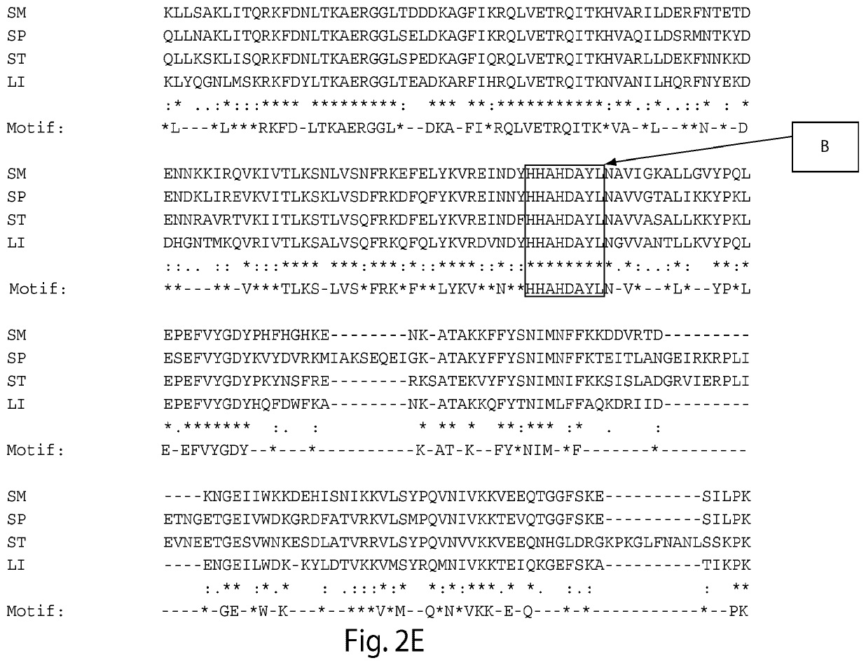

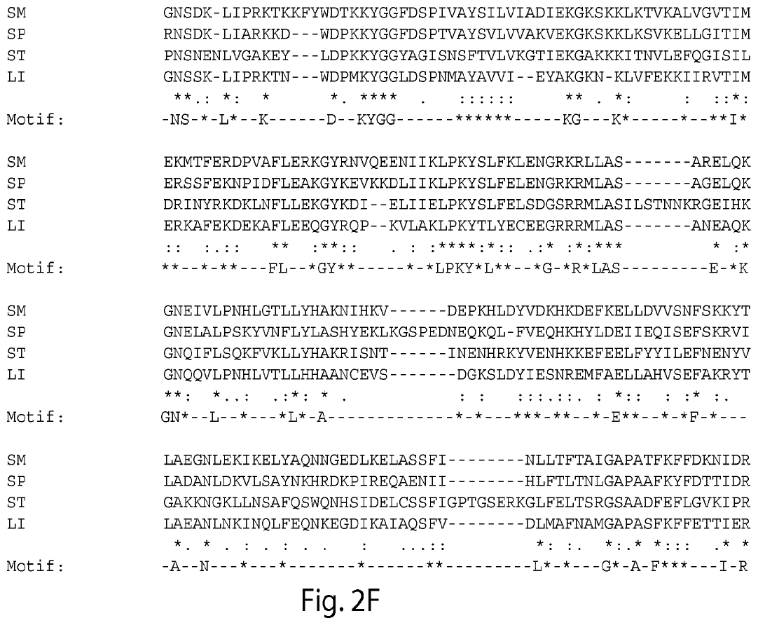

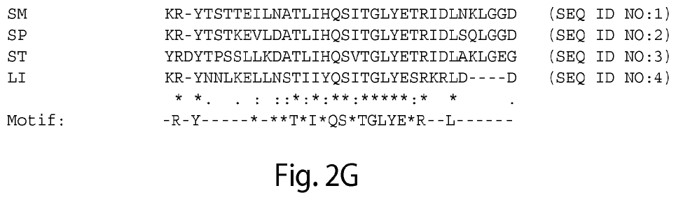

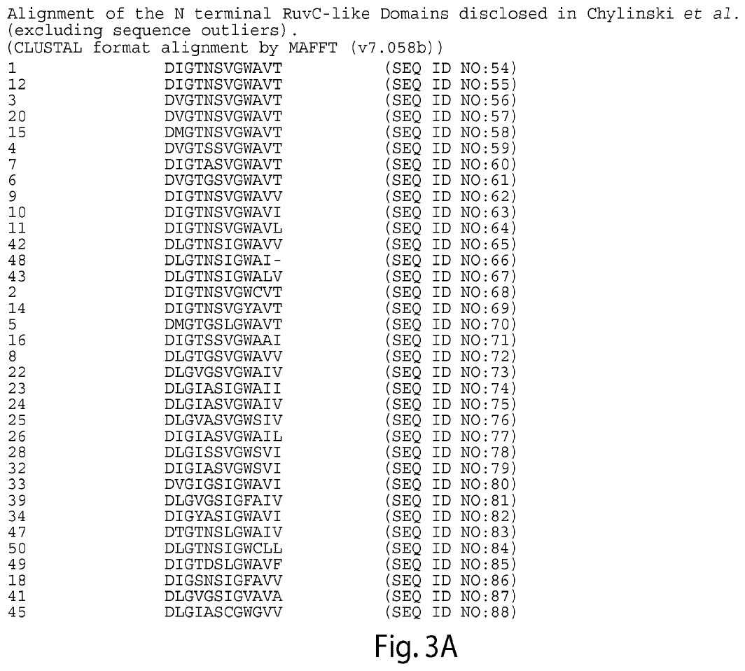

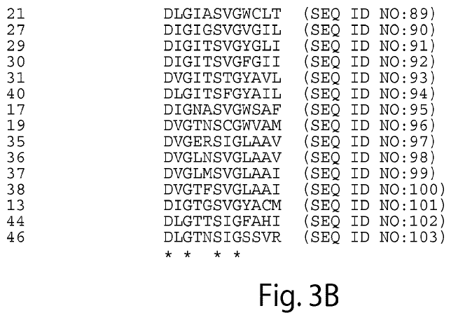

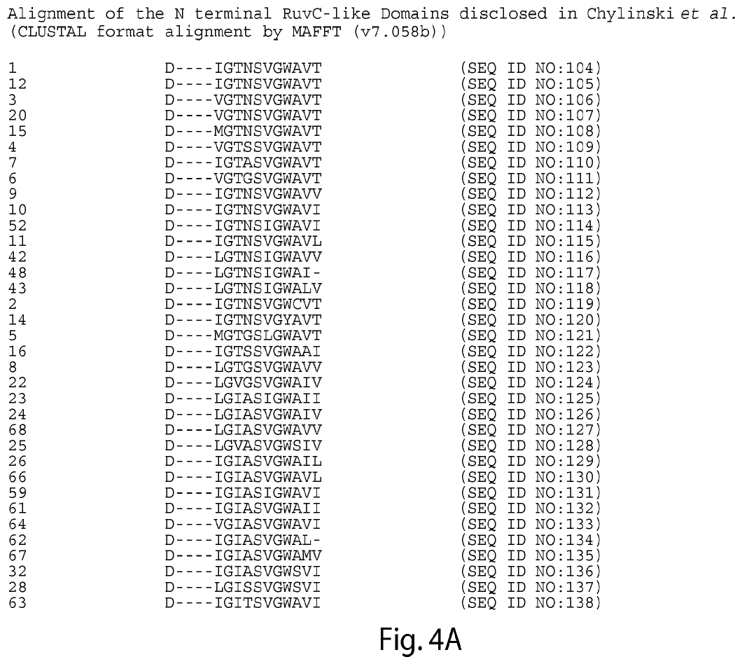

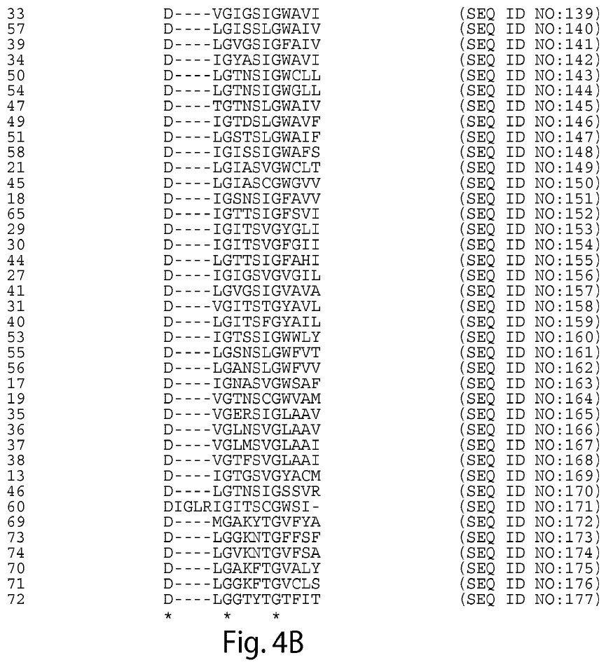

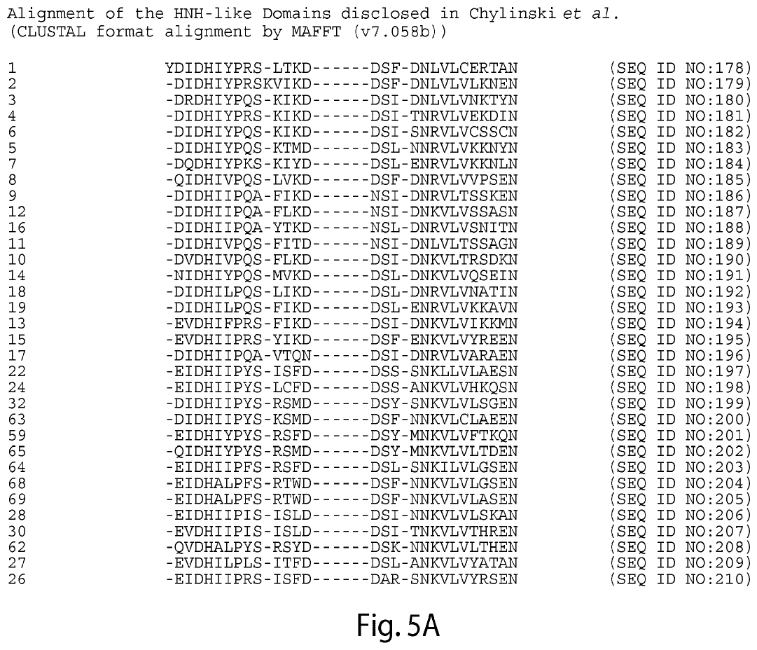

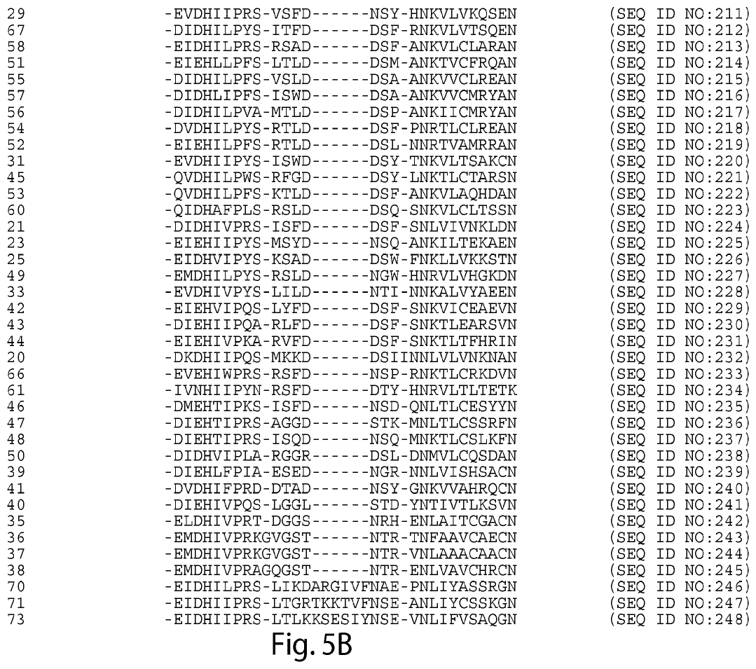

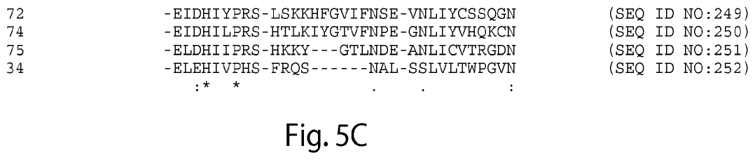

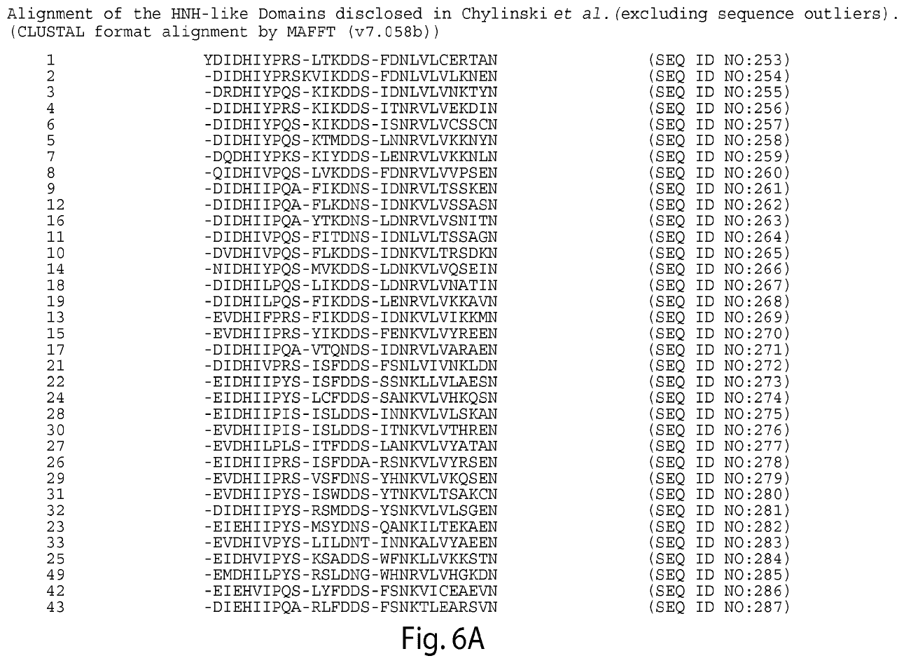

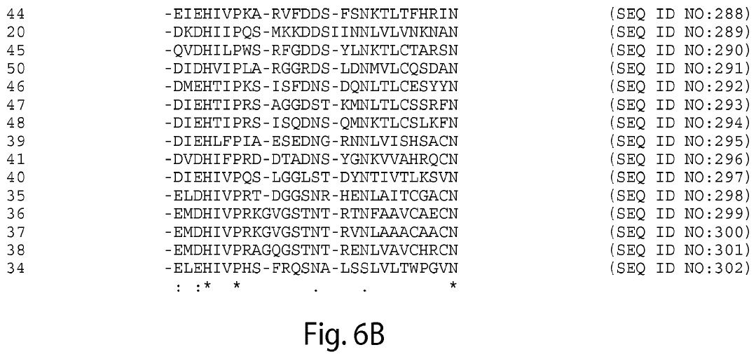

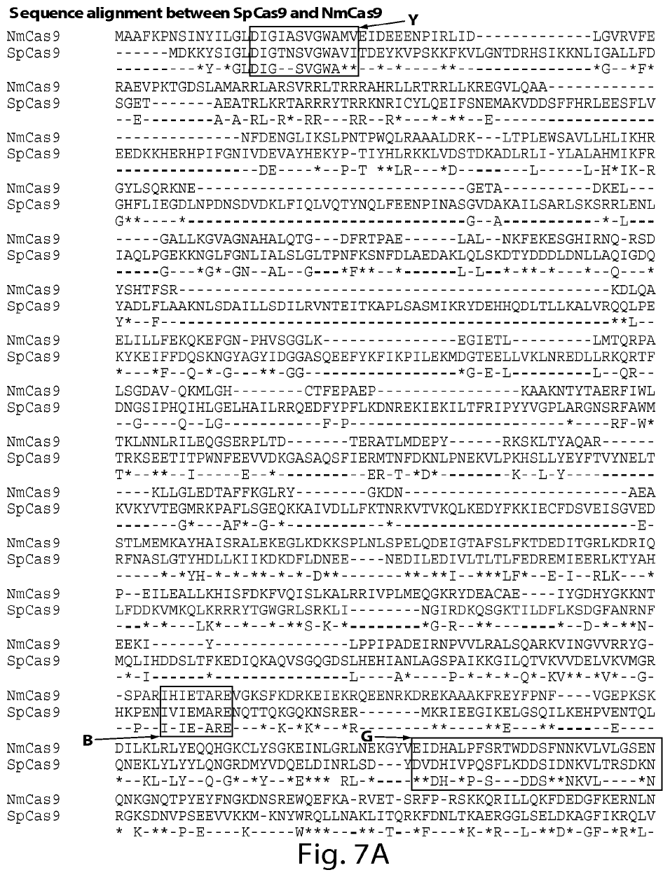

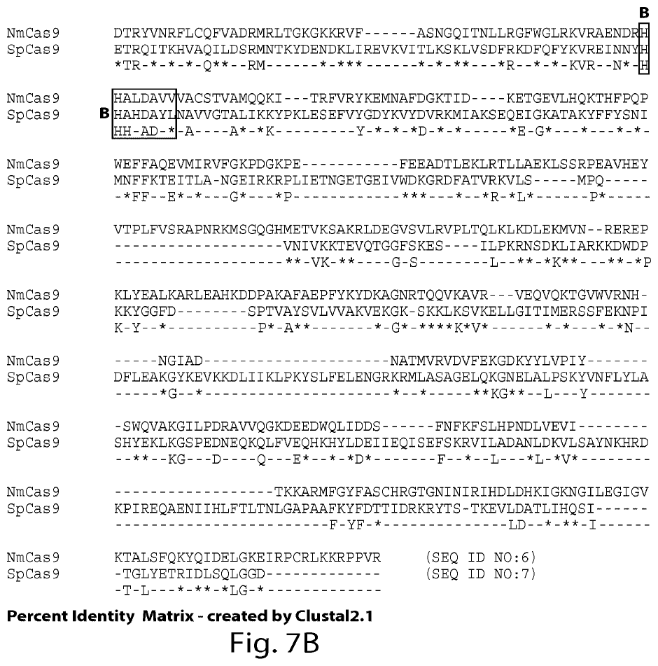

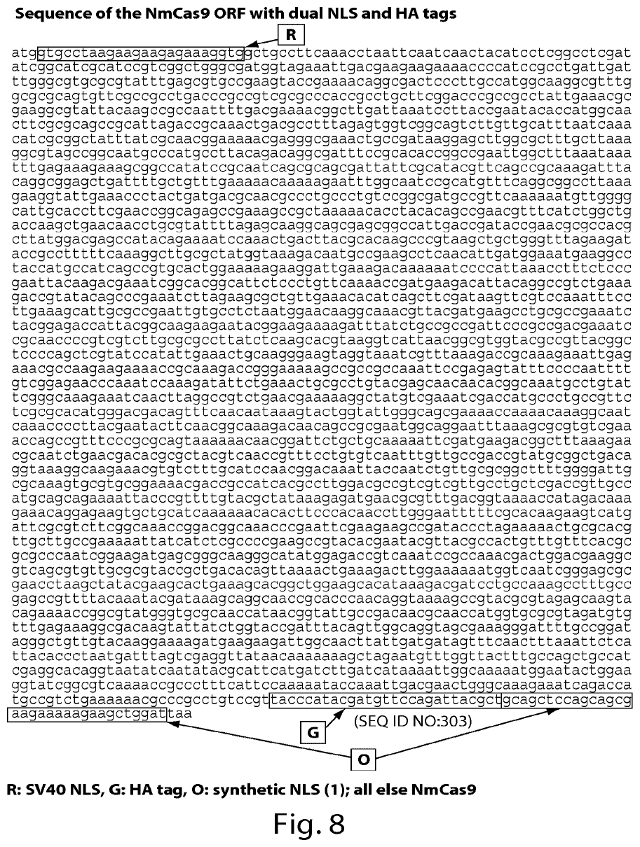

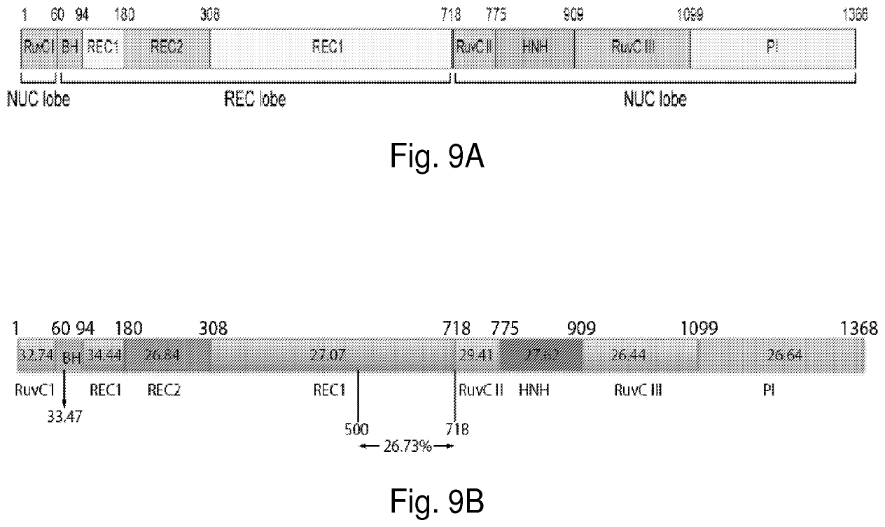

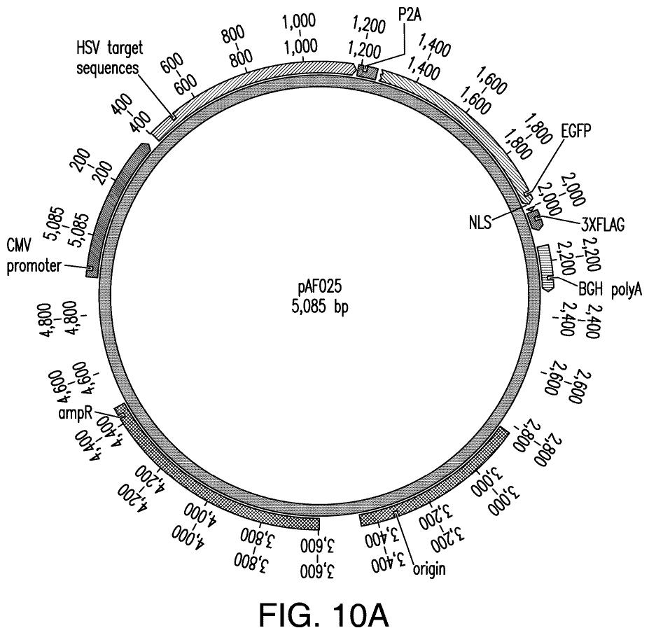

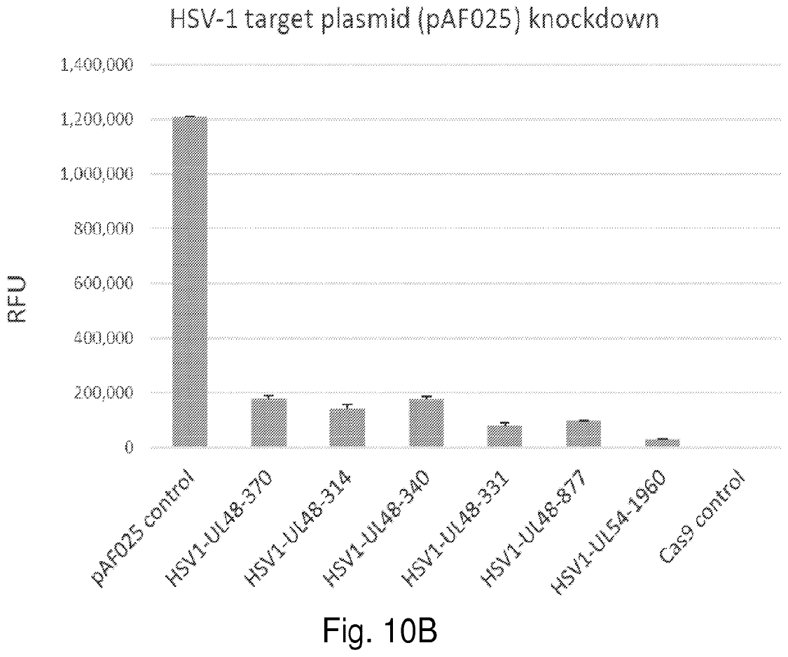

Several exemplary gRNA structures, with domains indicated thereon, are provided in FIG. 1. While not wishing to be bound by theory, in an embodiment, with regard to the three dimensional form, or intra- or inter-strand interactions of an active form of a gRNA, regions of high complementarity are sometimes shown as duplexes in FIGS. 1A-1G and other depictions provided herein.

In an embodiment, a unimolecular, or chimeric, gRNA comprises, preferably from 5′ to 3′:
a targeting domain (which is complementary to a target nucleic acid in the UL19, UL30, UL48 or UL54 gene, e.g., a targeting domain from any of Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27;
a first complementarity domain;
a linking domain;
a second complementarity domain (which is complementary to the first complementarity domain);
a proximal domain; and optionally, a tail domain.

In an embodiment, a modular gRNA comprises:
a first strand comprising, preferably from 5′ to 3′;
   a targeting domain (which is complementary to a target nucleic acid in the UL19, UL30, UL48 or UL54 gene, e.g., a targeting domain from Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C, or Table 27); and
   a first complementarity domain; and
a second strand, comprising, preferably from 5′ to 3′: optionally, a 5′ extension domain;
   a second complementarity domain;
   a proximal domain; and
   optionally, a tail domain.

The domains are discussed briefly below.

### The Targeting Domain

FIGS. 1A-1G provide examples of the placement of targeting domains.

The targeting domain comprises a nucleotide sequence that is complementary, e.g., at least 80, 85, 90, or 95% complementary, e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, in an embodiment, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence. In an embodiment, the target domain itself comprises in the 5′ to 3′ direction, an optional secondary domain, and a core domain. In an embodiment, the core domain is fully complementary with the target sequence. In an embodiment, the targeting domain is 5 to 50 nucleotides in length. The strand of the target nucleic acid with which the targeting domain is complementary is referred to herein as the complementary strand. Some or all of the nucleotides of the domain can have a modification, e.g., a modification found in Section VIII herein.

In an embodiment, the targeting domain is 16 nucleotides in length.

In an embodiment, the targeting domain is 17 nucleotides in length.

In an embodiment, the targeting domain is 18 nucleotides in length.

In an embodiment, the targeting domain is 19 nucleotides in length.

In an embodiment, the targeting domain is 20 nucleotides in length.

In an embodiment, the targeting domain is 21 nucleotides in length.

In an embodiment, the targeting domain is 22 nucleotides in length.

In an embodiment, the targeting domain is 23 nucleotides in length.

In an embodiment, the targeting domain is 24 nucleotides in length.

In an embodiment, the targeting domain is 25 nucleotides in length.

In an embodiment, the targeting domain is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

Targeting domains are discussed in more detail below.

### The First Complementarity Domain

FIGS. 1A-IG provide examples of first complementarity domains.

The first complementarity domain is complementary with the second complementarity domain, and in an embodiment, has sufficient complementarity to the second complementarity domain to form a duplexed region under at least some physiological conditions. In an embodiment, the first complementarity domain is 5 to 30 nucleotides in length. In an embodiment, the first complementarity domain is 5 to 25 nucleotides in length. In an embodiment, the first complementary domain is 7 to 25 nucleotides in length. In an embodiment, the first complementary domain is 7 to 22 nucleotides in length. In an embodiment, the first complementary domain is 7 to 18 nucleotides in length. In an embodiment, the first complementary domain is 7 to 15 nucleotides in length. In an embodiment, the first complementary domain is 5, 6, 7, 8, 9, 10, 11, 12, 13, 14, 15, 16, 17, 18, 19, 20, 21, 22, 23, 24, or 25 nucleotides in length.

In an embodiment, the first complementarity domain comprises 3 subdomains, which, in the 5′ to 3′ direction are: a 5′ subdomain, a central subdomain, and a 3′ subdomain. In an embodiment, the 5′ subdomain is 4 to 9, e.g., 4, 5, 6, 7, 8 or 9 nucleotides in length. In an embodiment, the central subdomain is 1, 2, or 3, e.g., 1, nucleotide in length. In an embodiment, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length.

The first complementarity domain can share homology with, or be derived from, a naturally occurring first complementarity domain. In an embodiment, it has at least 50% homology with a first complementarity domain disclosed herein, e.g., an S. pyogenes, S. aureus or S. thermophilus, first complementarity domain.

Some or all of the nucleotides of the domain can have a modification, e.g., a modification found in Section VIII herein.

First complementarity domains are discussed in more detail below.

### The Linking Domain

FIGS. 1A-1G provide examples of linking domains.

A linking domain serves to link the first complementarity domain with the second complementarity domain of a unimolecular gRNA. The linking domain can link the first and second complementarity domains covalently or non-covalently. In an embodiment, the linkage is covalent. In an embodiment, the linking domain covalently couples the first and second complementarity domains, see, e.g., FIGS. 1B-1E. In an embodiment, the linking domain is, or comprises, a covalent bond interposed between the first complementarity domain and the second complementarity domain. Typically the linking domain comprises one or more, e.g., 2, 3, 4, 5, 6, 7, 8, 9, or 10 nucleotides.

In modular gRNA molecules the two molecules are associated by virtue of the hybridization of the complementarity domains see e.g., FIG. 1A.

A wide variety of linking domains are suitable for use in unimolecular gRNA molecules. Linking domains can consist of a covalent bond, or be as short as one or a few nucleotides, e.g., 1, 2, 3, 4, or 5 nucleotides in length. In an embodiment, a linking domain is 2, 3, 4, 5, 6, 7, 8, 9, 10, 15, 20, or 25 or more nucleotides in length. In an embodiment, a linking domain is 2 to 50, 2 to 40, 2 to 30, 2 to 20, 2 to 10, or 2 to 5 nucleotides in length. In an embodiment, a linking domain shares homology with, or is derived from, a naturally occurring sequence, e.g., the sequence of a tracrRNA that is 5′ to the second complementarity domain. In an embodiment, the linking domain has at least 50% homology with a linking domain disclosed herein.

Some or all of the nucleotides of the domain can have a modification, e.g., a modification found in Section VIII herein.

Linking domains are discussed in more detail below.

### The 5′ Extension Domain

In an embodiment, a modular gRNA can comprise additional sequence, 5′ to the second complementarity domain, referred to herein as the 5′ extension domain, see, e.g., FIG. 1A. In an embodiment, the 5′ extension domain is, 2 to 10, 2 to 9, 2 to 8, 2 to 7, 2 to 6, 2 to 5, or 2 to 4 nucleotides in length. In an embodiment, the 5′ extension domain is 2, 3, 4, 5, 6, 7, 8, 9, or 10 or more nucleotides in length.

### The Second Complementarity Domain

FIGS. 1A-1G provide examples of second complementarity domains.

The second complementarity domain is complementary with the first complementarity domain, and in an embodiment, has sufficient complementarity to the second complementarity domain to form a duplexed region under at least some physiological conditions. In an embodiment, e.g., as shown in FIGS. 1A-1B, the second complementarity domain can include sequence that lacks complementarity with the first complementarity domain, e.g., sequence that loops out from the duplexed region.

In an embodiment, the second complementarity domain is 5 to 27 nucleotides in length. In an embodiment, it is longer than the first complementarity region. In an embodiment the second complementary domain is 7 to 27 nucleotides in length. In an embodiment, the second complementary domain is 7 to 25 nucleotides in length. In an embodiment, the second complementary domain is 7 to 20 nucleotides in length. In an embodiment, the second complementary domain is 7 to 17 nucleotides in length. In an embodiment, the complementary domain is 5, 6, 7, 8, 9, 10, 11, 12, 13, 14, 15, 16, 17, 18, 19, 20, 21, 22, 23, 24 or 25 nucleotides in length.

In an embodiment, the second complementarity domain comprises 3 subdomains, which, in the 5′ to 3′ direction are: a 5′ subdomain, a central subdomain, and a 3′ subdomain. In an embodiment, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. In an embodiment, the central subdomain is 1, 2, 3, 4 or 5, e.g., 3, nucleotides in length. In an embodiment, the 3′ subdomain is 4 to 9, e.g., 4, 5, 6, 7, 8 or 9 nucleotides in length.

In an embodiment, the 5′ subdomain and the 3′ subdomain of the first complementarity domain, are respectively, complementary, e.g., fully complementary, with the 3′ subdomain and the 5′ subdomain of the second complementarity domain.

The second complementarity domain can share homology with or be derived from a naturally occurring second complementarity domain. In an embodiment, it has at least 50% homology with a second complementarity domain disclosed herein, e.g., an S. pyogenes, S. aureus or S. thermophilus, first complementarity domain.

Some or all of the nucleotides of the domain can have a modification, e.g., a modification found in Section VIII herein.

A Proximal domain

FIGS. 1A-1G provide examples of proximal domains.

In an embodiment, the proximal domain is 5 to 20 nucleotides in length. In an embodiment, the proximal domain can share homology with or be derived from a naturally occurring proximal domain. In an embodiment, it has at least 50% homology with a proximal domain disclosed herein, e.g., an S. pyogenes, S. aureus or S. thermophilus, proximal domain.

Some or all of the nucleotides of the domain can have a modification, e.g., a modification found in Section VIII herein.

### A Tail Domain

FIGS. 1A-1G provide examples of tail domains.

As can be seen by inspection of the tail domains in FIGS. 1A-1E, a broad spectrum of tail domains are suitable for use in gRNA molecules. In an embodiment, the tail domain is 0 (absent), 1, 2, 3, 4, 5, 6, 7, 8, 9, or 10 nucleotides in length. In embodiment, the tail domain nucleotides are from or share homology with sequence from the 5′ end of a naturally occurring tail domain, see e.g., FIG. 1D or FIG. 1E. In an embodiment, the tail domain includes sequences that are complementary to each other and which, under at least some physiological conditions, form a duplexed region.

In an embodiment, the tail domain is absent or is 1 to 50 nucleotides in length. In an embodiment, the tail domain can share homology with or be derived from a naturally occurring proximal tail domain. In an embodiment, it has at least 50% homology with a tail domain disclosed herein, e.g., an S. pyogenes, S. aureus or S. thermophilus, tail domain.

In an embodiment, the tail domain includes nucleotides at the 3′ end that are related to the method of in vitro or in vivo transcription. When a T7 promoter is used for in vitro transcription of the gRNA, these nucleotides may be any nucleotides present before the 3′ end of the DNA template. When a U6 promoter is used for in vivo transcription, these nucleotides may be the sequence UUUUUU. When alternate pol-III promoters are used, these nucleotides may be various numbers or uracil bases or may include alternate bases.

The domains of gRNA molecules are described in more detail below.

### The Targeting Domain

The “targeting domain” of the gRNA is complementary to the “target domain” on the target nucleic acid. The strand of the target nucleic acid comprising the nucleotide sequence complementary to the core domain of the gRNA is referred to herein as the “complementary strand” of the target nucleic acid. Guidance on the selection of targeting domains can be found, e.g., in Fu Y et al., Nat Biotechnol 2014 (doi: 10.1038/nbt.2808) and Sternberg S H et al., Nature 2014 (doi: 10.1038/nature13011).

In an embodiment, the targeting domain is 16, 17, 18, 19, 20, 21, 22, 23, 24, 25 or 26 nucleotides in length.

In an embodiment, the targeting domain is 16 nucleotides in length.

In an embodiment, the targeting domain is 17 nucleotides in length.

In an embodiment, the targeting domain is 18 nucleotides in length.

In an embodiment, the targeting domain is 19 nucleotides in length.

In an embodiment, the targeting domain is 20 nucleotides in length.

In an embodiment, the targeting domain is 21 nucleotides in length.

In an embodiment, the targeting domain is 22 nucleotides in length.

In an embodiment, the targeting domain is 23 nucleotides in length.

In an embodiment, the targeting domain is 24 nucleotides in length.

In an embodiment, the targeting domain is 25 nucleotides in length.

In an embodiment, the targeting domain is 26 nucleotides in length.

In an embodiment, the targeting domain comprises 16 nucleotides.

In an embodiment, the targeting domain comprises 17 nucleotides.

In an embodiment, the targeting domain comprises 18 nucleotides.

In an embodiment, the targeting domain comprises 19 nucleotides.

In an embodiment, the targeting domain comprises 20 nucleotides.

In an embodiment, the targeting domain comprises 21 nucleotides.

In an embodiment, the targeting domain comprises 22 nucleotides.

In an embodiment, the targeting domain comprises 23 nucleotides.

In an embodiment, the targeting domain comprises 24 nucleotides.

In an embodiment, the targeting domain comprises 25 nucleotides.

In an embodiment, the targeting domain comprises 26 nucleotides.

In an embodiment, the targeting domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the targeting domain is 20+/−5 nucleotides in length.

In an embodiment, the targeting domain is 20+/−10, 30+/−10, 40+/−10, 50+/−10, 60+/−10, 70+/−10, 80+/−10, 90+/−10, or 100+/−10 nucleotides, in length.

In an embodiment, the targeting domain is 30+/−10 nucleotides in length.

In an embodiment, the targeting domain is 10 to 100, 10 to 90, 10 to 80, 10 to 70, 10 to 60, 10 to 50, 10 to 40, 10 to 30, 10 to 20 or 10 to 15 nucleotides in length. In another embodiment, the targeting domain is 20 to 100, 20 to 90, 20 to 80, 20 to 70, 20 to 60, 20 to 50, 20 to 40, 20 to 30, or 20 to 25 nucleotides in length.

Typically the targeting domain has full complementarity with the target sequence. In an embodiment the targeting domain has or includes 1, 2, 3, 4, 5, 6, 7 or 8 nucleotides that are not complementary with the corresponding nucleotide of the targeting domain.

In an embodiment, the target domain includes 1, 2, 3, 4 or 5 nucleotides that are complementary with the corresponding nucleotide of the targeting domain within 5 nucleotides of its 5′ end. In an embodiment, the target domain includes 1, 2, 3, 4 or 5 nucleotides that are complementary with the corresponding nucleotide of the targeting domain within 5 nucleotides of its 3′ end.

In an embodiment, the target domain includes 1, 2, 3, or 4 nucleotides that are not complementary with the corresponding nucleotide of the targeting domain within 5 nucleotides of its 5′ end. In an embodiment, the target domain includes 1, 2, 3, or 4 nucleotides that are not complementary with the corresponding nucleotide of the targeting domain within 5 nucleotides of its 3′ end.

In an embodiment, the degree of complementarity, together with other properties of the gRNA, is sufficient to allow targeting of a Cas9 molecule to the target nucleic acid.

In some embodiments, the targeting domain comprises two consecutive nucleotides that are not complementary to the target domain (“non-complementary nucleotides”), e.g., two consecutive noncomplementary nucleotides that are within 5 nucleotides of the 5′ end of the targeting domain, within 5 nucleotides of the 3′ end of the targeting domain, or more than 5 nucleotides away from one or both ends of the targeting domain.

In an embodiment, no two consecutive nucleotides within 5 nucleotides of the 5′ end of the targeting domain, within 5 nucleotides of the 3′ end of the targeting domain, or within a region that is more than 5 nucleotides away from one or both ends of the targeting domain, are not complementary to the targeting domain.

In an embodiment, there are no noncomplementary nucleotides within 5 nucleotides of the 5′ end of the targeting domain, within 5 nucleotides of the 3′ end of the targeting domain, or within a region that is more than 5 nucleotides away from one or both ends of the targeting domain.

In an embodiment, the targeting domain nucleotides do not comprise modifications, e.g., modifications of the type provided in Section VIII. However, in an embodiment, the targeting domain comprises one or more modifications, e.g., modifications that it render it less susceptible to degradation or more bio-compatible, e.g., less immunogenic. By way of example, the backbone of the targeting domain can be modified with a phosphorothioate, or other modification(s) from Section VIII. In an embodiment, a nucleotide of the targeting domain can comprise a 2′ modification, e.g., a 2-acetylation, e.g., a 2′ methylation, or other modification(s) from Section VIII.

In some embodiments, the targeting domain includes 1, 2, 3, 4, 5, 6, 7 or 8 or more modifications. In an embodiment, the targeting domain includes 1, 2, 3, or 4 modifications within 5 nucleotides of its 5′ end. In an embodiment, the targeting domain comprises as many as 1, 2, 3, or 4 modifications within 5 nucleotides of its 3′ end.

In some embodiments, the targeting domain comprises modifications at two consecutive nucleotides, e.g., two consecutive nucleotides that are within 5 nucleotides of the 5′ end of the targeting domain, within 5 nucleotides of the 3′ end of the targeting domain, or more than 5 nucleotides away from one or both ends of the targeting domain.

In an embodiment, no two consecutive nucleotides are modified within 5 nucleotides of the 5′ end of the targeting domain, within 5 nucleotides of the 3′ end of the targeting domain, or within a region that is more than 5 nucleotides away from one or both ends of the targeting domain. In an embodiment, no nucleotide is modified within 5 nucleotides of the 5′ end of the targeting domain, within 5 nucleotides of the 3′ end of the targeting domain, or within a region that is more than 5 nucleotides away from one or both ends of the targeting domain.

Modifications in the targeting domain can be selected so as to not interfere with targeting efficacy, which can be evaluated by testing a candidate modification in the system described in Section IV. gRNAs having a candidate targeting domain having a selected length, sequence, degree of complementarity, or degree of modification, can be evaluated in a system in Section IV. The candidate targeting domain can be placed, either alone, or with one or more other candidate changes in a gRNA molecule/Cas9 molecule system known to be functional with a selected target and evaluated.

In an embodiment, all of the modified nucleotides are complementary to and capable of hybridizing to corresponding nucleotides present in the target domain. In another embodiment, 1, 2, 3, 4, 5, 6, 7 or 8 or more modified nucleotides are not complementary to or capable of hybridizing to corresponding nucleotides present in the target domain.

In an embodiment, the targeting domain comprises, preferably in the 5′-3′ direction: a secondary domain and a core domain. These domains are discussed in more detail below.

### The Core Domain and Secondary Domain of the Targeting Domain

The “core domain” of the targeting domain is complementary to the “core domain target” on the target nucleic acid. In an embodiment, the core domain comprises about 8 to about 13 nucleotides from the 3′ end of the targeting domain (e.g., the most 3′ 8 to 13 nucleotides of the targeting domain).

In an embodiment, the core domain and targeting domain, are independently, 6+/−2, 7+/−2, 8+/−2, 9+/−2, 10+/−2, 11+/−2, 12+/−2, 13+/−2, 14+/−2, 15+/−2, or 16+−2, nucleotides in length.

In an embodiment, the core domain and targeting domain, are independently, 10+/−2 nucleotides in length.

In an embodiment, the core domain and targeting domain, are independently, 10+/−4 nucleotides in length.

In an embodiment, the core domain and targeting domain are independently 6, 7, 8, 9, 10, 11, 12, 13, 14, 15, 16, 17, or 18 nucleotides in length.

In an embodiment, the core domain and targeting domain are independently 3 to 20, 4 to 20, 5 to 20, 6 to 20, 7 to 20, 8 to 20, 9 to 20, 10 to 20 or 15 to 20 nucleotides in length.

In an embodiment, the core domain and targeting domain are independently 3 to 15, e.g., 6 to 15, 7 to 14, 7 to 13, 6 to 12, 7 to 12, 7 to 11, 7 to 10, 8 to 14, 8 to 13, 8 to 12, 8 to 11, 8 to 10 or 8 to 9 nucleotides in length.

The core domain is complementary with the core domain target. Typically the core domain has exact complementarity with the core domain target. In some embodiments, the core domain can have 1, 2, 3, 4 or 5 nucleotides that are not complementary with the corresponding nucleotide of the core domain. In an embodiment, the degree of complementarity, together with other properties of the gRNA, is sufficient to allow targeting of a Cas9 molecule to the target nucleic acid.

The “secondary domain” of the targeting domain of the gRNA is complementary to the “secondary domain target” of the target nucleic acid.

In an embodiment, the secondary domain is positioned 5′ to the core domain.

In an embodiment, the secondary domain is absent or optional.

In an embodiment, if the targeting domain is 26 nucleotides in length and the core domain (counted from the 3′ end of the targeting domain) is 8 to 13 nucleotides in length, the secondary domain is 12 to 17 nucleotides in length.

In an embodiment, if the targeting domain is 25 nucleotides in length and the core domain (counted from the 3′ end of the targeting domain) is 8 to 13 nucleotides in length, the secondary domain is 12 to 17 nucleotides in length.

In an embodiment, if the targeting domain is 24 nucleotides in length and the core domain (counted from the 3′ end of the targeting domain) is 8 to 13 nucleotides in length, the secondary domain is 11 to 16 nucleotides in length.

In an embodiment, if the targeting domain is 23 nucleotides in length and the core domain (counted from the 3′ end of the targeting domain) is 8 to 13 nucleotides in length, the secondary domain is 10 to 15 nucleotides in length.

In an embodiment, if the targeting domain is 22 nucleotides in length and the core domain (counted from the 3′ end of the targeting domain) is 8 to 13 nucleotides in length, the secondary domain is 9 to 14 nucleotides in length.

In an embodiment, if the targeting domain is 21 nucleotides in length and the core domain (counted from the 3′ end of the targeting domain) is 8 to 13 nucleotides in length, the secondary domain is 8 to 13 nucleotides in length.

In an embodiment, if the targeting domain is 20 nucleotides in length and the core domain (counted from the 3′ end of the targeting domain) is 8 to 13 nucleotides in length, the secondary domain is 7 to 12 nucleotides in length.

In an embodiment, if the targeting domain is 19 nucleotides in length and the core domain (counted from the 3′ end of the targeting domain) is 8 to 13 nucleotides in length, the secondary domain is 6 to 11 nucleotides in length.

In an embodiment, if the targeting domain is 18 nucleotides in length and the core domain (counted from the 3′ end of the targeting domain) is 8 to 13 nucleotides in length, the secondary domain is 5 to 10 nucleotides in length.

In an embodiment, if the targeting domain is 17 nucleotides in length and the core domain (counted from the 3′ end of the targeting domain) is 8 to 13 nucleotides in length, the secondary domain is 4 to 9 nucleotides in length.

In an embodiment, if the targeting domain is 16 nucleotides in length and the core domain (counted from the 3′ end of the targeting domain) is 8 to 13 nucleotides in length, the secondary domain is 3 to 8 nucleotides in length.

In an embodiment, the secondary domain is 0, 1, 2, 3, 4, 5, 6, 7, 8, 9, 10, 11, 12, 13, 14 or 15 nucleotides in length.

The secondary domain is complementary with the secondary domain target. Typically the secondary domain has exact complementarity with the secondary domain target. In some embodiments the secondary domain can have 1, 2, 3, 4 or 5 nucleotides that are not complementary with the corresponding nucleotide of the secondary domain. In an embodiment, the degree of complementarity, together with other properties of the gRNA, is sufficient to allow targeting of a Cas9 molecule to the target nucleic acid.

In an embodiment, the core domain nucleotides do not comprise modifications, e.g., modifications of the type provided in Section VIII. However, in an embodiment, the core domain comprises one or more modifications, e.g., modifications that it render it less susceptible to degradation or more bio-compatible, e.g., less immunogenic. By way of example, the backbone of the core domain can be modified with a phosphorothioate, or other modification(s) from Section VIII. In an embodiment a nucleotide of the core domain can comprise a 2′ modification, e.g., a 2-acetylation, e.g., a 2′ methylation, or other modification(s) from Section VIII. Typically, a core domain will contain no more than 1, 2, or 3 modifications.

Modifications in the core domain can be selected so as to not interfere with targeting efficacy, which can be evaluated by testing a candidate modification in the system described in Section IV. gRNAs having a candidate core domain having a selected length, sequence, degree of complementarity, or degree of modification, can be evaluated in the system described at Section IV. The candidate core domain can be placed, either alone, or with one or more other candidate changes in a gRNA molecule/Cas9 molecule system known to be functional with a selected target and evaluated.

In an embodiment, the secondary domain nucleotides do not comprise modifications, e.g., modifications of the type provided in Section VIII. However, in an embodiment, the secondary domain comprises one or more modifications, e.g., modifications that render it less susceptible to degradation or more bio-compatible, e.g., less immunogenic. By way of example, the backbone of the secondary domain can be modified with a phosphorothioate, or other modification(s) from Section VIII. In an embodiment a nucleotide of the secondary domain can comprise a 2′ modification, e.g., a 2-acetylation, e.g., a 2′ methylation, or other modification(s) from Section VIII. Typically, a secondary domain will contain no more than 1, 2, or 3 modifications.

Modifications in the secondary domain can be selected to not interfere with targeting efficacy, which can be evaluated by testing a candidate modification in the system described in Section IV. gRNAs having a candidate secondary domain having a selected length, sequence, degree of complementarity, or degree of modification, can be evaluated in the system described at Section IV. The candidate secondary domain can be placed, either alone, or with one or more other candidate changes in a gRNA molecule/Cas9 molecule system known to be functional with a selected target and evaluated.

In an embodiment, (1) the degree of complementarity between the core domain and its target, and (2) the degree of complementarity between the secondary domain and its target, may differ. In an embodiment, (1) may be greater than (2). In an embodiment, (1) may be less than (2). In an embodiment, (1) and (2) are the same, e.g., each may be completely complementary with its target.

In an embodiment, (1) the number of modifications (e.g., modifications from Section VIII) of the nucleotides of the core domain and (2) the number of modifications (e.g., modifications from Section VIII) of the nucleotides of the secondary domain may differ. In an embodiment, (1) may be less than (2). In an embodiment, (1) may be greater than (2). In an embodiment, (1) and (2) may be the same, e.g., each may be free of modifications.

### The First and Second Complementarity Domains

The first complementarity domain is complementary with the second complementarity domain.

Typically the first domain does not have exact complementarity with the second complementarity domain target. In some embodiments, the first complementarity domain can have 1, 2, 3, 4 or 5 nucleotides that are not complementary with the corresponding nucleotide of the second complementarity domain. In an embodiment, 1, 2, 3, 4, 5 or 6, e.g., 3 nucleotides, will not pair in the duplex, and, e.g., form a non-duplexed or looped-out region. In an embodiment, an unpaired, or loop-out, region, e.g., a loop-out of 3 nucleotides, is present on the second complementarity domain. In an embodiment, the unpaired region begins 1, 2, 3, 4, 5, or 6, e.g., 4, nucleotides from the 5′ end of the second complementarity domain.

In an embodiment, the degree of complementarity, together with other properties of the gRNA, is sufficient to allow targeting of a Cas9 molecule to the target nucleic acid.

In an embodiment, the first and second complementarity domains are:
independently, 6+/−2, 7+/−2, 8+/−2, 9+/−2, 10+/−2, 11+/−2, 12+/−2, 13+/−2, 14+/−2, 15+/−2, 16+/−2, 17+/−2, 18+/−2, 19+/−2, or 20+/−2, 21+/−2, 22+/−2, 23+/−2, or 24+/−2 nucleotides in length;
independently, 6, 7, 8, 9, 10, 11, 12, 13, 14, 14, 16, 17, 18, 19, 20, 21, 22, 23, 24, 25, or 26 nucleotides in length; or
independently, 5 to 24, 5 to 23, 5 to 22, 5 to 21, 5 to 20, 7 to 18, 9 to 16, or 10 to 14 nucleotides in length.

In an embodiment, the second complementarity domain is longer than the first complementarity domain, e.g., 2, 3, 4, 5, or 6, e.g., 6, nucleotides longer.

In an embodiment, the first and second complementary domains, independently, do not comprise modifications, e.g., modifications of the type provided in Section VIII.

In an embodiment, the first and second complementary domains, independently, comprise one or more modifications, e.g., modifications that the render the domain less susceptible to degradation or more bio-compatible, e.g., less immunogenic. By way of example, the backbone of the domain can be modified with a phosphorothioate, or other modification(s) from Section VIII. In an embodiment a nucleotide of the domain can comprise a 2′ modification, e.g., a 2-acetylation, e.g., a 2′ methylation, or other modification(s) from Section VIII.

In an embodiment, the first and second complementary domains, independently, include 1, 2, 3, 4, 5, 6, 7 or 8 or more modifications. In an embodiment, the first and second complementary domains, independently, include 1, 2, 3, or 4 modifications within 5 nucleotides of its 5′ end. In an embodiment, the first and second complementary domains, independently, include as many as 1, 2, 3, or 4 modifications within 5 nucleotides of its 3′ end.

In an embodiment, the first and second complementary domains, independently, include modifications at two consecutive nucleotides, e.g., two consecutive nucleotides that are within 5 nucleotides of the 5′ end of the domain, within 5 nucleotides of the 3′ end of the domain, or more than 5 nucleotides away from one or both ends of the domain. In an embodiment, the first and second complementary domains, independently, include no two consecutive nucleotides that are modified, within 5 nucleotides of the 5′ end of the domain, within 5 nucleotides of the 3′ end of the domain, or within a region that is more than 5 nucleotides away from one or both ends of the domain. In an embodiment, the first and second complementary domains, independently, include no nucleotide that is modified within 5 nucleotides of the 5′ end of the domain, within 5 nucleotides of the 3′ end of the domain, or within a region that is more than 5 nucleotides away from one or both ends of the domain.

Modifications in a complementarity domain can be selected so as to not interfere with targeting efficacy, which can be evaluated by testing a candidate modification in the system described in Section IV. gRNAs having a candidate complementarity domain having a selected length, sequence, degree of complementarity, or degree of modification, can be evaluated in the system described in Section IV. The candidate complementarity domain can be placed, either alone, or with one or more other candidate changes in a gRNA molecule/Cas9 molecule system known to be functional with a selected target and evaluated.

In an embodiment, the first complementarity domain has at least 60, 70, 80, 85%, 90% or 95% homology with, or differs by no more than 1, 2, 3, 4, 5, or 6 nucleotides from, a reference first complementarity domain, e.g., a naturally occurring, e.g., an S. pyogenes, S. aureus or S. thermophilus, first complementarity domain, or a first complementarity domain described herein, e.g., from FIGS. 1A-1G.

In an embodiment, the second complementarity domain has at least 60, 70, 80, 85%, 90%, or 95% homology with, or differs by no more than 1, 2, 3, 4, 5, or 6 nucleotides from, a reference second complementarity domain, e.g., a naturally occurring, e.g., an S. pyogenes, S. aureus or S. thermophilus, second complementarity domain, or a second complementarity domain described herein, e.g., from FIGS. 1A-1G.

The duplexed region formed by first and second complementarity domains is typically 6, 7, 8, 9, 10, 11, 12, 13, 14, 15, 16, 17, 18, 19, 20, 21 or 22 base pairs in length (excluding any looped out or unpaired nucleotides).

In some embodiments, the first and second complementarity domains, when duplexed, comprise 11 paired nucleotides, for example, in the gRNA sequence (one paired strand underlined, one bolded):

(SEQ ID NO: 5)
NNNNNNNNNNNNNNNNNNNNGUUUUAGAGCUAGAAAUAGCAAGUUAAAAU
AAGGCUAGUCCGUUAUCAACUUGAAAAAGUGGCACCGAGUCGGUGC.

In some embodiments, the first and second complementarity domains, when duplexed, comprise 15 paired nucleotides, for example in the gRNA sequence (one paired strand underlined, one bolded):

(SEQ ID NO: 27)
NNNNNNNNNNNNNNNNNNNNGUUUUAGAGCUAUGCUGAAAAGCAUAGCAA
GUUAAAAUAAGGCUAGUCCGUUAUCAACUUGAAAAAGUGGCACCGAGUCG
GUGC.

In some embodiments the first and second complementarity domains, when duplexed, comprise 16 paired nucleotides, for example in the gRNA sequence (one paired strand underlined, one bolded):

(SEQ ID NO: 28)
NNNNNNNNNNNNNNNNNNNNGUUUUAGAGCUAUGCUGGAAACAGCAUAGC
AAGUUAAAAUAAGGCUAGUCCGUUAUCAACUUGAAAAAGUGGCACCGAGU
CGGUGC.

In some embodiments the first and second complementarity domains, when duplexed, comprise 21 paired nucleotides, for example in the gRNA sequence (one paired strand underlined, one bolded):

(SEQ ID NO: 29)
NNNNNNNNNNNNNNNNNNNNGUUUUAGAGCUAUGCUGUUUUGGAAACAAA
ACAGCAUAGCAAGUUAAAAUAAGGCUAGUCCGUUAUCAACUUGAAAAAGU
GGCACCGAGUCGGUGC.

In some embodiments, nucleotides are exchanged to remove poly-U tracts, for example in the gRNA sequences (exchanged nucleotides underlined):

(SEQ ID NO: 30)
NNNNNNNNNNNNNNNNNNNNGUAUUAGAGCUAGAAAUAGCAAGUUAAUAU
AAGGCUAGUCCGUUAUCAACUUGAAAAAGUGGCACCGAGUCGGUGC;
(SEQ ID NO: 31)
NNNNNNNNNNNNNNNNNNNNGUUUAAGAGCUAGAAAUAGCAAGUUUAAAU
AAGGCUAGUCCGUUAUCAACUUGAAAAAGUGGCACCGAGUCGGUGC;
or
(SEQ ID NO: 32)
NNNNNNNNNNNNNNNNNNNNGUAUUAGAGCUAUGCUGUAUUGGAAACAAU
ACAGCAUAGCAAGUUAAUAUAAGGCUAGUCCGUUAUCAACUUGAAAAAGU
GGCACCGAGUCGGUGC.

### The 5′ Extension Domain

In an embodiment, a modular gRNA can comprise additional sequence, 5′ to the second complementarity domain. In an embodiment, the 5′ extension domain is 2 to 10, 2 to 9, 2 to 8, 2 to 7, 2 to 6, 2 to 5, or 2 to 4 nucleotides in length. In an embodiment, the 5′ extension domain is 2, 3, 4, 5, 6, 7, 8, 9, or 10 or more nucleotides in length.

In an embodiment, the 5′ extension domain nucleotides do not comprise modifications, e.g., modifications of the type provided in Section VIII. However, in an embodiment, the 5′ extension domain comprises one or more modifications, e.g., modifications that it render it less susceptible to degradation or more bio-compatible, e.g., less immunogenic. By way of example, the backbone of the 5′ extension domain can be modified with a phosphorothioate, or other modification(s) from Section VIII. In an embodiment, a nucleotide of the 5′ extension domain can comprise a 2′ modification, e.g., a 2-acetylation, e.g., a 2′ methylation, or other modification(s) from Section VIII.

In an embodiment, the 5′ extension domain can comprise as many as 1, 2, 3, 4, 5, 6, 7 or 8 modifications. In an embodiment, the 5′ extension domain comprises as many as 1, 2, 3, or 4 modifications within 5 nucleotides of its 5′ end, e.g., in a modular gRNA molecule. In an embodiment, the 5′ extension domain comprises as many as 1, 2, 3, or 4 modifications within 5 nucleotides of its 3′ end, e.g., in a modular gRNA molecule.

In an embodiment, the 5′ extension domain comprises modifications at two consecutive nucleotides, e.g., two consecutive nucleotides that are within 5 nucleotides of the 5′ end of the 5′ extension domain, within 5 nucleotides of the 3′ end of the 5′ extension domain, or more than 5 nucleotides away from one or both ends of the 5′ extension domain.

In an embodiment, no two consecutive nucleotides are modified within 5 nucleotides of the 5′ end of the 5′ extension domain, within 5 nucleotides of the 3′ end of the 5′ extension domain, or within a region that is more than 5 nucleotides away from one or both ends of the 5′ extension domain. In an embodiment, no nucleotide is modified within 5 nucleotides of the 5′ end of the 5′ extension domain, within 5 nucleotides of the 3′ end of the 5′ extension domain, or within a region that is more than 5 nucleotides away from one or both ends of the 5′ extension domain.

Modifications in the 5′ extension domain can be selected so as to not interfere with gRNA molecule efficacy, which can be evaluated by testing a candidate modification in the system described in Section IV. gRNAs having a candidate 5′ extension domain having a selected length, sequence, degree of complementarity, or degree of modification, can be evaluated in the system described at Section IV. The candidate 5′ extension domain can be placed, either alone, or with one or more other candidate changes in a gRNA molecule/Cas9 molecule system known to be functional with a selected target and evaluated.

In an embodiment, the 5′ extension domain has at least 60, 70, 80, 85, 90 or 95% homology with, or differs by no more than 1, 2, 3, 4, 5, or 6 nucleotides from, a reference 5′ extension domain, e.g., a naturally occurring, e.g., an S. pyogenes, S. aureus or S. thermophilus, 5′ extension domain, or a 5′ extension domain described herein, e.g., from FIGS. 1A-1G.

### The Linking Domain

In a unimolecular gRNA molecule the linking domain is disposed between the first and second complementarity domains. In a modular gRNA molecule, the two molecules are associated with one another by the complementarity domains.

In an embodiment, the linking domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the linking domain is 20+/−10, 30+/−10, 40+/−10, 50+/−10, 60+/−10, 70+/−10, 80+/−10, 90+/−10, or 100+/−10 nucleotides, in length.

In an embodiment, the linking domain is 10 to 100, 10 to 90, 10 to 80, 10 to 70, 10 to 60, 10 to 50, 10 to 40, 10 to 30, 10 to 20 or 10 to 15 nucleotides in length. In other embodiments, the linking domain is 20 to 100, 20 to 90, 20 to 80, 20 to 70, 20 to 60, 20 to 50, 20 to 40, 20 to 30, or 20 to 25 nucleotides in length.

In an embodiment, the linking domain is 1, 2, 3, 4, 5, 6, 7, 8, 9, 10, 11, 12, 13, 14, 15, 16, 17, 18, 19, or 20 nucleotides in length.

In an embodiment, the linking domain is a covalent bond.

In an embodiment, the linking domain comprises a duplexed region, typically adjacent to or within 1, 2, or 3 nucleotides of the 3′ end of the first complementarity domain and/or the 5-end of the second complementarity domain. In an embodiment, the duplexed region can be 20+/−10 base pairs in length. In an embodiment, the duplexed region can be 10+/−5, 15+/−5, 20+/−5, or 30+/−5 base pairs in length. In an embodiment, the duplexed region can be 1, 2, 3, 4, 5, 6, 7, 8, 9, 10, 11, 12, 13, 14, or 15 base pairs in length.

Typically the sequences forming the duplexed region have exact complementarity with one another, though in some embodiments as many as 1, 2, 3, 4, 5, 6, 7 or 8 nucleotides are not complementary with the corresponding nucleotides.

In an embodiment, the linking domain nucleotides do not comprise modifications, e.g., modifications of the type provided in Section VIII. However, in an embodiment, the linking domain comprises one or more modifications, e.g., modifications that it render it less susceptible to degradation or more bio-compatible, e.g., less immunogenic. By way of example, the backbone of the linking domain can be modified with a phosphorothioate, or other modification(s) from Section VIII. In an embodiment a nucleotide of the linking domain can comprise a 2′ modification, e.g., a 2-acetylation, e.g., a 2′ methylation, or other modification(s) from Section VIII. In an embodiment, the linking domain can comprise as many as 1, 2, 3, 4, 5, 6, 7 or 8 modifications.

Modifications in a linking domain can be selected so as to not interfere with targeting efficacy, which can be evaluated by testing a candidate modification in the system described in Section IV. gRNAs having a candidate linking domain having a selected length, sequence, degree of complementarity, or degree of modification, can be evaluated a system described in Section IV. A candidate linking domain can be placed, either alone, or with one or more other candidate changes in a gRNA molecule/Cas9 molecule system known to be functional with a selected target and evaluated.

In an embodiment, the linking domain has at least 60, 70, 80, 85, 90 or 95% homology with, or differs by no more than 1, 2, 3, 4, 5, or 6 nucleotides from, a reference linking domain, e.g., a linking domain described herein, e.g., from FIGS. 1A-1G.

### The Proximal Domain

In an embodiment, the proximal domain is 6+/−2, 7+/−2, 8+/−2, 9+/−2, 10+/−2, 11+/−2, 12+/−2, 13+/−2, 14+/−2, 14+/−2, 16+/−2, 17+/−2, 18+/−2, 19+/−2, or 20+/−2 nucleotides in length.

In an embodiment, the proximal domain is 6, 7, 8, 9, 10, 11, 12, 13, 14, 15, 16, 17, 18, 19, 20, 21, 22, 23, 24, 25 or 26 nucleotides in length.

In an embodiment, the proximal domain is 5 to 20, 7, to 18, 9 to 16, or 10 to 14 nucleotides in length.

In an embodiment, the proximal domain nucleotides do not comprise modifications, e.g., modifications of the type provided in Section VIII. However, in an embodiment, the proximal domain comprises one or more modifications, e.g., modifications that it render it less susceptible to degradation or more bio-compatible, e.g., less immunogenic. By way of example, the backbone of the proximal domain can be modified with a phosphorothioate, or other modification(s) from Section VIII. In an embodiment a nucleotide of the proximal domain can comprise a 2′ modification, e.g., a 2-acetylation, e.g., a 2′ methylation, or other modification(s) from Section VIII.

In an embodiment, the proximal domain can comprise as many as 1, 2, 3, 4, 5, 6, 7 or 8 modifications. In an embodiment, the proximal domain comprises as many as 1, 2, 3, or 4 modifications within 5 nucleotides of its 5′ end, e.g., in a modular gRNA molecule. In an embodiment, the target domain comprises as many as 1, 2, 3, or 4 modifications within 5 nucleotides of its 3′ end, e.g., in a modular gRNA molecule.

In an embodiment, the proximal domain comprises modifications at two consecutive nucleotides, e.g., two consecutive nucleotides that are within 5 nucleotides of the 5′ end of the proximal domain, within 5 nucleotides of the 3′ end of the proximal domain, or more than 5 nucleotides away from one or both ends of the proximal domain. In an embodiment, no two consecutive nucleotides are modified within 5 nucleotides of the 5′ end of the proximal domain, within 5 nucleotides of the 3′ end of the proximal domain, or within a region that is more than 5 nucleotides away from one or both ends of the proximal domain. In an embodiment, no nucleotide is modified within 5 nucleotides of the 5′ end of the proximal domain, within 5 nucleotides of the 3′ end of the proximal domain, or within a region that is more than 5 nucleotides away from one or both ends of the proximal domain.

Modifications in the proximal domain can be selected so as to not interfere with gRNA molecule efficacy, which can be evaluated by testing a candidate modification in the system described in Section IV. gRNAs having a candidate proximal domain having a selected length, sequence, degree of complementarity, or degree of modification, can be evaluated in the system described at Section IV. The candidate proximal domain can be placed, either alone, or with one or more other candidate changes in a gRNA molecule/Cas9 molecule system known to be functional with a selected target and evaluated.

In an embodiment, the proximal domain has at least 60, 70, 80, 85, 90 or 95% homology with, or differs by no more than 1, 2, 3, 4, 5, or 6 nucleotides from, a reference proximal domain, e.g., a naturally occurring, e.g., an S. pyogenes, S. aureus or S. thermophilus, proximal domain, or a proximal domain described herein, e.g., from FIGS. 1A-1G.

### The Tail Domain

In an embodiment, the tail domain is 10+/−5, 20+/−5, 30+/−5, 40+/−5, 50+/−5, 60+/−5, 70+/−5, 80+/−5, 90+/−5, or 100+/−5 nucleotides, in length.

In an embodiment, the tail domain is 20+/−5 nucleotides in length.

In an embodiment, the tail domain is 20+/−10, 30+/−10, 40+/−10, 50+/−10, 60+/−10, 70+/−10, 80+/−10, 90+/−10, or 100+/−10 nucleotides, in length.

In an embodiment, the tail domain is 25+/−10 nucleotides in length.

In an embodiment, the tail domain is 10 to 100, 10 to 90, 10 to 80, 10 to 70, 10 to 60, 10 to 50, 10 to 40, 10 to 30, 10 to 20 or 10 to 15 nucleotides in length.

In other embodiments, the tail domain is 20 to 100, 20 to 90, 20 to 80, 20 to 70, 20 to 60, 20 to 50, 20 to 40, 20 to 30, or 20 to 25 nucleotides in length.

In an embodiment, the tail domain is 1 to 20, 1 to 15, 1 to 10, or 1 to 5 nucleotides in length.

In an embodiment, the tail domain nucleotides do not comprise modifications, e.g., modifications of the type provided in Section VIII. However, in an embodiment, the tail domain comprises one or more modifications, e.g., modifications that it render it less susceptible to degradation or more bio-compatible, e.g., less immunogenic. By way of example, the backbone of the tail domain can be modified with a phosphorothioate, or other modification(s) from Section VIII. In an embodiment a nucleotide of the tail domain can comprise a 2′ modification, e.g., a 2-acetylation, e.g., a 2′ methylation, or other modification(s) from Section VIII.

In some embodiments, the tail domain can have as many as 1, 2, 3, 4, 5, 6, 7 or 8 modifications. In an embodiment, the target domain comprises as many as 1, 2, 3, or 4 modifications within 5 nucleotides of its 5′ end. In an embodiment, the target domain comprises as many as 1, 2, 3, or 4 modifications within 5 nucleotides of its 3′ end.

In an embodiment, the tail domain comprises a tail duplex domain, which can form a tail duplexed region. In an embodiment, the tail duplexed region can be 3, 4, 5, 6, 7, 8, 9, 10, 11, or 12 base pairs in length. In an embodiment, a further single stranded domain, exists 3′ to the tail duplexed domain. In an embodiment, this domain is 3, 4, 5, 6, 7, 8, 9, or 10 nucleotides in length. In an embodiment it is 4 to 6 nucleotides in length.

In an embodiment, the tail domain has at least 60, 70, 80, or 90% homology with, or differs by no more than 1, 2, 3, 4, 5, or 6 nucleotides from, a reference tail domain, e.g., a naturally occurring, e.g., an S. pyogenes, S. aureus or S. thermophilus, tail domain, or a tail domain described herein, e.g., from FIGS. 1A-1G.

In an embodiment, the proximal and tail domain, taken together, comprise the following sequences:

(SEQ ID NO: 33)
AAGGCUAGUCCGUUAUCAACUUGAAAAAGUGGCACCGAGUCGGUGCU,
or
(SEQ ID NO: 34)
AAGGCUAGUCCGUUAUCAACUUGAAAAAGUGGCACCGAGUCGGUGGUGC,
or
(SEQ ID NO: 35)
AAGGCUAGUCCGUUAUCAACUUGAAAAAGUGGCACCGAGUCGGUGCGGAU
C,
or
(SEQ ID NO: 36)
AAGGCUAGUCCGUUAUCAACUUGAAAAAGUG,
or
(SEQ ID NO: 37)
AAGGCUAGUCCGUUAUCA,
or
(SEQ ID NO: 38)
AAGGCUAGUCCG.

In an embodiment, the tail domain comprises the 3′ sequence UUUUUU, e.g., if a U6 promoter is used for transcription.

In an embodiment, the tail domain comprises the 3′ sequence UUUU, e.g., if an H1 promoter is used for transcription.

In an embodiment, tail domain comprises variable numbers of 3′ Us depending, e.g., on the termination signal of the pol-III promoter used.

In an embodiment, the tail domain comprises variable 3′ sequence derived from the DNA template if a T7 promoter is used.

In an embodiment, the tail domain comprises variable 3′ sequence derived from the DNA template, e.g., if in vitro transcription is used to generate the RNA molecule.

In an embodiment, the tail domain comprises variable 3′ sequence derived from the DNA template, e., if a pol-II promoter is used to drive transcription.

Modifications in the tail domain can be selected so as to not interfere with targeting efficacy, which can be evaluated by testing a candidate modification in the system described in Section IV. gRNAs having a candidate tail domain having a selected length, sequence, degree of complementarity, or degree of modification, can be evaluated in the system described in Section IV. The candidate tail domain can be placed, either alone, or with one or more other candidate changes in a gRNA molecule/Cas9 molecule system known to be functional with a selected target and evaluated.

In an embodiment, the tail domain comprises modifications at two consecutive nucleotides, e.g., two consecutive nucleotides that are within 5 nucleotides of the 5′ end of the tail domain, within 5 nucleotides of the 3′ end of the tail domain, or more than 5 nucleotides away from one or both ends of the tail domain. In an embodiment, no two consecutive nucleotides are modified within 5 nucleotides of the 5′ end of the tail domain, within 5 nucleotides of the 3′ end of the tail domain, or within a region that is more than 5 nucleotides away from one or both ends of the tail domain. In an embodiment, no nucleotide is modified within 5 nucleotides of the 5′ end of the tail domain, within 5 nucleotides of the 3′ end of the tail domain, or within a region that is more than 5 nucleotides away from one or both ends of the tail domain.

In an embodiment a gRNA has the following structure:
5′ [targeting domain]-[first complementarity domain]-[linking domain]-[second complementarity domain]-[proximal domain]-[tail domain]-3′
wherein, the targeting domain comprises a core domain and optionally a secondary domain, and is 10 to 50 nucleotides in length;
the first complementarity domain is 5 to 25 nucleotides in length and, In an embodiment has at least 50, 60, 70, 80, 85, 90 or 95% homology with a reference first complementarity domain disclosed herein;
the linking domain is 1 to 5 nucleotides in length;
the second complementarity domain is 5 to 27 nucleotides in length and, in an embodiment has at least 50, 60, 70, 80, 85, 90 or 95% homology with a reference second complementarity domain disclosed herein;
the proximal domain is 5 to 20 nucleotides in length and, in an embodiment has at least 50, 60, 70, 80, 85, 90 or 95% homology with a reference proximal domain disclosed herein; and
the tail domain is absent or a nucleotide sequence is 1 to 50 nucleotides in length and, in an embodiment has at least 50, 60, 70, 80, 85, 90 or 95% homology with a reference tail domain disclosed herein.
   Exemplary Chimeric gRNAs

In an embodiment, a unimolecular, or chimeric, gRNA comprises, preferably from 5′ to 3′:
a targeting domain (which is complementary to a target nucleic acid);
a first complementarity domain, e.g., comprising 15, 16, 17, 18, 19, 20, 21, 22, 23, 24, 25, or 26 nucleotides;
a linking domain;
a second complementarity domain (which is complementary to the first complementarity domain);
a proximal domain; and
a tail domain,
wherein,
(a) the proximal and tail domain, when taken together, comprise at least 15, 18, 20, 25, 30, 31, 35, 40, 45, 49, 50, or 53 nucleotides;
(b) there are at least 15, 18, 20, 25, 30, 31, 35, 40, 45, 49, 50, or 53 nucleotides 3′ to the last nucleotide of the second complementarity domain; or
(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein.

In an embodiment, the proximal and tail domain, when taken together, comprise at least 15, 18, 20, 25, 30, 31, 35, 40, 45, 49, 50, or 53 nucleotides.

In an embodiment, there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length.

In an embodiment, the targeting domain comprises, has, or consists of, 17 nucleotides (e.g., 17 consecutive nucleotides) having complementarity with the target domain, e.g., the targeting domain is 17 nucleotides in length.

In an embodiment, the targeting domain comprises, has, or consists of, 18 nucleotides (e.g., 18 consecutive nucleotides) having complementarity with the target domain, e.g., the targeting domain is 18 nucleotides in length.

In an embodiment, the targeting domain comprises, has, or consists of, 19 nucleotides (e.g., 19 consecutive nucleotides) having complementarity with the target domain, e.g., the targeting domain is 19 nucleotides in length.

In an embodiment, the targeting domain comprises, has, or consists of, 20 nucleotides (e.g., 20 consecutive nucleotides) having complementarity with the target domain, e.g., the targeting domain is 20 nucleotides in length.

In an embodiment, the targeting domain comprises, has, or consists of, 21 nucleotides (e.g., 21 consecutive nucleotides) having complementarity with the target domain, e.g., the targeting domain is 21 nucleotides in length.

In an embodiment, the targeting domain comprises, has, or consists of, 22 nucleotides (e.g., 22 consecutive nucleotides) having complementarity with the target domain, e.g., the targeting domain is 22 nucleotides in length.

In an embodiment, the targeting domain comprises, has, or consists of, 23 nucleotides (e.g., 23 consecutive nucleotides) having complementarity with the target domain, e.g., the targeting domain is 23 nucleotides in length.

In an embodiment, the targeting domain comprises, has, or consists of, 24 nucleotides (e.g., 24 consecutive nucleotides) having complementarity with the target domain, e.g., the targeting domain is 24 nucleotides in length.

In an embodiment, the targeting domain comprises, has, or consists of, 25 nucleotides (e.g., 25 consecutive nucleotides) having complementarity with the target domain, e.g., the targeting domain is 25 nucleotides in length.

In an embodiment, the targeting domain comprises, has, or consists of, 26 nucleotides (e.g., 26 consecutive nucleotides) having complementarity with the target domain, e.g., the targeting domain is 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number:
NNNNNNNNNNNNNNNNGUUUUAGAGCUAGAAAUAGCAAGUUAAAAUAA GGCUAGUCCGUUAUCAACUUGAAAAAGUGGCACCGAGUCGGUGCUUUUUU
   (SEQ ID NO: 45). In an embodiment, the unimolecular, or chimeric, gRNA molecule is a S. pyogenes gRNA molecule.

In some embodiments, the unimolecular, or chimeric, gRNA molecule (comprising a targeting domain, a first complementary domain, a linking domain, a second complementary domain, a proximal domain and, optionally, a tail domain) comprises the following sequence in which the targeting domain is depicted as 20 Ns but could be any sequence and range in length from 16 to 26 nucleotides and in which the gRNA sequence is followed by 6 Us, which serve as a termination signal for the U6 promoter, but which could be either absent or fewer in number:
NNNNNNNNNNNNNNNNNNGUUUUAGUACUCUGGAAACAGAAUCUACUAAA ACAAGGCAAAAUGCCGUGUUUAUCUCGUCAACUUGUUGGCGAGAUUUUUU
   (SEQ ID NO: 40). In an embodiment, the unimolecular, or chimeric, gRNA molecule is a S. aureus gRNA molecule.
   Exemplary Modular gRNAs

In an embodiment, a modular gRNA comprises:
a first strand comprising, preferably from 5′ to 3′;
   a targeting domain, e.g., comprising 15, 16, 17, 18, 19, 20, 21, 22, 23, 24, 25, or 26 nucleotides;
   a first complementarity domain; and
   a second strand, comprising, preferably from 5′ to 3′:
      optionally a 5′ extension domain;
   a second complementarity domain;
   a proximal domain; and
   a tail domain,
wherein:
   (a) the proximal and tail domain, when taken together, comprise at least 15, 18, 20, 25, 30, 31, 35, 40, 45, 49, 50, or 53 nucleotides;
(b) there are at least 15, 18, 20, 25, 30, 31, 35, 40, 45, 49, 50, or 53 nucleotides 3′ to the last nucleotide of the second complementarity domain; or
(c) there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the sequence from (a), (b), or (c), has at least 60, 75, 80, 85, 90, 95, or 99% homology with the corresponding sequence of a naturally occurring gRNA, or with a gRNA described herein.

In an embodiment, the proximal and tail domain, when taken together, comprise at least 15, 18, 20, 25, 30, 31, 35, 40, 45, 49, 50, or 53 nucleotides.

In an embodiment there are at least 15, 18, 20, 25, 30, 31, 35, 40, 45, 49, 50, or 53 nucleotides 3′ to the last nucleotide of the second complementarity domain.

In an embodiment, there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 16, 17, 18, 19, 20, 21, 22, 23, 24, 25 or 26 nucleotides (e.g., 16, 17, 18, 19, 20, 21, 22, 23, 24, 25 or 26 consecutive nucleotides) having complementarity with the target domain, e.g., the targeting domain is 16, 17, 18, 19, 20, 21, 22, 23, 24, 25 or 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length.

In an embodiment, the targeting domain comprises, has, or consists of, 17 nucleotides (e.g., 17 consecutive nucleotides) having complementarity with the target domain, e.g., the targeting domain is 17 nucleotides in length.

In an embodiment, the targeting domain comprises, has, or consists of, 18 nucleotides (e.g., 18 consecutive nucleotides) having complementarity with the target domain, e.g., the targeting domain is 18 nucleotides in length.

In an embodiment, the targeting domain comprises, has, or consists of, 19 nucleotides (e.g., 19 consecutive nucleotides) having complementarity with the target domain, e.g., the targeting domain is 19 nucleotides in length.

In an embodiment, the targeting domain comprises, has, or consists of, 20 nucleotides (e.g., 20 consecutive nucleotides) having complementarity with the target domain, e.g., the targeting domain is 20 nucleotides in length.

In an embodiment, the targeting domain comprises, has, or consists of, 21 nucleotides (e.g., 21 consecutive nucleotides) having complementarity with the target domain, e.g., the targeting domain is 21 nucleotides in length.

In an embodiment, the targeting domain comprises, has, or consists of, 22 nucleotides (e.g., 22 consecutive nucleotides) having complementarity with the target domain, e.g., the targeting domain is 22 nucleotides in length.

In an embodiment, the targeting domain comprises, has, or consists of, 23 nucleotides (e.g., 23 consecutive nucleotides) having complementarity with the target domain, e.g., the targeting domain is 23 nucleotides in length.

In an embodiment, the targeting domain comprises, has, or consists of, 24 nucleotides (e.g., 24 consecutive nucleotides) having complementarity with the target domain, e.g., the targeting domain is 24 nucleotides in length.

In an embodiment, the targeting domain comprises, has, or consists of, 25 nucleotides (e.g., 25 consecutive nucleotides) having complementarity with the target domain, e.g., the targeting domain is 25 nucleotides in length.

In an embodiment, the targeting domain comprises, has, or consists of, 26 nucleotides (e.g., 26 consecutive nucleotides) having complementarity with the target domain, e.g., the targeting domain is 26 nucleotides in length.

In an embodiment, the targeting domain comprises, has, or consists of, 16 nucleotides (e.g., 16 consecutive nucleotides) having complementarity with the target domain, e.g., the targeting domain is 16 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 16 nucleotides (e.g., 16 consecutive nucleotides) having complementarity with the target domain, e.g., the targeting domain is 16 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 17 nucleotides (e.g., 17 consecutive nucleotides) having complementarity with the target domain, e.g., the targeting domain is 17 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 18 nucleotides (e.g., 18 consecutive nucleotides) having complementarity with the target domain, e.g., the targeting domain is 18 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 19 nucleotides (e.g., 19 consecutive nucleotides) having complementarity with the target domain, e.g., the targeting domain is 19 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 20 nucleotides (e.g., 20 consecutive nucleotides) having complementarity with the target domain, e.g., the targeting domain is 20 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 21 nucleotides (e.g., 21 consecutive nucleotides) having complementarity with the target domain, e.g., the targeting domain is 21 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 22 nucleotides (e.g., 22 consecutive nucleotides) having complementarity with the target domain, e.g., the targeting domain is 22 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 23 nucleotides (e.g., 23 consecutive nucleotides) having complementarity with the target domain, e.g., the targeting domain is 23 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 24 nucleotides (e.g., 24 consecutive nucleotides) having complementarity with the target domain, e.g., the targeting domain is 24 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 25 nucleotides (e.g., 25 consecutive nucleotides) having complementarity with the target domain, e.g., the targeting domain is 25 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and the proximal and tail domain, when taken together, comprise at least 15, 18, 20, 25, 30, 31, 35, 40, 45, 49, 50, or 53 nucleotides.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 15, 18, 20, 25, 30, 31, 35, 40, 45, 49, 50, or 53 nucleotides 3′ to the last nucleotide of the second complementarity domain.

In an embodiment, the targeting domain comprises, has, or consists of, 26 nucleotides (e.g., 26 consecutive nucleotides) having complementarity with the target domain, e.g., the targeting domain is 26 nucleotides in length; and there are at least 16, 19, 21, 26, 31, 32, 36, 41, 46, 50, 51, or 54 nucleotides 3′ to the last nucleotide of the second complementarity domain that is complementary to its corresponding nucleotide of the first complementarity domain.

I. Methods for Designing gRNAs

Methods for designing gRNAs are described herein, including methods for selecting, designing and validating target domains. Exemplary targeting domains are also provided herein. Targeting domains discussed herein can be incorporated into the gRNAs described herein.

Methods for selection and validation of target sequences as well as off-target analyses are described, e.g., in Mali et al., 2013 SCIENCE 339(6121): 823-826; Hsu et al. NAT BIOTECHNOL, 31(9): 827-32; Fu et al., 2014 NAT BIOTECHNOL, doi: 10.1038/nbt.2808. PubMed PMID: 24463574; Heigwer et al., 2014 NAT METHODS 11(2):122-3. doi: 10.1038/nmeth.2812. PubMed PMID: 24481216; Bae et al., 2014 BIOINFORMATICS PubMed PMID: 24463181; Xiao A et al., 2014 BIOINFORMATICS PubMed PMID: 24389662.

For example, a software tool can be used to optimize the choice of gRNA within a user's target sequence, e.g., to minimize total off-target activity across the genome. Off target activity may be other than cleavage. For each possible gRNA choice using S. pyogenes Cas9, the tool can identify all off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA is then ranked according to its total predicted off-target cleavage; the top-ranked gRNAs represent those that are likely to have the greatest on-target and the least off-target cleavage. Other functions, e.g., automated reagent design for CRISPR construction, primer design for the on-target Surveyor assay, and primer design for high-throughput detection and quantification of off-target cleavage via next-gen sequencing, can also be included in the tool. Candidate gRNA molecules can be evaluated by art-known methods or as described in Section IV herein.

The Targeting domains discussed herein can be incorporated into the gRNAs described herein.

Strategies to Identify gRNAs for S. pyogenes, S. Aureus, and N. meningitidis to Knock Out the UL19 Gene

As an example, three strategies were utilized to identify gRNAs for use with S. pyogenes, S. aureus and N. meningitidis Cas9 enzymes.

In the first strategy, guide RNAs (gRNAs) for use with the S. pyogenes (Tables 1A-1C) Cas9 were identified using the publically available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available Repeat-Masker program. RepeatMmasker searches input DNA sequences for repeated elements and regions of low complexity. The output is a detailed annotation of the repeats present in a given query sequence. Following identification, gRNAs for use with a S. pyogenes Cas9 were ranked into 3 tiers.

The gRNAs in tier 1 were selected based on their distance to the target site and their orthogonality in the genome (based on the ZiFiT identification of close matches in the human genome containing an NGG PAM). As an example, for all targets, both 17-mer and 20-mer gRNAs were designed. gRNAs were also selected both for single-gRNA nuclease cutting and for the dual gRNA nickase strategy. Criteria for selecting gRNAs and the determination for which gRNAs can be used for which strategy is based on several considerations:
1. For the dual nickase strategy, gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, it will also often result in indel mutations at the site of only one of the gRNAs. Candidate pair members can be tested for how efficiently they remove the entire sequence versus just causing indel mutations at the site of one gRNA.

While it can be desirable to have gRNAs start with a 5′ G, this requirement was relaxed for some gRNAs in tier 1 in order to identify guides in the correct orientation, within a reasonable distance to the target position (i.e., within the first 500 bp of the coding sequence) and with a high level of orthogonality against the human genome. In order to find a pair for the dual-nickase strategy it was necessary to either extend the distance from the target position or remove the requirement for the 5′G. Tier 2 gRNAs were selected based on location within the first 500 bp of the coding sequence in the HSV gene. Tier 3 gRNAs were selected based on their location in the coding sequence, but downstream of the first 500 bp of the HSV gene. Note that tiers are non-inclusive (each gRNA is listed only once). In certain instances, no gRNA was identified based on the criteria of the particular tier.

As discussed above, gRNAs were identified for single-gRNA nuclease cleavage as well as for a dual-gRNA paired “nickase” strategy, as indicated.

gRNAs for use with the N. meningitidis (Tables 1F-1G) and S. aureus (Tables 1D-1E) Cas9s were identified manually by scanning genomic DNA sequence for the presence of PAM sequences. These gRNAs were separated into two tiers for each species. The first tier includes gRNAs selected based on location in the first 500 bp of the coding sequence of the HSV gene. The second tier includes gRNAs selected based on location in the coding sequence, but downstream of the first 500 bp of the HSV gene.

In a second strategy, Guide RNAs (gRNAs) for use with S. pyogenes, S. aureus and N. meningitidis Cas9s were identified using a DNA sequence searching algorithm. Guide RNA design was carried out using a custom guide RNA design software based on the public tool cas-offinder (reference:Cas-OFFinder: a fast and versatile algorithm that searches for potential off-target sites of Cas9 RNA-guided endonucleases., Bioinformatics. 2014 Feb. 17. Bae S, Park J, Kim J S. PMID: 24463181). Said custom guide RNA design software scores guides after calculating their genomewide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, gRNAs were ranked into tiers based on their distance to the target site, their orthogonality and presence of a 5′ G (based on identification of close matches in the human genome containing a relevant PAM (e.g., in the case of S. pyogenes, a NGG PAM, in the case of S. aureus, a NNGRRT or NNGRRV PAM, and in the case of N. meningitidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer gRNAs that have no identical sequences in the human genome besides the intended target, nor any sequences that contain one or two mismatches in the target sequence. Targeting domains with good orthogonality are selected to minimize off-target DNA cleavage.

As an example, for S. pyogenes and N. meningitidis targets, 17-mer, or 20-mer gRNAs were designed. As another example, for S. aureus targets, 18-mer, 19-mer, 20-mer, 21-mer, 22-mer, 23-mer and 24-mer gRNAs were designed. Targeting domains, disclosed herein, may comprise the 17-mer described in Tables 5A-5E, 6A-6G or 7A-7D, e.g., the targeting domains of 18 or more nucleotides may comprise the 17-mer gRNAs described in Tables 5A-5E, 6A-6G or 7A-7D. Targeting domains, disclosed herein, may comprises the 18-mer described in Tables 5A-5E, 6A-6G or 7A-7D, e.g., the targeting domains of 19 or more nucleotides may comprise the 18-mer gRNAs described in Tables 5A-5E, 6A-6G or 7A-7D. Targeting domains, disclosed herein, may comprises the 19-mer described in Tables 5A-5E, 6A-6G or 7A-7D, e.g., the targeting domains of 20 or more nucleotides may comprise the 19-mer gRNAs described in Tables 5A-5E, 6A-6G or 7A-7D. Targeting domains, disclosed herein, may comprises the 20-mer gRNAs described in Tables 5A-5E, 6A-6G or 7A-7D, e.g., the targeting domains of 21 or more nucleotides may comprise the 20-mer gRNAs described in Tables 5A-5E, 6A-6G or 7A-7D. Targeting domains, disclosed herein, may comprises the 21-mer described in Tables 5A-5E, 6A-6G or 7A-7D, e.g., the targeting domains of 22 or more nucleotides may comprise the 21-mer gRNAs described in Tables 5A-5E, 6A-6G or 7A-7D. Targeting domains, disclosed herein, may comprises the 22-mer described in Tables 5A-5E, 6A-6G or 7A-7D, e.g., the targeting domains of 23 or more nucleotides may comprise the 22-mer gRNAs described in Tables 5A-5E, 6A-6G or 7A-7D. Targeting domains, disclosed herein, may comprises the 23-mer described in Tables 5A-5E, 6A-6G or 7A-7D, e.g., the targeting domains of 24 or more nucleotides may comprise the 23-mer gRNAs described in Tables 5A-5E, 6A-6G or 7A-7D. Targeting domains, disclosed herein, may comprises the 24-mer described in Tables 5A-5E, 6A-6G or 7A-7D, e.g., the targeting domains of 25 or more nucleotides may comprise the 24-mer gRNAs described in Tables 5A-5E, 6A-6G or 7A-7D. gRNAs were identified for both single-gRNA nuclease cleavage and for a dual-gRNA paired “nickase” strategy. Criteria for selecting gRNAs and the determination for which gRNAs can be used for the dual-gRNA paired “nickase” strategy is based on two considerations:
1. gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the site of one gRNA.

The targeting domains discussed herein can be incorporated into the gRNAs described herein.

gRNAs were identified and ranked into 5 tiers for S. pyogenes (Tables 5A-5E), and N. meningitidis (Tables 7A-7D); and 7 tiers for S. aureus (Tables 6A-6G). For S. pyogenes, and N. meningitidis, the targeting domain for tier 1 gRNA molecules were selected based on (1) distance to a target site (e.g., start codon), e.g., within 500 bp (e.g., downstream) of the target site (e.g., start codon), (2) a high level of orthogonality and (3) the presence of 5′G. The targeting domain for tier 2 gRNA molecules were selected based on (1) distance to a target site (e.g., start codon), e.g., within 500 bp (e.g., downstream) of the target site (e.g., start codon) and (2) a high level of orthogonality. The targeting domain for tier 3 gRNA molecules were selected based on (1) distance to a target site (e.g., start codon), e.g., within 500 bp (e.g., downstream) of the target site (e.g., start codon) and (2) the presence of 5′G. The targeting domain for tier 4 gRNA molecules were selected based on distance to a target site (e.g., start codon), e.g., within 500 bp (e.g., downstream) of the target site (e.g., start codon). The targeting domain for tier 5 gRNA molecules were selected based on distance to the target site (e.g., start codon), e.g., within reminder of the coding sequence, e.g., downstream of the first 500 bp of coding sequence (e.g., anywhere from +500 (relative to the start codon) to the stop codon). For S. aureus, the targeting domain for tier 1 gRNA molecules were selected based on (1) distance to a target site (e.g., start codon), e.g., within 500 bp (e.g., downstream) of the target site (e.g., start codon), (2) a high level of orthogonality, (3) the presence of 5′G and (4) PAM is NNGRRT. The targeting domain for tier 2 gRNA molecules were selected based on (1) distance to a target site (e.g., start codon), e.g., within 500 bp (e.g., downstream) of the target site (e.g., start codon), (2) a high level of orthogonality, and (3) PAM is NNGRRT. The targeting domain for tier 3 gRNA molecules were selected based on (1) distance to a target site (e.g., start codon), e.g., within 500 bp (e.g., downstream) of the target site (e.g., start codon) and (2) PAM is NNGRRT. The targeting domain for tier 4 gRNA molecules were selected based on (1) distance to a target site (e.g., start codon), e.g., within 500 bp (e.g., downstream) of the target site (e.g., start codon) and (2) PAM is NNGRRV. The targeting domain for tier 5 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon), (2) the presence of 5′G and (3) PAM is NNGRRT. The targeting domain for tier 6 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRT. The targeting domain for tier 7 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRV. Note that tiers are non-inclusive (each gRNA is listed only once for the strategy). In certain instances, no gRNA was identified based on the criteria of the particular tier.

Strategies to Identify gRNAs for S. pyogenes, S. Aureus, and N. meningitidis to Knock Out the UL30 Gene

As an example, three strategies were utilized to identify gRNAs for use with S. pyogenes, S. aureus and N. meningitidis Cas9 enzymes.

In the first strategy, guide RNAs (gRNAs) for use with the S. pyogenes (Tables 2A-2C) Cas9 were identified using the publically available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available Repeat-Masker program. RepeatMmasker searches input DNA sequences for repeated elements and regions of low complexity. The output is a detailed annotation of the repeats present in a given query sequence. Following identification, gRNAs for use with a S. pyogenes Cas9 were ranked into 3 tiers.

The gRNAs in tier 1 were selected based on their distance to the target site and their orthogonality in the genome (based on the ZiFiT identification of close matches in the human genome containing an NGG PAM). As an example, for all targets, both 17-mer and 20-mer gRNAs were designed. gRNAs were also selected both for single-gRNA nuclease cutting and for the dual gRNA nickase strategy. Criteria for selecting gRNAs and the determination for which gRNAs can be used for which strategy is based on several considerations:
1. For the dual nickase strategy, gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, it will also often result in indel mutations at the site of only one of the gRNAs. Candidate pair members can be tested for how efficiently they remove the entire sequence versus just causing indel mutations at the site of one gRNA.

While it can be desirable to have gRNAs start with a 5′ G, this requirement was relaxed for some gRNAs in tier 1 in order to identify guides in the correct orientation, within a reasonable distance to the target position (i.e., within the first 500 bp of the coding sequence) and with a high level of orthogonality against the human genome. In order to find a pair for the dual-nickase strategy it was necessary to either extend the distance from the target position or remove the requirement for the 5′G. Tier 2 gRNAs were selected based on location within the first 500 bp of the coding sequence in the HSV gene. Tier 3 gRNAs were selected based on their location in the coding sequence, but downstream of the first 500 bp of the HSV gene. Note that tiers are non-inclusive (each gRNA is listed only once). In certain instances, no gRNA was identified based on the criteria of the particular tier.

As discussed above, gRNAs were identified for single-gRNA nuclease cleavage as well as for a dual-gRNA paired “nickase” strategy, as indicated. gRNAs for use with the N. meningitidis (Tables 2F-2G) and S. aureus (Tables 2D-2E) Cas9s were identified manually by scanning genomic DNA sequence for the presence of PAM sequences. These gRNAs were separated into two tiers for each species. The first tier includes gRNAs selected based on location in the first 500 bp of the coding sequence of the HSV gene. The second tier includes gRNAs selected based on location in the coding sequence, but downstream of the first 500 bp of the HSV gene.

In a second strategy, Guide RNAs (gRNAs) for use with S. pyogenes, S. aureus and N. meningitidis Cas9s were identified using a DNA sequence searching algorithm. Guide RNA design was carried out using a custom guide RNA design software based on the public tool cas-offinder (reference:Cas-OFFinder: a fast and versatile algorithm that searches for potential off-target sites of Cas9 RNA-guided endonucleases., Bioinformatics. 2014 Feb. 17. Bae S, Park J, Kim J S. PMID: 24463181). Said custom guide RNA design software scores guides after calculating their genome wide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, gRNAs were ranked into tiers based on their distance to the target site, their orthogonality and presence of a 5′ G (based on identification of close matches in the human genome containing a relevant PAM (e.g., in the case of S. pyogenes, a NGG PAM, in the case of S. aureus, a NNGRRT or NNGRRV PAM, and in the case of N. meningitidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer gRNAs that have no identical sequences in the human genome besides the intended target, nor any sequences that contain one or two mismatches in the target sequence. Targeting domains with good orthogonality are selected to minimize off-target DNA cleavage.

As an example, for S. pyogenes and N. meningitidis targets, 17-mer, or 20-mer gRNAs were designed. As another example, for S. aureus targets, 18-mer, 19-mer, 20-mer, 21-mer, 22-mer, 23-mer and 24-mer gRNAs were designed. Targeting domains, disclosed herein, may comprise the 17-mer described in Tables 8A-8E, 9A-9G or 10A-10C, e.g., the targeting domains of 18 or more nucleotides may comprise the 17-mer gRNAs described in Tables 8A-8E, 9A-9G or 10A-10C. Targeting domains, disclosed herein, may comprises the 18-mer described in Tables 8A-8E, 9A-9G or 10A-10C, e.g., the targeting domains of 19 or more nucleotides may comprise the 18-mer gRNAs described in Tables 8A-8E, 9A-9G or 10A-10C. Targeting domains, disclosed herein, may comprises the 19-mer described in Tables 8A-8E, 9A-9G or 10A-10C, e.g., the targeting domains of 20 or more nucleotides may comprise the 19-mer gRNAs described in Tables 8A-8E, 9A-9G or 10A-10C. Targeting domains, disclosed herein, may comprises the 20-mer gRNAs described in Tables 8A-8E, 9A-9G or 10A-10C, e.g., the targeting domains of 21 or more nucleotides may comprise the 20-mer gRNAs described in Tables 8A-8E, 9A-9G or 10A-10C. Targeting domains, disclosed herein, may comprises the 21-mer described in Tables 8A-8E, 9A-9G or 10A-10C, e.g., the targeting domains of 22 or more nucleotides may comprise the 21-mer gRNAs described in Tables 8A-8E, 9A-9G or 10A-10C. Targeting domains, disclosed herein, may comprises the 22-mer described in Tables 8A-8E, 9A-9G or 10A-10C, e.g., the targeting domains of 23 or more nucleotides may comprise the 22-mer gRNAs described in Tables 8A-8E, 9A-9G or 10A-10C. Targeting domains, disclosed herein, may comprises the 23-mer described in Tables 8A-8E, 9A-9G or 10A-10C, e.g., the targeting domains of 24 or more nucleotides may comprise the 23-mer gRNAs described in Tables 8A-8E, 9A-9G or 10A-10C. Targeting domains, disclosed herein, may comprises the 24-mer described in Tables 8A-8E, 9A-9G or 10A-10C, e.g., the targeting domains of 25 or more nucleotides may comprise the 24-mer gRNAs described in Tables 8A-8E, 9A-9G or 10A-10C. gRNAs were identified for both single-gRNA nuclease cleavage and for a dual-gRNA paired “nickase” strategy. Criteria for selecting gRNAs and the determination for which gRNAs can be used for the dual-gRNA paired “nickase” strategy is based on two considerations:
1. gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the site of one gRNA.

The targeting domains discussed herein can be incorporated into the gRNAs described herein.

gRNAs were identified and ranked into 5 tiers for S. pyogenes (Tables 8A-8E), and N. meningitidis (Tables 10A-10C); and 7 tiers for S. aureus (Tables 9A-9G). For S. pyogenes, and N. meningitidis, the targeting domain for tier 1 gRNA molecules were selected based on (1) distance to a target site (e.g., start codon), e.g., within 500 bp (e.g., downstream) of the target site (e.g., start codon), (2) a high level of orthogonality and (3) the presence of 5′G. The targeting domain for tier 2 gRNA molecules were selected based on (1) distance to a target site (e.g., start codon), e.g., within 500 bp (e.g., downstream) of the target site (e.g., start codon) and (2) a high level of orthogonality. The targeting domain for tier 3 gRNA molecules were selected based on (1) distance to a target site (e.g., start codon), e.g., within 500 bp (e.g., downstream) of the target site (e.g., start codon) and (2) the presence of 5′G. The targeting domain for tier 4 gRNA molecules were selected based on distance to a target site (e.g., start codon), e.g., within 500 bp (e.g., downstream) of the target site (e.g., start codon). The targeting domain for tier 5 gRNA molecules were selected based on distance to the target site (e.g., start codon), e.g., within reminder of the coding sequence, e.g., downstream of the first 500 bp of coding sequence (e.g., anywhere from +500 (relative to the start codon) to the stop codon). For S. aureus, the targeting domain for tier 1 gRNA molecules were selected based on (1) distance to a target site (e.g., start codon), e.g., within 500 bp (e.g., downstream) of the target site (e.g., start codon), (2) a high level of orthogonality, (3) the presence of 5′G and (4) PAM is NNGRRT. The targeting domain for tier 2 gRNA molecules were selected based on (1) distance to a target site (e.g., start codon), e.g., within 500 bp (e.g., downstream) of the target site (e.g., start codon), (2) a high level of orthogonality, and (3) PAM is NNGRRT. The targeting domain for tier 3 gRNA molecules were selected based on (1) distance to a target site (e.g., start codon), e.g., within 500 bp (e.g., downstream) of the target site (e.g., start codon) and (2) PAM is NNGRRT. The targeting domain for tier 4 gRNA molecules were selected based on (1) distance to a target site (e.g., start codon), e.g., within 500 bp (e.g., downstream) of the target site (e.g., start codon) and (2) PAM is NNGRRV. The targeting domain for tier 5 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon), (2) the presence of 5′G and (3) PAM is NNGRRT. The targeting domain for tier 6 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRT. The targeting domain for tier 7 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRV. Note that tiers are non-inclusive (each gRNA is listed only once for the strategy). In certain instances, no gRNA was identified based on the criteria of the particular tier.

Strategies to Identify gRNAs for S. pyogenes, S. Aureus, and N. meningitidis to Knock Out The UL48 gene

As an example, three strategies were utilized to identify gRNAs for use with S. pyogenes, S. aureus and N. meningitidis Cas9 enzymes.

In the first strategy, guide RNAs (gRNAs) for use with the S. pyogenes (Tables 3A-3C) Cas9 were identified using the publically available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available Repeat-Masker program. RepeatMmasker searches input DNA sequences for repeated elements and regions of low complexity. The output is a detailed annotation of the repeats present in a given query sequence. Following identification, gRNAs for use with a S. pyogenes Cas9 were ranked into 3 tiers.

The gRNAs in tier 1 were selected based on their distance to the target site and their orthogonality in the genome (based on the ZiFiT identification of close matches in the human genome containing an NGG PAM). As an example, for all targets, both 17-mer and 20-mer gRNAs were designed. gRNAs were also selected both for single-gRNA nuclease cutting and for the dual gRNA nickase strategy. Criteria for selecting gRNAs and the determination for which gRNAs can be used for which strategy is based on several considerations:
1. For the dual nickase strategy, gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, it will also often result in indel mutations at the site of only one of the gRNAs. Candidate pair members can be tested for how efficiently they remove the entire sequence versus just causing indel mutations at the site of one gRNA.

While it can be desirable to have gRNAs start with a 5′ G, this requirement was relaxed for some gRNAs in tier 1 in order to identify guides in the correct orientation, within a reasonable distance to the target position (i.e., within the first 500 bp of the coding sequence) and with a high level of orthogonality against the human genome. In order to find a pair for the dual-nickase strategy it was necessary to either extend the distance from the target position or remove the requirement for the 5′G. Tier 2 gRNAs were selected based on location within the first 500 bp of the coding sequence in the HSV gene. Tier 3 gRNAs were selected based on their location in the coding sequence, but downstream of the first 500 bp of the HSV gene. Note that tiers are non-inclusive (each gRNA is listed only once). In certain instances, no gRNA was identified based on the criteria of the particular tier.

As discussed above, gRNAs were identified for single-gRNA nuclease cleavage as well as for a dual-gRNA paired “nickase” strategy, as indicated.

gRNAs for use with the N. meningitidis (Tables 3F-3G) and S. aureus (Tables 3D-3E) Cas9s were identified manually by scanning genomic DNA sequence for the presence of PAM sequences. These gRNAs were separated into two tiers for each species. The first tier includes gRNAs selected based on location in the first 500 bp of the coding sequence of the HSV gene. The second tier includes gRNAs selected based on location in the coding sequence, but downstream of the first 500 bp of the HSV gene.

In a second strategy, Guide RNAs (gRNAs) for use with S. pyogenes, S. aureus and N. meningitidis Cas9s were identified using a DNA sequence searching algorithm. Guide RNA design was carried out using a custom guide RNA design software based on the public tool cas-offinder (reference:Cas-OFFinder: a fast and versatile algorithm that searches for potential off-target sites of Cas9 RNA-guided endonucleases., Bioinformatics. 2014 Feb. 17.

Bae S, Park J, Kim J S. PMID: 24463181). Said custom guide RNA design software scores guides after calculating their genomewide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, gRNAs were ranked into tiers based on their distance to the target site, their orthogonality and presence of a 5′ G (based on identification of close matches in the human genome containing a relavant PAM (e.g., in the case of S. pyogenes, a NGG PAM, in the case of S. aureus, a NNGRRT or NNGRRV PAM, and in the case of N. meningitidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer gRNAs that have no identical sequences in the human genome besides the intended target, nor any sequences that contain one or two mismatches in the target sequence. Targeting domains with good orthogonality are selected to minimize off-target DNA cleavage.

As an example, for S. pyogenes and N. meningitidis targets, 17-mer, or 20-mer gRNAs were designed. As another example, for S. aureus targets, 18-mer, 19-mer, 20-mer, 21-mer, 22-mer, 23-mer and 24-mer gRNAs were designed. Targeting domains, disclosed herein, may comprise the 17-mer described in Tables 11A-11E, 12A-12G or 13A-13C, e.g., the targeting domains of 18 or more nucleotides may comprise the 17-mer gRNAs described in Tables 11A-11E, 12A-12G or 13A-13C. Targeting domains, disclosed herein, may comprises the 18-mer described in Tables 11A-11E, 12A-12G or 13A-13C, e.g., the targeting domains of 19 or more nucleotides may comprise the 18-mer gRNAs described in Tables 11A-11E, 12A-12G or 13A-13C. Targeting domains, disclosed herein, may comprises the 19-mer described in Tables 11A-11E, 12A-12G or 13A-13C, e.g., the targeting domains of 20 or more nucleotides may comprise the 19-mer gRNAs described in Tables 11A-11E, 12A-12G or 13A-13C. Targeting domains, disclosed herein, may comprises the 20-mer gRNAs described in Tables 11A-11E, 12A-12G or 13A-13C, e.g., the targeting domains of 21 or more nucleotides may comprise the 20-mer gRNAs described in Tables 11A-11E, 12A-12G or 13A-13C. Targeting domains, disclosed herein, may comprises the 21-mer described in Tables 11A-11E, 12A-12G or 13A-13C, e.g., the targeting domains of 22 or more nucleotides may comprise the 21-mer gRNAs described in Tables 11A-11E, 12A-12G or 13A-13C. Targeting domains, disclosed herein, may comprises the 22-mer described in Tables 11A-11E, 12A-12G or 13A-13C, e.g., the targeting domains of 23 or more nucleotides may comprise the 22-mer gRNAs described in Tables 11A-11E, 12A-12G or 13A-13C. Targeting domains, disclosed herein, may comprises the 23-mer described in Tables 11A-11E, 12A-12G or 13A-13C, e.g., the targeting domains of 24 or more nucleotides may comprise the 23-mer gRNAs described in Tables 11A-11E, 12A-12G or 13A-13C. Targeting domains, disclosed herein, may comprises the 24-mer described in Tables 11A-11E, 12A-12G or 13A-13C, e.g., the targeting domains of 25 or more nucleotides may comprise the 24-mer gRNAs described in Tables 11A-11E, 12A-12G or 13A-13C.gRNAs were identified for both single-gRNA nuclease cleavage and for a dual-gRNA paired “nickase” strategy. Criteria for selecting gRNAs and the determination for which gRNAs can be used for the dual-gRNA paired “nickase” strategy is based on two considerations:
1. gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the site of one gRNA.

The targeting domains discussed herein can be incorporated into the gRNAs described herein.

gRNAs were identified and ranked into 5 tiers for S. pyogenes (Tables 11A-11E), and N. meningitidis (Tables 13A-13C); and 7 tiers for S. aureus (Tables 12A-12G). For S. pyogenes, and N. meningitidis, the targeting domain for tier 1 gRNA molecules were selected based on (1) distance to a target site (e.g., start codon), e.g., within 500 bp (e.g., downstream) of the target site (e.g., start codon), (2) a high level of orthogonality and (3) the presence of 5′G. The targeting domain for tier 2 gRNA molecules were selected based on (1) distance to a target site (e.g., start codon), e.g., within 500 bp (e.g., downstream) of the target site (e.g., start codon) and (2) a high level of orthogonality. The targeting domain for tier 3 gRNA molecules were selected based on (1) distance to a target site (e.g., start codon), e.g., within 500 bp (e.g., downstream) of the target site (e.g., start codon) and (2) the presence of 5′G. The targeting domain for tier 4 gRNA molecules were selected based on distance to a target site (e.g., start codon), e.g., within 500 bp (e.g., downstream) of the target site (e.g., start codon). The targeting domain for tier 5 gRNA molecules were selected based on distance to the target site (e.g., start codon), e.g., within reminder of the coding sequence, e.g., downstream of the first 500 bp of coding sequence (e.g., anywhere from +500 (relative to the start codon) to the stop codon). For S. aureus, the targeting domain for tier 1 gRNA molecules were selected based on (1) distance to a target site (e.g., start codon), e.g., within 500 bp (e.g., downstream) of the target site (e.g., start codon), (2) a high level of orthogonality, (3) the presence of 5′G and (4) PAM is NNGRRT. The targeting domain for tier 2 gRNA molecules were selected based on (1) distance to a target site (e.g., start codon), e.g., within 500 bp (e.g., downstream) of the target site (e.g., start codon), (2) a high level of orthogonality, and (3) PAM is NNGRRT. The targeting domain for tier 3 gRNA molecules were selected based on (1) distance to a target site (e.g., start codon), e.g., within 500 bp (e.g., downstream) of the target site (e.g., start codon) and (2) PAM is NNGRRT. The targeting domain for tier 4 gRNA molecules were selected based on (1) distance to a target site (e.g., start codon), e.g., within 500 bp (e.g., downstream) of the target site (e.g., start codon) and (2) PAM is NNGRRV. The targeting domain for tier 5 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon), (2) the presence of 5′G and (3) PAM is NNGRRT. The targeting domain for tier 6 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRT. The targeting domain for tier 7 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRV. Note that tiers are non-inclusive (each gRNA is listed only once for the strategy). In certain instances, no gRNA was identified based on the criteria of the particular tier.

Strategies to Identify gRNAs for S. pyogenes. S. Aureus, and N. meningitidis to Knock Out the UL54 Gene

As an example, three strategies were utilized to identify gRNAs for use with S. pyogenes, S. aureus and N. meningitidis Cas9 enzymes.

In the first strategy, guide RNAs (gRNAs) for use with the S. pyogenes (Tables 4A-4C) Cas9 were identified using the publically available web-based ZiFiT server (Fu et al., Improving CRISPR-Cas nuclease specificity using truncated guide RNAs. Nat Biotechnol. 2014 Jan. 26. doi: 10.1038/nbt.2808. PubMed PMID: 24463574, for the original references see Sander et al., 2007, NAR 35:W599-605; Sander et al., 2010, NAR 38: W462-8). In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available Repeat-Masker program. RepeatMmasker searches input DNA sequences for repeated elements and regions of low complexity. The output is a detailed annotation of the repeats present in a given query sequence. Following identification, gRNAs for use with a S. pyogenes Cas9 were ranked into 3 tiers.

The gRNAs in tier 1 were selected based on their distance to the target site and their orthogonality in the genome (based on the ZiFiT identification of close matches in the human genome containing an NGG PAM). As an example, for all targets, both 17-mer and 20-mer gRNAs were designed. gRNAs were also selected both for single-gRNA nuclease cutting and for the dual gRNA nickase strategy. Criteria for selecting gRNAs and the determination for which gRNAs can be used for which strategy is based on several considerations:
1. For the dual nickase strategy, gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, it will also often result in indel mutations at the site of only one of the gRNAs. Candidate pair members can be tested for how efficiently they remove the entire sequence versus just causing indel mutations at the site of one gRNA.

While it can be desirable to have gRNAs start with a 5′ G, this requirement was relaxed for some gRNAs in tier 1 in order to identify guides in the correct orientation, within a reasonable distance to the target position (i.e., within the first 500 bp of the coding sequence) and with a high level of orthogonality against the human genome. In order to find a pair for the dual-nickase strategy it was necessary to either extend the distance from the target position or remove the requirement for the 5′G. Tier 2 gRNAs were selected based on location within the first 500 bp of the coding sequence in the HSV gene. Tier 3 gRNAs were selected based on their location in the coding sequence, but downstream of the first 500 bp of the HSV gene. Note that tiers are non-inclusive (each gRNA is listed only once). In certain instances, no gRNA was identified based on the criteria of the particular tier.

As discussed above, gRNAs were identified for single-gRNA nuclease cleavage as well as for a dual-gRNA paired “nickase” strategy, as indicated.

gRNAs for use with the N. meningitidis (Tables 4F) and S. aureus (Tables 4D-4E) Cas9s were identified manually by scanning genomic DNA sequence for the presence of PAM sequences. These gRNAs were separated into two tiers for each species. The first tier includes gRNAs selected based on location in the first 500 bp of the coding sequence of the HSV gene. The second tier includes gRNAs selected based on location in the coding sequence, but downstream of the first 500 bp of the HSV gene.

In a second strategy, Guide RNAs (gRNAs) for use with S. pyogenes, S. aureus and N. meningitidis Cas9s were identified using a DNA sequence searching algorithm. Guide RNA design was carried out using a custom guide RNA design software based on the public tool cas-offinder (reference:Cas-OFFinder: a fast and versatile algorithm that searches for potential off-target sites of Cas9 RNA-guided endonucleases., Bioinformatics. 2014 Feb. 17. Bae S, Park J, Kim J S. PMID: 24463181). Said custom guide RNA design software scores guides after calculating their genome wide off-target propensity. Typically matches ranging from perfect matches to 7 mismatches are considered for guides ranging in length from 17 to 24. Once the off-target sites are computationally determined, an aggregate score is calculated for each guide and summarized in a tabular output using a web-interface. In addition to identifying potential gRNA sites adjacent to PAM sequences, the software also identifies all PAM adjacent sequences that differ by 1, 2, 3 or more nucleotides from the selected gRNA sites. Genomic DNA sequence for each gene was obtained from the UCSC Genome browser and sequences were screened for repeat elements using the publically available RepeatMasker program. RepeatMasker searches input DNA sequences for repeated elements and regions of low complexity. The output is a detailed annotation of the repeats present in a given query sequence.

Following identification, gRNAs were ranked into tiers based on their distance to the target site, their orthogonality and presence of a 5′ G (based on identification of close matches in the human genome containing a relavant PAM (e.g., in the case of S. pyogenes, a NGG PAM, in the case of S. aureus, a NNGRRT or NNGRRV PAM, and in the case of N. meningitidis, a NNNNGATT or NNNNGCTT PAM). Orthogonality refers to the number of sequences in the human genome that contain a minimum number of mismatches to the target sequence. A “high level of orthogonality” or “good orthogonality” may, for example, refer to 20-mer gRNAs that have no identical sequences in the human genome besides the intended target, nor any sequences that contain one or two mismatches in the target sequence. Targeting domains with good orthogonality are selected to minimize off-target DNA cleavage.

As an example, for S. pyogenes and N. meningitidis targets, 17-mer, or 20-mer gRNAs were designed. As another example, for S. aureus targets, 18-mer, 19-mer, 20-mer, 21-mer, 22-mer, 23-mer and 24-mer gRNAs were designed. Targeting domains, disclosed herein, may comprise the 17-mer described in Tables 14A-14E, 15A-15G or 16A-16C, e.g., the targeting domains of 18 or more nucleotides may comprise the 17-mer gRNAs described in Tables 14A-14E, 15A-15G or 16A-16C. Targeting domains, disclosed herein, may comprises the 18-mer described in Tables 14A-14E, 15A-15G or 16A-16C, e.g., the targeting domains of 19 or more nucleotides may comprise the 18-mer gRNAs described in Tables 14A-14E, 15A-15G or 16A-16C. Targeting domains, disclosed herein, may comprises the 19-mer described in Tables 14A-14E, 15A-15G or 16A-16C, e.g., the targeting domains of 20 or more nucleotides may comprise the 19-mer gRNAs described in Tables 14A-14E, 15A-15G or 16A-16C. Targeting domains, disclosed herein, may comprises the 20-mer gRNAs described in Tables 14A-14E, 15A-15G or 16A-16C, e.g., the targeting domains of 21 or more nucleotides may comprise the 20-mer gRNAs described in Tables 14A-14E, 15A-15G or 16A-16C. Targeting domains, disclosed herein, may comprises the 21-mer described in Tables 14A-14E, 15A-15G or 16A-16C, e.g., the targeting domains of 22 or more nucleotides may comprise the 21-mer gRNAs described in Tables 14A-14E, 15A-15G or 16A-16C. Targeting domains, disclosed herein, may comprises the 22-mer described in Tables 14A-14E, 15A-15G or 16A-16C, e.g., the targeting domains of 23 or more nucleotides may comprise the 22-mer gRNAs described in Tables 14A-14E, 15A-15G or 16A-16C. Targeting domains, disclosed herein, may comprises the 23-mer described in Tables 14A-14E, 15A-15G or 16A-16C, e.g., the targeting domains of 24 or more nucleotides may comprise the 23-mer gRNAs described in Tables 14A-14E, 15A-15G or 16A-16C. Targeting domains, disclosed herein, may comprises the 24-mer described in Tables 14A-14E, 15A-15G or 16A-16C, e.g., the targeting domains of 25 or more nucleotides may comprise the 24-mer gRNAs described in Tables 14A-14E, 15A-15G or 16A-16C. gRNAs were identified for both single-gRNA nuclease cleavage and for a dual-gRNA paired “nickase” strategy. Criteria for selecting gRNAs and the determination for which gRNAs can be used for the dual-gRNA paired “nickase” strategy is based on two considerations:
1. gRNA pairs should be oriented on the DNA such that PAMs are facing out and cutting with the D10A Cas9 nickase will result in 5′ overhangs.
2. An assumption that cleaving with dual nickase pairs will result in deletion of the entire intervening sequence at a reasonable frequency. However, cleaving with dual nickase pairs can also result in indel mutations at the site of only one of the gRNAs. Candidate pair members can be tested for how efficiently they remove the entire sequence versus causing indel mutations at the site of one gRNA.

The targeting domains discussed herein can be incorporated into the gRNAs described herein.

gRNAs were identified and ranked into 5 tiers for S. pyogenes (Tables 14A-14E), and N. meningitidis (Tables 16A-16C); and 7 tiers for S. aureus (Tables 15A-15G). For S. pyogenes, and N. meningitidis, the targeting domain for tier 1 gRNA molecules were selected based on (1) distance to a target site (e.g., start codon), e.g., within 500 bp (e.g., downstream) of the target site (e.g., start codon), (2) a high level of orthogonality and (3) the presence of 5′G. The targeting domain for tier 2 gRNA molecules were selected based on (1) distance to a target site (e.g., start codon), e.g., within 500 bp (e.g., downstream) of the target site (e.g., start codon) and (2) a high level of orthogonality. The targeting domain for tier 3 gRNA molecules were selected based on (1) distance to a target site (e.g., start codon), e.g., within 500 bp (e.g., downstream) of the target site (e.g., start codon) and (2) the presence of 5′G. The targeting domain for tier 4 gRNA molecules were selected based on distance to a target site (e.g., start codon), e.g., within 500 bp (e.g., downstream) of the target site (e.g., start codon). The targeting domain for tier 5 gRNA molecules were selected based on distance to the target site (e.g., start codon), e.g., within reminder of the coding sequence, e.g., downstream of the first 500 bp of coding sequence (e.g., anywhere from +500 (relative to the start codon) to the stop codon). For S. aureus, the targeting domain for tier 1 gRNA molecules were selected based on (1) distance to a target site (e.g., start codon), e.g., within 500 bp (e.g., downstream) of the target site (e.g., start codon), (2) a high level of orthogonality, (3) the presence of 5′G and (4) PAM is NNGRRT. The targeting domain for tier 2 gRNA molecules were selected based on (1) distance to a target site (e.g., start codon), e.g., within 500 bp (e.g., downstream) of the target site (e.g., start codon), (2) a high level of orthogonality, and (3) PAM is NNGRRT. The targeting domain for tier 3 gRNA molecules were selected based on (1) distance to a target site (e.g., start codon), e.g., within 500 bp (e.g., downstream) of the target site (e.g., start codon) and (2) PAM is NNGRRT. The targeting domain for tier 4 gRNA molecules were selected based on (1) distance to a target site (e.g., start codon), e.g., within 500 bp (e.g., downstream) of the target site (e.g., start codon) and (2) PAM is NNGRRV. The targeting domain for tier 5 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon), (2) the presence of 5′G and (3) PAM is NNGRRT. The targeting domain for tier 6 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRT. The targeting domain for tier 7 gRNA molecules were selected based on (1) distance to the target site (e.g., start codon), e.g., within reminder of the coding sequence, e.g., downstream of the first 500 bp of coding sequence (e.g., anywhere from +500 (relative to the start codon) to the stop codon) and (2) PAM is NNGRRV. Note that tiers are non-inclusive (each gRNA is listed only once for the strategy). In certain instances, no gRNA was identified based on the criteria of the particular tier.

In an embodiment, two or more (e.g., three or four) gRNA molecules are used with one Cas9 molecule. In another embodiment, when two or more (e.g., three or four) gRNAs are used with two or more Cas9 molecules, at least one Cas9 molecule is from a different species than the other Cas9 molecule(s). For example, when two gRNA molecules are used with two Cas9 molecules, one Cas9 molecule can be from one species and the other Cas9 molecule can be from a different species. Both Cas9 species are used to generate a single or double-strand break, as desired.

Any of the targeting domains in the tables described herein can be used with a Cas9 nickase molecule to generate a single strand break.

Any of the targeting domains in the tables described herein can be used with a Cas9 nuclease molecule to generate a double strand break.

When two gRNAs designed for use to target two Cas9 molecules, one Cas9 can be one species, the second Cas9 can be from a different species. Both Cas9 species are used to generate a single or double-strand break, as desired.

It is contemplated herein that any upstream gRNA described herein may be paired with any downstream gRNA described herein. When an upstream gRNA designed for use with one species of Cas9 is paired with a downstream gRNA designed for use from a different species of Cas9, both Cas9 species are used to generate a single or double-strand break, as desired.

### Exemplary Targeting Domains

Table 1A provides exemplary targeting domains for knocking out the UL19 gene selected according to first tier parameters. The targeting domains are selected based on location within the first 500 bp of the coding sequence of the UL19 gene and orthogonality against the human genome. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2A

| 1st Tier | DNA | | Target Site | |
|---|---|---|---|---|
| gRNA Name | Strand | Targeting Domain | Length | Seq ID |
| HSV1-UL19-3 | − | ACCGCGACCCUCCGGGAUAC | 20 | 387 |
| HSV1-UL19-4 | − | UCCGGGAUACCGGUAUGCCG | 20 | 388 |
| HSV1-UL19-5 | − | AUACCGGUAUGCCGCGGCCA | 20 | 389 |
| HSV1-UL19-97 | − | GCGACCCUCCGGGAUAC | 17 | 390 |
| HSV1-UL19-98 | − | GGGAUACCGGUAUGCCG | 17 | 391 |
| HSV1-UL19-99 | − | CCGGUAUGCCGCGGCCA | 17 | 392 |
| HSV1-UL19-11 | − | GUCGAGUUCGACGCCCUGCU | 20 | 393 |
| HSV1-UL19-12 | − | UCGAGUUCGACGCCCUGCUG | 20 | 394 |
| HSV1-UL19-13 | − | CCUGUCGCUCGUGCGCUUUC | 20 | 395 |
| HSV1-UL19-14 | − | CUCGUGCGCUUUCUGGAGCU | 20 | 396 |
| HSV1-UL19-15 | − | UCGUGCGCUUUCUGGAGCUC | 20 | 397 |
| HSV1-UL19-105 | − | GAGUUCGACGCCCUGCU | 17 | 398 |
| HSV1-UL19-106 | − | AGUUCGACGCCCUGCUG | 17 | 399 |
| HSV1-UL19-107 | − | GUCGCUCGUGCGCUUUC | 17 | 400 |
| HSV1-UL19-108 | − | GUGCGCUUUCUGGAGCU | 17 | 401 |
| HSV1-UL19-109 | − | UGCGCUUUCUGGAGCUC | 17 | 402 |
| HSV1-UL19-17 | − | UCUGGAGCUCGGGUUGUCGG | 20 | 403 |
| HSV1-UL19-18 | − | GUGCGUGUGUACCAAGUUUC | 20 | 404 |
| HSV1-UL19-19 | − | GUGUACCAAGUUUCCGGAGC | 20 | 405 |
| HSV1-UL19-111 | − | GGAGCUCGGGUUGUCGG | 17 | 406 |
| HSV1-UL19-112 | − | CGUGUGUACCAAGUUUC | 17 | 407 |
| HSV1-UL19-113 | − | UACCAAGUUUCCGGAGC | 17 | 408 |
| HSV1-UL19-23 | − | CGAGGGGCGCGUACAGUUCG | 20 | 409 |
| HSV1-UL19-24 | − | CAGCCGCUGAUUGCCCGCGA | 20 | 410 |
| HSV1-UL19-25 | − | UGACCAAGAUUAUCGACCGC | 20 | 411 |
| HSV1-UL19-26 | − | GACCAAGAUUAUCGACCGCC | 20 | 412 |
| HSV1-UL19-117 | − | GGGGCGCGUACAGUUCG | 17 | 413 |
| HSV1-UL19-118 | − | CCGCUGAUUGCCCGCGA | 17 | 414 |
| HSV1-UL19-119 | − | CCAAGAUUAUCGACCGC | 17 | 415 |
| HSV1-UL19-120 | − | CAAGAUUAUCGACCGCC | 17 | 416 |
| HSV1-UL19-31 | − | AGGCCAUCGCCCUGCUCACG | 20 | 417 |
| HSV1-UL19-125 | − | CCAUCGCCCUGCUCACG | 17 | 418 |
| HSV1-UL19-33 | − | CAUCGCCCUGCUCACGGGGG | 20 | 419 |
| HSV1-UL19-127 | − | CGCCCUGCUCACGGGGG | 17 | 420 |
| HSV1-UL19-40 | + | GCUGUUGGAUGGCGCGCAAC | 20 | 421 |
| HSV1-UL19-134 | + | GUUGGAUGGCGCGCAAC | 17 | 422 |
| HSV1-UL19-41 | + | CGCCGAUACCCGUCCCGUCC | 20 | 423 |
| HSV1-UL19-135 | + | CGAUACCCGUCCCGUCC | 17 | 424 |
| HSV1-UL19-58 | + | AUUGUGUGUGGGUUGCUCGA | 20 | 425 |
| HSV1-UL19-59 | + | UUGUGUGUGGGUUGCUCGAU | 20 | 426 |
| HSV1-UL19-152 | + | GUGUGUGGGUUGCUCGA | 17 | 427 |
| HSV1-UL19-153 | + | UGUGUGGGUUGCUCGAU | 17 | 428 |
| HSV1-UL19-64 | + | GAUGGGGUGCGGGCCGUCGC | 20 | 429 |
| HSV1-UL19-65 | + | GGGCCGUCGCGGGCAAUCAG | 20 | 430 |
| HSV1-UL19-158 | + | GGGGUGCGGGCCGUCGC | 17 | 431 |
| HSV1-UL19-159 | + | CCGUCGCGGGCAAUCAG | 17 | 432 |
| HSV1-UL19-67 | + | CGCGGGCAAUCAGCGGCUGG | 20 | 433 |
| HSV1-UL19-161 | + | GGGCAAUCAGCGGCUGG | 17 | 434 |
| HSV1-UL19-69 | + | UCGUUCAUGUAGGCCAGCUC | 20 | 435 |
| HSV1-UL19-70 | + | GUAGGCCAGCUCCGGAAACU | 20 | 436 |
| HSV1-UL19-71 | + | CCAGAAAGCGCACGAGCGAC | 20 | 437 |
| HSV1-UL19-72 | + | CAGAAAGCGCACGAGCGACA | 20 | 438 |
| HSV1-UL19-73 | + | UGUUGCAAUACGACCCCAGC | 20 | 439 |
| HSV1-UL19-74 | + | GUUGCAAUACGACCCCAGCA | 20 | 440 |
| HSV1-UL19-75 | + | CGUCGAACUCGACGUCGUAC | 20 | 441 |
| HSV1-UL19-76 | + | GUCGUACAGGCUGUUUGCAU | 20 | 442 |
| HSV1-UL19-163 | + | UUCAUGUAGGCCAGCUC | 17 | 443 |
| HSV1-UL19-164 | + | GGCCAGCUCCGGAAACU | 17 | 444 |
| HSV1-UL19-165 | + | GAAAGCGCACGAGCGAC | 17 | 445 |
| HSV1-UL19-166 | + | AAAGCGCACGAGCGACA | 17 | 446 |
| HSV1-UL19-167 | + | UGCAAUACGACCCCAGC | 17 | 447 |
| HSV1-UL19-168 | + | GCAAUACGACCCCAGCA | 17 | 448 |
| HSV1-UL19-169 | + | CGAACUCGACGUCGUAC | 17 | 449 |
| HSV1-UL19-170 | + | GUACAGGCUGUUUGCAU | 17 | 450 |
| HSV1-UL19-82 | + | CACCUCGAUCGUGCUAAGGA | 20 | 451 |
| HSV1-UL19-176 | + | CUCGAUCGUGCUAAGGA | 17 | 452 |
| HSV1-UL19-87 | + | GCACCAUGGCCGCGGCAUAC | 20 | 453 |
| HSV1-UL19-181 | + | CCAUGGCCGCGGCAUAC | 17 | 454 |
| HSV1-UL19-91 | + | ACCGGUAUCCCGGAGGGUCG | 20 | 455 |
| HSV1-UL19-92 | + | GUAUCCCGGAGGGUCGCGGU | 20 | 456 |
| HSV1-UL19-93 | + | UAUCCCGGAGGGUCGCGGUU | 20 | 457 |
| HSV1-UL19-185 | + | GGUAUCCCGGAGGGUCG | 17 | 458 |
| HSV1-UL19-186 | + | UCCCGGAGGGUCGCGGU | 17 | 459 |
| HSV1-UL19-187 | + | CCCGGAGGGUCGCGGUU | 17 | 460 |

Table 1B provides exemplary targeting domains for knocking out the UL19 gene selected according to the second tier parameters. The targeting domains are selected based on location within the first 500 bp of the coding sequence of the UL19 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 1B

| 2nd Tier | DNA | | Target Site | |
|---|---|---|---|---|
| gRNA Name | Strand | Targeting Domain | Length | Seq ID |
| HSV1-UL19-1 | − | CGCUCCCAACCGCGACCCUC | 20 | 461 |
| HSV1-UL19-2 | − | GCUCCCAACCGCGACCCUCC | 20 | 462 |
| HSV1-UL19-6 | − | GCCGCGGCCAUGGUGCCGAC | 20 | 463 |
| HSV1-UL19-7 | − | CCGCGGCCAUGGUGCCGACC | 20 | 464 |
| HSV1-UL19-8 | − | GUCCCUCCUUAGCACGAUCG | 20 | 465 |
| HSV1-UL19-9 | − | CCUCCUUAGCACGAUCGAGG | 20 | 466 |
| HSV1-UL19-10 | − | CGUCGAGUUCGACGCCCUGC | 20 | 467 |
| HSV1-UL19-16 | − | CUUUCUGGAGCUCGGGUUGU | 20 | 468 |
| HSV1-UL19-20 | − | GGAGCUGGCCUACAUGAACG | 20 | 469 |
| HSV1-UL19-21 | − | GAGCUGGCCUACAUGAACGA | 20 | 470 |
| HSV1-UL19-22 | − | AGCUGGCCUACAUGAACGAG | 20 | 471 |
| HSV1-UL19-27 | − | CCUGAACGCCGCCUUCAGCC | 20 | 472 |
| HSV1-UL19-28 | − | CGCCUUCAGCCUGGCCACCG | 20 | 473 |
| HSV1-UL19-29 | − | CGAGGCCAUCGCCCUGCUCA | 20 | 474 |
| HSV1-UL19-30 | − | GAGGCCAUCGCCCUGCUCAC | 20 | 475 |
| HSV1-UL19-32 | − | GGCCAUCGCCCUGCUCACGG | 20 | 476 |
| HSV1-UL19-34 | − | CCUGCUCACGGGGGAGGCCC | 20 | 477 |
| HSV1-UL19-35 | − | CUCACGGGGGAGGCCCUGGA | 20 | 478 |
| HSV1-UL19-36 | − | UCACGGGGGAGGCCCUGGAC | 20 | 479 |
| HSV1-UL19-37 | − | GGGGGAGGCCCUGGACGGGA | 20 | 480 |
| HSV1-UL19-38 | − | GGGGAGGCCCUGGACGGGAC | 20 | 481 |
| HSV1-UL19-39 | − | GCCCUGGACGGGACGGGUAU | 20 | 482 |
| HSV1-UL19-42 | + | GCCGAUACCCGUCCCGUCCA | 20 | 483 |
| HSV1-UL19-43 | + | CCAGGGCCUCCCCCGUGAGC | 20 | 484 |
| HSV1-UL19-44 | + | CAGGGCCUCCCCCGUGAGCA | 20 | 485 |
| HSV1-UL19-45 | + | CUCCCCCGUGAGCAGGGCGA | 20 | 486 |
| HSV1-UL19-46 | + | CGUGAGCAGGGCGAUGGCCU | 20 | 487 |
| HSV1-UL19-47 | + | GAGCAGGGCGAUGGCCUCGG | 20 | 488 |
| HSV1-UL19-48 | + | GGGCGAUGGCCUCGGUGGCC | 20 | 489 |
| HSV1-UL19-49 | + | GGCCUCGGUGGCCAGGCUGA | 20 | 490 |
| HSV1-UL19-50 | + | CUCGGUGGCCAGGCUGAAGG | 20 | 491 |
| HSV1-UL19-51 | + | CCAGGCUGAAGGCGGCGUUC | 20 | 492 |
| HSV1-UL19-52 | + | CAGGCUGAAGGCGGCGUUCA | 20 | 493 |
| HSV1-UL19-53 | + | UGAAGGCGGCGUUCAGGGCC | 20 | 494 |
| HSV1-UL19-54 | + | AGGCGGCGUUCAGGGCCCGG | 20 | 495 |
| HSV1-UL19-55 | + | GGCCCGGCGGUCGAUAAUCU | 20 | 496 |
| HSV1-UL19-56 | + | CUUGGUCAUGUAAUUGUGUG | 20 | 497 |
| HSV1-UL19-57 | + | UUGGUCAUGUAAUUGUGUGU | 20 | 498 |
| HSV1-UL19-60 | + | UGUGUGUGGGUUGCUCGAUG | 20 | 499 |
| HSV1-UL19-61 | + | GUGGGUUGCUCGAUGGGGUG | 20 | 500 |
| HSV1-UL19-62 | + | UGGGUUGCUCGAUGGGGUGC | 20 | 501 |
| HSV1-UL19-63 | + | CGAUGGGGUGCGGGCCGUCG | 20 | 502 |
| HSV1-UL19-66 | + | CGUCGCGGGCAAUCAGCGGC | 20 | 503 |
| HSV1-UL19-68 | + | UACGCGCCCCUCGUUCAUGU | 20 | 504 |
| HSV1-UL19-77 | + | UGUUUGCAUCGGAGCGCACG | 20 | 505 |
| HSV1-UL19-78 | + | GUUUGCAUCGGAGCGCACGC | 20 | 506 |
| HSV1-UL19-79 | + | CGCGGGAAAAAAAAUCAAAC | 20 | 507 |
| HSV1-UL19-80 | + | ACGCCACCUCGAUCGUGCUA | 20 | 508 |
| HSV1-UL19-81 | + | CCACCUCGAUCGUGCUAAGG | 20 | 509 |
| HSV1-UL19-83 | + | GAUCGUGCUAAGGAGGGACC | 20 | 510 |
| HSV1-UL19-84 | + | GUGCUAAGGAGGGACCCGGU | 20 | 511 |
| HSV1-UL19-85 | + | GAGGGACCCGGUCGGCACCA | 20 | 512 |
| HSV1-UL19-86 | + | CCCGGUCGGCACCAUGGCCG | 20 | 513 |
| HSV1-UL19-88 | + | GCCGCGGCAUACCGGUAUCC | 20 | 514 |
| HSV1-UL19-89 | + | GCGGCAUACCGGUAUCCCGG | 20 | 515 |
| HSV1-UL19-90 | + | CGGCAUACCGGUAUCCCGGA | 20 | 516 |
| HSV1-UL19-94 | + | CGGAGGGUCGCGGUUGGGAG | 20 | 517 |
| HSV1-UL19-95 | − | UCCCAACCGCGACCCUC | 17 | 518 |
| HSV1-UL19-96 | − | CCCAACCGCGACCCUCC | 17 | 519 |
| HSV1-UL19-100 | − | GCGGCCAUGGUGCCGAC | 17 | 520 |
| HSV1-UL19-101 | − | CGGCCAUGGUGCCGACC | 17 | 521 |
| HSV1-UL19-102 | − | CCUCCUUAGCACGAUCG | 17 | 522 |
| HSV1-UL19-103 | − | CCUUAGCACGAUCGAGG | 17 | 523 |
| HSV1-UL19-104 | − | CGAGUUCGACGCCCUGC | 17 | 524 |
| HSV1-UL19-110 | − | UCUGGAGCUCGGGUUGU | 17 | 525 |
| HSV1-UL19-114 | − | GCUGGCCUACAUGAACG | 17 | 526 |
| HSV1-UL19-115 | − | CUGGCCUACAUGAACGA | 17 | 527 |
| HSV1-UL19-116 | − | UGGCCUACAUGAACGAG | 17 | 528 |
| HSV1-UL19-121 | − | GAACGCCGCCUUCAGCC | 17 | 529 |
| HSV1-UL19-122 | − | CUUCAGCCUGGCCACCG | 17 | 530 |
| HSV1-UL19-123 | − | GGCCAUCGCCCUGCUCA | 17 | 531 |
| HSV1-UL19-124 | − | GCCAUCGCCCUGCUCAC | 17 | 532 |
| HSV1-UL19-126 | − | CAUCGCCCUGCUCACGG | 17 | 533 |
| HSV1-UL19-128 | − | GCUCACGGGGGAGGCCC | 17 | 534 |
| HSV1-UL19-129 | − | ACGGGGGAGGCCCUGGA | 17 | 535 |
| HSV1-UL19-130 | − | CGGGGGAGGCCCUGGAC | 17 | 536 |
| HSV1-UL19-131 | − | GGAGGCCCUGGACGGGA | 17 | 537 |
| HSV1-UL19-132 | − | GAGGCCCUGGACGGGAC | 17 | 538 |
| HSV1-UL19-133 | − | CUGGACGGGACGGGUAU | 17 | 539 |
| HSV1-UL19-136 | + | GAUACCCGUCCCGUCCA | 17 | 540 |
| HSV1-UL19-137 | + | GGGCCUCCCCCGUGAGC | 17 | 541 |
| HSV1-UL19-138 | + | GGCCUCCCCCGUGAGCA | 17 | 542 |
| HSV1-UL19-139 | + | CCCCGUGAGCAGGGCGA | 17 | 543 |
| HSV1-UL19-140 | + | GAGCAGGGCGAUGGCCU | 17 | 544 |
| HSV1-UL19-141 | + | CAGGGCGAUGGCCUCGG | 17 | 545 |
| HSV1-UL19-142 | + | CGAUGGCCUCGGUGGCC | 17 | 546 |
| HSV1-UL19-143 | + | CUCGGUGGCCAGGCUGA | 17 | 547 |
| HSV1-UL19-144 | + | GGUGGCCAGGCUGAAGG | 17 | 548 |
| HSV1-UL19-145 | + | GGCUGAAGGCGGCGUUC | 17 | 549 |
| HSV1-UL19-146 | + | GCUGAAGGCGGCGUUCA | 17 | 550 |
| HSV1-UL19-147 | + | AGGCGGCGUUCAGGGCC | 17 | 551 |
| HSV1-UL19-148 | + | CGGCGUUCAGGGCCCGG | 17 | 552 |
| HSV1-UL19-149 | + | CCGGCGGUCGAUAAUCU | 17 | 553 |
| HSV1-UL19-150 | + | GGUCAUGUAAUUGUGUG | 17 | 554 |
| HSV1-UL19-151 | + | GUCAUGUAAUUGUGUGU | 17 | 555 |
| HSV1-UL19-154 | + | GUGUGGGUUGCUCGAUG | 17 | 556 |
| HSV1-UL19-155 | + | GGUUGCUCGAUGGGGUG | 17 | 557 |
| HSV1-UL19-156 | + | GUUGCUCGAUGGGGUGC | 17 | 558 |
| HSV1-UL19-157 | + | UGGGGUGCGGGCCGUCG | 17 | 559 |
| HSV1-UL19-160 | + | CGCGGGCAAUCAGCGGC | 17 | 560 |
| HSV1-UL19-162 | + | GCGCCCCUCGUUCAUGU | 17 | 561 |
| HSV1-UL19-171 | + | UUGCAUCGGAGCGCACG | 17 | 562 |
| HSV1-UL19-172 | + | UGCAUCGGAGCGCACGC | 17 | 563 |
| HSV1-UL19-173 | + | GGGAAAAAAAAUCAAAC | 17 | 564 |
| HSV1-UL19-174 | + | CCACCUCGAUCGUGCUA | 17 | 565 |
| HSV1-UL19-175 | + | CCUCGAUCGUGCUAAGG | 17 | 566 |
| HSV1-UL19-177 | + | CGUGCUAAGGAGGGACC | 17 | 567 |
| HSV1-UL19-178 | + | CUAAGGAGGGACCCGGU | 17 | 568 |
| HSV1-UL19-179 | + | GGACCCGGUCGGCACCA | 17 | 569 |
| HSV1-UL19-180 | + | GGUCGGCACCAUGGCCG | 17 | 570 |
| HSV1-UL19-182 | + | GCGGCAUACCGGUAUCC | 17 | 571 |
| HSV1-UL19-183 | + | GCAUACCGGUAUCCCGG | 17 | 572 |
| HSV1-UL19-184 | + | CAUACCGGUAUCCCGGA | 17 | 573 |
| HSV1-UL19-188 | + | AGGGUCGCGGUUGGGAG | 17 | 574 |

Table 1C provides exemplary targeting domains for knocking out the UL19 gene selected according to the third tier parameters. The targeting domains are selected based on location within the coding sequence, but downstream of the first 500 bp of the UL19 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 1C

| 3rd Tier | DNA | | Target Site | |
|---|---|---|---|---|
| gRNA Name | Strand | Targeting Domain | Length | Seq ID |
| HSV1-UL19-189 | − | CGCAACGUCCAGGCCGUCCU | 20 | 575 |
| HSV1-UL19-190 | − | GCAACGUCCAGGCCGUCCUC | 20 | 576 |
| HSV1-UL19-191 | − | CAACGUCCAGGCCGUCCUCG | 20 | 577 |
| HSV1-UL19-192 | − | GUCCUCGGGGCGUUUGAGCG | 20 | 578 |
| HSV1-UL19-193 | − | CGGGGCGUUUGAGCGCGGCA | 20 | 579 |
| HSV1-UL19-194 | − | CCAGAUGCUGCACGUGCUGC | 20 | 580 |
| HSV1-UL19-195 | − | GCUGCACGUGCUGCUGGAAA | 20 | 581 |
| HSV1-UL19-196 | − | GCUGGAAAAGGCGCCGCCCC | 20 | 582 |
| HSV1-UL19-197 | − | GUUGCCGAUGCAACGAUACC | 20 | 583 |
| HSV1-UL19-198 | − | AUGCAACGAUACCUGGACAA | 20 | 584 |
| HSV1-UL19-199 | − | AUACCUGGACAACGGCCGCC | 20 | 585 |
| HSV1-UL19-200 | − | ACAACGGCCGCCUGGCCACC | 20 | 586 |
| HSV1-UL19-201 | − | CAACGGCCGCCUGGCCACCA | 20 | 587 |
| HSV1-UL19-202 | − | CGGCCGCCUGGCCACCAGGG | 20 | 588 |
| HSV1-UL19-203 | − | GCCUGGCCACCAGGGUGGCC | 20 | 589 |
| HSV1-UL19-204 | − | CCUGGCCACCAGGGUGGCCC | 20 | 590 |
| HSV1-UL19-205 | − | CAGGGUGGCCCGGGCGACCC | 20 | 591 |
| HSV1-UL19-206 | − | CUGCGAGACGAGCUUUUUCC | 20 | 592 |
| HSV1-UL19-207 | − | UGCGAGACGAGCUUUUUCCU | 20 | 593 |
| HSV1-UL19-208 | − | GACGAGCUUUUUCCUGGGCA | 20 | 594 |
| HSV1-UL19-209 | − | GAGCUUUUUCCUGGGCAAGG | 20 | 595 |
| HSV1-UL19-210 | − | AGCUUUUUCCUGGGCAAGGC | 20 | 596 |
| HSV1-UL19-211 | − | UGGGCAAGGCGGGCCACCGC | 20 | 597 |
| HSV1-UL19-212 | − | GGGCAAGGCGGGCCACCGCC | 20 | 598 |
| HSV1-UL19-213 | − | CAAGGCGGGCCACCGCCGGG | 20 | 599 |
| HSV1-UL19-214 | − | CCACCGCCGGGAGGCCGUCG | 20 | 600 |
| HSV1-UL19-215 | − | GCCGGGAGGCCGUCGAGGCC | 20 | 601 |
| HSV1-UL19-216 | − | GGCCGUCGAGGCCUGGCUCG | 20 | 602 |
| HSV1-UL19-217 | − | CUGGCUCGUGGACCUCACCA | 20 | 603 |
| HSV1-UL19-218 | − | CACGGCCACGCAGCCCUCCG | 20 | 604 |
| HSV1-UL19-219 | − | CUGACGCAUGCCGACACGCG | 20 | 605 |
| HSV1-UL19-220 | − | UGACGCAUGCCGACACGCGC | 20 | 606 |
| HSV1-UL19-221 | − | CGCAUGCCGACACGCGCGGG | 20 | 607 |
| HSV1-UL19-222 | − | UGCCGACACGCGCGGGCGGC | 20 | 608 |
| HSV1-UL19-223 | − | ACGCGCGGGCGGCCGGUCGA | 20 | 609 |
| HSV1-UL19-224 | − | CGCGCGGGCGGCCGGUCGAC | 20 | 610 |
| HSV1-UL19-225 | − | GCGCGGGCGGCCGGUCGACG | 20 | 611 |
| HSV1-UL19-226 | − | CCUGCAGUCCUUCCUGAAAG | 20 | 612 |
| HSV1-UL19-227 | − | GCAGUCCUUCCUGAAAGUGG | 20 | 613 |
| HSV1-UL19-228 | − | GGACACCGAAGCCGACGUGC | 20 | 614 |
| HSV1-UL19-229 | − | GCCGACGUGCCGGUGACGUA | 20 | 615 |
| HSV1-UL19-230 | − | GCCGGUGACGUACGGCGAGA | 20 | 616 |
| HSV1-UL19-231 | − | UACGGCGAGAUGGUCCUGAA | 20 | 617 |
| HSV1-UL19-232 | − | ACGGCGAGAUGGUCCUGAAC | 20 | 618 |
| HSV1-UL19-233 | − | CGGCGAGAUGGUCCUGAACG | 20 | 619 |
| HSV1-UL19-234 | − | GGUCCUGAACGGGGCCAACC | 20 | 620 |
| HSV1-UL19-235 | − | GAACGGGGCCAACCUGGUCA | 20 | 621 |
| HSV1-UL19-236 | − | CCUGGUCACGGCGCUCGUGA | 20 | 622 |
| HSV1-UL19-237 | − | CUGGUCACGGCGCUCGUGAU | 20 | 623 |
| HSV1-UL19-238 | − | CACGGCGCUCGUGAUGGGCA | 20 | 624 |
| HSV1-UL19-239 | − | GGGCAAGGCCGUGCGAAGUC | 20 | 625 |
| HSV1-UL19-240 | − | CGUGCGAAGUCUGGACGACG | 20 | 626 |
| HSV1-UL19-241 | − | GUGCGAAGUCUGGACGACGU | 20 | 627 |
| HSV1-UL19-242 | − | CGACGUGGGCCGCCACCUGC | 20 | 628 |
| HSV1-UL19-243 | − | CCGCCACCUGCUGGAGAUGC | 20 | 629 |
| HSV1-UL19-244 | − | CCACCUGCUGGAGAUGCAGG | 20 | 630 |
| HSV1-UL19-245 | − | AGGAGCAGCUCGACCUGAAC | 20 | 631 |
| HSV1-UL19-246 | − | CGACCUGAACCGGCAGACGC | 20 | 632 |
| HSV1-UL19-247 | − | CCAGACGACGCGCGUGCGCG | 20 | 633 |
| HSV1-UL19-248 | − | GACGCGCGUGCGCGCGGAUC | 20 | 634 |
| HSV1-UL19-249 | − | CGCGCGGAUCUGGUGUCCAU | 20 | 635 |
| HSV1-UL19-250 | − | GGUGUCCAUCGGCGAGAAGC | 20 | 636 |
| HSV1-UL19-251 | − | CGGCGAGAAGCUGGUCUUUC | 20 | 637 |
| HSV1-UL19-252 | − | CGAGAAGCUGGUCUUUCUGG | 20 | 638 |
| HSV1-UL19-253 | − | GCUGGUCUUUCUGGAGGCCC | 20 | 639 |
| HSV1-UL19-254 | − | CACCAACGUUCCCUACCCCC | 20 | 640 |
| HSV1-UL19-255 | − | CAACGUUCCCUACCCCCUGG | 20 | 641 |
| HSV1-UL19-256 | − | AACGUUCCCUACCCCCUGGU | 20 | 642 |
| HSV1-UL19-257 | − | CUACCCCCUGGUGGGCGCCA | 20 | 643 |
| HSV1-UL19-258 | − | CCUGACGUUCGUCCUGCCCC | 20 | 644 |
| HSV1-UL19-259 | − | CUGACGUUCGUCCUGCCCCU | 20 | 645 |
| HSV1-UL19-260 | − | GCCCCUGGGCCUGUUCAAUC | 20 | 646 |
| HSV1-UL19-261 | − | GGGCCUGUUCAAUCCGGUCA | 20 | 647 |
| HSV1-UL19-262 | − | UGUUCAAUCCGGUCAUGGAA | 20 | 648 |
| HSV1-UL19-263 | − | GAACGGUUUGCCGCGCACGC | 20 | 649 |
| HSV1-UL19-264 | − | AACGGUUUGCCGCGCACGCC | 20 | 650 |
| HSV1-UL19-265 | − | ACGGUUUGCCGCGCACGCCG | 20 | 651 |
| HSV1-UL19-266 | − | GGGGACCUAGUCCCCGCCCC | 20 | 652 |
| HSV1-UL19-267 | − | AGUCCCCGCCCCCGGCCACC | 20 | 653 |
| HSV1-UL19-268 | − | CGCCCCGCCAGCUGUUUUUU | 20 | 654 |
| HSV1-UL19-269 | − | GCCCCGCCAGCUGUUUUUUU | 20 | 655 |
| HSV1-UL19-270 | − | CCCCGCCAGCUGUUUUUUUG | 20 | 656 |
| HSV1-UL19-271 | − | CCCGCCAGCUGUUUUUUUGG | 20 | 657 |
| HSV1-UL19-272 | − | CCAGCUGUUUUUUUGGGGGA | 20 | 658 |
| HSV1-UL19-273 | − | UUUUUGGGGGAAGGACCGCC | 20 | 659 |
| HSV1-UL19-274 | − | CCAGGUGCUGCGCCUGUCUC | 20 | 660 |
| HSV1-UL19-275 | − | CUGUCUCUGGAACACGCGAU | 20 | 661 |
| HSV1-UL19-276 | − | UGUCUCUGGAACACGCGAUC | 20 | 662 |
| HSV1-UL19-277 | − | UUCGCUGAUGAACGUUGACG | 20 | 663 |
| HSV1-UL19-278 | − | GCUGAUGAACGUUGACGCGG | 20 | 664 |
| HSV1-UL19-279 | − | AUGAACGUUGACGCGGCGGU | 20 | 665 |
| HSV1-UL19-280 | − | UGAACGUUGACGCGGCGGUC | 20 | 666 |
| HSV1-UL19-281 | − | GAACGUUGACGCGGCGGUCG | 20 | 667 |
| HSV1-UL19-282 | − | AACGUUGACGCGGCGGUCGG | 20 | 668 |
| HSV1-UL19-283 | − | GUCGAAGCCGCCAAUCCGUA | 20 | 669 |
| HSV1-UL19-284 | − | UCGAAGCCGCCAAUCCGUAC | 20 | 670 |
| HSV1-UL19-285 | − | CGAAGCCGCCAAUCCGUACG | 20 | 671 |
| HSV1-UL19-286 | − | CAAUCCGUACGGGGCGUACG | 20 | 672 |
| HSV1-UL19-287 | − | UCCGUACGGGGCGUACGUGG | 20 | 673 |
| HSV1-UL19-288 | − | CGGGGCGUACGUGGCGGCCC | 20 | 674 |
| HSV1-UL19-289 | − | GCGUACGUGGCGGCCCCGGC | 20 | 675 |
| HSV1-UL19-290 | − | AGCAGCUGUUUUUGAACGCC | 20 | 676 |
| HSV1-UL19-291 | − | GCAGCUGUUUUUGAACGCCU | 20 | 677 |
| HSV1-UL19-292 | − | CAGCUGUUUUUGAACGCCUG | 20 | 678 |
| HSV1-UL19-293 | − | AGCUGUUUUUGAACGCCUGG | 20 | 679 |
| HSV1-UL19-294 | − | GAACGCCUGGGGGCAGCGCC | 20 | 680 |
| HSV1-UL19-295 | − | UGGGGGCAGCGCCUGGCCCA | 20 | 681 |
| HSV1-UL19-296 | − | GGGGGCAGCGCCUGGCCCAC | 20 | 682 |
| HSV1-UL19-297 | − | GGCAGCGCCUGGCCCACGGG | 20 | 683 |
| HSV1-UL19-298 | − | GCAGCGCCUGGCCCACGGGC | 20 | 684 |
| HSV1-UL19-299 | − | UGGCCCACGGGCGGGUCCGA | 20 | 685 |
| HSV1-UL19-300 | − | GGCCCACGGGCGGGUCCGAU | 20 | 686 |
| HSV1-UL19-301 | − | CGGGCGGGUCCGAUGGGUCG | 20 | 687 |
| HSV1-UL19-302 | − | CGGGUCCGAUGGGUCGCGGA | 20 | 688 |
| HSV1-UL19-303 | − | CGCGGAAGGCCAGAUGACCC | 20 | 689 |
| HSV1-UL19-304 | − | GCAGCCCGACAACGCCAACC | 20 | 690 |
| HSV1-UL19-305 | − | CCCCGCGUUCGACUUCUUUG | 20 | 691 |
| HSV1-UL19-306 | − | CCCGCGUUCGACUUCUUUGU | 20 | 692 |
| HSV1-UL19-307 | − | CCGCGUUCGACUUCUUUGUG | 20 | 693 |
| HSV1-UL19-308 | − | CGCGUUCGACUUCUUUGUGG | 20 | 694 |
| HSV1-UL19-309 | − | GUUCGACUUCUUUGUGGGGG | 20 | 695 |
| HSV1-UL19-310 | − | CUUUGUGGGGGUGGCCGACG | 20 | 696 |
| HSV1-UL19-311 | − | GGUGGCCGACGUGGAGCUGC | 20 | 697 |
| HSV1-UL19-312 | − | GUGGCCGACGUGGAGCUGCC | 20 | 698 |
| HSV1-UL19-313 | − | UGGCCGACGUGGAGCUGCCG | 20 | 699 |
| HSV1-UL19-314 | − | GGCCGACGUGGAGCUGCCGG | 20 | 700 |
| HSV1-UL19-315 | − | GCCGACGUGGAGCUGCCGGG | 20 | 701 |
| HSV1-UL19-316 | − | CCGACGUGGAGCUGCCGGGG | 20 | 702 |
| HSV1-UL19-317 | − | CGACGUGGAGCUGCCGGGGG | 20 | 703 |
| HSV1-UL19-318 | − | GCCGGGGGGGGACGUUCCCC | 20 | 704 |
| HSV1-UL19-319 | − | GGGGGGGACGUUCCCCCGGC | 20 | 705 |
| HSV1-UL19-320 | − | GGACGUUCCCCCGGCCGGCC | 20 | 706 |
| HSV1-UL19-321 | − | GACGUUCCCCCGGCCGGCCC | 20 | 707 |
| HSV1-UL19-322 | − | ACGUUCCCCCGGCCGGCCCG | 20 | 708 |
| HSV1-UL19-323 | − | CGUUCCCCCGGCCGGCCCGG | 20 | 709 |
| HSV1-UL19-324 | − | GGCCGGCCCGGGGGAGAUCC | 20 | 710 |
| HSV1-UL19-325 | − | CGGGGGAGAUCCAGGCCACC | 20 | 711 |
| HSV1-UL19-326 | − | GAUCCAGGCCACCUGGCGCG | 20 | 712 |
| HSV1-UL19-327 | − | GCCACCUGGCGCGUGGUGAA | 20 | 713 |
| HSV1-UL19-328 | − | GGUGAACGGCAACUUGCCCC | 20 | 714 |
| HSV1-UL19-329 | − | CUUGCCCCUGGCGCUAUGUC | 20 | 715 |
| HSV1-UL19-330 | − | GCCCCUGGCGCUAUGUCCGG | 20 | 716 |
| HSV1-UL19-331 | − | CGCUAUGUCCGGCGGCGUUC | 20 | 717 |
| HSV1-UL19-332 | − | GCUAUGUCCGGCGGCGUUCC | 20 | 718 |
| HSV1-UL19-333 | − | CGGCGGCGUUCCGGGACGCC | 20 | 719 |
| HSV1-UL19-334 | − | GGCGGCGUUCCGGGACGCCC | 20 | 720 |
| HSV1-UL19-335 | − | GCGGCGUUCCGGGACGCCCG | 20 | 721 |
| HSV1-UL19-336 | − | GUUCCGGGACGCCCGGGGCC | 20 | 722 |
| HSV1-UL19-337 | − | GGACGCCCGGGGCCUGGAGC | 20 | 723 |
| HSV1-UL19-338 | − | GACGCCCGGGGCCUGGAGCU | 20 | 724 |
| HSV1-UL19-339 | − | ACGCCCGGGGCCUGGAGCUG | 20 | 725 |
| HSV1-UL19-340 | − | CGCCCGGGGCCUGGAGCUGG | 20 | 726 |
| HSV1-UL19-341 | − | CCGGGGCCUGGAGCUGGGGG | 20 | 727 |
| HSV1-UL19-342 | − | CGGGGCCUGGAGCUGGGGGU | 20 | 728 |
| HSV1-UL19-343 | − | GGGGGUGGGACGCCACGCCA | 20 | 729 |
| HSV1-UL19-344 | − | GCCACCAUCGCCGCCGUUCG | 20 | 730 |
| HSV1-UL19-345 | − | CCACCAUCGCCGCCGUUCGC | 20 | 731 |
| HSV1-UL19-346 | − | CACCAUCGCCGCCGUUCGCG | 20 | 732 |
| HSV1-UL19-347 | − | GUUCGACGACCGCAACUACC | 20 | 733 |
| HSV1-UL19-348 | − | CGACGACCGCAACUACCCGG | 20 | 734 |
| HSV1-UL19-349 | − | GGCGGUGUUUUACCUGCUGC | 20 | 735 |
| HSV1-UL19-350 | − | CUGCUGCAGGCCGCCAUACA | 20 | 736 |
| HSV1-UL19-351 | − | CGAGCACGUCUUCUGCGCCC | 20 | 737 |
| HSV1-UL19-352 | − | ACGUCUUCUGCGCCCUGGCC | 20 | 738 |
| HSV1-UL19-353 | − | CUGCGCCCUGGCCCGGCUCG | 20 | 739 |
| HSV1-UL19-354 | − | UCCAGUGCAUCACCAGCUAC | 20 | 740 |
| HSV1-UL19-355 | − | CCAGCUACUGGAACAACACG | 20 | 741 |
| HSV1-UL19-356 | − | CUGGAACAACACGCGGUGCG | 20 | 742 |
| HSV1-UL19-357 | − | GUUCGUGAACGACUACUCGC | 20 | 743 |
| HSV1-UL19-358 | − | UCGUACGUCGUGACCUACCU | 20 | 744 |
| HSV1-UL19-359 | − | CGUACGUCGUGACCUACCUC | 20 | 745 |
| HSV1-UL19-360 | − | GUACGUCGUGACCUACCUCG | 20 | 746 |
| HSV1-UL19-361 | − | UACGUCGUGACCUACCUCGG | 20 | 747 |
| HSV1-UL19-362 | − | CCUCGGGGGAGACCUUCCCG | 20 | 748 |
| HSV1-UL19-363 | − | AGACCUUCCCGAGGAGUGCA | 20 | 749 |
| HSV1-UL19-364 | − | AGGAGUGCAUGGCCGUGUAC | 20 | 750 |
| HSV1-UL19-365 | − | GGAGUGCAUGGCCGUGUACC | 20 | 751 |
| HSV1-UL19-366 | − | CAUGGCCGUGUACCGGGACC | 20 | 752 |
| HSV1-UL19-367 | − | GGCCGUGUACCGGGACCUGG | 20 | 753 |
| HSV1-UL19-368 | − | GGACCUGGUGGCGCACGUCG | 20 | 754 |
| HSV1-UL19-369 | − | GGUGGCGCACGUCGAGGCCC | 20 | 755 |
| HSV1-UL19-370 | − | CGUCGAGGCCCUGGCCCAGC | 20 | 756 |
| HSV1-UL19-371 | − | GUUGAUGACUUUACCCUGAC | 20 | 757 |
| HSV1-UL19-372 | − | UGACUUUACCCUGACCGGCC | 20 | 758 |
| HSV1-UL19-373 | − | UACCCUGACCGGCCCGGAGC | 20 | 759 |
| HSV1-UL19-374 | − | ACCCUGACCGGCCCGGAGCU | 20 | 760 |
| HSV1-UL19-375 | − | CUGACCGGCCCGGAGCUGGG | 20 | 761 |
| HSV1-UL19-376 | − | UGACCGGCCCGGAGCUGGGC | 20 | 762 |
| HSV1-UL19-377 | − | CGGCCCGGAGCUGGGCGGGC | 20 | 763 |
| HSV1-UL19-378 | − | GAAUCACCUAAUGCGAGACC | 20 | 764 |
| HSV1-UL19-379 | − | CGCUGCUGCCACCCCUCGUG | 20 | 765 |
| HSV1-UL19-380 | − | GCUGCUGCCACCCCUCGUGU | 20 | 766 |
| HSV1-UL19-381 | − | GGGACUGUGACGCCCUGAUG | 20 | 767 |
| HSV1-UL19-382 | − | UGACGCCCUGAUGCGGCGCG | 20 | 768 |
| HSV1-UL19-383 | − | CCUGAUGCGGCGCGCGGCCC | 20 | 769 |
| HSV1-UL19-384 | − | UGGACCGCCAUCGCGACUGC | 20 | 770 |
| HSV1-UL19-385 | − | GGACCGCCAUCGCGACUGCC | 20 | 771 |
| HSV1-UL19-386 | − | UCGCGACUGCCGGGUUAGCG | 20 | 772 |
| HSV1-UL19-387 | − | CGCGACUGCCGGGUUAGCGC | 20 | 773 |
| HSV1-UL19-388 | − | GCGACUGCCGGGUUAGCGCG | 20 | 774 |
| HSV1-UL19-389 | − | CGACUGCCGGGUUAGCGCGG | 20 | 775 |
| HSV1-UL19-390 | − | GACUGCCGGGUUAGCGCGGG | 20 | 776 |
| HSV1-UL19-391 | − | GGGCCACGACCCCGUGUACG | 20 | 777 |
| HSV1-UL19-392 | − | CCACGACCCCGUGUACGCGG | 20 | 778 |
| HSV1-UL19-393 | − | GUACGCGGCGGCAUGUAACG | 20 | 779 |
| HSV1-UL19-394 | − | GGCAUGUAACGUGGCGACCG | 20 | 780 |
| HSV1-UL19-395 | − | GCGGACUUCAACCGCAACGA | 20 | 781 |
| HSV1-UL19-396 | − | CCAGCUGCUGCACAACACCC | 20 | 782 |
| HSV1-UL19-397 | − | CAACACCCAGGCCCGAGCCG | 20 | 783 |
| HSV1-UL19-398 | − | GGCCCGAGCCGCGGACGCCG | 20 | 784 |
| HSV1-UL19-399 | − | CCGCGGACGCCGCGGAUGAC | 20 | 785 |
| HSV1-UL19-400 | − | CCGCGGAUGACCGGCCGCAC | 20 | 786 |
| HSV1-UL19-401 | − | CGCGGAUGACCGGCCGCACC | 20 | 787 |
| HSV1-UL19-402 | − | GCGGAUGACCGGCCGCACCG | 20 | 788 |
| HSV1-UL19-403 | − | CGGAUGACCGGCCGCACCGG | 20 | 789 |
| HSV1-UL19-404 | − | GGAUGACCGGCCGCACCGGG | 20 | 790 |
| HSV1-UL19-405 | − | UGACCGGCCGCACCGGGGGG | 20 | 791 |
| HSV1-UL19-406 | − | GGCCGCACCGGGGGGCGGAC | 20 | 792 |
| HSV1-UL19-407 | − | CAAGAUUUACUACUACGUGA | 20 | 793 |
| HSV1-UL19-408 | − | UGAUGGUGCCCGCCUUCUCG | 20 | 794 |
| HSV1-UL19-409 | − | GAUGGUGCCCGCCUUCUCGC | 20 | 795 |
| HSV1-UL19-410 | − | AUGGUGCCCGCCUUCUCGCG | 20 | 796 |
| HSV1-UL19-411 | − | GCGGGGCCGCUGCUGCACCG | 20 | 797 |
| HSV1-UL19-412 | − | CGGGGCCGCUGCUGCACCGC | 20 | 798 |
| HSV1-UL19-413 | − | GGGGCCGCUGCUGCACCGCG | 20 | 799 |
| HSV1-UL19-414 | − | GGGCCGCUGCUGCACCGCGG | 20 | 800 |
| HSV1-UL19-415 | − | AUACGCCACCCUGCAGAACA | 20 | 801 |
| HSV1-UL19-416 | − | CGCCACCCUGCAGAACAUGG | 20 | 802 |
| HSV1-UL19-417 | − | CCUGCAGAACAUGGUGGUCC | 20 | 803 |
| HSV1-UL19-418 | − | GUGGUCCCGGAGAUCGCCCC | 20 | 804 |
| HSV1-UL19-419 | − | CCCGGAGAUCGCCCCCGGCG | 20 | 805 |
| HSV1-UL19-420 | − | GUGCCCCAGCGACCCCGUGA | 20 | 806 |
| HSV1-UL19-421 | − | CCCCGCGCACCCCCUGCACC | 20 | 807 |
| HSV1-UL19-422 | − | CCCCCUGCACCCGGCCAAUC | 20 | 808 |
| HSV1-UL19-423 | − | CCUGCACCCGGCCAAUCUGG | 20 | 809 |
| HSV1-UL19-424 | − | GGCCAAUCUGGUGGCCAACA | 20 | 810 |
| HSV1-UL19-425 | − | GUCAACGCCAUGUUUCACAA | 20 | 811 |
| HSV1-UL19-426 | − | UCAACGCCAUGUUUCACAAC | 20 | 812 |
| HSV1-UL19-427 | − | CAUGUUUCACAACGGGCGCG | 20 | 813 |
| HSV1-UL19-428 | − | UCACAACGGGCGCGUGGUAG | 20 | 814 |
| HSV1-UL19-429 | − | AACGGGCGCGUGGUAGUGGA | 20 | 815 |
| HSV1-UL19-430 | − | ACGGGCGCGUGGUAGUGGAC | 20 | 816 |
| HSV1-UL19-431 | − | GCCCGCCAUGCUCACGCUGC | 20 | 817 |
| HSV1-UL19-432 | − | CAUGCUCACGCUGCAGGUGC | 20 | 818 |
| HSV1-UL19-433 | − | GCAGGUGCUGGCCCACAACA | 20 | 819 |
| HSV1-UL19-434 | − | CAACAUGGCCGAACGCACAA | 20 | 820 |
| HSV1-UL19-435 | − | CACAACGGCGCUGCUCUGCU | 20 | 821 |
| HSV1-UL19-436 | − | AACGGCGCUGCUCUGCUCGG | 20 | 822 |
| HSV1-UL19-437 | − | CUGCUCGGCGGCGCCCGACG | 20 | 823 |
| HSV1-UL19-438 | − | UGCUCGGCGGCGCCCGACGC | 20 | 824 |
| HSV1-UL19-439 | − | ACCAACAUGCGCAUAUUCGA | 20 | 825 |
| HSV1-UL19-440 | − | CCAACAUGCGCAUAUUCGAC | 20 | 826 |
| HSV1-UL19-441 | − | CAACAUGCGCAUAUUCGACG | 20 | 827 |
| HSV1-UL19-442 | − | UUCGACGGGGCGUUGCACGC | 20 | 828 |
| HSV1-UL19-443 | − | GCACGCCGGAAUCCUGCUGA | 20 | 829 |
| HSV1-UL19-444 | − | GCUGAUGGCCCCCCAGCAUC | 20 | 830 |
| HSV1-UL19-445 | − | CUGGACCAUACCAUCCAAAA | 20 | 831 |
| HSV1-UL19-446 | − | CCCGUCCACGCGCUGUUCGC | 20 | 832 |
| HSV1-UL19-447 | − | CCGUCCACGCGCUGUUCGCC | 20 | 833 |
| HSV1-UL19-448 | − | CGUCCACGCGCUGUUCGCCG | 20 | 834 |
| HSV1-UL19-449 | − | GUUCGCCGGGGCCGACCACG | 20 | 835 |
| HSV1-UL19-450 | − | GAACGCGCCCAAUUUCCCCC | 20 | 836 |
| HSV1-UL19-451 | − | CGGCCCUGCGCGACCUGUCG | 20 | 837 |
| HSV1-UL19-452 | − | CCUGCGCGACCUGUCGCGGC | 20 | 838 |
| HSV1-UL19-453 | − | CCUGUCGCGGCAGGUCCCCC | 20 | 839 |
| HSV1-UL19-454 | − | GCAGGUCCCCCUGGUCCCCC | 20 | 840 |
| HSV1-UL19-455 | − | CCCCCUGGUCCCCCCGGCUC | 20 | 841 |
| HSV1-UL19-456 | − | CCCCUGGUCCCCCCGGCUCU | 20 | 842 |
| HSV1-UL19-457 | − | CCCUGGUCCCCCCGGCUCUG | 20 | 843 |
| HSV1-UL19-458 | − | CCUGGUCCCCCCGGCUCUGG | 20 | 844 |
| HSV1-UL19-459 | − | GCAGCACGUCCGCGAGAGCG | 20 | 845 |
| HSV1-UL19-460 | − | CACGUCCGCGAGAGCGCGGC | 20 | 846 |
| HSV1-UL19-461 | − | ACGUCCGCGAGAGCGCGGCC | 20 | 847 |
| HSV1-UL19-462 | − | CGUCCGCGAGAGCGCGGCCG | 20 | 848 |
| HSV1-UL19-463 | − | CGCGCUGACCUACGCGCUCA | 20 | 849 |
| HSV1-UL19-464 | − | GCUGACCUACGCGCUCAUGG | 20 | 850 |
| HSV1-UL19-465 | − | CUGACCUACGCGCUCAUGGC | 20 | 851 |
| HSV1-UL19-466 | − | UGACCUACGCGCUCAUGGCG | 20 | 852 |
| HSV1-UL19-467 | − | GUACUUCAAGAUCAGUCCCG | 20 | 853 |
| HSV1-UL19-468 | − | CUUGCAUCAUCAGCUCAAGA | 20 | 854 |
| HSV1-UL19-469 | − | UUGCAUCAUCAGCUCAAGAC | 20 | 855 |
| HSV1-UL19-470 | − | CUCAAGACGGGCCUCCAUCC | 20 | 856 |
| HSV1-UL19-471 | − | UCAAGACGGGCCUCCAUCCC | 20 | 857 |
| HSV1-UL19-472 | − | ACGGGCCUCCAUCCCGGGUU | 20 | 858 |
| HSV1-UL19-473 | − | CGGGCCUCCAUCCCGGGUUU | 20 | 859 |
| HSV1-UL19-474 | − | UGGGUUCACCGUCGUCCGAC | 20 | 860 |
| HSV1-UL19-475 | − | GACUGAGAACGUGCUGUUCU | 20 | 861 |
| HSV1-UL19-476 | − | GCUGUUCUCGGAGCGCGCGU | 20 | 862 |
| HSV1-UL19-477 | − | GUUCUCGGAGCGCGCGUCGG | 20 | 863 |
| HSV1-UL19-478 | − | CGCGUCGGAGGCGUACUUCC | 20 | 864 |
| HSV1-UL19-479 | − | GCGUCGGAGGCGUACUUCCU | 20 | 865 |
| HSV1-UL19-480 | − | GUACUUCCUGGGCCAGCUCC | 20 | 866 |
| HSV1-UL19-481 | − | CUUCCUGGGCCAGCUCCAGG | 20 | 867 |
| HSV1-UL19-482 | − | UGGGCCAGCUCCAGGUGGCC | 20 | 868 |
| HSV1-UL19-483 | − | CAGGUGGCCCGGCACGAAAC | 20 | 869 |
| HSV1-UL19-484 | − | GUGGCCCGGCACGAAACUGG | 20 | 870 |
| HSV1-UL19-485 | − | UGGCCCGGCACGAAACUGGC | 20 | 871 |
| HSV1-UL19-486 | − | GGCCCGGCACGAAACUGGCG | 20 | 872 |
| HSV1-UL19-487 | − | GCCCGGCACGAAACUGGCGG | 20 | 873 |
| HSV1-UL19-488 | − | CCCGGCACGAAACUGGCGGG | 20 | 874 |
| HSV1-UL19-489 | − | CCGGCACGAAACUGGCGGGG | 20 | 875 |
| HSV1-UL19-490 | − | CACCCAGCCGCGCGCGAACG | 20 | 876 |
| HSV1-UL19-491 | − | GCCGCGCGCGAACGUGGACC | 20 | 877 |
| HSV1-UL19-492 | − | CCGCGCGCGAACGUGGACCU | 20 | 878 |
| HSV1-UL19-493 | − | CGCGAACGUGGACCUGGGCG | 20 | 879 |
| HSV1-UL19-494 | − | GCGAACGUGGACCUGGGCGU | 20 | 880 |
| HSV1-UL19-495 | − | CGUGGGCUACACCGCCGUCG | 20 | 881 |
| HSV1-UL19-496 | − | CUACACCGCCGUCGUGGCCA | 20 | 882 |
| HSV1-UL19-497 | − | CCGCAACCCCGUCACCGACA | 20 | 883 |
| HSV1-UL19-498 | − | CGCAACCCCGUCACCGACAU | 20 | 884 |
| HSV1-UL19-499 | − | CCUUCCCCAAAACUUUUACC | 20 | 885 |
| HSV1-UL19-500 | − | CUUCCCCAAAACUUUUACCU | 20 | 886 |
| HSV1-UL19-501 | − | CAAAACUUUUACCUGGGCCG | 20 | 887 |
| HSV1-UL19-502 | − | AAAACUUUUACCUGGGCCGC | 20 | 888 |
| HSV1-UL19-503 | − | AAACUUUUACCUGGGCCGCG | 20 | 889 |
| HSV1-UL19-504 | − | CCGCGGGGCUCCCCCUCUCC | 20 | 890 |
| HSV1-UL19-505 | − | UCCCCCUCUCCUGGACAACG | 20 | 891 |
| HSV1-UL19-506 | − | ACGCGGCAGCCGUGUACCUG | 20 | 892 |
| HSV1-UL19-507 | − | AGCCGUGUACCUGCGGAACG | 20 | 893 |
| HSV1-UL19-508 | − | GUACCUGCGGAACGCGGUCG | 20 | 894 |
| HSV1-UL19-509 | − | CCUGCGGAACGCGGUCGUGG | 20 | 895 |
| HSV1-UL19-510 | − | CUGCGGAACGCGGUCGUGGC | 20 | 896 |
| HSV1-UL19-511 | − | GGUCGUGGCGGGAAACCGCC | 20 | 897 |
| HSV1-UL19-512 | − | GUCGUGGCGGGAAACCGCCU | 20 | 898 |
| HSV1-UL19-513 | − | UCGUGGCGGGAAACCGCCUG | 20 | 899 |
| HSV1-UL19-514 | − | GGCGGGAAACCGCCUGGGGC | 20 | 900 |
| HSV1-UL19-515 | − | GCCCAGCCCGUCCCCGUGUU | 20 | 901 |
| HSV1-UL19-516 | − | CCCAGCCCGUCCCCGUGUUC | 20 | 902 |
| HSV1-UL19-517 | − | CCCCGUGUUCGGGUGCGCCC | 20 | 903 |
| HSV1-UL19-518 | − | UCGGGUGCGCCCAGGUGCCG | 20 | 904 |
| HSV1-UL19-519 | − | GCCCAGGUGCCGCGGCGCGC | 20 | 905 |
| HSV1-UL19-520 | − | CCCAGGUGCCGCGGCGCGCA | 20 | 906 |
| HSV1-UL19-521 | − | GGUGCCGCGGCGCGCAGGGA | 20 | 907 |
| HSV1-UL19-522 | − | CGGCGCGCAGGGAUGGACCA | 20 | 908 |
| HSV1-UL19-523 | − | CGCAGGGAUGGACCACGGCC | 20 | 909 |
| HSV1-UL19-524 | − | CCGACGUCAACUACUUCCGC | 20 | 910 |
| HSV1-UL19-525 | − | UCCGCCGGCCCUGCAACCCC | 20 | 911 |
| HSV1-UL19-526 | − | CCGCCGGCCCUGCAACCCCC | 20 | 912 |
| HSV1-UL19-527 | − | CGCCGGCCCUGCAACCCCCG | 20 | 913 |
| HSV1-UL19-528 | − | AACCCCCGGGGACGCGCCGC | 20 | 914 |
| HSV1-UL19-529 | − | ACCCCCGGGGACGCGCCGCC | 20 | 915 |
| HSV1-UL19-530 | − | CCCCCGGGGACGCGCCGCCG | 20 | 916 |
| HSV1-UL19-531 | − | CCCCGGGGACGCGCCGCCGG | 20 | 917 |
| HSV1-UL19-532 | − | CGCCGCCGGGGGCGUUUACG | 20 | 918 |
| HSV1-UL19-533 | − | GCCGCCGGGGGCGUUUACGC | 20 | 919 |
| HSV1-UL19-534 | − | CCGCCGGGGGCGUUUACGCG | 20 | 920 |
| HSV1-UL19-535 | − | CGCCGGGGGCGUUUACGCGG | 20 | 921 |
| HSV1-UL19-536 | − | GGGCGUUUACGCGGGGGACA | 20 | 922 |
| HSV1-UL19-537 | − | CGUUUACGCGGGGGACAAGG | 20 | 923 |
| HSV1-UL19-538 | − | GUUUACGCGGGGGACAAGGA | 20 | 924 |
| HSV1-UL19-539 | − | UUUACGCGGGGGACAAGGAG | 20 | 925 |
| HSV1-UL19-540 | − | UUACGCGGGGGACAAGGAGG | 20 | 926 |
| HSV1-UL19-541 | − | ACCGCCCUCAUGUACGACCA | 20 | 927 |
| HSV1-UL19-542 | − | ACGGCCAGAGCGACCCGUCC | 20 | 928 |
| HSV1-UL19-543 | − | CGGCCAGAGCGACCCGUCCC | 20 | 929 |
| HSV1-UL19-544 | − | CGACCCGUCCCGGGCCUUCG | 20 | 930 |
| HSV1-UL19-545 | − | GUCCCGGGCCUUCGCGGCCA | 20 | 931 |
| HSV1-UL19-546 | − | UCGCGGCCACGGCCAACCCG | 20 | 932 |
| HSV1-UL19-547 | − | CGCGGCCACGGCCAACCCGU | 20 | 933 |
| HSV1-UL19-548 | − | GCGUCGCAGCGAUUUUCGUA | 20 | 934 |
| HSV1-UL19-549 | − | CGUCGCAGCGAUUUUCGUAC | 20 | 935 |
| HSV1-UL19-550 | − | GUCGCAGCGAUUUUCGUACG | 20 | 936 |
| HSV1-UL19-551 | − | UACGGGGACCUGCUCUAUAA | 20 | 937 |
| HSV1-UL19-552 | − | ACGGGGACCUGCUCUAUAAC | 20 | 938 |
| HSV1-UL19-553 | − | CGGGGACCUGCUCUAUAACG | 20 | 939 |
| HSV1-UL19-554 | − | AACGGGGCCUACCACCUCAA | 20 | 940 |
| HSV1-UL19-555 | − | ACGGGGCCUACCACCUCAAC | 20 | 941 |
| HSV1-UL19-556 | − | CGGGGCCUACCACCUCAACG | 20 | 942 |
| HSV1-UL19-557 | − | CCACCUCAACGGGGCCUCGC | 20 | 943 |
| HSV1-UL19-558 | − | CUGCUUUAAGUUCUUUACGU | 20 | 944 |
| HSV1-UL19-559 | − | CGCCGCCAAACAUCGCUGCC | 20 | 945 |
| HSV1-UL19-560 | − | CUGCCUGGAGCGCCUGAUCG | 20 | 946 |
| HSV1-UL19-561 | − | GGAGCGCCUGAUCGUGGAGA | 20 | 947 |
| HSV1-UL19-562 | − | GAGCGCCUGAUCGUGGAGAC | 20 | 948 |
| HSV1-UL19-563 | − | CCUGAUCGUGGAGACGGGUU | 20 | 949 |
| HSV1-UL19-564 | − | GAUCGUGGAGACGGGUUCGG | 20 | 950 |
| HSV1-UL19-565 | − | GACGGGUUCGGCGGUGUCCA | 20 | 951 |
| HSV1-UL19-566 | − | CGUACAGUUUAAGCGCCCCC | 20 | 952 |
| HSV1-UL19-567 | − | GUACAGUUUAAGCGCCCCCC | 20 | 953 |
| HSV1-UL19-568 | − | UACAGUUUAAGCGCCCCCCG | 20 | 954 |
| HSV1-UL19-569 | − | CCCGGGGUGCCGCGAACUCG | 20 | 955 |
| HSV1-UL19-570 | − | GGGGUGCCGCGAACUCGUGG | 20 | 956 |
| HSV1-UL19-571 | − | GAACUCGUGGAGGACCCGUG | 20 | 957 |
| HSV1-UL19-572 | − | GGACCCGUGUGGCCUGUUUC | 20 | 958 |
| HSV1-UL19-573 | − | CCCGUGUGGCCUGUUUCAGG | 20 | 959 |
| HSV1-UL19-574 | − | CUCCUCCGCAGUGCCCGCAA | 20 | 960 |
| HSV1-UL19-575 | − | UCCUCCGCAGUGCCCGCAAC | 20 | 961 |
| HSV1-UL19-576 | − | CCUCCGCAGUGCCCGCAACG | 20 | 962 |
| HSV1-UL19-577 | − | GCAACGGGGAAGCCCACGCG | 20 | 963 |
| HSV1-UL19-578 | − | CAACGGGGAAGCCCACGCGC | 20 | 964 |
| HSV1-UL19-579 | − | CUAUGACGCAUCCCCGCUCA | 20 | 965 |
| HSV1-UL19-580 | − | UAUGACGCAUCCCCGCUCAA | 20 | 966 |
| HSV1-UL19-581 | − | CGCAUCCCCGCUCAAGGGAC | 20 | 967 |
| HSV1-UL19-582 | − | AACGUCCAGGCCGUCCU | 17 | 968 |
| HSV1-UL19-583 | − | ACGUCCAGGCCGUCCUC | 17 | 969 |
| HSV1-UL19-584 | − | CGUCCAGGCCGUCCUCG | 17 | 970 |
| HSV1-UL19-585 | − | CUCGGGGCGUUUGAGCG | 17 | 971 |
| HSV1-UL19-586 | − | GGCGUUUGAGCGCGGCA | 17 | 972 |
| HSV1-UL19-587 | − | GAUGCUGCACGUGCUGC | 17 | 973 |
| HSV1-UL19-588 | − | GCACGUGCUGCUGGAAA | 17 | 974 |
| HSV1-UL19-589 | − | GGAAAAGGCGCCGCCCC | 17 | 975 |
| HSV1-UL19-590 | − | GCCGAUGCAACGAUACC | 17 | 976 |
| HSV1-UL19-591 | − | CAACGAUACCUGGACAA | 17 | 977 |
| HSV1-UL19-592 | − | CCUGGACAACGGCCGCC | 17 | 978 |
| HSV1-UL19-593 | − | ACGGCCGCCUGGCCACC | 17 | 979 |
| HSV1-UL19-594 | − | CGGCCGCCUGGCCACCA | 17 | 980 |
| HSV1-UL19-595 | − | CCGCCUGGCCACCAGGG | 17 | 981 |
| HSV1-UL19-596 | − | UGGCCACCAGGGUGGCC | 17 | 982 |
| HSV1-UL19-597 | − | GGCCACCAGGGUGGCCC | 17 | 983 |
| HSV1-UL19-598 | − | GGUGGCCCGGGCGACCC | 17 | 984 |
| HSV1-UL19-599 | − | CGAGACGAGCUUUUUCC | 17 | 985 |
| HSV1-UL19-600 | − | GAGACGAGCUUUUUCCU | 17 | 986 |
| HSV1-UL19-601 | − | GAGCUUUUUCCUGGGCA | 17 | 987 |
| HSV1-UL19-602 | − | CUUUUUCCUGGGCAAGG | 17 | 988 |
| HSV1-UL19-603 | − | UUUUUCCUGGGCAAGGC | 17 | 989 |
| HSV1-UL19-604 | − | GCAAGGCGGGCCACCGC | 17 | 990 |
| HSV1-UL19-605 | − | CAAGGCGGGCCACCGCC | 17 | 991 |
| HSV1-UL19-606 | − | GGCGGGCCACCGCCGGG | 17 | 992 |
| HSV1-UL19-607 | − | CCGCCGGGAGGCCGUCG | 17 | 993 |
| HSV1-UL19-608 | − | GGGAGGCCGUCGAGGCC | 17 | 994 |
| HSV1-UL19-609 | − | CGUCGAGGCCUGGCUCG | 17 | 995 |
| HSV1-UL19-610 | − | GCUCGUGGACCUCACCA | 17 | 996 |
| HSV1-UL19-611 | − | GGCCACGCAGCCCUCCG | 17 | 997 |
| HSV1-UL19-612 | − | ACGCAUGCCGACACGCG | 17 | 998 |
| HSV1-UL19-613 | − | CGCAUGCCGACACGCGC | 17 | 999 |
| HSV1-UL19-614 | − | AUGCCGACACGCGCGGG | 17 | 1000 |
| HSV1-UL19-615 | − | CGACACGCGCGGGCGGC | 17 | 1001 |
| HSV1-UL19-616 | − | CGCGGGCGGCCGGUCGA | 17 | 1002 |
| HSV1-UL19-617 | − | GCGGGCGGCCGGUCGAC | 17 | 1003 |
| HSV1-UL19-618 | − | CGGGCGGCCGGUCGACG | 17 | 1004 |
| HSV1-UL19-619 | − | GCAGUCCUUCCUGAAAG | 17 | 1005 |
| HSV1-UL19-620 | − | GUCCUUCCUGAAAGUGG | 17 | 1006 |
| HSV1-UL19-621 | − | CACCGAAGCCGACGUGC | 17 | 1007 |
| HSV1-UL19-622 | − | GACGUGCCGGUGACGUA | 17 | 1008 |
| HSV1-UL19-623 | − | GGUGACGUACGGCGAGA | 17 | 1009 |
| HSV1-UL19-624 | − | GGCGAGAUGGUCCUGAA | 17 | 1010 |
| HSV1-UL19-625 | − | GCGAGAUGGUCCUGAAC | 17 | 1011 |
| HSV1-UL19-626 | − | CGAGAUGGUCCUGAACG | 17 | 1012 |
| HSV1-UL19-627 | − | CCUGAACGGGGCCAACC | 17 | 1013 |
| HSV1-UL19-628 | − | CGGGGCCAACCUGGUCA | 17 | 1014 |
| HSV1-UL19-629 | − | GGUCACGGCGCUCGUGA | 17 | 1015 |
| HSV1-UL19-630 | − | GUCACGGCGCUCGUGAU | 17 | 1016 |
| HSV1-UL19-631 | − | GGCGCUCGUGAUGGGCA | 17 | 1017 |
| HSV1-UL19-632 | − | CAAGGCCGUGCGAAGUC | 17 | 1018 |
| HSV1-UL19-633 | − | GCGAAGUCUGGACGACG | 17 | 1019 |
| HSV1-UL19-634 | − | CGAAGUCUGGACGACGU | 17 | 1020 |
| HSV1-UL19-635 | − | CGUGGGCCGCCACCUGC | 17 | 1021 |
| HSV1-UL19-636 | − | CCACCUGCUGGAGAUGC | 17 | 1022 |
| HSV1-UL19-637 | − | CCUGCUGGAGAUGCAGG | 17 | 1023 |
| HSV1-UL19-638 | − | AGCAGCUCGACCUGAAC | 17 | 1024 |
| HSV1-UL19-639 | − | CCUGAACCGGCAGACGC | 17 | 1025 |
| HSV1-UL19-640 | − | GACGACGCGCGUGCGCG | 17 | 1026 |
| HSV1-UL19-641 | − | GCGCGUGCGCGCGGAUC | 17 | 1027 |
| HSV1-UL19-642 | − | GCGGAUCUGGUGUCCAU | 17 | 1028 |
| HSV1-UL19-643 | − | GUCCAUCGGCGAGAAGC | 17 | 1029 |
| HSV1-UL19-644 | − | CGAGAAGCUGGUCUUUC | 17 | 1030 |
| HSV1-UL19-645 | − | GAAGCUGGUCUUUCUGG | 17 | 1031 |
| HSV1-UL19-646 | − | GGUCUUUCUGGAGGCCC | 17 | 1032 |
| HSV1-UL19-647 | − | CAACGUUCCCUACCCCC | 17 | 1033 |
| HSV1-UL19-648 | − | CGUUCCCUACCCCCUGG | 17 | 1034 |
| HSV1-UL19-649 | − | GUUCCCUACCCCCUGGU | 17 | 1035 |
| HSV1-UL19-650 | − | CCCCCUGGUGGGCGCCA | 17 | 1036 |
| HSV1-UL19-651 | − | GACGUUCGUCCUGCCCC | 17 | 1037 |
| HSV1-UL19-652 | − | ACGUUCGUCCUGCCCCU | 17 | 1038 |
| HSV1-UL19-653 | − | CCUGGGCCUGUUCAAUC | 17 | 1039 |
| HSV1-UL19-654 | − | CCUGUUCAAUCCGGUCA | 17 | 1040 |
| HSV1-UL19-655 | − | UCAAUCCGGUCAUGGAA | 17 | 1041 |
| HSV1-UL19-656 | − | CGGUUUGCCGCGCACGC | 17 | 1042 |
| HSV1-UL19-657 | − | GGUUUGCCGCGCACGCC | 17 | 1043 |
| HSV1-UL19-658 | − | GUUUGCCGCGCACGCCG | 17 | 1044 |
| HSV1-UL19-659 | − | GACCUAGUCCCCGCCCC | 17 | 1045 |
| HSV1-UL19-660 | − | CCCCGCCCCCGGCCACC | 17 | 1046 |
| HSV1-UL19-661 | − | CCCGCCAGCUGUUUUUU | 17 | 1047 |
| HSV1-UL19-662 | − | CCGCCAGCUGUUUUUUU | 17 | 1048 |
| HSV1-UL19-663 | − | CGCCAGCUGUUUUUUUG | 17 | 1049 |
| HSV1-UL19-664 | − | GCCAGCUGUUUUUUUGG | 17 | 1050 |
| HSV1-UL19-665 | − | GCUGUUUUUUUGGGGGA | 17 | 1051 |
| HSV1-UL19-666 | − | UUGGGGGAAGGACCGCC | 17 | 1052 |
| HSV1-UL19-667 | − | GGUGCUGCGCCUGUCUC | 17 | 1053 |
| HSV1-UL19-668 | − | UCUCUGGAACACGCGAU | 17 | 1054 |
| HSV1-UL19-669 | − | CUCUGGAACACGCGAUC | 17 | 1055 |
| HSV1-UL19-670 | − | GCUGAUGAACGUUGACG | 17 | 1056 |
| HSV1-UL19-671 | − | GAUGAACGUUGACGCGG | 17 | 1057 |
| HSV1-UL19-672 | − | AACGUUGACGCGGCGGU | 17 | 1058 |
| HSV1-UL19-673 | − | ACGUUGACGCGGCGGUC | 17 | 1059 |
| HSV1-UL19-674 | − | CGUUGACGCGGCGGUCG | 17 | 1060 |
| HSV1-UL19-675 | − | GUUGACGCGGCGGUCGG | 17 | 1061 |
| HSV1-UL19-676 | − | GAAGCCGCCAAUCCGUA | 17 | 1062 |
| HSV1-UL19-677 | − | AAGCCGCCAAUCCGUAC | 17 | 1063 |
| HSV1-UL19-678 | − | AGCCGCCAAUCCGUACG | 17 | 1064 |
| HSV1-UL19-679 | − | UCCGUACGGGGCGUACG | 17 | 1065 |
| HSV1-UL19-680 | − | GUACGGGGCGUACGUGG | 17 | 1066 |
| HSV1-UL19-681 | − | GGCGUACGUGGCGGCCC | 17 | 1067 |
| HSV1-UL19-682 | − | UACGUGGCGGCCCCGGC | 17 | 1068 |
| HSV1-UL19-683 | − | AGCUGUUUUUGAACGCC | 17 | 1069 |
| HSV1-UL19-684 | − | GCUGUUUUUGAACGCCU | 17 | 1070 |
| HSV1-UL19-685 | − | CUGUUUUUGAACGCCUG | 17 | 1071 |
| HSV1-UL19-686 | − | UGUUUUUGAACGCCUGG | 17 | 1072 |
| HSV1-UL19-687 | − | CGCCUGGGGGCAGCGCC | 17 | 1073 |
| HSV1-UL19-688 | − | GGGCAGCGCCUGGCCCA | 17 | 1074 |
| HSV1-UL19-689 | − | GGCAGCGCCUGGCCCAC | 17 | 1075 |
| HSV1-UL19-690 | − | AGCGCCUGGCCCACGGG | 17 | 1076 |
| HSV1-UL19-691 | − | GCGCCUGGCCCACGGGC | 17 | 1077 |
| HSV1-UL19-692 | − | CCCACGGGCGGGUCCGA | 17 | 1078 |
| HSV1-UL19-693 | − | CCACGGGCGGGUCCGAU | 17 | 1079 |
| HSV1-UL19-694 | − | GCGGGUCCGAUGGGUCG | 17 | 1080 |
| HSV1-UL19-695 | − | GUCCGAUGGGUCGCGGA | 17 | 1081 |
| HSV1-UL19-696 | − | GGAAGGCCAGAUGACCC | 17 | 1082 |
| HSV1-UL19-697 | − | GCCCGACAACGCCAACC | 17 | 1083 |
| HSV1-UL19-698 | − | CGCGUUCGACUUCUUUG | 17 | 1084 |
| HSV1-UL19-699 | − | GCGUUCGACUUCUUUGU | 17 | 1085 |
| HSV1-UL19-700 | − | CGUUCGACUUCUUUGUG | 17 | 1086 |
| HSV1-UL19-701 | − | GUUCGACUUCUUUGUGG | 17 | 1087 |
| HSV1-UL19-702 | − | CGACUUCUUUGUGGGGG | 17 | 1088 |
| HSV1-UL19-703 | − | UGUGGGGGUGGCCGACG | 17 | 1089 |
| HSV1-UL19-704 | − | GGCCGACGUGGAGCUGC | 17 | 1090 |
| HSV1-UL19-705 | − | GCCGACGUGGAGCUGCC | 17 | 1091 |
| HSV1-UL19-706 | − | CCGACGUGGAGCUGCCG | 17 | 1092 |
| HSV1-UL19-707 | − | CGACGUGGAGCUGCCGG | 17 | 1093 |
| HSV1-UL19-708 | − | GACGUGGAGCUGCCGGG | 17 | 1094 |
| HSV1-UL19-709 | − | ACGUGGAGCUGCCGGGG | 17 | 1095 |
| HSV1-UL19-710 | − | CGUGGAGCUGCCGGGGG | 17 | 1096 |
| HSV1-UL19-711 | − | GGGGGGGGACGUUCCCC | 17 | 1097 |
| HSV1-UL19-712 | − | GGGGACGUUCCCCCGGC | 17 | 1098 |
| HSV1-UL19-713 | − | CGUUCCCCCGGCCGGCC | 17 | 1099 |
| HSV1-UL19-714 | − | GUUCCCCCGGCCGGCCC | 17 | 1100 |
| HSV1-UL19-715 | − | UUCCCCCGGCCGGCCCG | 17 | 1101 |
| HSV1-UL19-716 | − | UCCCCCGGCCGGCCCGG | 17 | 1102 |
| HSV1-UL19-717 | − | CGGCCCGGGGGAGAUCC | 17 | 1103 |
| HSV1-UL19-718 | − | GGGAGAUCCAGGCCACC | 17 | 1104 |
| HSV1-UL19-719 | − | CCAGGCCACCUGGCGCG | 17 | 1105 |
| HSV1-UL19-720 | − | ACCUGGCGCGUGGUGAA | 17 | 1106 |
| HSV1-UL19-721 | − | GAACGGCAACUUGCCCC | 17 | 1107 |
| HSV1-UL19-722 | − | GCCCCUGGCGCUAUGUC | 17 | 1108 |
| HSV1-UL19-723 | − | CCUGGCGCUAUGUCCGG | 17 | 1109 |
| HSV1-UL19-724 | − | UAUGUCCGGCGGCGUUC | 17 | 1110 |
| HSV1-UL19-725 | − | AUGUCCGGCGGCGUUCC | 17 | 1111 |
| HSV1-UL19-726 | − | CGGCGUUCCGGGACGCC | 17 | 1112 |
| HSV1-UL19-727 | − | GGCGUUCCGGGACGCCC | 17 | 1113 |
| HSV1-UL19-728 | − | GCGUUCCGGGACGCCCG | 17 | 1114 |
| HSV1-UL19-729 | − | CCGGGACGCCCGGGGCC | 17 | 1115 |
| HSV1-UL19-730 | − | CGCCCGGGGCCUGGAGC | 17 | 1116 |
| HSV1-UL19-731 | − | GCCCGGGGCCUGGAGCU | 17 | 1117 |
| HSV1-UL19-732 | − | CCCGGGGCCUGGAGCUG | 17 | 1118 |
| HSV1-UL19-733 | − | CCGGGGCCUGGAGCUGG | 17 | 1119 |
| HSV1-UL19-734 | − | GGGCCUGGAGCUGGGGG | 17 | 1120 |
| HSV1-UL19-735 | − | GGCCUGGAGCUGGGGGU | 17 | 1121 |
| HSV1-UL19-736 | − | GGUGGGACGCCACGCCA | 17 | 1122 |
| HSV1-UL19-737 | − | ACCAUCGCCGCCGUUCG | 17 | 1123 |
| HSV1-UL19-738 | − | CCAUCGCCGCCGUUCGC | 17 | 1124 |
| HSV1-UL19-739 | − | CAUCGCCGCCGUUCGCG | 17 | 1125 |
| HSV1-UL19-740 | − | CGACGACCGCAACUACC | 17 | 1126 |
| HSV1-UL19-741 | − | CGACCGCAACUACCCGG | 17 | 1127 |
| HSV1-UL19-742 | − | GGUGUUUUACCUGCUGC | 17 | 1128 |
| HSV1-UL19-743 | − | CUGCAGGCCGCCAUACA | 17 | 1129 |
| HSV1-UL19-744 | − | GCACGUCUUCUGCGCCC | 17 | 1130 |
| HSV1-UL19-745 | − | UCUUCUGCGCCCUGGCC | 17 | 1131 |
| HSV1-UL19-746 | − | CGCCCUGGCCCGGCUCG | 17 | 1132 |
| HSV1-UL19-747 | − | AGUGCAUCACCAGCUAC | 17 | 1133 |
| HSV1-UL19-748 | − | GCUACUGGAACAACACG | 17 | 1134 |
| HSV1-UL19-749 | − | GAACAACACGCGGUGCG | 17 | 1135 |
| HSV1-UL19-750 | − | CGUGAACGACUACUCGC | 17 | 1136 |
| HSV1-UL19-751 | − | UACGUCGUGACCUACCU | 17 | 1137 |
| HSV1-UL19-752 | − | ACGUCGUGACCUACCUC | 17 | 1138 |
| HSV1-UL19-753 | − | CGUCGUGACCUACCUCG | 17 | 1139 |
| HSV1-UL19-754 | − | GUCGUGACCUACCUCGG | 17 | 1140 |
| HSV1-UL19-755 | − | CGGGGGAGACCUUCCCG | 17 | 1141 |
| HSV1-UL19-756 | − | CCUUCCCGAGGAGUGCA | 17 | 1142 |
| HSV1-UL19-757 | − | AGUGCAUGGCCGUGUAC | 17 | 1143 |
| HSV1-UL19-758 | − | GUGCAUGGCCGUGUACC | 17 | 1144 |
| HSV1-UL19-759 | − | GGCCGUGUACCGGGACC | 17 | 1145 |
| HSV1-UL19-760 | − | CGUGUACCGGGACCUGG | 17 | 1146 |
| HSV1-UL19-761 | − | CCUGGUGGCGCACGUCG | 17 | 1147 |
| HSV1-UL19-762 | − | GGCGCACGUCGAGGCCC | 17 | 1148 |
| HSV1-UL19-763 | − | CGAGGCCCUGGCCCAGC | 17 | 1149 |
| HSV1-UL19-764 | − | GAUGACUUUACCCUGAC | 17 | 1150 |
| HSV1-UL19-765 | − | CUUUACCCUGACCGGCC | 17 | 1151 |
| HSV1-UL19-766 | − | CCUGACCGGCCCGGAGC | 17 | 1152 |
| HSV1-UL19-767 | − | CUGACCGGCCCGGAGCU | 17 | 1153 |
| HSV1-UL19-768 | − | ACCGGCCCGGAGCUGGG | 17 | 1154 |
| HSV1-UL19-769 | − | CCGGCCCGGAGCUGGGC | 17 | 1155 |
| HSV1-UL19-770 | − | CCCGGAGCUGGGCGGGC | 17 | 1156 |
| HSV1-UL19-771 | − | UCACCUAAUGCGAGACC | 17 | 1157 |
| HSV1-UL19-772 | − | UGCUGCCACCCCUCGUG | 17 | 1158 |
| HSV1-UL19-773 | − | GCUGCCACCCCUCGUGU | 17 | 1159 |
| HSV1-UL19-774 | − | ACUGUGACGCCCUGAUG | 17 | 1160 |
| HSV1-UL19-775 | − | CGCCCUGAUGCGGCGCG | 17 | 1161 |
| HSV1-UL19-776 | − | GAUGCGGCGCGCGGCCC | 17 | 1162 |
| HSV1-UL19-777 | − | ACCGCCAUCGCGACUGC | 17 | 1163 |
| HSV1-UL19-778 | − | CCGCCAUCGCGACUGCC | 17 | 1164 |
| HSV1-UL19-779 | − | CGACUGCCGGGUUAGCG | 17 | 1165 |
| HSV1-UL19-780 | − | GACUGCCGGGUUAGCGC | 17 | 1166 |
| HSV1-UL19-781 | − | ACUGCCGGGUUAGCGCG | 17 | 1167 |
| HSV1-UL19-782 | − | CUGCCGGGUUAGCGCGG | 17 | 1168 |
| HSV1-UL19-783 | − | UGCCGGGUUAGCGCGGG | 17 | 1169 |
| HSV1-UL19-784 | − | CCACGACCCCGUGUACG | 17 | 1170 |
| HSV1-UL19-785 | − | CGACCCCGUGUACGCGG | 17 | 1171 |
| HSV1-UL19-786 | − | CGCGGCGGCAUGUAACG | 17 | 1172 |
| HSV1-UL19-787 | − | AUGUAACGUGGCGACCG | 17 | 1173 |
| HSV1-UL19-788 | − | GACUUCAACCGCAACGA | 17 | 1174 |
| HSV1-UL19-789 | − | GCUGCUGCACAACACCC | 17 | 1175 |
| HSV1-UL19-790 | − | CACCCAGGCCCGAGCCG | 17 | 1176 |
| HSV1-UL19-791 | − | CCGAGCCGCGGACGCCG | 17 | 1177 |
| HSV1-UL19-792 | − | CGGACGCCGCGGAUGAC | 17 | 1178 |
| HSV1-UL19-793 | − | CGGAUGACCGGCCGCAC | 17 | 1179 |
| HSV1-UL19-794 | − | GGAUGACCGGCCGCACC | 17 | 1180 |
| HSV1-UL19-795 | − | GAUGACCGGCCGCACCG | 17 | 1181 |
| HSV1-UL19-796 | − | AUGACCGGCCGCACCGG | 17 | 1182 |
| HSV1-UL19-797 | − | UGACCGGCCGCACCGGG | 17 | 1183 |
| HSV1-UL19-798 | − | CCGGCCGCACCGGGGGG | 17 | 1184 |
| HSV1-UL19-799 | − | CGCACCGGGGGGCGGAC | 17 | 1185 |
| HSV1-UL19-800 | − | GAUUUACUACUACGUGA | 17 | 1186 |
| HSV1-UL19-801 | − | UGGUGCCCGCCUUCUCG | 17 | 1187 |
| HSV1-UL19-802 | − | GGUGCCCGCCUUCUCGC | 17 | 1188 |
| HSV1-UL19-803 | − | GUGCCCGCCUUCUCGCG | 17 | 1189 |
| HSV1-UL19-804 | − | GGGCCGCUGCUGCACCG | 17 | 1190 |
| HSV1-UL19-805 | − | GGCCGCUGCUGCACCGC | 17 | 1191 |
| HSV1-UL19-806 | − | GCCGCUGCUGCACCGCG | 17 | 1192 |
| HSV1-UL19-807 | − | CCGCUGCUGCACCGCGG | 17 | 1193 |
| HSV1-UL19-808 | − | CGCCACCCUGCAGAACA | 17 | 1194 |
| HSV1-UL19-809 | − | CACCCUGCAGAACAUGG | 17 | 1195 |
| HSV1-UL19-810 | − | GCAGAACAUGGUGGUCC | 17 | 1196 |
| HSV1-UL19-811 | − | GUCCCGGAGAUCGCCCC | 17 | 1197 |
| HSV1-UL19-812 | − | GGAGAUCGCCCCCGGCG | 17 | 1198 |
| HSV1-UL19-813 | − | CCCCAGCGACCCCGUGA | 17 | 1199 |
| HSV1-UL19-814 | − | CGCGCACCCCCUGCACC | 17 | 1200 |
| HSV1-UL19-815 | − | CCUGCACCCGGCCAAUC | 17 | 1201 |
| HSV1-UL19-816 | − | GCACCCGGCCAAUCUGG | 17 | 1202 |
| HSV1-UL19-817 | − | CAAUCUGGUGGCCAACA | 17 | 1203 |
| HSV1-UL19-818 | − | AACGCCAUGUUUCACAA | 17 | 1204 |
| HSV1-UL19-819 | − | ACGCCAUGUUUCACAAC | 17 | 1205 |
| HSV1-UL19-820 | − | GUUUCACAACGGGCGCG | 17 | 1206 |
| HSV1-UL19-821 | − | CAACGGGCGCGUGGUAG | 17 | 1207 |
| HSV1-UL19-822 | − | GGGCGCGUGGUAGUGGA | 17 | 1208 |
| HSV1-UL19-823 | − | GGCGCGUGGUAGUGGAC | 17 | 1209 |
| HSV1-UL19-824 | − | CGCCAUGCUCACGCUGC | 17 | 1210 |
| HSV1-UL19-825 | − | GCUCACGCUGCAGGUGC | 17 | 1211 |
| HSV1-UL19-826 | − | GGUGCUGGCCCACAACA | 17 | 1212 |
| HSV1-UL19-827 | − | CAUGGCCGAACGCACAA | 17 | 1213 |
| HSV1-UL19-828 | − | AACGGCGCUGCUCUGCU | 17 | 1214 |
| HSV1-UL19-829 | − | GGCGCUGCUCUGCUCGG | 17 | 1215 |
| HSV1-UL19-830 | − | CUCGGCGGCGCCCGACG | 17 | 1216 |
| HSV1-UL19-831 | − | UCGGCGGCGCCCGACGC | 17 | 1217 |
| HSV1-UL19-832 | − | AACAUGCGCAUAUUCGA | 17 | 1218 |
| HSV1-UL19-833 | − | ACAUGCGCAUAUUCGAC | 17 | 1219 |
| HSV1-UL19-834 | − | CAUGCGCAUAUUCGACG | 17 | 1220 |
| HSV1-UL19-835 | − | GACGGGGCGUUGCACGC | 17 | 1221 |
| HSV1-UL19-836 | − | CGCCGGAAUCCUGCUGA | 17 | 1222 |
| HSV1-UL19-837 | − | GAUGGCCCCCCAGCAUC | 17 | 1223 |
| HSV1-UL19-838 | − | GACCAUACCAUCCAAAA | 17 | 1224 |
| HSV1-UL19-839 | − | GUCCACGCGCUGUUCGC | 17 | 1225 |
| HSV1-UL19-840 | − | UCCACGCGCUGUUCGCC | 17 | 1226 |
| HSV1-UL19-841 | − | CCACGCGCUGUUCGCCG | 17 | 1227 |
| HSV1-UL19-842 | − | CGCCGGGGCCGACCACG | 17 | 1228 |
| HSV1-UL19-843 | − | CGCGCCCAAUUUCCCCC | 17 | 1229 |
| HSV1-UL19-844 | − | CCCUGCGCGACCUGUCG | 17 | 1230 |
| HSV1-UL19-845 | − | GCGCGACCUGUCGCGGC | 17 | 1231 |
| HSV1-UL19-846 | − | GUCGCGGCAGGUCCCCC | 17 | 1232 |
| HSV1-UL19-847 | − | GGUCCCCCUGGUCCCCC | 17 | 1233 |
| HSV1-UL19-848 | − | CCUGGUCCCCCCGGCUC | 17 | 1234 |
| HSV1-UL19-849 | − | CUGGUCCCCCCGGCUCU | 17 | 1235 |
| HSV1-UL19-850 | − | UGGUCCCCCCGGCUCUG | 17 | 1236 |
| HSV1-UL19-851 | − | GGUCCCCCCGGCUCUGG | 17 | 1237 |
| HSV1-UL19-852 | − | GCACGUCCGCGAGAGCG | 17 | 1238 |
| HSV1-UL19-853 | − | GUCCGCGAGAGCGCGGC | 17 | 1239 |
| HSV1-UL19-854 | − | UCCGCGAGAGCGCGGCC | 17 | 1240 |
| HSV1-UL19-855 | − | CCGCGAGAGCGCGGCCG | 17 | 1241 |
| HSV1-UL19-856 | − | GCUGACCUACGCGCUCA | 17 | 1242 |
| HSV1-UL19-857 | − | GACCUACGCGCUCAUGG | 17 | 1243 |
| HSV1-UL19-858 | − | ACCUACGCGCUCAUGGC | 17 | 1244 |
| HSV1-UL19-859 | − | CCUACGCGCUCAUGGCG | 17 | 1245 |
| HSV1-UL19-860 | − | CUUCAAGAUCAGUCCCG | 17 | 1246 |
| HSV1-UL19-861 | − | GCAUCAUCAGCUCAAGA | 17 | 1247 |
| HSV1-UL19-862 | − | CAUCAUCAGCUCAAGAC | 17 | 1248 |
| HSV1-UL19-863 | − | AAGACGGGCCUCCAUCC | 17 | 1249 |
| HSV1-UL19-864 | − | AGACGGGCCUCCAUCCC | 17 | 1250 |
| HSV1-UL19-865 | − | GGCCUCCAUCCCGGGUU | 17 | 1251 |
| HSV1-UL19-866 | − | GCCUCCAUCCCGGGUUU | 17 | 1252 |
| HSV1-UL19-867 | − | GUUCACCGUCGUCCGAC | 17 | 1253 |
| HSV1-UL19-868 | − | UGAGAACGUGCUGUUCU | 17 | 1254 |
| HSV1-UL19-869 | − | GUUCUCGGAGCGCGCGU | 17 | 1255 |
| HSV1-UL19-870 | − | CUCGGAGCGCGCGUCGG | 17 | 1256 |
| HSV1-UL19-871 | − | GUCGGAGGCGUACUUCC | 17 | 1257 |
| HSV1-UL19-872 | − | UCGGAGGCGUACUUCCU | 17 | 1258 |
| HSV1-UL19-873 | − | CUUCCUGGGCCAGCUCC | 17 | 1259 |
| HSV1-UL19-874 | − | CCUGGGCCAGCUCCAGG | 17 | 1260 |
| HSV1-UL19-875 | − | GCCAGCUCCAGGUGGCC | 17 | 1261 |
| HSV1-UL19-876 | − | GUGGCCCGGCACGAAAC | 17 | 1262 |
| HSV1-UL19-877 | − | GCCCGGCACGAAACUGG | 17 | 1263 |
| HSV1-UL19-878 | − | CCCGGCACGAAACUGGC | 17 | 1264 |
| HSV1-UL19-879 | − | CCGGCACGAAACUGGCG | 17 | 1265 |
| HSV1-UL19-880 | − | CGGCACGAAACUGGCGG | 17 | 1266 |
| HSV1-UL19-881 | − | GGCACGAAACUGGCGGG | 17 | 1267 |
| HSV1-UL19-882 | − | GCACGAAACUGGCGGGG | 17 | 1268 |
| HSV1-UL19-883 | − | CCAGCCGCGCGCGAACG | 17 | 1269 |
| HSV1-UL19-884 | − | GCGCGCGAACGUGGACC | 17 | 1270 |
| HSV1-UL19-885 | − | CGCGCGAACGUGGACCU | 17 | 1271 |
| HSV1-UL19-886 | − | GAACGUGGACCUGGGCG | 17 | 1272 |
| HSV1-UL19-887 | − | AACGUGGACCUGGGCGU | 17 | 1273 |
| HSV1-UL19-888 | − | GGGCUACACCGCCGUCG | 17 | 1274 |
| HSV1-UL19-889 | − | CACCGCCGUCGUGGCCA | 17 | 1275 |
| HSV1-UL19-890 | − | CAACCCCGUCACCGACA | 17 | 1276 |
| HSV1-UL19-891 | − | AACCCCGUCACCGACAU | 17 | 1277 |
| HSV1-UL19-892 | − | UCCCCAAAACUUUUACC | 17 | 1278 |
| HSV1-UL19-893 | − | CCCCAAAACUUUUACCU | 17 | 1279 |
| HSV1-UL19-894 | − | AACUUUUACCUGGGCCG | 17 | 1280 |
| HSV1-UL19-895 | − | ACUUUUACCUGGGCCGC | 17 | 1281 |
| HSV1-UL19-896 | − | CUUUUACCUGGGCCGCG | 17 | 1282 |
| HSV1-UL19-897 | − | CGGGGCUCCCCCUCUCC | 17 | 1283 |
| HSV1-UL19-898 | − | CCCUCUCCUGGACAACG | 17 | 1284 |
| HSV1-UL19-899 | − | CGGCAGCCGUGUACCUG | 17 | 1285 |
| HSV1-UL19-900 | − | CGUGUACCUGCGGAACG | 17 | 1286 |
| HSV1-UL19-901 | − | CCUGCGGAACGCGGUCG | 17 | 1287 |
| HSV1-UL19-902 | − | GCGGAACGCGGUCGUGG | 17 | 1288 |
| HSV1-UL19-903 | − | CGGAACGCGGUCGUGGC | 17 | 1289 |
| HSV1-UL19-904 | − | CGUGGCGGGAAACCGCC | 17 | 1290 |
| HSV1-UL19-905 | − | GUGGCGGGAAACCGCCU | 17 | 1291 |
| HSV1-UL19-906 | − | UGGCGGGAAACCGCCUG | 17 | 1292 |
| HSV1-UL19-907 | − | GGGAAACCGCCUGGGGC | 17 | 1293 |
| HSV1-UL19-908 | − | CAGCCCGUCCCCGUGUU | 17 | 1294 |
| HSV1-UL19-909 | − | AGCCCGUCCCCGUGUUC | 17 | 1295 |
| HSV1-UL19-910 | − | CGUGUUCGGGUGCGCCC | 17 | 1296 |
| HSV1-UL19-911 | − | GGUGCGCCCAGGUGCCG | 17 | 1297 |
| HSV1-UL19-912 | − | CAGGUGCCGCGGCGCGC | 17 | 1298 |
| HSV1-UL19-913 | − | AGGUGCCGCGGCGCGCA | 17 | 1299 |
| HSV1-UL19-914 | − | GCCGCGGCGCGCAGGGA | 17 | 1300 |
| HSV1-UL19-915 | − | CGCGCAGGGAUGGACCA | 17 | 1301 |
| HSV1-UL19-916 | − | AGGGAUGGACCACGGCC | 17 | 1302 |
| HSV1-UL19-917 | − | ACGUCAACUACUUCCGC | 17 | 1303 |
| HSV1-UL19-918 | − | GCCGGCCCUGCAACCCC | 17 | 1304 |
| HSV1-UL19-919 | − | CCGGCCCUGCAACCCCC | 17 | 1305 |
| HSV1-UL19-920 | − | CGGCCCUGCAACCCCCG | 17 | 1306 |
| HSV1-UL19-921 | − | CCCCGGGGACGCGCCGC | 17 | 1307 |
| HSV1-UL19-922 | − | CCCGGGGACGCGCCGCC | 17 | 1308 |
| HSV1-UL19-923 | − | CCGGGGACGCGCCGCCG | 17 | 1309 |
| HSV1-UL19-924 | − | CGGGGACGCGCCGCCGG | 17 | 1310 |
| HSV1-UL19-925 | − | CGCCGGGGGCGUUUACG | 17 | 1311 |
| HSV1-UL19-926 | − | GCCGGGGGCGUUUACGC | 17 | 1312 |
| HSV1-UL19-927 | − | CCGGGGGCGUUUACGCG | 17 | 1313 |
| HSV1-UL19-928 | − | CGGGGGCGUUUACGCGG | 17 | 1314 |
| HSV1-UL19-929 | − | CGUUUACGCGGGGGACA | 17 | 1315 |
| HSV1-UL19-930 | − | UUACGCGGGGGACAAGG | 17 | 1316 |
| HSV1-UL19-931 | − | UACGCGGGGGACAAGGA | 17 | 1317 |
| HSV1-UL19-932 | − | ACGCGGGGGACAAGGAG | 17 | 1318 |
| HSV1-UL19-933 | − | CGCGGGGGACAAGGAGG | 17 | 1319 |
| HSV1-UL19-934 | − | GCCCUCAUGUACGACCA | 17 | 1320 |
| HSV1-UL19-935 | − | GCCAGAGCGACCCGUCC | 17 | 1321 |
| HSV1-UL19-936 | − | CCAGAGCGACCCGUCCC | 17 | 1322 |
| HSV1-UL19-937 | − | CCCGUCCCGGGCCUUCG | 17 | 1323 |
| HSV1-UL19-938 | − | CCGGGCCUUCGCGGCCA | 17 | 1324 |
| HSV1-UL19-939 | − | CGGCCACGGCCAACCCG | 17 | 1325 |
| HSV1-UL19-940 | − | GGCCACGGCCAACCCGU | 17 | 1326 |
| HSV1-UL19-941 | − | UCGCAGCGAUUUUCGUA | 17 | 1327 |
| HSV1-UL19-942 | − | CGCAGCGAUUUUCGUAC | 17 | 1328 |
| HSV1-UL19-943 | − | GCAGCGAUUUUCGUACG | 17 | 1329 |
| HSV1-UL19-944 | − | GGGGACCUGCUCUAUAA | 17 | 1330 |
| HSV1-UL19-945 | − | GGGACCUGCUCUAUAAC | 17 | 1331 |
| HSV1-UL19-946 | − | GGACCUGCUCUAUAACG | 17 | 1332 |
| HSV1-UL19-947 | − | GGGGCCUACCACCUCAA | 17 | 1333 |
| HSV1-UL19-948 | − | GGGCCUACCACCUCAAC | 17 | 1334 |
| HSV1-UL19-949 | − | GGCCUACCACCUCAACG | 17 | 1335 |
| HSV1-UL19-950 | − | CCUCAACGGGGCCUCGC | 17 | 1336 |
| HSV1-UL19-951 | − | CUUUAAGUUCUUUACGU | 17 | 1337 |
| HSV1-UL19-952 | − | CGCCAAACAUCGCUGCC | 17 | 1338 |
| HSV1-UL19-953 | − | CCUGGAGCGCCUGAUCG | 17 | 1339 |
| HSV1-UL19-954 | − | GCGCCUGAUCGUGGAGA | 17 | 1340 |
| HSV1-UL19-955 | − | CGCCUGAUCGUGGAGAC | 17 | 1341 |
| HSV1-UL19-956 | − | GAUCGUGGAGACGGGUU | 17 | 1342 |
| HSV1-UL19-957 | − | CGUGGAGACGGGUUCGG | 17 | 1343 |
| HSV1-UL19-958 | − | GGGUUCGGCGGUGUCCA | 17 | 1344 |
| HSV1-UL19-959 | − | ACAGUUUAAGCGCCCCC | 17 | 1345 |
| HSV1-UL19-960 | − | CAGUUUAAGCGCCCCCC | 17 | 1346 |
| HSV1-UL19-961 | − | AGUUUAAGCGCCCCCCG | 17 | 1347 |
| HSV1-UL19-962 | − | GGGGUGCCGCGAACUCG | 17 | 1348 |
| HSV1-UL19-963 | − | GUGCCGCGAACUCGUGG | 17 | 1349 |
| HSV1-UL19-964 | − | CUCGUGGAGGACCCGUG | 17 | 1350 |
| HSV1-UL19-965 | − | CCCGUGUGGCCUGUUUC | 17 | 1351 |
| HSV1-UL19-966 | − | GUGUGGCCUGUUUCAGG | 17 | 1352 |
| HSV1-UL19-967 | − | CUCCGCAGUGCCCGCAA | 17 | 1353 |
| HSV1-UL19-968 | − | UCCGCAGUGCCCGCAAC | 17 | 1354 |
| HSV1-UL19-969 | − | CCGCAGUGCCCGCAACG | 17 | 1355 |
| HSV1-UL19-970 | − | ACGGGGAAGCCCACGCG | 17 | 1356 |
| HSV1-UL19-971 | − | CGGGGAAGCCCACGCGC | 17 | 1357 |
| HSV1-UL19-972 | − | UGACGCAUCCCCGCUCA | 17 | 1358 |
| HSV1-UL19-973 | − | GACGCAUCCCCGCUCAA | 17 | 1359 |
| HSV1-UL19-974 | − | AUCCCCGCUCAAGGGAC | 17 | 1360 |
| HSV1-UL19-975 | + | CAAACGCCCCGAGGACGGCC | 20 | 1361 |
| HSV1-UL19-976 | + | GCGCUCAAACGCCCCGAGGA | 20 | 1362 |
| HSV1-UL19-977 | + | UGCCGCGCUCAAACGCCCCG | 20 | 1363 |
| HSV1-UL19-978 | + | CAGCACGUGCAGCAUCUGGU | 20 | 1364 |
| HSV1-UL19-979 | + | CCAGCAGCACGUGCAGCAUC | 20 | 1365 |
| HSV1-UL19-980 | + | GGCAACAACAAAGCCAGGGG | 20 | 1366 |
| HSV1-UL19-981 | + | AUCGGCAACAACAAAGCCAG | 20 | 1367 |
| HSV1-UL19-982 | + | CAUCGGCAACAACAAAGCCA | 20 | 1368 |
| HSV1-UL19-983 | + | GCAUCGGCAACAACAAAGCC | 20 | 1369 |
| HSV1-UL19-984 | + | UUGUCCAGGUAUCGUUGCAU | 20 | 1370 |
| HSV1-UL19-985 | + | UGGCCAGGCGGCCGUUGUCC | 20 | 1371 |
| HSV1-UL19-986 | + | GGGCCACCCUGGUGGCCAGG | 20 | 1372 |
| HSV1-UL19-987 | + | CCCGGGCCACCCUGGUGGCC | 20 | 1373 |
| HSV1-UL19-988 | + | GGUCGCCCGGGCCACCCUGG | 20 | 1374 |
| HSV1-UL19-989 | + | CAGGGUCGCCCGGGCCACCC | 20 | 1375 |
| HSV1-UL19-990 | + | CUCGGCGACCAGGGUCGCCC | 20 | 1376 |
| HSV1-UL19-991 | + | GCUCGGCGACCAGGGUCGCC | 20 | 1377 |
| HSV1-UL19-992 | + | UCGCUUUAGCUCGGCGACCA | 20 | 1378 |
| HSV1-UL19-993 | + | UUCGCUUUAGCUCGGCGACC | 20 | 1379 |
| HSV1-UL19-994 | + | GCAGAAGCUUCGCUUUAGCU | 20 | 1380 |
| HSV1-UL19-995 | + | GGCGGUGGCCCGCCUUGCCC | 20 | 1381 |
| HSV1-UL19-996 | + | CCUCGACGGCCUCCCGGCGG | 20 | 1382 |
| HSV1-UL19-997 | + | AGGCCUCGACGGCCUCCCGG | 20 | 1383 |
| HSV1-UL19-998 | + | GCCAGGCCUCGACGGCCUCC | 20 | 1384 |
| HSV1-UL19-999 | + | GUCCACGAGCCAGGCCUCGA | 20 | 1385 |
| HSV1-UL19-1000 | + | CGUGGUGAGGUCCACGAGCC | 20 | 1386 |
| HSV1-UL19-1001 | + | AGGGCUGCGUGGCCGUGGUG | 20 | 1387 |
| HSV1-UL19-1002 | + | CACGGAGGGCUGCGUGGCCG | 20 | 1388 |
| HSV1-UL19-1003 | + | CACGGCCACGGAGGGCUGCG | 20 | 1389 |
| HSV1-UL19-1004 | + | AGACGGGGCACGGCCACGGA | 20 | 1390 |
| HSV1-UL19-1005 | + | CAGACGGGGCACGGCCACGG | 20 | 1391 |
| HSV1-UL19-1006 | + | CGUCAGACGGGGCACGGCCA | 20 | 1392 |
| HSV1-UL19-1007 | + | GGCAUGCGUCAGACGGGGCA | 20 | 1393 |
| HSV1-UL19-1008 | + | GUGUCGGCAUGCGUCAGACG | 20 | 1394 |
| HSV1-UL19-1009 | + | CGUGUCGGCAUGCGUCAGAC | 20 | 1395 |
| HSV1-UL19-1010 | + | GCGUGUCGGCAUGCGUCAGA | 20 | 1396 |
| HSV1-UL19-1011 | + | GACCGGCCGCCCGCGCGUGU | 20 | 1397 |
| HSV1-UL19-1012 | + | GUGACGAGCACCCCGUCGAC | 20 | 1398 |
| HSV1-UL19-1013 | + | GCGCUGUUUGAUCGGGGCGG | 20 | 1399 |
| HSV1-UL19-1014 | + | GAGGCGCUGUUUGAUCGGGG | 20 | 1400 |
| HSV1-UL19-1015 | + | CAGGAGGCGCUGUUUGAUCG | 20 | 1401 |
| HSV1-UL19-1016 | + | GCAGGAGGCGCUGUUUGAUC | 20 | 1402 |
| HSV1-UL19-1017 | + | UGCAGGAGGCGCUGUUUGAU | 20 | 1403 |
| HSV1-UL19-1018 | + | CUUUCAGGAAGGACUGCAGG | 20 | 1404 |
| HSV1-UL19-1019 | + | CCACUUUCAGGAAGGACUGC | 20 | 1405 |
| HSV1-UL19-1020 | + | GGUGUCCUCCACUUUCAGGA | 20 | 1406 |
| HSV1-UL19-1021 | + | CUUCGGUGUCCUCCACUUUC | 20 | 1407 |
| HSV1-UL19-1022 | + | CGUCACCGGCACGUCGGCUU | 20 | 1408 |
| HSV1-UL19-1023 | + | GCCGUACGUCACCGGCACGU | 20 | 1409 |
| HSV1-UL19-1024 | + | ACCAUCUCGCCGUACGUCAC | 20 | 1410 |
| HSV1-UL19-1025 | + | UGACCAGGUUGGCCCCGUUC | 20 | 1411 |
| HSV1-UL19-1026 | + | CACGAGCGCCGUGACCAGGU | 20 | 1412 |
| HSV1-UL19-1027 | + | CCAUCACGAGCGCCGUGACC | 20 | 1413 |
| HSV1-UL19-1028 | + | CACGUCGUCCAGACUUCGCA | 20 | 1414 |
| HSV1-UL19-1029 | + | CCUGCAUCUCCAGCAGGUGG | 20 | 1415 |
| HSV1-UL19-1030 | + | CCUCCUGCAUCUCCAGCAGG | 20 | 1416 |
| HSV1-UL19-1031 | + | GCUCCUCCUGCAUCUCCAGC | 20 | 1417 |
| HSV1-UL19-1032 | + | CGUCCAGCGUCUGCCGGUUC | 20 | 1418 |
| HSV1-UL19-1033 | + | CGAGCUCGUCCAGCGUCUGC | 20 | 1419 |
| HSV1-UL19-1034 | + | GCGCACGCGCGUCGUCUGGG | 20 | 1420 |
| HSV1-UL19-1035 | + | CGCGCACGCGCGUCGUCUGG | 20 | 1421 |
| HSV1-UL19-1036 | + | GCGCGCACGCGCGUCGUCUG | 20 | 1422 |
| HSV1-UL19-1037 | + | CGCGCGCACGCGCGUCGUCU | 20 | 1423 |
| HSV1-UL19-1038 | + | CCGCGCGCACGCGCGUCGUC | 20 | 1424 |
| HSV1-UL19-1039 | + | AAAGACCAGCUUCUCGCCGA | 20 | 1425 |
| HSV1-UL19-1040 | + | GGCGUAGAUGCGCUUCUCCA | 20 | 1426 |
| HSV1-UL19-1041 | + | CGGCGUAGAUGCGCUUCUCC | 20 | 1427 |
| HSV1-UL19-1042 | + | GGGGUAGGGAACGUUGGUGG | 20 | 1428 |
| HSV1-UL19-1043 | + | CAGGGGGUAGGGAACGUUGG | 20 | 1429 |
| HSV1-UL19-1044 | + | CACCAGGGGGUAGGGAACGU | 20 | 1430 |
| HSV1-UL19-1045 | + | AUGGCGCCCACCAGGGGGUA | 20 | 1431 |
| HSV1-UL19-1046 | + | CAUGGCGCCCACCAGGGGGU | 20 | 1432 |
| HSV1-UL19-1047 | + | GGUCCAUGGCGCCCACCAGG | 20 | 1433 |
| HSV1-UL19-1048 | + | AGGUCCAUGGCGCCCACCAG | 20 | 1434 |
| HSV1-UL19-1049 | + | CAGGUCCAUGGCGCCCACCA | 20 | 1435 |
| HSV1-UL19-1050 | + | UCAGGUCCAUGGCGCCCACC | 20 | 1436 |
| HSV1-UL19-1051 | + | CAGGACGAACGUCAGGUCCA | 20 | 1437 |
| HSV1-UL19-1052 | + | CCAGGGGCAGGACGAACGUC | 20 | 1438 |
| HSV1-UL19-1053 | + | GAUUGAACAGGCCCAGGGGC | 20 | 1439 |
| HSV1-UL19-1054 | + | ACCGGAUUGAACAGGCCCAG | 20 | 1440 |
| HSV1-UL19-1055 | + | GACCGGAUUGAACAGGCCCA | 20 | 1441 |
| HSV1-UL19-1056 | + | UGACCGGAUUGAACAGGCCC | 20 | 1442 |
| HSV1-UL19-1057 | + | GUUCCAUGACCGGAUUGAAC | 20 | 1443 |
| HSV1-UL19-1058 | + | GCGGCAAACCGUUCCAUGAC | 20 | 1444 |
| HSV1-UL19-1059 | + | GACUAGGUCCCCGGCGUGCG | 20 | 1445 |
| HSV1-UL19-1060 | + | GGGGGCGGGGACUAGGUCCC | 20 | 1446 |
| HSV1-UL19-1061 | + | GGUGGCCGGGGGCGGGGACU | 20 | 1447 |
| HSV1-UL19-1062 | + | GAUCCGGGUGGCCGGGGGCG | 20 | 1448 |
| HSV1-UL19-1063 | + | GGAUCCGGGUGGCCGGGGGC | 20 | 1449 |
| HSV1-UL19-1064 | + | GGGAUCCGGGUGGCCGGGGG | 20 | 1450 |
| HSV1-UL19-1065 | + | GCGGGGAUCCGGGUGGCCGG | 20 | 1451 |
| HSV1-UL19-1066 | + | CGCGGGGAUCCGGGUGGCCG | 20 | 1452 |
| HSV1-UL19-1067 | + | GCGCGGGGAUCCGGGUGGCC | 20 | 1453 |
| HSV1-UL19-1068 | + | GGCGCGGGGAUCCGGGUGGC | 20 | 1454 |
| HSV1-UL19-1069 | + | GGAAGGCGCGGGGAUCCGGG | 20 | 1455 |
| HSV1-UL19-1070 | + | GCGGGAAGGCGCGGGGAUCC | 20 | 1456 |
| HSV1-UL19-1071 | + | GGCGGGAAGGCGCGGGGAUC | 20 | 1457 |
| HSV1-UL19-1072 | + | UGGCGGGGCGGGAAGGCGCG | 20 | 1458 |
| HSV1-UL19-1073 | + | CUGGCGGGGCGGGAAGGCGC | 20 | 1459 |
| HSV1-UL19-1074 | + | GCUGGCGGGGCGGGAAGGCG | 20 | 1460 |
| HSV1-UL19-1075 | + | AAACAGCUGGCGGGGCGGGA | 20 | 1461 |
| HSV1-UL19-1076 | + | AAAAAAACAGCUGGCGGGGC | 20 | 1462 |
| HSV1-UL19-1077 | + | CAAAAAAACAGCUGGCGGGG | 20 | 1463 |
| HSV1-UL19-1078 | + | CCCCAAAAAAACAGCUGGCG | 20 | 1464 |
| HSV1-UL19-1079 | + | CCCCCAAAAAAACAGCUGGC | 20 | 1465 |
| HSV1-UL19-1080 | + | UCCCCCAAAAAAACAGCUGG | 20 | 1466 |
| HSV1-UL19-1081 | + | CCUUCCCCCAAAAAAACAGC | 20 | 1467 |
| HSV1-UL19-1082 | + | GAGACAGGCGCAGCACCUGG | 20 | 1468 |
| HSV1-UL19-1083 | + | CCAGAGACAGGCGCAGCACC | 20 | 1469 |
| HSV1-UL19-1084 | + | CGAUCGCGUGUUCCAGAGAC | 20 | 1470 |
| HSV1-UL19-1085 | + | CAUCAGCGAAGGGUGGCACA | 20 | 1471 |
| HSV1-UL19-1086 | + | CAACGUUCAUCAGCGAAGGG | 20 | 1472 |
| HSV1-UL19-1087 | + | CGUCAACGUUCAUCAGCGAA | 20 | 1473 |
| HSV1-UL19-1088 | + | GCGUCAACGUUCAUCAGCGA | 20 | 1474 |
| HSV1-UL19-1089 | + | CUUCGACGGGGUCGCGGUUA | 20 | 1475 |
| HSV1-UL19-1090 | + | UGGCGGCUUCGACGGGGUCG | 20 | 1476 |
| HSV1-UL19-1091 | + | ACGGAUUGGCGGCUUCGACG | 20 | 1477 |
| HSV1-UL19-1092 | + | UACGGAUUGGCGGCUUCGAC | 20 | 1478 |
| HSV1-UL19-1093 | + | GUACGGAUUGGCGGCUUCGA | 20 | 1479 |
| HSV1-UL19-1094 | + | GUACGCCCCGUACGGAUUGG | 20 | 1480 |
| HSV1-UL19-1095 | + | CACGUACGCCCCGUACGGAU | 20 | 1481 |
| HSV1-UL19-1096 | + | GCCGCCACGUACGCCCCGUA | 20 | 1482 |
| HSV1-UL19-1097 | + | GUCUGCGGCGGGGCCGGCCG | 20 | 1483 |
| HSV1-UL19-1098 | + | UGUCUGCGGCGGGGCCGGCC | 20 | 1484 |
| HSV1-UL19-1099 | + | AUGUCUGCGGCGGGGCCGGC | 20 | 1485 |
| HSV1-UL19-1100 | + | CUGCAUGUCUGCGGCGGGGC | 20 | 1486 |
| HSV1-UL19-1101 | + | GCUGCUGCAUGUCUGCGGCG | 20 | 1487 |
| HSV1-UL19-1102 | + | AGCUGCUGCAUGUCUGCGGC | 20 | 1488 |
| HSV1-UL19-1103 | + | CAGCUGCUGCAUGUCUGCGG | 20 | 1489 |
| HSV1-UL19-1104 | + | AAACAGCUGCUGCAUGUCUG | 20 | 1490 |
| HSV1-UL19-1105 | + | GUGGGCCAGGCGCUGCCCCC | 20 | 1491 |
| HSV1-UL19-1106 | + | AUCGGACCCGCCCGUGGGCC | 20 | 1492 |
| HSV1-UL19-1107 | + | GACCCAUCGGACCCGCCCGU | 20 | 1493 |
| HSV1-UL19-1108 | + | CGACCCAUCGGACCCGCCCG | 20 | 1494 |
| HSV1-UL19-1109 | + | UCUGGCCUUCCGCGACCCAU | 20 | 1495 |
| HSV1-UL19-1110 | + | UAAACUGCUCCGGGGUCAUC | 20 | 1496 |
| HSV1-UL19-1111 | + | GGGCUGCAUAAACUGCUCCG | 20 | 1497 |
| HSV1-UL19-1112 | + | CGGGCUGCAUAAACUGCUCC | 20 | 1498 |
| HSV1-UL19-1113 | + | UCGGGCUGCAUAAACUGCUC | 20 | 1499 |
| HSV1-UL19-1114 | + | AGAGCCAGGUUGGCGUUGUC | 20 | 1500 |
| HSV1-UL19-1115 | + | AAGAGCCAGGUUGGCGUUGU | 20 | 1501 |
| HSV1-UL19-1116 | + | GUGCAGCUCAAGAGCCAGGU | 20 | 1502 |
| HSV1-UL19-1117 | + | CGGGGUGCAGCUCAAGAGCC | 20 | 1503 |
| HSV1-UL19-1118 | + | CCACAAAGAAGUCGAACGCG | 20 | 1504 |
| HSV1-UL19-1119 | + | CCCACAAAGAAGUCGAACGC | 20 | 1505 |
| HSV1-UL19-1120 | + | CCCCACAAAGAAGUCGAACG | 20 | 1506 |
| HSV1-UL19-1121 | + | CCCCCCCGGCAGCUCCACGU | 20 | 1507 |
| HSV1-UL19-1122 | + | GCCGGGGGAACGUCCCCCCC | 20 | 1508 |
| HSV1-UL19-1123 | + | AUCUCCCCCGGGCCGGCCGG | 20 | 1509 |
| HSV1-UL19-1124 | + | GAUCUCCCCCGGGCCGGCCG | 20 | 1510 |
| HSV1-UL19-1125 | + | GGAUCUCCCCCGGGCCGGCC | 20 | 1511 |
| HSV1-UL19-1126 | + | UGGAUCUCCCCCGGGCCGGC | 20 | 1512 |
| HSV1-UL19-1127 | + | GGCCUGGAUCUCCCCCGGGC | 20 | 1513 |
| HSV1-UL19-1128 | + | AGGUGGCCUGGAUCUCCCCC | 20 | 1514 |
| HSV1-UL19-1129 | + | CAGGUGGCCUGGAUCUCCCC | 20 | 1515 |
| HSV1-UL19-1130 | + | UCACCACGCGCCAGGUGGCC | 20 | 1516 |
| HSV1-UL19-1131 | + | GCCGUUCACCACGCGCCAGG | 20 | 1517 |
| HSV1-UL19-1132 | + | GUUGCCGUUCACCACGCGCC | 20 | 1518 |
| HSV1-UL19-1133 | + | GCCGCCGGACAUAGCGCCAG | 20 | 1519 |
| HSV1-UL19-1134 | + | CGCCGCCGGACAUAGCGCCA | 20 | 1520 |
| HSV1-UL19-1135 | + | ACGCCGCCGGACAUAGCGCC | 20 | 1521 |
| HSV1-UL19-1136 | + | CGGGCGUCCCGGAACGCCGC | 20 | 1522 |
| HSV1-UL19-1137 | + | GCUCCAGGCCCCGGGCGUCC | 20 | 1523 |
| HSV1-UL19-1138 | + | CACCCCCAGCUCCAGGCCCC | 20 | 1524 |
| HSV1-UL19-1139 | + | CCACCCCCAGCUCCAGGCCC | 20 | 1525 |
| HSV1-UL19-1140 | + | GGCGUCCCACCCCCAGCUCC | 20 | 1526 |
| HSV1-UL19-1141 | + | UGGUGGCGGGGGCCAUGGCG | 20 | 1527 |
| HSV1-UL19-1142 | + | GGCGAUGGUGGCGGGGGCCA | 20 | 1528 |
| HSV1-UL19-1143 | + | AACGGCGGCGAUGGUGGCGG | 20 | 1529 |
| HSV1-UL19-1144 | + | GAACGGCGGCGAUGGUGGCG | 20 | 1530 |
| HSV1-UL19-1145 | + | CGAACGGCGGCGAUGGUGGC | 20 | 1531 |
| HSV1-UL19-1146 | + | GCGAACGGCGGCGAUGGUGG | 20 | 1532 |
| HSV1-UL19-1147 | + | CCCGCGAACGGCGGCGAUGG | 20 | 1533 |
| HSV1-UL19-1148 | + | CGCCCCGCGAACGGCGGCGA | 20 | 1534 |
| HSV1-UL19-1149 | + | GUCGAACGCCCCGCGAACGG | 20 | 1535 |
| HSV1-UL19-1150 | + | GUCGUCGAACGCCCCGCGAA | 20 | 1536 |
| HSV1-UL19-1151 | + | AAAACACCGCCGGGUAGUUG | 20 | 1537 |
| HSV1-UL19-1152 | + | GCAGCAGGUAAAACACCGCC | 20 | 1538 |
| HSV1-UL19-1153 | + | UGCAGCAGGUAAAACACCGC | 20 | 1539 |
| HSV1-UL19-1154 | + | CGUGUAUGGCGGCCUGCAGC | 20 | 1540 |
| HSV1-UL19-1155 | + | GUGCUCGCUGCCGUGUAUGG | 20 | 1541 |
| HSV1-UL19-1156 | + | GACGUGCUCGCUGCCGUGUA | 20 | 1542 |
| HSV1-UL19-1157 | + | CUGGACCACGAGCCGGGCCA | 20 | 1543 |
| HSV1-UL19-1158 | + | ACUGGACCACGAGCCGGGCC | 20 | 1544 |
| HSV1-UL19-1159 | + | GAUGCACUGGACCACGAGCC | 20 | 1545 |
| HSV1-UL19-1160 | + | UGAUGCACUGGACCACGAGC | 20 | 1546 |
| HSV1-UL19-1161 | + | UCCAGUAGCUGGUGAUGCAC | 20 | 1547 |
| HSV1-UL19-1162 | + | CCGCGUGUUGUUCCAGUAGC | 20 | 1548 |
| HSV1-UL19-1163 | + | GGGAAGGUCUCCCCCGAGGU | 20 | 1549 |
| HSV1-UL19-1164 | + | CCUCGGGAAGGUCUCCCCCG | 20 | 1550 |
| HSV1-UL19-1165 | + | CGGCCAUGCACUCCUCGGGA | 20 | 1551 |
| HSV1-UL19-1166 | + | UACACGGCCAUGCACUCCUC | 20 | 1552 |
| HSV1-UL19-1167 | + | GUACACGGCCAUGCACUCCU | 20 | 1553 |
| HSV1-UL19-1168 | + | CGCCACCAGGUCCCGGUACA | 20 | 1554 |
| HSV1-UL19-1169 | + | CGACGUGCGCCACCAGGUCC | 20 | 1555 |
| HSV1-UL19-1170 | + | GGGCCUCGACGUGCGCCACC | 20 | 1556 |
| HSV1-UL19-1171 | + | GUCAUCAACCAGCUGGGCCA | 20 | 1557 |
| HSV1-UL19-1172 | + | AGUCAUCAACCAGCUGGGCC | 20 | 1558 |
| HSV1-UL19-1173 | + | GGUAAAGUCAUCAACCAGCU | 20 | 1559 |
| HSV1-UL19-1174 | + | GGGUAAAGUCAUCAACCAGC | 20 | 1560 |
| HSV1-UL19-1175 | + | GCCCAGCUCCGGGCCGGUCA | 20 | 1561 |
| HSV1-UL19-1176 | + | CGCCCAGCUCCGGGCCGGUC | 20 | 1562 |
| HSV1-UL19-1177 | + | CUGCCCGCCCAGCUCCGGGC | 20 | 1563 |
| HSV1-UL19-1178 | + | GCGCCUGCCCGCCCAGCUCC | 20 | 1564 |
| HSV1-UL19-1179 | + | UGCGCCUGCCCGCCCAGCUC | 20 | 1565 |
| HSV1-UL19-1180 | + | UCGCAUUAGGUGAUUCAGCU | 20 | 1566 |
| HSV1-UL19-1181 | + | GCAGCGCCGGGUCUCGCAUU | 20 | 1567 |
| HSV1-UL19-1182 | + | CGAGGGGUGGCAGCAGCGCC | 20 | 1568 |
| HSV1-UL19-1183 | + | ACGAGGGGUGGCAGCAGCGC | 20 | 1569 |
| HSV1-UL19-1184 | + | UCACAGUCCCACACGAGGGG | 20 | 1570 |
| HSV1-UL19-1185 | + | GCGUCACAGUCCCACACGAG | 20 | 1571 |
| HSV1-UL19-1186 | + | GGCGUCACAGUCCCACACGA | 20 | 1572 |
| HSV1-UL19-1187 | + | GGGCGUCACAGUCCCACACG | 20 | 1573 |
| HSV1-UL19-1188 | + | CAGGGCCGCGCGCCGCAUCA | 20 | 1574 |
| HSV1-UL19-1189 | + | CCAGGGCCGCGCGCCGCAUC | 20 | 1575 |
| HSV1-UL19-1190 | + | GCAGUCGCGAUGGCGGUCCA | 20 | 1576 |
| HSV1-UL19-1191 | + | GGCAGUCGCGAUGGCGGUCC | 20 | 1577 |
| HSV1-UL19-1192 | + | UAACCCGGCAGUCGCGAUGG | 20 | 1578 |
| HSV1-UL19-1193 | + | CGCUAACCCGGCAGUCGCGA | 20 | 1579 |
| HSV1-UL19-1194 | + | CGUGGCCCCCCGCGCUAACC | 20 | 1580 |
| HSV1-UL19-1195 | + | CCGCCGCGUACACGGGGUCG | 20 | 1581 |
| HSV1-UL19-1196 | + | UACAUGCCGCCGCGUACACG | 20 | 1582 |
| HSV1-UL19-1197 | + | UUACAUGCCGCCGCGUACAC | 20 | 1583 |
| HSV1-UL19-1198 | + | GUUACAUGCCGCCGCGUACA | 20 | 1584 |
| HSV1-UL19-1199 | + | GUCGUUGCGGUUGAAGUCCG | 20 | 1585 |
| HSV1-UL19-1200 | + | GCAGCAGCUGGCCGUCGUUG | 20 | 1586 |
| HSV1-UL19-1201 | + | CCUGGGUGUUGUGCAGCAGC | 20 | 1587 |
| HSV1-UL19-1202 | + | GGCGUCCGCGGCUCGGGCCU | 20 | 1588 |
| HSV1-UL19-1203 | + | CGGCGUCCGCGGCUCGGGCC | 20 | 1589 |
| HSV1-UL19-1204 | + | AUCCGCGGCGUCCGCGGCUC | 20 | 1590 |
| HSV1-UL19-1205 | + | CAUCCGCGGCGUCCGCGGCU | 20 | 1591 |
| HSV1-UL19-1206 | + | CCGGUCAUCCGCGGCGUCCG | 20 | 1592 |
| HSV1-UL19-1207 | + | CCGGUGCGGCCGGUCAUCCG | 20 | 1593 |
| HSV1-UL19-1208 | + | AGUCCGCCCCCCGGUGCGGC | 20 | 1594 |
| HSV1-UL19-1209 | + | GUCCAGUCCGCCCCCCGGUG | 20 | 1595 |
| HSV1-UL19-1210 | + | GCACGGUCCAGUCCGCCCCC | 20 | 1596 |
| HSV1-UL19-1211 | + | GUAGUAAAUCUUGUGGUGCA | 20 | 1597 |
| HSV1-UL19-1212 | + | UCACGUAGUAGUAAAUCUUG | 20 | 1598 |
| HSV1-UL19-1213 | + | CAGCGGCCCCGCGAGAAGGC | 20 | 1599 |
| HSV1-UL19-1214 | + | GCAGCGGCCCCGCGAGAAGG | 20 | 1600 |
| HSV1-UL19-1215 | + | GCAGCAGCGGCCCCGCGAGA | 20 | 1601 |
| HSV1-UL19-1216 | + | GAACCCCCGCGGUGCAGCAG | 20 | 1602 |
| HSV1-UL19-1217 | + | GCGGUCGAAGCGAACCCCCG | 20 | 1603 |
| HSV1-UL19-1218 | + | UCUGCAGGGUGGCGUAUACG | 20 | 1604 |
| HSV1-UL19-1219 | + | GACCACCAUGUUCUGCAGGG | 20 | 1605 |
| HSV1-UL19-1220 | + | CGGGACCACCAUGUUCUGCA | 20 | 1606 |
| HSV1-UL19-1221 | + | CCGGGACCACCAUGUUCUGC | 20 | 1607 |
| HSV1-UL19-1222 | + | CCUCGCCGGGGGCGAUCUCC | 20 | 1608 |
| HSV1-UL19-1223 | + | UCCUCGCCGGGGGCGAUCUC | 20 | 1609 |
| HSV1-UL19-1224 | + | GCUGGGGCACUCCUCGCCGG | 20 | 1610 |
| HSV1-UL19-1225 | + | CGCUGGGGCACUCCUCGCCG | 20 | 1611 |
| HSV1-UL19-1226 | + | UCGCUGGGGCACUCCUCGCC | 20 | 1612 |
| HSV1-UL19-1227 | + | GUCGCUGGGGCACUCCUCGC | 20 | 1613 |
| HSV1-UL19-1228 | + | GGUCCGUCACGGGGUCGCUG | 20 | 1614 |
| HSV1-UL19-1229 | + | GGGUCCGUCACGGGGUCGCU | 20 | 1615 |
| HSV1-UL19-1230 | + | GGGGUCCGUCACGGGGUCGC | 20 | 1616 |
| HSV1-UL19-1231 | + | GGUGCGCGGGGUCCGUCACG | 20 | 1617 |
| HSV1-UL19-1232 | + | GGGUGCGCGGGGUCCGUCAC | 20 | 1618 |
| HSV1-UL19-1233 | + | GGGGUGCGCGGGGUCCGUCA | 20 | 1619 |
| HSV1-UL19-1234 | + | CCGGGUGCAGGGGGUGCGCG | 20 | 1620 |
| HSV1-UL19-1235 | + | GCCGGGUGCAGGGGGUGCGC | 20 | 1621 |
| HSV1-UL19-1236 | + | GGCCGGGUGCAGGGGGUGCG | 20 | 1622 |
| HSV1-UL19-1237 | + | CCAGAUUGGCCGGGUGCAGG | 20 | 1623 |
| HSV1-UL19-1238 | + | ACCAGAUUGGCCGGGUGCAG | 20 | 1624 |
| HSV1-UL19-1239 | + | CACCAGAUUGGCCGGGUGCA | 20 | 1625 |
| HSV1-UL19-1240 | + | CCACCAGAUUGGCCGGGUGC | 20 | 1626 |
| HSV1-UL19-1241 | + | UGUUGGCCACCAGAUUGGCC | 20 | 1627 |
| HSV1-UL19-1242 | + | GUGUUGGCCACCAGAUUGGC | 20 | 1628 |
| HSV1-UL19-1243 | + | GACCGUGUUGGCCACCAGAU | 20 | 1629 |
| HSV1-UL19-1244 | + | AAACAUGGCGUUGACCGUGU | 20 | 1630 |
| HSV1-UL19-1245 | + | CACGCGCCCGUUGUGAAACA | 20 | 1631 |
| HSV1-UL19-1246 | + | ACCUGCAGCGUGAGCAUGGC | 20 | 1632 |
| HSV1-UL19-1247 | + | CACCUGCAGCGUGAGCAUGG | 20 | 1633 |
| HSV1-UL19-1248 | + | CAGCACCUGCAGCGUGAGCA | 20 | 1634 |
| HSV1-UL19-1249 | + | UGUGCGUUCGGCCAUGUUGU | 20 | 1635 |
| HSV1-UL19-1250 | + | UUGUGCGUUCGGCCAUGUUG | 20 | 1636 |
| HSV1-UL19-1251 | + | GAGCAGCGCCGUUGUGCGUU | 20 | 1637 |
| HSV1-UL19-1252 | + | GCGGUGUUGGCGCCCGCGUC | 20 | 1638 |
| HSV1-UL19-1253 | + | CGCGGUGUUGGCGCCCGCGU | 20 | 1639 |
| HSV1-UL19-1254 | + | GUUGGUGGUGGACGCGGUGU | 20 | 1640 |
| HSV1-UL19-1255 | + | GCGCAUGUUGGUGGUGGACG | 20 | 1641 |
| HSV1-UL19-1256 | + | GAAUAUGCGCAUGUUGGUGG | 20 | 1642 |
| HSV1-UL19-1257 | + | GUCGAAUAUGCGCAUGUUGG | 20 | 1643 |
| HSV1-UL19-1258 | + | CCCGUCGAAUAUGCGCAUGU | 20 | 1644 |
| HSV1-UL19-1259 | + | GGGGGCCAUCAGCAGGAUUC | 20 | 1645 |
| HSV1-UL19-1260 | + | GAUGCUGGGGGGCCAUCAGC | 20 | 1646 |
| HSV1-UL19-1261 | + | GGUAUGGUCCAGAUGCUGGG | 20 | 1647 |
| HSV1-UL19-1262 | + | UGGUAUGGUCCAGAUGCUGG | 20 | 1648 |
| HSV1-UL19-1263 | + | AUGGUAUGGUCCAGAUGCUG | 20 | 1649 |
| HSV1-UL19-1264 | + | GAUGGUAUGGUCCAGAUGCU | 20 | 1650 |
| HSV1-UL19-1265 | + | GGAUGGUAUGGUCCAGAUGC | 20 | 1651 |
| HSV1-UL19-1266 | + | AGUCGCCAUUUUGGAUGGUA | 20 | 1652 |
| HSV1-UL19-1267 | + | AAAAUAGUCGCCAUUUUGGA | 20 | 1653 |
| HSV1-UL19-1268 | + | GGUAAAAAUAGUCGCCAUUU | 20 | 1654 |
| HSV1-UL19-1269 | + | ACAGCGCGUGGACGGGGAGG | 20 | 1655 |
| HSV1-UL19-1270 | + | AACAGCGCGUGGACGGGGAG | 20 | 1656 |
| HSV1-UL19-1271 | + | GAACAGCGCGUGGACGGGGA | 20 | 1657 |
| HSV1-UL19-1272 | + | CGAACAGCGCGUGGACGGGG | 20 | 1658 |
| HSV1-UL19-1273 | + | CGGCGAACAGCGCGUGGACG | 20 | 1659 |
| HSV1-UL19-1274 | + | CCGGCGAACAGCGCGUGGAC | 20 | 1660 |
| HSV1-UL19-1275 | + | CCCGGCGAACAGCGCGUGGA | 20 | 1661 |
| HSV1-UL19-1276 | + | CGGCCCCGGCGAACAGCGCG | 20 | 1662 |
| HSV1-UL19-1277 | + | GUUCGCCACGUGGUCGGCCC | 20 | 1663 |
| HSV1-UL19-1278 | + | GGGCGCGUUCGCCACGUGGU | 20 | 1664 |
| HSV1-UL19-1279 | + | AAUUGGGCGCGUUCGCCACG | 20 | 1665 |
| HSV1-UL19-1280 | + | CGCAGGGCCGGGGGGAAAUU | 20 | 1666 |
| HSV1-UL19-1281 | + | GCGCAGGGCCGGGGGGAAAU | 20 | 1667 |
| HSV1-UL19-1282 | + | ACAGGUCGCGCAGGGCCGGG | 20 | 1668 |
| HSV1-UL19-1283 | + | GACAGGUCGCGCAGGGCCGG | 20 | 1669 |
| HSV1-UL19-1284 | + | CGACAGGUCGCGCAGGGCCG | 20 | 1670 |
| HSV1-UL19-1285 | + | GCGACAGGUCGCGCAGGGCC | 20 | 1671 |
| HSV1-UL19-1286 | + | CGCGACAGGUCGCGCAGGGC | 20 | 1672 |
| HSV1-UL19-1287 | + | CUGCCGCGACAGGUCGCGCA | 20 | 1673 |
| HSV1-UL19-1288 | + | CCUGCCGCGACAGGUCGCGC | 20 | 1674 |
| HSV1-UL19-1289 | + | CCAGGGGGACCUGCCGCGAC | 20 | 1675 |
| HSV1-UL19-1290 | + | CCAGAGCCGGGGGGACCAGG | 20 | 1676 |
| HSV1-UL19-1291 | + | CCCAGAGCCGGGGGGACCAG | 20 | 1677 |
| HSV1-UL19-1292 | + | CCCCAGAGCCGGGGGGACCA | 20 | 1678 |
| HSV1-UL19-1293 | + | CCCCCAGAGCCGGGGGGACC | 20 | 1679 |
| HSV1-UL19-1294 | + | AGUUGGCCCCCAGAGCCGGG | 20 | 1680 |
| HSV1-UL19-1295 | + | UAGUUGGCCCCCAGAGCCGG | 20 | 1681 |
| HSV1-UL19-1296 | + | GUAGUUGGCCCCCAGAGCCG | 20 | 1682 |
| HSV1-UL19-1297 | + | AGUAGUUGGCCCCCAGAGCC | 20 | 1683 |
| HSV1-UL19-1298 | + | AAGUAGUUGGCCCCCAGAGC | 20 | 1684 |
| HSV1-UL19-1299 | + | UCGGAUCGACGAAAAGUAGU | 20 | 1685 |
| HSV1-UL19-1300 | + | CGUGCUGCACGACGGGCUGU | 20 | 1686 |
| HSV1-UL19-1301 | + | UCGCGGACGUGCUGCACGAC | 20 | 1687 |
| HSV1-UL19-1302 | + | CUCGCGGACGUGCUGCACGA | 20 | 1688 |
| HSV1-UL19-1303 | + | UCUCCCCGGCCGCGCUCUCG | 20 | 1689 |
| HSV1-UL19-1304 | + | GUAGGUCAGCGCGUUCUCCC | 20 | 1690 |
| HSV1-UL19-1305 | + | GUACCCCGCCAUGAGCGCGU | 20 | 1691 |
| HSV1-UL19-1306 | + | AGCUGAUGAUGCAAGGCCAC | 20 | 1692 |
| HSV1-UL19-1307 | + | GAGCUGAUGAUGCAAGGCCA | 20 | 1693 |
| HSV1-UL19-1308 | + | CGUCUUGAGCUGAUGAUGCA | 20 | 1694 |
| HSV1-UL19-1309 | + | UGAACCCAAACCCGGGAUGG | 20 | 1695 |
| HSV1-UL19-1310 | + | CGGUGAACCCAAACCCGGGA | 20 | 1696 |
| HSV1-UL19-1311 | + | ACGACGGUGAACCCAAACCC | 20 | 1697 |
| HSV1-UL19-1312 | + | GACGACGGUGAACCCAAACC | 20 | 1698 |
| HSV1-UL19-1313 | + | AAAGCGGUCCUGUCGGACGA | 20 | 1699 |
| HSV1-UL19-1314 | + | CAGUCACAAAGCGGUCCUGU | 20 | 1700 |
| HSV1-UL19-1315 | + | GCACGUUCUCAGUCACAAAG | 20 | 1701 |
| HSV1-UL19-1316 | + | GGGCCACCUGGAGCUGGCCC | 20 | 1702 |
| HSV1-UL19-1317 | + | CGUGCCGGGCCACCUGGAGC | 20 | 1703 |
| HSV1-UL19-1318 | + | CAGUUUCGUGCCGGGCCACC | 20 | 1704 |
| HSV1-UL19-1319 | + | CCCCCCGCCAGUUUCGUGCC | 20 | 1705 |
| HSV1-UL19-1320 | + | CCCCCCCGCCAGUUUCGUGC | 20 | 1706 |
| HSV1-UL19-1321 | + | GUCCACGUUCGCGCGCGGCU | 20 | 1707 |
| HSV1-UL19-1322 | + | GGUCCACGUUCGCGCGCGGC | 20 | 1708 |
| HSV1-UL19-1323 | + | CCCAGGUCCACGUUCGCGCG | 20 | 1709 |
| HSV1-UL19-1324 | + | CGGCGGUGUAGCCCACGCCC | 20 | 1710 |
| HSV1-UL19-1325 | + | GGUUGCCGUGGCCACGACGG | 20 | 1711 |
| HSV1-UL19-1326 | + | GACGGUUGCCGUGGCCACGA | 20 | 1712 |
| HSV1-UL19-1327 | + | GGGGUUGCGGACGGUUGCCG | 20 | 1713 |
| HSV1-UL19-1328 | + | GUCGGUGACGGGGUUGCGGA | 20 | 1714 |
| HSV1-UL19-1329 | + | CCAUGUCGGUGACGGGGUUG | 20 | 1715 |
| HSV1-UL19-1330 | + | GGUUGCCCAUGUCGGUGACG | 20 | 1716 |
| HSV1-UL19-1331 | + | AGGUUGCCCAUGUCGGUGAC | 20 | 1717 |
| HSV1-UL19-1332 | + | AAGGUUGCCCAUGUCGGUGA | 20 | 1718 |
| HSV1-UL19-1333 | + | UUGGGGAAGGUUGCCCAUGU | 20 | 1719 |
| HSV1-UL19-1334 | + | CCAGGUAAAAGUUUUGGGGA | 20 | 1720 |
| HSV1-UL19-1335 | + | CGGCCCAGGUAAAAGUUUUG | 20 | 1721 |
| HSV1-UL19-1336 | + | GCGGCCCAGGUAAAAGUUUU | 20 | 1722 |
| HSV1-UL19-1337 | + | CGCGGCCCAGGUAAAAGUUU | 20 | 1723 |
| HSV1-UL19-1338 | + | GAGGGGGAGCCCCGCGGCCC | 20 | 1724 |
| HSV1-UL19-1339 | + | CCAGGAGAGGGGGAGCCCCG | 20 | 1725 |
| HSV1-UL19-1340 | + | GCCGCGUUGUCCAGGAGAGG | 20 | 1726 |
| HSV1-UL19-1341 | + | UGCCGCGUUGUCCAGGAGAG | 20 | 1727 |
| HSV1-UL19-1342 | + | CUGCCGCGUUGUCCAGGAGA | 20 | 1728 |
| HSV1-UL19-1343 | + | GCUGCCGCGUUGUCCAGGAG | 20 | 1729 |
| HSV1-UL19-1344 | + | ACACGGCUGCCGCGUUGUCC | 20 | 1730 |
| HSV1-UL19-1345 | + | GACCGCGUUCCGCAGGUACA | 20 | 1731 |
| HSV1-UL19-1346 | + | CCGCCACGACCGCGUUCCGC | 20 | 1732 |
| HSV1-UL19-1347 | + | CGGGCUGGGCCGGCCCCAGG | 20 | 1733 |
| HSV1-UL19-1348 | + | GGACGGGCUGGGCCGGCCCC | 20 | 1734 |
| HSV1-UL19-1349 | + | AACACGGGGACGGGCUGGGC | 20 | 1735 |
| HSV1-UL19-1350 | + | CCCGAACACGGGGACGGGCU | 20 | 1736 |
| HSV1-UL19-1351 | + | ACCCGAACACGGGGACGGGC | 20 | 1737 |
| HSV1-UL19-1352 | + | GCGCACCCGAACACGGGGAC | 20 | 1738 |
| HSV1-UL19-1353 | + | GGCGCACCCGAACACGGGGA | 20 | 1739 |
| HSV1-UL19-1354 | + | CCUGGGCGCACCCGAACACG | 20 | 1740 |
| HSV1-UL19-1355 | + | ACCUGGGCGCACCCGAACAC | 20 | 1741 |
| HSV1-UL19-1356 | + | CACCUGGGCGCACCCGAACA | 20 | 1742 |
| HSV1-UL19-1357 | + | CCCUGCGCGCCGCGGCACCU | 20 | 1743 |
| HSV1-UL19-1358 | + | UCCCUGCGCGCCGCGGCACC | 20 | 1744 |
| HSV1-UL19-1359 | + | UGGUCCAUCCCUGCGCGCCG | 20 | 1745 |
| HSV1-UL19-1360 | + | CACACACGGCGUCCUGGCCG | 20 | 1746 |
| HSV1-UL19-1361 | + | UAAACUCACACACGGCGUCC | 20 | 1747 |
| HSV1-UL19-1362 | + | GGUGGCGAUAAACUCACACA | 20 | 1748 |
| HSV1-UL19-1363 | + | GACGUCGGUCGACACGGGGG | 20 | 1749 |
| HSV1-UL19-1364 | + | GUUGACGUCGGUCGACACGG | 20 | 1750 |
| HSV1-UL19-1365 | + | AGUUGACGUCGGUCGACACG | 20 | 1751 |
| HSV1-UL19-1366 | + | UAGUUGACGUCGGUCGACAC | 20 | 1752 |
| HSV1-UL19-1367 | + | GUAGUUGACGUCGGUCGACA | 20 | 1753 |
| HSV1-UL19-1368 | + | CCGGCGGAAGUAGUUGACGU | 20 | 1754 |
| HSV1-UL19-1369 | + | CCCGGGGGUUGCAGGGCCGG | 20 | 1755 |
| HSV1-UL19-1370 | + | GUCCCCGGGGGUUGCAGGGC | 20 | 1756 |
| HSV1-UL19-1371 | + | GCGCGUCCCCGGGGGUUGCA | 20 | 1757 |
| HSV1-UL19-1372 | + | GGCGCGUCCCCGGGGGUUGC | 20 | 1758 |
| HSV1-UL19-1373 | + | CCCCGGCGGCGCGUCCCCGG | 20 | 1759 |
| HSV1-UL19-1374 | + | CCCCCGGCGGCGCGUCCCCG | 20 | 1760 |
| HSV1-UL19-1375 | + | GCCCCCGGCGGCGCGUCCCC | 20 | 1761 |
| HSV1-UL19-1376 | + | CGCCCCCGGCGGCGCGUCCC | 20 | 1762 |
| HSV1-UL19-1377 | + | CCCCGCGUAAACGCCCCCGG | 20 | 1763 |
| HSV1-UL19-1378 | + | GUCCCCCGCGUAAACGCCCC | 20 | 1764 |
| HSV1-UL19-1379 | + | GCCGUGGUCGUACAUGAGGG | 20 | 1765 |
| HSV1-UL19-1380 | + | CUGGCCGUGGUCGUACAUGA | 20 | 1766 |
| HSV1-UL19-1381 | + | UCUGGCCGUGGUCGUACAUG | 20 | 1767 |
| HSV1-UL19-1382 | + | GGGACGGGUCGCUCUGGCCG | 20 | 1768 |
| HSV1-UL19-1383 | + | AGGCCCGGGACGGGUCGCUC | 20 | 1769 |
| HSV1-UL19-1384 | + | UGGCCGCGAAGGCCCGGGAC | 20 | 1770 |
| HSV1-UL19-1385 | + | GUGGCCGCGAAGGCCCGGGA | 20 | 1771 |
| HSV1-UL19-1386 | + | GGCCGUGGCCGCGAAGGCCC | 20 | 1772 |
| HSV1-UL19-1387 | + | UGGCCGUGGCCGCGAAGGCC | 20 | 1773 |
| HSV1-UL19-1388 | + | CGGGUUGGCCGUGGCCGCGA | 20 | 1774 |
| HSV1-UL19-1389 | + | CGACGCCCACGGGUUGGCCG | 20 | 1775 |
| HSV1-UL19-1390 | + | UCGCUGCGACGCCCACGGGU | 20 | 1776 |
| HSV1-UL19-1391 | + | AAAAUCGCUGCGACGCCCAC | 20 | 1777 |
| HSV1-UL19-1392 | + | GAAAAUCGCUGCGACGCCCA | 20 | 1778 |
| HSV1-UL19-1393 | + | GGUAGGCCCCGUUAUAGAGC | 20 | 1779 |
| HSV1-UL19-1394 | + | CGAGGCCCCGUUGAGGUGGU | 20 | 1780 |
| HSV1-UL19-1395 | + | CCGGCGAGGCCCCGUUGAGG | 20 | 1781 |
| HSV1-UL19-1396 | + | GCACCGGCGAGGCCCCGUUG | 20 | 1782 |
| HSV1-UL19-1397 | + | GCAGGGGCUGAGCACCGGCG | 20 | 1783 |
| HSV1-UL19-1398 | + | UUAAAGCAGGGGCUGAGCAC | 20 | 1784 |
| HSV1-UL19-1399 | + | ACGUAAAGAACUUAAAGCAG | 20 | 1785 |
| HSV1-UL19-1400 | + | GACGUAAAGAACUUAAAGCA | 20 | 1786 |
| HSV1-UL19-1401 | + | CGACGUAAAGAACUUAAAGC | 20 | 1787 |
| HSV1-UL19-1402 | + | GCGAUGUUUGGCGGCGAUGU | 20 | 1788 |
| HSV1-UL19-1403 | + | CUCCAGGCAGCGAUGUUUGG | 20 | 1789 |
| HSV1-UL19-1404 | + | GCGCUCCAGGCAGCGAUGUU | 20 | 1790 |
| HSV1-UL19-1405 | + | UCUCCACGAUCAGGCGCUCC | 20 | 1791 |
| HSV1-UL19-1406 | + | CCGAACCCGUCUCCACGAUC | 20 | 1792 |
| HSV1-UL19-1407 | + | GUCGCUGGCGGCGGUGGCCG | 20 | 1793 |
| HSV1-UL19-1408 | + | CUGUACGUCGCUGGCGGCGG | 20 | 1794 |
| HSV1-UL19-1409 | + | AAACUGUACGUCGCUGGCGG | 20 | 1795 |
| HSV1-UL19-1410 | + | CUUAAACUGUACGUCGCUGG | 20 | 1796 |
| HSV1-UL19-1411 | + | GCGCUUAAACUGUACGUCGC | 20 | 1797 |
| HSV1-UL19-1412 | + | CGAGUUCGCGGCACCCCGGG | 20 | 1798 |
| HSV1-UL19-1413 | + | ACGAGUUCGCGGCACCCCGG | 20 | 1799 |
| HSV1-UL19-1414 | + | CACGAGUUCGCGGCACCCCG | 20 | 1800 |
| HSV1-UL19-1415 | + | CCACGAGUUCGCGGCACCCC | 20 | 1801 |
| HSV1-UL19-1416 | + | UCCACGAGUUCGCGGCACCC | 20 | 1802 |
| HSV1-UL19-1417 | + | ACGGGUCCUCCACGAGUUCG | 20 | 1803 |
| HSV1-UL19-1418 | + | CCUCCUGAAACAGGCCACAC | 20 | 1804 |
| HSV1-UL19-1419 | + | GCCUCCUGAAACAGGCCACA | 20 | 1805 |
| HSV1-UL19-1420 | + | GCGGGUAGGCCUCCUGAAAC | 20 | 1806 |
| HSV1-UL19-1421 | + | GCUGGCGCAGGUGAGCGGGU | 20 | 1807 |
| HSV1-UL19-1422 | + | GGUCGCUGGCGCAGGUGAGC | 20 | 1808 |
| HSV1-UL19-1423 | + | GGGUCGCUGGCGCAGGUGAG | 20 | 1809 |
| HSV1-UL19-1424 | + | GAGGGCGGGGUCGCUGGCGC | 20 | 1810 |
| HSV1-UL19-1425 | + | GCGGAGGAGGGCGGGGUCGC | 20 | 1811 |
| HSV1-UL19-1426 | + | GGGCACUGCGGAGGAGGGCG | 20 | 1812 |
| HSV1-UL19-1427 | + | CGGGCACUGCGGAGGAGGGC | 20 | 1813 |
| HSV1-UL19-1428 | + | GCGGGCACUGCGGAGGAGGG | 20 | 1814 |
| HSV1-UL19-1429 | + | GUUGCGGGCACUGCGGAGGA | 20 | 1815 |
| HSV1-UL19-1430 | + | CGUUGCGGGCACUGCGGAGG | 20 | 1816 |
| HSV1-UL19-1431 | + | CCCCGUUGCGGGCACUGCGG | 20 | 1817 |
| HSV1-UL19-1432 | + | CUUCCCCGUUGCGGGCACUG | 20 | 1818 |
| HSV1-UL19-1433 | + | CGCGUGGGCUUCCCCGUUGC | 20 | 1819 |
| HSV1-UL19-1434 | + | GCGCGUGGGCUUCCCCGUUG | 20 | 1820 |
| HSV1-UL19-1435 | + | GAAGUGGGUCUCCCGCGCGU | 20 | 1821 |
| HSV1-UL19-1436 | + | CGAAGUGGGUCUCCCGCGCG | 20 | 1822 |
| HSV1-UL19-1437 | + | GACGAGAUACUGCGCGAAGU | 20 | 1823 |
| HSV1-UL19-1438 | + | AGACGAGAUACUGCGCGAAG | 20 | 1824 |
| HSV1-UL19-1439 | + | CAGAGCCAGUCCCUUGAGCG | 20 | 1825 |
| HSV1-UL19-1440 | + | ACAGAGCCAGUCCCUUGAGC | 20 | 1826 |
| HSV1-UL19-1441 | + | UACAGAGCCAGUCCCUUGAG | 20 | 1827 |
| HSV1-UL19-1442 | + | ACGCCCCGAGGACGGCC | 17 | 1828 |
| HSV1-UL19-1443 | + | CUCAAACGCCCCGAGGA | 17 | 1829 |
| HSV1-UL19-1444 | + | CGCGCUCAAACGCCCCG | 17 | 1830 |
| HSV1-UL19-1445 | + | CACGUGCAGCAUCUGGU | 17 | 1831 |
| HSV1-UL19-1446 | + | GCAGCACGUGCAGCAUC | 17 | 1832 |
| HSV1-UL19-1447 | + | AACAACAAAGCCAGGGG | 17 | 1833 |
| HSV1-UL19-1448 | + | GGCAACAACAAAGCCAG | 17 | 1834 |
| HSV1-UL19-1449 | + | CGGCAACAACAAAGCCA | 17 | 1835 |
| HSV1-UL19-1450 | + | UCGGCAACAACAAAGCC | 17 | 1836 |
| HSV1-UL19-1451 | + | UCCAGGUAUCGUUGCAU | 17 | 1837 |
| HSV1-UL19-1452 | + | CCAGGCGGCCGUUGUCC | 17 | 1838 |
| HSV1-UL19-1453 | + | CCACCCUGGUGGCCAGG | 17 | 1839 |
| HSV1-UL19-1454 | + | GGGCCACCCUGGUGGCC | 17 | 1840 |
| HSV1-UL19-1455 | + | CGCCCGGGCCACCCUGG | 17 | 1841 |
| HSV1-UL19-1456 | + | GGUCGCCCGGGCCACCC | 17 | 1842 |
| HSV1-UL19-1457 | + | GGCGACCAGGGUCGCCC | 17 | 1843 |
| HSV1-UL19-1458 | + | CGGCGACCAGGGUCGCC | 17 | 1844 |
| HSV1-UL19-1459 | + | CUUUAGCUCGGCGACCA | 17 | 1845 |
| HSV1-UL19-1460 | + | GCUUUAGCUCGGCGACC | 17 | 1846 |
| HSV1-UL19-1461 | + | GAAGCUUCGCUUUAGCU | 17 | 1847 |
| HSV1-UL19-1462 | + | GGUGGCCCGCCUUGCCC | 17 | 1848 |
| HSV1-UL19-1463 | + | CGACGGCCUCCCGGCGG | 17 | 1849 |
| HSV1-UL19-1464 | + | CCUCGACGGCCUCCCGG | 17 | 1850 |
| HSV1-UL19-1465 | + | AGGCCUCGACGGCCUCC | 17 | 1851 |
| HSV1-UL19-1466 | + | CACGAGCCAGGCCUCGA | 17 | 1852 |
| HSV1-UL19-1467 | + | GGUGAGGUCCACGAGCC | 17 | 1853 |
| HSV1-UL19-1468 | + | GCUGCGUGGCCGUGGUG | 17 | 1854 |
| HSV1-UL19-1469 | + | GGAGGGCUGCGUGGCCG | 17 | 1855 |
| HSV1-UL19-1470 | + | GGCCACGGAGGGCUGCG | 17 | 1856 |
| HSV1-UL19-1471 | + | CGGGGCACGGCCACGGA | 17 | 1857 |
| HSV1-UL19-1472 | + | ACGGGGCACGGCCACGG | 17 | 1858 |
| HSV1-UL19-1473 | + | CAGACGGGGCACGGCCA | 17 | 1859 |
| HSV1-UL19-1474 | + | AUGCGUCAGACGGGGCA | 17 | 1860 |
| HSV1-UL19-1475 | + | UCGGCAUGCGUCAGACG | 17 | 1861 |
| HSV1-UL19-1476 | + | GUCGGCAUGCGUCAGAC | 17 | 1862 |
| HSV1-UL19-1477 | + | UGUCGGCAUGCGUCAGA | 17 | 1863 |
| HSV1-UL19-1478 | + | CGGCCGCCCGCGCGUGU | 17 | 1864 |
| HSV1-UL19-1479 | + | ACGAGCACCCCGUCGAC | 17 | 1865 |
| HSV1-UL19-1480 | + | CUGUUUGAUCGGGGCGG | 17 | 1866 |
| HSV1-UL19-1481 | + | GCGCUGUUUGAUCGGGG | 17 | 1867 |
| HSV1-UL19-1482 | + | GAGGCGCUGUUUGAUCG | 17 | 1868 |
| HSV1-UL19-1483 | + | GGAGGCGCUGUUUGAUC | 17 | 1869 |
| HSV1-UL19-1484 | + | AGGAGGCGCUGUUUGAU | 17 | 1870 |
| HSV1-UL19-1485 | + | UCAGGAAGGACUGCAGG | 17 | 1871 |
| HSV1-UL19-1486 | + | CUUUCAGGAAGGACUGC | 17 | 1872 |
| HSV1-UL19-1487 | + | GUCCUCCACUUUCAGGA | 17 | 1873 |
| HSV1-UL19-1488 | + | CGGUGUCCUCCACUUUC | 17 | 1874 |
| HSV1-UL19-1489 | + | CACCGGCACGUCGGCUU | 17 | 1875 |
| HSV1-UL19-1490 | + | GUACGUCACCGGCACGU | 17 | 1876 |
| HSV1-UL19-1491 | + | AUCUCGCCGUACGUCAC | 17 | 1877 |
| HSV1-UL19-1492 | + | CCAGGUUGGCCCCGUUC | 17 | 1878 |
| HSV1-UL19-1493 | + | GAGCGCCGUGACCAGGU | 17 | 1879 |
| HSV1-UL19-1494 | + | UCACGAGCGCCGUGACC | 17 | 1880 |
| HSV1-UL19-1495 | + | GUCGUCCAGACUUCGCA | 17 | 1881 |
| HSV1-UL19-1496 | + | GCAUCUCCAGCAGGUGG | 17 | 1882 |
| HSV1-UL19-1497 | + | CCUGCAUCUCCAGCAGG | 17 | 1883 |
| HSV1-UL19-1498 | + | CCUCCUGCAUCUCCAGC | 17 | 1884 |
| HSV1-UL19-1499 | + | CCAGCGUCUGCCGGUUC | 17 | 1885 |
| HSV1-UL19-1500 | + | GCUCGUCCAGCGUCUGC | 17 | 1886 |
| HSV1-UL19-1501 | + | CACGCGCGUCGUCUGGG | 17 | 1887 |
| HSV1-UL19-1502 | + | GCACGCGCGUCGUCUGG | 17 | 1888 |
| HSV1-UL19-1503 | + | CGCACGCGCGUCGUCUG | 17 | 1889 |
| HSV1-UL19-1504 | + | GCGCACGCGCGUCGUCU | 17 | 1890 |
| HSV1-UL19-1505 | + | CGCGCACGCGCGUCGUC | 17 | 1891 |
| HSV1-UL19-1506 | + | GACCAGCUUCUCGCCGA | 17 | 1892 |
| HSV1-UL19-1507 | + | GUAGAUGCGCUUCUCCA | 17 | 1893 |
| HSV1-UL19-1508 | + | CGUAGAUGCGCUUCUCC | 17 | 1894 |
| HSV1-UL19-1509 | + | GUAGGGAACGUUGGUGG | 17 | 1895 |
| HSV1-UL19-1510 | + | GGGGUAGGGAACGUUGG | 17 | 1896 |
| HSV1-UL19-1511 | + | CAGGGGGUAGGGAACGU | 17 | 1897 |
| HSV1-UL19-1512 | + | GCGCCCACCAGGGGGUA | 17 | 1898 |
| HSV1-UL19-1513 | + | GGCGCCCACCAGGGGGU | 17 | 1899 |
| HSV1-UL19-1514 | + | CCAUGGCGCCCACCAGG | 17 | 1900 |
| HSV1-UL19-1515 | + | UCCAUGGCGCCCACCAG | 17 | 1901 |
| HSV1-UL19-1516 | + | GUCCAUGGCGCCCACCA | 17 | 1902 |
| HSV1-UL19-1517 | + | GGUCCAUGGCGCCCACC | 17 | 1903 |
| HSV1-UL19-1518 | + | GACGAACGUCAGGUCCA | 17 | 1904 |
| HSV1-UL19-1519 | + | GGGGCAGGACGAACGUC | 17 | 1905 |
| HSV1-UL19-1520 | + | UGAACAGGCCCAGGGGC | 17 | 1906 |
| HSV1-UL19-1521 | + | GGAUUGAACAGGCCCAG | 17 | 1907 |
| HSV1-UL19-1522 | + | CGGAUUGAACAGGCCCA | 17 | 1908 |
| HSV1-UL19-1523 | + | CCGGAUUGAACAGGCCC | 17 | 1909 |
| HSV1-UL19-1524 | + | CCAUGACCGGAUUGAAC | 17 | 1910 |
| HSV1-UL19-1525 | + | GCAAACCGUUCCAUGAC | 17 | 1911 |
| HSV1-UL19-1526 | + | UAGGUCCCCGGCGUGCG | 17 | 1912 |
| HSV1-UL19-1527 | + | GGCGGGGACUAGGUCCC | 17 | 1913 |
| HSV1-UL19-1528 | + | GGCCGGGGGCGGGGACU | 17 | 1914 |
| HSV1-UL19-1529 | + | CCGGGUGGCCGGGGGCG | 17 | 1915 |
| HSV1-UL19-1530 | + | UCCGGGUGGCCGGGGGC | 17 | 1916 |
| HSV1-UL19-1531 | + | AUCCGGGUGGCCGGGGG | 17 | 1917 |
| HSV1-UL19-1532 | + | GGGAUCCGGGUGGCCGG | 17 | 1918 |
| HSV1-UL19-1533 | + | GGGGAUCCGGGUGGCCG | 17 | 1919 |
| HSV1-UL19-1534 | + | CGGGGAUCCGGGUGGCC | 17 | 1920 |
| HSV1-UL19-1535 | + | GCGGGGAUCCGGGUGGC | 17 | 1921 |
| HSV1-UL19-1536 | + | AGGCGCGGGGAUCCGGG | 17 | 1922 |
| HSV1-UL19-1537 | + | GGAAGGCGCGGGGAUCC | 17 | 1923 |
| HSV1-UL19-1538 | + | GGGAAGGCGCGGGGAUC | 17 | 1924 |
| HSV1-UL19-1539 | + | CGGGGCGGGAAGGCGCG | 17 | 1925 |
| HSV1-UL19-1540 | + | GCGGGGCGGGAAGGCGC | 17 | 1926 |
| HSV1-UL19-1541 | + | GGCGGGGCGGGAAGGCG | 17 | 1927 |
| HSV1-UL19-1542 | + | CAGCUGGCGGGGCGGGA | 17 | 1928 |
| HSV1-UL19-1543 | + | AAAACAGCUGGCGGGGC | 17 | 1929 |
| HSV1-UL19-1544 | + | AAAAACAGCUGGCGGGG | 17 | 1930 |
| HSV1-UL19-1545 | + | CAAAAAAACAGCUGGCG | 17 | 1931 |
| HSV1-UL19-1546 | + | CCAAAAAAACAGCUGGC | 17 | 1932 |
| HSV1-UL19-1547 | + | CCCAAAAAAACAGCUGG | 17 | 1933 |
| HSV1-UL19-1548 | + | UCCCCCAAAAAAACAGC | 17 | 1934 |
| HSV1-UL19-1549 | + | ACAGGCGCAGCACCUGG | 17 | 1935 |
| HSV1-UL19-1550 | + | GAGACAGGCGCAGCACC | 17 | 1936 |
| HSV1-UL19-1551 | + | UCGCGUGUUCCAGAGAC | 17 | 1937 |
| HSV1-UL19-1552 | + | CAGCGAAGGGUGGCACA | 17 | 1938 |
| HSV1-UL19-1553 | + | CGUUCAUCAGCGAAGGG | 17 | 1939 |
| HSV1-UL19-1554 | + | CAACGUUCAUCAGCGAA | 17 | 1940 |
| HSV1-UL19-1555 | + | UCAACGUUCAUCAGCGA | 17 | 1941 |
| HSV1-UL19-1556 | + | CGACGGGGUCGCGGUUA | 17 | 1942 |
| HSV1-UL19-1557 | + | CGGCUUCGACGGGGUCG | 17 | 1943 |
| HSV1-UL19-1558 | + | GAUUGGCGGCUUCGACG | 17 | 1944 |
| HSV1-UL19-1559 | + | GGAUUGGCGGCUUCGAC | 17 | 1945 |
| HSV1-UL19-1560 | + | CGGAUUGGCGGCUUCGA | 17 | 1946 |
| HSV1-UL19-1561 | + | CGCCCCGUACGGAUUGG | 17 | 1947 |
| HSV1-UL19-1562 | + | GUACGCCCCGUACGGAU | 17 | 1948 |
| HSV1-UL19-1563 | + | GCCACGUACGCCCCGUA | 17 | 1949 |
| HSV1-UL19-1564 | + | UGCGGCGGGGCCGGCCG | 17 | 1950 |
| HSV1-UL19-1565 | + | CUGCGGCGGGGCCGGCC | 17 | 1951 |
| HSV1-UL19-1566 | + | UCUGCGGCGGGGCCGGC | 17 | 1952 |
| HSV1-UL19-1567 | + | CAUGUCUGCGGCGGGGC | 17 | 1953 |
| HSV1-UL19-1568 | + | GCUGCAUGUCUGCGGCG | 17 | 1954 |
| HSV1-UL19-1569 | + | UGCUGCAUGUCUGCGGC | 17 | 1955 |
| HSV1-UL19-1570 | + | CUGCUGCAUGUCUGCGG | 17 | 1956 |
| HSV1-UL19-1571 | + | CAGCUGCUGCAUGUCUG | 17 | 1957 |
| HSV1-UL19-1572 | + | GGCCAGGCGCUGCCCCC | 17 | 1958 |
| HSV1-UL19-1573 | + | GGACCCGCCCGUGGGCC | 17 | 1959 |
| HSV1-UL19-1574 | + | CCAUCGGACCCGCCCGU | 17 | 1960 |
| HSV1-UL19-1575 | + | CCCAUCGGACCCGCCCG | 17 | 1961 |
| HSV1-UL19-1576 | + | GGCCUUCCGCGACCCAU | 17 | 1962 |
| HSV1-UL19-1577 | + | ACUGCUCCGGGGUCAUC | 17 | 1963 |
| HSV1-UL19-1578 | + | CUGCAUAAACUGCUCCG | 17 | 1964 |
| HSV1-UL19-1579 | + | GCUGCAUAAACUGCUCC | 17 | 1965 |
| HSV1-UL19-1580 | + | GGCUGCAUAAACUGCUC | 17 | 1966 |
| HSV1-UL19-1581 | + | GCCAGGUUGGCGUUGUC | 17 | 1967 |
| HSV1-UL19-1582 | + | AGCCAGGUUGGCGUUGU | 17 | 1968 |
| HSV1-UL19-1583 | + | CAGCUCAAGAGCCAGGU | 17 | 1969 |
| HSV1-UL19-1584 | + | GGUGCAGCUCAAGAGCC | 17 | 1970 |
| HSV1-UL19-1585 | + | CAAAGAAGUCGAACGCG | 17 | 1971 |
| HSV1-UL19-1586 | + | ACAAAGAAGUCGAACGC | 17 | 1972 |
| HSV1-UL19-1587 | + | CACAAAGAAGUCGAACG | 17 | 1973 |
| HSV1-UL19-1588 | + | CCCCGGCAGCUCCACGU | 17 | 1974 |
| HSV1-UL19-1589 | + | GGGGGAACGUCCCCCCC | 17 | 1975 |
| HSV1-UL19-1590 | + | UCCCCCGGGCCGGCCGG | 17 | 1976 |
| HSV1-UL19-1591 | + | CUCCCCCGGGCCGGCCG | 17 | 1977 |
| HSV1-UL19-1592 | + | UCUCCCCCGGGCCGGCC | 17 | 1978 |
| HSV1-UL19-1593 | + | AUCUCCCCCGGGCCGGC | 17 | 1979 |
| HSV1-UL19-1594 | + | CUGGAUCUCCCCCGGGC | 17 | 1980 |
| HSV1-UL19-1595 | + | UGGCCUGGAUCUCCCCC | 17 | 1981 |
| HSV1-UL19-1596 | + | GUGGCCUGGAUCUCCCC | 17 | 1982 |
| HSV1-UL19-1597 | + | CCACGCGCCAGGUGGCC | 17 | 1983 |
| HSV1-UL19-1598 | + | GUUCACCACGCGCCAGG | 17 | 1984 |
| HSV1-UL19-1599 | + | GCCGUUCACCACGCGCC | 17 | 1985 |
| HSV1-UL19-1600 | + | GCCGGACAUAGCGCCAG | 17 | 1986 |
| HSV1-UL19-1601 | + | CGCCGGACAUAGCGCCA | 17 | 1987 |
| HSV1-UL19-1602 | + | CCGCCGGACAUAGCGCC | 17 | 1988 |
| HSV1-UL19-1603 | + | GCGUCCCGGAACGCCGC | 17 | 1989 |
| HSV1-UL19-1604 | + | CCAGGCCCCGGGCGUCC | 17 | 1990 |
| HSV1-UL19-1605 | + | CCCCAGCUCCAGGCCCC | 17 | 1991 |
| HSV1-UL19-1606 | + | CCCCCAGCUCCAGGCCC | 17 | 1992 |
| HSV1-UL19-1607 | + | GUCCCACCCCCAGCUCC | 17 | 1993 |
| HSV1-UL19-1608 | + | UGGCGGGGGCCAUGGCG | 17 | 1994 |
| HSV1-UL19-1609 | + | GAUGGUGGCGGGGGCCA | 17 | 1995 |
| HSV1-UL19-1610 | + | GGCGGCGAUGGUGGCGG | 17 | 1996 |
| HSV1-UL19-1611 | + | CGGCGGCGAUGGUGGCG | 17 | 1997 |
| HSV1-UL19-1612 | + | ACGGCGGCGAUGGUGGC | 17 | 1998 |
| HSV1-UL19-1613 | + | AACGGCGGCGAUGGUGG | 17 | 1999 |
| HSV1-UL19-1614 | + | GCGAACGGCGGCGAUGG | 17 | 2000 |
| HSV1-UL19-1615 | + | CCCGCGAACGGCGGCGA | 17 | 2001 |
| HSV1-UL19-1616 | + | GAACGCCCCGCGAACGG | 17 | 2002 |
| HSV1-UL19-1617 | + | GUCGAACGCCCCGCGAA | 17 | 2003 |
| HSV1-UL19-1618 | + | ACACCGCCGGGUAGUUG | 17 | 2004 |
| HSV1-UL19-1619 | + | GCAGGUAAAACACCGCC | 17 | 2005 |
| HSV1-UL19-1620 | + | AGCAGGUAAAACACCGC | 17 | 2006 |
| HSV1-UL19-1621 | + | GUAUGGCGGCCUGCAGC | 17 | 2007 |
| HSV1-UL19-1622 | + | CUCGCUGCCGUGUAUGG | 17 | 2008 |
| HSV1-UL19-1623 | + | GUGCUCGCUGCCGUGUA | 17 | 2009 |
| HSV1-UL19-1624 | + | GACCACGAGCCGGGCCA | 17 | 2010 |
| HSV1-UL19-1625 | + | GGACCACGAGCCGGGCC | 17 | 2011 |
| HSV1-UL19-1626 | + | GCACUGGACCACGAGCC | 17 | 2012 |
| HSV1-UL19-1627 | + | UGCACUGGACCACGAGC | 17 | 2013 |
| HSV1-UL19-1628 | + | AGUAGCUGGUGAUGCAC | 17 | 2014 |
| HSV1-UL19-1629 | + | CGUGUUGUUCCAGUAGC | 17 | 2015 |
| HSV1-UL19-1630 | + | AAGGUCUCCCCCGAGGU | 17 | 2016 |
| HSV1-UL19-1631 | + | CGGGAAGGUCUCCCCCG | 17 | 2017 |
| HSV1-UL19-1632 | + | CCAUGCACUCCUCGGGA | 17 | 2018 |
| HSV1-UL19-1633 | + | ACGGCCAUGCACUCCUC | 17 | 2019 |
| HSV1-UL19-1634 | + | CACGGCCAUGCACUCCU | 17 | 2020 |
| HSV1-UL19-1635 | + | CACCAGGUCCCGGUACA | 17 | 2021 |
| HSV1-UL19-1636 | + | CGUGCGCCACCAGGUCC | 17 | 2022 |
| HSV1-UL19-1637 | + | CCUCGACGUGCGCCACC | 17 | 2023 |
| HSV1-UL19-1638 | + | AUCAACCAGCUGGGCCA | 17 | 2024 |
| HSV1-UL19-1639 | + | CAUCAACCAGCUGGGCC | 17 | 2025 |
| HSV1-UL19-1640 | + | AAAGUCAUCAACCAGCU | 17 | 2026 |
| HSV1-UL19-1641 | + | UAAAGUCAUCAACCAGC | 17 | 2027 |
| HSV1-UL19-1642 | + | CAGCUCCGGGCCGGUCA | 17 | 2028 |
| HSV1-UL19-1643 | + | CCAGCUCCGGGCCGGUC | 17 | 2029 |
| HSV1-UL19-1644 | + | CCCGCCCAGCUCCGGGC | 17 | 2030 |
| HSV1-UL19-1645 | + | CCUGCCCGCCCAGCUCC | 17 | 2031 |
| HSV1-UL19-1646 | + | GCCUGCCCGCCCAGCUC | 17 | 2032 |
| HSV1-UL19-1647 | + | CAUUAGGUGAUUCAGCU | 17 | 2033 |
| HSV1-UL19-1648 | + | GCGCCGGGUCUCGCAUU | 17 | 2034 |
| HSV1-UL19-1649 | + | GGGGUGGCAGCAGCGCC | 17 | 2035 |
| HSV1-UL19-1650 | + | AGGGGUGGCAGCAGCGC | 17 | 2036 |
| HSV1-UL19-1651 | + | CAGUCCCACACGAGGGG | 17 | 2037 |
| HSV1-UL19-1652 | + | UCACAGUCCCACACGAG | 17 | 2038 |
| HSV1-UL19-1653 | + | GUCACAGUCCCACACGA | 17 | 2039 |
| HSV1-UL19-1654 | + | CGUCACAGUCCCACACG | 17 | 2040 |
| HSV1-UL19-1655 | + | GGCCGCGCGCCGCAUCA | 17 | 2041 |
| HSV1-UL19-1656 | + | GGGCCGCGCGCCGCAUC | 17 | 2042 |
| HSV1-UL19-1657 | + | GUCGCGAUGGCGGUCCA | 17 | 2043 |
| HSV1-UL19-1658 | + | AGUCGCGAUGGCGGUCC | 17 | 2044 |
| HSV1-UL19-1659 | + | CCCGGCAGUCGCGAUGG | 17 | 2045 |
| HSV1-UL19-1660 | + | UAACCCGGCAGUCGCGA | 17 | 2046 |
| HSV1-UL19-1661 | + | GGCCCCCCGCGCUAACC | 17 | 2047 |
| HSV1-UL19-1662 | + | CCGCGUACACGGGGUCG | 17 | 2048 |
| HSV1-UL19-1663 | + | AUGCCGCCGCGUACACG | 17 | 2049 |
| HSV1-UL19-1664 | + | CAUGCCGCCGCGUACAC | 17 | 2050 |
| HSV1-UL19-1665 | + | ACAUGCCGCCGCGUACA | 17 | 2051 |
| HSV1-UL19-1666 | + | GUUGCGGUUGAAGUCCG | 17 | 2052 |
| HSV1-UL19-1667 | + | GCAGCUGGCCGUCGUUG | 17 | 2053 |
| HSV1-UL19-1668 | + | GGGUGUUGUGCAGCAGC | 17 | 2054 |
| HSV1-UL19-1669 | + | GUCCGCGGCUCGGGCCU | 17 | 2055 |
| HSV1-UL19-1670 | + | CGUCCGCGGCUCGGGCC | 17 | 2056 |
| HSV1-UL19-1671 | + | CGCGGCGUCCGCGGCUC | 17 | 2057 |
| HSV1-UL19-1672 | + | CCGCGGCGUCCGCGGCU | 17 | 2058 |
| HSV1-UL19-1673 | + | GUCAUCCGCGGCGUCCG | 17 | 2059 |
| HSV1-UL19-1674 | + | GUGCGGCCGGUCAUCCG | 17 | 2060 |
| HSV1-UL19-1675 | + | CCGCCCCCCGGUGCGGC | 17 | 2061 |
| HSV1-UL19-1676 | + | CAGUCCGCCCCCCGGUG | 17 | 2062 |
| HSV1-UL19-1677 | + | CGGUCCAGUCCGCCCCC | 17 | 2063 |
| HSV1-UL19-1678 | + | GUAAAUCUUGUGGUGCA | 17 | 2064 |
| HSV1-UL19-1679 | + | CGUAGUAGUAAAUCUUG | 17 | 2065 |
| HSV1-UL19-1680 | + | CGGCCCCGCGAGAAGGC | 17 | 2066 |
| HSV1-UL19-1681 | + | GCGGCCCCGCGAGAAGG | 17 | 2067 |
| HSV1-UL19-1682 | + | GCAGCGGCCCCGCGAGA | 17 | 2068 |
| HSV1-UL19-1683 | + | CCCCCGCGGUGCAGCAG | 17 | 2069 |
| HSV1-UL19-1684 | + | GUCGAAGCGAACCCCCG | 17 | 2070 |
| HSV1-UL19-1685 | + | GCAGGGUGGCGUAUACG | 17 | 2071 |
| HSV1-UL19-1686 | + | CACCAUGUUCUGCAGGG | 17 | 2072 |
| HSV1-UL19-1687 | + | GACCACCAUGUUCUGCA | 17 | 2073 |
| HSV1-UL19-1688 | + | GGACCACCAUGUUCUGC | 17 | 2074 |
| HSV1-UL19-1689 | + | CGCCGGGGGCGAUCUCC | 17 | 2075 |
| HSV1-UL19-1690 | + | UCGCCGGGGGCGAUCUC | 17 | 2076 |
| HSV1-UL19-1691 | + | GGGGCACUCCUCGCCGG | 17 | 2077 |
| HSV1-UL19-1692 | + | UGGGGCACUCCUCGCCG | 17 | 2078 |
| HSV1-UL19-1693 | + | CUGGGGCACUCCUCGCC | 17 | 2079 |
| HSV1-UL19-1694 | + | GCUGGGGCACUCCUCGC | 17 | 2080 |
| HSV1-UL19-1695 | + | CCGUCACGGGGUCGCUG | 17 | 2081 |
| HSV1-UL19-1696 | + | UCCGUCACGGGGUCGCU | 17 | 2082 |
| HSV1-UL19-1697 | + | GUCCGUCACGGGGUCGC | 17 | 2083 |
| HSV1-UL19-1698 | + | GCGCGGGGUCCGUCACG | 17 | 2084 |
| HSV1-UL19-1699 | + | UGCGCGGGGUCCGUCAC | 17 | 2085 |
| HSV1-UL19-1700 | + | GUGCGCGGGGUCCGUCA | 17 | 2086 |
| HSV1-UL19-1701 | + | GGUGCAGGGGGUGCGCG | 17 | 2087 |
| HSV1-UL19-1702 | + | GGGUGCAGGGGGUGCGC | 17 | 2088 |
| HSV1-UL19-1703 | + | CGGGUGCAGGGGGUGCG | 17 | 2089 |
| HSV1-UL19-1704 | + | GAUUGGCCGGGUGCAGG | 17 | 2090 |
| HSV1-UL19-1705 | + | AGAUUGGCCGGGUGCAG | 17 | 2091 |
| HSV1-UL19-1706 | + | CAGAUUGGCCGGGUGCA | 17 | 2092 |
| HSV1-UL19-1707 | + | CCAGAUUGGCCGGGUGC | 17 | 2093 |
| HSV1-UL19-1708 | + | UGGCCACCAGAUUGGCC | 17 | 2094 |
| HSV1-UL19-1709 | + | UUGGCCACCAGAUUGGC | 17 | 2095 |
| HSV1-UL19-1710 | + | CGUGUUGGCCACCAGAU | 17 | 2096 |
| HSV1-UL19-1711 | + | CAUGGCGUUGACCGUGU | 17 | 2097 |
| HSV1-UL19-1712 | + | GCGCCCGUUGUGAAACA | 17 | 2098 |
| HSV1-UL19-1713 | + | UGCAGCGUGAGCAUGGC | 17 | 2099 |
| HSV1-UL19-1714 | + | CUGCAGCGUGAGCAUGG | 17 | 2100 |
| HSV1-UL19-1715 | + | CACCUGCAGCGUGAGCA | 17 | 2101 |
| HSV1-UL19-1716 | + | GCGUUCGGCCAUGUUGU | 17 | 2102 |
| HSV1-UL19-1717 | + | UGCGUUCGGCCAUGUUG | 17 | 2103 |
| HSV1-UL19-1718 | + | CAGCGCCGUUGUGCGUU | 17 | 2104 |
| HSV1-UL19-1719 | + | GUGUUGGCGCCCGCGUC | 17 | 2105 |
| HSV1-UL19-1720 | + | GGUGUUGGCGCCCGCGU | 17 | 2106 |
| HSV1-UL19-1721 | + | GGUGGUGGACGCGGUGU | 17 | 2107 |
| HSV1-UL19-1722 | + | CAUGUUGGUGGUGGACG | 17 | 2108 |
| HSV1-UL19-1723 | + | UAUGCGCAUGUUGGUGG | 17 | 2109 |
| HSV1-UL19-1724 | + | GAAUAUGCGCAUGUUGG | 17 | 2110 |
| HSV1-UL19-1725 | + | GUCGAAUAUGCGCAUGU | 17 | 2111 |
| HSV1-UL19-1726 | + | GGCCAUCAGCAGGAUUC | 17 | 2112 |
| HSV1-UL19-1727 | + | GCUGGGGGGCCAUCAGC | 17 | 2113 |
| HSV1-UL19-1728 | + | AUGGUCCAGAUGCUGGG | 17 | 2114 |
| HSV1-UL19-1729 | + | UAUGGUCCAGAUGCUGG | 17 | 2115 |
| HSV1-UL19-1730 | + | GUAUGGUCCAGAUGCUG | 17 | 2116 |
| HSV1-UL19-1731 | + | GGUAUGGUCCAGAUGCU | 17 | 2117 |
| HSV1-UL19-1732 | + | UGGUAUGGUCCAGAUGC | 17 | 2118 |
| HSV1-UL19-1733 | + | CGCCAUUUUGGAUGGUA | 17 | 2119 |
| HSV1-UL19-1734 | + | AUAGUCGCCAUUUUGGA | 17 | 2120 |
| HSV1-UL19-1735 | + | AAAAAUAGUCGCCAUUU | 17 | 2121 |
| HSV1-UL19-1736 | + | GCGCGUGGACGGGGAGG | 17 | 2122 |
| HSV1-UL19-1737 | + | AGCGCGUGGACGGGGAG | 17 | 2123 |
| HSV1-UL19-1738 | + | CAGCGCGUGGACGGGGA | 17 | 2124 |
| HSV1-UL19-1739 | + | ACAGCGCGUGGACGGGG | 17 | 2125 |
| HSV1-UL19-1740 | + | CGAACAGCGCGUGGACG | 17 | 2126 |
| HSV1-UL19-1741 | + | GCGAACAGCGCGUGGAC | 17 | 2127 |
| HSV1-UL19-1742 | + | GGCGAACAGCGCGUGGA | 17 | 2128 |
| HSV1-UL19-1743 | + | CCCCGGCGAACAGCGCG | 17 | 2129 |
| HSV1-UL19-1744 | + | CGCCACGUGGUCGGCCC | 17 | 2130 |
| HSV1-UL19-1745 | + | CGCGUUCGCCACGUGGU | 17 | 2131 |
| HSV1-UL19-1746 | + | UGGGCGCGUUCGCCACG | 17 | 2132 |
| HSV1-UL19-1747 | + | AGGGCCGGGGGGAAAUU | 17 | 2133 |
| HSV1-UL19-1748 | + | CAGGGCCGGGGGGAAAU | 17 | 2134 |
| HSV1-UL19-1749 | + | GGUCGCGCAGGGCCGGG | 17 | 2135 |
| HSV1-UL19-1750 | + | AGGUCGCGCAGGGCCGG | 17 | 2136 |
| HSV1-UL19-1751 | + | CAGGUCGCGCAGGGCCG | 17 | 2137 |
| HSV1-UL19-1752 | + | ACAGGUCGCGCAGGGCC | 17 | 2138 |
| HSV1-UL19-1753 | + | GACAGGUCGCGCAGGGC | 17 | 2139 |
| HSV1-UL19-1754 | + | CCGCGACAGGUCGCGCA | 17 | 2140 |
| HSV1-UL19-1755 | + | GCCGCGACAGGUCGCGC | 17 | 2141 |
| HSV1-UL19-1756 | + | GGGGGACCUGCCGCGAC | 17 | 2142 |
| HSV1-UL19-1757 | + | GAGCCGGGGGGACCAGG | 17 | 2143 |
| HSV1-UL19-1758 | + | AGAGCCGGGGGGACCAG | 17 | 2144 |
| HSV1-UL19-1759 | + | CAGAGCCGGGGGGACCA | 17 | 2145 |
| HSV1-UL19-1760 | + | CCAGAGCCGGGGGGACC | 17 | 2146 |
| HSV1-UL19-1761 | + | UGGCCCCCAGAGCCGGG | 17 | 2147 |
| HSV1-UL19-1762 | + | UUGGCCCCCAGAGCCGG | 17 | 2148 |
| HSV1-UL19-1763 | + | GUUGGCCCCCAGAGCCG | 17 | 2149 |
| HSV1-UL19-1764 | + | AGUUGGCCCCCAGAGCC | 17 | 2150 |
| HSV1-UL19-1765 | + | UAGUUGGCCCCCAGAGC | 17 | 2151 |
| HSV1-UL19-1766 | + | GAUCGACGAAAAGUAGU | 17 | 2152 |
| HSV1-UL19-1767 | + | GCUGCACGACGGGCUGU | 17 | 2153 |
| HSV1-UL19-1768 | + | CGGACGUGCUGCACGAC | 17 | 2154 |
| HSV1-UL19-1769 | + | GCGGACGUGCUGCACGA | 17 | 2155 |
| HSV1-UL19-1770 | + | CCCCGGCCGCGCUCUCG | 17 | 2156 |
| HSV1-UL19-1771 | + | GGUCAGCGCGUUCUCCC | 17 | 2157 |
| HSV1-UL19-1772 | + | CCCCGCCAUGAGCGCGU | 17 | 2158 |
| HSV1-UL19-1773 | + | UGAUGAUGCAAGGCCAC | 17 | 2159 |
| HSV1-UL19-1774 | + | CUGAUGAUGCAAGGCCA | 17 | 2160 |
| HSV1-UL19-1775 | + | CUUGAGCUGAUGAUGCA | 17 | 2161 |
| HSV1-UL19-1776 | + | ACCCAAACCCGGGAUGG | 17 | 2162 |
| HSV1-UL19-1777 | + | UGAACCCAAACCCGGGA | 17 | 2163 |
| HSV1-UL19-1778 | + | ACGGUGAACCCAAACCC | 17 | 2164 |
| HSV1-UL19-1779 | + | GACGGUGAACCCAAACC | 17 | 2165 |
| HSV1-UL19-1780 | + | GCGGUCCUGUCGGACGA | 17 | 2166 |
| HSV1-UL19-1781 | + | UCACAAAGCGGUCCUGU | 17 | 2167 |
| HSV1-UL19-1782 | + | CGUUCUCAGUCACAAAG | 17 | 2168 |
| HSV1-UL19-1783 | + | CCACCUGGAGCUGGCCC | 17 | 2169 |
| HSV1-UL19-1784 | + | GCCGGGCCACCUGGAGC | 17 | 2170 |
| HSV1-UL19-1785 | + | UUUCGUGCCGGGCCACC | 17 | 2171 |
| HSV1-UL19-1786 | + | CCCGCCAGUUUCGUGCC | 17 | 2172 |
| HSV1-UL19-1787 | + | CCCCGCCAGUUUCGUGC | 17 | 2173 |
| HSV1-UL19-1788 | + | CACGUUCGCGCGCGGCU | 17 | 2174 |
| HSV1-UL19-1789 | + | CCACGUUCGCGCGCGGC | 17 | 2175 |
| HSV1-UL19-1790 | + | AGGUCCACGUUCGCGCG | 17 | 2176 |
| HSV1-UL19-1791 | + | CGGUGUAGCCCACGCCC | 17 | 2177 |
| HSV1-UL19-1792 | + | UGCCGUGGCCACGACGG | 17 | 2178 |
| HSV1-UL19-1793 | + | GGUUGCCGUGGCCACGA | 17 | 2179 |
| HSV1-UL19-1794 | + | GUUGCGGACGGUUGCCG | 17 | 2180 |
| HSV1-UL19-1795 | + | GGUGACGGGGUUGCGGA | 17 | 2181 |
| HSV1-UL19-1796 | + | UGUCGGUGACGGGGUUG | 17 | 2182 |
| HSV1-UL19-1797 | + | UGCCCAUGUCGGUGACG | 17 | 2183 |
| HSV1-UL19-1798 | + | UUGCCCAUGUCGGUGAC | 17 | 2184 |
| HSV1-UL19-1799 | + | GUUGCCCAUGUCGGUGA | 17 | 2185 |
| HSV1-UL19-1800 | + | GGGAAGGUUGCCCAUGU | 17 | 2186 |
| HSV1-UL19-1801 | + | GGUAAAAGUUUUGGGGA | 17 | 2187 |
| HSV1-UL19-1802 | + | CCCAGGUAAAAGUUUUG | 17 | 2188 |
| HSV1-UL19-1803 | + | GCCCAGGUAAAAGUUUU | 17 | 2189 |
| HSV1-UL19-1804 | + | GGCCCAGGUAAAAGUUU | 17 | 2190 |
| HSV1-UL19-1805 | + | GGGGAGCCCCGCGGCCC | 17 | 2191 |
| HSV1-UL19-1806 | + | GGAGAGGGGGAGCCCCG | 17 | 2192 |
| HSV1-UL19-1807 | + | GCGUUGUCCAGGAGAGG | 17 | 2193 |
| HSV1-UL19-1808 | + | CGCGUUGUCCAGGAGAG | 17 | 2194 |
| HSV1-UL19-1809 | + | CCGCGUUGUCCAGGAGA | 17 | 2195 |
| HSV1-UL19-1810 | + | GCCGCGUUGUCCAGGAG | 17 | 2196 |
| HSV1-UL19-1811 | + | CGGCUGCCGCGUUGUCC | 17 | 2197 |
| HSV1-UL19-1812 | + | CGCGUUCCGCAGGUACA | 17 | 2198 |
| HSV1-UL19-1813 | + | CCACGACCGCGUUCCGC | 17 | 2199 |
| HSV1-UL19-1814 | + | GCUGGGCCGGCCCCAGG | 17 | 2200 |
| HSV1-UL19-1815 | + | CGGGCUGGGCCGGCCCC | 17 | 2201 |
| HSV1-UL19-1816 | + | ACGGGGACGGGCUGGGC | 17 | 2202 |
| HSV1-UL19-1817 | + | GAACACGGGGACGGGCU | 17 | 2203 |
| HSV1-UL19-1818 | + | CGAACACGGGGACGGGC | 17 | 2204 |
| HSV1-UL19-1819 | + | CACCCGAACACGGGGAC | 17 | 2205 |
| HSV1-UL19-1820 | + | GCACCCGAACACGGGGA | 17 | 2206 |
| HSV1-UL19-1821 | + | GGGCGCACCCGAACACG | 17 | 2207 |
| HSV1-UL19-1822 | + | UGGGCGCACCCGAACAC | 17 | 2208 |
| HSV1-UL19-1823 | + | CUGGGCGCACCCGAACA | 17 | 2209 |
| HSV1-UL19-1824 | + | UGCGCGCCGCGGCACCU | 17 | 2210 |
| HSV1-UL19-1825 | + | CUGCGCGCCGCGGCACC | 17 | 2211 |
| HSV1-UL19-1826 | + | UCCAUCCCUGCGCGCCG | 17 | 2212 |
| HSV1-UL19-1827 | + | ACACGGCGUCCUGGCCG | 17 | 2213 |
| HSV1-UL19-1828 | + | ACUCACACACGGCGUCC | 17 | 2214 |
| HSV1-UL19-1829 | + | GGCGAUAAACUCACACA | 17 | 2215 |
| HSV1-UL19-1830 | + | GUCGGUCGACACGGGGG | 17 | 2216 |
| HSV1-UL19-1831 | + | GACGUCGGUCGACACGG | 17 | 2217 |
| HSV1-UL19-1832 | + | UGACGUCGGUCGACACG | 17 | 2218 |
| HSV1-UL19-1833 | + | UUGACGUCGGUCGACAC | 17 | 2219 |
| HSV1-UL19-1834 | + | GUUGACGUCGGUCGACA | 17 | 2220 |
| HSV1-UL19-1835 | + | GCGGAAGUAGUUGACGU | 17 | 2221 |
| HSV1-UL19-1836 | + | GGGGGUUGCAGGGCCGG | 17 | 2222 |
| HSV1-UL19-1837 | + | CCCGGGGGUUGCAGGGC | 17 | 2223 |
| HSV1-UL19-1838 | + | CGUCCCCGGGGGUUGCA | 17 | 2224 |
| HSV1-UL19-1839 | + | GCGUCCCCGGGGGUUGC | 17 | 2225 |
| HSV1-UL19-1840 | + | CGGCGGCGCGUCCCCGG | 17 | 2226 |
| HSV1-UL19-1841 | + | CCGGCGGCGCGUCCCCG | 17 | 2227 |
| HSV1-UL19-1842 | + | CCCGGCGGCGCGUCCCC | 17 | 2228 |
| HSV1-UL19-1843 | + | CCCCGGCGGCGCGUCCC | 17 | 2229 |
| HSV1-UL19-1844 | + | CGCGUAAACGCCCCCGG | 17 | 2230 |
| HSV1-UL19-1845 | + | CCCCGCGUAAACGCCCC | 17 | 2231 |
| HSV1-UL19-1846 | + | GUGGUCGUACAUGAGGG | 17 | 2232 |
| HSV1-UL19-1847 | + | GCCGUGGUCGUACAUGA | 17 | 2233 |
| HSV1-UL19-1848 | + | GGCCGUGGUCGUACAUG | 17 | 2234 |
| HSV1-UL19-1849 | + | ACGGGUCGCUCUGGCCG | 17 | 2235 |
| HSV1-UL19-1850 | + | CCCGGGACGGGUCGCUC | 17 | 2236 |
| HSV1-UL19-1851 | + | CCGCGAAGGCCCGGGAC | 17 | 2237 |
| HSV1-UL19-1852 | + | GCCGCGAAGGCCCGGGA | 17 | 2238 |
| HSV1-UL19-1853 | + | CGUGGCCGCGAAGGCCC | 17 | 2239 |
| HSV1-UL19-1854 | + | CCGUGGCCGCGAAGGCC | 17 | 2240 |
| HSV1-UL19-1855 | + | GUUGGCCGUGGCCGCGA | 17 | 2241 |
| HSV1-UL19-1856 | + | CGCCCACGGGUUGGCCG | 17 | 2242 |
| HSV1-UL19-1857 | + | CUGCGACGCCCACGGGU | 17 | 2243 |
| HSV1-UL19-1858 | + | AUCGCUGCGACGCCCAC | 17 | 2244 |
| HSV1-UL19-1859 | + | AAUCGCUGCGACGCCCA | 17 | 2245 |
| HSV1-UL19-1860 | + | AGGCCCCGUUAUAGAGC | 17 | 2246 |
| HSV1-UL19-1861 | + | GGCCCCGUUGAGGUGGU | 17 | 2247 |
| HSV1-UL19-1862 | + | GCGAGGCCCCGUUGAGG | 17 | 2248 |
| HSV1-UL19-1863 | + | CCGGCGAGGCCCCGUUG | 17 | 2249 |
| HSV1-UL19-1864 | + | GGGGCUGAGCACCGGCG | 17 | 2250 |
| HSV1-UL19-1865 | + | AAGCAGGGGCUGAGCAC | 17 | 2251 |
| HSV1-UL19-1866 | + | UAAAGAACUUAAAGCAG | 17 | 2252 |
| HSV1-UL19-1867 | + | GUAAAGAACUUAAAGCA | 17 | 2253 |
| HSV1-UL19-1868 | + | CGUAAAGAACUUAAAGC | 17 | 2254 |
| HSV1-UL19-1869 | + | AUGUUUGGCGGCGAUGU | 17 | 2255 |
| HSV1-UL19-1870 | + | CAGGCAGCGAUGUUUGG | 17 | 2256 |
| HSV1-UL19-1871 | + | CUCCAGGCAGCGAUGUU | 17 | 2257 |
| HSV1-UL19-1872 | + | CCACGAUCAGGCGCUCC | 17 | 2258 |
| HSV1-UL19-1873 | + | AACCCGUCUCCACGAUC | 17 | 2259 |
| HSV1-UL19-1874 | + | GCUGGCGGCGGUGGCCG | 17 | 2260 |
| HSV1-UL19-1875 | + | UACGUCGCUGGCGGCGG | 17 | 2261 |
| HSV1-UL19-1876 | + | CUGUACGUCGCUGGCGG | 17 | 2262 |
| HSV1-UL19-1877 | + | AAACUGUACGUCGCUGG | 17 | 2263 |
| HSV1-UL19-1878 | + | CUUAAACUGUACGUCGC | 17 | 2264 |
| HSV1-UL19-1879 | + | GUUCGCGGCACCCCGGG | 17 | 2265 |
| HSV1-UL19-1880 | + | AGUUCGCGGCACCCCGG | 17 | 2266 |
| HSV1-UL19-1881 | + | GAGUUCGCGGCACCCCG | 17 | 2267 |
| HSV1-UL19-1882 | + | CGAGUUCGCGGCACCCC | 17 | 2268 |
| HSV1-UL19-1883 | + | ACGAGUUCGCGGCACCC | 17 | 2269 |
| HSV1-UL19-1884 | + | GGUCCUCCACGAGUUCG | 17 | 2270 |
| HSV1-UL19-1885 | + | CCUGAAACAGGCCACAC | 17 | 2271 |
| HSV1-UL19-1886 | + | UCCUGAAACAGGCCACA | 17 | 2272 |
| HSV1-UL19-1887 | + | GGUAGGCCUCCUGAAAC | 17 | 2273 |
| HSV1-UL19-1888 | + | GGCGCAGGUGAGCGGGU | 17 | 2274 |
| HSV1-UL19-1889 | + | CGCUGGCGCAGGUGAGC | 17 | 2275 |
| HSV1-UL19-1890 | + | UCGCUGGCGCAGGUGAG | 17 | 2276 |
| HSV1-UL19-1891 | + | GGCGGGGUCGCUGGCGC | 17 | 2277 |
| HSV1-UL19-1892 | + | GAGGAGGGCGGGGUCGC | 17 | 2278 |
| HSV1-UL19-1893 | + | CACUGCGGAGGAGGGCG | 17 | 2279 |
| HSV1-UL19-1894 | + | GCACUGCGGAGGAGGGC | 17 | 2280 |
| HSV1-UL19-1895 | + | GGCACUGCGGAGGAGGG | 17 | 2281 |
| HSV1-UL19-1896 | + | GCGGGCACUGCGGAGGA | 17 | 2282 |
| HSV1-UL19-1897 | + | UGCGGGCACUGCGGAGG | 17 | 2283 |
| HSV1-UL19-1898 | + | CGUUGCGGGCACUGCGG | 17 | 2284 |
| HSV1-UL19-1899 | + | CCCCGUUGCGGGCACUG | 17 | 2285 |
| HSV1-UL19-1900 | + | GUGGGCUUCCCCGUUGC | 17 | 2286 |
| HSV1-UL19-1901 | + | CGUGGGCUUCCCCGUUG | 17 | 2287 |
| HSV1-UL19-1902 | + | GUGGGUCUCCCGCGCGU | 17 | 2288 |
| HSV1-UL19-1903 | + | AGUGGGUCUCCCGCGCG | 17 | 2289 |
| HSV1-UL19-1904 | + | GAGAUACUGCGCGAAGU | 17 | 2290 |
| HSV1-UL19-1905 | + | CGAGAUACUGCGCGAAG | 17 | 2291 |
| HSV1-UL19-1906 | + | AGCCAGUCCCUUGAGCG | 17 | 2292 |
| HSV1-UL19-1907 | + | GAGCCAGUCCCUUGAGC | 17 | 2293 |
| HSV1-UL19-1908 | + | AGAGCCAGUCCCUUGAG | 17 | 2294 |

Table 1D provides exemplary targeting domains for knocking out the UL19 gene selected according to the first tier parameters. The targeting domains are selected based on location within first 500 bp of the coding sequence of the UL19 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 1D
1st Tier

| | | | Target | |
|---|---|---|---|---|
| | DNA | | Site | |
| gRNA Name | Strand | Targeting Domain | Length | Seq ID |
| HSV1-UL19-1909 | − | CCGCUCCCAACCGCGAC | 20 | 2295 |
| | | CCU | | |
| HSV1-UL19-1 | − | CGCUCCCAACCGCGACC | 20 | 461 |
| | | CUC | | |
| HSV1-UL19-1911 | − | UGCCGCGGCCAUGGUGC | 20 | 2296 |
| | | CGA | | |
| HSV1-UL19-1912 | − | CGGGUCCCUCCUUAGCA | 20 | 2297 |
| | | CGA | | |
| HSV1-UL19-1913 | − | UGCAAACAGCCUGUACG | 20 | 2298 |
| | | ACG | | |
| HSV1-UL19-1914 | − | ACGUCGAGUUCGACGCC | 20 | 2299 |
| | | CUG | | |
| HSV1-UL19-10 | − | CGUCGAGUUCGACGCCC | 20 | 467 |
| | | UGC | | |
| HSV1-UL19-1916 | − | CCCUGUCGCUCGUGCGC | 20 | 2300 |
| | | UUU | | |
| HSV1-UL19-13 | − | CCUGUCGCUCGUGCGCU | 20 | 395 |
| | | UUC | | |
| HSV1-UL19-1918 | − | GCUCGUGCGCUUUCUGG | 20 | 2301 |
| | | AGC | | |
| HSV1-UL19-1919 | − | CGUGCGUGUGUACCAAG | 20 | 2302 |
| | | UUU | | |
| HSV1-UL19-18 | − | GUGCGUGUGUACCAAGU | 20 | 404 |
| | | UUC | | |
| HSV1-UL19-1921 | − | AGUUUCCGGAGCUGGCC | 20 | 2303 |
| | | UAC | | |
| HSV1-UL19-1922 | − | UCCGGAGCUGGCCUACA | 20 | 2304 |
| | | UGA | | |
| HSV1-UL19-1923 | − | CGGAGCUGGCCUACAUG | 20 | 2305 |
| | | AAC | | |
| HSV1-UL19-20 | − | GGAGCUGGCCUACAUGA | 20 | 469 |
| | | ACG | | |
| HSV1-UL19-1925 | − | GAACGAGGGGCGCGUAC | 20 | 2306 |
| | | AGU | | |
| HSV1-UL19-1926 | − | CCGCGACGGCCCGCACC | 20 | 2307 |
| | | CCA | | |
| HSV1-UL19-1927 | − | AUGACCAAGAUUAUCGA | 20 | 2308 |
| | | CCG | | |
| HSV1-UL19-1928 | − | AGAUUAUCGACCGCCGG | 20 | 2309 |
| | | GCC | | |
| HSV1-UL19-1929 | − | CGCCGCCUUCAGCCUGG | 20 | 2310 |
| | | CCA | | |
| HSV1-UL19-1930 | − | CCGAGGCCAUCGCCCUG | 20 | 2311 |
| | | CUC | | |
| HSV1-UL19-29 | − | CGAGGCCAUCGCCCUGC | 20 | 474 |
| | | UCA | | |
| HSV1-UL19-30 | − | GAGGCCAUCGCCCUGCU | 20 | 475 |
| | | CAC | | |
| HSV1-UL19-31 | − | AGGCCAUCGCCCUGCUC | 20 | 417 |
| | | ACG | | |
| HSV1-UL19-32 | − | GGCCAUCGCCCUGCUCA | 20 | 476 |
| | | CGG | | |
| HSV1-UL19-1935 | − | CCCUGCUCACGGGGGAG | 20 | 2312 |
| | | GCC | | |
| HSV1-UL19-1936 | − | GCUCACGGGGGAGGCCC | 20 | 2313 |
| | | UGG | | |
| HSV1-UL19-35 | − | CUCACGGGGGAGGCCCU | 20 | 478 |
| | | GGA | | |
| HSV1-UL19-1938 | − | CGGGGGAGGCCCUGGAC | 20 | 2314 |
| | | GGG | | |
| HSV1-UL19-1939 | − | CUCCCAACCGCGACCCU | 17 | 2315 |
| HSV1-UL19-95 | − | UCCCAACCGCGACCCUC | 17 | 518 |
| HSV1-UL19-1941 | − | CGCGGCCAUGGUGCCGA | 17 | 2316 |
| HSV1-UL19-1942 | − | GUCCCUCCUUAGCACGA | 17 | 2317 |
| HSV1-UL19-1943 | − | AAACAGCCUGUACGACG | 17 | 2318 |
| HSV1-UL19-1944 | − | UCGAGUUCGACGCCCUG | 17 | 2319 |
| HSV1-UL19-104 | − | CGAGUUCGACGCCCUGC | 17 | 524 |
| HSV1-UL19-1946 | − | UGUCGCUCGUGCGCUUU | 17 | 2320 |
| HSV1-UL19-107 | − | GUCGCUCGUGCGCUUUC | 17 | 400 |
| HSV1-UL19-1948 | − | CGUGCGCUUUCUGGAGC | 17 | 2321 |
| HSV1-UL19-1949 | − | GCGUGUGUACCAAGUUU | 17 | 2322 |
| HSV1-UL19-112 | − | CGUGUGUACCAAGUUUC | 17 | 407 |
| HSV1-UL19-1951 | − | UUCCGGAGCUGGCCUAC | 17 | 2323 |
| HSV1-UL19-1952 | − | GGAGCUGGCCUACAUGA | 17 | 2324 |
| HSV1-UL19-1953 | − | AGCUGGCCUACAUGAAC | 17 | 2325 |
| HSV1-UL19-114 | − | GCUGGCCUACAUGAACG | 17 | 526 |
| HSV1-UL19-1955 | − | CGAGGGGCGCGUACAGU | 17 | 2326 |
| HSV1-UL19-1956 | − | CGACGGCCCGCACCCCA | 17 | 2327 |
| HSV1-UL19-1957 | − | ACCAAGAUUAUCGACCG | 17 | 2328 |
| HSV1-UL19-1958 | − | UUAUCGACCGCCGGGCC | 17 | 2329 |
| HSV1-UL19-1959 | − | CGCCUUCAGCCUGGCCA | 17 | 2330 |
| HSV1-UL19-1960 | − | AGGCCAUCGCCCUGCUC | 17 | 2331 |
| HSV1-UL19-123 | − | GGCCAUCGCCCUGCUCA | 17 | 531 |
| HSV1-UL19-124 | − | GCCAUCGCCCUGCUCAC | 17 | 532 |
| HSV1-UL19-125 | − | CCAUCGCCCUGCUCACG | 17 | 418 |
| HSV1-UL19-126 | − | CAUCGCCCUGCUCACGG | 17 | 533 |
| HSV1-UL19-1965 | − | UGCUCACGGGGGAGGCC | 17 | 2332 |
| HSV1-UL19-1966 | − | CACGGGGGAGGCCCUGG | 17 | 2333 |
| HSV1-UL19-129 | − | ACGGGGGAGGCCCUGGA | 17 | 535 |
| HSV1-UL19-1968 | − | GGGAGGCCCUGGACGGG | 17 | 2334 |
| HSV1-UL19-93 | + | UAUCCCGGAGGGUCGCG | 20 | 457 |
| | | GUU | | |
| HSV1-UL19-92 | + | GUAUCCCGGAGGGUCGC | 20 | 456 |
| | | GGU | | |
| HSV1-UL19-1971 | + | GGUAUCCCGGAGGGUCG | 20 | 2335 |
| | | CGG | | |
| HSV1-UL19-1972 | + | CGCGGCAUACCGGUAUC | 20 | 2336 |
| | | CCG | | |
| HSV1-UL19-88 | + | GCCGCGGCAUACCGGUA | 20 | 514 |
| | | UCC | | |
| HSV1-UL19-1974 | + | GGCCGCGGCAUACCGGU | 20 | 2337 |
| | | AUC | | |
| HSV1-UL19-81 | + | CCACCUCGAUCGUGCUA | 20 | 509 |
| | | AGG | | |
| HSV1-UL19-1976 | + | GCCACCUCGAUCGUGCU | 20 | 2338 |
| | | AAG | | |
| HSV1-UL19-80 | + | ACGCCACCUCGAUCGUG | 20 | 508 |
| | | CUA | | |
| HSV1-UL19-1978 | + | GACGCCACCUCGAUCGU | 20 | 2339 |
| | | GCU | | |
| HSV1-UL19-78 | + | GUUUGCAUCGGAGCGCA | 20 | 506 |
| | | CGC | | |
| HSV1-UL19-77 | + | UGUUUGCAUCGGAGCGC | 20 | 505 |
| | | ACG | | |
| HSV1-UL19-1981 | + | CUGUUUGCAUCGGAGCG | 20 | 2340 |
| | | CAC | | |
| HSV1-UL19-76 | + | GUCGUACAGGCUGUUUG | 20 | 442 |
| | | CAU | | |
| HSV1-UL19-1983 | + | CGUCGUACAGGCUGUUU | 20 | 2341 |
| | | GCA | | |
| HSV1-UL19-1984 | + | AAUACGACCCCAGCAGG | 20 | 2342 |
| | | GCG | | |
| HSV1-UL19-1985 | + | GUGUUGCAAUACGACCC | 20 | 2343 |
| | | CAG | | |
| HSV1-UL19-1986 | + | UCCAGAAAGCGCACGAG | 20 | 2344 |
| | | CGA | | |
| HSV1-UL19-1987 | + | ACCCGAGCUCCAGAAAG | 20 | 2345 |
| | | CGC | | |
| HSV1-UL19-1988 | + | GCCACCGACAACCCGAG | 20 | 2346 |
| | | CUC | | |
| HSV1-UL19-1989 | + | ACACGCACGCCACCGAC | 20 | 2347 |
| | | AAC | | |
| HSV1-UL19-69 | + | UCGUUCAUGUAGGCCAG | 20 | 435 |
| | | CUC | | |
| HSV1-UL19-1991 | + | CUCGUUCAUGUAGGCCA | 20 | 2348 |
| | | GCU | | |
| HSV1-UL19-1992 | + | CAAUCAGCGGCUGGUGG | 20 | 2349 |
| | | ACC | | |
| HSV1-UL19-1993 | + | UCGCGGGCAAUCAGCGG | 20 | 2350 |
| | | CUG | | |
| HSV1-UL19-1994 | + | UCGAUGGGGUGCGGGCC | 20 | 2351 |
| | | GUC | | |
| HSV1-UL19-1995 | + | UGUGGGUUGCUCGAUGG | 20 | 2352 |
| | | GGU | | |
| HSV1-UL19-58 | + | AUUGUGUGUGGGUUGCU | 20 | 425 |
| | | CGA | | |
| HSV1-UL19-1997 | + | AAUUGUGUGUGGGUUGC | 20 | 2353 |
| | | UCG | | |
| HSV1-UL19-1998 | + | UCUUGGUCAUGUAAUUG | 20 | 2354 |
| | | UGU | | |
| HSV1-UL19-1999 | + | GCCAGGCUGAAGGCGGC | 20 | 2355 |
| | | GUU | | |
| HSV1-UL19-2000 | + | CGAUGGCCUCGGUGGCC | 20 | 2356 |
| | | AGG | | |
| HSV1-UL19-2001 | + | UCCAGGGCCUCCCCCGU | 20 | 2357 |
| | | GAG | | |
| HSV1-UL19-2002 | + | UCCCGUCCAGGGCCUCC | 20 | 2358 |
| | | CCC | | |
| HSV1-UL19-2003 | + | GCGCCGAUACCCGUCCC | 20 | 2359 |
| | | GUC | | |
| HSV1-UL19-187 | + | CCCGGAGGGUCGCGGUU | 17 | 460 |
| HSV1-UL19-186 | + | UCCCGGAGGGUCGCGGU | 17 | 459 |
| HSV1-UL19-2006 | + | AUCCCGGAGGGUCGCGG | 17 | 2360 |
| HSV1-UL19-2007 | + | GGCAUACCGGUAUCCCG | 17 | 2361 |
| HSV1-UL19-182 | + | GCGGCAUACCGGUAUCC | 17 | 571 |
| HSV1-UL19-2009 | + | CGCGGCAUACCGGUAUC | 17 | 2362 |
| HSV1-UL19-175 | + | CCUCGAUCGUGCUAAGG | 17 | 566 |
| HSV1-UL19-2011 | + | ACCUCGAUCGUGCUAAG | 17 | 2363 |
| HSV1-UL19-174 | + | CCACCUCGAUCGUGCUA | 17 | 565 |
| HSV1-UL19-2013 | + | GCCACCUCGAUCGUGCU | 17 | 2364 |
| HSV1-UL19-172 | + | UGCAUCGGAGCGCACGC | 17 | 563 |
| HSV1-UL19-171 | + | UUGCAUCGGAGCGCACG | 17 | 562 |
| HSV1-UL19-2016 | + | UUUGCAUCGGAGCGCAC | 17 | 2365 |
| HSV1-UL19-170 | + | GUACAGGCUGUUUGCAU | 17 | 450 |
| HSV1-UL19-2018 | + | CGUACAGGCUGUUUGCA | 17 | 2366 |
| HSV1-UL19-2019 | + | ACGACCCCAGCAGGGCG | 17 | 2367 |
| HSV1-UL19-2020 | + | UUGCAAUACGACCCCAG | 17 | 2368 |
| HSV1-UL19-2021 | + | AGAAAGCGCACGAGCGA | 17 | 2369 |
| HSV1-UL19-2022 | + | CGAGCUCCAGAAAGCGC | 17 | 2370 |
| HSV1-UL19-2023 | + | ACCGACAACCCGAGCUC | 17 | 2371 |
| HSV1-UL19-2024 | + | CGCACGCCACCGACAAC | 17 | 2372 |
| HSV1-UL19-163 | + | UUCAUGUAGGCCAGCUC | 17 | 443 |
| HSV1-UL19-2026 | + | GUUCAUGUAGGCCAGCU | 17 | 2373 |
| HSV1-UL19-2027 | + | UCAGCGGCUGGUGGACC | 17 | 2374 |
| HSV1-UL19-2028 | + | CGGGCAAUCAGCGGCUG | 17 | 2375 |
| HSV1-UL19-2029 | + | AUGGGGUGCGGGCCGUC | 17 | 2376 |
| HSV1-UL19-2030 | + | GGGUUGCUCGAUGGGGU | 17 | 2377 |
| HSV1-UL19-152 | + | GUGUGUGGGUUGCUCGA | 17 | 427 |
| HSV1-UL19-2032 | + | UGUGUGUGGGUUGCUCG | 17 | 2378 |
| HSV1-UL19-2033 | + | UGGUCAUGUAAUUGUGU | 17 | 2379 |
| HSV1-UL19-2034 | + | AGGCUGAAGGCGGCGUU | 17 | 2380 |
| HSV1-UL19-2035 | + | UGGCCUCGGUGGCCAGG | 17 | 2381 |
| HSV1-UL19-2036 | + | AGGGCCUCCCCCGUGAG | 17 | 2382 |
| HSV1-UL19-2037 | + | CGUCCAGGGCCUCCCCC | 17 | 2383 |
| HSV1-UL19-2038 | + | CCGAUACCCGUCCCGUC | 17 | 2384 |

Table 1E provides exemplary targeting domains for knocking out the UL19 gene selected according to the second tier parameters. The targeting domains are selected based on location within the coding sequence, but downstream of the first 500 bp of the UL19 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 1E
2nd Tier

| | | | Target | |
|---|---|---|---|---|
| | DNA | | Site | |
| gRNA Name | Strand | Targeting Domain | Length | Seq ID |
| HSV1-UL19-189 | − | CGCAACGUCCAGGCCGUCCU | 20 | 575 |
| HSV1-UL19-2040 | − | CCAGGCCGUCCUCGGGGCGU | 20 | 2385 |
| HSV1-UL19-2041 | − | ACCAGAUGCUGCACGUGCUG | 20 | 2386 |
| HSV1-UL19-194 | − | CCAGAUGCUGCACGUGCUGC | 20 | 580 |
| HSV1-UL19-2043 | − | UGUUGCCGAUGCAACGAUAC | 20 | 2387 |
| HSV1-UL19-2044 | − | GACAACGGCCGCCUGGCCAC | 20 | 2388 |
| HSV1-UL19-2045 | − | CGCCUGGCCACCAGGGUGGC | 20 | 2389 |
| HSV1-UL19-2046 | − | GGCCCGGGCGACCCUGGUCG | 20 | 2390 |
| HSV1-UL19-2047 | − | ACCCUGGUCGCCGAGCUAAA | 20 | 2391 |
| HSV1-UL19-2048 | − | CGAGCUAAAGCGAAGCUUCU | 20 | 2392 |
| HSV1-UL19-2049 | − | UAAAGCGAAGCUUCUGCGAG | 20 | 2393 |
| HSV1-UL19-2050 | − | UCUGCGAGACGAGCUUUUUC | 20 | 2394 |
| HSV1-UL19-2051 | − | CGAGCUUUUUCCUGGGCAAG | 20 | 2395 |
| HSV1-UL19-2052 | − | CUGGGCAAGGCGGGCCACCG | 20 | 2396 |
| HSV1-UL19-211 | − | UGGGCAAGGCGGGCCACCGC | 20 | 597 |
| HSV1-UL19-212 | − | GGGCAAGGCGGGCCACCGCC | 20 | 598 |
| HSV1-UL19-2055 | − | GGGCCACCGCCGGGAGGCCG | 20 | 2397 |
| HSV1-UL19-2056 | − | AGGCCGUCGAGGCCUGGCUC | 20 | 2398 |
| HSV1-UL19-2057 | − | UCUGACGCAUGCCGACACGC | 20 | 2399 |
| HSV1-UL19-2058 | − | CACGCGCGGGCGGCCGGUCG | 20 | 2400 |
| HSV1-UL19-223 | − | ACGCGCGGGCGGCCGGUCGA | 20 | 609 |
| HSV1-UL19-2060 | − | AGCGCCUCCUGCAGUCCUUC | 20 | 2401 |
| HSV1-UL19-2061 | − | UCCUGCAGUCCUUCCUGAAA | 20 | 2402 |
| HSV1-UL19-226 | − | CCUGCAGUCCUUCCUGAAAG | 20 | 612 |
| HSV1-UL19-2063 | − | UGCAGUCCUUCCUGAAAGUG | 20 | 2403 |
| HSV1-UL19-2064 | − | CUUCCUGAAAGUGGAGGACA | 20 | 2404 |
| HSV1-UL19-2065 | − | CGACGUGCCGGUGACGUACG | 20 | 2405 |
| HSV1-UL19-2066 | − | UGACGUACGGCGAGAUGGUC | 20 | 2406 |
| HSV1-UL19-2067 | − | GUACGGCGAGAUGGUCCUGA | 20 | 2407 |
| HSV1-UL19-231 | − | UACGGCGAGAUGGUCCUGAA | 20 | 617 |
| HSV1-UL19-2069 | − | ACCUGGUCACGGCGCUCGUG | 20 | 2408 |
| HSV1-UL19-2070 | − | CUCGUGAUGGGCAAGGCCGU | 20 | 2409 |
| HSV1-UL19-2071 | − | UGGGCAAGGCCGUGCGAAGU | 20 | 2410 |
| HSV1-UL19-2072 | − | CCGUGCGAAGUCUGGACGAC | 20 | 2411 |
| HSV1-UL19-2073 | − | ACGACGUGGGCCGCCACCUG | 20 | 2412 |
| HSV1-UL19-242 | − | CGACGUGGGCCGCCACCUGC | 20 | 628 |
| HSV1-UL19-2075 | − | GCCGCCACCUGCUGGAGAUG | 20 | 2413 |
| HSV1-UL19-243 | − | CCGCCACCUGCUGGAGAUGC | 20 | 629 |
| HSV1-UL19-2077 | − | GCCACCUGCUGGAGAUGCAG | 20 | 2414 |
| HSV1-UL19-244 | − | CCACCUGCUGGAGAUGCAGG | 20 | 630 |
| HSV1-UL19-2079 | − | UGCAGGAGGAGCAGCUCGAC | 20 | 2415 |
| HSV1-UL19-2080 | − | UCGACCUGAACCGGCAGACG | 20 | 2416 |
| HSV1-UL19-2081 | − | CCUGAACCGGCAGACGCUGG | 20 | 2417 |
| HSV1-UL19-2082 | − | CCGGCAGACGCUGGACGAGC | 20 | 2418 |
| HSV1-UL19-2083 | − | GGCAGACGCUGGACGAGCUC | 20 | 2419 |
| HSV1-UL19-2084 | − | CCCAGACGACGCGCGUGCGC | 20 | 2420 |
| HSV1-UL19-2085 | − | CGCGGAUCUGGUGUCCAUCG | 20 | 2421 |
| HSV1-UL19-2086 | − | CGGAUCUGGUGUCCAUCGGC | 20 | 2422 |
| HSV1-UL19-2087 | − | UCGGCGAGAAGCUGGUCUUU | 20 | 2423 |
| HSV1-UL19-251 | − | CGGCGAGAAGCUGGUCUUUC | 20 | 637 |
| HSV1-UL19-2089 | − | AGCUGGUCUUUCUGGAGGCC | 20 | 2424 |
| HSV1-UL19-253 | − | GCUGGUCUUUCUGGAGGCCC | 20 | 639 |
| HSV1-UL19-2091 | − | UGGUCUUUCUGGAGGCCCUG | 20 | 2425 |
| HSV1-UL19-2092 | − | CCAACGUUCCCUACCCCCUG | 20 | 2426 |
| HSV1-UL19-2093 | − | CCUACCCCCUGGUGGGCGCC | 20 | 2427 |
| HSV1-UL19-2094 | − | ACCUGACGUUCGUCCUGCCC | 20 | 2428 |
| HSV1-UL19-2095 | − | UGGGCCUGUUCAAUCCGGUC | 20 | 2429 |
| HSV1-UL19-261 | − | GGGCCUGUUCAAUCCGGUCA | 20 | 647 |
| HSV1-UL19-2097 | − | GGAACGGUUUGCCGCGCACG | 20 | 2430 |
| HSV1-UL19-263 | − | GAACGGUUUGCCGCGCACGC | 20 | 649 |
| HSV1-UL19-264 | − | AACGGUUUGCCGCGCACGCC | 20 | 650 |
| HSV1-UL19-2100 | − | UAGUCCCCGCCCCCGGCCAC | 20 | 2431 |
| HSV1-UL19-2101 | − | CCGCCCCGCCAGCUGUUUUU | 20 | 2432 |
| HSV1-UL19-268 | − | CGCCCCGCCAGCUGUUUUUU | 20 | 654 |
| HSV1-UL19-269 | − | GCCCCGCCAGCUGUUUUUUU | 20 | 655 |
| HSV1-UL19-270 | − | CCCCGCCAGCUGUUUUUUUG | 20 | 656 |
| HSV1-UL19-271 | − | CCCGCCAGCUGUUUUUUUGG | 20 | 657 |
| HSV1-UL19-2106 | − | GCCAGCUGUUUUUUUGGGGG | 20 | 2433 |
| HSV1-UL19-2107 | − | GCCAGGUGCUGCGCCUGUCU | 20 | 2434 |
| HSV1-UL19-274 | − | CCAGGUGCUGCGCCUGUCUC | 20 | 660 |
| HSV1-UL19-2109 | − | CCUGUCUCUGGAACACGCGA | 20 | 2435 |
| HSV1-UL19-275 | − | CUGUCUCUGGAACACGCGAU | 20 | 661 |
| HSV1-UL19-2111 | − | CCGUGUGCCACCCUUCGCUG | 20 | 2436 |
| HSV1-UL19-2112 | − | GAUGAACGUUGACGCGGCGG | 20 | 2437 |
| HSV1-UL19-279 | − | AUGAACGUUGACGCGGCGGU | 20 | 665 |
| HSV1-UL19-280 | − | UGAACGUUGACGCGGCGGUC | 20 | 666 |
| HSV1-UL19-2115 | − | GGGCCUUAACCGCGACCCCG | 20 | 2438 |
| HSV1-UL19-2116 | − | CGUCGAAGCCGCCAAUCCGU | 20 | 2439 |
| HSV1-UL19-283 | − | GUCGAAGCCGCCAAUCCGUA | 20 | 669 |
| HSV1-UL19-2118 | − | CAGACAUGCAGCAGCUGUUU | 20 | 2440 |
| HSV1-UL19-2119 | − | CAGCAGCUGUUUUUGAACGC | 20 | 2441 |
| HSV1-UL19-290 | − | AGCAGCUGUUUUUGAACGCC | 20 | 676 |
| HSV1-UL19-291 | − | GCAGCUGUUUUUGAACGCCU | 20 | 677 |
| HSV1-UL19-2122 | − | CUGGGGGCAGCGCCUGGCCC | 20 | 2442 |
| HSV1-UL19-2123 | − | GGGCAGCGCCUGGCCCACGG | 20 | 2443 |
| HSV1-UL19-2124 | − | CUGGCCCACGGGCGGGUCCG | 20 | 2444 |
| HSV1-UL19-2125 | − | ACGGGCGGGUCCGAUGGGUC | 20 | 2445 |
| HSV1-UL19-301 | − | CGGGCGGGUCCGAUGGGUCG | 20 | 687 |
| HSV1-UL19-2127 | − | UCGCGGAAGGCCAGAUGACC | 20 | 2446 |
| HSV1-UL19-303 | − | CGCGGAAGGCCAGAUGACCC | 20 | 689 |
| HSV1-UL19-2129 | − | CGACAACGCCAACCUGGCUC | 20 | 2447 |
| HSV1-UL19-2130 | − | ACCCCGCGUUCGACUUCUUU | 20 | 2448 |
| HSV1-UL19-305 | − | CCCCGCGUUCGACUUCUUUG | 20 | 691 |
| HSV1-UL19-306 | − | CCCGCGUUCGACUUCUUUGU | 20 | 692 |
| HSV1-UL19-2133 | − | UCUUUGUGGGGGUGGCCGAC | 20 | 2449 |
| HSV1-UL19-310 | − | CUUUGUGGGGGUGGCCGACG | 20 | 696 |
| HSV1-UL19-2135 | − | GGGUGGCCGACGUGGAGCUG | 20 | 2450 |
| HSV1-UL19-311 | − | GGUGGCCGACGUGGAGCUGC | 20 | 697 |
| HSV1-UL19-312 | − | GUGGCCGACGUGGAGCUGCC | 20 | 698 |
| HSV1-UL19-313 | − | UGGCCGACGUGGAGCUGCCG | 20 | 699 |
| HSV1-UL19-314 | − | GGCCGACGUGGAGCUGCCGG | 20 | 700 |
| HSV1-UL19-315 | − | GCCGACGUGGAGCUGCCGGG | 20 | 701 |
| HSV1-UL19-316 | − | CCGACGUGGAGCUGCCGGGG | 20 | 702 |
| HSV1-UL19-2142 | − | GGGACGUUCCCCCGGCCGGC | 20 | 2451 |
| HSV1-UL19-320 | − | GGACGUUCCCCCGGCCGGCC | 20 | 706 |
| HSV1-UL19-321 | − | GACGUUCCCCCGGCCGGCCC | 20 | 707 |
| HSV1-UL19-322 | − | ACGUUCCCCCGGCCGGCCCG | 20 | 708 |
| HSV1-UL19-323 | − | CGUUCCCCCGGCCGGCCCGG | 20 | 709 |
| HSV1-UL19-2147 | − | UCCAGGCCACCUGGCGCGUG | 20 | 2452 |
| HSV1-UL19-2148 | − | GCGCUAUGUCCGGCGGCGUU | 20 | 2453 |
| HSV1-UL19-331 | − | CGCUAUGUCCGGCGGCGUUC | 20 | 717 |
| HSV1-UL19-2150 | − | CCGGCGGCGUUCCGGGACGC | 20 | 2454 |
| HSV1-UL19-333 | − | CGGCGGCGUUCCGGGACGCC | 20 | 719 |
| HSV1-UL19-2152 | − | CGUUCCGGGACGCCCGGGGC | 20 | 2455 |
| HSV1-UL19-336 | − | GUUCCGGGACGCCCGGGGCC | 20 | 722 |
| HSV1-UL19-2154 | − | GGGACGCCCGGGGCCUGGAG | 20 | 2456 |
| HSV1-UL19-337 | − | GGACGCCCGGGGCCUGGAGC | 20 | 723 |
| HSV1-UL19-338 | − | GACGCCCGGGGCCUGGAGCU | 20 | 724 |
| HSV1-UL19-2157 | − | CCCGGGGCCUGGAGCUGGGG | 20 | 2457 |
| HSV1-UL19-341 | − | CCGGGGCCUGGAGCUGGGGG | 20 | 727 |
| HSV1-UL19-2159 | − | CGCCACCAUCGCCGCCGUUC | 20 | 2458 |
| HSV1-UL19-344 | − | GCCACCAUCGCCGCCGUUCG | 20 | 730 |
| HSV1-UL19-2161 | − | GCAGGCCGCCAUACACGGCA | 20 | 2459 |
| HSV1-UL19-2162 | − | GUCCAGUGCAUCACCAGCUA | 20 | 2460 |
| HSV1-UL19-354 | − | UCCAGUGCAUCACCAGCUAC | 20 | 740 |
| HSV1-UL19-2164 | − | ACACGCGGUGCGCGGCGUUC | 20 | 2461 |
| HSV1-UL19-2165 | − | CUCGUACGUCGUGACCUACC | 20 | 2462 |
| HSV1-UL19-358 | − | UCGUACGUCGUGACCUACCU | 20 | 744 |
| HSV1-UL19-359 | − | CGUACGUCGUGACCUACCUC | 20 | 745 |
| HSV1-UL19-360 | − | GUACGUCGUGACCUACCUCG | 20 | 746 |
| HSV1-UL19-361 | − | UACGUCGUGACCUACCUCGG | 20 | 747 |
| HSV1-UL19-2170 | − | CUACCUCGGGGGAGACCUUC | 20 | 2463 |
| HSV1-UL19-2171 | − | ACCUCGGGGGAGACCUUCCC | 20 | 2464 |
| HSV1-UL19-362 | − | CCUCGGGGGAGACCUUCCCG | 20 | 748 |
| HSV1-UL19-2173 | − | GAGGAGUGCAUGGCCGUGUA | 20 | 2465 |
| HSV1-UL19-364 | − | AGGAGUGCAUGGCCGUGUAC | 20 | 750 |
| HSV1-UL19-2175 | − | CCGGGACCUGGUGGCGCACG | 20 | 2466 |
| HSV1-UL19-2176 | − | AUGACUUUACCCUGACCGGC | 20 | 2467 |
| HSV1-UL19-372 | − | UGACUUUACCCUGACCGGCC | 20 | 758 |
| HSV1-UL19-2178 | − | UUACCCUGACCGGCCCGGAG | 20 | 2468 |
| HSV1-UL19-2179 | − | CCUGACCGGCCCGGAGCUGG | 20 | 2469 |
| HSV1-UL19-2180 | − | GCUGGGCGGGCAGGCGCAAG | 20 | 2470 |
| HSV1-UL19-2181 | − | GCGGGCAGGCGCAAGCCGAG | 20 | 2471 |
| HSV1-UL19-2182 | − | GCCGAGCUGAAUCACCUAAU | 20 | 2472 |
| HSV1-UL19-2183 | − | GCGCUGCUGCCACCCCUCGU | 20 | 2473 |
| HSV1-UL19-379 | − | CGCUGCUGCCACCCCUCGUG | 20 | 765 |
| HSV1-UL19-2185 | − | CCCUGAUGCGGCGCGCGGCC | 20 | 2474 |
| HSV1-UL19-2186 | − | CUGGACCGCCAUCGCGACUG | 20 | 2475 |
| HSV1-UL19-2187 | − | AUCGCGACUGCCGGGUUAGC | 20 | 2476 |
| HSV1-UL19-386 | − | UCGCGACUGCCGGGUUAGCG | 20 | 772 |
| HSV1-UL19-387 | − | CGCGACUGCCGGGUUAGCGC | 20 | 773 |
| HSV1-UL19-388 | − | GCGACUGCCGGGUUAGCGCG | 20 | 774 |
| HSV1-UL19-2191 | − | CGGCAUGUAACGUGGCGACC | 20 | 2477 |
| HSV1-UL19-2192 | − | CUGCUGCACAACACCCAGGC | 20 | 2478 |
| HSV1-UL19-2193 | − | ACAACACCCAGGCCCGAGCC | 20 | 2479 |
| HSV1-UL19-2194 | − | AGGCCCGAGCCGCGGACGCC | 20 | 2480 |
| HSV1-UL19-2195 | − | GCCGCGGAUGACCGGCCGCA | 20 | 2481 |
| HSV1-UL19-400 | − | CCGCGGAUGACCGGCCGCAC | 20 | 786 |
| HSV1-UL19-401 | − | CGCGGAUGACCGGCCGCACC | 20 | 787 |
| HSV1-UL19-402 | − | GCGGAUGACCGGCCGCACCG | 20 | 788 |
| HSV1-UL19-2199 | − | AUGACCGGCCGCACCGGGGG | 20 | 2482 |
| HSV1-UL19-2200 | − | CGGCCGCACCGGGGGGCGGA | 20 | 2483 |
| HSV1-UL19-2201 | − | GUGAUGGUGCCCGCCUUCUC | 20 | 2484 |
| HSV1-UL19-408 | − | UGAUGGUGCCCGCCUUCUCG | 20 | 794 |
| HSV1-UL19-2203 | − | CGCGGGGCCGCUGCUGCACC | 20 | 2485 |
| HSV1-UL19-411 | − | GCGGGGCCGCUGCUGCACCG | 20 | 797 |
| HSV1-UL19-412 | − | CGGGGCCGCUGCUGCACCGC | 20 | 798 |
| HSV1-UL19-2206 | − | ACCGCGUAUACGCCACCCUG | 20 | 2486 |
| HSV1-UL19-2207 | − | CCCUGCAGAACAUGGUGGUC | 20 | 2487 |
| HSV1-UL19-417 | − | CCUGCAGAACAUGGUGGUCC | 20 | 803 |
| HSV1-UL19-2209 | − | GGUCCCGGAGAUCGCCCCCG | 20 | 2488 |
| HSV1-UL19-2210 | − | UCCCGGAGAUCGCCCCCGGC | 20 | 2489 |
| HSV1-UL19-419 | − | CCCGGAGAUCGCCCCCGGCG | 20 | 805 |
| HSV1-UL19-2212 | − | AGUGCCCCAGCGACCCCGUG | 20 | 2490 |
| HSV1-UL19-2213 | − | GGUCAACGCCAUGUUUCACA | 20 | 2491 |
| HSV1-UL19-2214 | − | UUCACAACGGGCGCGUGGUA | 20 | 2492 |
| HSV1-UL19-2215 | − | CAACGGGCGCGUGGUAGUGG | 20 | 2493 |
| HSV1-UL19-2216 | − | GGUGCUGGCCCACAACAUGG | 20 | 2494 |
| HSV1-UL19-2217 | − | UCUGCUCGGCGGCGCCCGAC | 20 | 2495 |
| HSV1-UL19-2218 | − | CACCAACAUGCGCAUAUUCG | 20 | 2496 |
| HSV1-UL19-439 | − | ACCAACAUGCGCAUAUUCGA | 20 | 825 |
| HSV1-UL19-2220 | − | AUUCGACGGGGCGUUGCACG | 20 | 2497 |
| HSV1-UL19-442 | − | UUCGACGGGGCGUUGCACGC | 20 | 828 |
| HSV1-UL19-2222 | − | UGCUGAUGGCCCCCCAGCAU | 20 | 2498 |
| HSV1-UL19-2223 | − | CCCCGUCCACGCGCUGUUCG | 20 | 2499 |
| HSV1-UL19-446 | − | CCCGUCCACGCGCUGUUCGC | 20 | 832 |
| HSV1-UL19-2225 | − | UCGCCGGGGCCGACCACGUG | 20 | 2500 |
| HSV1-UL19-2226 | − | UCCCCCUGGUCCCCCCGGCU | 20 | 2501 |
| HSV1-UL19-455 | − | CCCCCUGGUCCCCCCGGCUC | 20 | 841 |
| HSV1-UL19-456 | − | CCCCUGGUCCCCCCGGCUCU | 20 | 842 |
| HSV1-UL19-2229 | − | GCCCGUCGUGCAGCACGUCC | 20 | 2502 |
| HSV1-UL19-2230 | − | CCGUCGUGCAGCACGUCCGC | 20 | 2503 |
| HSV1-UL19-2231 | − | GCACGUCCGCGAGAGCGCGG | 20 | 2504 |
| HSV1-UL19-460 | − | CACGUCCGCGAGAGCGCGGC | 20 | 846 |
| HSV1-UL19-461 | − | ACGUCCGCGAGAGCGCGGCC | 20 | 847 |
| HSV1-UL19-462 | − | CGUCCGCGAGAGCGCGGCCG | 20 | 848 |
| HSV1-UL19-2235 | − | UCCGCGAGAGCGCGGCCGGG | 20 | 2505 |
| HSV1-UL19-2236 | − | CGCUGACCUACGCGCUCAUG | 20 | 2506 |
| HSV1-UL19-464 | − | GCUGACCUACGCGCUCAUGG | 20 | 850 |
| HSV1-UL19-2238 | − | CCUUGCAUCAUCAGCUCAAG | 20 | 2507 |
| HSV1-UL19-2239 | − | GCUCAAGACGGGCCUCCAUC | 20 | 2508 |
| HSV1-UL19-2240 | − | GACGGGCCUCCAUCCCGGGU | 20 | 2509 |
| HSV1-UL19-2241 | − | UUGGGUUCACCGUCGUCCGA | 20 | 2510 |
| HSV1-UL19-2242 | − | CCGACAGGACCGCUUUGUGA | 20 | 2511 |
| HSV1-UL19-2243 | − | GACAGGACCGCUUUGUGACU | 20 | 2512 |
| HSV1-UL19-2244 | − | UGACUGAGAACGUGCUGUUC | 20 | 2513 |
| HSV1-UL19-475 | − | GACUGAGAACGUGCUGUUCU | 20 | 861 |
| HSV1-UL19-2246 | − | UGCUGUUCUCGGAGCGCGCG | 20 | 2514 |
| HSV1-UL19-476 | − | GCUGUUCUCGGAGCGCGCGU | 20 | 862 |
| HSV1-UL19-2248 | − | GCGCGUCGGAGGCGUACUUC | 20 | 2515 |
| HSV1-UL19-2249 | − | CCAGCUCCAGGUGGCCCGGC | 20 | 2516 |
| HSV1-UL19-2250 | − | GGUGGCCCGGCACGAAACUG | 20 | 2517 |
| HSV1-UL19-484 | − | GUGGCCCGGCACGAAACUGG | 20 | 870 |
| HSV1-UL19-485 | − | UGGCCCGGCACGAAACUGGC | 20 | 871 |
| HSV1-UL19-486 | − | GGCCCGGCACGAAACUGGCG | 20 | 872 |
| HSV1-UL19-487 | − | GCCCGGCACGAAACUGGCGG | 20 | 873 |
| HSV1-UL19-2255 | − | UCACGCUCACCCAGCCGCGC | 20 | 2518 |
| HSV1-UL19-2256 | − | UCACCCAGCCGCGCGCGAAC | 20 | 2519 |
| HSV1-UL19-2257 | − | AGCCGCGCGCGAACGUGGAC | 20 | 2520 |
| HSV1-UL19-2258 | − | GCGCGAACGUGGACCUGGGC | 20 | 2521 |
| HSV1-UL19-2259 | − | UCCGCAACCCCGUCACCGAC | 20 | 2522 |
| HSV1-UL19-2260 | − | ACCUUCCCCAAAACUUUUAC | 20 | 2523 |
| HSV1-UL19-2261 | − | CCAAAACUUUUACCUGGGCC | 20 | 2524 |
| HSV1-UL19-501 | − | CAAAACUUUUACCUGGGCCG | 20 | 887 |
| HSV1-UL19-2263 | − | GCCGCGGGGCUCCCCCUCUC | 20 | 2525 |
| HSV1-UL19-2264 | − | AACGCGGCAGCCGUGUACCU | 20 | 2526 |
| HSV1-UL19-506 | − | ACGCGGCAGCCGUGUACCUG | 20 | 892 |
| HSV1-UL19-2266 | − | ACCUGCGGAACGCGGUCGUG | 20 | 2527 |
| HSV1-UL19-509 | − | CCUGCGGAACGCGGUCGUGG | 20 | 895 |
| HSV1-UL19-510 | − | CUGCGGAACGCGGUCGUGGC | 20 | 896 |
| HSV1-UL19-2269 | − | CGGUCGUGGCGGGAAACCGC | 20 | 2528 |
| HSV1-UL19-511 | − | GGUCGUGGCGGGAAACCGCC | 20 | 897 |
| HSV1-UL19-2271 | − | GGCCCAGCCCGUCCCCGUGU | 20 | 2529 |
| HSV1-UL19-2272 | − | CGCCCAGGUGCCGCGGCGCG | 20 | 2530 |
| HSV1-UL19-519 | − | GCCCAGGUGCCGCGGCGCGC | 20 | 905 |
| HSV1-UL19-2274 | − | AGGUGCCGCGGCGCGCAGGG | 20 | 2531 |
| HSV1-UL19-2275 | − | GCGCAGGGAUGGACCACGGC | 20 | 2532 |
| HSV1-UL19-2276 | − | CCACGGCCAGGACGCCGUGU | 20 | 2533 |
| HSV1-UL19-2277 | − | UUCCGCCGGCCCUGCAACCC | 20 | 2534 |
| HSV1-UL19-525 | − | UCCGCCGGCCCUGCAACCCC | 20 | 911 |
| HSV1-UL19-526 | − | CCGCCGGCCCUGCAACCCCC | 20 | 912 |
| HSV1-UL19-2280 | − | CAACCCCCGGGGACGCGCCG | 20 | 2535 |
| HSV1-UL19-528 | − | AACCCCCGGGGACGCGCCGC | 20 | 914 |
| HSV1-UL19-529 | − | ACCCCCGGGGACGCGCCGCC | 20 | 915 |
| HSV1-UL19-2283 | − | GCGCCGCCGGGGGCGUUUAC | 20 | 2536 |
| HSV1-UL19-532 | − | CGCCGCCGGGGGCGUUUACG | 20 | 918 |
| HSV1-UL19-533 | − | GCCGCCGGGGGCGUUUACGC | 20 | 919 |
| HSV1-UL19-534 | − | CCGCCGGGGGCGUUUACGCG | 20 | 920 |
| HSV1-UL19-2287 | − | GGGGCGUUUACGCGGGGGAC | 20 | 2537 |
| HSV1-UL19-536 | − | GGGCGUUUACGCGGGGGACA | 20 | 922 |
| HSV1-UL19-2289 | − | GCGUUUACGCGGGGGACAAG | 20 | 2538 |
| HSV1-UL19-537 | − | CGUUUACGCGGGGGACAAGG | 20 | 923 |
| HSV1-UL19-538 | − | GUUUACGCGGGGGACAAGGA | 20 | 924 |
| HSV1-UL19-539 | − | UUUACGCGGGGGACAAGGAG | 20 | 925 |
| HSV1-UL19-2293 | − | CCCUCAUGUACGACCACGGC | 20 | 2539 |
| HSV1-UL19-2294 | − | CACGGCCAGAGCGACCCGUC | 20 | 2540 |
| HSV1-UL19-2295 | − | UUCGCGGCCACGGCCAACCC | 20 | 2541 |
| HSV1-UL19-2296 | − | GGCGUCGCAGCGAUUUUCGU | 20 | 2542 |
| HSV1-UL19-548 | − | GCGUCGCAGCGAUUUUCGUA | 20 | 934 |
| HSV1-UL19-549 | − | CGUCGCAGCGAUUUUCGUAC | 20 | 935 |
| HSV1-UL19-2299 | − | GUACGGGGACCUGCUCUAUA | 20 | 2543 |
| HSV1-UL19-551 | − | UACGGGGACCUGCUCUAUAA | 20 | 937 |
| HSV1-UL19-2301 | − | UAACGGGGCCUACCACCUCA | 20 | 2544 |
| HSV1-UL19-554 | − | AACGGGGCCUACCACCUCAA | 20 | 940 |
| HSV1-UL19-2303 | − | UCGCCGCCAAACAUCGCUGC | 20 | 2545 |
| HSV1-UL19-559 | − | CGCCGCCAAACAUCGCUGCC | 20 | 945 |
| HSV1-UL19-2305 | − | GCUGCCUGGAGCGCCUGAUC | 20 | 2546 |
| HSV1-UL19-560 | − | CUGCCUGGAGCGCCUGAUCG | 20 | 946 |
| HSV1-UL19-2307 | − | UGGAGCGCCUGAUCGUGGAG | 20 | 2547 |
| HSV1-UL19-2308 | − | ACGUACAGUUUAAGCGCCCC | 20 | 2548 |
| HSV1-UL19-566 | − | CGUACAGUUUAAGCGCCCCC | 20 | 952 |
| HSV1-UL19-2310 | − | UAAGCGCCCCCCGGGGUGCC | 20 | 2549 |
| HSV1-UL19-2311 | − | CCCCGGGGUGCCGCGAACUC | 20 | 2550 |
| HSV1-UL19-569 | − | CCCGGGGUGCCGCGAACUCG | 20 | 955 |
| HSV1-UL19-2313 | − | CGGGGUGCCGCGAACUCGUG | 20 | 2551 |
| HSV1-UL19-2314 | − | AGGACCCGUGUGGCCUGUUU | 20 | 2552 |
| HSV1-UL19-572 | − | GGACCCGUGUGGCCUGUUUC | 20 | 958 |
| HSV1-UL19-2316 | − | CCUCCUCCGCAGUGCCCGCA | 20 | 2553 |
| HSV1-UL19-574 | − | CUCCUCCGCAGUGCCCGCAA | 20 | 960 |
| HSV1-UL19-575 | − | UCCUCCGCAGUGCCCGCAAC | 20 | 961 |
| HSV1-UL19-576 | − | CCUCCGCAGUGCCCGCAACG | 20 | 962 |
| HSV1-UL19-2320 | − | CGCAACGGGGAAGCCCACGC | 20 | 2554 |
| HSV1-UL19-577 | − | GCAACGGGGAAGCCCACGCG | 20 | 963 |
| HSV1-UL19-578 | − | CAACGGGGAAGCCCACGCGC | 20 | 964 |
| HSV1-UL19-2323 | − | UCUAUGACGCAUCCCCGCUC | 20 | 2555 |
| HSV1-UL19-579 | − | CUAUGACGCAUCCCCGCUCA | 20 | 965 |
| HSV1-UL19-582 | − | AACGUCCAGGCCGUCCU | 17 | 968 |
| HSV1-UL19-2326 | − | GGCCGUCCUCGGGGCGU | 17 | 2556 |
| HSV1-UL19-2327 | − | AGAUGCUGCACGUGCUG | 17 | 2557 |
| HSV1-UL19-587 | − | GAUGCUGCACGUGCUGC | 17 | 973 |
| HSV1-UL19-2329 | − | UGCCGAUGCAACGAUAC | 17 | 2558 |
| HSV1-UL19-2330 | − | AACGGCCGCCUGGCCAC | 17 | 2559 |
| HSV1-UL19-2331 | − | CUGGCCACCAGGGUGGC | 17 | 2560 |
| HSV1-UL19-2332 | − | CCGGGCGACCCUGGUCG | 17 | 2561 |
| HSV1-UL19-2333 | − | CUGGUCGCCGAGCUAAA | 17 | 2562 |
| HSV1-UL19-2334 | − | GCUAAAGCGAAGCUUCU | 17 | 2563 |
| HSV1-UL19-2335 | − | AGCGAAGCUUCUGCGAG | 17 | 2564 |
| HSV1-UL19-2336 | − | GCGAGACGAGCUUUUUC | 17 | 2565 |
| HSV1-UL19-2337 | − | GCUUUUUCCUGGGCAAG | 17 | 2566 |
| HSV1-UL19-2338 | − | GGCAAGGCGGGCCACCG | 17 | 2567 |
| HSV1-UL19-604 | − | GCAAGGCGGGCCACCGC | 17 | 990 |
| HSV1-UL19-605 | − | CAAGGCGGGCCACCGCC | 17 | 991 |
| HSV1-UL19-2341 | − | CCACCGCCGGGAGGCCG | 17 | 2568 |
| HSV1-UL19-2342 | − | CCGUCGAGGCCUGGCUC | 17 | 2569 |
| HSV1-UL19-2343 | − | GACGCAUGCCGACACGC | 17 | 2570 |
| HSV1-UL19-2344 | − | GCGCGGGCGGCCGGUCG | 17 | 2571 |
| HSV1-UL19-616 | − | CGCGGGCGGCCGGUCGA | 17 | 1002 |
| HSV1-UL19-2346 | − | GCCUCCUGCAGUCCUUC | 17 | 2572 |
| HSV1-UL19-2347 | − | UGCAGUCCUUCCUGAAA | 17 | 2573 |
| HSV1-UL19-619 | − | GCAGUCCUUCCUGAAAG | 17 | 1005 |
| HSV1-UL19-2349 | − | AGUCCUUCCUGAAAGUG | 17 | 2574 |
| HSV1-UL19-2350 | − | CCUGAAAGUGGAGGACA | 17 | 2575 |
| HSV1-UL19-2351 | − | CGUGCCGGUGACGUACG | 17 | 2576 |
| HSV1-UL19-2352 | − | CGUACGGCGAGAUGGUC | 17 | 2577 |
| HSV1-UL19-2353 | − | CGGCGAGAUGGUCCUGA | 17 | 2578 |
| HSV1-UL19-624 | − | GGCGAGAUGGUCCUGAA | 17 | 1010 |
| HSV1-UL19-2355 | − | UGGUCACGGCGCUCGUG | 17 | 2579 |
| HSV1-UL19-2356 | − | GUGAUGGGCAAGGCCGU | 17 | 2580 |
| HSV1-UL19-2357 | − | GCAAGGCCGUGCGAAGU | 17 | 2581 |
| HSV1-UL19-2358 | − | UGCGAAGUCUGGACGAC | 17 | 2582 |
| HSV1-UL19-2359 | − | ACGUGGGCCGCCACCUG | 17 | 2583 |
| HSV1-UL19-635 | − | CGUGGGCCGCCACCUGC | 17 | 1021 |
| HSV1-UL19-2361 | − | GCCACCUGCUGGAGAUG | 17 | 2584 |
| HSV1-UL19-636 | − | CCACCUGCUGGAGAUGC | 17 | 1022 |
| HSV1-UL19-2363 | − | ACCUGCUGGAGAUGCAG | 17 | 2585 |
| HSV1-UL19-637 | − | CCUGCUGGAGAUGCAGG | 17 | 1023 |
| HSV1-UL19-2365 | − | AGGAGGAGCAGCUCGAC | 17 | 2586 |
| HSV1-UL19-2366 | − | ACCUGAACCGGCAGACG | 17 | 2587 |
| HSV1-UL19-2367 | − | GAACCGGCAGACGCUGG | 17 | 2588 |
| HSV1-UL19-2368 | − | GCAGACGCUGGACGAGC | 17 | 2589 |
| HSV1-UL19-2369 | − | AGACGCUGGACGAGCUC | 17 | 2590 |
| HSV1-UL19-2370 | − | AGACGACGCGCGUGCGC | 17 | 2591 |
| HSV1-UL19-2371 | − | GGAUCUGGUGUCCAUCG | 17 | 2592 |
| HSV1-UL19-2372 | − | AUCUGGUGUCCAUCGGC | 17 | 2593 |
| HSV1-UL19-2373 | − | GCGAGAAGCUGGUCUUU | 17 | 2594 |
| HSV1-UL19-644 | − | CGAGAAGCUGGUCUUUC | 17 | 1030 |
| HSV1-UL19-2375 | − | UGGUCUUUCUGGAGGCC | 17 | 2595 |
| HSV1-UL19-646 | − | GGUCUUUCUGGAGGCCC | 17 | 1032 |
| HSV1-UL19-2377 | − | UCUUUCUGGAGGCCCUG | 17 | 2596 |
| HSV1-UL19-2378 | − | ACGUUCCCUACCCCCUG | 17 | 2597 |
| HSV1-UL19-2379 | − | ACCCCCUGGUGGGCGCC | 17 | 2598 |
| HSV1-UL19-2380 | − | UGACGUUCGUCCUGCCC | 17 | 2599 |
| HSV1-UL19-2381 | − | GCCUGUUCAAUCCGGUC | 17 | 2600 |
| HSV1-UL19-654 | − | CCUGUUCAAUCCGGUCA | 17 | 1040 |
| HSV1-UL19-2383 | − | ACGGUUUGCCGCGCACG | 17 | 2601 |
| HSV1-UL19-656 | − | CGGUUUGCCGCGCACGC | 17 | 1042 |
| HSV1-UL19-657 | − | GGUUUGCCGCGCACGCC | 17 | 1043 |
| HSV1-UL19-2386 | − | UCCCCGCCCCCGGCCAC | 17 | 2602 |
| HSV1-UL19-2387 | − | CCCCGCCAGCUGUUUUU | 17 | 2603 |
| HSV1-UL19-661 | − | CCCGCCAGCUGUUUUUU | 17 | 1047 |
| HSV1-UL19-662 | − | CCGCCAGCUGUUUUUUU | 17 | 1048 |
| HSV1-UL19-663 | − | CGCCAGCUGUUUUUUUG | 17 | 1049 |
| HSV1-UL19-664 | − | GCCAGCUGUUUUUUUGG | 17 | 1050 |
| HSV1-UL19-2392 | − | AGCUGUUUUUUUGGGGG | 17 | 2604 |
| HSV1-UL19-2393 | − | AGGUGCUGCGCCUGUCU | 17 | 2605 |
| HSV1-UL19-667 | − | GGUGCUGCGCCUGUCUC | 17 | 1053 |
| HSV1-UL19-2395 | − | GUCUCUGGAACACGCGA | 17 | 2606 |
| HSV1-UL19-668 | − | UCUCUGGAACACGCGAU | 17 | 1054 |
| HSV1-UL19-2397 | − | UGUGCCACCCUUCGCUG | 17 | 2607 |
| HSV1-UL19-2398 | − | GAACGUUGACGCGGCGG | 17 | 2608 |
| HSV1-UL19-672 | − | AACGUUGACGCGGCGGU | 17 | 1058 |
| HSV1-UL19-673 | − | ACGUUGACGCGGCGGUC | 17 | 1059 |
| HSV1-UL19-2401 | − | CCUUAACCGCGACCCCG | 17 | 2609 |
| HSV1-UL19-2402 | − | CGAAGCCGCCAAUCCGU | 17 | 2610 |
| HSV1-UL19-676 | − | GAAGCCGCCAAUCCGUA | 17 | 1062 |
| HSV1-UL19-2404 | − | ACAUGCAGCAGCUGUUU | 17 | 2611 |
| HSV1-UL19-2405 | − | CAGCUGUUUUUGAACGC | 17 | 2612 |
| HSV1-UL19-683 | − | AGCUGUUUUUGAACGCC | 17 | 1069 |
| HSV1-UL19-684 | − | GCUGUUUUUGAACGCCU | 17 | 1070 |
| HSV1-UL19-2408 | − | GGGGCAGCGCCUGGCCC | 17 | 2613 |
| HSV1-UL19-2409 | − | CAGCGCCUGGCCCACGG | 17 | 2614 |
| HSV1-UL19-2410 | − | GCCCACGGGCGGGUCCG | 17 | 2615 |
| HSV1-UL19-2411 | − | GGCGGGUCCGAUGGGUC | 17 | 2616 |
| HSV1-UL19-694 | − | GCGGGUCCGAUGGGUCG | 17 | 1080 |
| HSV1-UL19-2413 | − | CGGAAGGCCAGAUGACC | 17 | 2617 |
| HSV1-UL19-696 | − | GGAAGGCCAGAUGACCC | 17 | 1082 |
| HSV1-UL19-2415 | − | CAACGCCAACCUGGCUC | 17 | 2618 |
| HSV1-UL19-2416 | − | CCGCGUUCGACUUCUUU | 17 | 2619 |
| HSV1-UL19-698 | − | CGCGUUCGACUUCUUUG | 17 | 1084 |
| HSV1-UL19-699 | − | GCGUUCGACUUCUUUGU | 17 | 1085 |
| HSV1-UL19-2419 | − | UUGUGGGGGUGGCCGAC | 17 | 2620 |
| HSV1-UL19-703 | − | UGUGGGGGUGGCCGACG | 17 | 1089 |
| HSV1-UL19-2421 | − | UGGCCGACGUGGAGCUG | 17 | 2621 |
| HSV1-UL19-704 | − | GGCCGACGUGGAGCUGC | 17 | 1090 |
| HSV1-UL19-705 | − | GCCGACGUGGAGCUGCC | 17 | 1091 |
| HSV1-UL19-706 | − | CCGACGUGGAGCUGCCG | 17 | 1092 |
| HSV1-UL19-707 | − | CGACGUGGAGCUGCCGG | 17 | 1093 |
| HSV1-UL19-708 | − | GACGUGGAGCUGCCGGG | 17 | 1094 |
| HSV1-UL19-709 | − | ACGUGGAGCUGCCGGGG | 17 | 1095 |
| HSV1-UL19-2428 | − | ACGUUCCCCCGGCCGGC | 17 | 2622 |
| HSV1-UL19-713 | − | CGUUCCCCCGGCCGGCC | 17 | 1099 |
| HSV1-UL19-714 | − | GUUCCCCCGGCCGGCCC | 17 | 1100 |
| HSV1-UL19-715 | − | UUCCCCCGGCCGGCCCG | 17 | 1101 |
| HSV1-UL19-716 | − | UCCCCCGGCCGGCCCGG | 17 | 1102 |
| HSV1-UL19-2433 | − | AGGCCACCUGGCGCGUG | 17 | 2623 |
| HSV1-UL19-2434 | − | CUAUGUCCGGCGGCGUU | 17 | 2624 |
| HSV1-UL19-724 | − | UAUGUCCGGCGGCGUUC | 17 | 1110 |
| HSV1-UL19-2436 | − | GCGGCGUUCCGGGACGC | 17 | 2625 |
| HSV1-UL19-726 | − | CGGCGUUCCGGGACGCC | 17 | 1112 |
| HSV1-UL19-2438 | − | UCCGGGACGCCCGGGGC | 17 | 2626 |
| HSV1-UL19-729 | − | CCGGGACGCCCGGGGCC | 17 | 1115 |
| HSV1-UL19-2440 | − | ACGCCCGGGGCCUGGAG | 17 | 2627 |
| HSV1-UL19-730 | − | CGCCCGGGGCCUGGAGC | 17 | 1116 |
| HSV1-UL19-731 | − | GCCCGGGGCCUGGAGCU | 17 | 1117 |
| HSV1-UL19-2443 | − | GGGGCCUGGAGCUGGGG | 17 | 2628 |
| HSV1-UL19-734 | − | GGGCCUGGAGCUGGGGG | 17 | 1120 |
| HSV1-UL19-2445 | − | CACCAUCGCCGCCGUUC | 17 | 2629 |
| HSV1-UL19-737 | − | ACCAUCGCCGCCGUUCG | 17 | 1123 |
| HSV1-UL19-2447 | − | GGCCGCCAUACACGGCA | 17 | 2630 |
| HSV1-UL19-2448 | − | CAGUGCAUCACCAGCUA | 17 | 2631 |
| HSV1-UL19-747 | − | AGUGCAUCACCAGCUAC | 17 | 1133 |
| HSV1-UL19-2450 | − | CGCGGUGCGCGGCGUUC | 17 | 2632 |
| HSV1-UL19-2451 | − | GUACGUCGUGACCUACC | 17 | 2633 |
| HSV1-UL19-751 | − | UACGUCGUGACCUACCU | 17 | 1137 |
| HSV1-UL19-752 | − | ACGUCGUGACCUACCUC | 17 | 1138 |
| HSV1-UL19-753 | − | CGUCGUGACCUACCUCG | 17 | 1139 |
| HSV1-UL19-754 | − | GUCGUGACCUACCUCGG | 17 | 1140 |
| HSV1-UL19-2456 | − | CCUCGGGGGAGACCUUC | 17 | 2634 |
| HSV1-UL19-2457 | − | UCGGGGGAGACCUUCCC | 17 | 2635 |
| HSV1-UL19-755 | − | CGGGGGAGACCUUCCCG | 17 | 1141 |
| HSV1-UL19-2459 | − | GAGUGCAUGGCCGUGUA | 17 | 2636 |
| HSV1-UL19-757 | − | AGUGCAUGGCCGUGUAC | 17 | 1143 |
| HSV1-UL19-2461 | − | GGACCUGGUGGCGCACG | 17 | 2637 |
| HSV1-UL19-2462 | − | ACUUUACCCUGACCGGC | 17 | 2638 |
| HSV1-UL19-765 | − | CUUUACCCUGACCGGCC | 17 | 1151 |
| HSV1-UL19-2464 | − | CCCUGACCGGCCCGGAG | 17 | 2639 |
| HSV1-UL19-2465 | − | GACCGGCCCGGAGCUGG | 17 | 2640 |
| HSV1-UL19-2466 | − | GGGCGGGCAGGCGCAAG | 17 | 2641 |
| HSV1-UL19-2467 | − | GGCAGGCGCAAGCCGAG | 17 | 2642 |
| HSV1-UL19-2468 | − | GAGCUGAAUCACCUAAU | 17 | 2643 |
| HSV1-UL19-2469 | − | CUGCUGCCACCCCUCGU | 17 | 2644 |
| HSV1-UL19-772 | − | UGCUGCCACCCCUCGUG | 17 | 1158 |
| HSV1-UL19-2471 | − | UGAUGCGGCGCGCGGCC | 17 | 2645 |
| HSV1-UL19-2472 | − | GACCGCCAUCGCGACUG | 17 | 2646 |
| HSV1-UL19-2473 | − | GCGACUGCCGGGUUAGC | 17 | 2647 |
| HSV1-UL19-779 | − | CGACUGCCGGGUUAGCG | 17 | 1165 |
| HSV1-UL19-780 | − | GACUGCCGGGUUAGCGC | 17 | 1166 |
| HSV1-UL19-781 | − | ACUGCCGGGUUAGCGCG | 17 | 1167 |
| HSV1-UL19-2477 | − | CAUGUAACGUGGCGACC | 17 | 2648 |
| HSV1-UL19-2478 | − | CUGCACAACACCCAGGC | 17 | 2649 |
| HSV1-UL19-2479 | − | ACACCCAGGCCCGAGCC | 17 | 2650 |
| HSV1-UL19-2480 | − | CCCGAGCCGCGGACGCC | 17 | 2651 |
| HSV1-UL19-2481 | − | GCGGAUGACCGGCCGCA | 17 | 2652 |
| HSV1-UL19-793 | − | CGGAUGACCGGCCGCAC | 17 | 1179 |
| HSV1-UL19-794 | − | GGAUGACCGGCCGCACC | 17 | 1180 |
| HSV1-UL19-795 | − | GAUGACCGGCCGCACCG | 17 | 1181 |
| HSV1-UL19-2485 | − | ACCGGCCGCACCGGGGG | 17 | 2653 |
| HSV1-UL19-2486 | − | CCGCACCGGGGGGCGGA | 17 | 2654 |
| HSV1-UL19-2487 | − | AUGGUGCCCGCCUUCUC | 17 | 2655 |
| HSV1-UL19-801 | − | UGGUGCCCGCCUUCUCG | 17 | 1187 |
| HSV1-UL19-2489 | − | GGGGCCGCUGCUGCACC | 17 | 2656 |
| HSV1-UL19-804 | − | GGGCCGCUGCUGCACCG | 17 | 1190 |
| HSV1-UL19-805 | − | GGCCGCUGCUGCACCGC | 17 | 1191 |
| HSV1-UL19-2492 | − | GCGUAUACGCCACCCUG | 17 | 2657 |
| HSV1-UL19-2493 | − | UGCAGAACAUGGUGGUC | 17 | 2658 |
| HSV1-UL19-810 | − | GCAGAACAUGGUGGUCC | 17 | 1196 |
| HSV1-UL19-2495 | − | CCCGGAGAUCGCCCCCG | 17 | 2659 |
| HSV1-UL19-2496 | − | CGGAGAUCGCCCCCGGC | 17 | 2660 |
| HSV1-UL19-812 | − | GGAGAUCGCCCCCGGCG | 17 | 1198 |
| HSV1-UL19-2498 | − | GCCCCAGCGACCCCGUG | 17 | 2661 |
| HSV1-UL19-2499 | − | CAACGCCAUGUUUCACA | 17 | 2662 |
| HSV1-UL19-2500 | − | ACAACGGGCGCGUGGUA | 17 | 2663 |
| HSV1-UL19-2501 | − | CGGGCGCGUGGUAGUGG | 17 | 2664 |
| HSV1-UL19-2502 | − | GCUGGCCCACAACAUGG | 17 | 2665 |
| HSV1-UL19-2503 | − | GCUCGGCGGCGCCCGAC | 17 | 2666 |
| HSV1-UL19-2504 | − | CAACAUGCGCAUAUUCG | 17 | 2667 |
| HSV1-UL19-832 | − | AACAUGCGCAUAUUCGA | 17 | 1218 |
| HSV1-UL19-2506 | − | CGACGGGGCGUUGCACG | 17 | 2668 |
| HSV1-UL19-835 | − | GACGGGGCGUUGCACGC | 17 | 1221 |
| HSV1-UL19-2508 | − | UGAUGGCCCCCCAGCAU | 17 | 2669 |
| HSV1-UL19-2509 | − | CGUCCACGCGCUGUUCG | 17 | 2670 |
| HSV1-UL19-839 | − | GUCCACGCGCUGUUCGC | 17 | 1225 |
| HSV1-UL19-2511 | − | CCGGGGCCGACCACGUG | 17 | 2671 |
| HSV1-UL19-2512 | − | CCCUGGUCCCCCCGGCU | 17 | 2672 |
| HSV1-UL19-848 | − | CCUGGUCCCCCCGGCUC | 17 | 1234 |
| HSV1-UL19-849 | − | CUGGUCCCCCCGGCUCU | 17 | 1235 |
| HSV1-UL19-2515 | − | CGUCGUGCAGCACGUCC | 17 | 2673 |
| HSV1-UL19-2516 | − | UCGUGCAGCACGUCCGC | 17 | 2674 |
| HSV1-UL19-2517 | − | CGUCCGCGAGAGCGCGG | 17 | 2675 |
| HSV1-UL19-853 | − | GUCCGCGAGAGCGCGGC | 17 | 1239 |
| HSV1-UL19-854 | − | UCCGCGAGAGCGCGGCC | 17 | 1240 |
| HSV1-UL19-855 | − | CCGCGAGAGCGCGGCCG | 17 | 1241 |
| HSV1-UL19-2521 | − | GCGAGAGCGCGGCCGGG | 17 | 2676 |
| HSV1-UL19-2522 | − | UGACCUACGCGCUCAUG | 17 | 2677 |
| HSV1-UL19-857 | − | GACCUACGCGCUCAUGG | 17 | 1243 |
| HSV1-UL19-2524 | − | UGCAUCAUCAGCUCAAG | 17 | 2678 |
| HSV1-UL19-2525 | − | CAAGACGGGCCUCCAUC | 17 | 2679 |
| HSV1-UL19-2526 | − | GGGCCUCCAUCCCGGGU | 17 | 2680 |
| HSV1-UL19-2527 | − | GGUUCACCGUCGUCCGA | 17 | 2681 |
| HSV1-UL19-2528 | − | ACAGGACCGCUUUGUGA | 17 | 2682 |
| HSV1-UL19-2529 | − | AGGACCGCUUUGUGACU | 17 | 2683 |
| HSV1-UL19-2530 | − | CUGAGAACGUGCUGUUC | 17 | 2684 |
| HSV1-UL19-868 | − | UGAGAACGUGCUGUUCU | 17 | 1254 |
| HSV1-UL19-2532 | − | UGUUCUCGGAGCGCGCG | 17 | 2685 |
| HSV1-UL19-869 | − | GUUCUCGGAGCGCGCGU | 17 | 1255 |
| HSV1-UL19-2534 | − | CGUCGGAGGCGUACUUC | 17 | 2686 |
| HSV1-UL19-2535 | − | GCUCCAGGUGGCCCGGC | 17 | 2687 |
| HSV1-UL19-2536 | − | GGCCCGGCACGAAACUG | 17 | 2688 |
| HSV1-UL19-877 | − | GCCCGGCACGAAACUGG | 17 | 1263 |
| HSV1-UL19-878 | − | CCCGGCACGAAACUGGC | 17 | 1264 |
| HSV1-UL19-879 | − | CCGGCACGAAACUGGCG | 17 | 1265 |
| HSV1-UL19-880 | − | CGGCACGAAACUGGCGG | 17 | 1266 |
| HSV1-UL19-2541 | − | CGCUCACCCAGCCGCGC | 17 | 2689 |
| HSV1-UL19-2542 | − | CCCAGCCGCGCGCGAAC | 17 | 2690 |
| HSV1-UL19-2543 | − | CGCGCGCGAACGUGGAC | 17 | 2691 |
| HSV1-UL19-2544 | − | CGAACGUGGACCUGGGC | 17 | 2692 |
| HSV1-UL19-2545 | − | GCAACCCCGUCACCGAC | 17 | 2693 |
| HSV1-UL19-2546 | − | UUCCCCAAAACUUUUAC | 17 | 2694 |
| HSV1-UL19-2547 | − | AAACUUUUACCUGGGCC | 17 | 2695 |
| HSV1-UL19-894 | − | AACUUUUACCUGGGCCG | 17 | 1280 |
| HSV1-UL19-2549 | − | GCGGGGCUCCCCCUCUC | 17 | 2696 |
| HSV1-UL19-2550 | − | GCGGCAGCCGUGUACCU | 17 | 2697 |
| HSV1-UL19-899 | − | CGGCAGCCGUGUACCUG | 17 | 1285 |
| HSV1-UL19-2552 | − | UGCGGAACGCGGUCGUG | 17 | 2698 |
| HSV1-UL19-902 | − | GCGGAACGCGGUCGUGG | 17 | 1288 |
| HSV1-UL19-903 | − | CGGAACGCGGUCGUGGC | 17 | 1289 |
| HSV1-UL19-2555 | − | UCGUGGCGGGAAACCGC | 17 | 2699 |
| HSV1-UL19-904 | − | CGUGGCGGGAAACCGCC | 17 | 1290 |
| HSV1-UL19-2557 | − | CCAGCCCGUCCCCGUGU | 17 | 2700 |
| HSV1-UL19-2558 | − | CCAGGUGCCGCGGCGCG | 17 | 2701 |
| HSV1-UL19-912 | − | CAGGUGCCGCGGCGCGC | 17 | 1298 |
| HSV1-UL19-2560 | − | UGCCGCGGCGCGCAGGG | 17 | 2702 |
| HSV1-UL19-2561 | − | CAGGGAUGGACCACGGC | 17 | 2703 |
| HSV1-UL19-2562 | − | CGGCCAGGACGCCGUGU | 17 | 2704 |
| HSV1-UL19-2563 | − | CGCCGGCCCUGCAACCC | 17 | 2705 |
| HSV1-UL19-918 | − | GCCGGCCCUGCAACCCC | 17 | 1304 |
| HSV1-UL19-919 | − | CCGGCCCUGCAACCCCC | 17 | 1305 |
| HSV1-UL19-2566 | − | CCCCCGGGGACGCGCCG | 17 | 2706 |
| HSV1-UL19-921 | − | CCCCGGGGACGCGCCGC | 17 | 1307 |
| HSV1-UL19-922 | − | CCCGGGGACGCGCCGCC | 17 | 1308 |
| HSV1-UL19-2569 | − | CCGCCGGGGGCGUUUAC | 17 | 2707 |
| HSV1-UL19-925 | − | CGCCGGGGGCGUUUACG | 17 | 1311 |
| HSV1-UL19-926 | − | GCCGGGGGCGUUUACGC | 17 | 1312 |
| HSV1-UL19-927 | − | CCGGGGGCGUUUACGCG | 17 | 1313 |
| HSV1-UL19-2573 | − | GCGUUUACGCGGGGGAC | 17 | 2708 |
| HSV1-UL19-929 | − | CGUUUACGCGGGGGACA | 17 | 1315 |
| HSV1-UL19-2575 | − | UUUACGCGGGGGACAAG | 17 | 2709 |
| HSV1-UL19-930 | − | UUACGCGGGGGACAAGG | 17 | 1316 |
| HSV1-UL19-931 | − | UACGCGGGGGACAAGGA | 17 | 1317 |
| HSV1-UL19-932 | − | ACGCGGGGGACAAGGAG | 17 | 1318 |
| HSV1-UL19-2579 | − | UCAUGUACGACCACGGC | 17 | 2710 |
| HSV1-UL19-2580 | − | GGCCAGAGCGACCCGUC | 17 | 2711 |
| HSV1-UL19-2581 | − | GCGGCCACGGCCAACCC | 17 | 2712 |
| HSV1-UL19-2582 | − | GUCGCAGCGAUUUUCGU | 17 | 2713 |
| HSV1-UL19-941 | − | UCGCAGCGAUUUUCGUA | 17 | 1327 |
| HSV1-UL19-942 | − | CGCAGCGAUUUUCGUAC | 17 | 1328 |
| HSV1-UL19-2585 | − | CGGGGACCUGCUCUAUA | 17 | 2714 |
| HSV1-UL19-944 | − | GGGGACCUGCUCUAUAA | 17 | 1330 |
| HSV1-UL19-2587 | − | CGGGGCCUACCACCUCA | 17 | 2715 |
| HSV1-UL19-947 | − | GGGGCCUACCACCUCAA | 17 | 1333 |
| HSV1-UL19-2589 | − | CCGCCAAACAUCGCUGC | 17 | 2716 |
| HSV1-UL19-952 | − | CGCCAAACAUCGCUGCC | 17 | 1338 |
| HSV1-UL19-2591 | − | GCCUGGAGCGCCUGAUC | 17 | 2717 |
| HSV1-UL19-953 | − | CCUGGAGCGCCUGAUCG | 17 | 1339 |
| HSV1-UL19-2593 | − | AGCGCCUGAUCGUGGAG | 17 | 2718 |
| HSV1-UL19-2594 | − | UACAGUUUAAGCGCCCC | 17 | 2719 |
| HSV1-UL19-959 | − | ACAGUUUAAGCGCCCCC | 17 | 1345 |
| HSV1-UL19-2596 | − | GCGCCCCCCGGGGUGCC | 17 | 2720 |
| HSV1-UL19-2597 | − | CGGGGUGCCGCGAACUC | 17 | 2721 |
| HSV1-UL19-962 | − | GGGGUGCCGCGAACUCG | 17 | 1348 |
| HSV1-UL19-2599 | − | GGUGCCGCGAACUCGUG | 17 | 2722 |
| HSV1-UL19-2600 | − | ACCCGUGUGGCCUGUUU | 17 | 2723 |
| HSV1-UL19-965 | − | CCCGUGUGGCCUGUUUC | 17 | 1351 |
| HSV1-UL19-2602 | − | CCUCCGCAGUGCCCGCA | 17 | 2724 |
| HSV1-UL19-967 | − | CUCCGCAGUGCCCGCAA | 17 | 1353 |
| HSV1-UL19-968 | − | UCCGCAGUGCCCGCAAC | 17 | 1354 |
| HSV1-UL19-969 | − | CCGCAGUGCCCGCAACG | 17 | 1355 |
| HSV1-UL19-2606 | − | AACGGGGAAGCCCACGC | 17 | 2725 |
| HSV1-UL19-970 | − | ACGGGGAAGCCCACGCG | 17 | 1356 |
| HSV1-UL19-971 | − | CGGGGAAGCCCACGCGC | 17 | 1357 |
| HSV1-UL19-2609 | − | AUGACGCAUCCCCGCUC | 17 | 2726 |
| HSV1-UL19-972 | − | UGACGCAUCCCCGCUCA | 17 | 1358 |
| HSV1-UL19-2611 | + | UCAAACGCCCCGAGGACGGC | 20 | 2727 |
| HSV1-UL19-2612 | + | GUGCCGCGCUCAAACGCCCC | 20 | 2728 |
| HSV1-UL19-2613 | + | CCGUGCCGCGCUCAAACGCC | 20 | 2729 |
| HSV1-UL19-983 | + | GCAUCGGCAACAACAAAGCC | 20 | 1369 |
| HSV1-UL19-2615 | + | UGCAUCGGCAACAACAAAGC | 20 | 2730 |
| HSV1-UL19-2616 | + | AGCUCGGCGACCAGGGUCGC | 20 | 2731 |
| HSV1-UL19-2617 | + | CUUCGCUUUAGCUCGGCGAC | 20 | 2732 |
| HSV1-UL19-2618 | + | CCAGGAAAAAGCUCGUCUCG | 20 | 2733 |
| HSV1-UL19-995 | + | GGCGGUGGCCCGCCUUGCCC | 20 | 1381 |
| HSV1-UL19-2620 | + | CGGCGGUGGCCCGCCUUGCC | 20 | 2734 |
| HSV1-UL19-2621 | + | GCGUGGCCGUGGUGAGGUCC | 20 | 2735 |
| HSV1-UL19-2622 | + | CGGAGGGCUGCGUGGCCGUG | 20 | 2736 |
| HSV1-UL19-2623 | + | UCAGACGGGGCACGGCCACG | 20 | 2737 |
| HSV1-UL19-1006 | + | CGUCAGACGGGGCACGGCCA | 20 | 1392 |
| HSV1-UL19-2625 | + | GCGUCAGACGGGGCACGGCC | 20 | 2738 |
| HSV1-UL19-1010 | + | GCGUGUCGGCAUGCGUCAGA | 20 | 1396 |
| HSV1-UL19-2627 | + | CGCGUGUCGGCAUGCGUCAG | 20 | 2739 |
| HSV1-UL19-2628 | + | GUUUGAUCGGGGCGGUGGUG | 20 | 2740 |
| HSV1-UL19-1017 | + | UGCAGGAGGCGCUGUUUGAU | 20 | 1403 |
| HSV1-UL19-2630 | + | CUGCAGGAGGCGCUGUUUGA | 20 | 2741 |
| HSV1-UL19-1019 | + | CCACUUUCAGGAAGGACUGC | 20 | 1405 |
| HSV1-UL19-2632 | + | UCCACUUUCAGGAAGGACUG | 20 | 2742 |
| HSV1-UL19-2633 | + | CGGUGUCCUCCACUUUCAGG | 20 | 2743 |
| HSV1-UL19-1021 | + | CUUCGGUGUCCUCCACUUUC | 20 | 1407 |
| HSV1-UL19-2635 | + | GCUUCGGUGUCCUCCACUUU | 20 | 2744 |
| HSV1-UL19-2636 | + | GUGACCAGGUUGGCCCCGUU | 20 | 2745 |
| HSV1-UL19-2637 | + | UUCGCACGGCCUUGCCCAUC | 20 | 2746 |
| HSV1-UL19-2638 | + | CCAGCGUCUGCCGGUUCAGG | 20 | 2747 |
| HSV1-UL19-2639 | + | GCGUCGUCUGGGGGGCGCUC | 20 | 2748 |
| HSV1-UL19-1036 | + | GCGCGCACGCGCGUCGUCUG | 20 | 1422 |
| HSV1-UL19-1037 | + | CGCGCGCACGCGCGUCGUCU | 20 | 1423 |
| HSV1-UL19-1038 | + | CCGCGCGCACGCGCGUCGUC | 20 | 1424 |
| HSV1-UL19-2643 | + | UCCGCGCGCACGCGCGUCGU | 20 | 2749 |
| HSV1-UL19-2644 | + | GAAAGACCAGCUUCUCGCCG | 20 | 2750 |
| HSV1-UL19-2645 | + | AUGCGCUUCUCCAGGGCCUC | 20 | 2751 |
| HSV1-UL19-2646 | + | GCGGCGUAGAUGCGCUUCUC | 20 | 2752 |
| HSV1-UL19-1045 | + | AUGGCGCCCACCAGGGGGUA | 20 | 1431 |
| HSV1-UL19-1046 | + | CAUGGCGCCCACCAGGGGGU | 20 | 1432 |
| HSV1-UL19-2649 | + | CCAUGGCGCCCACCAGGGGG | 20 | 2753 |
| HSV1-UL19-1049 | + | CAGGUCCAUGGCGCCCACCA | 20 | 1435 |
| HSV1-UL19-1050 | + | UCAGGUCCAUGGCGCCCACC | 20 | 1436 |
| HSV1-UL19-2652 | + | GUCAGGUCCAUGGCGCCCAC | 20 | 2754 |
| HSV1-UL19-2653 | + | UGAACAGGCCCAGGGGCAGG | 20 | 2755 |
| HSV1-UL19-2654 | + | GGAUUGAACAGGCCCAGGGG | 20 | 2756 |
| HSV1-UL19-1056 | + | UGACCGGAUUGAACAGGCCC | 20 | 1442 |
| HSV1-UL19-2656 | + | AUGACCGGAUUGAACAGGCC | 20 | 2757 |
| HSV1-UL19-2657 | + | CAAACCGUUCCAUGACCGGA | 20 | 2758 |
| HSV1-UL19-2658 | + | CGCGGCAAACCGUUCCAUGA | 20 | 2759 |
| HSV1-UL19-1063 | + | GGAUCCGGGUGGCCGGGGGC | 20 | 1449 |
| HSV1-UL19-1064 | + | GGGAUCCGGGUGGCCGGGGG | 20 | 1450 |
| HSV1-UL19-2661 | + | GGGGAUCCGGGUGGCCGGGG | 20 | 2760 |
| HSV1-UL19-1067 | + | GCGCGGGGAUCCGGGUGGCC | 20 | 1453 |
| HSV1-UL19-1068 | + | GGCGCGGGGAUCCGGGUGGC | 20 | 1454 |
| HSV1-UL19-2664 | + | AGGCGCGGGGAUCCGGGUGG | 20 | 2761 |
| HSV1-UL19-2665 | + | GGGCGGGAAGGCGCGGGGAU | 20 | 2762 |
| HSV1-UL19-1073 | + | CUGGCGGGGCGGGAAGGCGC | 20 | 1459 |
| HSV1-UL19-1074 | + | GCUGGCGGGGCGGGAAGGCG | 20 | 1460 |
| HSV1-UL19-2668 | + | AGCUGGCGGGGCGGGAAGGC | 20 | 2763 |
| HSV1-UL19-1076 | + | AAAAAAACAGCUGGCGGGGC | 20 | 1462 |
| HSV1-UL19-1077 | + | CAAAAAAACAGCUGGCGGGG | 20 | 1463 |
| HSV1-UL19-2671 | + | CCAAAAAAACAGCUGGCGGG | 20 | 2764 |
| HSV1-UL19-1080 | + | UCCCCCAAAAAAACAGCUGG | 20 | 1466 |
| HSV1-UL19-2673 | + | UUCCCCCAAAAAAACAGCUG | 20 | 2765 |
| HSV1-UL19-2674 | + | ACGGUCCCGAUCGCGUGUUC | 20 | 2766 |
| HSV1-UL19-2675 | + | CGCGUCAACGUUCAUCAGCG | 20 | 2767 |
| HSV1-UL19-2676 | + | CGCCGCGUCAACGUUCAUCA | 20 | 2768 |
| HSV1-UL19-1093 | + | GUACGGAUUGGCGGCUUCGA | 20 | 1479 |
| HSV1-UL19-2678 | + | CGUACGGAUUGGCGGCUUCG | 20 | 2769 |
| HSV1-UL19-2679 | + | GGCCGCCACGUACGCCCCGU | 20 | 2770 |
| HSV1-UL19-1099 | + | AUGUCUGCGGCGGGGCCGGC | 20 | 1485 |
| HSV1-UL19-2681 | + | CAUGUCUGCGGCGGGGCCGG | 20 | 2771 |
| HSV1-UL19-1103 | + | CAGCUGCUGCAUGUCUGCGG | 20 | 1489 |
| HSV1-UL19-2683 | + | ACAGCUGCUGCAUGUCUGCG | 20 | 2772 |
| HSV1-UL19-2684 | + | GCGACCCAUCGGACCCGCCC | 20 | 2773 |
| HSV1-UL19-2685 | + | AUCUGGCCUUCCGCGACCCA | 20 | 2774 |
| HSV1-UL19-1113 | + | UCGGGCUGCAUAAACUGCUC | 20 | 1499 |
| HSV1-UL19-2687 | + | GUCGGGCUGCAUAAACUGCU | 20 | 2775 |
| HSV1-UL19-2688 | + | CAAGAGCCAGGUUGGCGUUG | 20 | 2776 |
| HSV1-UL19-2689 | + | UCGAACGCGGGGUGCAGCUC | 20 | 2777 |
| HSV1-UL19-1120 | + | CCCCACAAAGAAGUCGAACG | 20 | 1506 |
| HSV1-UL19-2691 | + | CCCCCACAAAGAAGUCGAAC | 20 | 2778 |
| HSV1-UL19-2692 | + | CGGCCACCCCCACAAAGAAG | 20 | 2779 |
| HSV1-UL19-2693 | + | CCACGUCGGCCACCCCCACA | 20 | 2780 |
| HSV1-UL19-1123 | + | AUCUCCCCCGGGCCGGCCGG | 20 | 1509 |
| HSV1-UL19-1124 | + | GAUCUCCCCCGGGCCGGCCG | 20 | 1510 |
| HSV1-UL19-1125 | + | GGAUCUCCCCCGGGCCGGCC | 20 | 1511 |
| HSV1-UL19-1126 | + | UGGAUCUCCCCCGGGCCGGC | 20 | 1512 |
| HSV1-UL19-2698 | + | CUGGAUCUCCCCCGGGCCGG | 20 | 2781 |
| HSV1-UL19-2699 | + | CCAGGUGGCCUGGAUCUCCC | 20 | 2782 |
| HSV1-UL19-2700 | + | UUCACCACGCGCCAGGUGGC | 20 | 2783 |
| HSV1-UL19-1135 | + | ACGCCGCCGGACAUAGCGCC | 20 | 1521 |
| HSV1-UL19-2702 | + | AACGCCGCCGGACAUAGCGC | 20 | 2784 |
| HSV1-UL19-2703 | + | CCGGGCGUCCCGGAACGCCG | 20 | 2785 |
| HSV1-UL19-1137 | + | GCUCCAGGCCCCGGGCGUCC | 20 | 1523 |
| HSV1-UL19-2705 | + | AGCUCCAGGCCCCGGGCGUC | 20 | 2786 |
| HSV1-UL19-2706 | + | CCCACCCCCAGCUCCAGGCC | 20 | 2787 |
| HSV1-UL19-1145 | + | CGAACGGCGGCGAUGGUGGC | 20 | 1531 |
| HSV1-UL19-1146 | + | GCGAACGGCGGCGAUGGUGG | 20 | 1532 |
| HSV1-UL19-2709 | + | CGCGAACGGCGGCGAUGGUG | 20 | 2788 |
| HSV1-UL19-2710 | + | UUGCGGUCGUCGAACGCCCC | 20 | 2789 |
| HSV1-UL19-2711 | + | CCGCCGGGUAGUUGCGGUCG | 20 | 2790 |
| HSV1-UL19-2712 | + | CUGCAGCAGGUAAAACACCG | 20 | 2791 |
| HSV1-UL19-2713 | + | CCACGAGCCGGGCCAGGGCG | 20 | 2792 |
| HSV1-UL19-2714 | + | CACUGGACCACGAGCCGGGC | 20 | 2793 |
| HSV1-UL19-2715 | + | GUGAUGCACUGGACCACGAG | 20 | 2794 |
| HSV1-UL19-2716 | + | AGCUGGUGAUGCACUGGACC | 20 | 2795 |
| HSV1-UL19-2717 | + | UUCCAGUAGCUGGUGAUGCA | 20 | 2796 |
| HSV1-UL19-2718 | + | AGACCAGCGAGUAGUCGUUC | 20 | 2797 |
| HSV1-UL19-2719 | + | GGUCACGACGUACGAGACCA | 20 | 2798 |
| HSV1-UL19-2720 | + | CCCGAGGUAGGUCACGACGU | 20 | 2799 |
| HSV1-UL19-2721 | + | ACUCCUCGGGAAGGUCUCCC | 20 | 2800 |
| HSV1-UL19-1166 | + | UACACGGCCAUGCACUCCUC | 20 | 1552 |
| HSV1-UL19-1167 | + | GUACACGGCCAUGCACUCCU | 20 | 1553 |
| HSV1-UL19-2724 | + | GGUACACGGCCAUGCACUCC | 20 | 2801 |
| HSV1-UL19-2725 | + | AAGUCAUCAACCAGCUGGGC | 20 | 2802 |
| HSV1-UL19-2726 | + | AGGGUAAAGUCAUCAACCAG | 20 | 2803 |
| HSV1-UL19-2727 | + | CCGCCCAGCUCCGGGCCGGU | 20 | 2804 |
| HSV1-UL19-2728 | + | UUGCGCCUGCCCGCCCAGCU | 20 | 2805 |
| HSV1-UL19-2729 | + | CACGAGGGGUGGCAGCAGCG | 20 | 2806 |
| HSV1-UL19-1187 | + | GGGCGUCACAGUCCCACACG | 20 | 1573 |
| HSV1-UL19-2731 | + | AGGGCGUCACAGUCCCACAC | 20 | 2807 |
| HSV1-UL19-2732 | + | UCAGGGCGUCACAGUCCCAC | 20 | 2808 |
| HSV1-UL19-2733 | + | UCCAGGGCCGCGCGCCGCAU | 20 | 2809 |
| HSV1-UL19-2734 | + | CGGCAGUCGCGAUGGCGGUC | 20 | 2810 |
| HSV1-UL19-1198 | + | GUUACAUGCCGCCGCGUACA | 20 | 1584 |
| HSV1-UL19-2736 | + | CGUUACAUGCCGCCGCGUAC | 20 | 2811 |
| HSV1-UL19-2737 | + | GCAGCUGGCCGUCGUUGCGG | 20 | 2812 |
| HSV1-UL19-2738 | + | GCGGCGUCCGCGGCUCGGGC | 20 | 2813 |
| HSV1-UL19-2739 | + | UCAUCCGCGGCGUCCGCGGC | 20 | 2814 |
| HSV1-UL19-2740 | + | AGCAGCGGCCCCGCGAGAAG | 20 | 2815 |
| HSV1-UL19-2741 | + | CGGUGCAGCAGCGGCCCCGC | 20 | 2816 |
| HSV1-UL19-2742 | + | CGCGGUGCAGCAGCGGCCCC | 20 | 2817 |
| HSV1-UL19-2743 | + | GUGGCGUAUACGCGGUCGAA | 20 | 2818 |
| HSV1-UL19-2744 | + | GCAGGGUGGCGUAUACGCGG | 20 | 2819 |
| HSV1-UL19-2745 | + | UCCGGGACCACCAUGUUCUG | 20 | 2820 |
| HSV1-UL19-1223 | + | UCCUCGCCGGGGGCGAUCUC | 20 | 1609 |
| HSV1-UL19-2747 | + | CUCCUCGCCGGGGGCGAUCU | 20 | 2821 |
| HSV1-UL19-1226 | + | UCGCUGGGGCACUCCUCGCC | 20 | 1612 |
| HSV1-UL19-1227 | + | GUCGCUGGGGCACUCCUCGC | 20 | 1613 |
| HSV1-UL19-2750 | + | GGUCGCUGGGGCACUCCUCG | 20 | 2822 |
| HSV1-UL19-1230 | + | GGGGUCCGUCACGGGGUCGC | 20 | 1616 |
| HSV1-UL19-2752 | + | CGGGGUCCGUCACGGGGUCG | 20 | 2823 |
| HSV1-UL19-1233 | + | GGGGUGCGCGGGGUCCGUCA | 20 | 1619 |
| HSV1-UL19-2754 | + | GGGGGUGCGCGGGGUCCGUC | 20 | 2824 |
| HSV1-UL19-1236 | + | GGCCGGGUGCAGGGGGUGCG | 20 | 1622 |
| HSV1-UL19-2756 | + | UGGCCGGGUGCAGGGGGUGC | 20 | 2825 |
| HSV1-UL19-1239 | + | CACCAGAUUGGCCGGGUGCA | 20 | 1625 |
| HSV1-UL19-1240 | + | CCACCAGAUUGGCCGGGUGC | 20 | 1626 |
| HSV1-UL19-2759 | + | GCCACCAGAUUGGCCGGGUG | 20 | 2826 |
| HSV1-UL19-2760 | + | CGUGUUGGCCACCAGAUUGG | 20 | 2827 |
| HSV1-UL19-2761 | + | UCCACUACCACGCGCCCGUU | 20 | 2828 |
| HSV1-UL19-2762 | + | GCACCUGCAGCGUGAGCAUG | 20 | 2829 |
| HSV1-UL19-2763 | + | UGUGGGCCAGCACCUGCAGC | 20 | 2830 |
| HSV1-UL19-2764 | + | GUUGUGCGUUCGGCCAUGUU | 20 | 2831 |
| HSV1-UL19-2765 | + | CCGCGUCGGGCGCCGCCGAG | 20 | 2832 |
| HSV1-UL19-2766 | + | GGCGCCCGCGUCGGGCGCCG | 20 | 2833 |
| HSV1-UL19-2767 | + | ACGCGGUGUUGGCGCCCGCG | 20 | 2834 |
| HSV1-UL19-2768 | + | CGAAUAUGCGCAUGUUGGUG | 20 | 2835 |
| HSV1-UL19-2769 | + | UUCCGGCGUGCAACGCCCCG | 20 | 2836 |
| HSV1-UL19-2770 | + | AGAUGCUGGGGGGCCAUCAG | 20 | 2837 |
| HSV1-UL19-1263 | + | AUGGUAUGGUCCAGAUGCUG | 20 | 1649 |
| HSV1-UL19-1264 | + | GAUGGUAUGGUCCAGAUGCU | 20 | 1650 |
| HSV1-UL19-1265 | + | GGAUGGUAUGGUCCAGAUGC | 20 | 1651 |
| HSV1-UL19-2774 | + | UGGAUGGUAUGGUCCAGAUG | 20 | 2838 |
| HSV1-UL19-2775 | + | GGGUAAAAAUAGUCGCCAUU | 20 | 2839 |
| HSV1-UL19-1271 | + | GAACAGCGCGUGGACGGGGA | 20 | 1657 |
| HSV1-UL19-1272 | + | CGAACAGCGCGUGGACGGGG | 20 | 1658 |
| HSV1-UL19-2778 | + | GCGAACAGCGCGUGGACGGG | 20 | 2840 |
| HSV1-UL19-1273 | + | CGGCGAACAGCGCGUGGACG | 20 | 1659 |
| HSV1-UL19-1274 | + | CCGGCGAACAGCGCGUGGAC | 20 | 1660 |
| HSV1-UL19-1275 | + | CCCGGCGAACAGCGCGUGGA | 20 | 1661 |
| HSV1-UL19-2782 | + | CCCCGGCGAACAGCGCGUGG | 20 | 2841 |
| HSV1-UL19-2783 | + | UCGGCCCCGGCGAACAGCGC | 20 | 2842 |
| HSV1-UL19-2784 | + | UCGCCACGUGGUCGGCCCCG | 20 | 2843 |
| HSV1-UL19-2785 | + | CGCGCAGGGCCGGGGGGAAA | 20 | 2844 |
| HSV1-UL19-1282 | + | ACAGGUCGCGCAGGGCCGGG | 20 | 1668 |
| HSV1-UL19-1283 | + | GACAGGUCGCGCAGGGCCGG | 20 | 1669 |
| HSV1-UL19-1284 | + | CGACAGGUCGCGCAGGGCCG | 20 | 1670 |
| HSV1-UL19-1285 | + | GCGACAGGUCGCGCAGGGCC | 20 | 1671 |
| HSV1-UL19-1286 | + | CGCGACAGGUCGCGCAGGGC | 20 | 1672 |
| HSV1-UL19-2791 | + | CCGCGACAGGUCGCGCAGGG | 20 | 2845 |
| HSV1-UL19-2792 | + | ACCUGCCGCGACAGGUCGCG | 20 | 2846 |
| HSV1-UL19-1291 | + | CCCAGAGCCGGGGGGACCAG | 20 | 1677 |
| HSV1-UL19-1292 | + | CCCCAGAGCCGGGGGGACCA | 20 | 1678 |
| HSV1-UL19-1293 | + | CCCCCAGAGCCGGGGGGACC | 20 | 1679 |
| HSV1-UL19-2796 | + | GCCCCCAGAGCCGGGGGGAC | 20 | 2847 |
| HSV1-UL19-1295 | + | UAGUUGGCCCCCAGAGCCGG | 20 | 1681 |
| HSV1-UL19-1296 | + | GUAGUUGGCCCCCAGAGCCG | 20 | 1682 |
| HSV1-UL19-1297 | + | AGUAGUUGGCCCCCAGAGCC | 20 | 1683 |
| HSV1-UL19-1298 | + | AAGUAGUUGGCCCCCAGAGC | 20 | 1684 |
| HSV1-UL19-2801 | + | AAAGUAGUUGGCCCCCAGAG | 20 | 2848 |
| HSV1-UL19-2802 | + | GACGAAAAGUAGUUGGCCCC | 20 | 2849 |
| HSV1-UL19-2803 | + | CACGACGGGCUGUCGGAUCG | 20 | 2850 |
| HSV1-UL19-2804 | + | ACGUGCUGCACGACGGGCUG | 20 | 2851 |
| HSV1-UL19-2805 | + | UCUCGCGGACGUGCUGCACG | 20 | 2852 |
| HSV1-UL19-2806 | + | UUCUCCCCGGCCGCGCUCUC | 20 | 2853 |
| HSV1-UL19-2807 | + | UGAUCUUGAAGUACCCCGCC | 20 | 2854 |
| HSV1-UL19-2808 | + | GCAAGGCCACGGGACUGAUC | 20 | 2855 |
| HSV1-UL19-1307 | + | GAGCUGAUGAUGCAAGGCCA | 20 | 1693 |
| HSV1-UL19-2810 | + | UGAGCUGAUGAUGCAAGGCC | 20 | 2856 |
| HSV1-UL19-2811 | + | ACCCGGGAUGGAGGCCCGUC | 20 | 2857 |
| HSV1-UL19-1310 | + | CGGUGAACCCAAACCCGGGA | 20 | 1696 |
| HSV1-UL19-2813 | + | ACGGUGAACCCAAACCCGGG | 20 | 2858 |
| HSV1-UL19-1312 | + | GACGACGGUGAACCCAAACC | 20 | 1698 |
| HSV1-UL19-2815 | + | GGACGACGGUGAACCCAAAC | 20 | 2859 |
| HSV1-UL19-2816 | + | AGCGGUCCUGUCGGACGACG | 20 | 2860 |
| HSV1-UL19-2817 | + | UCAGUCACAAAGCGGUCCUG | 20 | 2861 |
| HSV1-UL19-2818 | + | ACGCCUCCGACGCGCGCUCC | 20 | 2862 |
| HSV1-UL19-2819 | + | GUACGCCUCCGACGCGCGCU | 20 | 2863 |
| HSV1-UL19-1316 | + | GGGCCACCUGGAGCUGGCCC | 20 | 1702 |
| HSV1-UL19-2821 | + | CGGGCCACCUGGAGCUGGCC | 20 | 2864 |
| HSV1-UL19-1318 | + | CAGUUUCGUGCCGGGCCACC | 20 | 1704 |
| HSV1-UL19-2823 | + | CCAGUUUCGUGCCGGGCCAC | 20 | 2865 |
| HSV1-UL19-2824 | + | ACCCCCCCGCCAGUUUCGUG | 20 | 2866 |
| HSV1-UL19-2825 | + | UCGCGCGCGGCUGGGUGAGC | 20 | 2867 |
| HSV1-UL19-2826 | + | CCACGUUCGCGCGCGGCUGG | 20 | 2868 |
| HSV1-UL19-2827 | + | AGGUCCACGUUCGCGCGCGG | 20 | 2869 |
| HSV1-UL19-2828 | + | CCCAUGUCGGUGACGGGGUU | 20 | 2870 |
| HSV1-UL19-1332 | + | AAGGUUGCCCAUGUCGGUGA | 20 | 1718 |
| HSV1-UL19-2830 | + | GAAGGUUGCCCAUGUCGGUG | 20 | 2871 |
| HSV1-UL19-1335 | + | CGGCCCAGGUAAAAGUUUUG | 20 | 1721 |
| HSV1-UL19-1336 | + | GCGGCCCAGGUAAAAGUUUU | 20 | 1722 |
| HSV1-UL19-1337 | + | CGCGGCCCAGGUAAAAGUUU | 20 | 1723 |
| HSV1-UL19-2834 | + | CCGCGGCCCAGGUAAAAGUU | 20 | 2872 |
| HSV1-UL19-1340 | + | GCCGCGUUGUCCAGGAGAGG | 20 | 1726 |
| HSV1-UL19-1341 | + | UGCCGCGUUGUCCAGGAGAG | 20 | 1727 |
| HSV1-UL19-1342 | + | CUGCCGCGUUGUCCAGGAGA | 20 | 1728 |
| HSV1-UL19-1343 | + | GCUGCCGCGUUGUCCAGGAG | 20 | 1729 |
| HSV1-UL19-2839 | + | GGCUGCCGCGUUGUCCAGGA | 20 | 2873 |
| HSV1-UL19-2840 | + | ACGGCUGCCGCGUUGUCCAG | 20 | 2874 |
| HSV1-UL19-1344 | + | ACACGGCUGCCGCGUUGUCC | 20 | 1730 |
| HSV1-UL19-2842 | + | UACACGGCUGCCGCGUUGUC | 20 | 2875 |
| HSV1-UL19-2843 | + | CACCCGAACACGGGGACGGG | 20 | 2876 |
| HSV1-UL19-2844 | + | GGGCGCACCCGAACACGGGG | 20 | 2877 |
| HSV1-UL19-1355 | + | ACCUGGGCGCACCCGAACAC | 20 | 1741 |
| HSV1-UL19-1356 | + | CACCUGGGCGCACCCGAACA | 20 | 1742 |
| HSV1-UL19-2847 | + | GCACCUGGGCGCACCCGAAC | 20 | 2878 |
| HSV1-UL19-2848 | + | GCCGCGGCACCUGGGCGCAC | 20 | 2879 |
| HSV1-UL19-2849 | + | AUCCCUGCGCGCCGCGGCAC | 20 | 2880 |
| HSV1-UL19-1366 | + | UAGUUGACGUCGGUCGACAC | 20 | 1752 |
| HSV1-UL19-1367 | + | GUAGUUGACGUCGGUCGACA | 20 | 1753 |
| HSV1-UL19-2852 | + | AGUAGUUGACGUCGGUCGAC | 20 | 2881 |
| HSV1-UL19-1369 | + | CCCGGGGGUUGCAGGGCCGG | 20 | 1755 |
| HSV1-UL19-2854 | + | CCCCGGGGGUUGCAGGGCCG | 20 | 2882 |
| HSV1-UL19-2855 | + | CGGCGCGUCCCCGGGGGUUG | 20 | 2883 |
| HSV1-UL19-1375 | + | GCCCCCGGCGGCGCGUCCCC | 20 | 1761 |
| HSV1-UL19-1376 | + | CGCCCCCGGCGGCGCGUCCC | 20 | 1762 |
| HSV1-UL19-2858 | + | ACGCCCCCGGCGGCGCGUCC | 20 | 2884 |
| HSV1-UL19-2859 | + | CUCUGGCCGUGGUCGUACAU | 20 | 2885 |
| HSV1-UL19-2860 | + | CGCUCUGGCCGUGGUCGUAC | 20 | 2886 |
| HSV1-UL19-2861 | + | CGUGGCCGCGAAGGCCCGGG | 20 | 2887 |
| HSV1-UL19-1387 | + | UGGCCGUGGCCGCGAAGGCC | 20 | 1773 |
| HSV1-UL19-2863 | + | UUGGCCGUGGCCGCGAAGGC | 20 | 2888 |
| HSV1-UL19-2864 | + | CCCACGGGUUGGCCGUGGCC | 20 | 2889 |
| HSV1-UL19-2865 | + | CGAAAAUCGCUGCGACGCCC | 20 | 2890 |
| HSV1-UL19-2866 | + | GUUAUAGAGCAGGUCCCCGU | 20 | 2891 |
| HSV1-UL19-2867 | + | UGAGGUGGUAGGCCCCGUUA | 20 | 2892 |
| HSV1-UL19-2868 | + | UGAGCACCGGCGAGGCCCCG | 20 | 2893 |
| HSV1-UL19-2869 | + | AAAGCAGGGGCUGAGCACCG | 20 | 2894 |
| HSV1-UL19-2870 | + | UAAAGAACUUAAAGCAGGGG | 20 | 2895 |
| HSV1-UL19-1401 | + | CGACGUAAAGAACUUAAAGC | 20 | 1787 |
| HSV1-UL19-2872 | + | CCGACGUAAAGAACUUAAAG | 20 | 2896 |
| HSV1-UL19-2873 | + | CGGCGAUGUCGGCCGACGUA | 20 | 2897 |
| HSV1-UL19-2874 | + | GGCGGUGGCCGUGGACACCG | 20 | 2898 |
| HSV1-UL19-2875 | + | CGUCGCUGGCGGCGGUGGCC | 20 | 2899 |
| HSV1-UL19-1414 | + | CACGAGUUCGCGGCACCCCG | 20 | 1800 |
| HSV1-UL19-1415 | + | CCACGAGUUCGCGGCACCCC | 20 | 1801 |
| HSV1-UL19-1416 | + | UCCACGAGUUCGCGGCACCC | 20 | 1802 |
| HSV1-UL19-2879 | + | CUCCACGAGUUCGCGGCACC | 20 | 2900 |
| HSV1-UL19-2880 | + | ACAGGCCACACGGGUCCUCC | 20 | 2901 |
| HSV1-UL19-2881 | + | GGCCUCCUGAAACAGGCCAC | 20 | 2902 |
| HSV1-UL19-2882 | + | CAGGUGAGCGGGUAGGCCUC | 20 | 2903 |
| HSV1-UL19-2883 | + | GGGGUCGCUGGCGCAGGUGA | 20 | 2904 |
| HSV1-UL19-2884 | + | GGGCGGGGUCGCUGGCGCAG | 20 | 2905 |
| HSV1-UL19-1428 | + | GCGGGCACUGCGGAGGAGGG | 20 | 1814 |
| HSV1-UL19-2886 | + | UGCGGGCACUGCGGAGGAGG | 20 | 2906 |
| HSV1-UL19-2887 | + | CCGUUGCGGGCACUGCGGAG | 20 | 2907 |
| HSV1-UL19-1431 | + | CCCCGUUGCGGGCACUGCGG | 20 | 1817 |
| HSV1-UL19-2889 | + | UCCCCGUUGCGGGCACUGCG | 20 | 2908 |
| HSV1-UL19-1432 | + | CUUCCCCGUUGCGGGCACUG | 20 | 1818 |
| HSV1-UL19-2891 | + | GCUUCCCCGUUGCGGGCACU | 20 | 2909 |
| HSV1-UL19-2892 | + | CGCGCGUGGGCUUCCCCGUU | 20 | 2910 |
| HSV1-UL19-2893 | + | GCGAAGUGGGUCUCCCGCGC | 20 | 2911 |
| HSV1-UL19-2894 | + | UAGACGAGAUACUGCGCGAA | 20 | 2912 |
| HSV1-UL19-2895 | + | CGUCAUAGACGAGAUACUGC | 20 | 2913 |
| HSV1-UL19-2896 | + | UGAGCGGGGAUGCGUCAUAG | 20 | 2914 |
| HSV1-UL19-1440 | + | ACAGAGCCAGUCCCUUGAGC | 20 | 1826 |
| HSV1-UL19-1441 | + | UACAGAGCCAGUCCCUUGAG | 20 | 1827 |
| HSV1-UL19-2899 | + | UUACAGAGCCAGUCCCUUGA | 20 | 2915 |
| HSV1-UL19-2900 | + | AACGCCCCGAGGACGGC | 17 | 2916 |
| HSV1-UL19-2901 | + | CCGCGCUCAAACGCCCC | 17 | 2917 |
| HSV1-UL19-2902 | + | UGCCGCGCUCAAACGCC | 17 | 2918 |
| HSV1-UL19-1450 | + | UCGGCAACAACAAAGCC | 17 | 1836 |
| HSV1-UL19-2904 | + | AUCGGCAACAACAAAGC | 17 | 2919 |
| HSV1-UL19-2905 | + | UCGGCGACCAGGGUCGC | 17 | 2920 |
| HSV1-UL19-2906 | + | CGCUUUAGCUCGGCGAC | 17 | 2921 |
| HSV1-UL19-2907 | + | GGAAAAAGCUCGUCUCG | 17 | 2922 |
| HSV1-UL19-1462 | + | GGUGGCCCGCCUUGCCC | 17 | 1848 |
| HSV1-UL19-2909 | + | CGGUGGCCCGCCUUGCC | 17 | 2923 |
| HSV1-UL19-2910 | + | UGGCCGUGGUGAGGUCC | 17 | 2924 |
| HSV1-UL19-2911 | + | AGGGCUGCGUGGCCGUG | 17 | 2925 |
| HSV1-UL19-2912 | + | GACGGGGCACGGCCACG | 17 | 2926 |
| HSV1-UL19-1473 | + | CAGACGGGGCACGGCCA | 17 | 1859 |
| HSV1-UL19-2914 | + | UCAGACGGGGCACGGCC | 17 | 2927 |
| HSV1-UL19-1477 | + | UGUCGGCAUGCGUCAGA | 17 | 1863 |
| HSV1-UL19-2916 | + | GUGUCGGCAUGCGUCAG | 17 | 2928 |
| HSV1-UL19-2917 | + | UGAUCGGGGCGGUGGUG | 17 | 2929 |
| HSV1-UL19-1484 | + | AGGAGGCGCUGUUUGAU | 17 | 1870 |
| HSV1-UL19-2919 | + | CAGGAGGCGCUGUUUGA | 17 | 2930 |
| HSV1-UL19-1486 | + | CUUUCAGGAAGGACUGC | 17 | 1872 |
| HSV1-UL19-2921 | + | ACUUUCAGGAAGGACUG | 17 | 2931 |
| HSV1-UL19-2922 | + | UGUCCUCCACUUUCAGG | 17 | 2932 |
| HSV1-UL19-1488 | + | CGGUGUCCUCCACUUUC | 17 | 1874 |
| HSV1-UL19-2924 | + | UCGGUGUCCUCCACUUU | 17 | 2933 |
| HSV1-UL19-2925 | + | ACCAGGUUGGCCCCGUU | 17 | 2934 |
| HSV1-UL19-2926 | + | GCACGGCCUUGCCCAUC | 17 | 2935 |
| HSV1-UL19-2927 | + | GCGUCUGCCGGUUCAGG | 17 | 2936 |
| HSV1-UL19-2928 | + | UCGUCUGGGGGGCGCUC | 17 | 2937 |
| HSV1-UL19-1503 | + | CGCACGCGCGUCGUCUG | 17 | 1889 |
| HSV1-UL19-1504 | + | GCGCACGCGCGUCGUCU | 17 | 1890 |
| HSV1-UL19-1505 | + | CGCGCACGCGCGUCGUC | 17 | 1891 |
| HSV1-UL19-2932 | + | GCGCGCACGCGCGUCGU | 17 | 2938 |
| HSV1-UL19-2933 | + | AGACCAGCUUCUCGCCG | 17 | 2939 |
| HSV1-UL19-2934 | + | CGCUUCUCCAGGGCCUC | 17 | 2940 |
| HSV1-UL19-2935 | + | GCGUAGAUGCGCUUCUC | 17 | 2941 |
| HSV1-UL19-1512 | + | GCGCCCACCAGGGGGUA | 17 | 1898 |
| HSV1-UL19-1513 | + | GGCGCCCACCAGGGGGU | 17 | 1899 |
| HSV1-UL19-2938 | + | UGGCGCCCACCAGGGGG | 17 | 2942 |
| HSV1-UL19-1516 | + | GUCCAUGGCGCCCACCA | 17 | 1902 |
| HSV1-UL19-1517 | + | GGUCCAUGGCGCCCACC | 17 | 1903 |
| HSV1-UL19-2941 | + | AGGUCCAUGGCGCCCAC | 17 | 2943 |
| HSV1-UL19-2942 | + | ACAGGCCCAGGGGCAGG | 17 | 2944 |
| HSV1-UL19-2943 | + | UUGAACAGGCCCAGGGG | 17 | 2945 |
| HSV1-UL19-1523 | + | CCGGAUUGAACAGGCCC | 17 | 1909 |
| HSV1-UL19-2945 | + | ACCGGAUUGAACAGGCC | 17 | 2946 |
| HSV1-UL19-2946 | + | ACCGUUCCAUGACCGGA | 17 | 2947 |
| HSV1-UL19-2947 | + | GGCAAACCGUUCCAUGA | 17 | 2948 |
| HSV1-UL19-1530 | + | UCCGGGUGGCCGGGGGC | 17 | 1916 |
| HSV1-UL19-1531 | + | AUCCGGGUGGCCGGGGG | 17 | 1917 |
| HSV1-UL19-2950 | + | GAUCCGGGUGGCCGGGG | 17 | 2949 |
| HSV1-UL19-1534 | + | CGGGGAUCCGGGUGGCC | 17 | 1920 |
| HSV1-UL19-1535 | + | GCGGGGAUCCGGGUGGC | 17 | 1921 |
| HSV1-UL19-2953 | + | CGCGGGGAUCCGGGUGG | 17 | 2950 |
| HSV1-UL19-2954 | + | CGGGAAGGCGCGGGGAU | 17 | 2951 |
| HSV1-UL19-1540 | + | GCGGGGCGGGAAGGCGC | 17 | 1926 |
| HSV1-UL19-1541 | + | GGCGGGGCGGGAAGGCG | 17 | 1927 |
| HSV1-UL19-2957 | + | UGGCGGGGCGGGAAGGC | 17 | 2952 |
| HSV1-UL19-1543 | + | AAAACAGCUGGCGGGGC | 17 | 1929 |
| HSV1-UL19-1544 | + | AAAAACAGCUGGCGGGG | 17 | 1930 |
| HSV1-UL19-2960 | + | AAAAAACAGCUGGCGGG | 17 | 2953 |
| HSV1-UL19-1547 | + | CCCAAAAAAACAGCUGG | 17 | 1933 |
| HSV1-UL19-2962 | + | CCCCAAAAAAACAGCUG | 17 | 2954 |
| HSV1-UL19-2963 | + | GUCCCGAUCGCGUGUUC | 17 | 2955 |
| HSV1-UL19-2964 | + | GUCAACGUUCAUCAGCG | 17 | 2956 |
| HSV1-UL19-2965 | + | CGCGUCAACGUUCAUCA | 17 | 2957 |
| HSV1-UL19-1560 | + | CGGAUUGGCGGCUUCGA | 17 | 1946 |
| HSV1-UL19-2967 | + | ACGGAUUGGCGGCUUCG | 17 | 2958 |
| HSV1-UL19-2968 | + | CGCCACGUACGCCCCGU | 17 | 2959 |
| HSV1-UL19-1566 | + | UCUGCGGCGGGGCCGGC | 17 | 1952 |
| HSV1-UL19-2970 | + | GUCUGCGGCGGGGCCGG | 17 | 2960 |
| HSV1-UL19-1570 | + | CUGCUGCAUGUCUGCGG | 17 | 1956 |
| HSV1-UL19-2972 | + | GCUGCUGCAUGUCUGCG | 17 | 2961 |
| HSV1-UL19-2973 | + | ACCCAUCGGACCCGCCC | 17 | 2962 |
| HSV1-UL19-2974 | + | UGGCCUUCCGCGACCCA | 17 | 2963 |
| HSV1-UL19-1580 | + | GGCUGCAUAAACUGCUC | 17 | 1966 |
| HSV1-UL19-2976 | + | GGGCUGCAUAAACUGCU | 17 | 2964 |
| HSV1-UL19-2977 | + | GAGCCAGGUUGGCGUUG | 17 | 2965 |
| HSV1-UL19-2978 | + | AACGCGGGGUGCAGCUC | 17 | 2966 |
| HSV1-UL19-1587 | + | CACAAAGAAGUCGAACG | 17 | 1973 |
| HSV1-UL19-2980 | + | CCACAAAGAAGUCGAAC | 17 | 2967 |
| HSV1-UL19-2981 | + | CCACCCCCACAAAGAAG | 17 | 2968 |
| HSV1-UL19-2982 | + | CGUCGGCCACCCCCACA | 17 | 2969 |
| HSV1-UL19-1590 | + | UCCCCCGGGCCGGCCGG | 17 | 1976 |
| HSV1-UL19-1591 | + | CUCCCCCGGGCCGGCCG | 17 | 1977 |
| HSV1-UL19-1592 | + | UCUCCCCCGGGCCGGCC | 17 | 1978 |
| HSV1-UL19-1593 | + | AUCUCCCCCGGGCCGGC | 17 | 1979 |
| HSV1-UL19-2987 | + | GAUCUCCCCCGGGCCGG | 17 | 2970 |
| HSV1-UL19-2988 | + | GGUGGCCUGGAUCUCCC | 17 | 2971 |
| HSV1-UL19-2989 | + | ACCACGCGCCAGGUGGC | 17 | 2972 |
| HSV1-UL19-1602 | + | CCGCCGGACAUAGCGCC | 17 | 1988 |
| HSV1-UL19-2991 | + | GCCGCCGGACAUAGCGC | 17 | 2973 |
| HSV1-UL19-2992 | + | GGCGUCCCGGAACGCCG | 17 | 2974 |
| HSV1-UL19-1604 | + | CCAGGCCCCGGGCGUCC | 17 | 1990 |
| HSV1-UL19-2994 | + | UCCAGGCCCCGGGCGUC | 17 | 2975 |
| HSV1-UL19-2995 | + | ACCCCCAGCUCCAGGCC | 17 | 2976 |
| HSV1-UL19-1612 | + | ACGGCGGCGAUGGUGGC | 17 | 1998 |
| HSV1-UL19-1613 | + | AACGGCGGCGAUGGUGG | 17 | 1999 |
| HSV1-UL19-2998 | + | GAACGGCGGCGAUGGUG | 17 | 2977 |
| HSV1-UL19-2999 | + | CGGUCGUCGAACGCCCC | 17 | 2978 |
| HSV1-UL19-3000 | + | CCGGGUAGUUGCGGUCG | 17 | 2979 |
| HSV1-UL19-3001 | + | CAGCAGGUAAAACACCG | 17 | 2980 |
| HSV1-UL19-3002 | + | CGAGCCGGGCCAGGGCG | 17 | 2981 |
| HSV1-UL19-3003 | + | UGGACCACGAGCCGGGC | 17 | 2982 |
| HSV1-UL19-3004 | + | AUGCACUGGACCACGAG | 17 | 2983 |
| HSV1-UL19-3005 | + | UGGUGAUGCACUGGACC | 17 | 2984 |
| HSV1-UL19-3006 | + | CAGUAGCUGGUGAUGCA | 17 | 2985 |
| HSV1-UL19-3007 | + | CCAGCGAGUAGUCGUUC | 17 | 2986 |
| HSV1-UL19-3008 | + | CACGACGUACGAGACCA | 17 | 2987 |
| HSV1-UL19-3009 | + | GAGGUAGGUCACGACGU | 17 | 2988 |
| HSV1-UL19-3010 | + | CCUCGGGAAGGUCUCCC | 17 | 2989 |
| HSV1-UL19-1633 | + | ACGGCCAUGCACUCCUC | 17 | 2019 |
| HSV1-UL19-1634 | + | CACGGCCAUGCACUCCU | 17 | 2020 |
| HSV1-UL19-3013 | + | ACACGGCCAUGCACUCC | 17 | 2990 |
| HSV1-UL19-3014 | + | UCAUCAACCAGCUGGGC | 17 | 2991 |
| HSV1-UL19-3015 | + | GUAAAGUCAUCAACCAG | 17 | 2992 |
| HSV1-UL19-3016 | + | CCCAGCUCCGGGCCGGU | 17 | 2993 |
| HSV1-UL19-3017 | + | CGCCUGCCCGCCCAGCU | 17 | 2994 |
| HSV1-UL19-3018 | + | GAGGGGUGGCAGCAGCG | 17 | 2995 |
| HSV1-UL19-1654 | + | CGUCACAGUCCCACACG | 17 | 2040 |
| HSV1-UL19-3020 | + | GCGUCACAGUCCCACAC | 17 | 2996 |
| HSV1-UL19-3021 | + | GGGCGUCACAGUCCCAC | 17 | 2997 |
| HSV1-UL19-3022 | + | AGGGCCGCGCGCCGCAU | 17 | 2998 |
| HSV1-UL19-3023 | + | CAGUCGCGAUGGCGGUC | 17 | 2999 |
| HSV1-UL19-1665 | + | ACAUGCCGCCGCGUACA | 17 | 2051 |
| HSV1-UL19-3025 | + | UACAUGCCGCCGCGUAC | 17 | 3000 |
| HSV1-UL19-3026 | + | GCUGGCCGUCGUUGCGG | 17 | 3001 |
| HSV1-UL19-3027 | + | GCGUCCGCGGCUCGGGC | 17 | 3002 |
| HSV1-UL19-3028 | + | UCCGCGGCGUCCGCGGC | 17 | 3003 |
| HSV1-UL19-3029 | + | AGCGGCCCCGCGAGAAG | 17 | 3004 |
| HSV1-UL19-3030 | + | UGCAGCAGCGGCCCCGC | 17 | 3005 |
| HSV1-UL19-3031 | + | GGUGCAGCAGCGGCCCC | 17 | 3006 |
| HSV1-UL19-3032 | + | GCGUAUACGCGGUCGAA | 17 | 3007 |
| HSV1-UL19-3033 | + | GGGUGGCGUAUACGCGG | 17 | 3008 |
| HSV1-UL19-3034 | + | GGGACCACCAUGUUCUG | 17 | 3009 |
| HSV1-UL19-1690 | + | UCGCCGGGGGCGAUCUC | 17 | 2076 |
| HSV1-UL19-3036 | + | CUCGCCGGGGGCGAUCU | 17 | 3010 |
| HSV1-UL19-1693 | + | CUGGGGCACUCCUCGCC | 17 | 2079 |
| HSV1-UL19-1694 | + | GCUGGGGCACUCCUCGC | 17 | 2080 |
| HSV1-UL19-3039 | + | CGCUGGGGCACUCCUCG | 17 | 3011 |
| HSV1-UL19-1697 | + | GUCCGUCACGGGGUCGC | 17 | 2083 |
| HSV1-UL19-3041 | + | GGUCCGUCACGGGGUCG | 17 | 3012 |
| HSV1-UL19-1700 | + | GUGCGCGGGGUCCGUCA | 17 | 2086 |
| HSV1-UL19-3043 | + | GGUGCGCGGGGUCCGUC | 17 | 3013 |
| HSV1-UL19-1703 | + | CGGGUGCAGGGGGUGCG | 17 | 2089 |
| HSV1-UL19-3045 | + | CCGGGUGCAGGGGGUGC | 17 | 3014 |
| HSV1-UL19-1706 | + | CAGAUUGGCCGGGUGCA | 17 | 2092 |
| HSV1-UL19-1707 | + | CCAGAUUGGCCGGGUGC | 17 | 2093 |
| HSV1-UL19-3048 | + | ACCAGAUUGGCCGGGUG | 17 | 3015 |
| HSV1-UL19-3049 | + | GUUGGCCACCAGAUUGG | 17 | 3016 |
| HSV1-UL19-3050 | + | ACUACCACGCGCCCGUU | 17 | 3017 |
| HSV1-UL19-3051 | + | CCUGCAGCGUGAGCAUG | 17 | 3018 |
| HSV1-UL19-3052 | + | GGGCCAGCACCUGCAGC | 17 | 3019 |
| HSV1-UL19-3053 | + | GUGCGUUCGGCCAUGUU | 17 | 3020 |
| HSV1-UL19-3054 | + | CGUCGGGCGCCGCCGAG | 17 | 3021 |
| HSV1-UL19-3055 | + | GCCCGCGUCGGGCGCCG | 17 | 3022 |
| HSV1-UL19-3056 | + | CGGUGUUGGCGCCCGCG | 17 | 3023 |
| HSV1-UL19-3057 | + | AUAUGCGCAUGUUGGUG | 17 | 3024 |
| HSV1-UL19-3058 | + | CGGCGUGCAACGCCCCG | 17 | 3025 |
| HSV1-UL19-3059 | + | UGCUGGGGGGCCAUCAG | 17 | 3026 |
| HSV1-UL19-1730 | + | GUAUGGUCCAGAUGCUG | 17 | 2116 |
| HSV1-UL19-1731 | + | GGUAUGGUCCAGAUGCU | 17 | 2117 |
| HSV1-UL19-1732 | + | UGGUAUGGUCCAGAUGC | 17 | 2118 |
| HSV1-UL19-3063 | + | AUGGUAUGGUCCAGAUG | 17 | 3027 |
| HSV1-UL19-3064 | + | UAAAAAUAGUCGCCAUU | 17 | 3028 |
| HSV1-UL19-1738 | + | CAGCGCGUGGACGGGGA | 17 | 2124 |
| HSV1-UL19-1739 | + | ACAGCGCGUGGACGGGG | 17 | 2125 |
| HSV1-UL19-3067 | + | AACAGCGCGUGGACGGG | 17 | 3029 |
| HSV1-UL19-1740 | + | CGAACAGCGCGUGGACG | 17 | 2126 |
| HSV1-UL19-1741 | + | GCGAACAGCGCGUGGAC | 17 | 2127 |
| HSV1-UL19-1742 | + | GGCGAACAGCGCGUGGA | 17 | 2128 |
| HSV1-UL19-3071 | + | CGGCGAACAGCGCGUGG | 17 | 3030 |
| HSV1-UL19-3072 | + | GCCCCGGCGAACAGCGC | 17 | 3031 |
| HSV1-UL19-3073 | + | CCACGUGGUCGGCCCCG | 17 | 3032 |
| HSV1-UL19-3074 | + | GCAGGGCCGGGGGGAAA | 17 | 3033 |
| HSV1-UL19-1749 | + | GGUCGCGCAGGGCCGGG | 17 | 2135 |
| HSV1-UL19-1750 | + | AGGUCGCGCAGGGCCGG | 17 | 2136 |
| HSV1-UL19-1751 | + | CAGGUCGCGCAGGGCCG | 17 | 2137 |
| HSV1-UL19-1752 | + | ACAGGUCGCGCAGGGCC | 17 | 2138 |
| HSV1-UL19-1753 | + | GACAGGUCGCGCAGGGC | 17 | 2139 |
| HSV1-UL19-3080 | + | CGACAGGUCGCGCAGGG | 17 | 3034 |
| HSV1-UL19-3081 | + | UGCCGCGACAGGUCGCG | 17 | 3035 |
| HSV1-UL19-1758 | + | AGAGCCGGGGGGACCAG | 17 | 2144 |
| HSV1-UL19-1759 | + | CAGAGCCGGGGGGACCA | 17 | 2145 |
| HSV1-UL19-1760 | + | CCAGAGCCGGGGGGACC | 17 | 2146 |
| HSV1-UL19-3085 | + | CCCAGAGCCGGGGGGAC | 17 | 3036 |
| HSV1-UL19-1762 | + | UUGGCCCCCAGAGCCGG | 17 | 2148 |
| HSV1-UL19-1763 | + | GUUGGCCCCCAGAGCCG | 17 | 2149 |
| HSV1-UL19-1764 | + | AGUUGGCCCCCAGAGCC | 17 | 2150 |
| HSV1-UL19-1765 | + | UAGUUGGCCCCCAGAGC | 17 | 2151 |
| HSV1-UL19-3090 | + | GUAGUUGGCCCCCAGAG | 17 | 3037 |
| HSV1-UL19-3091 | + | GAAAAGUAGUUGGCCCC | 17 | 3038 |
| HSV1-UL19-3092 | + | GACGGGCUGUCGGAUCG | 17 | 3039 |
| HSV1-UL19-3093 | + | UGCUGCACGACGGGCUG | 17 | 3040 |
| HSV1-UL19-3094 | + | CGCGGACGUGCUGCACG | 17 | 3041 |
| HSV1-UL19-3095 | + | UCCCCGGCCGCGCUCUC | 17 | 3042 |
| HSV1-UL19-3096 | + | UCUUGAAGUACCCCGCC | 17 | 3043 |
| HSV1-UL19-3097 | + | AGGCCACGGGACUGAUC | 17 | 3044 |
| HSV1-UL19-1774 | + | CUGAUGAUGCAAGGCCA | 17 | 2160 |
| HSV1-UL19-3099 | + | GCUGAUGAUGCAAGGCC | 17 | 3045 |
| HSV1-UL19-3100 | + | CGGGAUGGAGGCCCGUC | 17 | 3046 |
| HSV1-UL19-1777 | + | UGAACCCAAACCCGGGA | 17 | 2163 |
| HSV1-UL19-3102 | + | GUGAACCCAAACCCGGG | 17 | 3047 |
| HSV1-UL19-1779 | + | GACGGUGAACCCAAACC | 17 | 2165 |
| HSV1-UL19-3104 | + | CGACGGUGAACCCAAAC | 17 | 3048 |
| HSV1-UL19-3105 | + | GGUCCUGUCGGACGACG | 17 | 3049 |
| HSV1-UL19-3106 | + | GUCACAAAGCGGUCCUG | 17 | 3050 |
| HSV1-UL19-3107 | + | CCUCCGACGCGCGCUCC | 17 | 3051 |
| HSV1-UL19-3108 | + | CGCCUCCGACGCGCGCU | 17 | 3052 |
| HSV1-UL19-1783 | + | CCACCUGGAGCUGGCCC | 17 | 2169 |
| HSV1-UL19-3110 | + | GCCACCUGGAGCUGGCC | 17 | 3053 |
| HSV1-UL19-1785 | + | UUUCGUGCCGGGCCACC | 17 | 2171 |
| HSV1-UL19-3112 | + | GUUUCGUGCCGGGCCAC | 17 | 3054 |
| HSV1-UL19-3113 | + | CCCCCGCCAGUUUCGUG | 17 | 3055 |
| HSV1-UL19-3114 | + | CGCGCGGCUGGGUGAGC | 17 | 3056 |
| HSV1-UL19-3115 | + | CGUUCGCGCGCGGCUGG | 17 | 3057 |
| HSV1-UL19-3116 | + | UCCACGUUCGCGCGCGG | 17 | 3058 |
| HSV1-UL19-3117 | + | AUGUCGGUGACGGGGUU | 17 | 3059 |
| HSV1-UL19-1799 | + | GUUGCCCAUGUCGGUGA | 17 | 2185 |
| HSV1-UL19-3119 | + | GGUUGCCCAUGUCGGUG | 17 | 3060 |
| HSV1-UL19-1802 | + | CCCAGGUAAAAGUUUUG | 17 | 2188 |
| HSV1-UL19-1803 | + | GCCCAGGUAAAAGUUUU | 17 | 2189 |
| HSV1-UL19-1804 | + | GGCCCAGGUAAAAGUUU | 17 | 2190 |
| HSV1-UL19-3123 | + | CGGCCCAGGUAAAAGUU | 17 | 3061 |
| HSV1-UL19-1807 | + | GCGUUGUCCAGGAGAGG | 17 | 2193 |
| HSV1-UL19-1808 | + | CGCGUUGUCCAGGAGAG | 17 | 2194 |
| HSV1-UL19-1809 | + | CCGCGUUGUCCAGGAGA | 17 | 2195 |
| HSV1-UL19-1810 | + | GCCGCGUUGUCCAGGAG | 17 | 2196 |
| HSV1-UL19-3128 | + | UGCCGCGUUGUCCAGGA | 17 | 3062 |
| HSV1-UL19-3129 | + | GCUGCCGCGUUGUCCAG | 17 | 3063 |
| HSV1-UL19-1811 | + | CGGCUGCCGCGUUGUCC | 17 | 2197 |
| HSV1-UL19-3131 | + | ACGGCUGCCGCGUUGUC | 17 | 3064 |
| HSV1-UL19-3132 | + | CCGAACACGGGGACGGG | 17 | 3065 |
| HSV1-UL19-3133 | + | CGCACCCGAACACGGGG | 17 | 3066 |
| HSV1-UL19-1822 | + | UGGGCGCACCCGAACAC | 17 | 2208 |
| HSV1-UL19-1823 | + | CUGGGCGCACCCGAACA | 17 | 2209 |
| HSV1-UL19-3136 | + | CCUGGGCGCACCCGAAC | 17 | 3067 |
| HSV1-UL19-3137 | + | GCGGCACCUGGGCGCAC | 17 | 3068 |
| HSV1-UL19-3138 | + | CCUGCGCGCCGCGGCAC | 17 | 3069 |
| HSV1-UL19-1833 | + | UUGACGUCGGUCGACAC | 17 | 2219 |
| HSV1-UL19-1834 | + | GUUGACGUCGGUCGACA | 17 | 2220 |
| HSV1-UL19-3141 | + | AGUUGACGUCGGUCGAC | 17 | 3070 |
| HSV1-UL19-1836 | + | GGGGGUUGCAGGGCCGG | 17 | 2222 |
| HSV1-UL19-3143 | + | CGGGGGUUGCAGGGCCG | 17 | 3071 |
| HSV1-UL19-3144 | + | CGCGUCCCCGGGGGUUG | 17 | 3072 |
| HSV1-UL19-1842 | + | CCCGGCGGCGCGUCCCC | 17 | 2228 |
| HSV1-UL19-1843 | + | CCCCGGCGGCGCGUCCC | 17 | 2229 |
| HSV1-UL19-3147 | + | CCCCCGGCGGCGCGUCC | 17 | 3073 |
| HSV1-UL19-3148 | + | UGGCCGUGGUCGUACAU | 17 | 3074 |
| HSV1-UL19-3149 | + | UCUGGCCGUGGUCGUAC | 17 | 3075 |
| HSV1-UL19-3150 | + | GGCCGCGAAGGCCCGGG | 17 | 3076 |
| HSV1-UL19-1854 | + | CCGUGGCCGCGAAGGCC | 17 | 2240 |
| HSV1-UL19-3152 | + | GCCGUGGCCGCGAAGGC | 17 | 3077 |
| HSV1-UL19-3153 | + | ACGGGUUGGCCGUGGCC | 17 | 3078 |
| HSV1-UL19-3154 | + | AAAUCGCUGCGACGCCC | 17 | 3079 |
| HSV1-UL19-3155 | + | AUAGAGCAGGUCCCCGU | 17 | 3080 |
| HSV1-UL19-3156 | + | GGUGGUAGGCCCCGUUA | 17 | 3081 |
| HSV1-UL19-3157 | + | GCACCGGCGAGGCCCCG | 17 | 3082 |
| HSV1-UL19-3158 | + | GCAGGGGCUGAGCACCG | 17 | 3083 |
| HSV1-UL19-3159 | + | AGAACUUAAAGCAGGGG | 17 | 3084 |
| HSV1-UL19-1868 | + | CGUAAAGAACUUAAAGC | 17 | 2254 |
| HSV1-UL19-3161 | + | ACGUAAAGAACUUAAAG | 17 | 3085 |
| HSV1-UL19-3162 | + | CGAUGUCGGCCGACGUA | 17 | 3086 |
| HSV1-UL19-3163 | + | GGUGGCCGUGGACACCG | 17 | 3087 |
| HSV1-UL19-3164 | + | CGCUGGCGGCGGUGGCC | 17 | 3088 |
| HSV1-UL19-1881 | + | GAGUUCGCGGCACCCCG | 17 | 2267 |
| HSV1-UL19-1882 | + | CGAGUUCGCGGCACCCC | 17 | 2268 |
| HSV1-UL19-1883 | + | ACGAGUUCGCGGCACCC | 17 | 2269 |
| HSV1-UL19-3168 | + | CACGAGUUCGCGGCACC | 17 | 3089 |
| HSV1-UL19-3169 | + | GGCCACACGGGUCCUCC | 17 | 3090 |
| HSV1-UL19-3170 | + | CUCCUGAAACAGGCCAC | 17 | 3091 |
| HSV1-UL19-3171 | + | GUGAGCGGGUAGGCCUC | 17 | 3092 |
| HSV1-UL19-3172 | + | GUCGCUGGCGCAGGUGA | 17 | 3093 |
| HSV1-UL19-3173 | + | CGGGGUCGCUGGCGCAG | 17 | 3094 |
| HSV1-UL19-1895 | + | GGCACUGCGGAGGAGGG | 17 | 2281 |
| HSV1-UL19-3175 | + | GGGCACUGCGGAGGAGG | 17 | 3095 |
| HSV1-UL19-3176 | + | UUGCGGGCACUGCGGAG | 17 | 3096 |
| HSV1-UL19-1898 | + | CGUUGCGGGCACUGCGG | 17 | 2284 |
| HSV1-UL19-3178 | + | CCGUUGCGGGCACUGCG | 17 | 3097 |
| HSV1-UL19-1899 | + | CCCCGUUGCGGGCACUG | 17 | 2285 |
| HSV1-UL19-3180 | + | UCCCCGUUGCGGGCACU | 17 | 3098 |
| HSV1-UL19-3181 | + | GCGUGGGCUUCCCCGUU | 17 | 3099 |
| HSV1-UL19-3182 | + | AAGUGGGUCUCCCGCGC | 17 | 3100 |
| HSV1-UL19-3183 | + | ACGAGAUACUGCGCGAA | 17 | 3101 |
| HSV1-UL19-3184 | + | CAUAGACGAGAUACUGC | 17 | 3102 |
| HSV1-UL19-3185 | + | GCGGGGAUGCGUCAUAG | 17 | 3103 |
| HSV1-UL19-1907 | + | GAGCCAGUCCCUUGAGC | 17 | 2293 |
| HSV1-UL19-1908 | + | AGAGCCAGUCCCUUGAG | 17 | 2294 |
| HSV1-UL19-3188 | + | CAGAGCCAGUCCCUUGA | 17 | 3104 |

Table 1F provides exemplary targeting domains for knocking out the UL19 gene selected according to the first tier parameters. The targeting domains are selected based on location within first 500 bp of the coding sequence of the UL19 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 1F

| | | | Target | |
|---|---|---|---|---|
| 1st Tier | DNA | | Site | Seq |
| gRNA Name | Strand | Targeting Domain | Length | ID |
| HSV1-UL19-3189 | − | GUGGCGUCGCAUCGACG | 20 | 3105 |
| | | CCU | | |
| HSV1-UL19-3190 | − | ACAGUUCGAGGUCCACC | 20 | 3106 |
| | | AGC | | |
| HSV1-UL19-3191 | − | ACCCACACACAAUUACA | 20 | 3107 |
| | | UGA | | |
| HSV1-UL19-3192 | − | GCGUCGCAUCGACGCCU | 17 | 3108 |
| HSV1-UL19-3193 | − | GUUCGAGGUCCACCAGC | 17 | 3109 |
| HSV1-UL19-3194 | − | CACACACAAUUACAUGA | 17 | 3110 |

Table 1G provides exemplary targeting domains for knocking out the UL19 gene selected according to the second tier parameters. The targeting domains are selected based on location within the coding sequence, but downstream of the first 500 bp of the UL19 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 1G

| | | | Target | |
|---|---|---|---|---|
| 2nd Tier | DNA | | Site | Seq |
| gRNA Name | Strand | Targeting Domain | Length | ID |
| HSV1-UL19-3195 | − | CGGCCAACCCGUGGGCG | 20 | 3111 |
| | | UCG | | |
| HSV1-UL19-3196 | − | GGCGGACUGGACCGUGC | 20 | 3112 |
| | | ACC | | |
| HSV1-UL19-3197 | − | CCAACCCGUGGGCGUCG | 17 | 3113 |
| HSV1-UL19-3198 | − | GGACUGGACCGUGCACC | 17 | 3114 |
| HSV1-UL19-3199 | + | GCGCGGCAAACCGUUCC | 20 | 3115 |
| | | AUG | | |
| HSV1-UL19-3200 | + | GGGCCGCCACGUACGCC | 20 | 3116 |
| | | CCG | | |
| HSV1-UL19-1181 | + | GCAGCGCCGGGUCUCGC | 20 | 1567 |
| | | AUU | | |
| HSV1-UL19-3202 | + | UGGCGUUGACCGUGUUG | 20 | 3117 |
| | | GCC | | |
| HSV1-UL19-3203 | + | CAGAUGCUGGGGGGCCA | 20 | 3118 |
| | | UCA | | |
| HSV1-UL19-3204 | + | CGGCAAACCGUUCCAUG | 17 | 3119 |
| HSV1-UL19-3205 | + | CCGCCACGUACGCCCCG | 17 | 3120 |
| HSV1-UL19-1648 | + | GCGCCGGGUCUCGCAUU | 17 | 2034 |
| HSV1-UL19-3207 | + | CGUUGACCGUGUUGGCC | 17 | 3121 |
| HSV1-UL19-3208 | + | AUGCUGGGGGGCCAUCA | 17 | 3122 |

Table 2A provides exemplary targeting domains for knocking out the UL30 gene selected according to first tier parameters. The targeting domains are selected based on location within the first 500 bp of the coding sequence of the UL30 gene and orthogonality against the human genome. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2A

| | | | Target | |
|---|---|---|---|---|
| 1st Tier | DNA | | Site | Seq |
| gRNA Name | Strand | Targeting Domain | Length | ID |
| HSV1-UL30-4 | − | GUCCCCCGGAGGAAAGUCGG | 20 | 3123 |
| HSV1-UL30-5 | − | CCGGAGGAAAGUCGGCGGCC | 20 | 3124 |
| HSV1-UL30-11 | − | UUUGCGCCCGCCGGCCCUCG | 20 | 3125 |
| HSV1-UL30-18 | − | GGGACGCAACAGAAGCCGAC | 20 | 3126 |
| HSV1-UL30-19 | − | GGACGCAACAGAAGCCGACC | 20 | 3127 |
| HSV1-UL30-20 | − | AAUUUCGAUUCAUCGCCCCG | 20 | 3128 |
| HSV1-UL30-21 | − | AUUUCGAUUCAUCGCCCCGC | 20 | 3129 |
| HSV1-UL30-22 | − | AUUCAUCGCCCCGCGGGUGC | 20 | 3130 |
| HSV1-UL30-25 | − | GCCCCCCCGGAGAAGCGCGC | 20 | 3131 |
| HSV1-UL30-27 | − | CCCCCCGGAGAAGCGCGCCG | 20 | 3132 |
| HSV1-UL30-28 | − | AAGCGCGCCGGGGUGCACGA | 20 | 3133 |
| HSV1-UL30-29 | − | UCACCUCAAGCGCGCCCCCA | 20 | 3134 |
| HSV1-UL30-30 | − | CGCGCCCCCAAGGUGUACUG | 20 | 3135 |
| HSV1-UL30-31 | − | GCGCCCCCAAGGUGUACUGC | 20 | 3136 |
| HSV1-UL30-32 | − | CGCCCCCAAGGUGUACUGCG | 20 | 3137 |
| HSV1-UL30-33 | − | GCCCCCAAGGUGUACUGCGG | 20 | 3138 |
| HSV1-UL30-36 | − | GAGCGCGACGUCCUCCGCGU | 20 | 3139 |
| HSV1-UL30-37 | − | AGCGCGACGUCCUCCGCGUC | 20 | 3140 |
| HSV1-UL30-38 | − | CGACGUCCUCCGCGUCGGGU | 20 | 3141 |
| HSV1-UL30-39 | − | GACGUCCUCCGCGUCGGGUC | 20 | 3142 |
| HSV1-UL30-40 | − | GUCCUCCGCGUCGGGUCGGG | 20 | 3143 |
| HSV1-UL30-50 | − | GGCGUGGACCACGCCCCGGC | 20 | 3144 |
| HSV1-UL30-51 | − | GCGUGGACCACGCCCCGGCG | 20 | 3145 |
| HSV1-UL30-52 | − | CUUUCACGUGUACGACAUCC | 20 | 3146 |
| HSV1-UL30-53 | − | GUACGACAUCCUGGAGAACG | 20 | 3147 |
| HSV1-UL30-54 | − | GAGAACGUGGAGCACGCGUA | 20 | 3148 |
| HSV1-UL30-56 | + | ACGCGUGCUCCACGUUCUCC | 20 | 3149 |
| HSV1-UL30-57 | + | GAUGUCGUACACGUGAAAGA | 20 | 3150 |
| HSV1-UL30-61 | + | CGUGAAAGACGGUGACGGUG | 20 | 3151 |
| HSV1-UL30-62 | + | ACGGUGGGGUUGAACCCCGC | 20 | 3152 |
| HSV1-UL30-64 | + | GGUGGGGUUGAACCCCGCCG | 20 | 3153 |
| HSV1-UL30-65 | + | GGUUGAACCCCGCCGGGGCG | 20 | 3154 |
| HSV1-UL30-68 | + | AGAAGCCGCCCGACCCGACG | 20 | 3155 |
| HSV1-UL30-69 | + | AGCCGCCCGACCCGACGCGG | 20 | 3156 |
| HSV1-UL30-70 | + | GUCCCCCCCGCAGUACACCU | 20 | 3157 |
| HSV1-UL30-74 | + | ACACCUUGGGGGCGCGCUUG | 20 | 3158 |
| HSV1-UL30-75 | + | GAGGUGACCGUCGUGCACCC | 20 | 3159 |
| HSV1-UL30-80 | + | CCCCGGCGCGCUUCUCCGGG | 20 | 3160 |
| HSV1-UL30-82 | + | GCAUCCUCGUCCAGCACCCG | 20 | 3161 |
| HSV1-UL30-84 | + | AUCCUCGUCCAGCACCCGCG | 20 | 3162 |
| HSV1-UL30-87 | + | GCUAUAGUACGUAUGGCGCU | 20 | 3163 |
| HSV1-UL30-88 | + | UAGUACGUAUGGCGCUGGGU | 20 | 3164 |
| HSV1-UL30-89 | + | CGUAUGGCGCUGGGUUGGCC | 20 | 3165 |
| HSV1-UL30-91 | + | GGCUUCUGUUGCGUCCCGAC | 20 | 3166 |
| HSV1-UL30-92 | + | GCUUCUGUUGCGUCCCGACU | 20 | 3167 |
| HSV1-UL30-93 | + | CUUCUGUUGCGUCCCGACUG | 20 | 3168 |
| HSV1-UL30-94 | + | GUUGCGUCCCGACUGGGGCG | 20 | 3169 |
| HSV1-UL30-95 | + | CGUCCCGACUGGGGCGAGGU | 20 | 3170 |
| HSV1-UL30-101 | + | GUUUUGCCUCAAACAAGGCG | 20 | 3171 |
| HSV1-UL30-102 | + | UUUUGCCUCAAACAAGGCGG | 20 | 3172 |
| HSV1-UL30-103 | + | UCAAACAAGGCGGGGGUCCC | 20 | 3173 |
| HSV1-UL30-110 | + | GCCGGCGGGCGCAAAAAACC | 20 | 3174 |
| HSV1-UL30-111 | + | AAAAAACCCGGACGCCGCCC | 20 | 3175 |
| HSV1-UL30-114 | + | UGGCCGCCGACUUUCCUCCG | 20 | 3176 |
| HSV1-UL30-115 | + | GGCCGCCGACUUUCCUCCGG | 20 | 3177 |
| HSV1-UL30-121 | − | GUCCCCCGGAGGAAAGU | 17 | 3178 |
| HSV1-UL30-125 | − | AAAGUCGGCGGCCAGGG | 17 | 3179 |
| HSV1-UL30-128 | − | GGGUUUUUUGCGCCCGC | 17 | 3180 |
| HSV1-UL30-136 | − | ACGCAACAGAAGCCGAC | 17 | 3181 |
| HSV1-UL30-137 | − | CGCAACAGAAGCCGACC | 17 | 3182 |
| HSV1-UL30-138 | − | UUCGAUUCAUCGCCCCG | 17 | 3183 |
| HSV1-UL30-139 | − | UCGAUUCAUCGCCCCGC | 17 | 3184 |
| HSV1-UL30-140 | − | CAUCGCCCCGCGGGUGC | 17 | 3185 |
| HSV1-UL30-144 | − | CCCCGGAGAAGCGCGCC | 17 | 3186 |
| HSV1-UL30-146 | − | CGCGCCGGGGUGCACGA | 17 | 3187 |
| HSV1-UL30-147 | − | CCUCAAGCGCGCCCCCA | 17 | 3188 |
| HSV1-UL30-148 | − | GCCCCCAAGGUGUACUG | 17 | 3189 |
| HSV1-UL30-150 | − | CCCCAAGGUGUACUGCG | 17 | 3190 |
| HSV1-UL30-155 | − | GCGACGUCCUCCGCGUC | 17 | 3191 |
| HSV1-UL30-156 | − | CGUCCUCCGCGUCGGGU | 17 | 3192 |
| HSV1-UL30-157 | − | GUCCUCCGCGUCGGGUC | 17 | 3193 |
| HSV1-UL30-158 | − | CUCCGCGUCGGGUCGGG | 17 | 3194 |
| HSV1-UL30-163 | − | CGGCGCUCGCGCCUGUG | 17 | 3195 |
| HSV1-UL30-164 | − | CGCUCGCGCCUGUGGGG | 17 | 3196 |
| HSV1-UL30-167 | − | CGUGGACCACGCCCCGG | 17 | 3197 |
| HSV1-UL30-168 | − | GUGGACCACGCCCCGGC | 17 | 3198 |
| HSV1-UL30-169 | − | UGGACCACGCCCCGGCG | 17 | 3199 |
| HSV1-UL30-170 | − | UCACGUGUACGACAUCC | 17 | 3200 |
| HSV1-UL30-171 | − | CGACAUCCUGGAGAACG | 17 | 3201 |
| HSV1-UL30-172 | − | AACGUGGAGCACGCGUA | 17 | 3202 |
| HSV1-UL30-175 | + | GUCGUACACGUGAAAGA | 17 | 3203 |
| HSV1-UL30-178 | + | UGAAAGACGGUGACGGU | 17 | 3204 |
| HSV1-UL30-182 | + | GGGGUUGAACCCCGCCG | 17 | 3205 |
| HSV1-UL30-185 | + | CACAGGCGCGAGCGCCG | 17 | 3206 |
| HSV1-UL30-186 | + | AGCCGCCCGACCCGACG | 17 | 3207 |
| HSV1-UL30-187 | + | CGCCCGACCCGACGCGG | 17 | 3208 |
| HSV1-UL30-188 | + | CCCCCCGCAGUACACCU | 17 | 3209 |
| HSV1-UL30-189 | + | CCCCCGCAGUACACCUU | 17 | 3210 |
| HSV1-UL30-190 | + | CCCCGCAGUACACCUUG | 17 | 3211 |
| HSV1-UL30-193 | + | GUGACCGUCGUGCACCC | 17 | 3212 |
| HSV1-UL30-196 | + | CCCGGCGCGCUUCUCCG | 17 | 3213 |
| HSV1-UL30-197 | + | CCGGCGCGCUUCUCCGG | 17 | 3214 |
| HSV1-UL30-198 | + | CGGCGCGCUUCUCCGGG | 17 | 3215 |
| HSV1-UL30-199 | + | GGCGCGCUUCUCCGGGG | 17 | 3216 |
| HSV1-UL30-202 | + | CUCGUCCAGCACCCGCG | 17 | 3217 |
| HSV1-UL30-203 | + | AUUCGCUAUAGUACGUA | 17 | 3218 |
| HSV1-UL30-204 | + | UAUAGUACGUAUGGCGC | 17 | 3219 |
| HSV1-UL30-205 | + | AUAGUACGUAUGGCGCU | 17 | 3220 |
| HSV1-UL30-206 | + | UACGUAUGGCGCUGGGU | 17 | 3221 |
| HSV1-UL30-209 | + | UUCUGUUGCGUCCCGAC | 17 | 3222 |
| HSV1-UL30-210 | + | UCUGUUGCGUCCCGACU | 17 | 3223 |
| HSV1-UL30-211 | + | CUGUUGCGUCCCGACUG | 17 | 3224 |
| HSV1-UL30-212 | + | GCGUCCCGACUGGGGCG | 17 | 3225 |
| HSV1-UL30-215 | + | CGACUGGGGCGAGGUAG | 17 | 3226 |
| HSV1-UL30-228 | + | GGCGGGCGCAAAAAACC | 17 | 3227 |
| HSV1-UL30-230 | + | GGCCGCCGACUUUCCUC | 17 | 3228 |
| HSV1-UL30-231 | + | GCCGCCGACUUUCCUCC | 17 | 3229 |
| HSV1-UL30-232 | + | CCGCCGACUUUCCUCCG | 17 | 3230 |
| HSV1-UL30-233 | + | CGCCGACUUUCCUCCGG | 17 | 3231 |

Table 2B provides exemplary targeting domains for knocking out the UL30 gene selected according to the second tier parameters. The targeting domains are selected based on location within the first 500 bp of the coding sequence of the UL30 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2B

| | | | Target | |
|---|---|---|---|---|
| 2nd Tier | DNA | | Site | |
| gRNA Name | Strand | Targeting Domain | Length | Seq ID |
| HSV1-UL30-1 | − | GGCGGCGGCCCGCUGUCCCC | 20 | 3232 |
| HSV1-UL30-2 | − | GGCGGCCCGCUGUCCCCCGG | 20 | 3233 |
| HSV1-UL30-3 | − | GCUGUCCCCCGGAGGAAAGU | 20 | 3234 |
| HSV1-UL30-6 | − | CGGAGGAAAGUCGGCGGCCA | 20 | 3235 |
| HSV1-UL30-7 | − | AGGAAAGUCGGCGGCCAGGG | 20 | 3236 |
| HSV1-UL30-8 | − | UCGGCGGCCAGGGCGGCGUC | 20 | 3237 |
| HSV1-UL30-9 | − | CGGCGGCCAGGGCGGCGUCC | 20 | 3238 |
| HSV1-UL30-10 | − | UCCGGGUUUUUUGCGCCCGC | 20 | 3239 |
| HSV1-UL30-12 | − | CCGGCCCUCGCGGAGCCAGC | 20 | 3240 |
| HSV1-UL30-13 | − | CGGCCCUCGCGGAGCCAGCC | 20 | 3241 |
| HSV1-UL30-14 | − | GGCCCUCGCGGAGCCAGCCG | 20 | 3242 |
| HSV1-UL30-15 | − | GGGGACCCCCGCCUUGUUUG | 20 | 3243 |
| HSV1-UL30-16 | − | AACCCCUACCUCGCCCCAGU | 20 | 3244 |
| HSV1-UL30-17 | − | ACCCCUACCUCGCCCCAGUC | 20 | 3245 |
| HSV1-UL30-23 | − | CGCCCCGCGGGUGCUGGACG | 20 | 3246 |
| HSV1-UL30-24 | − | GCUGGACGAGGAUGCCCCCC | 20 | 3247 |
| HSV1-UL30-26 | − | CCCCCCCGGAGAAGCGCGCC | 20 | 3248 |
| HSV1-UL30-34 | − | CCCCCAAGGUGUACUGCGGG | 20 | 3249 |
| HSV1-UL30-35 | − | CCCCAAGGUGUACUGCGGGG | 20 | 3250 |
| HSV1-UL30-41 | − | GCGUCGGGUCGGGCGGCUUC | 20 | 3251 |
| HSV1-UL30-42 | − | GGUCGGGCGGCUUCUGGCCG | 20 | 3252 |
| HSV1-UL30-43 | − | GGCCGCGGCGCUCGCGCCUG | 20 | 3253 |
| HSV1-UL30-44 | − | GCCGCGGCGCUCGCGCCUGU | 20 | 3254 |
| HSV1-UL30-45 | − | CCGCGGCGCUCGCGCCUGUG | 20 | 3255 |
| HSV1-UL30-46 | − | CGGCGCUCGCGCCUGUGGGG | 20 | 3256 |
| HSV1-UL30-47 | − | CUCGCGCCUGUGGGGCGGCG | 20 | 3257 |
| HSV1-UL30-48 | − | GGGCGGCGUGGACCACGCCC | 20 | 3258 |
| HSV1-UL30-49 | − | CGGCGUGGACCACGCCCCGG | 20 | 3259 |
| HSV1-UL30-55 | − | GCACGCGUACGGCAUGCGCG | 20 | 3260 |
| HSV1-UL30-58 | + | GUACACGUGAAAGACGGUGA | 20 | 3261 |
| HSV1-UL30-59 | + | CACGUGAAAGACGGUGACGG | 20 | 3262 |
| HSV1-UL30-60 | + | ACGUGAAAGACGGUGACGGU | 20 | 3263 |
| HSV1-UL30-63 | + | CGGUGGGGUUGAACCCCGCC | 20 | 3264 |
| HSV1-UL30-66 | + | CGUGGUCCACGCCGCCCCAC | 20 | 3265 |
| HSV1-UL30-67 | + | CCCCACAGGCGCGAGCGCCG | 20 | 3266 |
| HSV1-UL30-71 | + | UCCCCCCCGCAGUACACCUU | 20 | 3267 |
| HSV1-UL30-72 | + | CCCCCCCGCAGUACACCUUG | 20 | 3268 |
| HSV1-UL30-73 | + | CCCCCCGCAGUACACCUUGG | 20 | 3269 |
| HSV1-UL30-76 | + | UGCACCCCGGCGCGCUUCUC | 20 | 3270 |
| HSV1-UL30-77 | + | GCACCCCGGCGCGCUUCUCC | 20 | 3271 |
| HSV1-UL30-78 | + | CACCCCGGCGCGCUUCUCCG | 20 | 3272 |
| HSV1-UL30-79 | + | ACCCCGGCGCGCUUCUCCGG | 20 | 3273 |
| HSV1-UL30-81 | + | CCCGGCGCGCUUCUCCGGGG | 20 | 3274 |
| HSV1-UL30-83 | + | CAUCCUCGUCCAGCACCCGC | 20 | 3275 |
| HSV1-UL30-85 | + | CGCAUUCGCUAUAGUACGUA | 20 | 3276 |
| HSV1-UL30-86 | + | CGCUAUAGUACGUAUGGCGC | 20 | 3277 |
| HSV1-UL30-90 | + | UGGCGCUGGGUUGGCCCGGU | 20 | 3278 |
| HSV1-UL30-96 | + | GUCCCGACUGGGGCGAGGUA | 20 | 3279 |
| HSV1-UL30-97 | + | UCCCGACUGGGGCGAGGUAG | 20 | 3280 |
| HSV1-UL30-98 | + | UAAAAGUUUUGCCUCAAACA | 20 | 3281 |
| HSV1-UL30-99 | + | AAGUUUUGCCUCAAACAAGG | 20 | 3282 |
| HSV1-UL30-100 | + | AGUUUUGCCUCAAACAAGGC | 20 | 3283 |
| HSV1-UL30-104 | + | ACAAGGCGGGGGUCCCCGGC | 20 | 3284 |
| HSV1-UL30-105 | + | GGUCCCCGGCUGGCUCCGCG | 20 | 3285 |
| HSV1-UL30-106 | + | GUCCCCGGCUGGCUCCGCGA | 20 | 3286 |
| HSV1-UL30-107 | + | CCGGCUGGCUCCGCGAGGGC | 20 | 3287 |
| HSV1-UL30-108 | + | GCUGGCUCCGCGAGGGCCGG | 20 | 3288 |
| HSV1-UL30-109 | + | CUGGCUCCGCGAGGGCCGGC | 20 | 3289 |
| HSV1-UL30-112 | + | CCUGGCCGCCGACUUUCCUC | 20 | 3290 |
| HSV1-UL30-113 | + | CUGGCCGCCGACUUUCCUCC | 20 | 3291 |
| HSV1-UL30-116 | + | GACUUUCCUCCGGGGGACAG | 20 | 3292 |
| HSV1-UL30-117 | + | ACUUUCCUCCGGGGGACAGC | 20 | 3293 |
| HSV1-UL30-118 | + | GGACAGCGGGCCGCCGCCAC | 20 | 3294 |
| HSV1-UL30-119 | − | GGCGGCCCGCUGUCCCC | 17 | 3295 |
| HSV1-UL30-120 | − | GGCCCGCUGUCCCCCGG | 17 | 3296 |
| HSV1-UL30-122 | − | CCCCGGAGGAAAGUCGG | 17 | 3297 |
| HSV1-UL30-123 | − | GAGGAAAGUCGGCGGCC | 17 | 3298 |
| HSV1-UL30-124 | − | AGGAAAGUCGGCGGCCA | 17 | 3299 |
| HSV1-UL30-126 | − | GCGGCCAGGGCGGCGUC | 17 | 3300 |
| HSV1-UL30-127 | − | CGGCCAGGGCGGCGUCC | 17 | 3301 |
| HSV1-UL30-129 | − | GCGCCCGCCGGCCCUCG | 17 | 3302 |
| HSV1-UL30-130 | − | GCCCUCGCGGAGCCAGC | 17 | 3303 |
| HSV1-UL30-131 | − | CCCUCGCGGAGCCAGCC | 17 | 3304 |
| HSV1-UL30-132 | − | CCUCGCGGAGCCAGCCG | 17 | 3305 |
| HSV1-UL30-133 | − | GACCCCCGCCUUGUUUG | 17 | 3306 |
| HSV1-UL30-134 | − | CCCUACCUCGCCCCAGU | 17 | 3307 |
| HSV1-UL30-135 | − | CCUACCUCGCCCCAGUC | 17 | 3308 |
| HSV1-UL30-141 | − | CCCGCGGGUGCUGGACG | 17 | 3309 |
| HSV1-UL30-142 | − | GGACGAGGAUGCCCCCC | 17 | 3310 |
| HSV1-UL30-143 | − | CCCCCGGAGAAGCGCGC | 17 | 3311 |
| HSV1-UL30-145 | − | CCCGGAGAAGCGCGCCG | 17 | 3312 |
| HSV1-UL30-149 | − | CCCCCAAGGUGUACUGC | 17 | 3313 |
| HSV1-UL30-151 | − | CCCAAGGUGUACUGCGG | 17 | 3314 |
| HSV1-UL30-152 | − | CCAAGGUGUACUGCGGG | 17 | 3315 |
| HSV1-UL30-153 | − | CAAGGUGUACUGCGGGG | 17 | 3316 |
| HSV1-UL30-154 | − | CGCGACGUCCUCCGCGU | 17 | 3317 |
| HSV1-UL30-159 | − | UCGGGUCGGGCGGCUUC | 17 | 3318 |
| HSV1-UL30-160 | − | CGGGCGGCUUCUGGCCG | 17 | 3319 |
| HSV1-UL30-161 | − | CGCGGCGCUCGCGCCUG | 17 | 3320 |
| HSV1-UL30-162 | − | GCGGCGCUCGCGCCUGU | 17 | 3321 |
| HSV1-UL30-165 | − | GCGCCUGUGGGGCGGCG | 17 | 3322 |
| HSV1-UL30-166 | − | CGGCGUGGACCACGCCC | 17 | 3323 |
| HSV1-UL30-173 | − | CGCGUACGGCAUGCGCG | 17 | 3324 |
| HSV1-UL30-174 | + | CGUGCUCCACGUUCUCC | 17 | 3325 |
| HSV1-UL30-176 | + | CACGUGAAAGACGGUGA | 17 | 3326 |
| HSV1-UL30-177 | + | GUGAAAGACGGUGACGG | 17 | 3327 |
| HSV1-UL30-179 | + | GAAAGACGGUGACGGUG | 17 | 3328 |
| HSV1-UL30-180 | + | GUGGGGUUGAACCCCGC | 17 | 3329 |
| HSV1-UL30-181 | + | UGGGGUUGAACCCCGCC | 17 | 3330 |
| HSV1-UL30-183 | + | UGAACCCCGCCGGGGCG | 17 | 3331 |
| HSV1-UL30-184 | + | GGUCCACGCCGCCCCAC | 17 | 3332 |
| HSV1-UL30-191 | + | CCCGCAGUACACCUUGG | 17 | 3333 |
| HSV1-UL30-192 | + | CCUUGGGGGCGCGCUUG | 17 | 3334 |
| HSV1-UL30-194 | + | ACCCCGGCGCGCUUCUC | 17 | 3335 |
| HSV1-UL30-195 | + | CCCCGGCGCGCUUCUCC | 17 | 3336 |
| HSV1-UL30-200 | + | UCCUCGUCCAGCACCCG | 17 | 3337 |
| HSV1-UL30-201 | + | CCUCGUCCAGCACCCGC | 17 | 3338 |
| HSV1-UL30-207 | + | AUGGCGCUGGGUUGGCC | 17 | 3339 |
| HSV1-UL30-208 | + | CGCUGGGUUGGCCCGGU | 17 | 3340 |
| HSV1-UL30-213 | + | CCCGACUGGGGCGAGGU | 17 | 3341 |
| HSV1-UL30-214 | + | CCGACUGGGGCGAGGUA | 17 | 3342 |
| HSV1-UL30-216 | + | AAGUUUUGCCUCAAACA | 17 | 3343 |
| HSV1-UL30-217 | + | UUUUGCCUCAAACAAGG | 17 | 3344 |
| HSV1-UL30-218 | + | UUUGCCUCAAACAAGGC | 17 | 3345 |
| HSV1-UL30-219 | + | UUGCCUCAAACAAGGCG | 17 | 3346 |
| HSV1-UL30-220 | + | UGCCUCAAACAAGGCGG | 17 | 3347 |
| HSV1-UL30-221 | + | AACAAGGCGGGGGUCCC | 17 | 3348 |
| HSV1-UL30-222 | + | AGGCGGGGGUCCCCGGC | 17 | 3349 |
| HSV1-UL30-223 | + | CCCCGGCUGGCUCCGCG | 17 | 3350 |
| HSV1-UL30-224 | + | CCCGGCUGGCUCCGCGA | 17 | 3351 |
| HSV1-UL30-225 | + | GCUGGCUCCGCGAGGGC | 17 | 3352 |
| HSV1-UL30-226 | + | GGCUCCGCGAGGGCCGG | 17 | 3353 |
| HSV1-UL30-227 | + | GCUCCGCGAGGGCCGGC | 17 | 3354 |
| HSV1-UL30-229 | + | AAACCCGGACGCCGCCC | 17 | 3355 |
| HSV1-UL30-234 | + | UUUCCUCCGGGGGACAG | 17 | 3356 |
| HSV1-UL30-235 | + | UUCCUCCGGGGGACAGC | 17 | 3357 |
| HSV1-UL30-236 | + | CAGCGGGCCGCCGCCAC | 17 | 3358 |

Table 2C provides exemplary targeting domains for knocking out the UL30 gene selected according to the third tier parameters. The targeting domains are selected based on location within the coding sequence, but downstream of the first 500 bp of the UL30 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2C

| | | | Target | |
|---|---|---|---|---|
| 3rd Tier | DNA | | Site | |
| gRNA Name | Strand | Targeting Domain | Length | Seq ID |
| HSV1-UL30-421 | − | TATGGACGCCATCACACCGA | 20 | 3359 |
| HSV1-UL30-422 | − | ATGGACGCCATCACACCGAC | 20 | 3360 |
| HSV1-UL30-423 | − | TGGACGCCATCACACCGACG | 20 | 3361 |
| HSV1-UL30-424 | − | GGGGACCGTCATCACGCTCC | 20 | 3362 |
| HSV1-UL30-425 | − | GGGACCGTCATCACGCTCCT | 20 | 3363 |
| HSV1-UL30-426 | − | CACGCTCCTGGGCCTGACTC | 20 | 3364 |
| HSV1-UL30-427 | − | CTCCTGGGCCTGACTCCGGA | 20 | 3365 |
| HSV1-UL30-428 | − | GCCTGACTCCGGAAGGCCAC | 20 | 3366 |
| HSV1-UL30-429 | − | CCTGACTCCGGAAGGCCACC | 20 | 3367 |
| HSV1-UL30-430 | − | GACTCCGGAAGGCCACCGGG | 20 | 3368 |
| HSV1-UL30-431 | − | CGGGTGGCCGTTCACGTTTA | 20 | 3369 |
| HSV1-UL30-432 | − | CCGTTCACGTTTACGGCACG | 20 | 3370 |
| HSV1-UL30-433 | − | GCAGTACTTTTACATGAACA | 20 | 3371 |
| HSV1-UL30-434 | − | GTACTTTTACATGAACAAGG | 20 | 3372 |
| HSV1-UL30-435 | − | CTTTTACATGAACAAGGAGG | 20 | 3373 |
| HSV1-UL30-436 | − | TGAACAAGGAGGAGGTCGAC | 20 | 3374 |
| HSV1-UL30-437 | − | ACGAGATCTCTGCGAGCGCA | 20 | 3375 |
| HSV1-UL30-438 | − | TCTCTGCGAGCGCATGGCCG | 20 | 3376 |
| HSV1-UL30-439 | − | CGCGGCCCTGCGCGAGTCCC | 20 | 3377 |
| HSV1-UL30-440 | − | GCGGCCCTGCGCGAGTCCCC | 20 | 3378 |
| HSV1-UL30-441 | − | TCCCCGGGCGCGTCGTTCCG | 20 | 3379 |
| HSV1-UL30-442 | − | GTCGTTCCGCGGCATCTCCG | 20 | 3380 |
| HSV1-UL30-443 | − | CATCTCCGCGGACCACTTCG | 20 | 3381 |
| HSV1-UL30-444 | − | CTCCGCGGACCACTTCGAGG | 20 | 3382 |
| HSV1-UL30-445 | − | CGCGGACCACTTCGAGGCGG | 20 | 3383 |
| HSV1-UL30-446 | − | GGACCACTTCGAGGCGGAGG | 20 | 3384 |
| HSV1-UL30-447 | − | CCACTTCGAGGCGGAGGTGG | 20 | 3385 |
| HSV1-UL30-448 | − | TACCGCGTCTACGTCCGAAG | 20 | 3386 |
| HSV1-UL30-449 | − | ACCGCGTCTACGTCCGAAGC | 20 | 3387 |
| HSV1-UL30-450 | − | CCTGTGCGACAACTTCTGCC | 20 | 3388 |
| HSV1-UL30-451 | − | CCCGGCCATCAAGAAGTACG | 20 | 3389 |
| HSV1-UL30-452 | − | CCGGCCATCAAGAAGTACGA | 20 | 3390 |
| HSV1-UL30-453 | − | GCCATCAAGAAGTACGAGGG | 20 | 3391 |
| HSV1-UL30-454 | − | CCATCAAGAAGTACGAGGGT | 20 | 3392 |
| HSV1-UL30-455 | − | CATCAAGAAGTACGAGGGTG | 20 | 3393 |
| HSV1-UL30-456 | − | GTGGGGTCGACGCCACCACC | 20 | 3394 |
| HSV1-UL30-457 | − | CGCCACCACCCGGTTCATCC | 20 | 3395 |
| HSV1-UL30-458 | − | CGGTTCATCCTGGACAACCC | 20 | 3396 |
| HSV1-UL30-459 | − | GGTTCATCCTGGACAACCCC | 20 | 3397 |
| HSV1-UL30-460 | − | AACCCCGGGTTCGTCACCTT | 20 | 3398 |
| HSV1-UL30-461 | − | CCGGGTTCGTCACCTTCGGC | 20 | 3399 |
| HSV1-UL30-462 | − | CGGCTGGTACCGTCTCAAAC | 20 | 3400 |
| HSV1-UL30-463 | − | GGCTGGTACCGTCTCAAACC | 20 | 3401 |
| HSV1-UL30-464 | − | GGTACCGTCTCAAACCGGGC | 20 | 3402 |
| HSV1-UL30-465 | − | GAACAACACGCTAGCCCAGC | 20 | 3403 |
| HSV1-UL30-466 | − | CAACACGCTAGCCCAGCCGG | 20 | 3404 |
| HSV1-UL30-467 | − | AGCCCAGCCGGCGGCCCCGA | 20 | 3405 |
| HSV1-UL30-468 | − | CCGGCGGCCCCGATGGCCTT | 20 | 3406 |
| HSV1-UL30-469 | − | CGGCGGCCCCGATGGCCTTC | 20 | 3407 |
| HSV1-UL30-470 | − | CGACGTCGAGTTTAACTGTA | 20 | 3408 |
| HSV1-UL30-471 | − | CGTCGAGTTTAACTGTACGG | 20 | 3409 |
| HSV1-UL30-472 | − | TAACTGTACGGCGGACAACC | 20 | 3410 |
| HSV1-UL30-473 | − | GGCGGACAACCTGGCCATCG | 20 | 3411 |
| HSV1-UL30-474 | − | GCGGACAACCTGGCCATCGA | 20 | 3412 |
| HSV1-UL30-475 | − | CGGACAACCTGGCCATCGAG | 20 | 3413 |
| HSV1-UL30-476 | − | GGACAACCTGGCCATCGAGG | 20 | 3414 |
| HSV1-UL30-477 | − | GACAACCTGGCCATCGAGGG | 20 | 3415 |
| HSV1-UL30-478 | − | GGGGGGCATGAGCGACCTAC | 20 | 3416 |
| HSV1-UL30-479 | − | GTGCTTCGATATCGAATGCA | 20 | 3417 |
| HSV1-UL30-480 | − | CTTCGATATCGAATGCAAGG | 20 | 3418 |
| HSV1-UL30-481 | − | TTCGATATCGAATGCAAGGC | 20 | 3419 |
| HSV1-UL30-482 | − | TCGATATCGAATGCAAGGCG | 20 | 3420 |
| HSV1-UL30-483 | − | CGATATCGAATGCAAGGCGG | 20 | 3421 |
| HSV1-UL30-484 | − | GATATCGAATGCAAGGCGGG | 20 | 3422 |
| HSV1-UL30-485 | − | ATATCGAATGCAAGGCGGGG | 20 | 3423 |
| HSV1-UL30-486 | − | TATCGAATGCAAGGCGGGGG | 20 | 3424 |
| HSV1-UL30-487 | − | CGAATGCAAGGCGGGGGGGG | 20 | 3425 |
| HSV1-UL30-488 | − | GGCGGGGGGGGAGGACGAGC | 20 | 3426 |
| HSV1-UL30-489 | − | GGAGGACGAGCTGGCCTTTC | 20 | 3427 |
| HSV1-UL30-490 | − | GGACGAGCTGGCCTTTCCGG | 20 | 3428 |
| HSV1-UL30-491 | − | GAGCTGGCCTTTCCGGTGGC | 20 | 3429 |
| HSV1-UL30-492 | − | AGCTGGCCTTTCCGGTGGCC | 20 | 3430 |
| HSV1-UL30-493 | − | CTTTCCGGTGGCCGGGCACC | 20 | 3431 |
| HSV1-UL30-494 | − | TCCGGTGGCCGGGCACCCGG | 20 | 3432 |
| HSV1-UL30-495 | − | GGCCGGGCACCCGGAGGACC | 20 | 3433 |
| HSV1-UL30-496 | − | CGACCTGTCCACCACCGCCC | 20 | 3434 |
| HSV1-UL30-497 | − | CACGTCCTCCTGTTTTCGCT | 20 | 3435 |
| HSV1-UL30-498 | − | CGAATCCCACCTGAACGAGC | 20 | 3436 |
| HSV1-UL30-499 | − | ATCCCACCTGAACGAGCTGG | 20 | 3437 |
| HSV1-UL30-500 | − | ACCTGAACGAGCTGGCGGCC | 20 | 3438 |
| HSV1-UL30-501 | − | CCTGAACGAGCTGGCGGCCA | 20 | 3439 |
| HSV1-UL30-502 | − | CTGAACGAGCTGGCGGCCAG | 20 | 3440 |
| HSV1-UL30-503 | − | CAGGGGCCTGCCCACGCCCG | 20 | 3441 |
| HSV1-UL30-504 | − | CCTGCCCACGCCCGTGGTTC | 20 | 3442 |
| HSV1-UL30-505 | − | CAGCGAATTCGAGATGCTGT | 20 | 3443 |
| HSV1-UL30-506 | − | ATGACCCTTGTGAAACAGTA | 20 | 3444 |
| HSV1-UL30-507 | − | TACGGCCCCGAGTTCGTGAC | 20 | 3445 |
| HSV1-UL30-508 | − | ACGGCCCCGAGTTCGTGACC | 20 | 3446 |
| HSV1-UL30-509 | − | ACAACATCATCAACTTCGAC | 20 | 3447 |
| HSV1-UL30-510 | − | CTTCGACTGGCCCTTCTTGC | 20 | 3448 |
| HSV1-UL30-511 | − | CTTCTTGCTGGCCAAGCTGA | 20 | 3449 |
| HSV1-UL30-512 | − | CAAGCTGACGGACATTTACA | 20 | 3450 |
| HSV1-UL30-513 | − | GGACATTTACAAGGTCCCCC | 20 | 3451 |
| HSV1-UL30-514 | − | ATTTACAAGGTCCCCCTGGA | 20 | 3452 |
| HSV1-UL30-515 | − | TTTACAAGGTCCCCCTGGAC | 20 | 3453 |
| HSV1-UL30-516 | − | AAGGTCCCCCTGGACGGGTA | 20 | 3454 |
| HSV1-UL30-517 | − | GACGGGTACGGCCGCATGAA | 20 | 3455 |
| HSV1-UL30-518 | − | GGTACGGCCGCATGAACGGC | 20 | 3456 |
| HSV1-UL30-519 | − | GTACGGCCGCATGAACGGCC | 20 | 3457 |
| HSV1-UL30-520 | − | TACGGCCGCATGAACGGCCG | 20 | 3458 |
| HSV1-UL30-521 | − | GCCGGGGCGTGTTTCGCGTG | 20 | 3459 |
| HSV1-UL30-522 | − | CCGGGGCGTGTTTCGCGTGT | 20 | 3460 |
| HSV1-UL30-523 | − | GTGTTTCGCGTGTGGGACAT | 20 | 3461 |
| HSV1-UL30-524 | − | CCAGAAGCGCAGCAAGATAA | 20 | 3462 |
| HSV1-UL30-525 | − | CGCAGCAAGATAAAGGTGAA | 20 | 3463 |
| HSV1-UL30-526 | − | CAAGATAAAGGTGAACGGCA | 20 | 3464 |
| HSV1-UL30-527 | − | ATGGTGAACATCGACATGTA | 20 | 3465 |
| HSV1-UL30-528 | − | TGGTGAACATCGACATGTAC | 20 | 3466 |
| HSV1-UL30-529 | − | GAGCTACAAGCTCAACGCCG | 20 | 3467 |
| HSV1-UL30-530 | − | CGTGGCCGAAGCCGTCCTGA | 20 | 3468 |
| HSV1-UL30-531 | − | CGTCCTGAAGGACAAGAAGA | 20 | 3469 |
| HSV1-UL30-532 | − | ATCCCCGCCTACTACGCCGC | 20 | 3470 |
| HSV1-UL30-533 | − | TCCCCGCCTACTACGCCGCC | 20 | 3471 |
| HSV1-UL30-534 | − | GCCGCCGGGCCCGCGCAACG | 20 | 3472 |
| HSV1-UL30-535 | − | CCGCCGGGCCCGCGCAACGC | 20 | 3473 |
| HSV1-UL30-536 | − | CGCCGGGCCCGCGCAACGCG | 20 | 3474 |
| HSV1-UL30-537 | − | CCCGCGCAACGCGGGGTGAT | 20 | 3475 |
| HSV1-UL30-538 | − | GATCGGCGAGTACTGCATAC | 20 | 3476 |
| HSV1-UL30-539 | − | CTGCATACAGGATTCCCTGC | 20 | 3477 |
| HSV1-UL30-540 | − | CATACAGGATTCCCTGCTGG | 20 | 3478 |
| HSV1-UL30-541 | − | ATACAGGATTCCCTGCTGGT | 20 | 3479 |
| HSV1-UL30-542 | − | TTTTAAGTTTTTGCCCCATC | 20 | 3480 |
| HSV1-UL30-543 | − | TTTGCCCCATCTGGAGCTCT | 20 | 3481 |
| HSV1-UL30-544 | − | GCTCTCGGCCGTCGCGCGCT | 20 | 3482 |
| HSV1-UL30-545 | − | CTCGGCCGTCGCGCGCTTGG | 20 | 3483 |
| HSV1-UL30-546 | − | TCGGCCGTCGCGCGCTTGGC | 20 | 3484 |
| HSV1-UL30-547 | − | ATCACCCGCACCATCTACGA | 20 | 3485 |
| HSV1-UL30-548 | − | CTTTACGTGCCTGCTGCGCC | 20 | 3486 |
| HSV1-UL30-549 | − | GCTGCGCCTGGCCGACCAGA | 20 | 3487 |
| HSV1-UL30-550 | − | CTGCGCCTGGCCGACCAGAA | 20 | 3488 |
| HSV1-UL30-551 | − | CCAGAAGGGCTTTATTCTGC | 20 | 3489 |
| HSV1-UL30-552 | − | CTTTATTCTGCCGGACACCC | 20 | 3490 |
| HSV1-UL30-553 | − | TTTATTCTGCCGGACACCCA | 20 | 3491 |
| HSV1-UL30-554 | − | TTATTCTGCCGGACACCCAG | 20 | 3492 |
| HSV1-UL30-555 | − | CGGACACCCAGGGGCGATTT | 20 | 3493 |
| HSV1-UL30-556 | − | GGACACCCAGGGGCGATTTA | 20 | 3494 |
| HSV1-UL30-557 | − | GACACCCAGGGGCGATTTAG | 20 | 3495 |
| HSV1-UL30-558 | − | CAGGGGCGATTTAGGGGCGC | 20 | 3496 |
| HSV1-UL30-559 | − | AGGGGCGATTTAGGGGCGCC | 20 | 3497 |
| HSV1-UL30-560 | − | GGGGCGATTTAGGGGCGCCG | 20 | 3498 |
| HSV1-UL30-561 | − | GGGCGATTTAGGGGCGCCGG | 20 | 3499 |
| HSV1-UL30-562 | − | GGCGATTTAGGGGCGCCGGG | 20 | 3500 |
| HSV1-UL30-563 | − | GCGATTTAGGGGCGCCGGGG | 20 | 3501 |
| HSV1-UL30-564 | − | ATTTAGGGGCGCCGGGGGGG | 20 | 3502 |
| HSV1-UL30-565 | − | GGGGGAGGCGCCCAAGCGTC | 20 | 3503 |
| HSV1-UL30-566 | − | CCAAGCGTCCGGCCGCAGCC | 20 | 3504 |
| HSV1-UL30-567 | − | CAAGCGTCCGGCCGCAGCCC | 20 | 3505 |
| HSV1-UL30-568 | − | GCGTCCGGCCGCAGCCCGGG | 20 | 3506 |
| HSV1-UL30-569 | − | GGCCGCAGCCCGGGAGGACG | 20 | 3507 |
| HSV1-UL30-570 | − | CAGCCCGGGAGGACGAGGAG | 20 | 3508 |
| HSV1-UL30-571 | − | GGAGGACGAGGAGCGGCCAG | 20 | 3509 |
| HSV1-UL30-572 | − | GGACGAGGAGCGGCCAGAGG | 20 | 3510 |
| HSV1-UL30-573 | − | CGAGGAGCGGCCAGAGGAGG | 20 | 3511 |
| HSV1-UL30-574 | − | GAGGAGCGGCCAGAGGAGGA | 20 | 3512 |
| HSV1-UL30-575 | − | AGGAGCGGCCAGAGGAGGAG | 20 | 3513 |
| HSV1-UL30-576 | − | GGAGCGGCCAGAGGAGGAGG | 20 | 3514 |
| HSV1-UL30-577 | − | GCGGCCAGAGGAGGAGGGGG | 20 | 3515 |
| HSV1-UL30-578 | − | AGAGGAGGAGGGGGAGGACG | 20 | 3516 |
| HSV1-UL30-579 | − | GGAGGACGAGGACGAACGCG | 20 | 3517 |
| HSV1-UL30-580 | − | GGACGAGGACGAACGCGAGG | 20 | 3518 |
| HSV1-UL30-581 | − | GACGAGGACGAACGCGAGGA | 20 | 3519 |
| HSV1-UL30-582 | − | GAGGACGAACGCGAGGAGGG | 20 | 3520 |
| HSV1-UL30-583 | − | AGGACGAACGCGAGGAGGGC | 20 | 3521 |
| HSV1-UL30-584 | − | GGACGAACGCGAGGAGGGCG | 20 | 3522 |
| HSV1-UL30-585 | − | GACGAACGCGAGGAGGGCGG | 20 | 3523 |
| HSV1-UL30-586 | − | GCGAGGAGGGCGGGGGCGAG | 20 | 3524 |
| HSV1-UL30-587 | − | CGAGGAGGGCGGGGGCGAGC | 20 | 3525 |
| HSV1-UL30-588 | − | GGGCGGGGGCGAGCGGGAGC | 20 | 3526 |
| HSV1-UL30-589 | − | CGGGGGCGAGCGGGAGCCGG | 20 | 3527 |
| HSV1-UL30-590 | − | GGGGGCGAGCGGGAGCCGGA | 20 | 3528 |
| HSV1-UL30-591 | − | AGCGGGAGCCGGAGGGCGCG | 20 | 3529 |
| HSV1-UL30-592 | − | GCGGGAGCCGGAGGGCGCGC | 20 | 3530 |
| HSV1-UL30-593 | − | GAGGGCGCGCGGGAGACCGC | 20 | 3531 |
| HSV1-UL30-594 | − | GCGCGCGGGAGACCGCCGGC | 20 | 3532 |
| HSV1-UL30-595 | − | GGAGACCGCCGGCAGGCACG | 20 | 3533 |
| HSV1-UL30-596 | − | GAGACCGCCGGCAGGCACGT | 20 | 3534 |
| HSV1-UL30-597 | − | AGACCGCCGGCAGGCACGTG | 20 | 3535 |
| HSV1-UL30-598 | − | CGGCAGGCACGTGGGGTACC | 20 | 3536 |
| HSV1-UL30-599 | − | GGCAGGCACGTGGGGTACCA | 20 | 3537 |
| HSV1-UL30-600 | − | GCAGGCACGTGGGGTACCAG | 20 | 3538 |
| HSV1-UL30-601 | − | CAGGCACGTGGGGTACCAGG | 20 | 3539 |
| HSV1-UL30-602 | − | ACGTGGGGTACCAGGGGGCC | 20 | 3540 |
| HSV1-UL30-603 | − | CGTGGGGTACCAGGGGGCCA | 20 | 3541 |
| HSV1-UL30-604 | − | AGGGTCCTTGACCCCACTTC | 20 | 3542 |
| HSV1-UL30-605 | − | GGGTCCTTGACCCCACTTCC | 20 | 3543 |
| HSV1-UL30-606 | − | CGGGTTTCACGTGAACCCCG | 20 | 3544 |
| HSV1-UL30-607 | − | GTTTCACGTGAACCCCGTGG | 20 | 3545 |
| HSV1-UL30-608 | − | CCTGTACCCCAGCATCATCC | 20 | 3546 |
| HSV1-UL30-609 | − | GCTTCAGCACGCTCTCCCTG | 20 | 3547 |
| HSV1-UL30-610 | − | CTTCAGCACGCTCTCCCTGA | 20 | 3548 |
| HSV1-UL30-611 | − | CTCCCTGAGGGCCGACGCAG | 20 | 3549 |
| HSV1-UL30-612 | − | GGCCGACGCAGTGGCGCACC | 20 | 3550 |
| HSV1-UL30-613 | − | CGACGCAGTGGCGCACCTGG | 20 | 3551 |
| HSV1-UL30-614 | − | CGCAGTGGCGCACCTGGAGG | 20 | 3552 |
| HSV1-UL30-615 | − | GCAGTGGCGCACCTGGAGGC | 20 | 3553 |
| HSV1-UL30-616 | − | GGCGCACCTGGAGGCGGGCA | 20 | 3554 |
| HSV1-UL30-617 | − | GGAGGCGGGCAAGGACTACC | 20 | 3555 |
| HSV1-UL30-618 | − | CAAGGACTACCTGGAGATCG | 20 | 3556 |
| HSV1-UL30-619 | − | GGACTACCTGGAGATCGAGG | 20 | 3557 |
| HSV1-UL30-620 | − | GACTACCTGGAGATCGAGGT | 20 | 3558 |
| HSV1-UL30-621 | − | ACTACCTGGAGATCGAGGTG | 20 | 3559 |
| HSV1-UL30-622 | − | CTACCTGGAGATCGAGGTGG | 20 | 3560 |
| HSV1-UL30-623 | − | TACCTGGAGATCGAGGTGGG | 20 | 3561 |
| HSV1-UL30-624 | − | ACCTGGAGATCGAGGTGGGG | 20 | 3562 |
| HSV1-UL30-625 | − | AGATCGAGGTGGGGGGGCGA | 20 | 3563 |
| HSV1-UL30-626 | − | GCGACGGCTGTTCTTCGTCA | 20 | 3564 |
| HSV1-UL30-627 | − | GCCTCCTCAGCATCCTCCTG | 20 | 3565 |
| HSV1-UL30-628 | − | CCTCCTCAGCATCCTCCTGC | 20 | 3566 |
| HSV1-UL30-629 | − | TCAGCATCCTCCTGCGGGAC | 20 | 3567 |
| HSV1-UL30-630 | − | TGCGAAAGCAGATCCGCTCG | 20 | 3568 |
| HSV1-UL30-631 | − | GATTCCCCAGAGCAGCCCCG | 20 | 3569 |
| HSV1-UL30-632 | − | TCCCCAGAGCAGCCCCGAGG | 20 | 3570 |
| HSV1-UL30-633 | − | CCCCGAGGAGGCCGTGCTCC | 20 | 3571 |
| HSV1-UL30-634 | − | CGTGCTCCTGGACAAGCAGC | 20 | 3572 |
| HSV1-UL30-635 | − | CAAGCAGCAGGCCGCCATCA | 20 | 3573 |
| HSV1-UL30-636 | − | CATCAAGGTCGTGTGTAACT | 20 | 3574 |
| HSV1-UL30-637 | − | GTCGTGTGTAACTCGGTGTA | 20 | 3575 |
| HSV1-UL30-638 | − | TCGTGTGTAACTCGGTGTAC | 20 | 3576 |
| HSV1-UL30-639 | − | TAACTCGGTGTACGGGTTCA | 20 | 3577 |
| HSV1-UL30-640 | − | AACTCGGTGTACGGGTTCAC | 20 | 3578 |
| HSV1-UL30-641 | − | GGGTTCACGGGAGTGCAGCA | 20 | 3579 |
| HSV1-UL30-642 | − | GTGCCTGCACGTTGCCGCGA | 20 | 3580 |
| HSV1-UL30-643 | − | GCCGCGACGGTGACGACCAT | 20 | 3581 |
| HSV1-UL30-644 | − | GCGAGTACGTCCACGCGCGC | 20 | 3582 |
| HSV1-UL30-645 | − | CGAGTACGTCCACGCGCGCT | 20 | 3583 |
| HSV1-UL30-646 | − | GTACGTCCACGCGCGCTGGG | 20 | 3584 |
| HSV1-UL30-647 | − | GGCGGCCTTCGAACAGCTCC | 20 | 3585 |
| HSV1-UL30-648 | − | ACAGCTCCTGGCCGATTTCC | 20 | 3586 |
| HSV1-UL30-649 | − | GCTCCTGGCCGATTTCCCGG | 20 | 3587 |
| HSV1-UL30-650 | − | CCTGGCCGATTTCCCGGAGG | 20 | 3588 |
| HSV1-UL30-651 | − | GCGGCCGACATGCGCGCCCC | 20 | 3589 |
| HSV1-UL30-652 | − | CGGCCGACATGCGCGCCCCC | 20 | 3590 |
| HSV1-UL30-653 | − | TATTCCATGCGCATCATCTA | 20 | 3591 |
| HSV1-UL30-654 | − | ATTCCATGCGCATCATCTAC | 20 | 3592 |
| HSV1-UL30-655 | − | TTCCATGCGCATCATCTACG | 20 | 3593 |
| HSV1-UL30-656 | − | GCGCATCATCTACGGGGACA | 20 | 3594 |
| HSV1-UL30-657 | − | TCCATCTTTGTGCTGTGCCG | 20 | 3595 |
| HSV1-UL30-658 | − | TGTGCTGTGCCGCGGCCTCA | 20 | 3596 |
| HSV1-UL30-659 | − | TGCCGCGGCCTCACGGCCGC | 20 | 3597 |
| HSV1-UL30-660 | − | GCCGCGGCCTCACGGCCGCC | 20 | 3598 |
| HSV1-UL30-661 | − | CCTCACGGCCGCCGGGCTGA | 20 | 3599 |
| HSV1-UL30-662 | − | GGCCGCCGGGCTGACGGCCG | 20 | 3600 |
| HSV1-UL30-663 | − | GCCGCCGGGCTGACGGCCGT | 20 | 3601 |
| HSV1-UL30-664 | − | GACGGCCGTGGGCGACAAGA | 20 | 3602 |
| HSV1-UL30-665 | − | ATCGCCAAGAAAAAGTACAT | 20 | 3603 |
| HSV1-UL30-666 | − | AAGTACATCGGCGTCATCTA | 20 | 3604 |
| HSV1-UL30-667 | − | AGTACATCGGCGTCATCTAC | 20 | 3605 |
| HSV1-UL30-668 | − | GTACATCGGCGTCATCTACG | 20 | 3606 |
| HSV1-UL30-669 | − | TACATCGGCGTCATCTACGG | 20 | 3607 |
| HSV1-UL30-670 | − | CGGGGGTAAGATGCTCATCA | 20 | 3608 |
| HSV1-UL30-671 | − | GGGGGTAAGATGCTCATCAA | 20 | 3609 |
| HSV1-UL30-672 | − | TAAGATGCTCATCAAGGGCG | 20 | 3610 |
| HSV1-UL30-673 | − | GCTCATCAAGGGCGTGGATC | 20 | 3611 |
| HSV1-UL30-674 | − | CGTTTATCAACCGCACCTCC | 20 | 3612 |
| HSV1-UL30-675 | − | GTTTATCAACCGCACCTCCA | 20 | 3613 |
| HSV1-UL30-676 | − | CAACCGCACCTCCAGGGCCC | 20 | 3614 |
| HSV1-UL30-677 | − | TTTTACGACGATACCGTCTC | 20 | 3615 |
| HSV1-UL30-678 | − | CGACGATACCGTCTCCGGAG | 20 | 3616 |
| HSV1-UL30-679 | − | CGCGTTAGCCGAGCGCCCCG | 20 | 3617 |
| HSV1-UL30-680 | − | GTTAGCCGAGCGCCCCGCGG | 20 | 3618 |
| HSV1-UL30-681 | − | CCGAGCGCCCCGCGGAGGAG | 20 | 3619 |
| HSV1-UL30-682 | − | GCGCCCCGCGGAGGAGTGGC | 20 | 3620 |
| HSV1-UL30-683 | − | GCTGGCGCGACCCCTGCCCG | 20 | 3621 |
| HSV1-UL30-684 | − | CTGGCGCGACCCCTGCCCGA | 20 | 3622 |
| HSV1-UL30-685 | − | ACCCCTGCCCGAGGGACTGC | 20 | 3623 |
| HSV1-UL30-686 | − | CCCGAGGGACTGCAGGCGTT | 20 | 3624 |
| HSV1-UL30-687 | − | CCGAGGGACTGCAGGCGTTC | 20 | 3625 |
| HSV1-UL30-688 | − | CGAGGGACTGCAGGCGTTCG | 20 | 3626 |
| HSV1-UL30-689 | − | CCGTCCTCGTAGACGCCCAT | 20 | 3627 |
| HSV1-UL30-690 | − | CCATCGGCGCATCACCGACC | 20 | 3628 |
| HSV1-UL30-691 | − | GGCGCATCACCGACCCGGAG | 20 | 3629 |
| HSV1-UL30-692 | − | GCGCATCACCGACCCGGAGA | 20 | 3630 |
| HSV1-UL30-693 | − | CGACCCGGAGAGGGACATCC | 20 | 3631 |
| HSV1-UL30-694 | − | CGCGTACACCAACAAGCGCC | 20 | 3632 |
| HSV1-UL30-695 | − | CAAGCGCCTGGCCCACCTGA | 20 | 3633 |
| HSV1-UL30-696 | − | GACGGTGTATTACAAGCTCA | 20 | 3634 |
| HSV1-UL30-697 | − | GCTCATGGCCCGCCGCGCGC | 20 | 3635 |
| HSV1-UL30-698 | − | CGCGCAGGTCCCGTCCATCA | 20 | 3636 |
| HSV1-UL30-699 | − | AGGTCCCGTCCATCAAGGAC | 20 | 3637 |
| HSV1-UL30-700 | − | CCGGATCCCGTACGTGATCG | 20 | 3638 |
| HSV1-UL30-701 | − | GATCGTGGCCCAGACCCGCG | 20 | 3639 |
| HSV1-UL30-702 | − | GGCCCAGACCCGCGAGGTAG | 20 | 3640 |
| HSV1-UL30-703 | − | GACCCGCGAGGTAGAGGAGA | 20 | 3641 |
| HSV1-UL30-704 | − | AGGTAGAGGAGACGGTCGCG | 20 | 3642 |
| HSV1-UL30-705 | − | AGAGGAGACGGTCGCGCGGC | 20 | 3643 |
| HSV1-UL30-706 | − | GAGCTAGACGCCGCCGCCCC | 20 | 3644 |
| HSV1-UL30-707 | − | AGCTAGACGCCGCCGCCCCA | 20 | 3645 |
| HSV1-UL30-708 | − | GCTAGACGCCGCCGCCCCAG | 20 | 3646 |
| HSV1-UL30-709 | − | GGACGAGCCCGCCCCCCCCG | 20 | 3647 |
| HSV1-UL30-710 | − | CCCCGCGGCCCTGCCCTCCC | 20 | 3648 |
| HSV1-UL30-711 | − | CCTCCCCGGCCAAGCGCCCC | 20 | 3649 |
| HSV1-UL30-712 | − | CTCCCCGGCCAAGCGCCCCC | 20 | 3650 |
| HSV1-UL30-713 | − | GCCGTCGCCTGCCGACCCCC | 20 | 3651 |
| HSV1-UL30-714 | − | CCGTCGCCTGCCGACCCCCC | 20 | 3652 |
| HSV1-UL30-715 | − | TCGCCTGCCGACCCCCCGGG | 20 | 3653 |
| HSV1-UL30-716 | − | GTCCAAGCCCCGCAAGCTGC | 20 | 3654 |
| HSV1-UL30-717 | − | CAAGCTGCTGGTGTCCGAGC | 20 | 3655 |
| HSV1-UL30-718 | − | GCTGGTGTCCGAGCTGGCCG | 20 | 3656 |
| HSV1-UL30-719 | − | CCCGCATACGCCATTGCCCA | 20 | 3657 |
| HSV1-UL30-720 | − | CCACGGCGTCGCCCTGAACA | 20 | 3658 |
| HSV1-UL30-721 | − | CTATTACTTCTCCCACCTGT | 20 | 3659 |
| HSV1-UL30-722 | − | TATTACTTCTCCCACCTGTT | 20 | 3660 |
| HSV1-UL30-723 | − | ATTACTTCTCCCACCTGTTG | 20 | 3661 |
| HSV1-UL30-724 | − | TTACTTCTCCCACCTGTTGG | 20 | 3662 |
| HSV1-UL30-725 | − | CTTCTCCCACCTGTTGGGGG | 20 | 3663 |
| HSV1-UL30-726 | − | GGCGGCGTGCGTGACATTCA | 20 | 3664 |
| HSV1-UL30-727 | − | GTGACATTCAAGGCCCTGTT | 20 | 3665 |
| HSV1-UL30-728 | − | TGACATTCAAGGCCCTGTTT | 20 | 3666 |
| HSV1-UL30-729 | − | TCACCGAGAGTCTGTTAAAA | 20 | 3667 |
| HSV1-UL30-730 | − | AAAGGTTTATTCCCGAAGTG | 20 | 3668 |
| HSV1-UL30-731 | − | TCCCGAAGTGTGGCACCCCC | 20 | 3669 |
| HSV1-UL30-732 | − | GTGGCACCCCCCGGACGACG | 20 | 3670 |
| HSV1-UL30-733 | − | CCCCGGACGACGTGGCCGCG | 20 | 3671 |
| HSV1-UL30-734 | − | ACGACGTGGCCGCGCGGCTC | 20 | 3672 |
| HSV1-UL30-735 | − | GCCGCGCGGCTCCGGACCGC | 20 | 3673 |
| HSV1-UL30-736 | − | CCGCGCGGCTCCGGACCGCA | 20 | 3674 |
| HSV1-UL30-737 | − | CGGCTCCGGACCGCAGGGTT | 20 | 3675 |
| HSV1-UL30-738 | − | GGCTCCGGACCGCAGGGTTC | 20 | 3676 |
| HSV1-UL30-739 | − | GCTCCGGACCGCAGGGTTCG | 20 | 3677 |
| HSV1-UL30-740 | − | CCGGACCGCAGGGTTCGGGG | 20 | 3678 |
| HSV1-UL30-741 | − | GACCGCAGGGTTCGGGGCGG | 20 | 3679 |
| HSV1-UL30-742 | − | ACCGCAGGGTTCGGGGCGGT | 20 | 3680 |
| HSV1-UL30-743 | − | GGGTTCGGGGCGGTGGGTGC | 20 | 3681 |
| HSV1-UL30-744 | − | GGCGGTGGGTGCCGGCGCTA | 20 | 3682 |
| HSV1-UL30-745 | − | GGTGGGTGCCGGCGCTACGG | 20 | 3683 |
| HSV1-UL30-746 | − | GGGTGCCGGCGCTACGGCGG | 20 | 3684 |
| HSV1-UL30-747 | − | GGACGCCATCACACCGA | 17 | 3685 |
| HSV1-UL30-748 | − | GACGCCATCACACCGAC | 17 | 3686 |
| HSV1-UL30-749 | − | ACGCCATCACACCGACG | 17 | 3687 |
| HSV1-UL30-750 | − | GACCGTCATCACGCTCC | 17 | 3688 |
| HSV1-UL30-751 | − | ACCGTCATCACGCTCCT | 17 | 3689 |
| HSV1-UL30-752 | − | GCTCCTGGGCCTGACTC | 17 | 3690 |
| HSV1-UL30-753 | − | CTGGGCCTGACTCCGGA | 17 | 3691 |
| HSV1-UL30-754 | − | TGACTCCGGAAGGCCAC | 17 | 3692 |
| HSV1-UL30-755 | − | GACTCCGGAAGGCCACC | 17 | 3693 |
| HSV1-UL30-756 | − | TCCGGAAGGCCACCGGG | 17 | 3694 |
| HSV1-UL30-757 | − | GTGGCCGTTCACGTTTA | 17 | 3695 |
| HSV1-UL30-758 | − | TTCACGTTTACGGCACG | 17 | 3696 |
| HSV1-UL30-759 | − | GTACTTTTACATGAACA | 17 | 3697 |
| HSV1-UL30-760 | − | CTTTTACATGAACAAGG | 17 | 3698 |
| HSV1-UL30-761 | − | TTACATGAACAAGGAGG | 17 | 3699 |
| HSV1-UL30-762 | − | ACAAGGAGGAGGTCGAC | 17 | 3700 |
| HSV1-UL30-763 | − | AGATCTCTGCGAGCGCA | 17 | 3701 |
| HSV1-UL30-764 | − | CTGCGAGCGCATGGCCG | 17 | 3702 |
| HSV1-UL30-765 | − | GGCCCTGCGCGAGTCCC | 17 | 3703 |
| HSV1-UL30-766 | − | GCCCTGCGCGAGTCCCC | 17 | 3704 |
| HSV1-UL30-767 | − | CCGGGCGCGTCGTTCCG | 17 | 3705 |
| HSV1-UL30-768 | − | GTTCCGCGGCATCTCCG | 17 | 3706 |
| HSV1-UL30-769 | − | CTCCGCGGACCACTTCG | 17 | 3707 |
| HSV1-UL30-770 | − | CGCGGACCACTTCGAGG | 17 | 3708 |
| HSV1-UL30-771 | − | GGACCACTTCGAGGCGG | 17 | 3709 |
| HSV1-UL30-772 | − | CCACTTCGAGGCGGAGG | 17 | 3710 |
| HSV1-UL30-773 | − | CTTCGAGGCGGAGGTGG | 17 | 3711 |
| HSV1-UL30-774 | − | CGCGTCTACGTCCGAAG | 17 | 3712 |
| HSV1-UL30-775 | − | GCGTCTACGTCCGAAGC | 17 | 3713 |
| HSV1-UL30-776 | − | GTGCGACAACTTCTGCC | 17 | 3714 |
| HSV1-UL30-777 | − | GGCCATCAAGAAGTACG | 17 | 3715 |
| HSV1-UL30-778 | − | GCCATCAAGAAGTACGA | 17 | 3716 |
| HSV1-UL30-779 | − | ATCAAGAAGTACGAGGG | 17 | 3717 |
| HSV1-UL30-780 | − | TCAAGAAGTACGAGGGT | 17 | 3718 |
| HSV1-UL30-781 | − | CAAGAAGTACGAGGGTG | 17 | 3719 |
| HSV1-UL30-782 | − | GGGTCGACGCCACCACC | 17 | 3720 |
| HSV1-UL30-783 | − | CACCACCCGGTTCATCC | 17 | 3721 |
| HSV1-UL30-784 | − | TTCATCCTGGACAACCC | 17 | 3722 |
| HSV1-UL30-785 | − | TCATCCTGGACAACCCC | 17 | 3723 |
| HSV1-UL30-786 | − | CCCGGGTTCGTCACCTT | 17 | 3724 |
| HSV1-UL30-787 | − | GGTTCGTCACCTTCGGC | 17 | 3725 |
| HSV1-UL30-788 | − | CTGGTACCGTCTCAAAC | 17 | 3726 |
| HSV1-UL30-789 | − | TGGTACCGTCTCAAACC | 17 | 3727 |
| HSV1-UL30-790 | − | ACCGTCTCAAACCGGGC | 17 | 3728 |
| HSV1-UL30-791 | − | CAACACGCTAGCCCAGC | 17 | 3729 |
| HSV1-UL30-792 | − | CACGCTAGCCCAGCCGG | 17 | 3730 |
| HSV1-UL30-793 | − | CCAGCCGGCGGCCCCGA | 17 | 3731 |
| HSV1-UL30-794 | − | GCGGCCCCGATGGCCTT | 17 | 3732 |
| HSV1-UL30-795 | − | CGGCCCCGATGGCCTTC | 17 | 3733 |
| HSV1-UL30-796 | − | CGTCGAGTTTAACTGTA | 17 | 3734 |
| HSV1-UL30-797 | − | CGAGTTTAACTGTACGG | 17 | 3735 |
| HSV1-UL30-798 | − | CTGTACGGCGGACAACC | 17 | 3736 |
| HSV1-UL30-799 | − | GGACAACCTGGCCATCG | 17 | 3737 |
| HSV1-UL30-800 | − | GACAACCTGGCCATCGA | 17 | 3738 |
| HSV1-UL30-801 | − | ACAACCTGGCCATCGAG | 17 | 3739 |
| HSV1-UL30-802 | − | CAACCTGGCCATCGAGG | 17 | 3740 |
| HSV1-UL30-803 | − | AACCTGGCCATCGAGGG | 17 | 3741 |
| HSV1-UL30-804 | − | GGGCATGAGCGACCTAC | 17 | 3742 |
| HSV1-UL30-805 | − | CTTCGATATCGAATGCA | 17 | 3743 |
| HSV1-UL30-806 | − | CGATATCGAATGCAAGG | 17 | 3744 |
| HSV1-UL30-807 | − | GATATCGAATGCAAGGC | 17 | 3745 |
| HSV1-UL30-808 | − | ATATCGAATGCAAGGCG | 17 | 3746 |
| HSV1-UL30-809 | − | TATCGAATGCAAGGCGG | 17 | 3747 |
| HSV1-UL30-810 | − | ATCGAATGCAAGGCGGG | 17 | 3748 |
| HSV1-UL30-811 | − | TCGAATGCAAGGCGGGG | 17 | 3749 |
| HSV1-UL30-812 | − | CGAATGCAAGGCGGGGG | 17 | 3750 |
| HSV1-UL30-813 | − | ATGCAAGGCGGGGGGGG | 17 | 3751 |
| HSV1-UL30-814 | − | GGGGGGGGAGGACGAGC | 17 | 3752 |
| HSV1-UL30-815 | − | GGACGAGCTGGCCTTTC | 17 | 3753 |
| HSV1-UL30-816 | − | CGAGCTGGCCTTTCCGG | 17 | 3754 |
| HSV1-UL30-817 | − | CTGGCCTTTCCGGTGGC | 17 | 3755 |
| HSV1-UL30-818 | − | TGGCCTTTCCGGTGGCC | 17 | 3756 |
| HSV1-UL30-819 | − | TCCGGTGGCCGGGCACC | 17 | 3757 |
| HSV1-UL30-820 | − | GGTGGCCGGGCACCCGG | 17 | 3758 |
| HSV1-UL30-821 | − | CGGGCACCCGGAGGACC | 17 | 3759 |
| HSV1-UL30-822 | − | CCTGTCCACCACCGCCC | 17 | 3760 |
| HSV1-UL30-823 | − | GTCCTCCTGTTTTCGCT | 17 | 3761 |
| HSV1-UL30-824 | − | ATCCCACCTGAACGAGC | 17 | 3762 |
| HSV1-UL30-825 | − | CCACCTGAACGAGCTGG | 17 | 3763 |
| HSV1-UL30-826 | − | TGAACGAGCTGGCGGCC | 17 | 3764 |
| HSV1-UL30-827 | − | GAACGAGCTGGCGGCCA | 17 | 3765 |
| HSV1-UL30-828 | − | AACGAGCTGGCGGCCAG | 17 | 3766 |
| HSV1-UL30-829 | − | GGGCCTGCCCACGCCCG | 17 | 3767 |
| HSV1-UL30-830 | − | GCCCACGCCCGTGGTTC | 17 | 3768 |
| HSV1-UL30-831 | − | CGAATTCGAGATGCTGT | 17 | 3769 |
| HSV1-UL30-832 | − | ACCCTTGTGAAACAGTA | 17 | 3770 |
| HSV1-UL30-833 | − | GGCCCCGAGTTCGTGAC | 17 | 3771 |
| HSV1-UL30-834 | − | GCCCCGAGTTCGTGACC | 17 | 3772 |
| HSV1-UL30-835 | − | ACATCATCAACTTCGAC | 17 | 3773 |
| HSV1-UL30-836 | − | CGACTGGCCCTTCTTGC | 17 | 3774 |
| HSV1-UL30-837 | − | CTTGCTGGCCAAGCTGA | 17 | 3775 |
| HSV1-UL30-838 | − | GCTGACGGACATTTACA | 17 | 3776 |
| HSV1-UL30-839 | − | CATTTACAAGGTCCCCC | 17 | 3777 |
| HSV1-UL30-840 | − | TACAAGGTCCCCCTGGA | 17 | 3778 |
| HSV1-UL30-841 | − | ACAAGGTCCCCCTGGAC | 17 | 3779 |
| HSV1-UL30-842 | − | GTCCCCCTGGACGGGTA | 17 | 3780 |
| HSV1-UL30-843 | − | GGGTACGGCCGCATGAA | 17 | 3781 |
| HSV1-UL30-844 | − | ACGGCCGCATGAACGGC | 17 | 3782 |
| HSV1-UL30-845 | − | CGGCCGCATGAACGGCC | 17 | 3783 |
| HSV1-UL30-846 | − | GGCCGCATGAACGGCCG | 17 | 3784 |
| HSV1-UL30-847 | − | GGGGCGTGTTTCGCGTG | 17 | 3785 |
| HSV1-UL30-848 | − | GGGCGTGTTTCGCGTGT | 17 | 3786 |
| HSV1-UL30-849 | − | TTTCGCGTGTGGGACAT | 17 | 3787 |
| HSV1-UL30-850 | − | GAAGCGCAGCAAGATAA | 17 | 3788 |
| HSV1-UL30-851 | − | AGCAAGATAAAGGTGAA | 17 | 3789 |
| HSV1-UL30-852 | − | GATAAAGGTGAACGGCA | 17 | 3790 |
| HSV1-UL30-853 | − | GTGAACATCGACATGTA | 17 | 3791 |
| HSV1-UL30-854 | − | TGAACATCGACATGTAC | 17 | 3792 |
| HSV1-UL30-855 | − | CTACAAGCTCAACGCCG | 17 | 3793 |
| HSV1-UL30-856 | − | GGCCGAAGCCGTCCTGA | 17 | 3794 |
| HSV1-UL30-857 | − | CCTGAAGGACAAGAAGA | 17 | 3795 |
| HSV1-UL30-858 | − | CCCGCCTACTACGCCGC | 17 | 3796 |
| HSV1-UL30-859 | − | CCGCCTACTACGCCGCC | 17 | 3797 |
| HSV1-UL30-860 | − | GCCGGGCCCGCGCAACG | 17 | 3798 |
| HSV1-UL30-861 | − | CCGGGCCCGCGCAACGC | 17 | 3799 |
| HSV1-UL30-862 | − | CGGGCCCGCGCAACGCG | 17 | 3800 |
| HSV1-UL30-863 | − | GCGCAACGCGGGGTGAT | 17 | 3801 |
| HSV1-UL30-864 | − | CGGCGAGTACTGCATAC | 17 | 3802 |
| HSV1-UL30-865 | − | CATACAGGATTCCCTGC | 17 | 3803 |
| HSV1-UL30-866 | − | ACAGGATTCCCTGCTGG | 17 | 3804 |
| HSV1-UL30-867 | − | CAGGATTCCCTGCTGGT | 17 | 3805 |
| HSV1-UL30-868 | − | TAAGTTTTTGCCCCATC | 17 | 3806 |
| HSV1-UL30-869 | − | GCCCCATCTGGAGCTCT | 17 | 3807 |
| HSV1-UL30-870 | − | CTCGGCCGTCGCGCGCT | 17 | 3808 |
| HSV1-UL30-871 | − | GGCCGTCGCGCGCTTGG | 17 | 3809 |
| HSV1-UL30-872 | − | GCCGTCGCGCGCTTGGC | 17 | 3810 |
| HSV1-UL30-873 | − | ACCCGCACCATCTACGA | 17 | 3811 |
| HSV1-UL30-874 | − | TACGTGCCTGCTGCGCC | 17 | 3812 |
| HSV1-UL30-875 | − | GCGCCTGGCCGACCAGA | 17 | 3813 |
| HSV1-UL30-876 | − | CGCCTGGCCGACCAGAA | 17 | 3814 |
| HSV1-UL30-877 | − | GAAGGGCTTTATTCTGC | 17 | 3815 |
| HSV1-UL30-878 | − | TATTCTGCCGGACACCC | 17 | 3816 |
| HSV1-UL30-879 | − | ATTCTGCCGGACACCCA | 17 | 3817 |
| HSV1-UL30-880 | − | TTCTGCCGGACACCCAG | 17 | 3818 |
| HSV1-UL30-881 | − | ACACCCAGGGGCGATTT | 17 | 3819 |
| HSV1-UL30-882 | − | CACCCAGGGGCGATTTA | 17 | 3820 |
| HSV1-UL30-883 | − | ACCCAGGGGCGATTTAG | 17 | 3821 |
| HSV1-UL30-884 | − | GGGCGATTTAGGGGCGC | 17 | 3822 |
| HSV1-UL30-885 | − | GGCGATTTAGGGGCGCC | 17 | 3823 |
| HSV1-UL30-886 | − | GCGATTTAGGGGCGCCG | 17 | 3824 |
| HSV1-UL30-887 | − | CGATTTAGGGGCGCCGG | 17 | 3825 |
| HSV1-UL30-888 | − | GATTTAGGGGCGCCGGG | 17 | 3826 |
| HSV1-UL30-889 | − | ATTTAGGGGCGCCGGGG | 17 | 3827 |
| HSV1-UL30-890 | − | TAGGGGCGCCGGGGGGG | 17 | 3828 |
| HSV1-UL30-891 | − | GGAGGCGCCCAAGCGTC | 17 | 3829 |
| HSV1-UL30-892 | − | AGCGTCCGGCCGCAGCC | 17 | 3830 |
| HSV1-UL30-893 | − | GCGTCCGGCCGCAGCCC | 17 | 3831 |
| HSV1-UL30-894 | − | TCCGGCCGCAGCCCGGG | 17 | 3832 |
| HSV1-UL30-895 | − | CGCAGCCCGGGAGGACG | 17 | 3833 |
| HSV1-UL30-896 | − | CCCGGGAGGACGAGGAG | 17 | 3834 |
| HSV1-UL30-897 | − | GGACGAGGAGCGGCCAG | 17 | 3835 |
| HSV1-UL30-898 | − | CGAGGAGCGGCCAGAGG | 17 | 3836 |
| HSV1-UL30-899 | − | GGAGCGGCCAGAGGAGG | 17 | 3837 |
| HSV1-UL30-900 | − | GAGCGGCCAGAGGAGGA | 17 | 3838 |
| HSV1-UL30-901 | − | AGCGGCCAGAGGAGGAG | 17 | 3839 |
| HSV1-UL30-902 | − | GCGGCCAGAGGAGGAGG | 17 | 3840 |
| HSV1-UL30-903 | − | GCCAGAGGAGGAGGGGG | 17 | 3841 |
| HSV1-UL30-904 | − | GGAGGAGGGGGAGGACG | 17 | 3842 |
| HSV1-UL30-905 | − | GGACGAGGACGAACGCG | 17 | 3843 |
| HSV1-UL30-906 | − | CGAGGACGAACGCGAGG | 17 | 3844 |
| HSV1-UL30-907 | − | GAGGACGAACGCGAGGA | 17 | 3845 |
| HSV1-UL30-908 | − | GACGAACGCGAGGAGGG | 17 | 3846 |
| HSV1-UL30-909 | − | ACGAACGCGAGGAGGGC | 17 | 3847 |
| HSV1-UL30-910 | − | CGAACGCGAGGAGGGCG | 17 | 3848 |
| HSV1-UL30-911 | − | GAACGCGAGGAGGGCGG | 17 | 3849 |
| HSV1-UL30-912 | − | AGGAGGGCGGGGGCGAG | 17 | 3850 |
| HSV1-UL30-913 | − | GGAGGGCGGGGGCGAGC | 17 | 3851 |
| HSV1-UL30-914 | − | CGGGGGCGAGCGGGAGC | 17 | 3852 |
| HSV1-UL30-915 | − | GGGCGAGCGGGAGCCGG | 17 | 3853 |
| HSV1-UL30-916 | − | GGCGAGCGGGAGCCGGA | 17 | 3854 |
| HSV1-UL30-917 | − | GGGAGCCGGAGGGCGCG | 17 | 3855 |
| HSV1-UL30-918 | − | GGAGCCGGAGGGCGCGC | 17 | 3856 |
| HSV1-UL30-919 | − | GGCGCGCGGGAGACCGC | 17 | 3857 |
| HSV1-UL30-920 | − | CGCGGGAGACCGCCGGC | 17 | 3858 |
| HSV1-UL30-921 | − | GACCGCCGGCAGGCACG | 17 | 3859 |
| HSV1-UL30-922 | − | ACCGCCGGCAGGCACGT | 17 | 3860 |
| HSV1-UL30-923 | − | CCGCCGGCAGGCACGTG | 17 | 3861 |
| HSV1-UL30-924 | − | CAGGCACGTGGGGTACC | 17 | 3862 |
| HSV1-UL30-925 | − | AGGCACGTGGGGTACCA | 17 | 3863 |
| HSV1-UL30-926 | − | GGCACGTGGGGTACCAG | 17 | 3864 |
| HSV1-UL30-927 | − | GCACGTGGGGTACCAGG | 17 | 3865 |
| HSV1-UL30-928 | − | TGGGGTACCAGGGGGCC | 17 | 3866 |
| HSV1-UL30-929 | − | GGGGTACCAGGGGGCCA | 17 | 3867 |
| HSV1-UL30-930 | − | GTCCTTGACCCCACTTC | 17 | 3868 |
| HSV1-UL30-931 | − | TCCTTGACCCCACTTCC | 17 | 3869 |
| HSV1-UL30-932 | − | GTTTCACGTGAACCCCG | 17 | 3870 |
| HSV1-UL30-933 | − | TCACGTGAACCCCGTGG | 17 | 3871 |
| HSV1-UL30-934 | − | GTACCCCAGCATCATCC | 17 | 3872 |
| HSV1-UL30-935 | − | TCAGCACGCTCTCCCTG | 17 | 3873 |
| HSV1-UL30-936 | − | CAGCACGCTCTCCCTGA | 17 | 3874 |
| HSV1-UL30-937 | − | CCTGAGGGCCGACGCAG | 17 | 3875 |
| HSV1-UL30-938 | − | CGACGCAGTGGCGCACC | 17 | 3876 |
| HSV1-UL30-939 | − | CGCAGTGGCGCACCTGG | 17 | 3877 |
| HSV1-UL30-940 | − | AGTGGCGCACCTGGAGG | 17 | 3878 |
| HSV1-UL30-941 | − | GTGGCGCACCTGGAGGC | 17 | 3879 |
| HSV1-UL30-942 | − | GCACCTGGAGGCGGGCA | 17 | 3880 |
| HSV1-UL30-943 | − | GGCGGGCAAGGACTACC | 17 | 3881 |
| HSV1-UL30-944 | − | GGACTACCTGGAGATCG | 17 | 3882 |
| HSV1-UL30-945 | − | CTACCTGGAGATCGAGG | 17 | 3883 |
| HSV1-UL30-946 | − | TACCTGGAGATCGAGGT | 17 | 3884 |
| HSV1-UL30-947 | − | ACCTGGAGATCGAGGTG | 17 | 3885 |
| HSV1-UL30-948 | − | CCTGGAGATCGAGGTGG | 17 | 3886 |
| HSV1-UL30-949 | − | CTGGAGATCGAGGTGGG | 17 | 3887 |
| HSV1-UL30-950 | − | TGGAGATCGAGGTGGGG | 17 | 3888 |
| HSV1-UL30-951 | − | TCGAGGTGGGGGGGCGA | 17 | 3889 |
| HSV1-UL30-952 | − | ACGGCTGTTCTTCGTCA | 17 | 3890 |
| HSV1-UL30-953 | − | TCCTCAGCATCCTCCTG | 17 | 3891 |
| HSV1-UL30-954 | − | CCTCAGCATCCTCCTGC | 17 | 3892 |
| HSV1-UL30-955 | − | GCATCCTCCTGCGGGAC | 17 | 3893 |
| HSV1-UL30-956 | − | GAAAGCAGATCCGCTCG | 17 | 3894 |
| HSV1-UL30-957 | − | TCCCCAGAGCAGCCCCG | 17 | 3895 |
| HSV1-UL30-958 | − | CCAGAGCAGCCCCGAGG | 17 | 3896 |
| HSV1-UL30-959 | − | CGAGGAGGCCGTGCTCC | 17 | 3897 |
| HSV1-UL30-960 | − | GCTCCTGGACAAGCAGC | 17 | 3898 |
| HSV1-UL30-961 | − | GCAGCAGGCCGCCATCA | 17 | 3899 |
| HSV1-UL30-962 | − | CAAGGTCGTGTGTAACT | 17 | 3900 |
| HSV1-UL30-963 | − | GTGTGTAACTCGGTGTA | 17 | 3901 |
| HSV1-UL30-964 | − | TGTGTAACTCGGTGTAC | 17 | 3902 |
| HSV1-UL30-965 | − | CTCGGTGTACGGGTTCA | 17 | 3903 |
| HSV1-UL30-966 | − | TCGGTGTACGGGTTCAC | 17 | 3904 |
| HSV1-UL30-967 | − | TTCACGGGAGTGCAGCA | 17 | 3905 |
| HSV1-UL30-968 | − | CCTGCACGTTGCCGCGA | 17 | 3906 |
| HSV1-UL30-969 | − | GCGACGGTGACGACCAT | 17 | 3907 |
| HSV1-UL30-970 | − | AGTACGTCCACGCGCGC | 17 | 3908 |
| HSV1-UL30-971 | − | GTACGTCCACGCGCGCT | 17 | 3909 |
| HSV1-UL30-972 | − | CGTCCACGCGCGCTGGG | 17 | 3910 |
| HSV1-UL30-973 | − | GGCCTTCGAACAGCTCC | 17 | 3911 |
| HSV1-UL30-974 | − | GCTCCTGGCCGATTTCC | 17 | 3912 |
| HSV1-UL30-975 | − | CCTGGCCGATTTCCCGG | 17 | 3913 |
| HSV1-UL30-976 | − | GGCCGATTTCCCGGAGG | 17 | 3914 |
| HSV1-UL30-977 | − | GCCGACATGCGCGCCCC | 17 | 3915 |
| HSV1-UL30-978 | − | CCGACATGCGCGCCCCC | 17 | 3916 |
| HSV1-UL30-979 | − | TCCATGCGCATCATCTA | 17 | 3917 |
| HSV1-UL30-980 | − | CCATGCGCATCATCTAC | 17 | 3918 |
| HSV1-UL30-981 | − | CATGCGCATCATCTACG | 17 | 3919 |
| HSV1-UL30-982 | − | CATCATCTACGGGGACA | 17 | 3920 |
| HSV1-UL30-983 | − | ATCTTTGTGCTGTGCCG | 17 | 3921 |
| HSV1-UL30-984 | − | GCTGTGCCGCGGCCTCA | 17 | 3922 |
| HSV1-UL30-985 | − | CGCGGCCTCACGGCCGC | 17 | 3923 |
| HSV1-UL30-986 | − | GCGGCCTCACGGCCGCC | 17 | 3924 |
| HSV1-UL30-987 | − | CACGGCCGCCGGGCTGA | 17 | 3925 |
| HSV1-UL30-988 | − | CGCCGGGCTGACGGCCG | 17 | 3926 |
| HSV1-UL30-989 | − | GCCGGGCTGACGGCCGT | 17 | 3927 |
| HSV1-UL30-990 | − | GGCCGTGGGCGACAAGA | 17 | 3928 |
| HSV1-UL30-991 | − | GCCAAGAAAAAGTACAT | 17 | 3929 |
| HSV1-UL30-992 | − | TACATCGGCGTCATCTA | 17 | 3930 |
| HSV1-UL30-993 | − | ACATCGGCGTCATCTAC | 17 | 3931 |
| HSV1-UL30-994 | − | CATCGGCGTCATCTACG | 17 | 3932 |
| HSV1-UL30-995 | − | ATCGGCGTCATCTACGG | 17 | 3933 |
| HSV1-UL30-996 | − | GGGTAAGATGCTCATCA | 17 | 3934 |
| HSV1-UL30-997 | − | GGTAAGATGCTCATCAA | 17 | 3935 |
| HSV1-UL30-998 | − | GATGCTCATCAAGGGCG | 17 | 3936 |
| HSV1-UL30-999 | − | CATCAAGGGCGTGGATC | 17 | 3937 |
| HSV1-UL30-1000 | − | TTATCAACCGCACCTCC | 17 | 3938 |
| HSV1-UL30-1001 | − | TATCAACCGCACCTCCA | 17 | 3939 |
| HSV1-UL30-1002 | − | CCGCACCTCCAGGGCCC | 17 | 3940 |
| HSV1-UL30-1003 | − | TACGACGATACCGTCTC | 17 | 3941 |
| HSV1-UL30-1004 | − | CGATACCGTCTCCGGAG | 17 | 3942 |
| HSV1-UL30-1005 | − | GTTAGCCGAGCGCCCCG | 17 | 3943 |
| HSV1-UL30-1006 | − | AGCCGAGCGCCCCGCGG | 17 | 3944 |
| HSV1-UL30-1007 | − | AGCGCCCCGCGGAGGAG | 17 | 3945 |
| HSV1-UL30-1008 | − | CCCCGCGGAGGAGTGGC | 17 | 3946 |
| HSV1-UL30-1009 | − | GGCGCGACCCCTGCCCG | 17 | 3947 |
| HSV1-UL30-1010 | − | GCGCGACCCCTGCCCGA | 17 | 3948 |
| HSV1-UL30-1011 | − | CCTGCCCGAGGGACTGC | 17 | 3949 |
| HSV1-UL30-1012 | − | GAGGGACTGCAGGCGTT | 17 | 3950 |
| HSV1-UL30-1013 | − | AGGGACTGCAGGCGTTC | 17 | 3951 |
| HSV1-UL30-1014 | − | GGGACTGCAGGCGTTCG | 17 | 3952 |
| HSV1-UL30-1015 | − | TCCTCGTAGACGCCCAT | 17 | 3953 |
| HSV1-UL30-1016 | − | TCGGCGCATCACCGACC | 17 | 3954 |
| HSV1-UL30-1017 | − | GCATCACCGACCCGGAG | 17 | 3955 |
| HSV1-UL30-1018 | − | CATCACCGACCCGGAGA | 17 | 3956 |
| HSV1-UL30-1019 | − | CCCGGAGAGGGACATCC | 17 | 3957 |
| HSV1-UL30-1020 | − | GTACACCAACAAGCGCC | 17 | 3958 |
| HSV1-UL30-1021 | − | GCGCCTGGCCCACCTGA | 17 | 3959 |
| HSV1-UL30-1022 | − | GGTGTATTACAAGCTCA | 17 | 3960 |
| HSV1-UL30-1023 | − | CATGGCCCGCCGCGCGC | 17 | 3961 |
| HSV1-UL30-1024 | − | GCAGGTCCCGTCCATCA | 17 | 3962 |
| HSV1-UL30-1025 | − | TCCCGTCCATCAAGGAC | 17 | 3963 |
| HSV1-UL30-1026 | − | GATCCCGTACGTGATCG | 17 | 3964 |
| HSV1-UL30-1027 | − | CGTGGCCCAGACCCGCG | 17 | 3965 |
| HSV1-UL30-1028 | − | CCAGACCCGCGAGGTAG | 17 | 3966 |
| HSV1-UL30-1029 | − | CCGCGAGGTAGAGGAGA | 17 | 3967 |
| HSV1-UL30-1030 | − | TAGAGGAGACGGTCGCG | 17 | 3968 |
| HSV1-UL30-1031 | − | GGAGACGGTCGCGCGGC | 17 | 3969 |
| HSV1-UL30-1032 | − | CTAGACGCCGCCGCCCC | 17 | 3970 |
| HSV1-UL30-1033 | − | TAGACGCCGCCGCCCCA | 17 | 3971 |
| HSV1-UL30-1034 | − | AGACGCCGCCGCCCCAG | 17 | 3972 |
| HSV1-UL30-1035 | − | CGAGCCCGCCCCCCCCG | 17 | 3973 |
| HSV1-UL30-1036 | − | CGCGGCCCTGCCCTCCC | 17 | 3974 |
| HSV1-UL30-1037 | − | CCCCGGCCAAGCGCCCC | 17 | 3975 |
| HSV1-UL30-1038 | − | CCCGGCCAAGCGCCCCC | 17 | 3976 |
| HSV1-UL30-1039 | − | GTCGCCTGCCGACCCCC | 17 | 3977 |
| HSV1-UL30-1040 | − | TCGCCTGCCGACCCCCC | 17 | 3978 |
| HSV1-UL30-1041 | − | CCTGCCGACCCCCCGGG | 17 | 3979 |
| HSV1-UL30-1042 | − | CAAGCCCCGCAAGCTGC | 17 | 3980 |
| HSV1-UL30-1043 | − | GCTGCTGGTGTCCGAGC | 17 | 3981 |
| HSV1-UL30-1044 | − | GGTGTCCGAGCTGGCCG | 17 | 3982 |
| HSV1-UL30-1045 | − | GCATACGCCATTGCCCA | 17 | 3983 |
| HSV1-UL30-1046 | − | CGGCGTCGCCCTGAACA | 17 | 3984 |
| HSV1-UL30-1047 | − | TTACTTCTCCCACCTGT | 17 | 3985 |
| HSV1-UL30-1048 | − | TACTTCTCCCACCTGTT | 17 | 3986 |
| HSV1-UL30-1049 | − | ACTTCTCCCACCTGTTG | 17 | 3987 |
| HSV1-UL30-1050 | − | CTTCTCCCACCTGTTGG | 17 | 3988 |
| HSV1-UL30-1051 | − | CTCCCACCTGTTGGGGG | 17 | 3989 |
| HSV1-UL30-1052 | − | GGCGTGCGTGACATTCA | 17 | 3990 |
| HSV1-UL30-1053 | − | ACATTCAAGGCCCTGTT | 17 | 3991 |
| HSV1-UL30-1054 | − | CATTCAAGGCCCTGTTT | 17 | 3992 |
| HSV1-UL30-1055 | − | CCGAGAGTCTGTTAAAA | 17 | 3993 |
| HSV1-UL30-1056 | − | GGTTTATTCCCGAAGTG | 17 | 3994 |
| HSV1-UL30-1057 | − | CGAAGTGTGGCACCCCC | 17 | 3995 |
| HSV1-UL30-1058 | − | GCACCCCCCGGACGACG | 17 | 3996 |
| HSV1-UL30-1059 | − | CGGACGACGTGGCCGCG | 17 | 3997 |
| HSV1-UL30-1060 | − | ACGTGGCCGCGCGGCTC | 17 | 3998 |
| HSV1-UL30-1061 | − | GCGCGGCTCCGGACCGC | 17 | 3999 |
| HSV1-UL30-1062 | − | CGCGGCTCCGGACCGCA | 17 | 4000 |
| HSV1-UL30-1063 | − | CTCCGGACCGCAGGGTT | 17 | 4001 |
| HSV1-UL30-1064 | − | TCCGGACCGCAGGGTTC | 17 | 4002 |
| HSV1-UL30-1065 | − | CCGGACCGCAGGGTTCG | 17 | 4003 |
| HSV1-UL30-1066 | − | GACCGCAGGGTTCGGGG | 17 | 4004 |
| HSV1-UL30-1067 | − | CGCAGGGTTCGGGGCGG | 17 | 4005 |
| HSV1-UL30-1068 | − | GCAGGGTTCGGGGCGGT | 17 | 4006 |
| HSV1-UL30-1069 | − | TTCGGGGCGGTGGGTGC | 17 | 4007 |
| HSV1-UL30-1070 | − | GGTGGGTGCCGGCGCTA | 17 | 4008 |
| HSV1-UL30-1071 | − | GGGTGCCGGCGCTACGG | 17 | 4009 |
| HSV1-UL30-1072 | − | TGCCGGCGCTACGGCGG | 17 | 4010 |
| HSV1-UL30-1073 | + | AGTTTCCTCCGCCGTAGCGC | 20 | 4011 |
| HSV1-UL30-1074 | + | ACCCACCGCCCCGAACCCTG | 20 | 4012 |
| HSV1-UL30-1075 | + | CCGCCCCGAACCCTGCGGTC | 20 | 4013 |
| HSV1-UL30-1076 | + | CCCTGCGGTCCGGAGCCGCG | 20 | 4014 |
| HSV1-UL30-1077 | + | AGCCGCGCGGCCACGTCGTC | 20 | 4015 |
| HSV1-UL30-1078 | + | GCCGCGCGGCCACGTCGTCC | 20 | 4016 |
| HSV1-UL30-1079 | + | CCGCGCGGCCACGTCGTCCG | 20 | 4017 |
| HSV1-UL30-1080 | + | CGCGCGGCCACGTCGTCCGG | 20 | 4018 |
| HSV1-UL30-1081 | + | GCGCGGCCACGTCGTCCGGG | 20 | 4019 |
| HSV1-UL30-1082 | + | GTCCGGGGGGTGCCACACTT | 20 | 4020 |
| HSV1-UL30-1083 | + | TCCGGGGGGTGCCACACTTC | 20 | 4021 |
| HSV1-UL30-1084 | + | AAACCTTTTTAACAGACTCT | 20 | 4022 |
| HSV1-UL30-1085 | + | TAACAGACTCTCGGTGATCT | 20 | 4023 |
| HSV1-UL30-1086 | + | TCTTGGCGTTATTCCCAAAC | 20 | 4024 |
| HSV1-UL30-1087 | + | CTTGGCGTTATTCCCAAACA | 20 | 4025 |
| HSV1-UL30-1088 | + | TCACGCACGCCGCCCCCAAC | 20 | 4026 |
| HSV1-UL30-1089 | + | CGCACGCCGCCCCCAACAGG | 20 | 4027 |
| HSV1-UL30-1090 | + | GCACGCCGCCCCCAACAGGT | 20 | 4028 |
| HSV1-UL30-1091 | + | AGAAGTAATAGTCCGTGTTC | 20 | 4029 |
| HSV1-UL30-1092 | + | GAAGTAATAGTCCGTGTTCA | 20 | 4030 |
| HSV1-UL30-1093 | + | CCGTGTTCAGGGCGACGCCG | 20 | 4031 |
| HSV1-UL30-1094 | + | CGTGTTCAGGGCGACGCCGT | 20 | 4032 |
| HSV1-UL30-1095 | + | CAGGGCGACGCCGTGGGCAA | 20 | 4033 |
| HSV1-UL30-1096 | + | GCCGTGGGCAATGGCGTATG | 20 | 4034 |
| HSV1-UL30-1097 | + | CCGTGGGCAATGGCGTATGC | 20 | 4035 |
| HSV1-UL30-1098 | + | AATGGCGTATGCGGGATCCT | 20 | 4036 |
| HSV1-UL30-1099 | + | TGCGGGATCCTCGGCCAGCT | 20 | 4037 |
| HSV1-UL30-1100 | + | GCTCGGACACCAGCAGCTTG | 20 | 4038 |
| HSV1-UL30-1101 | + | CTCGGACACCAGCAGCTTGC | 20 | 4039 |
| HSV1-UL30-1102 | + | TCGGACACCAGCAGCTTGCG | 20 | 4040 |
| HSV1-UL30-1103 | + | CACCAGCAGCTTGCGGGGCT | 20 | 4041 |
| HSV1-UL30-1104 | + | CGGGGCTTGGACGCGCCTCC | 20 | 4042 |
| HSV1-UL30-1105 | + | GGGGCTTGGACGCGCCTCCC | 20 | 4043 |
| HSV1-UL30-1106 | + | GGGCTTGGACGCGCCTCCCG | 20 | 4044 |
| HSV1-UL30-1107 | + | GGCTTGGACGCGCCTCCCGG | 20 | 4045 |
| HSV1-UL30-1108 | + | GCTTGGACGCGCCTCCCGGG | 20 | 4046 |
| HSV1-UL30-1109 | + | GGACGCGCCTCCCGGGGGGT | 20 | 4047 |
| HSV1-UL30-1110 | + | GCGCCTCCCGGGGGGTCGGC | 20 | 4048 |
| HSV1-UL30-1111 | + | CCCGGGGGGTCGGCAGGCGA | 20 | 4049 |
| HSV1-UL30-1112 | + | CGGCAGGCGACGGCGTCTCC | 20 | 4050 |
| HSV1-UL30-1113 | + | GGCAGGCGACGGCGTCTCCC | 20 | 4051 |
| HSV1-UL30-1114 | + | GCAGGCGACGGCGTCTCCCG | 20 | 4052 |
| HSV1-UL30-1115 | + | CAGGCGACGGCGTCTCCCGG | 20 | 4053 |
| HSV1-UL30-1116 | + | CGGCGTCTCCCGGGGGCGCT | 20 | 4054 |
| HSV1-UL30-1117 | + | GTCTCCCGGGGGCGCTTGGC | 20 | 4055 |
| HSV1-UL30-1118 | + | TCTCCCGGGGGCGCTTGGCC | 20 | 4056 |
| HSV1-UL30-1119 | + | CTCCCGGGGGCGCTTGGCCG | 20 | 4057 |
| HSV1-UL30-1120 | + | CCGGGGGCGCTTGGCCGGGG | 20 | 4058 |
| HSV1-UL30-1121 | + | CGGGGGCGCTTGGCCGGGGA | 20 | 4059 |
| HSV1-UL30-1122 | + | GGCGCTTGGCCGGGGAGGGC | 20 | 4060 |
| HSV1-UL30-1123 | + | GCGCTTGGCCGGGGAGGGCA | 20 | 4061 |
| HSV1-UL30-1124 | + | GGCCGGGGAGGGCAGGGCCG | 20 | 4062 |
| HSV1-UL30-1125 | + | GCCGGGGAGGGCAGGGCCGC | 20 | 4063 |
| HSV1-UL30-1126 | + | CCGGGGAGGGCAGGGCCGCG | 20 | 4064 |
| HSV1-UL30-1127 | + | CGGGGAGGGCAGGGCCGCGG | 20 | 4065 |
| HSV1-UL30-1128 | + | GGGGAGGGCAGGGCCGCGGG | 20 | 4066 |
| HSV1-UL30-1129 | + | GGGAGGGCAGGGCCGCGGGG | 20 | 4067 |
| HSV1-UL30-1130 | + | GGAGGGCAGGGCCGCGGGGG | 20 | 4068 |
| HSV1-UL30-1131 | + | GGGCAGGGCCGCGGGGGGGG | 20 | 4069 |
| HSV1-UL30-1132 | + | GGCAGGGCCGCGGGGGGGGC | 20 | 4070 |
| HSV1-UL30-1133 | + | GGGGGGGCGGGCTCGTCCCC | 20 | 4071 |
| HSV1-UL30-1134 | + | GGGGGGCGGGCTCGTCCCCT | 20 | 4072 |
| HSV1-UL30-1135 | + | GGGGGCGGGCTCGTCCCCTG | 20 | 4073 |
| HSV1-UL30-1136 | + | GGCGGGCTCGTCCCCTGGGG | 20 | 4074 |
| HSV1-UL30-1137 | + | GGGCTCGTCCCCTGGGGCGG | 20 | 4075 |
| HSV1-UL30-1138 | + | GGGCGGCGGCGTCTAGCTCG | 20 | 4076 |
| HSV1-UL30-1139 | + | CGGCGGCGTCTAGCTCGCGG | 20 | 4077 |
| HSV1-UL30-1140 | + | GGCGGCGTCTAGCTCGCGGA | 20 | 4078 |
| HSV1-UL30-1141 | + | GGCGTCTAGCTCGCGGAGGG | 20 | 4079 |
| HSV1-UL30-1142 | + | CGACCGTCTCCTCTACCTCG | 20 | 4080 |
| HSV1-UL30-1143 | + | GACCGTCTCCTCTACCTCGC | 20 | 4081 |
| HSV1-UL30-1144 | + | TCTCCTCTACCTCGCGGGTC | 20 | 4082 |
| HSV1-UL30-1145 | + | CTCCTCTACCTCGCGGGTCT | 20 | 4083 |
| HSV1-UL30-1146 | + | GTCTGGGCCACGATCACGTA | 20 | 4084 |
| HSV1-UL30-1147 | + | TCTGGGCCACGATCACGTAC | 20 | 4085 |
| HSV1-UL30-1148 | + | CCACGATCACGTACGGGATC | 20 | 4086 |
| HSV1-UL30-1149 | + | GTACGGGATCCGGTCCTTGA | 20 | 4087 |
| HSV1-UL30-1150 | + | GGGATCCGGTCCTTGATGGA | 20 | 4088 |
| HSV1-UL30-1151 | + | GGATCCGGTCCTTGATGGAC | 20 | 4089 |
| HSV1-UL30-1152 | + | TGATGGACGGGACCTGCGCG | 20 | 4090 |
| HSV1-UL30-1153 | + | TGGACGGGACCTGCGCGCGG | 20 | 4091 |
| HSV1-UL30-1154 | + | GGACGGGACCTGCGCGCGGC | 20 | 4092 |
| HSV1-UL30-1155 | + | TGAGCTTGTAATACACCGTC | 20 | 4093 |
| HSV1-UL30-1156 | + | GCTTGTAATACACCGTCAGG | 20 | 4094 |
| HSV1-UL30-1157 | + | CTTGTAATACACCGTCAGGT | 20 | 4095 |
| HSV1-UL30-1158 | + | AATACACCGTCAGGTGGGCC | 20 | 4096 |
| HSV1-UL30-1159 | + | CAGGTGGGCCAGGCGCTTGT | 20 | 4097 |
| HSV1-UL30-1160 | + | CGCTTGTTGGTGTACGCGCG | 20 | 4098 |
| HSV1-UL30-1161 | + | GCTTGTTGGTGTACGCGCGC | 20 | 4099 |
| HSV1-UL30-1162 | + | GCGCGGGTGTCTGCTCAGTT | 20 | 4100 |
| HSV1-UL30-1163 | + | CGGGTGTCTGCTCAGTTCGG | 20 | 4101 |
| HSV1-UL30-1164 | + | GTCTGCTCAGTTCGGCGGTG | 20 | 4102 |
| HSV1-UL30-1165 | + | CGGCGGTGAGGACAAAGTCC | 20 | 4103 |
| HSV1-UL30-1166 | + | AAGTCCTGGATGTCCCTCTC | 20 | 4104 |
| HSV1-UL30-1167 | + | AGTCCTGGATGTCCCTCTCC | 20 | 4105 |
| HSV1-UL30-1168 | + | CTGGATGTCCCTCTCCGGGT | 20 | 4106 |
| HSV1-UL30-1169 | + | CCGGGTCGGTGATGCGCCGA | 20 | 4107 |
| HSV1-UL30-1170 | + | CGGGTCGGTGATGCGCCGAT | 20 | 4108 |
| HSV1-UL30-1171 | + | TGCGCCGATGGGCGTCTACG | 20 | 4109 |
| HSV1-UL30-1172 | + | CCGATGGGCGTCTACGAGGA | 20 | 4110 |
| HSV1-UL30-1173 | + | CCCGAACGCCTGCAGTCCCT | 20 | 4111 |
| HSV1-UL30-1174 | + | CCGAACGCCTGCAGTCCCTC | 20 | 4112 |
| HSV1-UL30-1175 | + | ACGCCTGCAGTCCCTCGGGC | 20 | 4113 |
| HSV1-UL30-1176 | + | CGCCTGCAGTCCCTCGGGCA | 20 | 4114 |
| HSV1-UL30-1177 | + | GCCTGCAGTCCCTCGGGCAG | 20 | 4115 |
| HSV1-UL30-1178 | + | TCGCGCCAGCCACTCCTCCG | 20 | 4116 |
| HSV1-UL30-1179 | + | CGCGCCAGCCACTCCTCCGC | 20 | 4117 |
| HSV1-UL30-1180 | + | GCGCCAGCCACTCCTCCGCG | 20 | 4118 |
| HSV1-UL30-1181 | + | CCACTCCTCCGCGGGGCGCT | 20 | 4119 |
| HSV1-UL30-1182 | + | CGCGGGGCGCTCGGCTAACG | 20 | 4120 |
| HSV1-UL30-1183 | + | GGGGCGCTCGGCTAACGCGG | 20 | 4121 |
| HSV1-UL30-1184 | + | GGCTAACGCGGCGGCCGCTC | 20 | 4122 |
| HSV1-UL30-1185 | + | CGCGGCGGCCGCTCCGGAGA | 20 | 4123 |
| HSV1-UL30-1186 | + | CGGTATCGTCGTAAAACAGC | 20 | 4124 |
| HSV1-UL30-1187 | + | CGTAAAACAGCAGGTCGACC | 20 | 4125 |
| HSV1-UL30-1188 | + | GTAAAACAGCAGGTCGACCA | 20 | 4126 |
| HSV1-UL30-1189 | + | CAGCAGGTCGACCAGGGCCC | 20 | 4127 |
| HSV1-UL30-1190 | + | CAGGTCGACCAGGGCCCTGG | 20 | 4128 |
| HSV1-UL30-1191 | + | CGACCAGGGCCCTGGAGGTG | 20 | 4129 |
| HSV1-UL30-1192 | + | GACGCCGATGTACTTTTTCT | 20 | 4130 |
| HSV1-UL30-1193 | + | CTTGGCGATCAGCAGCAGCT | 20 | 4131 |
| HSV1-UL30-1194 | + | CTTTTCGCACTCGAGTTTGA | 20 | 4132 |
| HSV1-UL30-1195 | + | TTTTCGCACTCGAGTTTGAT | 20 | 4133 |
| HSV1-UL30-1196 | + | TTTCGCACTCGAGTTTGATG | 20 | 4134 |
| HSV1-UL30-1197 | + | TTCGCACTCGAGTTTGATGG | 20 | 4135 |
| HSV1-UL30-1198 | + | TCGCACTCGAGTTTGATGGG | 20 | 4136 |
| HSV1-UL30-1199 | + | GAAACAGCGCGCGCGAGATG | 20 | 4137 |
| HSV1-UL30-1200 | + | GCTCGCCATCTTGTCGCCCA | 20 | 4138 |
| HSV1-UL30-1201 | + | GTCGCCCACGGCCGTCAGCC | 20 | 4139 |
| HSV1-UL30-1202 | + | GCCCACGGCCGTCAGCCCGG | 20 | 4140 |
| HSV1-UL30-1203 | + | CCGTCAGCCCGGCGGCCGTG | 20 | 4141 |
| HSV1-UL30-1204 | + | GCCCGGCGGCCGTGAGGCCG | 20 | 4142 |
| HSV1-UL30-1205 | + | GCCGCGGCACAGCACAAAGA | 20 | 4143 |
| HSV1-UL30-1206 | + | GTCCCCGTAGATGATGCGCA | 20 | 4144 |
| HSV1-UL30-1207 | + | GTAGATGATGCGCATGGAAT | 20 | 4145 |
| HSV1-UL30-1208 | + | TAGATGATGCGCATGGAATA | 20 | 4146 |
| HSV1-UL30-1209 | + | GATGCGCATGGAATAGGGCC | 20 | 4147 |
| HSV1-UL30-1210 | + | ATGCGCATGGAATAGGGCCC | 20 | 4148 |
| HSV1-UL30-1211 | + | TGCGCATGGAATAGGGCCCG | 20 | 4149 |
| HSV1-UL30-1212 | + | GCGCATGGAATAGGGCCCGG | 20 | 4150 |
| HSV1-UL30-1213 | + | GGGCCCGGGGGCGCGCATGT | 20 | 4151 |
| HSV1-UL30-1214 | + | GCGCGCATGTCGGCCGCCTC | 20 | 4152 |
| HSV1-UL30-1215 | + | CGCGCATGTCGGCCGCCTCC | 20 | 4153 |
| HSV1-UL30-1216 | + | GTCGGCCGCCTCCGGGAAAT | 20 | 4154 |
| HSV1-UL30-1217 | + | CCGCCTCCGGGAAATCGGCC | 20 | 4155 |
| HSV1-UL30-1218 | + | ATCGGCCAGGAGCTGTTCGA | 20 | 4156 |
| HSV1-UL30-1219 | + | CGAAGGCCGCCCAGCGCGCG | 20 | 4157 |
| HSV1-UL30-1220 | + | AGCGCGCGTGGACGTACTCG | 20 | 4158 |
| HSV1-UL30-1221 | + | GCGCGCGTGGACGTACTCGC | 20 | 4159 |
| HSV1-UL30-1222 | + | GGGTCGCGAGCAGCATCTCG | 20 | 4160 |
| HSV1-UL30-1223 | + | GAGCAGCATCTCGCGGCCGA | 20 | 4161 |
| HSV1-UL30-1224 | + | GCCGATGGTCGTCACCGTCG | 20 | 4162 |
| HSV1-UL30-1225 | + | TCACCGTCGCGGCAACGTGC | 20 | 4163 |
| HSV1-UL30-1226 | + | GTCGCGGCAACGTGCAGGCA | 20 | 4164 |
| HSV1-UL30-1227 | + | CGGCAACGTGCAGGCACGGC | 20 | 4165 |
| HSV1-UL30-1228 | + | CGAGTTACACACGACCTTGA | 20 | 4166 |
| HSV1-UL30-1229 | + | GTTACACACGACCTTGATGG | 20 | 4167 |
| HSV1-UL30-1230 | + | TGGCGGCCTGCTGCTTGTCC | 20 | 4168 |
| HSV1-UL30-1231 | + | CTGCTGCTTGTCCAGGAGCA | 20 | 4169 |
| HSV1-UL30-1232 | + | GTCCAGGAGCACGGCCTCCT | 20 | 4170 |
| HSV1-UL30-1233 | + | TCCAGGAGCACGGCCTCCTC | 20 | 4171 |
| HSV1-UL30-1234 | + | CCAGGAGCACGGCCTCCTCG | 20 | 4172 |
| HSV1-UL30-1235 | + | CGGCCTCCTCGGGGCTGCTC | 20 | 4173 |
| HSV1-UL30-1236 | + | GGCCTCCTCGGGGCTGCTCT | 20 | 4174 |
| HSV1-UL30-1237 | + | GCCTCCTCGGGGCTGCTCTG | 20 | 4175 |
| HSV1-UL30-1238 | + | TGCTCTGGGGAATCCGCGAG | 20 | 4176 |
| HSV1-UL30-1239 | + | CGAGCGGATCTGCTTTCGCA | 20 | 4177 |
| HSV1-UL30-1240 | + | GCATGGCGAGCCAGTCCCGC | 20 | 4178 |
| HSV1-UL30-1241 | + | TGGCGAGCCAGTCCCGCAGG | 20 | 4179 |
| HSV1-UL30-1242 | + | AGTCCCGCAGGAGGATGCTG | 20 | 4180 |
| HSV1-UL30-1243 | + | CCCGCAGGAGGATGCTGAGG | 20 | 4181 |
| HSV1-UL30-1244 | + | GCCCCCCCACCTCGATCTCC | 20 | 4182 |
| HSV1-UL30-1245 | + | GGTAGTCCTTGCCCGCCTCC | 20 | 4183 |
| HSV1-UL30-1246 | + | CTCCAGGTGCGCCACTGCGT | 20 | 4184 |
| HSV1-UL30-1247 | + | GCGCCACTGCGTCGGCCCTC | 20 | 4185 |
| HSV1-UL30-1248 | + | CGCCACTGCGTCGGCCCTCA | 20 | 4186 |
| HSV1-UL30-1249 | + | GGGAGAGCGTGCTGAAGCAC | 20 | 4187 |
| HSV1-UL30-1250 | + | GCGTGCTGAAGCACAGGTTG | 20 | 4188 |
| HSV1-UL30-1251 | + | CGTGCTGAAGCACAGGTTGT | 20 | 4189 |
| HSV1-UL30-1252 | + | TGAAGCACAGGTTGTGGGCC | 20 | 4190 |
| HSV1-UL30-1253 | + | GTTGTGGGCCTGGATGATGC | 20 | 4191 |
| HSV1-UL30-1254 | + | TTGTGGGCCTGGATGATGCT | 20 | 4192 |
| HSV1-UL30-1255 | + | TGTGGGCCTGGATGATGCTG | 20 | 4193 |
| HSV1-UL30-1256 | + | CCTGGATGATGCTGGGGTAC | 20 | 4194 |
| HSV1-UL30-1257 | + | GATGATGCTGGGGTACAGGC | 20 | 4195 |
| HSV1-UL30-1258 | + | GGCAAAGTCGAACACCACCA | 20 | 4196 |
| HSV1-UL30-1259 | + | GCAAAGTCGAACACCACCAC | 20 | 4197 |
| HSV1-UL30-1260 | + | CAAAGTCGAACACCACCACG | 20 | 4198 |
| HSV1-UL30-1261 | + | CACGGGGTTCACGTGAAACC | 20 | 4199 |
| HSV1-UL30-1262 | + | GTTCACGTGAAACCCGGAAG | 20 | 4200 |
| HSV1-UL30-1263 | + | TTCACGTGAAACCCGGAAGT | 20 | 4201 |
| HSV1-UL30-1264 | + | TCACGTGAAACCCGGAAGTG | 20 | 4202 |
| HSV1-UL30-1265 | + | GAAACCCGGAAGTGGGGTCA | 20 | 4203 |
| HSV1-UL30-1266 | + | GGAAGTGGGGTCAAGGACCC | 20 | 4204 |
| HSV1-UL30-1267 | + | GGTCAAGGACCCTGGCCCCC | 20 | 4205 |
| HSV1-UL30-1268 | + | CTGGTACCCCACGTGCCTGC | 20 | 4206 |
| HSV1-UL30-1269 | + | GTACCCCACGTGCCTGCCGG | 20 | 4207 |
| HSV1-UL30-1270 | + | GCGGTCTCCCGCGCGCCCTC | 20 | 4208 |
| HSV1-UL30-1271 | + | TCGTCCTCCCCCTCCTCCTC | 20 | 4209 |
| HSV1-UL30-1272 | + | CTGGCCGCTCCTCGTCCTCC | 20 | 4210 |
| HSV1-UL30-1273 | + | TGGCCGCTCCTCGTCCTCCC | 20 | 4211 |
| HSV1-UL30-1274 | + | CTCCTCGTCCTCCCGGGCTG | 20 | 4212 |
| HSV1-UL30-1275 | + | TCGTCCTCCCGGGCTGCGGC | 20 | 4213 |
| HSV1-UL30-1276 | + | CCGGGCTGCGGCCGGACGCT | 20 | 4214 |
| HSV1-UL30-1277 | + | CGGGCTGCGGCCGGACGCTT | 20 | 4215 |
| HSV1-UL30-1278 | + | ACGCTTGGGCGCCTCCCCCC | 20 | 4216 |
| HSV1-UL30-1279 | + | CGGCGCCCCTAAATCGCCCC | 20 | 4217 |
| HSV1-UL30-1280 | + | GGCGCCCCTAAATCGCCCCT | 20 | 4218 |
| HSV1-UL30-1281 | + | CTAAATCGCCCCTGGGTGTC | 20 | 4219 |
| HSV1-UL30-1282 | + | CCGGCAGAATAAAGCCCTTC | 20 | 4220 |
| HSV1-UL30-1283 | + | CAGAATAAAGCCCTTCTGGT | 20 | 4221 |
| HSV1-UL30-1284 | + | TAAAGCCCTTCTGGTCGGCC | 20 | 4222 |
| HSV1-UL30-1285 | + | TCTGGTCGGCCAGGCGCAGC | 20 | 4223 |
| HSV1-UL30-1286 | + | GCAGCAGGCACGTAAAGACG | 20 | 4224 |
| HSV1-UL30-1287 | + | ACGTAAAGACGCGGATCTGC | 20 | 4225 |
| HSV1-UL30-1288 | + | GATCTGCTGGCCGTCGTAGA | 20 | 4226 |
| HSV1-UL30-1289 | + | GCTGGCCGTCGTAGATGGTG | 20 | 4227 |
| HSV1-UL30-1290 | + | CTGGCCGTCGTAGATGGTGC | 20 | 4228 |
| HSV1-UL30-1291 | + | AATACCCGCCAAGCGCGCGA | 20 | 4229 |
| HSV1-UL30-1292 | + | CGACGGCCGAGAGCTCCAGA | 20 | 4230 |
| HSV1-UL30-1293 | + | GACGGCCGAGAGCTCCAGAT | 20 | 4231 |
| HSV1-UL30-1294 | + | ACGGCCGAGAGCTCCAGATG | 20 | 4232 |
| HSV1-UL30-1295 | + | GCAAAAACTTAAAAAACAGC | 20 | 4233 |
| HSV1-UL30-1296 | + | AAAACAGCTGGCCCACCAGC | 20 | 4234 |
| HSV1-UL30-1297 | + | AAACAGCTGGCCCACCAGCA | 20 | 4235 |
| HSV1-UL30-1298 | + | GCCGATCACCCCGCGTTGCG | 20 | 4236 |
| HSV1-UL30-1299 | + | CCGATCACCCCGCGTTGCGC | 20 | 4237 |
| HSV1-UL30-1300 | + | CACCCCGCGTTGCGCGGGCC | 20 | 4238 |
| HSV1-UL30-1301 | + | CCCGCGTTGCGCGGGCCCGG | 20 | 4239 |
| HSV1-UL30-1302 | + | CGCGGGCCCGGCGGCGTAGT | 20 | 4240 |
| HSV1-UL30-1303 | + | GGGCCCGGCGGCGTAGTAGG | 20 | 4241 |
| HSV1-UL30-1304 | + | GGCCCGGCGGCGTAGTAGGC | 20 | 4242 |
| HSV1-UL30-1305 | + | GCCCGGCGGCGTAGTAGGCG | 20 | 4243 |
| HSV1-UL30-1306 | + | CGGGGATGTCGCGATAGCTC | 20 | 4244 |
| HSV1-UL30-1307 | + | GGTCCTTCTTCTTGTCCTTC | 20 | 4245 |
| HSV1-UL30-1308 | + | CTTCTTCTTGTCCTTCAGGA | 20 | 4246 |
| HSV1-UL30-1309 | + | CTTGTCCTTCAGGACGGCTT | 20 | 4247 |
| HSV1-UL30-1310 | + | CTTCAGGACGGCTTCGGCCA | 20 | 4248 |
| HSV1-UL30-1311 | + | GCTCGAGAGCTTGATCTTGT | 20 | 4249 |
| HSV1-UL30-1312 | + | CCTTTATCTTGCTGCGCTTC | 20 | 4250 |
| HSV1-UL30-1313 | + | TCTTGCTGCGCTTCTGGAAG | 20 | 4251 |
| HSV1-UL30-1314 | + | TGCGCTTCTGGAAGTGGCTC | 20 | 4252 |
| HSV1-UL30-1315 | + | CCCACACGCGAAACACGCCC | 20 | 4253 |
| HSV1-UL30-1316 | + | ACACGCCCCGGCCGTTCATG | 20 | 4254 |
| HSV1-UL30-1317 | + | TCATGCGGCCGTACCCGTCC | 20 | 4255 |
| HSV1-UL30-1318 | + | CATGCGGCCGTACCCGTCCA | 20 | 4256 |
| HSV1-UL30-1319 | + | ATGCGGCCGTACCCGTCCAG | 20 | 4257 |
| HSV1-UL30-1320 | + | TGCGGCCGTACCCGTCCAGG | 20 | 4258 |
| HSV1-UL30-1321 | + | CTTGTAAATGTCCGTCAGCT | 20 | 4259 |
| HSV1-UL30-1322 | + | CGTCAGCTTGGCCAGCAAGA | 20 | 4260 |
| HSV1-UL30-1323 | + | GTCAGCTTGGCCAGCAAGAA | 20 | 4261 |
| HSV1-UL30-1324 | + | GAAGTTGATGATGTTGTACC | 20 | 4262 |
| HSV1-UL30-1325 | + | GTTGTACCCGGTCACGAACT | 20 | 4263 |
| HSV1-UL30-1326 | + | TTGTACCCGGTCACGAACTC | 20 | 4264 |
| HSV1-UL30-1327 | + | TGTACCCGGTCACGAACTCG | 20 | 4265 |
| HSV1-UL30-1328 | + | CGGGGCCGTACTGTTTCACA | 20 | 4266 |
| HSV1-UL30-1329 | + | GGGGCCGTACTGTTTCACAA | 20 | 4267 |
| HSV1-UL30-1330 | + | CTGTTTCACAAGGGTCATGA | 20 | 4268 |
| HSV1-UL30-1331 | + | GCTGTCGAATTCCAGAACCA | 20 | 4269 |
| HSV1-UL30-1332 | + | CTGTCGAATTCCAGAACCAC | 20 | 4270 |
| HSV1-UL30-1333 | + | GAATTCCAGAACCACGGGCG | 20 | 4271 |
| HSV1-UL30-1334 | + | AATTCCAGAACCACGGGCGT | 20 | 4272 |
| HSV1-UL30-1335 | + | CCAGAACCACGGGCGTGGGC | 20 | 4273 |
| HSV1-UL30-1336 | + | CACGGGCGTGGGCAGGCCCC | 20 | 4274 |
| HSV1-UL30-1337 | + | CCCTGGCCGCCAGCTCGTTC | 20 | 4275 |
| HSV1-UL30-1338 | + | TGGCCGCCAGCTCGTTCAGG | 20 | 4276 |
| HSV1-UL30-1339 | + | GGCCGCCAGCTCGTTCAGGT | 20 | 4277 |
| HSV1-UL30-1340 | + | CAGCTCGTTCAGGTGGGATT | 20 | 4278 |
| HSV1-UL30-1341 | + | AGCTCGTTCAGGTGGGATTC | 20 | 4279 |
| HSV1-UL30-1342 | + | GCTCGTTCAGGTGGGATTCG | 20 | 4280 |
| HSV1-UL30-1343 | + | CGTTCAGGTGGGATTCGGGG | 20 | 4281 |
| HSV1-UL30-1344 | + | GTGGGATTCGGGGAGGTCGC | 20 | 4282 |
| HSV1-UL30-1345 | + | CGCAGGAACCGAGCGAAAAC | 20 | 4283 |
| HSV1-UL30-1346 | + | AGGAACCGAGCGAAAACAGG | 20 | 4284 |
| HSV1-UL30-1347 | + | AAAACAGGAGGACGTGCTCC | 20 | 4285 |
| HSV1-UL30-1348 | + | AAACAGGAGGACGTGCTCCA | 20 | 4286 |
| HSV1-UL30-1349 | + | CAGGAGGACGTGCTCCAGGG | 20 | 4287 |
| HSV1-UL30-1350 | + | GAGGACGTGCTCCAGGGCGG | 20 | 4288 |
| HSV1-UL30-1351 | + | GACGTGCTCCAGGGCGGTGG | 20 | 4289 |
| HSV1-UL30-1352 | + | GCTCCAGGGCGGTGGTGGAC | 20 | 4290 |
| HSV1-UL30-1353 | + | GGACAGGTCGTAGAGCAGAC | 20 | 4291 |
| HSV1-UL30-1354 | + | CGTAGAGCAGACAGGATATC | 20 | 4292 |
| HSV1-UL30-1355 | + | GACAGGATATCTGGATGACC | 20 | 4293 |
| HSV1-UL30-1356 | + | ATCTGGATGACCAGGTCCTC | 20 | 4294 |
| HSV1-UL30-1357 | + | TCTGGATGACCAGGTCCTCC | 20 | 4295 |
| HSV1-UL30-1358 | + | GACCAGGTCCTCCGGGTGCC | 20 | 4296 |
| HSV1-UL30-1359 | + | TCCTCCGGGTGCCCGGCCAC | 20 | 4297 |
| HSV1-UL30-1360 | + | CGGGTGCCCGGCCACCGGAA | 20 | 4298 |
| HSV1-UL30-1361 | + | AAGCACATGAGCTTGTATGC | 20 | 4299 |
| HSV1-UL30-1362 | + | ACATGAGCTTGTATGCCGGT | 20 | 4300 |
| HSV1-UL30-1363 | + | GTCGCTCATGCCCCCCTCGA | 20 | 4301 |
| HSV1-UL30-1364 | + | TCATGCCCCCCTCGATGGCC | 20 | 4302 |
| HSV1-UL30-1365 | + | ACAGTTAAACTCGACGTCGC | 20 | 4303 |
| HSV1-UL30-1366 | + | GACGTCGCTGGATGTCCCGA | 20 | 4304 |
| HSV1-UL30-1367 | + | CTGGATGTCCCGAAGGCCAT | 20 | 4305 |
| HSV1-UL30-1368 | + | TGGATGTCCCGAAGGCCATC | 20 | 4306 |
| HSV1-UL30-1369 | + | GGATGTCCCGAAGGCCATCG | 20 | 4307 |
| HSV1-UL30-1370 | + | CCGAAGGCCATCGGGGCCGC | 20 | 4308 |
| HSV1-UL30-1371 | + | AGGCCATCGGGGCCGCCGGC | 20 | 4309 |
| HSV1-UL30-1372 | + | GGCCATCGGGGCCGCCGGCT | 20 | 4310 |
| HSV1-UL30-1373 | + | GCTGGGCTAGCGTGTTGTTC | 20 | 4311 |
| HSV1-UL30-1374 | + | GCTAGCGTGTTGTTCCGGCC | 20 | 4312 |
| HSV1-UL30-1375 | + | TGTTCCGGCCCGGTTTGAGA | 20 | 4313 |
| HSV1-UL30-1376 | + | TTTGAGACGGTACCAGCCGA | 20 | 4314 |
| HSV1-UL30-1377 | + | CCAGCCGAAGGTGACGAACC | 20 | 4315 |
| HSV1-UL30-1378 | + | CAGCCGAAGGTGACGAACCC | 20 | 4316 |
| HSV1-UL30-1379 | + | AGCCGAAGGTGACGAACCCG | 20 | 4317 |
| HSV1-UL30-1380 | + | TGACGAACCCGGGGTTGTCC | 20 | 4318 |
| HSV1-UL30-1381 | + | CGGGGTTGTCCAGGATGAAC | 20 | 4319 |
| HSV1-UL30-1382 | + | GGGGTTGTCCAGGATGAACC | 20 | 4320 |
| HSV1-UL30-1383 | + | GTTGTCCAGGATGAACCGGG | 20 | 4321 |
| HSV1-UL30-1384 | + | GTCCAGGATGAACCGGGTGG | 20 | 4322 |
| HSV1-UL30-1385 | + | CCCACCCTCGTACTTCTTGA | 20 | 4323 |
| HSV1-UL30-1386 | + | CCCTCGTACTTCTTGATGGC | 20 | 4324 |
| HSV1-UL30-1387 | + | CCTCGTACTTCTTGATGGCC | 20 | 4325 |
| HSV1-UL30-1388 | + | CCGGGCAGAAGTTGTCGCAC | 20 | 4326 |
| HSV1-UL30-1389 | + | ACGACAGCACACGCCCGCTT | 20 | 4327 |
| HSV1-UL30-1390 | + | GCCCGCTTCGGACGTAGACG | 20 | 4328 |
| HSV1-UL30-1391 | + | GTAGACGCGGTAAAACAGAG | 20 | 4329 |
| HSV1-UL30-1392 | + | TAGACGCGGTAAAACAGAGC | 20 | 4330 |
| HSV1-UL30-1393 | + | AGACGCGGTAAAACAGAGCG | 20 | 4331 |
| HSV1-UL30-1394 | + | CGTCTCGTAGTAGTACACGT | 20 | 4332 |
| HSV1-UL30-1395 | + | CCACCACCTCCGCCTCGAAG | 20 | 4333 |
| HSV1-UL30-1396 | + | CTCCGCCTCGAAGTGGTCCG | 20 | 4334 |
| HSV1-UL30-1397 | + | AGTGGTCCGCGGAGATGCCG | 20 | 4335 |
| HSV1-UL30-1398 | + | ATGCCGCGGAACGACGCGCC | 20 | 4336 |
| HSV1-UL30-1399 | + | TGCCGCGGAACGACGCGCCC | 20 | 4337 |
| HSV1-UL30-1400 | + | GCCGCGGAACGACGCGCCCG | 20 | 4338 |
| HSV1-UL30-1401 | + | ACGCGCCCGGGGACTCGCGC | 20 | 4339 |
| HSV1-UL30-1402 | + | CGCGCCCGGGGACTCGCGCA | 20 | 4340 |
| HSV1-UL30-1403 | + | CGGGGACTCGCGCAGGGCCG | 20 | 4341 |
| HSV1-UL30-1404 | + | ATGCGCTCGCAGAGATCTCG | 20 | 4342 |
| HSV1-UL30-1405 | + | TGCGCTCGCAGAGATCTCGT | 20 | 4343 |
| HSV1-UL30-1406 | + | GCGCTCGCAGAGATCTCGTG | 20 | 4344 |
| HSV1-UL30-1407 | + | CGCAGAGATCTCGTGGGGCG | 20 | 4345 |
| HSV1-UL30-1408 | + | CTCGTGGGGCGCGGCATTGT | 20 | 4346 |
| HSV1-UL30-1409 | + | CCGCGTGCCGTAAACGTGAA | 20 | 4347 |
| HSV1-UL30-1410 | + | CGTAAACGTGAACGGCCACC | 20 | 4348 |
| HSV1-UL30-1411 | + | AAACGTGAACGGCCACCCGG | 20 | 4349 |
| HSV1-UL30-1412 | + | ACGGCCACCCGGTGGCCTTC | 20 | 4350 |
| HSV1-UL30-1413 | + | CCCGGTGGCCTTCCGGAGTC | 20 | 4351 |
| HSV1-UL30-1414 | + | GGCCTTCCGGAGTCAGGCCC | 20 | 4352 |
| HSV1-UL30-1415 | + | CAGGCCCAGGAGCGTGATGA | 20 | 4353 |
| HSV1-UL30-1416 | + | AGCGTGATGACGGTCCCCGT | 20 | 4354 |
| HSV1-UL30-1417 | + | GACGGTCCCCGTCGGTGTGA | 20 | 4355 |
| HSV1-UL30-1418 | + | TGGCGTCCATAAACCGCGCG | 20 | 4356 |
| HSV1-UL30-1419 | + | GGCGTCCATAAACCGCGCGT | 20 | 4357 |
| HSV1-UL30-1420 | + | GCGTCCATAAACCGCGCGTG | 20 | 4358 |
| HSV1-UL30-1421 | + | CGTCCATAAACCGCGCGTGG | 20 | 4359 |
| HSV1-UL30-1422 | + | TTCCTCCGCCGTAGCGC | 17 | 4360 |
| HSV1-UL30-1423 | + | CACCGCCCCGAACCCTG | 17 | 4361 |
| HSV1-UL30-1424 | + | CCCCGAACCCTGCGGTC | 17 | 4362 |
| HSV1-UL30-1425 | + | TGCGGTCCGGAGCCGCG | 17 | 4363 |
| HSV1-UL30-1426 | + | CGCGCGGCCACGTCGTC | 17 | 4364 |
| HSV1-UL30-1427 | + | GCGCGGCCACGTCGTCC | 17 | 4365 |
| HSV1-UL30-1428 | + | CGCGGCCACGTCGTCCG | 17 | 4366 |
| HSV1-UL30-1429 | + | GCGGCCACGTCGTCCGG | 17 | 4367 |
| HSV1-UL30-1430 | + | CGGCCACGTCGTCCGGG | 17 | 4368 |
| HSV1-UL30-1431 | + | CGGGGGGTGCCACACTT | 17 | 4369 |
| HSV1-UL30-1432 | + | GGGGGGTGCCACACTTC | 17 | 4370 |
| HSV1-UL30-1433 | + | CCTTTTTAACAGACTCT | 17 | 4371 |
| HSV1-UL30-1434 | + | CAGACTCTCGGTGATCT | 17 | 4372 |
| HSV1-UL30-1435 | + | TGGCGTTATTCCCAAAC | 17 | 4373 |
| HSV1-UL30-1436 | + | GGCGTTATTCCCAAACA | 17 | 4374 |
| HSV1-UL30-1437 | + | CGCACGCCGCCCCCAAC | 17 | 4375 |
| HSV1-UL30-1438 | + | ACGCCGCCCCCAACAGG | 17 | 4376 |
| HSV1-UL30-1439 | + | CGCCGCCCCCAACAGGT | 17 | 4377 |
| HSV1-UL30-1440 | + | AGTAATAGTCCGTGTTC | 17 | 4378 |
| HSV1-UL30-1441 | + | GTAATAGTCCGTGTTCA | 17 | 4379 |
| HSV1-UL30-1442 | + | TGTTCAGGGCGACGCCG | 17 | 4380 |
| HSV1-UL30-1443 | + | GTTCAGGGCGACGCCGT | 17 | 4381 |
| HSV1-UL30-1444 | + | GGCGACGCCGTGGGCAA | 17 | 4382 |
| HSV1-UL30-1445 | + | GTGGGCAATGGCGTATG | 17 | 4383 |
| HSV1-UL30-1446 | + | TGGGCAATGGCGTATGC | 17 | 4384 |
| HSV1-UL30-1447 | + | GGCGTATGCGGGATCCT | 17 | 4385 |
| HSV1-UL30-1448 | + | GGGATCCTCGGCCAGCT | 17 | 4386 |
| HSV1-UL30-1449 | + | CGGACACCAGCAGCTTG | 17 | 4387 |
| HSV1-UL30-1450 | + | GGACACCAGCAGCTTGC | 17 | 4388 |
| HSV1-UL30-1451 | + | GACACCAGCAGCTTGCG | 17 | 4389 |
| HSV1-UL30-1452 | + | CAGCAGCTTGCGGGGCT | 17 | 4390 |
| HSV1-UL30-1453 | + | GGCTTGGACGCGCCTCC | 17 | 4391 |
| HSV1-UL30-1454 | + | GCTTGGACGCGCCTCCC | 17 | 4392 |
| HSV1-UL30-1455 | + | CTTGGACGCGCCTCCCG | 17 | 4393 |
| HSV1-UL30-1456 | + | TTGGACGCGCCTCCCGG | 17 | 4394 |
| HSV1-UL30-1457 | + | TGGACGCGCCTCCCGGG | 17 | 4395 |
| HSV1-UL30-1458 | + | CGCGCCTCCCGGGGGGT | 17 | 4396 |
| HSV1-UL30-1459 | + | CCTCCCGGGGGGTCGGC | 17 | 4397 |
| HSV1-UL30-1460 | + | GGGGGGTCGGCAGGCGA | 17 | 4398 |
| HSV1-UL30-1461 | + | CAGGCGACGGCGTCTCC | 17 | 4399 |
| HSV1-UL30-1462 | + | AGGCGACGGCGTCTCCC | 17 | 4400 |
| HSV1-UL30-1463 | + | GGCGACGGCGTCTCCCG | 17 | 4401 |
| HSV1-UL30-1464 | + | GCGACGGCGTCTCCCGG | 17 | 4402 |
| HSV1-UL30-1465 | + | CGTCTCCCGGGGGCGCT | 17 | 4403 |
| HSV1-UL30-1466 | + | TCCCGGGGGCGCTTGGC | 17 | 4404 |
| HSV1-UL30-1467 | + | CCCGGGGGCGCTTGGCC | 17 | 4405 |
| HSV1-UL30-1468 | + | CCGGGGGCGCTTGGCCG | 17 | 4406 |
| HSV1-UL30-1469 | + | GGGGCGCTTGGCCGGGG | 17 | 4407 |
| HSV1-UL30-1470 | + | GGGCGCTTGGCCGGGGA | 17 | 4408 |
| HSV1-UL30-1471 | + | GCTTGGCCGGGGAGGGC | 17 | 4409 |
| HSV1-UL30-1472 | + | CTTGGCCGGGGAGGGCA | 17 | 4410 |
| HSV1-UL30-1473 | + | CGGGGAGGGCAGGGCCG | 17 | 4411 |
| HSV1-UL30-1474 | + | GGGGAGGGCAGGGCCGC | 17 | 4412 |
| HSV1-UL30-1475 | + | GGGAGGGCAGGGCCGCG | 17 | 4413 |
| HSV1-UL30-1476 | + | GGAGGGCAGGGCCGCGG | 17 | 4414 |
| HSV1-UL30-1477 | + | GAGGGCAGGGCCGCGGG | 17 | 4415 |
| HSV1-UL30-1478 | + | AGGGCAGGGCCGCGGGG | 17 | 4416 |
| HSV1-UL30-1479 | + | GGGCAGGGCCGCGGGGG | 17 | 4417 |
| HSV1-UL30-1480 | + | CAGGGCCGCGGGGGGGG | 17 | 4418 |
| HSV1-UL30-1481 | + | AGGGCCGCGGGGGGGGC | 17 | 4419 |
| HSV1-UL30-1482 | + | GGGGCGGGCTCGTCCCC | 17 | 4420 |
| HSV1-UL30-1483 | + | GGGCGGGCTCGTCCCCT | 17 | 4421 |
| HSV1-UL30-1484 | + | GGCGGGCTCGTCCCCTG | 17 | 4422 |
| HSV1-UL30-1485 | + | GGGCTCGTCCCCTGGGG | 17 | 4423 |
| HSV1-UL30-1486 | + | CTCGTCCCCTGGGGCGG | 17 | 4424 |
| HSV1-UL30-1487 | + | CGGCGGCGTCTAGCTCG | 17 | 4425 |
| HSV1-UL30-1488 | + | CGGCGTCTAGCTCGCGG | 17 | 4426 |
| HSV1-UL30-1489 | + | GGCGTCTAGCTCGCGGA | 17 | 4427 |
| HSV1-UL30-1490 | + | GTCTAGCTCGCGGAGGG | 17 | 4428 |
| HSV1-UL30-1491 | + | CCGTCTCCTCTACCTCG | 17 | 4429 |
| HSV1-UL30-1492 | + | CGTCTCCTCTACCTCGC | 17 | 4430 |
| HSV1-UL30-1493 | + | CCTCTACCTCGCGGGTC | 17 | 4431 |
| HSV1-UL30-1494 | + | CTCTACCTCGCGGGTCT | 17 | 4432 |
| HSV1-UL30-1495 | + | TGGGCCACGATCACGTA | 17 | 4433 |
| HSV1-UL30-1496 | + | GGGCCACGATCACGTAC | 17 | 4434 |
| HSV1-UL30-1497 | + | CGATCACGTACGGGATC | 17 | 4435 |
| HSV1-UL30-1498 | + | CGGGATCCGGTCCTTGA | 17 | 4436 |
| HSV1-UL30-1499 | + | ATCCGGTCCTTGATGGA | 17 | 4437 |
| HSV1-UL30-1500 | + | TCCGGTCCTTGATGGAC | 17 | 4438 |
| HSV1-UL30-1501 | + | TGGACGGGACCTGCGCG | 17 | 4439 |
| HSV1-UL30-1502 | + | ACGGGACCTGCGCGCGG | 17 | 4440 |
| HSV1-UL30-1503 | + | CGGGACCTGCGCGCGGC | 17 | 4441 |
| HSV1-UL30-1504 | + | GCTTGTAATACACCGTC | 17 | 4442 |
| HSV1-UL30-1505 | + | TGTAATACACCGTCAGG | 17 | 4443 |
| HSV1-UL30-1506 | + | GTAATACACCGTCAGGT | 17 | 4444 |
| HSV1-UL30-1507 | + | ACACCGTCAGGTGGGCC | 17 | 4445 |
| HSV1-UL30-1508 | + | GTGGGCCAGGCGCTTGT | 17 | 4446 |
| HSV1-UL30-1509 | + | TTGTTGGTGTACGCGCG | 17 | 4447 |
| HSV1-UL30-1510 | + | TGTTGGTGTACGCGCGC | 17 | 4448 |
| HSV1-UL30-1511 | + | CGGGTGTCTGCTCAGTT | 17 | 4449 |
| HSV1-UL30-1512 | + | GTGTCTGCTCAGTTCGG | 17 | 4450 |
| HSV1-UL30-1513 | + | TGCTCAGTTCGGCGGTG | 17 | 4451 |
| HSV1-UL30-1514 | + | CGGTGAGGACAAAGTCC | 17 | 4452 |
| HSV1-UL30-1515 | + | TCCTGGATGTCCCTCTC | 17 | 4453 |
| HSV1-UL30-1516 | + | CCTGGATGTCCCTCTCC | 17 | 4454 |
| HSV1-UL30-1517 | + | GATGTCCCTCTCCGGGT | 17 | 4455 |
| HSV1-UL30-1518 | + | GGTCGGTGATGCGCCGA | 17 | 4456 |
| HSV1-UL30-1519 | + | GTCGGTGATGCGCCGAT | 17 | 4457 |
| HSV1-UL30-1520 | + | GCCGATGGGCGTCTACG | 17 | 4458 |
| HSV1-UL30-1521 | + | ATGGGCGTCTACGAGGA | 17 | 4459 |
| HSV1-UL30-1522 | + | GAACGCCTGCAGTCCCT | 17 | 4460 |
| HSV1-UL30-1523 | + | AACGCCTGCAGTCCCTC | 17 | 4461 |
| HSV1-UL30-1524 | + | CCTGCAGTCCCTCGGGC | 17 | 4462 |
| HSV1-UL30-1525 | + | CTGCAGTCCCTCGGGCA | 17 | 4463 |
| HSV1-UL30-1526 | + | TGCAGTCCCTCGGGCAG | 17 | 4464 |
| HSV1-UL30-1527 | + | CGCCAGCCACTCCTCCG | 17 | 4465 |
| HSV1-UL30-1528 | + | GCCAGCCACTCCTCCGC | 17 | 4466 |
| HSV1-UL30-1529 | + | CCAGCCACTCCTCCGCG | 17 | 4467 |
| HSV1-UL30-1530 | + | CTCCTCCGCGGGGCGCT | 17 | 4468 |
| HSV1-UL30-1531 | + | GGGGCGCTCGGCTAACG | 17 | 4469 |
| HSV1-UL30-1532 | + | GCGCTCGGCTAACGCGG | 17 | 4470 |
| HSV1-UL30-1533 | + | TAACGCGGCGGCCGCTC | 17 | 4471 |
| HSV1-UL30-1534 | + | GGCGGCCGCTCCGGAGA | 17 | 4472 |
| HSV1-UL30-1535 | + | TATCGTCGTAAAACAGC | 17 | 4473 |
| HSV1-UL30-1536 | + | AAAACAGCAGGTCGACC | 17 | 4474 |
| HSV1-UL30-1537 | + | AAACAGCAGGTCGACCA | 17 | 4475 |
| HSV1-UL30-1538 | + | CAGGTCGACCAGGGCCC | 17 | 4476 |
| HSV1-UL30-1539 | + | GTCGACCAGGGCCCTGG | 17 | 4477 |
| HSV1-UL30-1540 | + | CCAGGGCCCTGGAGGTG | 17 | 4478 |
| HSV1-UL30-1541 | + | GCCGATGTACTTTTTCT | 17 | 4479 |
| HSV1-UL30-1542 | + | GGCGATCAGCAGCAGCT | 17 | 4480 |
| HSV1-UL30-1543 | + | TTCGCACTCGAGTTTGA | 17 | 4481 |
| HSV1-UL30-1544 | + | TCGCACTCGAGTTTGAT | 17 | 4482 |
| HSV1-UL30-1545 | + | CGCACTCGAGTTTGATG | 17 | 4483 |
| HSV1-UL30-1546 | + | GCACTCGAGTTTGATGG | 17 | 4484 |
| HSV1-UL30-1547 | + | CACTCGAGTTTGATGGG | 17 | 4485 |
| HSV1-UL30-1548 | + | ACAGCGCGCGCGAGATG | 17 | 4486 |
| HSV1-UL30-1549 | + | CGCCATCTTGTCGCCCA | 17 | 4487 |
| HSV1-UL30-1550 | + | GCCCACGGCCGTCAGCC | 17 | 4488 |
| HSV1-UL30-1551 | + | CACGGCCGTCAGCCCGG | 17 | 4489 |
| HSV1-UL30-1552 | + | TCAGCCCGGCGGCCGTG | 17 | 4490 |
| HSV1-UL30-1553 | + | CGGCGGCCGTGAGGCCG | 17 | 4491 |
| HSV1-UL30-1554 | + | GCGGCACAGCACAAAGA | 17 | 4492 |
| HSV1-UL30-1555 | + | CCCGTAGATGATGCGCA | 17 | 4493 |
| HSV1-UL30-1556 | + | GATGATGCGCATGGAAT | 17 | 4494 |
| HSV1-UL30-1557 | + | ATGATGCGCATGGAATA | 17 | 4495 |
| HSV1-UL30-1558 | + | GCGCATGGAATAGGGCC | 17 | 4496 |
| HSV1-UL30-1559 | + | CGCATGGAATAGGGCCC | 17 | 4497 |
| HSV1-UL30-1560 | + | GCATGGAATAGGGCCCG | 17 | 4498 |
| HSV1-UL30-1561 | + | CATGGAATAGGGCCCGG | 17 | 4499 |
| HSV1-UL30-1562 | + | CCCGGGGGCGCGCATGT | 17 | 4500 |
| HSV1-UL30-1563 | + | CGCATGTCGGCCGCCTC | 17 | 4501 |
| HSV1-UL30-1564 | + | GCATGTCGGCCGCCTCC | 17 | 4502 |
| HSV1-UL30-1565 | + | GGCCGCCTCCGGGAAAT | 17 | 4503 |
| HSV1-UL30-1566 | + | CCTCCGGGAAATCGGCC | 17 | 4504 |
| HSV1-UL30-1567 | + | GGCCAGGAGCTGTTCGA | 17 | 4505 |
| HSV1-UL30-1568 | + | AGGCCGCCCAGCGCGCG | 17 | 4506 |
| HSV1-UL30-1569 | + | GCGCGTGGACGTACTCG | 17 | 4507 |
| HSV1-UL30-1570 | + | CGCGTGGACGTACTCGC | 17 | 4508 |
| HSV1-UL30-1571 | + | TCGCGAGCAGCATCTCG | 17 | 4509 |
| HSV1-UL30-1572 | + | CAGCATCTCGCGGCCGA | 17 | 4510 |
| HSV1-UL30-1573 | + | GATGGTCGTCACCGTCG | 17 | 4511 |
| HSV1-UL30-1574 | + | CCGTCGCGGCAACGTGC | 17 | 4512 |
| HSV1-UL30-1575 | + | GCGGCAACGTGCAGGCA | 17 | 4513 |
| HSV1-UL30-1576 | + | CAACGTGCAGGCACGGC | 17 | 4514 |
| HSV1-UL30-1577 | + | GTTACACACGACCTTGA | 17 | 4515 |
| HSV1-UL30-1578 | + | ACACACGACCTTGATGG | 17 | 4516 |
| HSV1-UL30-1579 | + | CGGCCTGCTGCTTGTCC | 17 | 4517 |
| HSV1-UL30-1580 | + | CTGCTTGTCCAGGAGCA | 17 | 4518 |
| HSV1-UL30-1581 | + | CAGGAGCACGGCCTCCT | 17 | 4519 |
| HSV1-UL30-1582 | + | AGGAGCACGGCCTCCTC | 17 | 4520 |
| HSV1-UL30-1583 | + | GGAGCACGGCCTCCTCG | 17 | 4521 |
| HSV1-UL30-1584 | + | CCTCCTCGGGGCTGCTC | 17 | 4522 |
| HSV1-UL30-1585 | + | CTCCTCGGGGCTGCTCT | 17 | 4523 |
| HSV1-UL30-1586 | + | TCCTCGGGGCTGCTCTG | 17 | 4524 |
| HSV1-UL30-1587 | + | TCTGGGGAATCCGCGAG | 17 | 4525 |
| HSV1-UL30-1588 | + | GCGGATCTGCTTTCGCA | 17 | 4526 |
| HSV1-UL30-1589 | + | TGGCGAGCCAGTCCCGC | 17 | 4527 |
| HSV1-UL30-1590 | + | CGAGCCAGTCCCGCAGG | 17 | 4528 |
| HSV1-UL30-1591 | + | CCCGCAGGAGGATGCTG | 17 | 4529 |
| HSV1-UL30-1592 | + | GCAGGAGGATGCTGAGG | 17 | 4530 |
| HSV1-UL30-1593 | + | CCCCCACCTCGATCTCC | 17 | 4531 |
| HSV1-UL30-1594 | + | AGTCCTTGCCCGCCTCC | 17 | 4532 |
| HSV1-UL30-1595 | + | CAGGTGCGCCACTGCGT | 17 | 4533 |
| HSV1-UL30-1596 | + | CCACTGCGTCGGCCCTC | 17 | 4534 |
| HSV1-UL30-1597 | + | CACTGCGTCGGCCCTCA | 17 | 4535 |
| HSV1-UL30-1598 | + | AGAGCGTGCTGAAGCAC | 17 | 4536 |
| HSV1-UL30-1599 | + | TGCTGAAGCACAGGTTG | 17 | 4537 |
| HSV1-UL30-1600 | + | GCTGAAGCACAGGTTGT | 17 | 4538 |
| HSV1-UL30-1601 | + | AGCACAGGTTGTGGGCC | 17 | 4539 |
| HSV1-UL30-1602 | + | GTGGGCCTGGATGATGC | 17 | 4540 |
| HSV1-UL30-1603 | + | TGGGCCTGGATGATGCT | 17 | 4541 |
| HSV1-UL30-1604 | + | GGGCCTGGATGATGCTG | 17 | 4542 |
| HSV1-UL30-1605 | + | GGATGATGCTGGGGTAC | 17 | 4543 |
| HSV1-UL30-1606 | + | GATGCTGGGGTACAGGC | 17 | 4544 |
| HSV1-UL30-1607 | + | AAAGTCGAACACCACCA | 17 | 4545 |
| HSV1-UL30-1608 | + | AAGTCGAACACCACCAC | 17 | 4546 |
| HSV1-UL30-1609 | + | AGTCGAACACCACCACG | 17 | 4547 |
| HSV1-UL30-1610 | + | GGGGTTCACGTGAAACC | 17 | 4548 |
| HSV1-UL30-1611 | + | CACGTGAAACCCGGAAG | 17 | 4549 |
| HSV1-UL30-1612 | + | ACGTGAAACCCGGAAGT | 17 | 4550 |
| HSV1-UL30-1613 | + | CGTGAAACCCGGAAGTG | 17 | 4551 |
| HSV1-UL30-1614 | + | ACCCGGAAGTGGGGTCA | 17 | 4552 |
| HSV1-UL30-1615 | + | AGTGGGGTCAAGGACCC | 17 | 4553 |
| HSV1-UL30-1616 | + | CAAGGACCCTGGCCCCC | 17 | 4554 |
| HSV1-UL30-1617 | + | GTACCCCACGTGCCTGC | 17 | 4555 |
| HSV1-UL30-1618 | + | CCCCACGTGCCTGCCGG | 17 | 4556 |
| HSV1-UL30-1619 | + | GTCTCCCGCGCGCCCTC | 17 | 4557 |
| HSV1-UL30-1620 | + | TCCTCCCCCTCCTCCTC | 17 | 4558 |
| HSV1-UL30-1621 | + | GCCGCTCCTCGTCCTCC | 17 | 4559 |
| HSV1-UL30-1622 | + | CCGCTCCTCGTCCTCCC | 17 | 4560 |
| HSV1-UL30-1623 | + | CTCGTCCTCCCGGGCTG | 17 | 4561 |
| HSV1-UL30-1624 | + | TCCTCCCGGGCTGCGGC | 17 | 4562 |
| HSV1-UL30-1625 | + | GGCTGCGGCCGGACGCT | 17 | 4563 |
| HSV1-UL30-1626 | + | GCTGCGGCCGGACGCTT | 17 | 4564 |
| HSV1-UL30-1627 | + | CTTGGGCGCCTCCCCCC | 17 | 4565 |
| HSV1-UL30-1628 | + | CGCCCCTAAATCGCCCC | 17 | 4566 |
| HSV1-UL30-1629 | + | GCCCCTAAATCGCCCCT | 17 | 4567 |
| HSV1-UL30-1630 | + | AATCGCCCCTGGGTGTC | 17 | 4568 |
| HSV1-UL30-1631 | + | GCAGAATAAAGCCCTTC | 17 | 4569 |
| HSV1-UL30-1632 | + | AATAAAGCCCTTCTGGT | 17 | 4570 |
| HSV1-UL30-1633 | + | AGCCCTTCTGGTCGGCC | 17 | 4571 |
| HSV1-UL30-1634 | + | GGTCGGCCAGGCGCAGC | 17 | 4572 |
| HSV1-UL30-1635 | + | GCAGGCACGTAAAGACG | 17 | 4573 |
| HSV1-UL30-1636 | + | TAAAGACGCGGATCTGC | 17 | 4574 |
| HSV1-UL30-1637 | + | CTGCTGGCCGTCGTAGA | 17 | 4575 |
| HSV1-UL30-1638 | + | GGCCGTCGTAGATGGTG | 17 | 4576 |
| HSV1-UL30-1639 | + | GCCGTCGTAGATGGTGC | 17 | 4577 |
| HSV1-UL30-1640 | + | ACCCGCCAAGCGCGCGA | 17 | 4578 |
| HSV1-UL30-1641 | + | CGGCCGAGAGCTCCAGA | 17 | 4579 |
| HSV1-UL30-1642 | + | GGCCGAGAGCTCCAGAT | 17 | 4580 |
| HSV1-UL30-1643 | + | GCCGAGAGCTCCAGATG | 17 | 4581 |
| HSV1-UL30-1644 | + | AAAACTTAAAAAACAGC | 17 | 4582 |
| HSV1-UL30-1645 | + | ACAGCTGGCCCACCAGC | 17 | 4583 |
| HSV1-UL30-1646 | + | CAGCTGGCCCACCAGCA | 17 | 4584 |
| HSV1-UL30-1647 | + | GATCACCCCGCGTTGCG | 17 | 4585 |
| HSV1-UL30-1648 | + | ATCACCCCGCGTTGCGC | 17 | 4586 |
| HSV1-UL30-1649 | + | CCCGCGTTGCGCGGGCC | 17 | 4587 |
| HSV1-UL30-1650 | + | GCGTTGCGCGGGCCCGG | 17 | 4588 |
| HSV1-UL30-1651 | + | GGGCCCGGCGGCGTAGT | 17 | 4589 |
| HSV1-UL30-1652 | + | CCCGGCGGCGTAGTAGG | 17 | 4590 |
| HSV1-UL30-1653 | + | CCGGCGGCGTAGTAGGC | 17 | 4591 |
| HSV1-UL30-1654 | + | CGGCGGCGTAGTAGGCG | 17 | 4592 |
| HSV1-UL30-1655 | + | GGATGTCGCGATAGCTC | 17 | 4593 |
| HSV1-UL30-1656 | + | CCTTCTTCTTGTCCTTC | 17 | 4594 |
| HSV1-UL30-1657 | + | CTTCTTGTCCTTCAGGA | 17 | 4595 |
| HSV1-UL30-1658 | + | GTCCTTCAGGACGGCTT | 17 | 4596 |
| HSV1-UL30-1659 | + | CAGGACGGCTTCGGCCA | 17 | 4597 |
| HSV1-UL30-1660 | + | CGAGAGCTTGATCTTGT | 17 | 4598 |
| HSV1-UL30-1661 | + | TTATCTTGCTGCGCTTC | 17 | 4599 |
| HSV1-UL30-1662 | + | TGCTGCGCTTCTGGAAG | 17 | 4600 |
| HSV1-UL30-1663 | + | GCTTCTGGAAGTGGCTC | 17 | 4601 |
| HSV1-UL30-1664 | + | ACACGCGAAACACGCCC | 17 | 4602 |
| HSV1-UL30-1665 | + | CGCCCCGGCCGTTCATG | 17 | 4603 |
| HSV1-UL30-1666 | + | TGCGGCCGTACCCGTCC | 17 | 4604 |
| HSV1-UL30-1667 | + | GCGGCCGTACCCGTCCA | 17 | 4605 |
| HSV1-UL30-1668 | + | CGGCCGTACCCGTCCAG | 17 | 4606 |
| HSV1-UL30-1669 | + | GGCCGTACCCGTCCAGG | 17 | 4607 |
| HSV1-UL30-1670 | + | GTAAATGTCCGTCAGCT | 17 | 4608 |
| HSV1-UL30-1671 | + | CAGCTTGGCCAGCAAGA | 17 | 4609 |
| HSV1-UL30-1672 | + | AGCTTGGCCAGCAAGAA | 17 | 4610 |
| HSV1-UL30-1673 | + | GTTGATGATGTTGTACC | 17 | 4611 |
| HSV1-UL30-1674 | + | GTACCCGGTCACGAACT | 17 | 4612 |
| HSV1-UL30-1675 | + | TACCCGGTCACGAACTC | 17 | 4613 |
| HSV1-UL30-1676 | + | ACCCGGTCACGAACTCG | 17 | 4614 |
| HSV1-UL30-1677 | + | GGCCGTACTGTTTCACA | 17 | 4615 |
| HSV1-UL30-1678 | + | GCCGTACTGTTTCACAA | 17 | 4616 |
| HSV1-UL30-1679 | + | TTTCACAAGGGTCATGA | 17 | 4617 |
| HSV1-UL30-1680 | + | GTCGAATTCCAGAACCA | 17 | 4618 |
| HSV1-UL30-1681 | + | TCGAATTCCAGAACCAC | 17 | 4619 |
| HSV1-UL30-1682 | + | TTCCAGAACCACGGGCG | 17 | 4620 |
| HSV1-UL30-1683 | + | TCCAGAACCACGGGCGT | 17 | 4621 |
| HSV1-UL30-1684 | + | GAACCACGGGCGTGGGC | 17 | 4622 |
| HSV1-UL30-1685 | + | GGGCGTGGGCAGGCCCC | 17 | 4623 |
| HSV1-UL30-1686 | + | TGGCCGCCAGCTCGTTC | 17 | 4624 |
| HSV1-UL30-1687 | + | CCGCCAGCTCGTTCAGG | 17 | 4625 |
| HSV1-UL30-1688 | + | CGCCAGCTCGTTCAGGT | 17 | 4626 |
| HSV1-UL30-1689 | + | CTCGTTCAGGTGGGATT | 17 | 4627 |
| HSV1-UL30-1690 | + | TCGTTCAGGTGGGATTC | 17 | 4628 |
| HSV1-UL30-1691 | + | CGTTCAGGTGGGATTCG | 17 | 4629 |
| HSV1-UL30-1692 | + | TCAGGTGGGATTCGGGG | 17 | 4630 |
| HSV1-UL30-1693 | + | GGATTCGGGGAGGTCGC | 17 | 4631 |
| HSV1-UL30-1694 | + | AGGAACCGAGCGAAAAC | 17 | 4632 |
| HSV1-UL30-1695 | + | AACCGAGCGAAAACAGG | 17 | 4633 |
| HSV1-UL30-1696 | + | ACAGGAGGACGTGCTCC | 17 | 4634 |
| HSV1-UL30-1697 | + | CAGGAGGACGTGCTCCA | 17 | 4635 |
| HSV1-UL30-1698 | + | GAGGACGTGCTCCAGGG | 17 | 4636 |
| HSV1-UL30-1699 | + | GACGTGCTCCAGGGCGG | 17 | 4637 |
| HSV1-UL30-1700 | + | GTGCTCCAGGGCGGTGG | 17 | 4638 |
| HSV1-UL30-1701 | + | CCAGGGCGGTGGTGGAC | 17 | 4639 |
| HSV1-UL30-1702 | + | CAGGTCGTAGAGCAGAC | 17 | 4640 |
| HSV1-UL30-1703 | + | AGAGCAGACAGGATATC | 17 | 4641 |
| HSV1-UL30-1704 | + | AGGATATCTGGATGACC | 17 | 4642 |
| HSV1-UL30-1705 | + | TGGATGACCAGGTCCTC | 17 | 4643 |
| HSV1-UL30-1706 | + | GGATGACCAGGTCCTCC | 17 | 4644 |
| HSV1-UL30-1707 | + | CAGGTCCTCCGGGTGCC | 17 | 4645 |
| HSV1-UL30-1708 | + | TCCGGGTGCCCGGCCAC | 17 | 4646 |
| HSV1-UL30-1709 | + | GTGCCCGGCCACCGGAA | 17 | 4647 |
| HSV1-UL30-1710 | + | CACATGAGCTTGTATGC | 17 | 4648 |
| HSV1-UL30-1711 | + | TGAGCTTGTATGCCGGT | 17 | 4649 |
| HSV1-UL30-1712 | + | GCTCATGCCCCCCTCGA | 17 | 4650 |
| HSV1-UL30-1713 | + | TGCCCCCCTCGATGGCC | 17 | 4651 |
| HSV1-UL30-1714 | + | GTTAAACTCGACGTCGC | 17 | 4652 |
| HSV1-UL30-1715 | + | GTCGCTGGATGTCCCGA | 17 | 4653 |
| HSV1-UL30-1716 | + | GATGTCCCGAAGGCCAT | 17 | 4654 |
| HSV1-UL30-1717 | + | ATGTCCCGAAGGCCATC | 17 | 4655 |
| HSV1-UL30-1718 | + | TGTCCCGAAGGCCATCG | 17 | 4656 |
| HSV1-UL30-1719 | + | AAGGCCATCGGGGCCGC | 17 | 4657 |
| HSV1-UL30-1720 | + | CCATCGGGGCCGCCGGC | 17 | 4658 |
| HSV1-UL30-1721 | + | CATCGGGGCCGCCGGCT | 17 | 4659 |
| HSV1-UL30-1722 | + | GGGCTAGCGTGTTGTTC | 17 | 4660 |
| HSV1-UL30-1723 | + | AGCGTGTTGTTCCGGCC | 17 | 4661 |
| HSV1-UL30-1724 | + | TCCGGCCCGGTTTGAGA | 17 | 4662 |
| HSV1-UL30-1725 | + | GAGACGGTACCAGCCGA | 17 | 4663 |
| HSV1-UL30-1726 | + | GCCGAAGGTGACGAACC | 17 | 4664 |
| HSV1-UL30-1727 | + | CCGAAGGTGACGAACCC | 17 | 4665 |
| HSV1-UL30-1728 | + | CGAAGGTGACGAACCCG | 17 | 4666 |
| HSV1-UL30-1729 | + | CGAACCCGGGGTTGTCC | 17 | 4667 |
| HSV1-UL30-1730 | + | GGTTGTCCAGGATGAAC | 17 | 4668 |
| HSV1-UL30-1731 | + | GTTGTCCAGGATGAACC | 17 | 4669 |
| HSV1-UL30-1732 | + | GTCCAGGATGAACCGGG | 17 | 4670 |
| HSV1-UL30-1733 | + | CAGGATGAACCGGGTGG | 17 | 4671 |
| HSV1-UL30-1734 | + | ACCCTCGTACTTCTTGA | 17 | 4672 |
| HSV1-UL30-1735 | + | TCGTACTTCTTGATGGC | 17 | 4673 |
| HSV1-UL30-1736 | + | CGTACTTCTTGATGGCC | 17 | 4674 |
| HSV1-UL30-1737 | + | GGCAGAAGTTGTCGCAC | 17 | 4675 |
| HSV1-UL30-1738 | + | ACAGCACACGCCCGCTT | 17 | 4676 |
| HSV1-UL30-1739 | + | CGCTTCGGACGTAGACG | 17 | 4677 |
| HSV1-UL30-1740 | + | GACGCGGTAAAACAGAG | 17 | 4678 |
| HSV1-UL30-1741 | + | ACGCGGTAAAACAGAGC | 17 | 4679 |
| HSV1-UL30-1742 | + | CGCGGTAAAACAGAGCG | 17 | 4680 |
| HSV1-UL30-1743 | + | CTCGTAGTAGTACACGT | 17 | 4681 |
| HSV1-UL30-1744 | + | CCACCTCCGCCTCGAAG | 17 | 4682 |
| HSV1-UL30-1745 | + | CGCCTCGAAGTGGTCCG | 17 | 4683 |
| HSV1-UL30-1746 | + | GGTCCGCGGAGATGCCG | 17 | 4684 |
| HSV1-UL30-1747 | + | CCGCGGAACGACGCGCC | 17 | 4685 |
| HSV1-UL30-1748 | + | CGCGGAACGACGCGCCC | 17 | 4686 |
| HSV1-UL30-1749 | + | GCGGAACGACGCGCCCG | 17 | 4687 |
| HSV1-UL30-1750 | + | CGCCCGGGGACTCGCGC | 17 | 4688 |
| HSV1-UL30-1751 | + | GCCCGGGGACTCGCGCA | 17 | 4689 |
| HSV1-UL30-1752 | + | GGACTCGCGCAGGGCCG | 17 | 4690 |
| HSV1-UL30-1753 | + | CGCTCGCAGAGATCTCG | 17 | 4691 |
| HSV1-UL30-1754 | + | GCTCGCAGAGATCTCGT | 17 | 4692 |
| HSV1-UL30-1755 | + | CTCGCAGAGATCTCGTG | 17 | 4693 |
| HSV1-UL30-1756 | + | AGAGATCTCGTGGGGCG | 17 | 4694 |
| HSV1-UL30-1757 | + | GTGGGGCGCGGCATTGT | 17 | 4695 |
| HSV1-UL30-1758 | + | CGTGCCGTAAACGTGAA | 17 | 4696 |
| HSV1-UL30-1759 | + | AAACGTGAACGGCCACC | 17 | 4697 |
| HSV1-UL30-1760 | + | CGTGAACGGCCACCCGG | 17 | 4698 |
| HSV1-UL30-1761 | + | GCCACCCGGTGGCCTTC | 17 | 4699 |
| HSV1-UL30-1762 | + | GGTGGCCTTCCGGAGTC | 17 | 4700 |
| HSV1-UL30-1763 | + | CTTCCGGAGTCAGGCCC | 17 | 4701 |
| HSV1-UL30-1764 | + | GCCCAGGAGCGTGATGA | 17 | 4702 |
| HSV1-UL30-1765 | + | GTGATGACGGTCCCCGT | 17 | 4703 |
| HSV1-UL30-1766 | + | GGTCCCCGTCGGTGTGA | 17 | 4704 |
| HSV1-UL30-1767 | + | CGTCCATAAACCGCGCG | 17 | 4705 |
| HSV1-UL30-1768 | + | GTCCATAAACCGCGCGT | 17 | 4706 |
| HSV1-UL30-1769 | + | TCCATAAACCGCGCGTG | 17 | 4707 |
| HSV1-UL30-1770 | + | CCATAAACCGCGCGTGG | 17 | 4708 |

Table 2D) provides exemplary targeting domains for knocking out the UL30 gene selected according to the first tier parameters. The targeting domains are selected based on location within first 500 bp of the coding sequence of the UL30 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2D

| 1st Tier | | | Target Site | Seq |
|---|---|---|---|---|
| gRNA Name | DNA Strand | Targeting Domain | Length | ID |
| HSV1-UL30-237 | − | UGGCGGCGGCCCGCUGUCCC | 20 | 4709 |
| HSV1-UL30-1 | − | GGCGGCGGCCCGCUGUCCCC | 20 | 3232 |
| HSV1-UL30-239 | − | CGGCGGCCCGCUGUCCCCCG | 20 | 4710 |
| HSV1-UL30-2 | − | GGCGGCCCGCUGUCCCCCGG | 20 | 3233 |
| HSV1-UL30-241 | − | CCCGGAGGAAAGUCGGCGGC | 20 | 4711 |
| HSV1-UL30-242 | − | GUCGGCGGCCAGGGCGGCGU | 20 | 4712 |
| HSV1-UL30-243 | − | UUUUGCGCCCGCCGGCCCUC | 20 | 4713 |
| HSV1-UL30-11 | − | UUUGCGCCCGCCGGCCCUCG | 20 | 3125 |
| HSV1-UL30-245 | − | GCCGGCCCUCGCGGAGCCAG | 20 | 4714 |
| HSV1-UL30-12 | − | CCGGCCCUCGCGGAGCCAGC | 20 | 3240 |
| HSV1-UL30-13 | − | CGGCCCUCGCGGAGCCAGCC | 20 | 3241 |
| HSV1-UL30-248 | − | GCCGGGGACCCCCGCCUUGU | 20 | 4715 |
| HSV1-UL30-249 | − | CAACCCCUACCUCGCCCCAG | 20 | 4716 |
| HSV1-UL30-16 | − | AACCCCUACCUCGCCCCAGU | 20 | 3244 |
| HSV1-UL30-251 | − | UCGCCCCAGUCGGGACGCAA | 20 | 4717 |
| HSV1-UL30-252 | − | CGGGACGCAACAGAAGCCGA | 20 | 4718 |
| HSV1-UL30-253 | − | CCAGCGCCAUACGUACUAUA | 20 | 4719 |
| HSV1-UL30-254 | − | UACGUACUAUAGCGAAUGCG | 20 | 4720 |
| HSV1-UL30-255 | − | GAAUUUCGAUUCAUCGCCCC | 20 | 4721 |
| HSV1-UL30-256 | − | GAUUCAUCGCCCCGCGGGUG | 20 | 4722 |
| HSV1-UL30-257 | − | CAUCGCCCCGCGGGUGCUGG | 20 | 4723 |
| HSV1-UL30-258 | − | UCGCCCCGCGGGUGCUGGAC | 20 | 4724 |
| HSV1-UL30-259 | − | UGCUGGACGAGGAUGCCCCC | 20 | 4725 |
| HSV1-UL30-24 | − | GCUGGACGAGGAUGCCCCCC | 20 | 3247 |
| HSV1-UL30-261 | − | UGGACGAGGAUGCCCCCCCG | 20 | 4726 |
| HSV1-UL30-262 | − | UGCCCCCCCGGAGAAGCGCG | 20 | 4727 |
| HSV1-UL30-25 | − | GCCCCCCCGGAGAAGCGCGC | 20 | 3131 |
| HSV1-UL30-264 | − | GCGCGCCCCCAAGGUGUACU | 20 | 4728 |
| HSV1-UL30-30 | − | CGCGCCCCCAAGGUGUACUG | 20 | 3135 |
| HSV1-UL30-31 | − | GCGCCCCCAAGGUGUACUGC | 20 | 3136 |
| HSV1-UL30-32 | − | CGCCCCCAAGGUGUACUGCG | 20 | 3137 |
| HSV1-UL30-33 | − | GCCCCCAAGGUGUACUGCGG | 20 | 3138 |
| HSV1-UL30-34 | − | CCCCCAAGGUGUACUGCGGG | 20 | 3249 |
| HSV1-UL30-270 | − | CAAGGUGUACUGCGGGGGGG | 20 | 4729 |
| HSV1-UL30-271 | − | CGAGCGCGACGUCCUCCGCG | 20 | 4730 |
| HSV1-UL30-272 | − | GCGACGUCCUCCGCGUCGGG | 20 | 4731 |
| HSV1-UL30-273 | − | UGGCCGCGGCGCUCGCGCCU | 20 | 4732 |
| HSV1-UL30-43 | − | GGCCGCGGCGCUCGCGCCUG | 20 | 3253 |
| HSV1-UL30-275 | − | GCUCGCGCCUGUGGGGCGGC | 20 | 4733 |
| HSV1-UL30-276 | − | GCGGCGUGGACCACGCCCCG | 20 | 4734 |
| HSV1-UL30-49 | − | CGGCGUGGACCACGCCCCGG | 20 | 3259 |
| HSV1-UL30-278 | − | UCUUUCACGUGUACGACAUC | 20 | 4735 |
| HSV1-UL30-52 | − | CUUUCACGUGUACGACAUCC | 20 | 3146 |
| HSV1-UL30-280 | − | UUCACGUGUACGACAUCCUG | 20 | 4736 |
| HSV1-UL30-281 | − | UGUACGACAUCCUGGAGAAC | 20 | 4737 |
| HSV1-UL30-53 | − | GUACGACAUCCUGGAGAACG | 20 | 3147 |
| HSV1-UL30-283 | − | CGGCGGCCCGCUGUCCC | 17 | 4738 |
| HSV1-UL30-119 | − | GGCGGCCCGCUGUCCCC | 17 | 3295 |
| HSV1-UL30-285 | − | CGGCCCGCUGUCCCCCG | 17 | 4739 |
| HSV1-UL30-120 | − | GGCCCGCUGUCCCCCGG | 17 | 3296 |
| HSV1-UL30-287 | − | GGAGGAAAGUCGGCGGC | 17 | 4740 |
| HSV1-UL30-288 | − | GGCGGCCAGGGCGGCGU | 17 | 4741 |
| HSV1-UL30-289 | − | UGCGCCCGCCGGCCCUC | 17 | 4742 |
| HSV1-UL30-129 | − | GCGCCCGCCGGCCCUCG | 17 | 3302 |
| HSV1-UL30-291 | − | GGCCCUCGCGGAGCCAG | 17 | 4743 |
| HSV1-UL30-130 | − | GCCCUCGCGGAGCCAGC | 17 | 3303 |
| HSV1-UL30-131 | − | CCCUCGCGGAGCCAGCC | 17 | 3304 |
| HSV1-UL30-294 | − | GGGGACCCCCGCCUUGU | 17 | 4744 |
| HSV1-UL30-295 | − | CCCCUACCUCGCCCCAG | 17 | 4745 |
| HSV1-UL30-134 | − | CCCUACCUCGCCCCAGU | 17 | 3307 |
| HSV1-UL30-297 | − | CCCCAGUCGGGACGCAA | 17 | 4746 |
| HSV1-UL30-298 | − | GACGCAACAGAAGCCGA | 17 | 4747 |
| HSV1-UL30-299 | − | GCGCCAUACGUACUAUA | 17 | 4748 |
| HSV1-UL30-300 | − | GUACUAUAGCGAAUGCG | 17 | 4749 |
| HSV1-UL30-301 | − | UUUCGAUUCAUCGCCCC | 17 | 4750 |
| HSV1-UL30-302 | − | UCAUCGCCCCGCGGGUG | 17 | 4751 |
| HSV1-UL30-303 | − | CGCCCCGCGGGUGCUGG | 17 | 4752 |
| HSV1-UL30-304 | − | CCCCGCGGGUGCUGGAC | 17 | 4753 |
| HSV1-UL30-305 | − | UGGACGAGGAUGCCCCC | 17 | 4754 |
| HSV1-UL30-142 | − | GGACGAGGAUGCCCCCC | 17 | 3310 |
| HSV1-UL30-307 | − | ACGAGGAUGCCCCCCCG | 17 | 4755 |
| HSV1-UL30-308 | − | CCCCCCGGAGAAGCGCG | 17 | 4756 |
| HSV1-UL30-143 | − | CCCCCGGAGAAGCGCGC | 17 | 3311 |
| HSV1-UL30-310 | − | CGCCCCCAAGGUGUACU | 17 | 4757 |
| HSV1-UL30-148 | − | GCCCCCAAGGUGUACUG | 17 | 3189 |
| HSV1-UL30-149 | − | CCCCCAAGGUGUACUGC | 17 | 3313 |
| HSV1-UL30-150 | − | CCCCAAGGUGUACUGCG | 17 | 3190 |
| HSV1-UL30-151 | − | CCCAAGGUGUACUGCGG | 17 | 3314 |
| HSV1-UL30-152 | − | CCAAGGUGUACUGCGGG | 17 | 3315 |
| HSV1-UL30-316 | − | GGUGUACUGCGGGGGGG | 17 | 4758 |
| HSV1-UL30-317 | − | GCGCGACGUCCUCCGCG | 17 | 4759 |
| HSV1-UL30-318 | − | ACGUCCUCCGCGUCGGG | 17 | 4760 |
| HSV1-UL30-319 | − | CCGCGGCGCUCGCGCCU | 17 | 4761 |
| HSV1-UL30-161 | − | CGCGGCGCUCGCGCCUG | 17 | 3320 |
| HSV1-UL30-321 | − | CGCGCCUGUGGGGCGGC | 17 | 4762 |
| HSV1-UL30-322 | − | GCGUGGACCACGCCCCG | 17 | 4763 |
| HSV1-UL30-167 | − | CGUGGACCACGCCCCGG | 17 | 3197 |
| HSV1-UL30-324 | − | UUCACGUGUACGACAUC | 17 | 4764 |
| HSV1-UL30-170 | − | UCACGUGUACGACAUCC | 17 | 3200 |
| HSV1-UL30-326 | − | ACGUGUACGACAUCCUG | 17 | 4765 |
| HSV1-UL30-327 | − | ACGACAUCCUGGAGAAC | 17 | 4766 |
| HSV1-UL30-171 | − | CGACAUCCUGGAGAACG | 17 | 3201 |
| HSV1-UL30-329 | + | UACGCGUGCUCCACGUUCUC | 20 | 4767 |
| HSV1-UL30-330 | + | UUCUCCAGGAUGUCGUACAC | 20 | 4768 |
| HSV1-UL30-331 | + | ACACGUGAAAGACGGUGACG | 20 | 4769 |
| HSV1-UL30-59 | + | CACGUGAAAGACGGUGACGG | 20 | 3262 |
| HSV1-UL30-333 | + | GAAAGACGGUGACGGUGGGG | 20 | 4770 |
| HSV1-UL30-334 | + | GACGGUGGGGUUGAACCCCG | 20 | 4771 |
| HSV1-UL30-62 | + | ACGGUGGGGUUGAACCCCGC | 20 | 3152 |
| HSV1-UL30-336 | + | GUCCACGCCGCCCCACAGGC | 20 | 4772 |
| HSV1-UL30-337 | + | ACAGGCGCGAGCGCCGCGGC | 20 | 4773 |
| HSV1-UL30-338 | + | CAGAAGCCGCCCGACCCGAC | 20 | 4774 |
| HSV1-UL30-68 | + | AGAAGCCGCCCGACCCGACG | 20 | 3155 |
| HSV1-UL30-340 | + | AAGCCGCCCGACCCGACGCG | 20 | 4775 |
| HSV1-UL30-341 | + | CGUCCCCCCCGCAGUACACC | 20 | 4776 |
| HSV1-UL30-70 | + | GUCCCCCCCGCAGUACACCU | 20 | 3157 |
| HSV1-UL30-71 | + | UCCCCCCCGCAGUACACCUU | 20 | 3267 |
| HSV1-UL30-344 | + | AGUACACCUUGGGGGCGCGC | 20 | 4777 |
| HSV1-UL30-345 | + | GUGCACCCCGGCGCGCUUCU | 20 | 4778 |
| HSV1-UL30-76 | + | UGCACCCCGGCGCGCUUCUC | 20 | 3270 |
| HSV1-UL30-77 | + | GCACCCCGGCGCGCUUCUCC | 20 | 3271 |
| HSV1-UL30-78 | + | CACCCCGGCGCGCUUCUCCG | 20 | 3272 |
| HSV1-UL30-79 | + | ACCCCGGCGCGCUUCUCCGG | 20 | 3273 |
| HSV1-UL30-350 | + | GGCAUCCUCGUCCAGCACCC | 20 | 4779 |
| HSV1-UL30-82 | + | GCAUCCUCGUCCAGCACCCG | 20 | 3161 |
| HSV1-UL30-352 | + | CGUCCAGCACCCGCGGGGCG | 20 | 4780 |
| HSV1-UL30-353 | + | AGCACCCGCGGGGCGAUGAA | 20 | 4781 |
| HSV1-UL30-354 | + | UCGCUAUAGUACGUAUGGCG | 20 | 4782 |
| HSV1-UL30-355 | + | CGGCUUCUGUUGCGUCCCGA | 20 | 4783 |
| HSV1-UL30-91 | + | GGCUUCUGUUGCGUCCCGAC | 20 | 3166 |
| HSV1-UL30-357 | + | UCUGUUGCGUCCCGACUGGG | 20 | 4784 |
| HSV1-UL30-358 | + | GCGUCCCGACUGGGGCGAGG | 20 | 4785 |
| HSV1-UL30-95 | + | CGUCCCGACUGGGGCGAGGU | 20 | 3170 |
| HSV1-UL30-360 | + | AAAGUUUUGCCUCAAACAAG | 20 | 4786 |
| HSV1-UL30-99 | + | AAGUUUUGCCUCAAACAAGG | 20 | 3282 |
| HSV1-UL30-100 | + | AGUUUUGCCUCAAACAAGGC | 20 | 3283 |
| HSV1-UL30-363 | + | GGGGGUCCCCGGCUGGCUCC | 20 | 4787 |
| HSV1-UL30-364 | + | GGGUCCCCGGCUGGCUCCGC | 20 | 4788 |
| HSV1-UL30-365 | + | GGCUGGCUCCGCGAGGGCCG | 20 | 4789 |
| HSV1-UL30-366 | + | GGCCGGCGGGCGCAAAAAAC | 20 | 4790 |
| HSV1-UL30-367 | + | CCCUGGCCGCCGACUUUCCU | 20 | 4791 |
| HSV1-UL30-112 | + | CCUGGCCGCCGACUUUCCUC | 20 | 3290 |
| HSV1-UL30-113 | + | CUGGCCGCCGACUUUCCUCC | 20 | 3291 |
| HSV1-UL30-114 | + | UGGCCGCCGACUUUCCUCCG | 20 | 3176 |
| HSV1-UL30-371 | + | CGACUUUCCUCCGGGGGACA | 20 | 4792 |
| HSV1-UL30-372 | + | GGGACAGCGGGCCGCCGCCA | 20 | 4793 |
| HSV1-UL30-118 | + | GGACAGCGGGCCGCCGCCAC | 20 | 3294 |
| HSV1-UL30-374 | + | GCGUGCUCCACGUUCUC | 17 | 4794 |
| HSV1-UL30-375 | + | UCCAGGAUGUCGUACAC | 17 | 4795 |
| HSV1-UL30-376 | + | CGUGAAAGACGGUGACG | 17 | 4796 |
| HSV1-UL30-177 | + | GUGAAAGACGGUGACGG | 17 | 3327 |
| HSV1-UL30-378 | + | AGACGGUGACGGUGGGG | 17 | 4797 |
| HSV1-UL30-379 | + | GGUGGGGUUGAACCCCG | 17 | 4798 |
| HSV1-UL30-180 | + | GUGGGGUUGAACCCCGC | 17 | 3329 |
| HSV1-UL30-381 | + | CACGCCGCCCCACAGGC | 17 | 4799 |
| HSV1-UL30-382 | + | GGCGCGAGCGCCGCGGC | 17 | 4800 |
| HSV1-UL30-383 | + | AAGCCGCCCGACCCGAC | 17 | 4801 |
| HSV1-UL30-186 | + | AGCCGCCCGACCCGACG | 17 | 3207 |
| HSV1-UL30-385 | + | CCGCCCGACCCGACGCG | 17 | 4802 |
| HSV1-UL30-386 | + | CCCCCCCGCAGUACACC | 17 | 4803 |
| HSV1-UL30-188 | + | CCCCCCGCAGUACACCU | 17 | 3209 |
| HSV1-UL30-189 | + | CCCCCGCAGUACACCUU | 17 | 3210 |
| HSV1-UL30-389 | + | ACACCUUGGGGGCGCGC | 17 | 4804 |
| HSV1-UL30-390 | + | CACCCCGGCGCGCUUCU | 17 | 4805 |
| HSV1-UL30-194 | + | ACCCCGGCGCGCUUCUC | 17 | 3335 |
| HSV1-UL30-195 | + | CCCCGGCGCGCUUCUCC | 17 | 3336 |
| HSV1-UL30-196 | + | CCCGGCGCGCUUCUCCG | 17 | 3213 |
| HSV1-UL30-197 | + | CCGGCGCGCUUCUCCGG | 17 | 3214 |
| HSV1-UL30-395 | + | AUCCUCGUCCAGCACCC | 17 | 4806 |
| HSV1-UL30-200 | + | UCCUCGUCCAGCACCCG | 17 | 3337 |
| HSV1-UL30-397 | + | CCAGCACCCGCGGGGCG | 17 | 4807 |
| HSV1-UL30-398 | + | ACCCGCGGGGCGAUGAA | 17 | 4808 |
| HSV1-UL30-399 | + | CUAUAGUACGUAUGGCG | 17 | 4809 |
| HSV1-UL30-400 | + | CUUCUGUUGCGUCCCGA | 17 | 4810 |
| HSV1-UL30-209 | + | UUCUGUUGCGUCCCGAC | 17 | 3222 |
| HSV1-UL30-402 | + | GUUGCGUCCCGACUGGG | 17 | 4811 |
| HSV1-UL30-403 | + | UCCCGACUGGGGCGAGG | 17 | 4812 |
| HSV1-UL30-213 | + | CCCGACUGGGGCGAGGU | 17 | 3341 |
| HSV1-UL30-405 | + | GUUUUGCCUCAAACAAG | 17 | 4813 |
| HSV1-UL30-217 | + | UUUUGCCUCAAACAAGG | 17 | 3344 |
| HSV1-UL30-218 | + | UUUGCCUCAAACAAGGC | 17 | 3345 |
| HSV1-UL30-408 | + | GGUCCCCGGCUGGCUCC | 17 | 4814 |
| HSV1-UL30-409 | + | UCCCCGGCUGGCUCCGC | 17 | 4815 |
| HSV1-UL30-410 | + | UGGCUCCGCGAGGGCCG | 17 | 4816 |
| HSV1-UL30-411 | + | CGGCGGGCGCAAAAAAC | 17 | 4817 |
| HSV1-UL30-412 | + | UGGCCGCCGACUUUCCU | 17 | 4818 |
| HSV1-UL30-230 | + | GGCCGCCGACUUUCCUC | 17 | 3228 |
| HSV1-UL30-231 | + | GCCGCCGACUUUCCUCC | 17 | 3229 |
| HSV1-UL30-232 | + | CCGCCGACUUUCCUCCG | 17 | 3230 |
| HSV1-UL30-416 | + | CUUUCCUCCGGGGGACA | 17 | 4819 |
| HSV1-UL30-417 | + | ACAGCGGGCCGCCGCCA | 17 | 4820 |
| HSV1-UL30-236 | + | CAGCGGGCCGCCGCCAC | 17 | 3358 |

Table 2E provides exemplary targeting domains for knocking out the UL30 gene selected according to the second tier parameters. The targeting domains are selected based on location within the coding sequence (but downstream of the first 500 bp) of the UL30 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2E

| 2nd Tier | DNA | | Target Site | |
|---|---|---|---|---|
| gRNA Name | Strand | Targeting Domain | Length | Seq ID |
| HSV1-UL30-1771 | − | UUAUGGACGCCAUCACACCG | 20 | 4821 |
| HSV1-UL30-1772 | − | UAUGGACGCCAUCACACCGA | 20 | 4822 |
| HSV1-UL30-1773 | − | AUGGACGCCAUCACACCGAC | 20 | 4823 |
| HSV1-UL30-1774 | − | CGGGGACCGUCAUCACGCUC | 20 | 4824 |
| HSV1-UL30-1775 | − | UCACGCUCCUGGGCCUGACU | 20 | 4825 |
| HSV1-UL30-1776 | − | CACGCUCCUGGGCCUGACUC | 20 | 4826 |
| HSV1-UL30-1777 | − | GGCCUGACUCCGGAAGGCCA | 20 | 4827 |
| HSV1-UL30-1778 | − | GCACGCGGCAGUACUUUUAC | 20 | 4828 |
| HSV1-UL30-1779 | − | GGCAGUACUUUUACAUGAAC | 20 | 4829 |
| HSV1-UL30-1780 | − | GCAGUACUUUUACAUGAACA | 20 | 4830 |
| HSV1-UL30-1781 | − | AGUACUUUUACAUGAACAAG | 20 | 4831 |
| HSV1-UL30-1782 | − | GUACUUUUACAUGAACAAGG | 20 | 4832 |
| HSV1-UL30-1783 | − | CACCUACAAUGCCGCGCCCC | 20 | 4833 |
| HSV1-UL30-1784 | − | CCGCGCCCCACGAGAUCUCU | 20 | 4834 |
| HSV1-UL30-1785 | − | GCGCAUGGCCGCGGCCCUGC | 20 | 4835 |
| HSV1-UL30-1786 | − | CCGCGGCCCUGCGCGAGUCC | 20 | 4836 |
| HSV1-UL30-1787 | − | CGUCGUUCCGCGGCAUCUCC | 20 | 4837 |
| HSV1-UL30-1788 | − | CGGCAUCUCCGCGGACCACU | 20 | 4838 |
| HSV1-UL30-1789 | − | UCUCCGCGGACCACUUCGAG | 20 | 4839 |
| HSV1-UL30-1790 | − | CUCCGCGGACCACUUCGAGG | 20 | 4840 |
| HSV1-UL30-1791 | − | ACCACUUCGAGGCGGAGGUG | 20 | 4841 |
| HSV1-UL30-1792 | − | CCACUUCGAGGCGGAGGUGG | 20 | 4842 |
| HSV1-UL30-1793 | − | GCGCACCGACGUGUACUACU | 20 | 4843 |
| HSV1-UL30-1794 | − | CUGUUUUACCGCGUCUACGU | 20 | 4844 |
| HSV1-UL30-1795 | − | UUACCGCGUCUACGUCCGAA | 20 | 4845 |
| HSV1-UL30-1796 | − | ACAACUUCUGCCCGGCCAUC | 20 | 4846 |
| HSV1-UL30-1797 | − | CUGCCCGGCCAUCAAGAAGU | 20 | 4847 |
| HSV1-UL30-1798 | − | GCCCGGCCAUCAAGAAGUAC | 20 | 4848 |
| HSV1-UL30-1799 | − | GGCCAUCAAGAAGUACGAGG | 20 | 4849 |
| HSV1-UL30-1800 | − | GCCAUCAAGAAGUACGAGGG | 20 | 4850 |
| HSV1-UL30-1801 | − | ACGCCACCACCCGGUUCAUC | 20 | 4851 |
| HSV1-UL30-1802 | − | CCGGUUCAUCCUGGACAACC | 20 | 4852 |
| HSV1-UL30-1803 | − | UCGGCUGGUACCGUCUCAAA | 20 | 4853 |
| HSV1-UL30-1804 | − | UGGUACCGUCUCAAACCGGG | 20 | 4854 |
| HSV1-UL30-1805 | − | GGUACCGUCUCAAACCGGGC | 20 | 4855 |
| HSV1-UL30-1806 | − | GCCGGCGGCCCCGAUGGCCU | 20 | 4856 |
| HSV1-UL30-1807 | − | CCGGCGGCCCCGAUGGCCUU | 20 | 4857 |
| HSV1-UL30-1808 | − | CUUCGGGACAUCCAGCGACG | 20 | 4858 |
| HSV1-UL30-1809 | − | ACGUCGAGUUUAACUGUACG | 20 | 4859 |
| HSV1-UL30-1810 | − | UACGGCGGACAACCUGGCCA | 20 | 4860 |
| HSV1-UL30-1811 | − | CGGCGGACAACCUGGCCAUC | 20 | 4861 |
| HSV1-UL30-1812 | − | GGCGGACAACCUGGCCAUCG | 20 | 4862 |
| HSV1-UL30-1813 | − | GCGGACAACCUGGCCAUCGA | 20 | 4863 |
| HSV1-UL30-1814 | − | CGGACAACCUGGCCAUCGAG | 20 | 4864 |
| HSV1-UL30-1815 | − | ACCUGGCCAUCGAGGGGGGC | 20 | 4865 |
| HSV1-UL30-1816 | − | CAAGCUCAUGUGCUUCGAUA | 20 | 4866 |
| HSV1-UL30-1817 | − | GCUUCGAUAUCGAAUGCAAG | 20 | 4867 |
| HSV1-UL30-1818 | − | CUUCGAUAUCGAAUGCAAGG | 20 | 4868 |
| HSV1-UL30-1819 | − | UUCGAUAUCGAAUGCAAGGC | 20 | 4869 |
| HSV1-UL30-1820 | − | UCGAUAUCGAAUGCAAGGCG | 20 | 4870 |
| HSV1-UL30-1821 | − | CGAUAUCGAAUGCAAGGCGG | 20 | 4871 |
| HSV1-UL30-1822 | − | GAUAUCGAAUGCAAGGCGGG | 20 | 4872 |
| HSV1-UL30-1823 | − | AUAUCGAAUGCAAGGCGGGG | 20 | 4873 |
| HSV1-UL30-1824 | − | UAUCGAAUGCAAGGCGGGGG | 20 | 4874 |
| HSV1-UL30-1825 | − | UCGAAUGCAAGGCGGGGGGG | 20 | 4875 |
| HSV1-UL30-1826 | − | AUGCAAGGCGGGGGGGGAGG | 20 | 4876 |
| HSV1-UL30-1827 | − | CGAGCUGGCCUUUCCGGUGG | 20 | 4877 |
| HSV1-UL30-1828 | − | CCUUUCCGGUGGCCGGGCAC | 20 | 4878 |
| HSV1-UL30-1829 | − | CUUUCCGGUGGCCGGGCACC | 20 | 4879 |
| HSV1-UL30-1830 | − | UUCCGGUGGCCGGGCACCCG | 20 | 4880 |
| HSV1-UL30-1831 | − | ACGACCUGUCCACCACCGCC | 20 | 4881 |
| HSV1-UL30-1832 | − | CGACCUGUCCACCACCGCCC | 20 | 4882 |
| HSV1-UL30-1833 | − | GCUCGGUUCCUGCGACCUCC | 20 | 4883 |
| HSV1-UL30-1834 | − | GCGACCUCCCCGAAUCCCAC | 20 | 4884 |
| HSV1-UL30-1835 | − | CCUCCCCGAAUCCCACCUGA | 20 | 4885 |
| HSV1-UL30-1836 | − | CACCUGAACGAGCUGGCGGC | 20 | 4886 |
| HSV1-UL30-1837 | − | ACCUGAACGAGCUGGCGGCC | 20 | 4887 |
| HSV1-UL30-1838 | − | GCCUGCCCACGCCCGUGGUU | 20 | 4888 |
| HSV1-UL30-1839 | − | CCUGCCCACGCCCGUGGUUC | 20 | 4889 |
| HSV1-UL30-1840 | − | CGUGGUUCUGGAAUUCGACA | 20 | 4890 |
| HSV1-UL30-1841 | − | UCUGGAAUUCGACAGCGAAU | 20 | 4891 |
| HSV1-UL30-1842 | − | UGUUGGCCUUCAUGACCCUU | 20 | 4892 |
| HSV1-UL30-1843 | − | CCUUGUGAAACAGUACGGCC | 20 | 4893 |
| HSV1-UL30-1844 | − | GUACGGCCCCGAGUUCGUGA | 20 | 4894 |
| HSV1-UL30-1845 | − | CCUUCUUGCUGGCCAAGCUG | 20 | 4895 |
| HSV1-UL30-1846 | − | CGGACAUUUACAAGGUCCCC | 20 | 4896 |
| HSV1-UL30-1847 | − | CAUUUACAAGGUCCCCCUGG | 20 | 4897 |
| HSV1-UL30-1848 | − | CCCUGGACGGGUACGGCCGC | 20 | 4898 |
| HSV1-UL30-1849 | − | GGGUACGGCCGCAUGAACGG | 20 | 4899 |
| HSV1-UL30-1850 | − | GGUACGGCCGCAUGAACGGC | 20 | 4900 |
| HSV1-UL30-1851 | − | GGCCGGGGCGUGUUUCGCGU | 20 | 4901 |
| HSV1-UL30-1852 | − | GCCGGGGCGUGUUUCGCGUG | 20 | 4902 |
| HSV1-UL30-1853 | − | UUCGCGUGUGGGACAUAGGC | 20 | 4903 |
| HSV1-UL30-1854 | − | ACAUAGGCCAGAGCCACUUC | 20 | 4904 |
| HSV1-UL30-1855 | − | AGAAGCGCAGCAAGAUAAAG | 20 | 4905 |
| HSV1-UL30-1856 | − | AGAUAAAGGUGAACGGCAUG | 20 | 4906 |
| HSV1-UL30-1857 | − | CAUGGUGAACAUCGACAUGU | 20 | 4907 |
| HSV1-UL30-1858 | − | AUGGUGAACAUCGACAUGUA | 20 | 4908 |
| HSV1-UL30-1859 | − | UAACCGACAAGAUCAAGCUC | 20 | 4909 |
| HSV1-UL30-1860 | − | CUACAAGCUCAACGCCGUGG | 20 | 4910 |
| HSV1-UL30-1861 | − | ACGCCGUGGCCGAAGCCGUC | 20 | 4911 |
| HSV1-UL30-1862 | − | CCGUGGCCGAAGCCGUCCUG | 20 | 4912 |
| HSV1-UL30-1863 | − | CCGAAGCCGUCCUGAAGGAC | 20 | 4913 |
| HSV1-UL30-1864 | − | AAGCCGUCCUGAAGGACAAG | 20 | 4914 |
| HSV1-UL30-1865 | − | CCGUCCUGAAGGACAAGAAG | 20 | 4915 |
| HSV1-UL30-1866 | − | UGAAGGACAAGAAGAAGGAC | 20 | 4916 |
| HSV1-UL30-1867 | − | CAUCCCCGCCUACUACGCCG | 20 | 4917 |
| HSV1-UL30-1868 | − | CGCCGCCGGGCCCGCGCAAC | 20 | 4918 |
| HSV1-UL30-534 | − | GCCGCCGGGCCCGCGCAACG | 20 | 3472 |
| HSV1-UL30-1870 | − | CGCGCAACGCGGGGUGAUCG | 20 | 4919 |
| HSV1-UL30-1871 | − | UGAUCGGCGAGUACUGCAUA | 20 | 4920 |
| HSV1-UL30-1872 | − | GCAUACAGGAUUCCCUGCUG | 20 | 4921 |
| HSV1-UL30-1873 | − | UUUUUAAGUUUUUGCCCCAU | 20 | 4922 |
| HSV1-UL30-1874 | − | UUUUAAGUUUUUGCCCCAUC | 20 | 4923 |
| HSV1-UL30-1875 | − | UCUCGGCCGUCGCGCGCUUG | 20 | 4924 |
| HSV1-UL30-1876 | − | GCCUGCUGCGCCUGGCCGAC | 20 | 4925 |
| HSV1-UL30-1877 | − | UGCUGCGCCUGGCCGACCAG | 20 | 4926 |
| HSV1-UL30-1878 | − | ACCAGAAGGGCUUUAUUCUG | 20 | 4927 |
| HSV1-UL30-1879 | − | GCUUUAUUCUGCCGGACACC | 20 | 4928 |
| HSV1-UL30-1880 | − | CUUUAUUCUGCCGGACACCC | 20 | 4929 |
| HSV1-UL30-1881 | − | CCGGACACCCAGGGGCGAUU | 20 | 4930 |
| HSV1-UL30-1882 | − | CGGACACCCAGGGGCGAUUU | 20 | 4931 |
| HSV1-UL30-1883 | − | CCAGGGGCGAUUUAGGGGCG | 20 | 4932 |
| HSV1-UL30-1884 | − | CAGGGGCGAUUUAGGGGCGC | 20 | 4933 |
| HSV1-UL30-1885 | − | AGGGGCGAUUUAGGGGCGCC | 20 | 4934 |
| HSV1-UL30-1886 | − | GGGGCGAUUUAGGGGCGCCG | 20 | 4935 |
| HSV1-UL30-1887 | − | GGGCGAUUUAGGGGCGCCGG | 20 | 4936 |
| HSV1-UL30-1888 | − | GGCGAUUUAGGGGCGCCGGG | 20 | 4937 |
| HSV1-UL30-1889 | − | GCGAUUUAGGGGCGCCGGGG | 20 | 4938 |
| HSV1-UL30-1890 | − | CCCAAGCGUCCGGCCGCAGC | 20 | 4939 |
| HSV1-UL30-1891 | − | CCAAGCGUCCGGCCGCAGCC | 20 | 4940 |
| HSV1-UL30-1892 | − | CAAGCGUCCGGCCGCAGCCC | 20 | 4941 |
| HSV1-UL30-1893 | − | AGCGUCCGGCCGCAGCCCGG | 20 | 4942 |
| HSV1-UL30-1894 | − | UCCGGCCGCAGCCCGGGAGG | 20 | 4943 |
| HSV1-UL30-1895 | − | CGGCCGCAGCCCGGGAGGAC | 20 | 4944 |
| HSV1-UL30-569 | − | GGCCGCAGCCCGGGAGGACG | 20 | 3507 |
| HSV1-UL30-1897 | − | CCGGGAGGACGAGGAGCGGC | 20 | 4945 |
| HSV1-UL30-1898 | − | GGGAGGACGAGGAGCGGCCA | 20 | 4946 |
| HSV1-UL30-571 | − | GGAGGACGAGGAGCGGCCAG | 20 | 3509 |
| HSV1-UL30-1900 | − | AGGACGAGGAGCGGCCAGAG | 20 | 4947 |
| HSV1-UL30-572 | − | GGACGAGGAGCGGCCAGAGG | 20 | 3510 |
| HSV1-UL30-1902 | − | ACGAGGAGCGGCCAGAGGAG | 20 | 4948 |
| HSV1-UL30-573 | − | CGAGGAGCGGCCAGAGGAGG | 20 | 3511 |
| HSV1-UL30-574 | − | GAGGAGCGGCCAGAGGAGGA | 20 | 3512 |
| HSV1-UL30-575 | − | AGGAGCGGCCAGAGGAGGAG | 20 | 3513 |
| HSV1-UL30-576 | − | GGAGCGGCCAGAGGAGGAGG | 20 | 3514 |
| HSV1-UL30-1907 | − | AGCGGCCAGAGGAGGAGGGG | 20 | 4949 |
| HSV1-UL30-1908 | − | GCCAGAGGAGGAGGGGGAGG | 20 | 4950 |
| HSV1-UL30-1909 | − | CAGAGGAGGAGGGGGAGGAC | 20 | 4951 |
| HSV1-UL30-1910 | − | GGAGGAGGGGGAGGACGAGG | 20 | 4952 |
| HSV1-UL30-1911 | − | GGGGGAGGACGAGGACGAAC | 20 | 4953 |
| HSV1-UL30-1912 | − | GGGAGGACGAGGACGAACGC | 20 | 4954 |
| HSV1-UL30-579 | − | GGAGGACGAGGACGAACGCG | 20 | 3517 |
| HSV1-UL30-1914 | − | AGGACGAGGACGAACGCGAG | 20 | 4955 |
| HSV1-UL30-1915 | − | CGAGGACGAACGCGAGGAGG | 20 | 4956 |
| HSV1-UL30-582 | − | GAGGACGAACGCGAGGAGGG | 20 | 3520 |
| HSV1-UL30-583 | − | AGGACGAACGCGAGGAGGGC | 20 | 3521 |
| HSV1-UL30-1918 | − | CGAACGCGAGGAGGGCGGGG | 20 | 4957 |
| HSV1-UL30-1919 | − | CGCGAGGAGGGCGGGGGCGA | 20 | 4958 |
| HSV1-UL30-586 | − | GCGAGGAGGGCGGGGGCGAG | 20 | 3524 |
| HSV1-UL30-587 | − | CGAGGAGGGCGGGGGCGAGC | 20 | 3525 |
| HSV1-UL30-1922 | − | AGGGCGGGGGCGAGCGGGAG | 20 | 4959 |
| HSV1-UL30-588 | − | GGGCGGGGGCGAGCGGGAGC | 20 | 3526 |
| HSV1-UL30-1924 | − | GCGGGGGCGAGCGGGAGCCG | 20 | 4960 |
| HSV1-UL30-1925 | − | GAGCGGGAGCCGGAGGGCGC | 20 | 4961 |
| HSV1-UL30-591 | − | AGCGGGAGCCGGAGGGCGCG | 20 | 3529 |
| HSV1-UL30-592 | − | GCGGGAGCCGGAGGGCGCGC | 20 | 3530 |
| HSV1-UL30-1928 | − | GGGAGACCGCCGGCAGGCAC | 20 | 4962 |
| HSV1-UL30-595 | − | GGAGACCGCCGGCAGGCACG | 20 | 3533 |
| HSV1-UL30-1930 | − | CCGGCAGGCACGUGGGGUAC | 20 | 4963 |
| HSV1-UL30-1931 | − | CGGCAGGCACGUGGGGUACC | 20 | 4964 |
| HSV1-UL30-1932 | − | GGCAGGCACGUGGGGUACCA | 20 | 4965 |
| HSV1-UL30-1933 | − | CACGUGGGGUACCAGGGGGC | 20 | 4966 |
| HSV1-UL30-1934 | − | CAGGGUCCUUGACCCCACUU | 20 | 4967 |
| HSV1-UL30-1935 | − | ACCCCACUUCCGGGUUUCAC | 20 | 4968 |
| HSV1-UL30-1936 | − | UGUGCUUCAGCACGCUCUCC | 20 | 4969 |
| HSV1-UL30-1937 | − | UGCUUCAGCACGCUCUCCCU | 20 | 4970 |
| HSV1-UL30-1938 | − | GGGCCGACGCAGUGGCGCAC | 20 | 4971 |
| HSV1-UL30-1939 | − | GGCCGACGCAGUGGCGCACC | 20 | 4972 |
| HSV1-UL30-1940 | − | ACGCAGUGGCGCACCUGGAG | 20 | 4973 |
| HSV1-UL30-1941 | − | UGGCGCACCUGGAGGCGGGC | 20 | 4974 |
| HSV1-UL30-1942 | − | UGGAGGCGGGCAAGGACUAC | 20 | 4975 |
| HSV1-UL30-1943 | − | GGAGGCGGGCAAGGACUACC | 20 | 4976 |
| HSV1-UL30-1944 | − | GGGCAAGGACUACCUGGAGA | 20 | 4977 |
| HSV1-UL30-1945 | − | AGGACUACCUGGAGAUCGAG | 20 | 4978 |
| HSV1-UL30-1946 | − | GGACUACCUGGAGAUCGAGG | 20 | 4979 |
| HSV1-UL30-1947 | − | GACUACCUGGAGAUCGAGGU | 20 | 4980 |
| HSV1-UL30-1948 | − | ACUACCUGGAGAUCGAGGUG | 20 | 4981 |
| HSV1-UL30-1949 | − | CUACCUGGAGAUCGAGGUGG | 20 | 4982 |
| HSV1-UL30-1950 | − | UUCUUCGUCAAGGCUCACGU | 20 | 4983 |
| HSV1-UL30-1951 | − | CUUCGUCAAGGCUCACGUGC | 20 | 4984 |
| HSV1-UL30-1952 | − | UCGUCAAGGCUCACGUGCGA | 20 | 4985 |
| HSV1-UL30-1953 | − | AGCCUCCUCAGCAUCCUCCU | 20 | 4986 |
| HSV1-UL30-1954 | − | GCCUCCUCAGCAUCCUCCUG | 20 | 4987 |
| HSV1-UL30-1955 | − | CUGCGGGACUGGCUCGCCAU | 20 | 4988 |
| HSV1-UL30-1956 | − | AUGCGAAAGCAGAUCCGCUC | 20 | 4989 |
| HSV1-UL30-1957 | − | AGAUCCGCUCGCGGAUUCCC | 20 | 4990 |
| HSV1-UL30-1958 | − | GCGGAUUCCCCAGAGCAGCC | 20 | 4991 |
| HSV1-UL30-1959 | − | GGAUUCCCCAGAGCAGCCCC | 20 | 4992 |
| HSV1-UL30-1960 | − | GAUUCCCCAGAGCAGCCCCG | 20 | 4993 |
| HSV1-UL30-1961 | − | GCCCCGAGGAGGCCGUGCUC | 20 | 4994 |
| HSV1-UL30-1962 | − | GGUCGUGUGUAACUCGGUGU | 20 | 4995 |
| HSV1-UL30-1963 | − | GUAACUCGGUGUACGGGUUC | 20 | 4996 |
| HSV1-UL30-1964 | − | UAACUCGGUGUACGGGUUCA | 20 | 4997 |
| HSV1-UL30-1965 | − | AACUCGGUGUACGGGUUCAC | 20 | 4998 |
| HSV1-UL30-1966 | − | CGGGUUCACGGGAGUGCAGC | 20 | 4999 |
| HSV1-UL30-1967 | − | GACGGUGACGACCAUCGGCC | 20 | 5000 |
| HSV1-UL30-1968 | − | CGAGAUGCUGCUCGCGACCC | 20 | 5001 |
| HSV1-UL30-1969 | − | CGCGAGUACGUCCACGCGCG | 20 | 5002 |
| HSV1-UL30-1970 | − | CCACGCGCGCUGGGCGGCCU | 20 | 5003 |
| HSV1-UL30-1971 | − | AACAGCUCCUGGCCGAUUUC | 20 | 5004 |
| HSV1-UL30-1972 | − | ACAGCUCCUGGCCGAUUUCC | 20 | 5005 |
| HSV1-UL30-1973 | − | GGCGGCCGACAUGCGCGCCC | 20 | 5006 |
| HSV1-UL30-1974 | − | CUAUUCCAUGCGCAUCAUCU | 20 | 5007 |
| HSV1-UL30-1975 | − | UAUUCCAUGCGCAUCAUCUA | 20 | 5008 |
| HSV1-UL30-1976 | − | AUUCCAUGCGCAUCAUCUAC | 20 | 5009 |
| HSV1-UL30-1977 | − | UGCGCAUCAUCUACGGGGAC | 20 | 5010 |
| HSV1-UL30-1978 | − | GUGCCGCGGCCUCACGGCCG | 20 | 5011 |
| HSV1-UL30-1979 | − | CGGCCGCCGGGCUGACGGCC | 20 | 5012 |
| HSV1-UL30-1980 | − | CGGCCGUGGGCGACAAGAUG | 20 | 5013 |
| HSV1-UL30-1981 | − | GUUUCUGCCCCCCAUCAAAC | 20 | 5014 |
| HSV1-UL30-1982 | − | GCCCCCCAUCAAACUCGAGU | 20 | 5015 |
| HSV1-UL30-1983 | − | CCAAGCUGCUGCUGAUCGCC | 20 | 5016 |
| HSV1-UL30-1984 | − | AAAGUACAUCGGCGUCAUCU | 20 | 5017 |
| HSV1-UL30-1985 | − | AAGUACAUCGGCGUCAUCUA | 20 | 5018 |
| HSV1-UL30-1986 | − | AGUACAUCGGCGUCAUCUAC | 20 | 5019 |
| HSV1-UL30-1987 | − | ACGGGGGUAAGAUGCUCAUC | 20 | 5020 |
| HSV1-UL30-1988 | − | GUAAGAUGCUCAUCAAGGGC | 20 | 5021 |
| HSV1-UL30-1989 | − | GCGUUUAUCAACCGCACCUC | 20 | 5022 |
| HSV1-UL30-1990 | − | GUUUUACGACGAUACCGUCU | 20 | 5023 |
| HSV1-UL30-1991 | − | UUUUACGACGAUACCGUCUC | 20 | 5024 |
| HSV1-UL30-1992 | − | CGGAGCGGCCGCCGCGUUAG | 20 | 5025 |
| HSV1-UL30-1993 | − | CCGCGUUAGCCGAGCGCCCC | 20 | 5026 |
| HSV1-UL30-1994 | − | CGCGUUAGCCGAGCGCCCCG | 20 | 5027 |
| HSV1-UL30-1995 | − | CGUUAGCCGAGCGCCCCGCG | 20 | 5028 |
| HSV1-UL30-1996 | − | GUUAGCCGAGCGCCCCGCGG | 20 | 5029 |
| HSV1-UL30-1997 | − | GUGGCUGGCGCGACCCCUGC | 20 | 5030 |
| HSV1-UL30-1998 | − | GGCUGGCGCGACCCCUGCCC | 20 | 5031 |
| HSV1-UL30-1999 | − | GCUGGCGCGACCCCUGCCCG | 20 | 5032 |
| HSV1-UL30-2000 | − | GCCCGAGGGACUGCAGGCGU | 20 | 5033 |
| HSV1-UL30-2001 | − | CCCGAGGGACUGCAGGCGUU | 20 | 5034 |
| HSV1-UL30-2002 | − | CCCAUCGGCGCAUCACCGAC | 20 | 5035 |
| HSV1-UL30-2003 | − | CCAUCGGCGCAUCACCGACC | 20 | 5036 |
| HSV1-UL30-2004 | − | AUCGGCGCAUCACCGACCCG | 20 | 5037 |
| HSV1-UL30-2005 | − | CGGCGCAUCACCGACCCGGA | 20 | 5038 |
| HSV1-UL30-2006 | − | GGCGCAUCACCGACCCGGAG | 20 | 5039 |
| HSV1-UL30-2007 | − | CCGACCCGGAGAGGGACAUC | 20 | 5040 |
| HSV1-UL30-2008 | − | CCAGGACUUUGUCCUCACCG | 20 | 5041 |
| HSV1-UL30-2009 | − | ACUUUGUCCUCACCGCCGAA | 20 | 5042 |
| HSV1-UL30-2010 | − | GCGCGCAGGUCCCGUCCAUC | 20 | 5043 |
| HSV1-UL30-2011 | − | CAGGUCCCGUCCAUCAAGGA | 20 | 5044 |
| HSV1-UL30-2012 | − | CGUGAUCGUGGCCCAGACCC | 20 | 5045 |
| HSV1-UL30-2013 | − | CGUGGCCCAGACCCGCGAGG | 20 | 5046 |
| HSV1-UL30-2014 | − | UGGCCCAGACCCGCGAGGUA | 20 | 5047 |
| HSV1-UL30-2015 | − | GGCCCAGACCCGCGAGGUAG | 20 | 5048 |
| HSV1-UL30-2016 | − | CGCGCGGCUGGCCGCCCUCC | 20 | 5049 |
| HSV1-UL30-2017 | − | CGAGCUAGACGCCGCCGCCC | 20 | 5050 |
| HSV1-UL30-2018 | − | GAGCUAGACGCCGCCGCCCC | 20 | 5051 |
| HSV1-UL30-2019 | − | AGCUAGACGCCGCCGCCCCA | 20 | 5052 |
| HSV1-UL30-2020 | − | AGACGCCGCCGCCCCAGGGG | 20 | 5053 |
| HSV1-UL30-2021 | − | CCCUCCCCGGCCAAGCGCCC | 20 | 5054 |
| HSV1-UL30-2022 | − | CCUCCCCGGCCAAGCGCCCC | 20 | 5055 |
| HSV1-UL30-2023 | − | CUCCCCGGCCAAGCGCCCCC | 20 | 5056 |
| HSV1-UL30-2024 | − | CGCCGUCGCCUGCCGACCCC | 20 | 5057 |
| HSV1-UL30-2025 | − | GCCGUCGCCUGCCGACCCCC | 20 | 5058 |
| HSV1-UL30-2026 | − | CCGUCGCCUGCCGACCCCCC | 20 | 5059 |
| HSV1-UL30-2027 | − | GCCCCGCAAGCUGCUGGUGU | 20 | 5060 |
| HSV1-UL30-2028 | − | GCUGCUGGUGUCCGAGCUGG | 20 | 5061 |
| HSV1-UL30-2029 | − | UGCUGGUGUCCGAGCUGGCC | 20 | 5062 |
| HSV1-UL30-2030 | − | CCAUUGCCCACGGCGUCGCC | 20 | 5063 |
| HSV1-UL30-2031 | − | CCCACGGCGUCGCCCUGAAC | 20 | 5064 |
| HSV1-UL30-2032 | − | ACUAUUACUUCUCCCACCUG | 20 | 5065 |
| HSV1-UL30-2033 | − | CUAUUACUUCUCCCACCUGU | 20 | 5066 |
| HSV1-UL30-2034 | − | UAUUACUUCUCCCACCUGUU | 20 | 5067 |
| HSV1-UL30-2035 | − | CGUGACAUUCAAGGCCCUGU | 20 | 5068 |
| HSV1-UL30-2036 | − | GUGACAUUCAAGGCCCUGUU | 20 | 5069 |
| HSV1-UL30-2037 | − | UGACAUUCAAGGCCCUGUUU | 20 | 5070 |
| HSV1-UL30-2038 | − | UGGGAAUAACGCCAAGAUCA | 20 | 5071 |
| HSV1-UL30-2039 | − | GGAAUAACGCCAAGAUCACC | 20 | 5072 |
| HSV1-UL30-2040 | − | UCUGUUAAAAAGGUUUAUUC | 20 | 5073 |
| HSV1-UL30-2041 | − | UUCCCGAAGUGUGGCACCCC | 20 | 5074 |
| HSV1-UL30-2042 | − | GACGACGUGGCCGCGCGGCU | 20 | 5075 |
| HSV1-UL30-2043 | − | GGCCGCGCGGCUCCGGACCG | 20 | 5076 |
| HSV1-UL30-2044 | − | GCGGCUCCGGACCGCAGGGU | 20 | 5077 |
| HSV1-UL30-2045 | − | CGGCUCCGGACCGCAGGGUU | 20 | 5078 |
| HSV1-UL30-2046 | − | GGACCGCAGGGUUCGGGGCG | 20 | 5079 |
| HSV1-UL30-2047 | − | CGGUGGGUGCCGGCGCUACG | 20 | 5080 |
| HSV1-UL30-2048 | − | GGUGGGUGCCGGCGCUACGG | 20 | 5081 |
| HSV1-UL30-2049 | − | UGGGUGCCGGCGCUACGGCG | 20 | 5082 |
| HSV1-UL30-2050 | − | GGGUGCCGGCGCUACGGCGG | 20 | 5083 |
| HSV1-UL30-2051 | − | GCUACGGCGGAGGAAACUCG | 20 | 5084 |
| HSV1-UL30-2052 | − | GAAACUCGUCGAAUGUUGCA | 20 | 5085 |
| HSV1-UL30-2053 | − | UGGACGCCAUCACACCG | 17 | 5086 |
| HSV1-UL30-2054 | − | GGACGCCAUCACACCGA | 17 | 5087 |
| HSV1-UL30-2055 | − | GACGCCAUCACACCGAC | 17 | 5088 |
| HSV1-UL30-2056 | − | GGACCGUCAUCACGCUC | 17 | 5089 |
| HSV1-UL30-2057 | − | CGCUCCUGGGCCUGACU | 17 | 5090 |
| HSV1-UL30-2058 | − | GCUCCUGGGCCUGACUC | 17 | 5091 |
| HSV1-UL30-2059 | − | CUGACUCCGGAAGGCCA | 17 | 5092 |
| HSV1-UL30-2060 | − | CGCGGCAGUACUUUUAC | 17 | 5093 |
| HSV1-UL30-2061 | − | AGUACUUUUACAUGAAC | 17 | 5094 |
| HSV1-UL30-2062 | − | GUACUUUUACAUGAACA | 17 | 5095 |
| HSV1-UL30-2063 | − | ACUUUUACAUGAACAAG | 17 | 5096 |
| HSV1-UL30-2064 | − | CUUUUACAUGAACAAGG | 17 | 5097 |
| HSV1-UL30-2065 | − | CUACAAUGCCGCGCCCC | 17 | 5098 |
| HSV1-UL30-2066 | − | CGCCCCACGAGAUCUCU | 17 | 5099 |
| HSV1-UL30-2067 | − | CAUGGCCGCGGCCCUGC | 17 | 5100 |
| HSV1-UL30-2068 | − | CGGCCCUGCGCGAGUCC | 17 | 5101 |
| HSV1-UL30-2069 | − | CGUUCCGCGGCAUCUCC | 17 | 5102 |
| HSV1-UL30-2070 | − | CAUCUCCGCGGACCACU | 17 | 5103 |
| HSV1-UL30-2071 | − | CCGCGGACCACUUCGAG | 17 | 5104 |
| HSV1-UL30-2072 | − | CGCGGACCACUUCGAGG | 17 | 5105 |
| HSV1-UL30-2073 | − | ACUUCGAGGCGGAGGUG | 17 | 5106 |
| HSV1-UL30-2074 | − | CUUCGAGGCGGAGGUGG | 17 | 5107 |
| HSV1-UL30-2075 | − | CACCGACGUGUACUACU | 17 | 5108 |
| HSV1-UL30-2076 | − | UUUUACCGCGUCUACGU | 17 | 5109 |
| HSV1-UL30-2077 | − | CCGCGUCUACGUCCGAA | 17 | 5110 |
| HSV1-UL30-2078 | − | ACUUCUGCCCGGCCAUC | 17 | 5111 |
| HSV1-UL30-2079 | − | CCCGGCCAUCAAGAAGU | 17 | 5112 |
| HSV1-UL30-2080 | − | CGGCCAUCAAGAAGUAC | 17 | 5113 |
| HSV1-UL30-2081 | − | CAUCAAGAAGUACGAGG | 17 | 5114 |
| HSV1-UL30-2082 | − | AUCAAGAAGUACGAGGG | 17 | 5115 |
| HSV1-UL30-2083 | − | CCACCACCCGGUUCAUC | 17 | 5116 |
| HSV1-UL30-2084 | − | GUUCAUCCUGGACAACC | 17 | 5117 |
| HSV1-UL30-2085 | − | GCUGGUACCGUCUCAAA | 17 | 5118 |
| HSV1-UL30-2086 | − | UACCGUCUCAAACCGGG | 17 | 5119 |
| HSV1-UL30-2087 | − | ACCGUCUCAAACCGGGC | 17 | 5120 |
| HSV1-UL30-2088 | − | GGCGGCCCCGAUGGCCU | 17 | 5121 |
| HSV1-UL30-2089 | − | GCGGCCCCGAUGGCCUU | 17 | 5122 |
| HSV1-UL30-2090 | − | CGGGACAUCCAGCGACG | 17 | 5123 |
| HSV1-UL30-2091 | − | UCGAGUUUAACUGUACG | 17 | 5124 |
| HSV1-UL30-2092 | − | GGCGGACAACCUGGCCA | 17 | 5125 |
| HSV1-UL30-2093 | − | CGGACAACCUGGCCAUC | 17 | 5126 |
| HSV1-UL30-2094 | − | GGACAACCUGGCCAUCG | 17 | 5127 |
| HSV1-UL30-2095 | − | GACAACCUGGCCAUCGA | 17 | 5128 |
| HSV1-UL30-2096 | − | ACAACCUGGCCAUCGAG | 17 | 5129 |
| HSV1-UL30-2097 | − | UGGCCAUCGAGGGGGGC | 17 | 5130 |
| HSV1-UL30-2098 | − | GCUCAUGUGCUUCGAUA | 17 | 5131 |
| HSV1-UL30-2099 | − | UCGAUAUCGAAUGCAAG | 17 | 5132 |
| HSV1-UL30-2100 | − | CGAUAUCGAAUGCAAGG | 17 | 5133 |
| HSV1-UL30-2101 | − | GAUAUCGAAUGCAAGGC | 17 | 5134 |
| HSV1-UL30-2102 | − | AUAUCGAAUGCAAGGCG | 17 | 5135 |
| HSV1-UL30-2103 | − | UAUCGAAUGCAAGGCGG | 17 | 5136 |
| HSV1-UL30-2104 | − | AUCGAAUGCAAGGCGGG | 17 | 5137 |
| HSV1-UL30-2105 | − | UCGAAUGCAAGGCGGGG | 17 | 5138 |
| HSV1-UL30-2106 | − | CGAAUGCAAGGCGGGGG | 17 | 5139 |
| HSV1-UL30-2107 | − | AAUGCAAGGCGGGGGGG | 17 | 5140 |
| HSV1-UL30-2108 | − | CAAGGCGGGGGGGGAGG | 17 | 5141 |
| HSV1-UL30-2109 | − | GCUGGCCUUUCCGGUGG | 17 | 5142 |
| HSV1-UL30-2110 | − | UUCCGGUGGCCGGGCAC | 17 | 5143 |
| HSV1-UL30-2111 | − | UCCGGUGGCCGGGCACC | 17 | 5144 |
| HSV1-UL30-2112 | − | CGGUGGCCGGGCACCCG | 17 | 5145 |
| HSV1-UL30-2113 | − | ACCUGUCCACCACCGCC | 17 | 5146 |
| HSV1-UL30-2114 | − | CCUGUCCACCACCGCCC | 17 | 5147 |
| HSV1-UL30-2115 | − | CGGUUCCUGCGACCUCC | 17 | 5148 |
| HSV1-UL30-2116 | − | ACCUCCCCGAAUCCCAC | 17 | 5149 |
| HSV1-UL30-2117 | − | CCCCGAAUCCCACCUGA | 17 | 5150 |
| HSV1-UL30-2118 | − | CUGAACGAGCUGGCGGC | 17 | 5151 |
| HSV1-UL30-2119 | − | UGAACGAGCUGGCGGCC | 17 | 5152 |
| HSV1-UL30-2120 | − | UGCCCACGCCCGUGGUU | 17 | 5153 |
| HSV1-UL30-2121 | − | GCCCACGCCCGUGGUUC | 17 | 5154 |
| HSV1-UL30-2122 | − | GGUUCUGGAAUUCGACA | 17 | 5155 |
| HSV1-UL30-2123 | − | GGAAUUCGACAGCGAAU | 17 | 5156 |
| HSV1-UL30-2124 | − | UGGCCUUCAUGACCCUU | 17 | 5157 |
| HSV1-UL30-2125 | − | UGUGAAACAGUACGGCC | 17 | 5158 |
| HSV1-UL30-2126 | − | CGGCCCCGAGUUCGUGA | 17 | 5159 |
| HSV1-UL30-2127 | − | UCUUGCUGGCCAAGCUG | 17 | 5160 |
| HSV1-UL30-2128 | − | ACAUUUACAAGGUCCCC | 17 | 5161 |
| HSV1-UL30-2129 | − | UUACAAGGUCCCCCUGG | 17 | 5162 |
| HSV1-UL30-2130 | − | UGGACGGGUACGGCCGC | 17 | 5163 |
| HSV1-UL30-2131 | − | UACGGCCGCAUGAACGG | 17 | 5164 |
| HSV1-UL30-2132 | − | ACGGCCGCAUGAACGGC | 17 | 5165 |
| HSV1-UL30-2133 | − | CGGGGCGUGUUUCGCGU | 17 | 5166 |
| HSV1-UL30-2134 | − | GGGGCGUGUUUCGCGUG | 17 | 5167 |
| HSV1-UL30-2135 | − | GCGUGUGGGACAUAGGC | 17 | 5168 |
| HSV1-UL30-2136 | − | UAGGCCAGAGCCACUUC | 17 | 5169 |
| HSV1-UL30-2137 | − | AGCGCAGCAAGAUAAAG | 17 | 5170 |
| HSV1-UL30-2138 | − | UAAAGGUGAACGGCAUG | 17 | 5171 |
| HSV1-UL30-2139 | − | GGUGAACAUCGACAUGU | 17 | 5172 |
| HSV1-UL30-2140 | − | GUGAACAUCGACAUGUA | 17 | 5173 |
| HSV1-UL30-2141 | − | CCGACAAGAUCAAGCUC | 17 | 5174 |
| HSV1-UL30-2142 | − | CAAGCUCAACGCCGUGG | 17 | 5175 |
| HSV1-UL30-2143 | − | CCGUGGCCGAAGCCGUC | 17 | 5176 |
| HSV1-UL30-2144 | − | UGGCCGAAGCCGUCCUG | 17 | 5177 |
| HSV1-UL30-2145 | − | AAGCCGUCCUGAAGGAC | 17 | 5178 |
| HSV1-UL30-2146 | − | CCGUCCUGAAGGACAAG | 17 | 5179 |
| HSV1-UL30-2147 | − | UCCUGAAGGACAAGAAG | 17 | 5180 |
| HSV1-UL30-2148 | − | AGGACAAGAAGAAGGAC | 17 | 5181 |
| HSV1-UL30-2149 | − | CCCCGCCUACUACGCCG | 17 | 5182 |
| HSV1-UL30-2150 | − | CGCCGGGCCCGCGCAAC | 17 | 5183 |
| HSV1-UL30-860 | − | GCCGGGCCCGCGCAACG | 17 | 3798 |
| HSV1-UL30-2152 | − | GCAACGCGGGGUGAUCG | 17 | 5184 |
| HSV1-UL30-2153 | − | UCGGCGAGUACUGCAUA | 17 | 5185 |
| HSV1-UL30-2154 | − | UACAGGAUUCCCUGCUG | 17 | 5186 |
| HSV1-UL30-2155 | − | UUAAGUUUUUGCCCCAU | 17 | 5187 |
| HSV1-UL30-2156 | − | UAAGUUUUUGCCCCAUC | 17 | 5188 |
| HSV1-UL30-2157 | − | CGGCCGUCGCGCGCUUG | 17 | 5189 |
| HSV1-UL30-2158 | − | UGCUGCGCCUGGCCGAC | 17 | 5190 |
| HSV1-UL30-2159 | − | UGCGCCUGGCCGACCAG | 17 | 5191 |
| HSV1-UL30-2160 | − | AGAAGGGCUUUAUUCUG | 17 | 5192 |
| HSV1-UL30-2161 | − | UUAUUCUGCCGGACACC | 17 | 5193 |
| HSV1-UL30-2162 | − | UAUUCUGCCGGACACCC | 17 | 5194 |
| HSV1-UL30-2163 | − | GACACCCAGGGGCGAUU | 17 | 5195 |
| HSV1-UL30-2164 | − | ACACCCAGGGGCGAUUU | 17 | 5196 |
| HSV1-UL30-2165 | − | GGGGCGAUUUAGGGGCG | 17 | 5197 |
| HSV1-UL30-2166 | − | GGGCGAUUUAGGGGCGC | 17 | 5198 |
| HSV1-UL30-2167 | − | GGCGAUUUAGGGGCGCC | 17 | 5199 |
| HSV1-UL30-2168 | − | GCGAUUUAGGGGCGCCG | 17 | 5200 |
| HSV1-UL30-2169 | − | CGAUUUAGGGGCGCCGG | 17 | 5201 |
| HSV1-UL30-2170 | − | GAUUUAGGGGCGCCGGG | 17 | 5202 |
| HSV1-UL30-2171 | − | AUUUAGGGGCGCCGGGG | 17 | 5203 |
| HSV1-UL30-2172 | − | AAGCGUCCGGCCGCAGC | 17 | 5204 |
| HSV1-UL30-2173 | − | AGCGUCCGGCCGCAGCC | 17 | 5205 |
| HSV1-UL30-2174 | − | GCGUCCGGCCGCAGCCC | 17 | 5206 |
| HSV1-UL30-2175 | − | GUCCGGCCGCAGCCCGG | 17 | 5207 |
| HSV1-UL30-2176 | − | GGCCGCAGCCCGGGAGG | 17 | 5208 |
| HSV1-UL30-2177 | − | CCGCAGCCCGGGAGGAC | 17 | 5209 |
| HSV1-UL30-895 | − | CGCAGCCCGGGAGGACG | 17 | 3833 |
| HSV1-UL30-2179 | − | GGAGGACGAGGAGCGGC | 17 | 5210 |
| HSV1-UL30-2180 | − | AGGACGAGGAGCGGCCA | 17 | 5211 |
| HSV1-UL30-897 | − | GGACGAGGAGCGGCCAG | 17 | 3835 |
| HSV1-UL30-2182 | − | ACGAGGAGCGGCCAGAG | 17 | 5212 |
| HSV1-UL30-898 | − | CGAGGAGCGGCCAGAGG | 17 | 3836 |
| HSV1-UL30-2184 | − | AGGAGCGGCCAGAGGAG | 17 | 5213 |
| HSV1-UL30-899 | − | GGAGCGGCCAGAGGAGG | 17 | 3837 |
| HSV1-UL30-900 | − | GAGCGGCCAGAGGAGGA | 17 | 3838 |
| HSV1-UL30-901 | − | AGCGGCCAGAGGAGGAG | 17 | 3839 |
| HSV1-UL30-902 | − | GCGGCCAGAGGAGGAGG | 17 | 3840 |
| HSV1-UL30-2189 | − | GGCCAGAGGAGGAGGGG | 17 | 5214 |
| HSV1-UL30-2190 | − | AGAGGAGGAGGGGGAGG | 17 | 5215 |
| HSV1-UL30-2191 | − | AGGAGGAGGGGGAGGAC | 17 | 5216 |
| HSV1-UL30-2192 | − | GGAGGGGGAGGACGAGG | 17 | 5217 |
| HSV1-UL30-2193 | − | GGAGGACGAGGACGAAC | 17 | 5218 |
| HSV1-UL30-2194 | − | AGGACGAGGACGAACGC | 17 | 5219 |
| HSV1-UL30-905 | − | GGACGAGGACGAACGCG | 17 | 3843 |
| HSV1-UL30-2196 | − | ACGAGGACGAACGCGAG | 17 | 5220 |
| HSV1-UL30-2197 | − | GGACGAACGCGAGGAGG | 17 | 5221 |
| HSV1-UL30-908 | − | GACGAACGCGAGGAGGG | 17 | 3846 |
| HSV1-UL30-909 | − | ACGAACGCGAGGAGGGC | 17 | 3847 |
| HSV1-UL30-2200 | − | ACGCGAGGAGGGCGGGG | 17 | 5222 |
| HSV1-UL30-2201 | − | GAGGAGGGCGGGGGCGA | 17 | 5223 |
| HSV1-UL30-912 | − | AGGAGGGCGGGGGCGAG | 17 | 3850 |
| HSV1-UL30-913 | − | GGAGGGCGGGGGCGAGC | 17 | 3851 |
| HSV1-UL30-2204 | − | GCGGGGGCGAGCGGGAG | 17 | 5224 |
| HSV1-UL30-914 | − | CGGGGGCGAGCGGGAGC | 17 | 3852 |
| HSV1-UL30-2206 | − | GGGGCGAGCGGGAGCCG | 17 | 5225 |
| HSV1-UL30-2207 | − | CGGGAGCCGGAGGGCGC | 17 | 5226 |
| HSV1-UL30-917 | − | GGGAGCCGGAGGGCGCG | 17 | 3855 |
| HSV1-UL30-918 | − | GGAGCCGGAGGGCGCGC | 17 | 3856 |
| HSV1-UL30-2210 | − | AGACCGCCGGCAGGCAC | 17 | 5227 |
| HSV1-UL30-921 | − | GACCGCCGGCAGGCACG | 17 | 3859 |
| HSV1-UL30-2212 | − | GCAGGCACGUGGGGUAC | 17 | 5228 |
| HSV1-UL30-2213 | − | CAGGCACGUGGGGUACC | 17 | 5229 |
| HSV1-UL30-2214 | − | AGGCACGUGGGGUACCA | 17 | 5230 |
| HSV1-UL30-2215 | − | GUGGGGUACCAGGGGGC | 17 | 5231 |
| HSV1-UL30-2216 | − | GGUCCUUGACCCCACUU | 17 | 5232 |
| HSV1-UL30-2217 | − | CCACUUCCGGGUUUCAC | 17 | 5233 |
| HSV1-UL30-2218 | − | GCUUCAGCACGCUCUCC | 17 | 5234 |
| HSV1-UL30-2219 | − | UUCAGCACGCUCUCCCU | 17 | 5235 |
| HSV1-UL30-2220 | − | CCGACGCAGUGGCGCAC | 17 | 5236 |
| HSV1-UL30-2221 | − | CGACGCAGUGGCGCACC | 17 | 5237 |
| HSV1-UL30-2222 | − | CAGUGGCGCACCUGGAG | 17 | 5238 |
| HSV1-UL30-2223 | − | CGCACCUGGAGGCGGGC | 17 | 5239 |
| HSV1-UL30-2224 | − | AGGCGGGCAAGGACUAC | 17 | 5240 |
| HSV1-UL30-2225 | − | GGCGGGCAAGGACUACC | 17 | 5241 |
| HSV1-UL30-2226 | − | CAAGGACUACCUGGAGA | 17 | 5242 |
| HSV1-UL30-2227 | − | ACUACCUGGAGAUCGAG | 17 | 5243 |
| HSV1-UL30-2228 | − | CUACCUGGAGAUCGAGG | 17 | 5244 |
| HSV1-UL30-2229 | − | UACCUGGAGAUCGAGGU | 17 | 5245 |
| HSV1-UL30-2230 | − | ACCUGGAGAUCGAGGUG | 17 | 5246 |
| HSV1-UL30-2231 | − | CCUGGAGAUCGAGGUGG | 17 | 5247 |
| HSV1-UL30-2232 | − | UUCGUCAAGGCUCACGU | 17 | 5248 |
| HSV1-UL30-2233 | − | CGUCAAGGCUCACGUGC | 17 | 5249 |
| HSV1-UL30-2234 | − | UCAAGGCUCACGUGCGA | 17 | 5250 |
| HSV1-UL30-2235 | − | CUCCUCAGCAUCCUCCU | 17 | 5251 |
| HSV1-UL30-2236 | − | UCCUCAGCAUCCUCCUG | 17 | 5252 |
| HSV1-UL30-2237 | − | CGGGACUGGCUCGCCAU | 17 | 5253 |
| HSV1-UL30-2238 | − | CGAAAGCAGAUCCGCUC | 17 | 5254 |
| HSV1-UL30-2239 | − | UCCGCUCGCGGAUUCCC | 17 | 5255 |
| HSV1-UL30-2240 | − | GAUUCCCCAGAGCAGCC | 17 | 5256 |
| HSV1-UL30-2241 | − | UUCCCCAGAGCAGCCCC | 17 | 5257 |
| HSV1-UL30-2242 | − | UCCCCAGAGCAGCCCCG | 17 | 5258 |
| HSV1-UL30-2243 | − | CCGAGGAGGCCGUGCUC | 17 | 5259 |
| HSV1-UL30-2244 | − | CGUGUGUAACUCGGUGU | 17 | 5260 |
| HSV1-UL30-2245 | − | ACUCGGUGUACGGGUUC | 17 | 5261 |
| HSV1-UL30-2246 | − | CUCGGUGUACGGGUUCA | 17 | 5262 |
| HSV1-UL30-2247 | − | UCGGUGUACGGGUUCAC | 17 | 5263 |
| HSV1-UL30-2248 | − | GUUCACGGGAGUGCAGC | 17 | 5264 |
| HSV1-UL30-2249 | − | GGUGACGACCAUCGGCC | 17 | 5265 |
| HSV1-UL30-2250 | − | GAUGCUGCUCGCGACCC | 17 | 5266 |
| HSV1-UL30-2251 | − | GAGUACGUCCACGCGCG | 17 | 5267 |
| HSV1-UL30-2252 | − | CGCGCGCUGGGCGGCCU | 17 | 5268 |
| HSV1-UL30-2253 | − | AGCUCCUGGCCGAUUUC | 17 | 5269 |
| HSV1-UL30-2254 | − | GCUCCUGGCCGAUUUCC | 17 | 5270 |
| HSV1-UL30-2255 | − | GGCCGACAUGCGCGCCC | 17 | 5271 |
| HSV1-UL30-2256 | − | UUCCAUGCGCAUCAUCU | 17 | 5272 |
| HSV1-UL30-2257 | − | UCCAUGCGCAUCAUCUA | 17 | 5273 |
| HSV1-UL30-2258 | − | CCAUGCGCAUCAUCUAC | 17 | 5274 |
| HSV1-UL30-2259 | − | GCAUCAUCUACGGGGAC | 17 | 5275 |
| HSV1-UL30-2260 | − | CCGCGGCCUCACGGCCG | 17 | 5276 |
| HSV1-UL30-2261 | − | CCGCCGGGCUGACGGCC | 17 | 5277 |
| HSV1-UL30-2262 | − | CCGUGGGCGACAAGAUG | 17 | 5278 |
| HSV1-UL30-2263 | − | UCUGCCCCCCAUCAAAC | 17 | 5279 |
| HSV1-UL30-2264 | − | CCCCAUCAAACUCGAGU | 17 | 5280 |
| HSV1-UL30-2265 | − | AGCUGCUGCUGAUCGCC | 17 | 5281 |
| HSV1-UL30-2266 | − | GUACAUCGGCGUCAUCU | 17 | 5282 |
| HSV1-UL30-2267 | − | UACAUCGGCGUCAUCUA | 17 | 5283 |
| HSV1-UL30-2268 | − | ACAUCGGCGUCAUCUAC | 17 | 5284 |
| HSV1-UL30-2269 | − | GGGGUAAGAUGCUCAUC | 17 | 5285 |
| HSV1-UL30-2270 | − | AGAUGCUCAUCAAGGGC | 17 | 5286 |
| HSV1-UL30-2271 | − | UUUAUCAACCGCACCUC | 17 | 5287 |
| HSV1-UL30-2272 | − | UUACGACGAUACCGUCU | 17 | 5288 |
| HSV1-UL30-2273 | − | UACGACGAUACCGUCUC | 17 | 5289 |
| HSV1-UL30-2274 | − | AGCGGCCGCCGCGUUAG | 17 | 5290 |
| HSV1-UL30-2275 | − | CGUUAGCCGAGCGCCCC | 17 | 5291 |
| HSV1-UL30-2276 | − | GUUAGCCGAGCGCCCCG | 17 | 5292 |
| HSV1-UL30-2277 | − | UAGCCGAGCGCCCCGCG | 17 | 5293 |
| HSV1-UL30-1006 | − | AGCCGAGCGCCCCGCGG | 17 | 3944 |
| HSV1-UL30-2279 | − | GCUGGCGCGACCCCUGC | 17 | 5294 |
| HSV1-UL30-2280 | − | UGGCGCGACCCCUGCCC | 17 | 5295 |
| HSV1-UL30-2281 | − | GGCGCGACCCCUGCCCG | 17 | 5296 |
| HSV1-UL30-2282 | − | CGAGGGACUGCAGGCGU | 17 | 5297 |
| HSV1-UL30-2283 | − | GAGGGACUGCAGGCGUU | 17 | 5298 |
| HSV1-UL30-2284 | − | AUCGGCGCAUCACCGAC | 17 | 5299 |
| HSV1-UL30-2285 | − | UCGGCGCAUCACCGACC | 17 | 5300 |
| HSV1-UL30-2286 | − | GGCGCAUCACCGACCCG | 17 | 5301 |
| HSV1-UL30-2287 | − | CGCAUCACCGACCCGGA | 17 | 5302 |
| HSV1-UL30-2288 | − | GCAUCACCGACCCGGAG | 17 | 5303 |
| HSV1-UL30-2289 | − | ACCCGGAGAGGGACAUC | 17 | 5304 |
| HSV1-UL30-2290 | − | GGACUUUGUCCUCACCG | 17 | 5305 |
| HSV1-UL30-2291 | − | UUGUCCUCACCGCCGAA | 17 | 5306 |
| HSV1-UL30-2292 | − | CGCAGGUCCCGUCCAUC | 17 | 5307 |
| HSV1-UL30-2293 | − | GUCCCGUCCAUCAAGGA | 17 | 5308 |
| HSV1-UL30-2294 | − | GAUCGUGGCCCAGACCC | 17 | 5309 |
| HSV1-UL30-2295 | − | GGCCCAGACCCGCGAGG | 17 | 5310 |
| HSV1-UL30-2296 | − | CCCAGACCCGCGAGGUA | 17 | 5311 |
| HSV1-UL30-2297 | − | CCAGACCCGCGAGGUAG | 17 | 5312 |
| HSV1-UL30-2298 | − | GCGGCUGGCCGCCCUCC | 17 | 5313 |
| HSV1-UL30-2299 | − | GCUAGACGCCGCCGCCC | 17 | 5314 |
| HSV1-UL30-2300 | − | CUAGACGCCGCCGCCCC | 17 | 5315 |
| HSV1-UL30-2301 | − | UAGACGCCGCCGCCCCA | 17 | 5316 |
| HSV1-UL30-2302 | − | CGCCGCCGCCCCAGGGG | 17 | 5317 |
| HSV1-UL30-2303 | − | UCCCCGGCCAAGCGCCC | 17 | 5318 |
| HSV1-UL30-1037 | − | CCCCGGCCAAGCGCCCC | 17 | 3975 |
| HSV1-UL30-1038 | − | CCCGGCCAAGCGCCCCC | 17 | 3976 |
| HSV1-UL30-2306 | − | CGUCGCCUGCCGACCCC | 17 | 5319 |
| HSV1-UL30-2307 | − | GUCGCCUGCCGACCCCC | 17 | 5320 |
| HSV1-UL30-2308 | − | UCGCCUGCCGACCCCCC | 17 | 5321 |
| HSV1-UL30-2309 | − | CCGCAAGCUGCUGGUGU | 17 | 5322 |
| HSV1-UL30-2310 | − | GCUGGUGUCCGAGCUGG | 17 | 5323 |
| HSV1-UL30-2311 | − | UGGUGUCCGAGCUGGCC | 17 | 5324 |
| HSV1-UL30-2312 | − | UUGCCCACGGCGUCGCC | 17 | 5325 |
| HSV1-UL30-2313 | − | ACGGCGUCGCCCUGAAC | 17 | 5326 |
| HSV1-UL30-2314 | − | AUUACUUCUCCCACCUG | 17 | 5327 |
| HSV1-UL30-2315 | − | UUACUUCUCCCACCUGU | 17 | 5328 |
| HSV1-UL30-2316 | − | UACUUCUCCCACCUGUU | 17 | 5329 |
| HSV1-UL30-2317 | − | GACAUUCAAGGCCCUGU | 17 | 5330 |
| HSV1-UL30-2318 | − | ACAUUCAAGGCCCUGUU | 17 | 5331 |
| HSV1-UL30-2319 | − | CAUUCAAGGCCCUGUUU | 17 | 5332 |
| HSV1-UL30-2320 | − | GAAUAACGCCAAGAUCA | 17 | 5333 |
| HSV1-UL30-2321 | − | AUAACGCCAAGAUCACC | 17 | 5334 |
| HSV1-UL30-2322 | − | GUUAAAAAGGUUUAUUC | 17 | 5335 |
| HSV1-UL30-2323 | − | CCGAAGUGUGGCACCCC | 17 | 5336 |
| HSV1-UL30-2324 | − | GACGUGGCCGCGCGGCU | 17 | 5337 |
| HSV1-UL30-2325 | − | CGCGCGGCUCCGGACCG | 17 | 5338 |
| HSV1-UL30-2326 | − | GCUCCGGACCGCAGGGU | 17 | 5339 |
| HSV1-UL30-2327 | − | CUCCGGACCGCAGGGUU | 17 | 5340 |
| HSV1-UL30-2328 | − | CCGCAGGGUUCGGGGCG | 17 | 5341 |
| HSV1-UL30-2329 | − | UGGGUGCCGGCGCUACG | 17 | 5342 |
| HSV1-UL30-2330 | − | GGGUGCCGGCGCUACGG | 17 | 5343 |
| HSV1-UL30-2331 | − | GUGCCGGCGCUACGGCG | 17 | 5344 |
| HSV1-UL30-2332 | − | UGCCGGCGCUACGGCGG | 17 | 5345 |
| HSV1-UL30-2333 | − | ACGGCGGAGGAAACUCG | 17 | 5346 |
| HSV1-UL30-2334 | − | ACUCGUCGAAUGUUGCA | 17 | 5347 |
| HSV1-UL30-2335 | + | AAGGCUCUAUGCAACAUUCG | 20 | 5348 |
| HSV1-UL30-2336 | + | UAGCGCCGGCACCCACCGCC | 20 | 5349 |
| HSV1-UL30-2337 | + | ACCGCCCCGAACCCUGCGGU | 20 | 5350 |
| HSV1-UL30-2338 | + | CCGCCCCGAACCCUGCGGUC | 20 | 5351 |
| HSV1-UL30-2339 | + | GAGCCGCGCGGCCACGUCGU | 20 | 5352 |
| HSV1-UL30-2340 | + | AGCCGCGCGGCCACGUCGUC | 20 | 5353 |
| HSV1-UL30-2341 | + | GCCGCGCGGCCACGUCGUCC | 20 | 5354 |
| HSV1-UL30-2342 | + | CCGCGCGGCCACGUCGUCCG | 20 | 5355 |
| HSV1-UL30-2343 | + | CGUCCGGGGGGUGCCACACU | 20 | 5356 |
| HSV1-UL30-2344 | + | GUCCGGGGGGUGCCACACUU | 20 | 5357 |
| HSV1-UL30-2345 | + | UCCGGGGGGUGCCACACUUC | 20 | 5358 |
| HSV1-UL30-2346 | + | AUCUUGGCGUUAUUCCCAAA | 20 | 5359 |
| HSV1-UL30-2347 | + | CGUUAUUCCCAAACAGGGCC | 20 | 5360 |
| HSV1-UL30-2348 | + | ACGCACGCCGCCCCCAACAG | 20 | 5361 |
| HSV1-UL30-1089 | + | CGCACGCCGCCCCCAACAGG | 20 | 4027 |
| HSV1-UL30-2350 | + | GCACGCCGCCCCCAACAGGU | 20 | 5362 |
| HSV1-UL30-2351 | + | ACGCCGCCCCCAACAGGUGG | 20 | 5363 |
| HSV1-UL30-2352 | + | GAGAAGUAAUAGUCCGUGUU | 20 | 5364 |
| HSV1-UL30-2353 | + | UCCGUGUUCAGGGCGACGCC | 20 | 5365 |
| HSV1-UL30-2354 | + | CGCCGUGGGCAAUGGCGUAU | 20 | 5366 |
| HSV1-UL30-2355 | + | GCCGUGGGCAAUGGCGUAUG | 20 | 5367 |
| HSV1-UL30-2356 | + | AUGCGGGAUCCUCGGCCAGC | 20 | 5368 |
| HSV1-UL30-2357 | + | AGCUCGGACACCAGCAGCUU | 20 | 5369 |
| HSV1-UL30-2358 | + | GCUCGGACACCAGCAGCUUG | 20 | 5370 |
| HSV1-UL30-2359 | + | ACACCAGCAGCUUGCGGGGC | 20 | 5371 |
| HSV1-UL30-2360 | + | GCGGGGCUUGGACGCGCCUC | 20 | 5372 |
| HSV1-UL30-2361 | + | CGGGGCUUGGACGCGCCUCC | 20 | 5373 |
| HSV1-UL30-2362 | + | GGGGCUUGGACGCGCCUCCC | 20 | 5374 |
| HSV1-UL30-2363 | + | GGGCUUGGACGCGCCUCCCG | 20 | 5375 |
| HSV1-UL30-2364 | + | UCGGCAGGCGACGGCGUCUC | 20 | 5376 |
| HSV1-UL30-2365 | + | CGGCAGGCGACGGCGUCUCC | 20 | 5377 |
| HSV1-UL30-2366 | + | GGCAGGCGACGGCGUCUCCC | 20 | 5378 |
| HSV1-UL30-2367 | + | CGUCUCCCGGGGGCGCUUGG | 20 | 5379 |
| HSV1-UL30-2368 | + | GUCUCCCGGGGGCGCUUGGC | 20 | 5380 |
| HSV1-UL30-2369 | + | UCUCCCGGGGGCGCUUGGCC | 20 | 5381 |
| HSV1-UL30-2370 | + | CUCCCGGGGGCGCUUGGCCG | 20 | 5382 |
| HSV1-UL30-2371 | + | CCCGGGGGCGCUUGGCCGGG | 20 | 5383 |
| HSV1-UL30-2372 | + | GGGCGCUUGGCCGGGGAGGG | 20 | 5384 |
| HSV1-UL30-2373 | + | UGGCCGGGGAGGGCAGGGCC | 20 | 5385 |
| HSV1-UL30-1124 | + | GGCCGGGGAGGGCAGGGCCG | 20 | 4062 |
| HSV1-UL30-1125 | + | GCCGGGGAGGGCAGGGCCGC | 20 | 4063 |
| HSV1-UL30-1126 | + | CCGGGGAGGGCAGGGCCGCG | 20 | 4064 |
| HSV1-UL30-1127 | + | CGGGGAGGGCAGGGCCGCGG | 20 | 4065 |
| HSV1-UL30-1128 | + | GGGGAGGGCAGGGCCGCGGG | 20 | 4066 |
| HSV1-UL30-2379 | + | AGGGCAGGGCCGCGGGGGGG | 20 | 5386 |
| HSV1-UL30-2380 | + | GGGGGGGGCGGGCUCGUCCC | 20 | 5387 |
| HSV1-UL30-2381 | + | GGGGGGGCGGGCUCGUCCCC | 20 | 5388 |
| HSV1-UL30-2382 | + | GGGGCGGCGGCGUCUAGCUC | 20 | 5389 |
| HSV1-UL30-2383 | + | GGGCGGCGGCGUCUAGCUCG | 20 | 5390 |
| HSV1-UL30-2384 | + | GCGGCGGCGUCUAGCUCGCG | 20 | 5391 |
| HSV1-UL30-2385 | + | GCGACCGUCUCCUCUACCUC | 20 | 5392 |
| HSV1-UL30-2386 | + | GUCUCCUCUACCUCGCGGGU | 20 | 5393 |
| HSV1-UL30-2387 | + | GGUCUGGGCCACGAUCACGU | 20 | 5394 |
| HSV1-UL30-2388 | + | GUCUGGGCCACGAUCACGUA | 20 | 5395 |
| HSV1-UL30-2389 | + | CGUACGGGAUCCGGUCCUUG | 20 | 5396 |
| HSV1-UL30-2390 | + | CGGGAUCCGGUCCUUGAUGG | 20 | 5397 |
| HSV1-UL30-2391 | + | GGGAUCCGGUCCUUGAUGGA | 20 | 5398 |
| HSV1-UL30-2392 | + | AUGGACGGGACCUGCGCGCG | 20 | 5399 |
| HSV1-UL30-2393 | + | GGACCUGCGCGCGGCGGGCC | 20 | 5400 |
| HSV1-UL30-2394 | + | AGCUUGUAAUACACCGUCAG | 20 | 5401 |
| HSV1-UL30-2395 | + | GCGCUUGUUGGUGUACGCGC | 20 | 5402 |
| HSV1-UL30-2396 | + | GGUGUCUGCUCAGUUCGGCG | 20 | 5403 |
| HSV1-UL30-2397 | + | UGUCUGCUCAGUUCGGCGGU | 20 | 5404 |
| HSV1-UL30-2398 | + | UCGGCGGUGAGGACAAAGUC | 20 | 5405 |
| HSV1-UL30-2399 | + | AAAGUCCUGGAUGUCCCUCU | 20 | 5406 |
| HSV1-UL30-2400 | + | UCCGGGUCGGUGAUGCGCCG | 20 | 5407 |
| HSV1-UL30-2401 | + | UGAUGCGCCGAUGGGCGUCU | 20 | 5408 |
| HSV1-UL30-2402 | + | AUGCGCCGAUGGGCGUCUAC | 20 | 5409 |
| HSV1-UL30-2403 | + | GGGCGUCUACGAGGACGGCC | 20 | 5410 |
| HSV1-UL30-2404 | + | CCCCGAACGCCUGCAGUCCC | 20 | 5411 |
| HSV1-UL30-2405 | + | AACGCCUGCAGUCCCUCGGG | 20 | 5412 |
| HSV1-UL30-2406 | + | ACGCCUGCAGUCCCUCGGGC | 20 | 5413 |
| HSV1-UL30-2407 | + | GUCGCGCCAGCCACUCCUCC | 20 | 5414 |
| HSV1-UL30-2408 | + | UCGCGCCAGCCACUCCUCCG | 20 | 5415 |
| HSV1-UL30-2409 | + | CGGCUAACGCGGCGGCCGCU | 20 | 5416 |
| HSV1-UL30-2410 | + | GGCUAACGCGGCGGCCGCUC | 20 | 5417 |
| HSV1-UL30-2411 | + | UCGUAAAACAGCAGGUCGAC | 20 | 5418 |
| HSV1-UL30-2412 | + | ACAGCAGGUCGACCAGGGCC | 20 | 5419 |
| HSV1-UL30-2413 | + | CAGCAGGUCGACCAGGGCCC | 20 | 5420 |
| HSV1-UL30-2414 | + | GCACCAGAUCCACGCCCUUG | 20 | 5421 |
| HSV1-UL30-2415 | + | UGGCGAUCAGCAGCAGCUUG | 20 | 5422 |
| HSV1-UL30-2416 | + | UGGUGAACGUCUUUUCGCAC | 20 | 5423 |
| HSV1-UL30-2417 | + | UCUUUUCGCACUCGAGUUUG | 20 | 5424 |
| HSV1-UL30-2418 | + | CUUUUCGCACUCGAGUUUGA | 20 | 5425 |
| HSV1-UL30-2419 | + | UUUUCGCACUCGAGUUUGAU | 20 | 5426 |
| HSV1-UL30-2420 | + | UUUCGCACUCGAGUUUGAUG | 20 | 5427 |
| HSV1-UL30-2421 | + | CACUCGAGUUUGAUGGGGGG | 20 | 5428 |
| HSV1-UL30-2422 | + | GGGGGGCAGAAACAGCGCGC | 20 | 5429 |
| HSV1-UL30-2423 | + | CGGCCGUCAGCCCGGCGGCC | 20 | 5430 |
| HSV1-UL30-2424 | + | GGCCGCGGCACAGCACAAAG | 20 | 5431 |
| HSV1-UL30-1205 | + | GCCGCGGCACAGCACAAAGA | 20 | 4143 |
| HSV1-UL30-2426 | + | UGUCCCCGUAGAUGAUGCGC | 20 | 5432 |
| HSV1-UL30-2427 | + | GUCCCCGUAGAUGAUGCGCA | 20 | 5433 |
| HSV1-UL30-2428 | + | CGUAGAUGAUGCGCAUGGAA | 20 | 5434 |
| HSV1-UL30-2429 | + | UGAUGCGCAUGGAAUAGGGC | 20 | 5435 |
| HSV1-UL30-2430 | + | GAUGCGCAUGGAAUAGGGCC | 20 | 5436 |
| HSV1-UL30-2431 | + | AUGCGCAUGGAAUAGGGCCC | 20 | 5437 |
| HSV1-UL30-2432 | + | GGCGCGCAUGUCGGCCGCCU | 20 | 5438 |
| HSV1-UL30-2433 | + | GCGCGCAUGUCGGCCGCCUC | 20 | 5439 |
| HSV1-UL30-2434 | + | CGCGCAUGUCGGCCGCCUCC | 20 | 5440 |
| HSV1-UL30-2435 | + | GCCGCCUCCGGGAAAUCGGC | 20 | 5441 |
| HSV1-UL30-2436 | + | CCGCCUCCGGGAAAUCGGCC | 20 | 5442 |
| HSV1-UL30-2437 | + | GGAAAUCGGCCAGGAGCUGU | 20 | 5443 |
| HSV1-UL30-2438 | + | UCGAAGGCCGCCCAGCGCGC | 20 | 5444 |
| HSV1-UL30-2439 | + | CAGCGCGCGUGGACGUACUC | 20 | 5445 |
| HSV1-UL30-2440 | + | CGUGGACGUACUCGCGGGUC | 20 | 5446 |
| HSV1-UL30-2441 | + | GCGGCAACGUGCAGGCACGG | 20 | 5447 |
| HSV1-UL30-2442 | + | CGGCAACGUGCAGGCACGGC | 20 | 5448 |
| HSV1-UL30-2443 | + | GGAGUCCGUGCUGCACUCCC | 20 | 5449 |
| HSV1-UL30-2444 | + | CACUCCCGUGAACCCGUACA | 20 | 5450 |
| HSV1-UL30-2445 | + | AUGGCGGCCUGCUGCUUGUC | 20 | 5451 |
| HSV1-UL30-2446 | + | UGGCGGCCUGCUGCUUGUCC | 20 | 5452 |
| HSV1-UL30-2447 | + | UGUCCAGGAGCACGGCCUCC | 20 | 5453 |
| HSV1-UL30-2448 | + | GUCCAGGAGCACGGCCUCCU | 20 | 5454 |
| HSV1-UL30-2449 | + | ACGGCCUCCUCGGGGCUGCU | 20 | 5455 |
| HSV1-UL30-2450 | + | CGGCCUCCUCGGGGCUGCUC | 20 | 5456 |
| HSV1-UL30-2451 | + | GGCCUCCUCGGGGCUGCUCU | 20 | 5457 |
| HSV1-UL30-2452 | + | GCCUCCUCGGGGCUGCUCUG | 20 | 5458 |
| HSV1-UL30-2453 | + | GGGGCUGCUCUGGGGAAUCC | 20 | 5459 |
| HSV1-UL30-2454 | + | CUGCUCUGGGGAAUCCGCGA | 20 | 5460 |
| HSV1-UL30-2455 | + | AGCGGAUCUGCUUUCGCAUG | 20 | 5461 |
| HSV1-UL30-2456 | + | CGCAUGGCGAGCCAGUCCCG | 20 | 5462 |
| HSV1-UL30-2457 | + | GCAUGGCGAGCCAGUCCCGC | 20 | 5463 |
| HSV1-UL30-2458 | + | AUGGCGAGCCAGUCCCGCAG | 20 | 5464 |
| HSV1-UL30-2459 | + | GCCAGUCCCGCAGGAGGAUG | 20 | 5465 |
| HSV1-UL30-2460 | + | CAGUCCCGCAGGAGGAUGCU | 20 | 5466 |
| HSV1-UL30-2461 | + | AGUCCCGCAGGAGGAUGCUG | 20 | 5467 |
| HSV1-UL30-2462 | + | CUGAGGAGGCUCUCUCGCAC | 20 | 5468 |
| HSV1-UL30-2463 | + | UCUCUCGCACGUGAGCCUUG | 20 | 5469 |
| HSV1-UL30-2464 | + | CUCGCACGUGAGCCUUGACG | 20 | 5470 |
| HSV1-UL30-2465 | + | UGCGCCACUGCGUCGGCCCU | 20 | 5471 |
| HSV1-UL30-2466 | + | GCGCCACUGCGUCGGCCCUC | 20 | 5472 |
| HSV1-UL30-2467 | + | CGCCACUGCGUCGGCCCUCA | 20 | 5473 |
| HSV1-UL30-2468 | + | CCACUGCGUCGGCCCUCAGG | 20 | 5474 |
| HSV1-UL30-2469 | + | CGGCCCUCAGGGAGAGCGUG | 20 | 5475 |
| HSV1-UL30-2470 | + | AGCGUGCUGAAGCACAGGUU | 20 | 5476 |
| HSV1-UL30-2471 | + | CUGAAGCACAGGUUGUGGGC | 20 | 5477 |
| HSV1-UL30-2472 | + | GGUUGUGGGCCUGGAUGAUG | 20 | 5478 |
| HSV1-UL30-2473 | + | GUUGUGGGCCUGGAUGAUGC | 20 | 5479 |
| HSV1-UL30-2474 | + | UGGGGUACAGGCUGGCAAAG | 20 | 5480 |
| HSV1-UL30-2475 | + | UGGCAAAGUCGAACACCACC | 20 | 5481 |
| HSV1-UL30-2476 | + | GGCAAAGUCGAACACCACCA | 20 | 5482 |
| HSV1-UL30-2477 | + | AACACCACCACGGGGUUCAC | 20 | 5483 |
| HSV1-UL30-2478 | + | CCACGGGGUUCACGUGAAAC | 20 | 5484 |
| HSV1-UL30-2479 | + | CACGGGGUUCACGUGAAACC | 20 | 5485 |
| HSV1-UL30-2480 | + | GGUUCACGUGAAACCCGGAA | 20 | 5486 |
| HSV1-UL30-2481 | + | GUUCACGUGAAACCCGGAAG | 20 | 5487 |
| HSV1-UL30-2482 | + | UGAAACCCGGAAGUGGGGUC | 20 | 5488 |
| HSV1-UL30-2483 | + | UCUGGCCGCUCCUCGUCCUC | 20 | 5489 |
| HSV1-UL30-2484 | + | CUCGUCCUCCCGGGCUGCGG | 20 | 5490 |
| HSV1-UL30-2485 | + | CCCGGGCUGCGGCCGGACGC | 20 | 5491 |
| HSV1-UL30-2486 | + | CCGGCGCCCCUAAAUCGCCC | 20 | 5492 |
| HSV1-UL30-2487 | + | AAUCGCCCCUGGGUGUCCGG | 20 | 5493 |
| HSV1-UL30-2488 | + | CGCAGCAGGCACGUAAAGAC | 20 | 5494 |
| HSV1-UL30-2489 | + | UGCUGGCCGUCGUAGAUGGU | 20 | 5495 |
| HSV1-UL30-2490 | + | ACCCGCCAAGCGCGCGACGG | 20 | 5496 |
| HSV1-UL30-2491 | + | CCGCCAAGCGCGCGACGGCC | 20 | 5497 |
| HSV1-UL30-2492 | + | GCGACGGCCGAGAGCUCCAG | 20 | 5498 |
| HSV1-UL30-2493 | + | CGACGGCCGAGAGCUCCAGA | 20 | 5499 |
| HSV1-UL30-2494 | + | AAAAACAGCUGGCCCACCAG | 20 | 5500 |
| HSV1-UL30-2495 | + | AAAACAGCUGGCCCACCAGC | 20 | 5501 |
| HSV1-UL30-2496 | + | AAACAGCUGGCCCACCAGCA | 20 | 5502 |
| HSV1-UL30-2497 | + | CGCCGAUCACCCCGCGUUGC | 20 | 5503 |
| HSV1-UL30-2498 | + | CGGGCCCGGCGGCGUAGUAG | 20 | 5504 |
| HSV1-UL30-2499 | + | GGGCCCGGCGGCGUAGUAGG | 20 | 5505 |
| HSV1-UL30-2500 | + | GGCCCGGCGGCGUAGUAGGC | 20 | 5506 |
| HSV1-UL30-2501 | + | AGGUCCUUCUUCUUGUCCUU | 20 | 5507 |
| HSV1-UL30-2502 | + | GGACGGCUUCGGCCACGGCG | 20 | 5508 |
| HSV1-UL30-2503 | + | CACGGCGUUGAGCUUGUAGC | 20 | 5509 |
| HSV1-UL30-2504 | + | CGGCGUUGAGCUUGUAGCUC | 20 | 5510 |
| HSV1-UL30-2505 | + | ACCUUUAUCUUGCUGCGCUU | 20 | 5511 |
| HSV1-UL30-2506 | + | CCUUUAUCUUGCUGCGCUUC | 20 | 5512 |
| HSV1-UL30-2507 | + | CUCUGGCCUAUGUCCCACAC | 20 | 5513 |
| HSV1-UL30-2508 | + | UUCAUGCGGCCGUACCCGUC | 20 | 5514 |
| HSV1-UL30-2509 | + | UCAUGCGGCCGUACCCGUCC | 20 | 5515 |
| HSV1-UL30-2510 | + | CAUGCGGCCGUACCCGUCCA | 20 | 5516 |
| HSV1-UL30-2511 | + | AUGCGGCCGUACCCGUCCAG | 20 | 5517 |
| HSV1-UL30-2512 | + | UGUCCGUCAGCUUGGCCAGC | 20 | 5518 |
| HSV1-UL30-2513 | + | CCGUCAGCUUGGCCAGCAAG | 20 | 5519 |
| HSV1-UL30-2514 | + | UGGCCAGCAAGAAGGGCCAG | 20 | 5520 |
| HSV1-UL30-2515 | + | UGAUGAUGUUGUACCCGGUC | 20 | 5521 |
| HSV1-UL30-2516 | + | UGUUGUACCCGGUCACGAAC | 20 | 5522 |
| HSV1-UL30-2517 | + | GUUGUACCCGGUCACGAACU | 20 | 5523 |
| HSV1-UL30-2518 | + | UCGGGGCCGUACUGUUUCAC | 20 | 5524 |
| HSV1-UL30-2519 | + | CGUACUGUUUCACAAGGGUC | 20 | 5525 |
| HSV1-UL30-2520 | + | UCAUGAAGGCCAACAGCAUC | 20 | 5526 |
| HSV1-UL30-2521 | + | ACAGCAUCUCGAAUUCGCUG | 20 | 5527 |
| HSV1-UL30-2522 | + | UCGAAUUCGCUGUCGAAUUC | 20 | 5528 |
| HSV1-UL30-2523 | + | CGCUGUCGAAUUCCAGAACC | 20 | 5529 |
| HSV1-UL30-2524 | + | CGAAUUCCAGAACCACGGGC | 20 | 5530 |
| HSV1-UL30-2525 | + | CUGGCCGCCAGCUCGUUCAG | 20 | 5531 |
| HSV1-UL30-2526 | + | UGGCCGCCAGCUCGUUCAGG | 20 | 5532 |
| HSV1-UL30-2527 | + | CCAGCUCGUUCAGGUGGGAU | 20 | 5533 |
| HSV1-UL30-2528 | + | CAGCUCGUUCAGGUGGGAUU | 20 | 5534 |
| HSV1-UL30-2529 | + | AGCUCGUUCAGGUGGGAUUC | 20 | 5535 |
| HSV1-UL30-2530 | + | GCUCGUUCAGGUGGGAUUCG | 20 | 5536 |
| HSV1-UL30-2531 | + | GGUGGGAUUCGGGGAGGUCG | 20 | 5537 |
| HSV1-UL30-2532 | + | GUGGGAUUCGGGGAGGUCGC | 20 | 5538 |
| HSV1-UL30-2533 | + | AUUCGGGGAGGUCGCAGGAA | 20 | 5539 |
| HSV1-UL30-2534 | + | GGGGAGGUCGCAGGAACCGA | 20 | 5540 |
| HSV1-UL30-2535 | + | UCGCAGGAACCGAGCGAAAA | 20 | 5541 |
| HSV1-UL30-1345 | + | CGCAGGAACCGAGCGAAAAC | 20 | 4283 |
| HSV1-UL30-2537 | + | CAGGAACCGAGCGAAAACAG | 20 | 5542 |
| HSV1-UL30-2538 | + | GAAAACAGGAGGACGUGCUC | 20 | 5543 |
| HSV1-UL30-2539 | + | GGACGUGCUCCAGGGCGGUG | 20 | 5544 |
| HSV1-UL30-2540 | + | GGGCGGUGGUGGACAGGUCG | 20 | 5545 |
| HSV1-UL30-2541 | + | UGGACAGGUCGUAGAGCAGA | 20 | 5546 |
| HSV1-UL30-2542 | + | UCGUAGAGCAGACAGGAUAU | 20 | 5547 |
| HSV1-UL30-2543 | + | UAUCUGGAUGACCAGGUCCU | 20 | 5548 |
| HSV1-UL30-2544 | + | GUCCUCCGGGUGCCCGGCCA | 20 | 5549 |
| HSV1-UL30-2545 | + | UCCUCCGGGUGCCCGGCCAC | 20 | 5550 |
| HSV1-UL30-2546 | + | CCCCCGCCUUGCAUUCGAUA | 20 | 5551 |
| HSV1-UL30-2547 | + | UGCAUUCGAUAUCGAAGCAC | 20 | 5552 |
| HSV1-UL30-2548 | + | UACAGUUAAACUCGACGUCG | 20 | 5553 |
| HSV1-UL30-2549 | + | ACUCGACGUCGCUGGAUGUC | 20 | 5554 |
| HSV1-UL30-2550 | + | GCUGGAUGUCCCGAAGGCCA | 20 | 5555 |
| HSV1-UL30-2551 | + | CUGGAUGUCCCGAAGGCCAU | 20 | 5556 |
| HSV1-UL30-2552 | + | AAGGCCAUCGGGGCCGCCGG | 20 | 5557 |
| HSV1-UL30-2553 | + | GCGUGUUGUUCCGGCCCGGU | 20 | 5558 |
| HSV1-UL30-2554 | + | CCGGUUUGAGACGGUACCAG | 20 | 5559 |
| HSV1-UL30-2555 | + | GACGGUACCAGCCGAAGGUG | 20 | 5560 |
| HSV1-UL30-2556 | + | ACCAGCCGAAGGUGACGAAC | 20 | 5561 |
| HSV1-UL30-2557 | + | CCAGCCGAAGGUGACGAACC | 20 | 5562 |
| HSV1-UL30-2558 | + | GUGACGAACCCGGGGUUGUC | 20 | 5563 |
| HSV1-UL30-2559 | + | CGAACCCGGGGUUGUCCAGG | 20 | 5564 |
| HSV1-UL30-2560 | + | CCGGGGUUGUCCAGGAUGAA | 20 | 5565 |
| HSV1-UL30-2561 | + | ACCCUCGUACUUCUUGAUGG | 20 | 5566 |
| HSV1-UL30-2562 | + | CGUACUUCUUGAUGGCCGGG | 20 | 5567 |
| HSV1-UL30-2563 | + | UACGACAGCACACGCCCGCU | 20 | 5568 |
| HSV1-UL30-2564 | + | CGGACGUAGACGCGGUAAAA | 20 | 5569 |
| HSV1-UL30-2565 | + | CGUAGACGCGGUAAAACAGA | 20 | 5570 |
| HSV1-UL30-2566 | + | GUAGACGCGGUAAAACAGAG | 20 | 5571 |
| HSV1-UL30-2567 | + | UGCGCUCCACCACCUCCGCC | 20 | 5572 |
| HSV1-UL30-2568 | + | CCUCCGCCUCGAAGUGGUCC | 20 | 5573 |
| HSV1-UL30-2569 | + | CUCCGCCUCGAAGUGGUCCG | 20 | 5574 |
| HSV1-UL30-2570 | + | AAGUGGUCCGCGGAGAUGCC | 20 | 5575 |
| HSV1-UL30-2571 | + | AGUGGUCCGCGGAGAUGCCG | 20 | 5576 |
| HSV1-UL30-2572 | + | GAUGCCGCGGAACGACGCGC | 20 | 5577 |
| HSV1-UL30-2573 | + | AUGCCGCGGAACGACGCGCC | 20 | 5578 |
| HSV1-UL30-2574 | + | UGCCGCGGAACGACGCGCCC | 20 | 5579 |
| HSV1-UL30-2575 | + | GACGCGCCCGGGGACUCGCG | 20 | 5580 |
| HSV1-UL30-2576 | + | GGGCCGCGGCCAUGCGCUCG | 20 | 5581 |
| HSV1-UL30-2577 | + | CAUGCGCUCGCAGAGAUCUC | 20 | 5582 |
| HSV1-UL30-2578 | + | AUGCGCUCGCAGAGAUCUCG | 20 | 5583 |
| HSV1-UL30-2579 | + | UACUGCCGCGUGCCGUAAAC | 20 | 5584 |
| HSV1-UL30-2580 | + | AACGGCCACCCGGUGGCCUU | 20 | 5585 |
| HSV1-UL30-2581 | + | ACGGCCACCCGGUGGCCUUC | 20 | 5586 |
| HSV1-UL30-2582 | + | UGGCCUUCCGGAGUCAGGCC | 20 | 5587 |
| HSV1-UL30-2583 | + | GGCCUUCCGGAGUCAGGCCC | 20 | 5588 |
| HSV1-UL30-2584 | + | GCUCUAUGCAACAUUCG | 17 | 5589 |
| HSV1-UL30-2585 | + | CGCCGGCACCCACCGCC | 17 | 5590 |
| HSV1-UL30-2586 | + | GCCCCGAACCCUGCGGU | 17 | 5591 |
| HSV1-UL30-2587 | + | CCCCGAACCCUGCGGUC | 17 | 5592 |
| HSV1-UL30-2588 | + | CCGCGCGGCCACGUCGU | 17 | 5593 |
| HSV1-UL30-2589 | + | CGCGCGGCCACGUCGUC | 17 | 5594 |
| HSV1-UL30-2590 | + | GCGCGGCCACGUCGUCC | 17 | 5595 |
| HSV1-UL30-2591 | + | CGCGGCCACGUCGUCCG | 17 | 5596 |
| HSV1-UL30-2592 | + | CCGGGGGGUGCCACACU | 17 | 5597 |
| HSV1-UL30-2593 | + | CGGGGGGUGCCACACUU | 17 | 5598 |
| HSV1-UL30-2594 | + | GGGGGGUGCCACACUUC | 17 | 5599 |
| HSV1-UL30-2595 | + | UUGGCGUUAUUCCCAAA | 17 | 5600 |
| HSV1-UL30-2596 | + | UAUUCCCAAACAGGGCC | 17 | 5601 |
| HSV1-UL30-2597 | + | CACGCCGCCCCCAACAG | 17 | 5602 |
| HSV1-UL30-1438 | + | ACGCCGCCCCCAACAGG | 17 | 4376 |
| HSV1-UL30-2599 | + | CGCCGCCCCCAACAGGU | 17 | 5603 |
| HSV1-UL30-2600 | + | CCGCCCCCAACAGGUGG | 17 | 5604 |
| HSV1-UL30-2601 | + | AAGUAAUAGUCCGUGUU | 17 | 5605 |
| HSV1-UL30-2602 | + | GUGUUCAGGGCGACGCC | 17 | 5606 |
| HSV1-UL30-2603 | + | CGUGGGCAAUGGCGUAU | 17 | 5607 |
| HSV1-UL30-2604 | + | GUGGGCAAUGGCGUAUG | 17 | 5608 |
| HSV1-UL30-2605 | + | CGGGAUCCUCGGCCAGC | 17 | 5609 |
| HSV1-UL30-2606 | + | UCGGACACCAGCAGCUU | 17 | 5610 |
| HSV1-UL30-2607 | + | CGGACACCAGCAGCUUG | 17 | 5611 |
| HSV1-UL30-2608 | + | CCAGCAGCUUGCGGGGC | 17 | 5612 |
| HSV1-UL30-2609 | + | GGGCUUGGACGCGCCUC | 17 | 5613 |
| HSV1-UL30-2610 | + | GGCUUGGACGCGCCUCC | 17 | 5614 |
| HSV1-UL30-2611 | + | GCUUGGACGCGCCUCCC | 17 | 5615 |
| HSV1-UL30-2612 | + | CUUGGACGCGCCUCCCG | 17 | 5616 |
| HSV1-UL30-2613 | + | GCAGGCGACGGCGUCUC | 17 | 5617 |
| HSV1-UL30-2614 | + | CAGGCGACGGCGUCUCC | 17 | 5618 |
| HSV1-UL30-2615 | + | AGGCGACGGCGUCUCCC | 17 | 5619 |
| HSV1-UL30-2616 | + | CUCCCGGGGGCGCUUGG | 17 | 5620 |
| HSV1-UL30-2617 | + | UCCCGGGGGCGCUUGGC | 17 | 5621 |
| HSV1-UL30-2618 | + | CCCGGGGGCGCUUGGCC | 17 | 5622 |
| HSV1-UL30-2619 | + | CCGGGGGCGCUUGGCCG | 17 | 5623 |
| HSV1-UL30-2620 | + | GGGGGCGCUUGGCCGGG | 17 | 5624 |
| HSV1-UL30-2621 | + | CGCUUGGCCGGGGAGGG | 17 | 5625 |
| HSV1-UL30-2622 | + | CCGGGGAGGGCAGGGCC | 17 | 5626 |
| HSV1-UL30-1473 | + | CGGGGAGGGCAGGGCCG | 17 | 4411 |
| HSV1-UL30-1474 | + | GGGGAGGGCAGGGCCGC | 17 | 4412 |
| HSV1-UL30-1475 | + | GGGAGGGCAGGGCCGCG | 17 | 4413 |
| HSV1-UL30-1476 | + | GGAGGGCAGGGCCGCGG | 17 | 4414 |
| HSV1-UL30-1477 | + | GAGGGCAGGGCCGCGGG | 17 | 4415 |
| HSV1-UL30-2628 | + | GCAGGGCCGCGGGGGGG | 17 | 5627 |
| HSV1-UL30-2629 | + | GGGGGCGGGCUCGUCCC | 17 | 5628 |
| HSV1-UL30-2630 | + | GGGGCGGGCUCGUCCCC | 17 | 5629 |
| HSV1-UL30-2631 | + | GCGGCGGCGUCUAGCUC | 17 | 5630 |
| HSV1-UL30-2632 | + | CGGCGGCGUCUAGCUCG | 17 | 5631 |
| HSV1-UL30-2633 | + | GCGGCGUCUAGCUCGCG | 17 | 5632 |
| HSV1-UL30-2634 | + | ACCGUCUCCUCUACCUC | 17 | 5633 |
| HSV1-UL30-2635 | + | UCCUCUACCUCGCGGGU | 17 | 5634 |
| HSV1-UL30-2636 | + | CUGGGCCACGAUCACGU | 17 | 5635 |
| HSV1-UL30-2637 | + | UGGGCCACGAUCACGUA | 17 | 5636 |
| HSV1-UL30-2638 | + | ACGGGAUCCGGUCCUUG | 17 | 5637 |
| HSV1-UL30-2639 | + | GAUCCGGUCCUUGAUGG | 17 | 5638 |
| HSV1-UL30-2640 | + | AUCCGGUCCUUGAUGGA | 17 | 5639 |
| HSV1-UL30-2641 | + | GACGGGACCUGCGCGCG | 17 | 5640 |
| HSV1-UL30-2642 | + | CCUGCGCGCGGCGGGCC | 17 | 5641 |
| HSV1-UL30-2643 | + | UUGUAAUACACCGUCAG | 17 | 5642 |
| HSV1-UL30-2644 | + | CUUGUUGGUGUACGCGC | 17 | 5643 |
| HSV1-UL30-2645 | + | GUCUGCUCAGUUCGGCG | 17 | 5644 |
| HSV1-UL30-2646 | + | CUGCUCAGUUCGGCGGU | 17 | 5645 |
| HSV1-UL30-2647 | + | GCGGUGAGGACAAAGUC | 17 | 5646 |
| HSV1-UL30-2648 | + | GUCCUGGAUGUCCCUCU | 17 | 5647 |
| HSV1-UL30-2649 | + | GGGUCGGUGAUGCGCCG | 17 | 5648 |
| HSV1-UL30-2650 | + | UGCGCCGAUGGGCGUCU | 17 | 5649 |
| HSV1-UL30-2651 | + | CGCCGAUGGGCGUCUAC | 17 | 5650 |
| HSV1-UL30-2652 | + | CGUCUACGAGGACGGCC | 17 | 5651 |
| HSV1-UL30-2653 | + | CGAACGCCUGCAGUCCC | 17 | 5652 |
| HSV1-UL30-2654 | + | GCCUGCAGUCCCUCGGG | 17 | 5653 |
| HSV1-UL30-2655 | + | CCUGCAGUCCCUCGGGC | 17 | 5654 |
| HSV1-UL30-2656 | + | GCGCCAGCCACUCCUCC | 17 | 5655 |
| HSV1-UL30-2657 | + | CGCCAGCCACUCCUCCG | 17 | 5656 |
| HSV1-UL30-2658 | + | CUAACGCGGCGGCCGCU | 17 | 5657 |
| HSV1-UL30-2659 | + | UAACGCGGCGGCCGCUC | 17 | 5658 |
| HSV1-UL30-2660 | + | UAAAACAGCAGGUCGAC | 17 | 5659 |
| HSV1-UL30-2661 | + | GCAGGUCGACCAGGGCC | 17 | 5660 |
| HSV1-UL30-2662 | + | CAGGUCGACCAGGGCCC | 17 | 5661 |
| HSV1-UL30-2663 | + | CCAGAUCCACGCCCUUG | 17 | 5662 |
| HSV1-UL30-2664 | + | CGAUCAGCAGCAGCUUG | 17 | 5663 |
| HSV1-UL30-2665 | + | UGAACGUCUUUUCGCAC | 17 | 5664 |
| HSV1-UL30-2666 | + | UUUCGCACUCGAGUUUG | 17 | 5665 |
| HSV1-UL30-2667 | + | UUCGCACUCGAGUUUGA | 17 | 5666 |
| HSV1-UL30-2668 | + | UCGCACUCGAGUUUGAU | 17 | 5667 |
| HSV1-UL30-2669 | + | CGCACUCGAGUUUGAUG | 17 | 5668 |
| HSV1-UL30-2670 | + | UCGAGUUUGAUGGGGGG | 17 | 5669 |
| HSV1-UL30-2671 | + | GGGCAGAAACAGCGCGC | 17 | 5670 |
| HSV1-UL30-2672 | + | CCGUCAGCCCGGCGGCC | 17 | 5671 |
| HSV1-UL30-2673 | + | CGCGGCACAGCACAAAG | 17 | 5672 |
| HSV1-UL30-1554 | + | GCGGCACAGCACAAAGA | 17 | 4492 |
| HSV1-UL30-2675 | + | CCCCGUAGAUGAUGCGC | 17 | 5673 |
| HSV1-UL30-2676 | + | CCCGUAGAUGAUGCGCA | 17 | 5674 |
| HSV1-UL30-2677 | + | AGAUGAUGCGCAUGGAA | 17 | 5675 |
| HSV1-UL30-2678 | + | UGCGCAUGGAAUAGGGC | 17 | 5676 |
| HSV1-UL30-2679 | + | GCGCAUGGAAUAGGGCC | 17 | 5677 |
| HSV1-UL30-2680 | + | CGCAUGGAAUAGGGCCC | 17 | 5678 |
| HSV1-UL30-2681 | + | GCGCAUGUCGGCCGCCU | 17 | 5679 |
| HSV1-UL30-2682 | + | CGCAUGUCGGCCGCCUC | 17 | 5680 |
| HSV1-UL30-2683 | + | GCAUGUCGGCCGCCUCC | 17 | 5681 |
| HSV1-UL30-2684 | + | GCCUCCGGGAAAUCGGC | 17 | 5682 |
| HSV1-UL30-2685 | + | CCUCCGGGAAAUCGGCC | 17 | 5683 |
| HSV1-UL30-2686 | + | AAUCGGCCAGGAGCUGU | 17 | 5684 |
| HSV1-UL30-2687 | + | AAGGCCGCCCAGCGCGC | 17 | 5685 |
| HSV1-UL30-2688 | + | CGCGCGUGGACGUACUC | 17 | 5686 |
| HSV1-UL30-2689 | + | GGACGUACUCGCGGGUC | 17 | 5687 |
| HSV1-UL30-2690 | + | GCAACGUGCAGGCACGG | 17 | 5688 |
| HSV1-UL30-2691 | + | CAACGUGCAGGCACGGC | 17 | 5689 |
| HSV1-UL30-2692 | + | GUCCGUGCUGCACUCCC | 17 | 5690 |
| HSV1-UL30-2693 | + | UCCCGUGAACCCGUACA | 17 | 5691 |
| HSV1-UL30-2694 | + | GCGGCCUGCUGCUUGUC | 17 | 5692 |
| HSV1-UL30-2695 | + | CGGCCUGCUGCUUGUCC | 17 | 5693 |
| HSV1-UL30-2696 | + | CCAGGAGCACGGCCUCC | 17 | 5694 |
| HSV1-UL30-2697 | + | CAGGAGCACGGCCUCCU | 17 | 5695 |
| HSV1-UL30-2698 | + | GCCUCCUCGGGGCUGCU | 17 | 5696 |
| HSV1-UL30-2699 | + | CCUCCUCGGGGCUGCUC | 17 | 5697 |
| HSV1-UL30-2700 | + | CUCCUCGGGGCUGCUCU | 17 | 5698 |
| HSV1-UL30-2701 | + | UCCUCGGGGCUGCUCUG | 17 | 5699 |
| HSV1-UL30-2702 | + | GCUGCUCUGGGGAAUCC | 17 | 5700 |
| HSV1-UL30-2703 | + | CUCUGGGGAAUCCGCGA | 17 | 5701 |
| HSV1-UL30-2704 | + | GGAUCUGCUUUCGCAUG | 17 | 5702 |
| HSV1-UL30-2705 | + | AUGGCGAGCCAGUCCCG | 17 | 5703 |
| HSV1-UL30-2706 | + | UGGCGAGCCAGUCCCGC | 17 | 5704 |
| HSV1-UL30-2707 | + | GCGAGCCAGUCCCGCAG | 17 | 5705 |
| HSV1-UL30-2708 | + | AGUCCCGCAGGAGGAUG | 17 | 5706 |
| HSV1-UL30-2709 | + | UCCCGCAGGAGGAUGCU | 17 | 5707 |
| HSV1-UL30-2710 | + | CCCGCAGGAGGAUGCUG | 17 | 5708 |
| HSV1-UL30-2711 | + | AGGAGGCUCUCUCGCAC | 17 | 5709 |
| HSV1-UL30-2712 | + | CUCGCACGUGAGCCUUG | 17 | 5710 |
| HSV1-UL30-2713 | + | GCACGUGAGCCUUGACG | 17 | 5711 |
| HSV1-UL30-2714 | + | GCCACUGCGUCGGCCCU | 17 | 5712 |
| HSV1-UL30-2715 | + | CCACUGCGUCGGCCCUC | 17 | 5713 |
| HSV1-UL30-2716 | + | CACUGCGUCGGCCCUCA | 17 | 5714 |
| HSV1-UL30-2717 | + | CUGCGUCGGCCCUCAGG | 17 | 5715 |
| HSV1-UL30-2718 | + | CCCUCAGGGAGAGCGUG | 17 | 5716 |
| HSV1-UL30-2719 | + | GUGCUGAAGCACAGGUU | 17 | 5717 |
| HSV1-UL30-2720 | + | AAGCACAGGUUGUGGGC | 17 | 5718 |
| HSV1-UL30-2721 | + | UGUGGGCCUGGAUGAUG | 17 | 5719 |
| HSV1-UL30-2722 | + | GUGGGCCUGGAUGAUGC | 17 | 5720 |
| HSV1-UL30-2723 | + | GGUACAGGCUGGCAAAG | 17 | 5721 |
| HSV1-UL30-2724 | + | CAAAGUCGAACACCACC | 17 | 5722 |
| HSV1-UL30-2725 | + | AAAGUCGAACACCACCA | 17 | 5723 |
| HSV1-UL30-2726 | + | ACCACCACGGGGUUCAC | 17 | 5724 |
| HSV1-UL30-2727 | + | CGGGGUUCACGUGAAAC | 17 | 5725 |
| HSV1-UL30-2728 | + | GGGGUUCACGUGAAACC | 17 | 5726 |
| HSV1-UL30-2729 | + | UCACGUGAAACCCGGAA | 17 | 5727 |
| HSV1-UL30-2730 | + | CACGUGAAACCCGGAAG | 17 | 5728 |
| HSV1-UL30-2731 | + | AACCCGGAAGUGGGGUC | 17 | 5729 |
| HSV1-UL30-2732 | + | GGCCGCUCCUCGUCCUC | 17 | 5730 |
| HSV1-UL30-2733 | + | GUCCUCCCGGGCUGCGG | 17 | 5731 |
| HSV1-UL30-2734 | + | GGGCUGCGGCCGGACGC | 17 | 5732 |
| HSV1-UL30-2735 | + | GCGCCCCUAAAUCGCCC | 17 | 5733 |
| HSV1-UL30-2736 | + | CGCCCCUGGGUGUCCGG | 17 | 5734 |
| HSV1-UL30-2737 | + | AGCAGGCACGUAAAGAC | 17 | 5735 |
| HSV1-UL30-2738 | + | UGGCCGUCGUAGAUGGU | 17 | 5736 |
| HSV1-UL30-2739 | + | CGCCAAGCGCGCGACGG | 17 | 5737 |
| HSV1-UL30-2740 | + | CCAAGCGCGCGACGGCC | 17 | 5738 |
| HSV1-UL30-2741 | + | ACGGCCGAGAGCUCCAG | 17 | 5739 |
| HSV1-UL30-2742 | + | CGGCCGAGAGCUCCAGA | 17 | 5740 |
| HSV1-UL30-2743 | + | AACAGCUGGCCCACCAG | 17 | 5741 |
| HSV1-UL30-2744 | + | ACAGCUGGCCCACCAGC | 17 | 5742 |
| HSV1-UL30-2745 | + | CAGCUGGCCCACCAGCA | 17 | 5743 |
| HSV1-UL30-2746 | + | CGAUCACCCCGCGUUGC | 17 | 5744 |
| HSV1-UL30-2747 | + | GCCCGGCGGCGUAGUAG | 17 | 5745 |
| HSV1-UL30-2748 | + | CCCGGCGGCGUAGUAGG | 17 | 5746 |
| HSV1-UL30-2749 | + | CCGGCGGCGUAGUAGGC | 17 | 5747 |
| HSV1-UL30-2750 | + | UCCUUCUUCUUGUCCUU | 17 | 5748 |
| HSV1-UL30-2751 | + | CGGCUUCGGCCACGGCG | 17 | 5749 |
| HSV1-UL30-2752 | + | GGCGUUGAGCUUGUAGC | 17 | 5750 |
| HSV1-UL30-2753 | + | CGUUGAGCUUGUAGCUC | 17 | 5751 |
| HSV1-UL30-2754 | + | UUUAUCUUGCUGCGCUU | 17 | 5752 |
| HSV1-UL30-2755 | + | UUAUCUUGCUGCGCUUC | 17 | 5753 |
| HSV1-UL30-2756 | + | UGGCCUAUGUCCCACAC | 17 | 5754 |
| HSV1-UL30-2757 | + | AUGCGGCCGUACCCGUC | 17 | 5755 |
| HSV1-UL30-2758 | + | UGCGGCCGUACCCGUCC | 17 | 5756 |
| HSV1-UL30-2759 | + | GCGGCCGUACCCGUCCA | 17 | 5757 |
| HSV1-UL30-2760 | + | CGGCCGUACCCGUCCAG | 17 | 5758 |
| HSV1-UL30-2761 | + | CCGUCAGCUUGGCCAGC | 17 | 5759 |
| HSV1-UL30-2762 | + | UCAGCUUGGCCAGCAAG | 17 | 5760 |
| HSV1-UL30-2763 | + | CCAGCAAGAAGGGCCAG | 17 | 5761 |
| HSV1-UL30-2764 | + | UGAUGUUGUACCCGGUC | 17 | 5762 |
| HSV1-UL30-2765 | + | UGUACCCGGUCACGAAC | 17 | 5763 |
| HSV1-UL30-2766 | + | GUACCCGGUCACGAACU | 17 | 5764 |
| HSV1-UL30-2767 | + | GGGCCGUACUGUUUCAC | 17 | 5765 |
| HSV1-UL30-2768 | + | ACUGUUUCACAAGGGUC | 17 | 5766 |
| HSV1-UL30-2769 | + | UGAAGGCCAACAGCAUC | 17 | 5767 |
| HSV1-UL30-2770 | + | GCAUCUCGAAUUCGCUG | 17 | 5768 |
| HSV1-UL30-2771 | + | AAUUCGCUGUCGAAUUC | 17 | 5769 |
| HSV1-UL30-2772 | + | UGUCGAAUUCCAGAACC | 17 | 5770 |
| HSV1-UL30-2773 | + | AUUCCAGAACCACGGGC | 17 | 5771 |
| HSV1-UL30-2774 | + | GCCGCCAGCUCGUUCAG | 17 | 5772 |
| HSV1-UL30-2775 | + | CCGCCAGCUCGUUCAGG | 17 | 5773 |
| HSV1-UL30-2776 | + | GCUCGUUCAGGUGGGAU | 17 | 5774 |
| HSV1-UL30-2777 | + | CUCGUUCAGGUGGGAUU | 17 | 5775 |
| HSV1-UL30-2778 | + | UCGUUCAGGUGGGAUUC | 17 | 5776 |
| HSV1-UL30-2779 | + | CGUUCAGGUGGGAUUCG | 17 | 5777 |
| HSV1-UL30-2780 | + | GGGAUUCGGGGAGGUCG | 17 | 5778 |
| HSV1-UL30-2781 | + | GGAUUCGGGGAGGUCGC | 17 | 5779 |
| HSV1-UL30-2782 | + | CGGGGAGGUCGCAGGAA | 17 | 5780 |
| HSV1-UL30-2783 | + | GAGGUCGCAGGAACCGA | 17 | 5781 |
| HSV1-UL30-2784 | + | CAGGAACCGAGCGAAAA | 17 | 5782 |
| HSV1-UL30-1694 | + | AGGAACCGAGCGAAAAC | 17 | 4632 |
| HSV1-UL30-2786 | + | GAACCGAGCGAAAACAG | 17 | 5783 |
| HSV1-UL30-2787 | + | AACAGGAGGACGUGCUC | 17 | 5784 |
| HSV1-UL30-2788 | + | CGUGCUCCAGGGCGGUG | 17 | 5785 |
| HSV1-UL30-2789 | + | CGGUGGUGGACAGGUCG | 17 | 5786 |
| HSV1-UL30-2790 | + | ACAGGUCGUAGAGCAGA | 17 | 5787 |
| HSV1-UL30-2791 | + | UAGAGCAGACAGGAUAU | 17 | 5788 |
| HSV1-UL30-2792 | + | CUGGAUGACCAGGUCCU | 17 | 5789 |
| HSV1-UL30-2793 | + | CUCCGGGUGCCCGGCCA | 17 | 5790 |
| HSV1-UL30-2794 | + | UCCGGGUGCCCGGCCAC | 17 | 5791 |
| HSV1-UL30-2795 | + | CCGCCUUGCAUUCGAUA | 17 | 5792 |
| HSV1-UL30-2796 | + | AUUCGAUAUCGAAGCAC | 17 | 5793 |
| HSV1-UL30-2797 | + | AGUUAAACUCGACGUCG | 17 | 5794 |
| HSV1-UL30-2798 | + | CGACGUCGCUGGAUGUC | 17 | 5795 |
| HSV1-UL30-2799 | + | GGAUGUCCCGAAGGCCA | 17 | 5796 |
| HSV1-UL30-2800 | + | GAUGUCCCGAAGGCCAU | 17 | 5797 |
| HSV1-UL30-2801 | + | GCCAUCGGGGCCGCCGG | 17 | 5798 |
| HSV1-UL30-2802 | + | UGUUGUUCCGGCCCGGU | 17 | 5799 |
| HSV1-UL30-2803 | + | GUUUGAGACGGUACCAG | 17 | 5800 |
| HSV1-UL30-2804 | + | GGUACCAGCCGAAGGUG | 17 | 5801 |
| HSV1-UL30-2805 | + | AGCCGAAGGUGACGAAC | 17 | 5802 |
| HSV1-UL30-2806 | + | GCCGAAGGUGACGAACC | 17 | 5803 |
| HSV1-UL30-2807 | + | ACGAACCCGGGGUUGUC | 17 | 5804 |
| HSV1-UL30-2808 | + | ACCCGGGGUUGUCCAGG | 17 | 5805 |
| HSV1-UL30-2809 | + | GGGUUGUCCAGGAUGAA | 17 | 5806 |
| HSV1-UL30-2810 | + | CUCGUACUUCUUGAUGG | 17 | 5807 |
| HSV1-UL30-2811 | + | ACUUCUUGAUGGCCGGG | 17 | 5808 |
| HSV1-UL30-2812 | + | GACAGCACACGCCCGCU | 17 | 5809 |
| HSV1-UL30-2813 | + | ACGUAGACGCGGUAAAA | 17 | 5810 |
| HSV1-UL30-2814 | + | AGACGCGGUAAAACAGA | 17 | 5811 |
| HSV1-UL30-2815 | + | GACGCGGUAAAACAGAG | 17 | 5812 |
| HSV1-UL30-2816 | + | GCUCCACCACCUCCGCC | 17 | 5813 |
| HSV1-UL30-2817 | + | CCGCCUCGAAGUGGUCC | 17 | 5814 |
| HSV1-UL30-2818 | + | CGCCUCGAAGUGGUCCG | 17 | 5815 |
| HSV1-UL30-2819 | + | UGGUCCGCGGAGAUGCC | 17 | 5816 |
| HSV1-UL30-2820 | + | GGUCCGCGGAGAUGCCG | 17 | 5817 |
| HSV1-UL30-2821 | + | GCCGCGGAACGACGCGC | 17 | 5818 |
| HSV1-UL30-1747 | + | CCGCGGAACGACGCGCC | 17 | 4685 |
| HSV1-UL30-1748 | + | CGCGGAACGACGCGCCC | 17 | 4686 |
| HSV1-UL30-2824 | + | GCGCCCGGGGACUCGCG | 17 | 5819 |
| HSV1-UL30-2825 | + | CCGCGGCCAUGCGCUCG | 17 | 5820 |
| HSV1-UL30-2826 | + | GCGCUCGCAGAGAUCUC | 17 | 5821 |
| HSV1-UL30-2827 | + | CGCUCGCAGAGAUCUCG | 17 | 5822 |
| HSV1-UL30-2828 | + | UGCCGCGUGCCGUAAAC | 17 | 5823 |
| HSV1-UL30-2829 | + | GGCCACCCGGUGGCCUU | 17 | 5824 |
| HSV1-UL30-2830 | + | GCCACCCGGUGGCCUUC | 17 | 5825 |
| HSV1-UL30-2831 | + | CCUUCCGGAGUCAGGCC | 17 | 5826 |
| HSV1-UL30-2832 | + | CUUCCGGAGUCAGGCCC | 17 | 5827 |

Table 2F provides exemplary targeting domains for knocking out the UL30 gene selected according to the first tier parameters. The targeting domains are selected based on location within first 500 bp of the coding sequence of the UL30 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 2F

| | | | Target | |
|---|---|---|---|---|
| 1st Tier | DNA | | Site | |
| gRNA Name | Strand | Targeting Domain | Length | Seq ID |
| HSV1- | − | ACUAUAGCGAAUGCGAUGAA | 20 | 5828 |
| UL30-419 | | | | |
| HSV1- | − | AUAGCGAAUGCGAUGAA | 17 | 5829 |
| UL30-420 | | | | |

Table 2G provides targeting domains for knocking out the UL30 gene selected according to the second tier parameters. The targeting domains are selected based on location within the coding sequence (but downstream of the first 500 bp) of the UL30 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the RNAs is 0-50 bp.

TABLE 2G

| 2nd Tier | DNA | | Target Site | |
|---|---|---|---|---|
| gRNA Name | Strand | Targeting Domain | Length | Seq ID |
| HSV1-UL30-1857 | − | CAUGGUGAACAUCGACAUGU | 20 | 4907 |
| HSV1-UL30-2834 | − | GUGAUCGGCGAGUACUGCAU | 20 | 5830 |
| HSV1-UL30-2835 | − | UUAUUCUGCCGGACACCCAG | 20 | 5831 |
| HSV1-UL30-2836 | − | CAUGCGAAAGCAGAUCCGCU | 20 | 5832 |
| HSV1-UL30-2837 | − | GCGGCCUUCGAACAGCUCCU | 20 | 5833 |
| HSV1-UL30-2139 | − | GGUGAACAUCGACAUGU | 17 | 5172 |
| HSV1-UL30-2839 | − | AUCGGCGAGUACUGCAU | 17 | 5834 |
| HSV1-UL30-2840 | − | UUCUGCCGGACACCCAG | 17 | 5835 |
| HSV1-UL30-2841 | − | GCGAAAGCAGAUCCGCU | 17 | 5836 |
| HSV1-UL30-2842 | − | GCCUUCGAACAGCUCCU | 17 | 5837 |
| HSV1-UL30-2525 | + | CUGGCCGCCAGCUCGUUCAG | 20 | 5531 |
| HSV1-UL30-2774 | + | GCCGCCAGCUCGUUCAG | 17 | 5772 |

Table 3A provides exemplary targeting domains for knocking out the UL48 gene selected according to first tier parameters. The targeting domains are selected based on location within the first 500 bp of the coding sequence of the UL48 gene and orthogonality against the human genome. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp

TABLE 3A

| 1st Tier | | | Target Site | Seq |
|---|---|---|---|---|
| gRNA Name | DNA Strand | Targeting Domain | Length | ID |
| HSV1-UL48-8 | − | CGGGGGUCCCAAAAACACCC | 20 | 5838 |
| HSV1-UL48-9 | − | GGGUCCCAAAAACACCCCGG | 20 | 5839 |
| HSV1-UL48-10 | − | GGCCCCCCCGCUGUACGCAA | 20 | 5840 |
| HSV1-UL48-11 | − | GCCCCCCCGCUGUACGCAAC | 20 | 5841 |
| HSV1-UL48-12 | − | CCCCCCCGCUGUACGCAACG | 20 | 5842 |
| HSV1-UL48-13 | − | CGCAACGGGGCGCCUGAGCC | 20 | 5843 |
| HSV1-UL48-14 | − | UAACCGUCUCCUCGACGACU | 20 | 5844 |
| HSV1-UL48-15 | − | AACCGUCUCCUCGACGACUU | 20 | 5845 |
| HSV1-UL48-16 | − | CGACGACUUGGGCUUUAGCG | 20 | 5846 |
| HSV1-UL48-17 | − | GACGACUUGGGCUUUAGCGC | 20 | 5847 |
| HSV1-UL48-126 | − | GGGUCCCAAAAACACCC | 17 | 5848 |
| HSV1-UL48-127 | − | UCCCAAAAACACCCCGG | 17 | 5849 |
| HSV1-UL48-128 | − | CCCCCCGCUGUACGCAA | 17 | 5850 |
| HSV1-UL48-129 | − | CCCCCGCUGUACGCAAC | 17 | 5851 |
| HSV1-UL48-130 | − | CCCCGCUGUACGCAACG | 17 | 5852 |
| HSV1-UL48-131 | − | AACGGGGCGCCUGAGCC | 17 | 5853 |
| HSV1-UL48-132 | − | CCGUCUCCUCGACGACU | 17 | 5854 |
| HSV1-UL48-133 | − | CGUCUCCUCGACGACUU | 17 | 5855 |
| HSV1-UL48-134 | − | CGACUUGGGCUUUAGCG | 17 | 5856 |
| HSV1-UL48-135 | − | GACUUGGGCUUUAGCGC | 17 | 5857 |
| HSV1-UL48-19 | − | CAUGCUCGAUACCUGGAACG | 20 | 5858 |
| HSV1-UL48-20 | − | CUGGAACGAGGAUCUGUUUU | 20 | 5859 |
| HSV1-UL48-21 | − | CGACCAACGCCGACCUGUAC | 20 | 5860 |
| HSV1-UL48-22 | − | GACCAACGCCGACCUGUACC | 20 | 5861 |
| HSV1-UL48-23 | − | AUCAACGCUGCCCAGCGAUG | 20 | 5862 |
| HSV1-UL48-24 | − | AACGCUGCCCAGCGAUGUGG | 20 | 5863 |
| HSV1-UL48-25 | − | UGCCCAGCGAUGUGGUGGAA | 20 | 5864 |
| HSV1-UL48-137 | − | GCUCGAUACCUGGAACG | 17 | 5865 |
| HSV1-UL48-138 | − | GAACGAGGAUCUGUUUU | 17 | 5866 |
| HSV1-UL48-139 | − | CCAACGCCGACCUGUAC | 17 | 5867 |
| HSV1-UL48-140 | − | CAACGCCGACCUGUACC | 17 | 5868 |
| HSV1-UL48-141 | − | AACGCUGCCCAGCGAUG | 17 | 5869 |
| HSV1-UL48-142 | − | GCUGCCCAGCGAUGUGG | 17 | 5870 |
| HSV1-UL48-143 | − | CCAGCGAUGUGGUGGAA | 17 | 5871 |
| HSV1-UL48-31 | − | CAUUCGCGCCCACGGCGACG | 20 | 5872 |
| HSV1-UL48-32 | − | CGUGGCCUUCCCUACGCUUC | 20 | 5873 |
| HSV1-UL48-33 | − | ACGCUUCCGGCCACCCGCGA | 20 | 5874 |
| HSV1-UL48-34 | − | CCGGCCACCCGCGACGGCCU | 20 | 5875 |
| HSV1-UL48-35 | − | CGGCCACCCGCGACGGCCUC | 20 | 5876 |
| HSV1-UL48-36 | − | GUUUCUUCCACGCCGAGCUA | 20 | 5877 |
| HSV1-UL48-37 | − | UUUCUUCCACGCCGAGCUAC | 20 | 5878 |
| HSV1-UL48-38 | − | UCCACGCCGAGCUACGGGCG | 20 | 5879 |
| HSV1-UL48-39 | − | CCACGCCGAGCUACGGGCGC | 20 | 5880 |
| HSV1-UL48-149 | − | UCGCGCCCACGGCGACG | 17 | 5881 |
| HSV1-UL48-150 | − | GGCCUUCCCUACGCUUC | 17 | 5882 |
| HSV1-UL48-151 | − | CUUCCGGCCACCCGCGA | 17 | 5883 |
| HSV1-UL48-152 | − | GCCACCCGCGACGGCCU | 17 | 5884 |
| HSV1-UL48-153 | − | CCACCCGCGACGGCCUC | 17 | 5885 |
| HSV1-UL48-154 | − | UCUUCCACGCCGAGCUA | 17 | 5886 |
| HSV1-UL48-155 | − | CUUCCACGCCGAGCUAC | 17 | 5887 |
| HSV1-UL48-156 | − | ACGCCGAGCUACGGGCG | 17 | 5888 |
| HSV1-UL48-157 | − | CGCCGAGCUACGGGCGC | 17 | 5889 |
| HSV1-UL48-41 | + | CUCCUCCCGCGCCCGUAGCU | 20 | 5890 |
| HSV1-UL48-43 | + | GCGCUUCGUAGUAGAGCCCG | 20 | 5891 |
| HSV1-UL48-45 | + | GUAGAGCCCGAGGCCGUCGC | 20 | 5892 |
| HSV1-UL48-159 | + | CUCCCGCGCCCGUAGCU | 17 | 5893 |
| HSV1-UL48-161 | + | CUUCGUAGUAGAGCCCG | 17 | 5894 |
| HSV1-UL48-163 | + | GAGCCCGAGGCCGUCGC | 17 | 5895 |
| HSV1-UL48-48 | + | UCGCGGGUGGCCGGAAGCGU | 20 | 5896 |
| HSV1-UL48-166 | + | CGGGUGGCCGGAAGCGU | 17 | 5897 |
| HSV1-UL48-51 | + | UAGGGAAGGCCACGUCGCCG | 20 | 5898 |
| HSV1-UL48-52 | + | AGGGAAGGCCACGUCGCCGU | 20 | 5899 |
| HSV1-UL48-53 | + | CGUGGGCGCGAAUGUCGAUU | 20 | 5900 |
| HSV1-UL48-54 | + | GUGGGCGCGAAUGUCGAUUU | 20 | 5901 |
| HSV1-UL48-169 | + | GGAAGGCCACGUCGCCG | 17 | 5902 |
| HSV1-UL48-170 | + | GAAGGCCACGUCGCCGU | 17 | 5903 |
| HSV1-UL48-171 | + | GGGCGCGAAUGUCGAUU | 17 | 5904 |
| HSV1-UL48-172 | + | GGCGCGAAUGUCGAUUU | 17 | 5905 |
| HSV1-UL48-58 | + | CCCCCAUUCCACCACAUCGC | 20 | 5906 |
| HSV1-UL48-176 | + | CCAUUCCACCACAUCGC | 17 | 5907 |
| HSV1-UL48-64 | + | CUCCCGGUACAGGUCGGCGU | 20 | 5908 |
| HSV1-UL48-65 | + | CGGUACAGGUCGGCGUUGGU | 20 | 5909 |
| HSV1-UL48-66 | + | CGAAAACAGAUCCUCGUUCC | 20 | 5910 |
| HSV1-UL48-67 | + | CUCGUUCCAGGUAUCGAGCA | 20 | 5911 |
| HSV1-UL48-68 | + | AUCGAGCAUGGUACAUAGCG | 20 | 5912 |
| HSV1-UL48-69 | + | UCGAGCAUGGUACAUAGCGC | 20 | 5913 |
| HSV1-UL48-70 | + | CGAGCAUGGUACAUAGCGCG | 20 | 5914 |
| HSV1-UL48-71 | + | CGCUAAAGCCCAAGUCGUCG | 20 | 5915 |
| HSV1-UL48-182 | + | CCGGUACAGGUCGGCGU | 17 | 5916 |
| HSV1-UL48-183 | + | UACAGGUCGGCGUUGGU | 17 | 5917 |
| HSV1-UL48-184 | + | AAACAGAUCCUCGUUCC | 17 | 5918 |
| HSV1-UL48-185 | + | GUUCCAGGUAUCGAGCA | 17 | 5919 |
| HSV1-UL48-186 | + | GAGCAUGGUACAUAGCG | 17 | 5920 |
| HSV1-UL48-187 | + | AGCAUGGUACAUAGCGC | 17 | 5921 |
| HSV1-UL48-188 | + | GCAUGGUACAUAGCGCG | 17 | 5922 |
| HSV1-UL48-189 | + | UAAAGCCCAAGUCGUCG | 17 | 5923 |
| HSV1-UL48-73 | + | CGUCGAGGAGACGGUUAAAG | 20 | 5924 |
| HSV1-UL48-74 | + | GUCGAGGAGACGGUUAAAGA | 20 | 5925 |
| HSV1-UL48-191 | + | CGAGGAGACGGUUAAAG | 17 | 5926 |
| HSV1-UL48-192 | + | GAGGAGACGGUUAAAGA | 17 | 5927 |
| HSV1-UL48-76 | + | GAGACGGUUAAAGAGGGCGG | 20 | 5928 |
| HSV1-UL48-77 | + | AGACGGUUAAAGAGGGCGGC | 20 | 5929 |
| HSV1-UL48-78 | + | GACGGUUAAAGAGGGCGGCG | 20 | 5930 |
| HSV1-UL48-79 | + | ACGGUUAAAGAGGGCGGCGG | 20 | 5931 |
| HSV1-UL48-80 | + | CGGUUAAAGAGGGCGGCGGG | 20 | 5932 |
| HSV1-UL48-194 | + | ACGGUUAAAGAGGGCGG | 17 | 5933 |
| HSV1-UL48-195 | + | CGGUUAAAGAGGGCGGC | 17 | 5934 |
| HSV1-UL48-196 | + | GGUUAAAGAGGGCGGCG | 17 | 5935 |
| HSV1-UL48-197 | + | GUUAAAGAGGGCGGCGG | 17 | 5936 |
| HSV1-UL48-198 | + | UUAAAGAGGGCGGCGGG | 17 | 5937 |
| HSV1-UL48-97 | + | GCGCCCCGUUGCGUACAGCG | 20 | 5938 |
| HSV1-UL48-98 | + | CGCCCCGUUGCGUACAGCGG | 20 | 5939 |
| HSV1-UL48-99 | + | GCCCCGUUGCGUACAGCGGG | 20 | 5940 |
| HSV1-UL48-100 | + | CCCCGUUGCGUACAGCGGGG | 20 | 5941 |
| HSV1-UL48-215 | + | CCCCGUUGCGUACAGCG | 17 | 5942 |
| HSV1-UL48-216 | + | CCCGUUGCGUACAGCGG | 17 | 5943 |
| HSV1-UL48-217 | + | CCGUUGCGUACAGCGGG | 17 | 5944 |
| HSV1-UL48-218 | + | CGUUGCGUACAGCGGGG | 17 | 5945 |
| HSV1-UL48-102 | + | CGUACAGCGGGGGGGCCGCC | 20 | 5946 |
| HSV1-UL48-103 | + | GUACAGCGGGGGGGCCGCCG | 20 | 5947 |
| HSV1-UL48-104 | + | GGGGGCCGCCGGGGUGUUUU | 20 | 5948 |
| HSV1-UL48-105 | + | GGGGCCGCCGGGGUGUUUUU | 20 | 5949 |
| HSV1-UL48-220 | + | ACAGCGGGGGGGCCGCC | 17 | 5950 |
| HSV1-UL48-221 | + | CAGCGGGGGGGCCGCCG | 17 | 5951 |
| HSV1-UL48-222 | + | GGCCGCCGGGGUGUUUU | 17 | 5952 |
| HSV1-UL48-223 | + | GCCGCCGGGGUGUUUUU | 17 | 5953 |
| HSV1-UL48-107 | + | GUGUUUUUGGGACCCCCGGC | 20 | 5954 |
| HSV1-UL48-225 | + | UUUUUGGGACCCCCGGC | 17 | 5955 |
| HSV1-UL48-108 | + | UGUUUUUGGGACCCCCGGCC | 20 | 5956 |
| HSV1-UL48-226 | + | UUUUGGGACCCCCGGCC | 17 | 5957 |
| HSV1-UL48-118 | + | CAAACAGCUCGUCGACCAAG | 20 | 5958 |
| HSV1-UL48-236 | + | ACAGCUCGUCGACCAAG | 17 | 5959 |

Table 3B provides exemplary targeting domains for knocking out the UL48 gene selected according to the second tier parameters. The targeting domains are selected based on location within the first 500 bp of the coding sequence of the UL48 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 3B

| 2nd Tier | | | Target Site | Seq |
|---|---|---|---|---|
| gRNA Name | DNA Strand | Targeting Domain | Length | ID |
| HSV1-UL48-1 | − | GCUGUUUGCCGACAUGAACG | 20 | 5960 |
| HSV1-UL48-2 | − | UUUGCCGACAUGAACGCGGA | 20 | 5961 |
| HSV1-UL48-3 | − | UUCGCCACCGCCCCCCCGCC | 20 | 5962 |
| HSV1-UL48-4 | − | CCACCGCCCCCCCGCCCGGC | 20 | 5963 |
| HSV1-UL48-5 | − | CACCGCCCCCCCGCCCGGCC | 20 | 5964 |
| HSV1-UL48-6 | − | ACCGCCCCCCCGCCCGGCCG | 20 | 5965 |
| HSV1-UL48-7 | − | CCGCCCCCCCGCCCGGCCGG | 20 | 5966 |
| HSV1-UL48-18 | − | UAUGUACCAUGCUCGAUACC | 20 | 5967 |
| HSV1-UL48-26 | − | GCCCAGCGAUGUGGUGGAAU | 20 | 5968 |
| HSV1-UL48-27 | − | CCCAGCGAUGUGGUGGAAUG | 20 | 5969 |
| HSV1-UL48-28 | − | CCAGCGAUGUGGUGGAAUGG | 20 | 5970 |
| HSV1-UL48-29 | − | CAGCGAUGUGGUGGAAUGGG | 20 | 5971 |
| HSV1-UL48-30 | − | CAAAUCGACAUUCGCGCCCA | 20 | 5972 |
| HSV1-UL48-40 | − | CGCCGAGCUACGGGCGCGGG | 20 | 5973 |
| HSV1-UL48-119 | − | GUUUGCCGACAUGAACG | 17 | 5974 |
| HSV1-UL48-120 | − | GCCGACAUGAACGCGGA | 17 | 5975 |
| HSV1-UL48-121 | − | GCCACCGCCCCCCCGCC | 17 | 5976 |
| HSV1-UL48-122 | − | CCGCCCCCCCGCCCGGC | 17 | 5977 |
| HSV1-UL48-123 | − | CGCCCCCCCGCCCGGCC | 17 | 5978 |
| HSV1-UL48-124 | − | GCCCCCCCGCCCGGCCG | 17 | 5979 |
| HSV1-UL48-125 | − | CCCCCCCGCCCGGCCGG | 17 | 5980 |
| HSV1-UL48-136 | − | GUACCAUGCUCGAUACC | 17 | 5981 |
| HSV1-UL48-144 | − | CAGCGAUGUGGUGGAAU | 17 | 5982 |
| HSV1-UL48-145 | − | AGCGAUGUGGUGGAAUG | 17 | 5983 |
| HSV1-UL48-146 | − | GCGAUGUGGUGGAAUGG | 17 | 5984 |
| HSV1-UL48-147 | − | CGAUGUGGUGGAAUGGG | 17 | 5985 |
| HSV1-UL48-148 | − | AUCGACAUUCGCGCCCA | 17 | 5986 |
| HSV1-UL48-158 | − | CGAGCUACGGGCGCGGG | 17 | 5987 |
| HSV1-UL48-42 | + | CCCGCGCCCGUAGCUCGGCG | 20 | 5988 |
| HSV1-UL48-44 | + | AGUAGAGCCCGAGGCCGUCG | 20 | 5989 |
| HSV1-UL48-46 | + | GAGCCCGAGGCCGUCGCGGG | 20 | 5990 |
| HSV1-UL48-47 | + | CCGAGGCCGUCGCGGGUGGC | 20 | 5991 |
| HSV1-UL48-49 | + | CGCGGGUGGCCGGAAGCGUA | 20 | 5992 |
| HSV1-UL48-50 | + | GGUGGCCGGAAGCGUAGGGA | 20 | 5993 |
| HSV1-UL48-55 | + | AAUGUCGAUUUGGGUGCGUU | 20 | 5994 |
| HSV1-UL48-56 | + | AUGUCGAUUUGGGUGCGUUC | 20 | 5995 |
| HSV1-UL48-57 | + | UGUCGAUUUGGGUGCGUUCG | 20 | 5996 |
| HSV1-UL48-59 | + | CCCCAUUCCACCACAUCGCU | 20 | 5997 |
| HSV1-UL48-60 | + | CAUCGCUGGGCAGCGUUGAU | 20 | 5998 |
| HSV1-UL48-61 | + | UUGAUAGGAAUUUACACUCC | 20 | 5999 |
| HSV1-UL48-62 | + | GGAAUUUACACUCCCGGUAC | 20 | 6000 |
| HSV1-UL48-63 | + | UUUACACUCCCGGUACAGGU | 20 | 6001 |
| HSV1-UL48-72 | + | AGCCCAAGUCGUCGAGGAGA | 20 | 6002 |
| HSV1-UL48-75 | + | GAGGAGACGGUUAAAGAGGG | 20 | 6003 |
| HSV1-UL48-81 | + | GGUUAAAGAGGGCGGCGGGG | 20 | 6004 |
| HSV1-UL48-82 | + | AAAGAGGGCGGCGGGGGGGA | 20 | 6005 |
| HSV1-UL48-83 | + | AAGAGGGCGGCGGGGGGGAC | 20 | 6006 |
| HSV1-UL48-84 | + | GGCGGCGGGGGGGACGGGCA | 20 | 6007 |
| HSV1-UL48-85 | + | GCGGCGGGGGGGACGGGCAU | 20 | 6008 |
| HSV1-UL48-86 | + | GCGGGGGGGACGGGCAUGGG | 20 | 6009 |
| HSV1-UL48-87 | + | CGGGGGGGACGGGCAUGGGU | 20 | 6010 |
| HSV1-UL48-88 | + | GGGGGGGACGGGCAUGGGUG | 20 | 6011 |
| HSV1-UL48-89 | + | GGGGACGGGCAUGGGUGGGG | 20 | 6012 |
| HSV1-UL48-90 | + | GGGACGGGCAUGGGUGGGGA | 20 | 6013 |
| HSV1-UL48-91 | + | UGGGUGGGGAGGGCAUGAGC | 20 | 6014 |
| HSV1-UL48-92 | + | GGGUGGGGAGGGCAUGAGCU | 20 | 6015 |
| HSV1-UL48-93 | + | GGGAGGGCAUGAGCUGGGCC | 20 | 6016 |
| HSV1-UL48-94 | + | GCAUGAGCUGGGCCUGGCUC | 20 | 6017 |
| HSV1-UL48-95 | + | AGGCGCCCCGUUGCGUACAG | 20 | 6018 |
| HSV1-UL48-96 | + | GGCGCCCCGUUGCGUACAGC | 20 | 6019 |
| HSV1-UL48-101 | + | GCGUACAGCGGGGGGGCCGC | 20 | 6020 |
| HSV1-UL48-106 | + | CGGGGUGUUUUUGGGACCCC | 20 | 6021 |
| HSV1-UL48-109 | + | UUUUGGGACCCCCGGCCGGG | 20 | 6022 |
| HSV1-UL48-110 | + | UUUGGGACCCCCGGCCGGGC | 20 | 6023 |
| HSV1-UL48-111 | + | UUGGGACCCCCGGCCGGGCG | 20 | 6024 |
| HSV1-UL48-112 | + | UGGGACCCCCGGCCGGGCGG | 20 | 6025 |
| HSV1-UL48-113 | + | GGGACCCCCGGCCGGGCGGG | 20 | 6026 |
| HSV1-UL48-114 | + | GGACCCCCGGCCGGGCGGGG | 20 | 6027 |
| HSV1-UL48-115 | + | CCCCCGGCCGGGCGGGGGGG | 20 | 6028 |
| HSV1-UL48-116 | + | CCGGCCGGGCGGGGGGGCGG | 20 | 6029 |
| HSV1-UL48-117 | + | AGCGCCGUCCGCGUUCAUGU | 20 | 6030 |
| HSV1-UL48-160 | + | GCGCCCGUAGCUCGGCG | 17 | 6031 |
| HSV1-UL48-162 | + | AGAGCCCGAGGCCGUCG | 17 | 6032 |
| HSV1-UL48-164 | + | CCCGAGGCCGUCGCGGG | 17 | 6033 |
| HSV1-UL48-165 | + | AGGCCGUCGCGGGUGGC | 17 | 6034 |
| HSV1-UL48-167 | + | GGGUGGCCGGAAGCGUA | 17 | 6035 |
| HSV1-UL48-168 | + | GGCCGGAAGCGUAGGGA | 17 | 6036 |
| HSV1-UL48-173 | + | GUCGAUUUGGGUGCGUU | 17 | 6037 |
| HSV1-UL48-174 | + | UCGAUUUGGGUGCGUUC | 17 | 6038 |
| HSV1-UL48-175 | + | CGAUUUGGGUGCGUUCG | 17 | 6039 |
| HSV1-UL48-177 | + | CAUUCCACCACAUCGCU | 17 | 6040 |
| HSV1-UL48-178 | + | CGCUGGGCAGCGUUGAU | 17 | 6041 |
| HSV1-UL48-179 | + | AUAGGAAUUUACACUCC | 17 | 6042 |
| HSV1-UL48-180 | + | AUUUACACUCCCGGUAC | 17 | 6043 |
| HSV1-UL48-181 | + | ACACUCCCGGUACAGGU | 17 | 6044 |
| HSV1-UL48-190 | + | CCAAGUCGUCGAGGAGA | 17 | 6045 |
| HSV1-UL48-193 | + | GAGACGGUUAAAGAGGG | 17 | 6046 |
| HSV1-UL48-199 | + | UAAAGAGGGCGGCGGGG | 17 | 6047 |
| HSV1-UL48-200 | + | GAGGGCGGCGGGGGGGA | 17 | 6048 |
| HSV1-UL48-201 | + | AGGGCGGCGGGGGGGAC | 17 | 6049 |
| HSV1-UL48-202 | + | GGCGGGGGGGACGGGCA | 17 | 6050 |
| HSV1-UL48-203 | + | GCGGGGGGGACGGGCAU | 17 | 6051 |
| HSV1-UL48-204 | + | GGGGGGACGGGCAUGGG | 17 | 6052 |
| HSV1-UL48-205 | + | GGGGGACGGGCAUGGGU | 17 | 6053 |
| HSV1-UL48-206 | + | GGGGACGGGCAUGGGUG | 17 | 6054 |
| HSV1-UL48-207 | + | GACGGGCAUGGGUGGGG | 17 | 6055 |
| HSV1-UL48-208 | + | ACGGGCAUGGGUGGGGA | 17 | 6056 |
| HSV1-UL48-209 | + | GUGGGGAGGGCAUGAGC | 17 | 6057 |
| HSV1-UL48-210 | + | UGGGGAGGGCAUGAGCU | 17 | 6058 |
| HSV1-UL48-211 | + | AGGGCAUGAGCUGGGCC | 17 | 6059 |
| HSV1-UL48-212 | + | UGAGCUGGGCCUGGCUC | 17 | 6060 |
| HSV1-UL48-213 | + | CGCCCCGUUGCGUACAG | 17 | 6061 |
| HSV1-UL48-214 | + | GCCCCGUUGCGUACAGC | 17 | 6062 |
| HSV1-UL48-219 | + | UACAGCGGGGGGGCCGC | 17 | 6063 |
| HSV1-UL48-224 | + | GGUGUUUUUGGGACCCC | 17 | 6064 |
| HSV1-UL48-227 | + | UGGGACCCCCGGCCGGG | 17 | 6065 |
| HSV1-UL48-228 | + | GGGACCCCCGGCCGGGC | 17 | 6066 |
| HSV1-UL48-229 | + | GGACCCCCGGCCGGGCG | 17 | 6067 |
| HSV1-UL48-230 | + | GACCCCCGGCCGGGCGG | 17 | 6068 |
| HSV1-UL48-231 | + | ACCCCCGGCCGGGCGGG | 17 | 6069 |
| HSV1-UL48-232 | + | CCCCCGGCCGGGCGGGG | 17 | 6070 |
| HSV1-UL48-233 | + | CCGGCCGGGCGGGGGGG | 17 | 6071 |
| HSV1-UL48-234 | + | GCCGGGCGGGGGGGCGG | 17 | 6072 |
| HSV1-UL48-235 | + | GCCGUCCGCGUUCAUGU | 17 | 6073 |

Table 3C provides exemplary targeting domains for knocking out the UL48 gene selected according to the third tier parameters. The targeting domains are selected based on location within the coding sequence, but downstream of the first 500 bp of the UL48 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 3C

| 3rd Tier | | | Target Site | Seq |
|---|---|---|---|---|
| gRNA Name | DNA Strand | Targeting Domain | Length | ID |
| HSV1-UL48- | − | CGUGUUGGCCAACUUCUGCU | 20 | 6074 |
| 1126 | | | | |
| HSV1-UL48-237 | − | ACUUCUGCUCGGCCCUGUAC | 20 | 6075 |
| HSV1-UL48-238 | − | GGUACCUGCGCGCCAGCGUC | 20 | 6076 |
| HSV1-UL48-239 | − | CGUCCGGCAGCUGCACCGCC | 20 | 6077 |
| HSV1-UL48-240 | − | CACCGCCAGGCGCACAUGCG | 20 | 6078 |
| HSV1-UL48-241 | − | GCGCGGACGCGAUCGCGACC | 20 | 6079 |
| HSV1-UL48-242 | − | CGCGGACGCGAUCGCGACCU | 20 | 6080 |
| HSV1-UL48-243 | − | AAUGCUGCGCGCCACGAUCG | 20 | 6081 |
| HSV1-UL48-244 | − | UGCGCGCCACGAUCGCGGAC | 20 | 6082 |
| HSV1-UL48-245 | − | CUACCGAGAGACCGCUCGUC | 20 | 6083 |
| HSV1-UL48-246 | − | UUUUGACCCGCGAGAUCCUA | 20 | 6084 |
| HSV1-UL48-247 | − | UUUGACCCGCGAGAUCCUAU | 20 | 6085 |
| HSV1-UL48-248 | − | CGUACGCCGAGCAGAUGAUG | 20 | 6086 |
| HSV1-UL48-249 | − | UGACUGCCUCUGUUGCGACC | 20 | 6087 |
| HSV1-UL48-250 | − | UCUGUUGCGACCUGGAGAGC | 20 | 6088 |
| HSV1-UL48-251 | − | CCUGGAGAGCUGGCGUCAGU | 20 | 6089 |
| HSV1-UL48-252 | − | GGAGAGCUGGCGUCAGUUGG | 20 | 6090 |
| HSV1-UL48-253 | − | GAGAGCUGGCGUCAGUUGGC | 20 | 6091 |
| HSV1-UL48-254 | − | CAGCCCUUCAUGUUCGUCAA | 20 | 6092 |
| HSV1-UL48-255 | − | UCAACGGAGCGCUCACCGUC | 20 | 6093 |
| HSV1-UL48-256 | − | CAACGGAGCGCUCACCGUCC | 20 | 6094 |
| HSV1-UL48-257 | − | AACGGAGCGCUCACCGUCCG | 20 | 6095 |
| HSV1-UL48-258 | − | CGUCCGGGGAGUGCCAAUCG | 20 | 6096 |
| HSV1-UL48-259 | − | GAGUGCCAAUCGAGGCCCGC | 20 | 6097 |
| HSV1-UL48-260 | − | CAAUCGAGGCCCGCCGGCUG | 20 | 6098 |
| HSV1-UL48-261 | − | AAUCGAGGCCCGCCGGCUGC | 20 | 6099 |
| HSV1-UL48-262 | − | CGAGCACCUUAACCUCCCGC | 20 | 6100 |
| HSV1-UL48-263 | − | CCUCCCGCUGGUGCGCAGCG | 20 | 6101 |
| HSV1-UL48-264 | − | GCUGGUGCGCAGCGCGGCUA | 20 | 6102 |
| HSV1-UL48-265 | − | GGUGCGCAGCGCGGCUACGG | 20 | 6103 |
| HSV1-UL48-266 | − | AGCGCGGCUACGGAGGAGCC | 20 | 6104 |
| HSV1-UL48-267 | − | GCGCGGCUACGGAGGAGCCA | 20 | 6105 |
| HSV1-UL48-268 | − | CGCGGCUACGGAGGAGCCAG | 20 | 6106 |
| HSV1-UL48-269 | − | ACGACCCCUCCCACCCUGCA | 20 | 6107 |
| HSV1-UL48-270 | − | UCCCACCCUGCAUGGCAACC | 20 | 6108 |
| HSV1-UL48-271 | − | GGCAACCAGGCCCGCGCCUC | 20 | 6109 |
| HSV1-UL48-272 | − | GCAACCAGGCCCGCGCCUCU | 20 | 6110 |
| HSV1-UL48-273 | − | CCGCGCCUCUGGGUACUUUA | 20 | 6111 |
| HSV1-UL48-274 | − | GGUACUUUAUGGUGUUGAUU | 20 | 6112 |
| HSV1-UL48-275 | − | GUACUUUAUGGUGUUGAUUC | 20 | 6113 |
| HSV1-UL48-276 | − | GGUGUUGAUUCGGGCGAAGU | 20 | 6114 |
| HSV1-UL48-277 | − | CUUCACGACCUCGCCCUCCG | 20 | 6115 |
| HSV1-UL48-278 | − | CACGACCUCGCCCUCCGAGG | 20 | 6116 |
| HSV1-UL48-279 | − | CGCCCUCCGAGGCGGUCAUG | 20 | 6117 |
| HSV1-UL48-280 | − | GCCCUCCGAGGCGGUCAUGC | 20 | 6118 |
| HSV1-UL48-281 | − | GCGCGUACGAAAAACAAUUA | 20 | 6119 |
| HSV1-UL48-282 | − | CGCGUACGAAAAACAAUUAC | 20 | 6120 |
| HSV1-UL48-283 | − | CAAUUACGGGUCUACCAUCG | 20 | 6121 |
| HSV1-UL48-284 | − | AAUUACGGGUCUACCAUCGA | 20 | 6122 |
| HSV1-UL48-285 | − | CGAGGGCCUGCUCGAUCUCC | 20 | 6123 |
| HSV1-UL48-286 | − | GGACGACGACGCCCCCGAAG | 20 | 6124 |
| HSV1-UL48-287 | − | CGACGACGCCCCCGAAGAGG | 20 | 6125 |
| HSV1-UL48-288 | − | GACGACGCCCCCGAAGAGGC | 20 | 6126 |
| HSV1-UL48-289 | − | ACGACGCCCCCGAAGAGGCG | 20 | 6127 |
| HSV1-UL48-290 | − | CGCCCCCGAAGAGGCGGGGC | 20 | 6128 |
| HSV1-UL48-291 | − | CCCCGAAGAGGCGGGGCUGG | 20 | 6129 |
| HSV1-UL48-292 | − | GCGCCUGUCCUUUCUCCCCG | 20 | 6130 |
| HSV1-UL48-293 | − | CGCCUGUCCUUUCUCCCCGC | 20 | 6131 |
| HSV1-UL48-294 | − | ACACACGCGCAGACUGUCGA | 20 | 6132 |
| HSV1-UL48-295 | − | CCCCCCGACCGAUGUCAGCC | 20 | 6133 |
| HSV1-UL48-296 | − | CCCCCGACCGAUGUCAGCCU | 20 | 6134 |
| HSV1-UL48-297 | − | CCCCGACCGAUGUCAGCCUG | 20 | 6135 |
| HSV1-UL48-298 | − | CCCGACCGAUGUCAGCCUGG | 20 | 6136 |
| HSV1-UL48-299 | − | GGGGACGAGCUCCACUUAGA | 20 | 6137 |
| HSV1-UL48-300 | − | CGAGCUCCACUUAGACGGCG | 20 | 6138 |
| HSV1-UL48-301 | − | CCACUUAGACGGCGAGGACG | 20 | 6139 |
| HSV1-UL48-302 | − | AGACGGCGAGGACGUGGCGA | 20 | 6140 |
| HSV1-UL48-303 | − | CGCGCUAGACGAUUUCGAUC | 20 | 6141 |
| HSV1-UL48-304 | − | CGAUUUCGAUCUGGACAUGU | 20 | 6142 |
| HSV1-UL48-305 | − | GAUUUCGAUCUGGACAUGUU | 20 | 6143 |
| HSV1-UL48-306 | − | AUUUCGAUCUGGACAUGUUG | 20 | 6144 |
| HSV1-UL48-307 | − | UUUCGAUCUGGACAUGUUGG | 20 | 6145 |
| HSV1-UL48-308 | − | GAUCUGGACAUGUUGGGGGA | 20 | 6146 |
| HSV1-UL48-309 | − | AUCUGGACAUGUUGGGGGAC | 20 | 6147 |
| HSV1-UL48-310 | − | UCUGGACAUGUUGGGGGACG | 20 | 6148 |
| HSV1-UL48-311 | − | GUUGGGGGACGGGGAUUCCC | 20 | 6149 |
| HSV1-UL48-312 | − | UUGGGGGACGGGGAUUCCCC | 20 | 6150 |
| HSV1-UL48-313 | − | UGGGGGACGGGGAUUCCCCG | 20 | 6151 |
| HSV1-UL48-314 | − | GGACGGGGAUUCCCCGGGGC | 20 | 6152 |
| HSV1-UL48-315 | − | GACGGGGAUUCCCCGGGGCC | 20 | 6153 |
| HSV1-UL48-316 | − | CCCCACGACUCCGCCCCCUA | 20 | 6154 |
| HSV1-UL48-317 | − | CUCCGCCCCCUACGGCGCUC | 20 | 6155 |
| HSV1-UL48-318 | − | CCCCUACGGCGCUCUGGAUA | 20 | 6156 |
| HSV1-UL48-319 | − | CAGAUGUUUACCGAUGCCCU | 20 | 6157 |
| HSV1-UL48-320 | − | GCCCUUGGAAUUGACGAGUA | 20 | 6158 |
| HSV1-UL48-321 | − | CUUGGAAUUGACGAGUACGG | 20 | 6159 |
| HSV1-UL48-322 | − | UUGGAAUUGACGAGUACGGU | 20 | 6160 |
| HSV1-UL48-323 | + | CACCGUACUCGUCAAUUCCA | 20 | 6161 |
| HSV1-UL48-324 | + | ACCGUACUCGUCAAUUCCAA | 20 | 6162 |
| HSV1-UL48-325 | + | CUCGUCAAUUCCAAGGGCAU | 20 | 6163 |
| HSV1-UL48-326 | + | CAUCUGCUCAAACUCGAAGU | 20 | 6164 |
| HSV1-UL48-327 | + | GGCCAUAUCCAGAGCGCCGU | 20 | 6165 |
| HSV1-UL48-328 | + | GCCAUAUCCAGAGCGCCGUA | 20 | 6166 |
| HSV1-UL48-329 | + | CCAUAUCCAGAGCGCCGUAG | 20 | 6167 |
| HSV1-UL48-330 | + | CAUAUCCAGAGCGCCGUAGG | 20 | 6168 |
| HSV1-UL48-331 | + | AUCCAGAGCGCCGUAGGGGG | 20 | 6169 |
| HSV1-UL48-332 | + | CGCCGUAGGGGGCGGAGUCG | 20 | 6170 |
| HSV1-UL48-333 | + | GCCGUAGGGGGCGGAGUCGU | 20 | 6171 |
| HSV1-UL48-334 | + | CCGUAGGGGGCGGAGUCGUG | 20 | 6172 |
| HSV1-UL48-335 | + | CGUAGGGGGCGGAGUCGUGG | 20 | 6173 |
| HSV1-UL48-336 | + | GUAGGGGGCGGAGUCGUGGG | 20 | 6174 |
| HSV1-UL48-337 | + | GAGUCGUGGGGGGUAAAUCC | 20 | 6175 |
| HSV1-UL48-338 | + | UGGGGGGUAAAUCCCGGCCC | 20 | 6176 |
| HSV1-UL48-339 | + | GGGGGGUAAAUCCCGGCCCC | 20 | 6177 |
| HSV1-UL48-340 | + | GGGGGUAAAUCCCGGCCCCG | 20 | 6178 |
| HSV1-UL48-341 | + | AUCGAAAUCGUCUAGCGCGU | 20 | 6179 |
| HSV1-UL48-342 | + | CCACGUCCUCGCCGUCUAAG | 20 | 6180 |
| HSV1-UL48-343 | + | CUAAGUGGAGCUCGUCCCCC | 20 | 6181 |
| HSV1-UL48-344 | + | CUCGUCCCCCAGGCUGACAU | 20 | 6182 |
| HSV1-UL48-345 | + | UCCCCCAGGCUGACAUCGGU | 20 | 6183 |
| HSV1-UL48-346 | + | CCCCCAGGCUGACAUCGGUC | 20 | 6184 |
| HSV1-UL48-347 | + | CCCCAGGCUGACAUCGGUCG | 20 | 6185 |
| HSV1-UL48-348 | + | CCCAGGCUGACAUCGGUCGG | 20 | 6186 |
| HSV1-UL48-349 | + | CCAGGCUGACAUCGGUCGGG | 20 | 6187 |
| HSV1-UL48-350 | + | CAGGCUGACAUCGGUCGGGG | 20 | 6188 |
| HSV1-UL48-351 | + | CAGUCUGCGCGUGUGUCCCG | 20 | 6189 |
| HSV1-UL48-352 | + | AGUCUGCGCGUGUGUCCCGC | 20 | 6190 |
| HSV1-UL48-353 | + | GUCUGCGCGUGUGUCCCGCG | 20 | 6191 |
| HSV1-UL48-354 | + | CGUGUGUCCCGCGGGGAGAA | 20 | 6192 |
| HSV1-UL48-355 | + | GUCCCGCGGGGAGAAAGGAC | 20 | 6193 |
| HSV1-UL48-356 | + | GCGGGGAGAAAGGACAGGCG | 20 | 6194 |
| HSV1-UL48-357 | + | AGCCGCCAGCCCCGCCUCUU | 20 | 6195 |
| HSV1-UL48-358 | + | GCCGCCAGCCCCGCCUCUUC | 20 | 6196 |
| HSV1-UL48-359 | + | CCGCCAGCCCCGCCUCUUCG | 20 | 6197 |
| HSV1-UL48-360 | + | CGCCAGCCCCGCCUCUUCGG | 20 | 6198 |
| HSV1-UL48-361 | + | UCUUCGGGGGCGUCGUCGUC | 20 | 6199 |
| HSV1-UL48-362 | + | CUUCGGGGGCGUCGUCGUCC | 20 | 6200 |
| HSV1-UL48-363 | + | CGUCGUCCGGGAGAUCGAGC | 20 | 6201 |
| HSV1-UL48-364 | + | GAGAUCGAGCAGGCCCUCGA | 20 | 6202 |
| HSV1-UL48-365 | + | AAUUGUUUUUCGUACGCGCG | 20 | 6203 |
| HSV1-UL48-366 | + | GUGUUCCCGCAUGACCGCCU | 20 | 6204 |
| HSV1-UL48-367 | + | UUCCCGCAUGACCGCCUCGG | 20 | 6205 |
| HSV1-UL48-368 | + | UCCCGCAUGACCGCCUCGGA | 20 | 6206 |
| HSV1-UL48-369 | + | CAUGACCGCCUCGGAGGGCG | 20 | 6207 |
| HSV1-UL48-370 | + | GGAGGGCGAGGUCGUGAAGC | 20 | 6208 |
| HSV1-UL48-371 | + | CAACACCAUAAAGUACCCAG | 20 | 6209 |
| HSV1-UL48-372 | + | CCAUAAAGUACCCAGAGGCG | 20 | 6210 |
| HSV1-UL48-373 | + | CAUAAAGUACCCAGAGGCGC | 20 | 6211 |
| HSV1-UL48-374 | + | AGUACCCAGAGGCGCGGGCC | 20 | 6212 |
| HSV1-UL48-375 | + | CGCGGGCCUGGUUGCCAUGC | 20 | 6213 |
| HSV1-UL48-376 | + | GCGGGCCUGGUUGCCAUGCA | 20 | 6214 |
| HSV1-UL48-377 | + | GGCCUGGUUGCCAUGCAGGG | 20 | 6215 |
| HSV1-UL48-378 | + | GCCUGGUUGCCAUGCAGGGU | 20 | 6216 |
| HSV1-UL48-379 | + | UGGUUGCCAUGCAGGGUGGG | 20 | 6217 |
| HSV1-UL48-380 | + | GGUUGCCAUGCAGGGUGGGA | 20 | 6218 |
| HSV1-UL48-381 | + | GUUGCCAUGCAGGGUGGGAG | 20 | 6219 |
| HSV1-UL48-382 | + | AGGGUGGGAGGGGUCGUCAA | 20 | 6220 |
| HSV1-UL48-383 | + | GGGGUCGUCAACGGCGCCCC | 20 | 6221 |
| HSV1-UL48-384 | + | GUAGCCGCGCUGCGCACCAG | 20 | 6222 |
| HSV1-UL48-385 | + | UAGCCGCGCUGCGCACCAGC | 20 | 6223 |
| HSV1-UL48-386 | + | CCGCGCUGCGCACCAGCGGG | 20 | 6224 |
| HSV1-UL48-387 | + | UGCGCACCAGCGGGAGGUUA | 20 | 6225 |
| HSV1-UL48-388 | + | GGUUAAGGUGCUCGCGAAUG | 20 | 6226 |
| HSV1-UL48-389 | + | UGUGGUUUAGCUCCCGCAGC | 20 | 6227 |
| HSV1-UL48-390 | + | GGUUUAGCUCCCGCAGCCGG | 20 | 6228 |
| HSV1-UL48-391 | + | GUUUAGCUCCCGCAGCCGGC | 20 | 6229 |
| HSV1-UL48-392 | + | CGCAGCCGGCGGGCCUCGAU | 20 | 6230 |
| HSV1-UL48-393 | + | GGGCCUCGAUUGGCACUCCC | 20 | 6231 |
| HSV1-UL48-394 | + | CUCGAUUGGCACUCCCCGGA | 20 | 6232 |
| HSV1-UL48-395 | + | CGCUCCGUUGACGAACAUGA | 20 | 6233 |
| HSV1-UL48-396 | + | GCUCCGUUGACGAACAUGAA | 20 | 6234 |
| HSV1-UL48-397 | + | CGUUGACGAACAUGAAGGGC | 20 | 6235 |
| HSV1-UL48-398 | + | CCAACUGACGCCAGCUCUCC | 20 | 6236 |
| HSV1-UL48-399 | + | AGCUCUCCAGGUCGCAACAG | 20 | 6237 |
| HSV1-UL48-400 | + | CGCAACAGAGGCAGUCAAAC | 20 | 6238 |
| HSV1-UL48-401 | + | ACAGAGGCAGUCAAACAGGU | 20 | 6239 |
| HSV1-UL48-402 | + | CAGAGGCAGUCAAACAGGUC | 20 | 6240 |
| HSV1-UL48-403 | + | GUCGGGCCGCAUCAUCUGCU | 20 | 6241 |
| HSV1-UL48-404 | + | CAUCAUCUGCUCGGCGUACG | 20 | 6242 |
| HSV1-UL48-405 | + | GCUCGGCGUACGCGGCCCAU | 20 | 6243 |
| HSV1-UL48-406 | + | ACGCGGCCCAUAGGAUCUCG | 20 | 6244 |
| HSV1-UL48-407 | + | CGCGGCCCAUAGGAUCUCGC | 20 | 6245 |
| HSV1-UL48-408 | + | CAAAACACGCGCCAGACGAG | 20 | 6246 |
| HSV1-UL48-409 | + | GCGCCAGACGAGCGGUCUCU | 20 | 6247 |
| HSV1-UL48-410 | + | GUAGUACCUGUCCGCGAUCG | 20 | 6248 |
| HSV1-UL48-411 | + | UGGCGCGCAGCAUUUCUCCC | 20 | 6249 |
| HSV1-UL48-412 | + | CGCGUCCGCGCAUGUGCGCC | 20 | 6250 |
| HSV1-UL48-413 | + | GUCCGCGCAUGUGCGCCUGG | 20 | 6251 |
| HSV1-UL48-414 | + | GCGCCUGGCGGUGCAGCUGC | 20 | 6252 |
| HSV1-UL48-415 | + | GCGGUGCAGCUGCCGGACGC | 20 | 6253 |
| HSV1-UL48-416 | + | GCUGCCGGACGCUGGCGCGC | 20 | 6254 |
| HSV1-UL48-417 | + | GGACGCUGGCGCGCAGGUAC | 20 | 6255 |
| HSV1-UL48-418 | + | UGGCGCGCAGGUACCGGUAC | 20 | 6256 |
| HSV1-UL48-419 | + | GGCGCGCAGGUACCGGUACA | 20 | 6257 |
| HSV1-UL48-420 | + | GUACAGGGCCGAGCAGAAGU | 20 | 6258 |
| HSV1-UL48-421 | + | CGAGCAGAAGUUGGCCAACA | 20 | 6259 |
| HSV1-UL48-422 | − | GUUGGCCAACUUCUGCU | 17 | 6260 |
| HSV1-UL48-423 | − | UCUGCUCGGCCCUGUAC | 17 | 6261 |
| HSV1-UL48-424 | − | ACCUGCGCGCCAGCGUC | 17 | 6262 |
| HSV1-UL48-425 | − | CCGGCAGCUGCACCGCC | 17 | 6263 |
| HSV1-UL48-426 | − | CGCCAGGCGCACAUGCG | 17 | 6264 |
| HSV1-UL48-427 | − | CGGACGCGAUCGCGACC | 17 | 6265 |
| HSV1-UL48-428 | − | GGACGCGAUCGCGACCU | 17 | 6266 |
| HSV1-UL48-429 | − | GCUGCGCGCCACGAUCG | 17 | 6267 |
| HSV1-UL48-430 | − | GCGCCACGAUCGCGGAC | 17 | 6268 |
| HSV1-UL48-431 | − | CCGAGAGACCGCUCGUC | 17 | 6269 |
| HSV1-UL48-432 | − | UGACCCGCGAGAUCCUA | 17 | 6270 |
| HSV1-UL48-433 | − | GACCCGCGAGAUCCUAU | 17 | 6271 |
| HSV1-UL48-434 | − | ACGCCGAGCAGAUGAUG | 17 | 6272 |
| HSV1-UL48-435 | − | CUGCCUCUGUUGCGACC | 17 | 6273 |
| HSV1-UL48-436 | − | GUUGCGACCUGGAGAGC | 17 | 6274 |
| HSV1-UL48-437 | − | GGAGAGCUGGCGUCAGU | 17 | 6275 |
| HSV1-UL48-438 | − | GAGCUGGCGUCAGUUGG | 17 | 6276 |
| HSV1-UL48-439 | − | AGCUGGCGUCAGUUGGC | 17 | 6277 |
| HSV1-UL48-440 | − | CCCUUCAUGUUCGUCAA | 17 | 6278 |
| HSV1-UL48-441 | − | ACGGAGCGCUCACCGUC | 17 | 6279 |
| HSV1-UL48-442 | − | CGGAGCGCUCACCGUCC | 17 | 6280 |
| HSV1-UL48-443 | − | GGAGCGCUCACCGUCCG | 17 | 6281 |
| HSV1-UL48-444 | − | CCGGGGAGUGCCAAUCG | 17 | 6282 |
| HSV1-UL48-445 | − | UGCCAAUCGAGGCCCGC | 17 | 6283 |
| HSV1-UL48-446 | − | UCGAGGCCCGCCGGCUG | 17 | 6284 |
| HSV1-UL48-447 | − | CGAGGCCCGCCGGCUGC | 17 | 6285 |
| HSV1-UL48-448 | − | GCACCUUAACCUCCCGC | 17 | 6286 |
| HSV1-UL48-449 | − | CCCGCUGGUGCGCAGCG | 17 | 6287 |
| HSV1-UL48-450 | − | GGUGCGCAGCGCGGCUA | 17 | 6288 |
| HSV1-UL48-451 | − | GCGCAGCGCGGCUACGG | 17 | 6289 |
| HSV1-UL48-452 | − | GCGGCUACGGAGGAGCC | 17 | 6290 |
| HSV1-UL48-453 | − | CGGCUACGGAGGAGCCA | 17 | 6291 |
| HSV1-UL48-454 | − | GGCUACGGAGGAGCCAG | 17 | 6292 |
| HSV1-UL48-455 | − | ACCCCUCCCACCCUGCA | 17 | 6293 |
| HSV1-UL48-456 | − | CACCCUGCAUGGCAACC | 17 | 6294 |
| HSV1-UL48-457 | − | AACCAGGCCCGCGCCUC | 17 | 6295 |
| HSV1-UL48-458 | − | ACCAGGCCCGCGCCUCU | 17 | 6296 |
| HSV1-UL48-459 | − | CGCCUCUGGGUACUUUA | 17 | 6297 |
| HSV1-UL48-460 | − | ACUUUAUGGUGUUGAUU | 17 | 6298 |
| HSV1-UL48-461 | − | CUUUAUGGUGUUGAUUC | 17 | 6299 |
| HSV1-UL48-462 | − | GUUGAUUCGGGCGAAGU | 17 | 6300 |
| HSV1-UL48-463 | − | CACGACCUCGCCCUCCG | 17 | 6301 |
| HSV1-UL48-464 | − | GACCUCGCCCUCCGAGG | 17 | 6302 |
| HSV1-UL48-465 | − | CCUCCGAGGCGGUCAUG | 17 | 6303 |
| HSV1-UL48-466 | − | CUCCGAGGCGGUCAUGC | 17 | 6304 |
| HSV1-UL48-467 | − | CGUACGAAAAACAAUUA | 17 | 6305 |
| HSV1-UL48-468 | − | GUACGAAAAACAAUUAC | 17 | 6306 |
| HSV1-UL48-469 | − | UUACGGGUCUACCAUCG | 17 | 6307 |
| HSV1-UL48-470 | − | UACGGGUCUACCAUCGA | 17 | 6308 |
| HSV1-UL48-471 | − | GGGCCUGCUCGAUCUCC | 17 | 6309 |
| HSV1-UL48-472 | − | CGACGACGCCCCCGAAG | 17 | 6310 |
| HSV1-UL48-473 | − | CGACGCCCCCGAAGAGG | 17 | 6311 |
| HSV1-UL48-474 | − | GACGCCCCCGAAGAGGC | 17 | 6312 |
| HSV1-UL48-475 | − | ACGCCCCCGAAGAGGCG | 17 | 6313 |
| HSV1-UL48-476 | − | CCCCGAAGAGGCGGGGC | 17 | 6314 |
| HSV1-UL48-477 | − | CGAAGAGGCGGGGCUGG | 17 | 6315 |
| HSV1-UL48-478 | − | CCUGUCCUUUCUCCCCG | 17 | 6316 |
| HSV1-UL48-479 | − | CUGUCCUUUCUCCCCGC | 17 | 6317 |
| HSV1-UL48-480 | − | CACGCGCAGACUGUCGA | 17 | 6318 |
| HSV1-UL48-481 | − | CCCGACCGAUGUCAGCC | 17 | 6319 |
| HSV1-UL48-482 | − | CCGACCGAUGUCAGCCU | 17 | 6320 |
| HSV1-UL48-483 | − | CGACCGAUGUCAGCCUG | 17 | 6321 |
| HSV1-UL48-484 | − | GACCGAUGUCAGCCUGG | 17 | 6322 |
| HSV1-UL48-485 | − | GACGAGCUCCACUUAGA | 17 | 6323 |
| HSV1-UL48-486 | − | GCUCCACUUAGACGGCG | 17 | 6324 |
| HSV1-UL48-487 | − | CUUAGACGGCGAGGACG | 17 | 6325 |
| HSV1-UL48-488 | − | CGGCGAGGACGUGGCGA | 17 | 6326 |
| HSV1-UL48-489 | − | GCUAGACGAUUUCGAUC | 17 | 6327 |
| HSV1-UL48-490 | − | UUUCGAUCUGGACAUGU | 17 | 6328 |
| HSV1-UL48-491 | − | UUCGAUCUGGACAUGUU | 17 | 6329 |
| HSV1-UL48-492 | − | UCGAUCUGGACAUGUUG | 17 | 6330 |
| HSV1-UL48-493 | − | CGAUCUGGACAUGUUGG | 17 | 6331 |
| HSV1-UL48-494 | − | CUGGACAUGUUGGGGGA | 17 | 6332 |
| HSV1-UL48-495 | − | UGGACAUGUUGGGGGAC | 17 | 6333 |
| HSV1-UL48-496 | − | GGACAUGUUGGGGGACG | 17 | 6334 |
| HSV1-UL48-497 | − | GGGGGACGGGGAUUCCC | 17 | 6335 |
| HSV1-UL48-498 | − | GGGGACGGGGAUUCCCC | 17 | 6336 |
| HSV1-UL48-499 | − | GGGACGGGGAUUCCCCG | 17 | 6337 |
| HSV1-UL48-500 | − | CGGGGAUUCCCCGGGGC | 17 | 6338 |
| HSV1-UL48-501 | − | GGGGAUUCCCCGGGGCC | 17 | 6339 |
| HSV1-UL48-502 | − | CACGACUCCGCCCCCUA | 17 | 6340 |
| HSV1-UL48-503 | − | CGCCCCCUACGGCGCUC | 17 | 6341 |
| HSV1-UL48-504 | − | CUACGGCGCUCUGGAUA | 17 | 6342 |
| HSV1-UL48-505 | − | AUGUUUACCGAUGCCCU | 17 | 6343 |
| HSV1-UL48-506 | − | CUUGGAAUUGACGAGUA | 17 | 6344 |
| HSV1-UL48-507 | − | GGAAUUGACGAGUACGG | 17 | 6345 |
| HSV1-UL48-508 | − | GAAUUGACGAGUACGGU | 17 | 6346 |
| HSV1-UL48-509 | + | CGUACUCGUCAAUUCCA | 17 | 6347 |
| HSV1-UL48-510 | + | GUACUCGUCAAUUCCAA | 17 | 6348 |
| HSV1-UL48-511 | + | GUCAAUUCCAAGGGCAU | 17 | 6349 |
| HSV1-UL48-512 | + | CUGCUCAAACUCGAAGU | 17 | 6350 |
| HSV1-UL48-513 | + | CAUAUCCAGAGCGCCGU | 17 | 6351 |
| HSV1-UL48-514 | + | AUAUCCAGAGCGCCGUA | 17 | 6352 |
| HSV1-UL48-515 | + | UAUCCAGAGCGCCGUAG | 17 | 6353 |
| HSV1-UL48-516 | + | AUCCAGAGCGCCGUAGG | 17 | 6354 |
| HSV1-UL48-517 | + | CAGAGCGCCGUAGGGGG | 17 | 6355 |
| HSV1-UL48-518 | + | CGUAGGGGGCGGAGUCG | 17 | 6356 |
| HSV1-UL48-519 | + | GUAGGGGGCGGAGUCGU | 17 | 6357 |
| HSV1-UL48-520 | + | UAGGGGGCGGAGUCGUG | 17 | 6358 |
| HSV1-UL48-521 | + | AGGGGGCGGAGUCGUGG | 17 | 6359 |
| HSV1-UL48-522 | + | GGGGGCGGAGUCGUGGG | 17 | 6360 |
| HSV1-UL48-523 | + | UCGUGGGGGGUAAAUCC | 17 | 6361 |
| HSV1-UL48-524 | + | GGGGUAAAUCCCGGCCC | 17 | 6362 |
| HSV1-UL48-525 | + | GGGUAAAUCCCGGCCCC | 17 | 6363 |
| HSV1-UL48-526 | + | GGUAAAUCCCGGCCCCG | 17 | 6364 |
| HSV1-UL48-527 | + | GAAAUCGUCUAGCGCGU | 17 | 6365 |
| HSV1-UL48-528 | + | CGUCCUCGCCGUCUAAG | 17 | 6366 |
| HSV1-UL48-529 | + | AGUGGAGCUCGUCCCCC | 17 | 6367 |
| HSV1-UL48-530 | + | GUCCCCCAGGCUGACAU | 17 | 6368 |
| HSV1-UL48-531 | + | CCCAGGCUGACAUCGGU | 17 | 6369 |
| HSV1-UL48-532 | + | CCAGGCUGACAUCGGUC | 17 | 6370 |
| HSV1-UL48-533 | + | CAGGCUGACAUCGGUCG | 17 | 6371 |
| HSV1-UL48-534 | + | AGGCUGACAUCGGUCGG | 17 | 6372 |
| HSV1-UL48-535 | + | GGCUGACAUCGGUCGGG | 17 | 6373 |
| HSV1-UL48-536 | + | GCUGACAUCGGUCGGGG | 17 | 6374 |
| HSV1-UL48-537 | + | UCUGCGCGUGUGUCCCG | 17 | 6375 |
| HSV1-UL48-538 | + | CUGCGCGUGUGUCCCGC | 17 | 6376 |
| HSV1-UL48-539 | + | UGCGCGUGUGUCCCGCG | 17 | 6377 |
| HSV1-UL48-540 | + | GUGUCCCGCGGGGAGAA | 17 | 6378 |
| HSV1-UL48-541 | + | CCGCGGGGAGAAAGGAC | 17 | 6379 |
| HSV1-UL48-542 | + | GGGAGAAAGGACAGGCG | 17 | 6380 |
| HSV1-UL48-543 | + | CGCCAGCCCCGCCUCUU | 17 | 6381 |
| HSV1-UL48-544 | + | GCCAGCCCCGCCUCUUC | 17 | 6382 |
| HSV1-UL48-545 | + | CCAGCCCCGCCUCUUCG | 17 | 6383 |
| HSV1-UL48-546 | + | CAGCCCCGCCUCUUCGG | 17 | 6384 |
| HSV1-UL48-547 | + | UCGGGGGCGUCGUCGUC | 17 | 6385 |
| HSV1-UL48-548 | + | CGGGGGCGUCGUCGUCC | 17 | 6386 |
| HSV1-UL48-549 | + | CGUCCGGGAGAUCGAGC | 17 | 6387 |
| HSV1-UL48-550 | + | AUCGAGCAGGCCCUCGA | 17 | 6388 |
| HSV1-UL48-551 | + | UGUUUUUCGUACGCGCG | 17 | 6389 |
| HSV1-UL48-552 | + | UUCCCGCAUGACCGCCU | 17 | 6390 |
| HSV1-UL48-553 | + | CCGCAUGACCGCCUCGG | 17 | 6391 |
| HSV1-UL48-554 | + | CGCAUGACCGCCUCGGA | 17 | 6392 |
| HSV1-UL48-555 | + | GACCGCCUCGGAGGGCG | 17 | 6393 |
| HSV1-UL48-556 | + | GGGCGAGGUCGUGAAGC | 17 | 6394 |
| HSV1-UL48-557 | + | CACCAUAAAGUACCCAG | 17 | 6395 |
| HSV1-UL48-558 | + | UAAAGUACCCAGAGGCG | 17 | 6396 |
| HSV1-UL48-559 | + | AAAGUACCCAGAGGCGC | 17 | 6397 |
| HSV1-UL48-560 | + | ACCCAGAGGCGCGGGCC | 17 | 6398 |
| HSV1-UL48-561 | + | GGGCCUGGUUGCCAUGC | 17 | 6399 |
| HSV1-UL48-562 | + | GGCCUGGUUGCCAUGCA | 17 | 6400 |
| HSV1-UL48-563 | + | CUGGUUGCCAUGCAGGG | 17 | 6401 |
| HSV1-UL48-564 | + | UGGUUGCCAUGCAGGGU | 17 | 6402 |
| HSV1-UL48-565 | + | UUGCCAUGCAGGGUGGG | 17 | 6403 |
| HSV1-UL48-566 | + | UGCCAUGCAGGGUGGGA | 17 | 6404 |
| HSV1-UL48-567 | + | GCCAUGCAGGGUGGGAG | 17 | 6405 |
| HSV1-UL48-568 | + | GUGGGAGGGGUCGUCAA | 17 | 6406 |
| HSV1-UL48-569 | + | GUCGUCAACGGCGCCCC | 17 | 6407 |
| HSV1-UL48-570 | + | GCCGCGCUGCGCACCAG | 17 | 6408 |
| HSV1-UL48-571 | + | CCGCGCUGCGCACCAGC | 17 | 6409 |
| HSV1-UL48-572 | + | CGCUGCGCACCAGCGGG | 17 | 6410 |
| HSV1-UL48-573 | + | GCACCAGCGGGAGGUUA | 17 | 6411 |
| HSV1-UL48-574 | + | UAAGGUGCUCGCGAAUG | 17 | 6412 |
| HSV1-UL48-575 | + | GGUUUAGCUCCCGCAGC | 17 | 6413 |
| HSV1-UL48-576 | + | UUAGCUCCCGCAGCCGG | 17 | 6414 |
| HSV1-UL48-577 | + | UAGCUCCCGCAGCCGGC | 17 | 6415 |
| HSV1-UL48-578 | + | AGCCGGCGGGCCUCGAU | 17 | 6416 |
| HSV1-UL48-579 | + | CCUCGAUUGGCACUCCC | 17 | 6417 |
| HSV1-UL48-580 | + | GAUUGGCACUCCCCGGA | 17 | 6418 |
| HSV1-UL48-581 | + | UCCGUUGACGAACAUGA | 17 | 6419 |
| HSV1-UL48-582 | + | CCGUUGACGAACAUGAA | 17 | 6420 |
| HSV1-UL48-583 | + | UGACGAACAUGAAGGGC | 17 | 6421 |
| HSV1-UL48-584 | + | ACUGACGCCAGCUCUCC | 17 | 6422 |
| HSV1-UL48-585 | + | UCUCCAGGUCGCAACAG | 17 | 6423 |
| HSV1-UL48-586 | + | AACAGAGGCAGUCAAAC | 17 | 6424 |
| HSV1-UL48-587 | + | GAGGCAGUCAAACAGGU | 17 | 6425 |
| HSV1-UL48-588 | + | AGGCAGUCAAACAGGUC | 17 | 6426 |
| HSV1-UL48-589 | + | GGGCCGCAUCAUCUGCU | 17 | 6427 |
| HSV1-UL48-590 | + | CAUCUGCUCGGCGUACG | 17 | 6428 |
| HSV1-UL48-591 | + | CGGCGUACGCGGCCCAU | 17 | 6429 |
| HSV1-UL48-592 | + | CGGCCCAUAGGAUCUCG | 17 | 6430 |
| HSV1-UL48-593 | + | GGCCCAUAGGAUCUCGC | 17 | 6431 |
| HSV1-UL48-594 | + | AACACGCGCCAGACGAG | 17 | 6432 |
| HSV1-UL48-595 | + | CCAGACGAGCGGUCUCU | 17 | 6433 |
| HSV1-UL48-596 | + | GUACCUGUCCGCGAUCG | 17 | 6434 |
| HSV1-UL48-597 | + | CGCGCAGCAUUUCUCCC | 17 | 6435 |
| HSV1-UL48-598 | + | GUCCGCGCAUGUGCGCC | 17 | 6436 |
| HSV1-UL48-599 | + | CGCGCAUGUGCGCCUGG | 17 | 6437 |
| HSV1-UL48-600 | + | CCUGGCGGUGCAGCUGC | 17 | 6438 |
| HSV1-UL48-601 | + | GUGCAGCUGCCGGACGC | 17 | 6439 |
| HSV1-UL48-602 | + | GCCGGACGCUGGCGCGC | 17 | 6440 |
| HSV1-UL48-603 | + | CGCUGGCGCGCAGGUAC | 17 | 6441 |
| HSV1-UL48-604 | + | CGCGCAGGUACCGGUAC | 17 | 6442 |
| HSV1-UL48-605 | + | GCGCAGGUACCGGUACA | 17 | 6443 |
| HSV1-UL48-606 | + | CAGGGCCGAGCAGAAGU | 17 | 6444 |
| HSV1-UL48-607 | + | GCAGAAGUUGGCCAACA | 17 | 6445 |

Table 3D provides exemplary targeting domains for knocking out the UL48 gene selected according to the first tier parameters. The targeting domains are selected based on location within first 500 bp of the coding sequence of the UL48 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 3D

| 1st Tier | | | Target Site | Seq |
|---|---|---|---|---|
| gRNA Name | DNA Strand | Targeting Domain | Length | ID |
| HSV1-UL48-608 | − | UCGACGAGCUGUUUGCCGAC | 20 | 6446 |
| HSV1-UL48-609 | − | AGCUGUUUGCCGACAUGAAC | 20 | 6447 |
| HSV1-UL48-610 | − | GCCACCGCCCCCCCGCCCGG | 20 | 6448 |
| HSV1-UL48-4 | − | CCACCGCCCCCCCGCCCGGC | 20 | 5963 |
| HSV1-UL48-5 | − | CACCGCCCCCCCGCCCGGCC | 20 | 5964 |
| HSV1-UL48-613 | − | CGGCCCCCCCGCUGUACGCA | 20 | 6449 |
| HSV1-UL48-10 | − | GGCCCCCCCGCUGUACGCAA | 20 | 5840 |
| HSV1-UL48-615 | − | CGCUGUACGCAACGGGGCGC | 20 | 6450 |
| HSV1-UL48-616 | − | UUAACCGUCUCCUCGACGAC | 20 | 6451 |
| HSV1-UL48-617 | − | UCGACGACUUGGGCUUUAGC | 20 | 6452 |
| HSV1-UL48-618 | − | CUAUGUACCAUGCUCGAUAC | 20 | 6453 |
| HSV1-UL48-18 | − | UAUGUACCAUGCUCGAUACC | 20 | 5967 |
| HSV1-UL48-620 | − | UACCAUGCUCGAUACCUGGA | 20 | 6454 |
| HSV1-UL48-621 | − | CCAUGCUCGAUACCUGGAAC | 20 | 6455 |
| HSV1-UL48-622 | − | CCGACCAACGCCGACCUGUA | 20 | 6456 |
| HSV1-UL48-21 | − | CGACCAACGCCGACCUGUAC | 20 | 5860 |
| HSV1-UL48-22 | − | GACCAACGCCGACCUGUACC | 20 | 5861 |
| HSV1-UL48-625 | − | CAACGCUGCCCAGCGAUGUG | 20 | 6457 |
| HSV1-UL48-24 | − | AACGCUGCCCAGCGAUGUGG | 20 | 5863 |
| HSV1-UL48-627 | − | CUGCCCAGCGAUGUGGUGGA | 20 | 6458 |
| HSV1-UL48-25 | − | UGCCCAGCGAUGUGGUGGAA | 20 | 5864 |
| HSV1-UL48-26 | − | GCCCAGCGAUGUGGUGGAAU | 20 | 5968 |
| HSV1-UL48-27 | − | CCCAGCGAUGUGGUGGAAUG | 20 | 5969 |
| HSV1-UL48-28 | − | CCAGCGAUGUGGUGGAAUGG | 20 | 5970 |
| HSV1-UL48-632 | − | AUGGGGGGACGCGUACGUCC | 20 | 6459 |
| HSV1-UL48-633 | − | UCCGGCCACCCGCGACGGCC | 20 | 6460 |
| HSV1-UL48-634 | − | CGACGGCCUCGGGCUCUACU | 20 | 6461 |
| HSV1-UL48-635 | − | GCUCUCUCGUUUCUUCCACG | 20 | 6462 |
| HSV1-UL48-636 | − | CGUUUCUUCCACGCCGAGCU | 20 | 6463 |
| HSV1-UL48-637 | − | UUCCACGCCGAGCUACGGGC | 20 | 6464 |
| HSV1-UL48-38 | − | UCCACGCCGAGCUACGGGCG | 20 | 5879 |
| HSV1-UL48-39 | − | CCACGCCGAGCUACGGGCGC | 20 | 5880 |
| HSV1-UL48-640 | − | ACGCCGAGCUACGGGCGCGG | 20 | 6465 |
| HSV1-UL48-40 | − | CGCCGAGCUACGGGCGCGGG | 20 | 5973 |
| HSV1-UL48-642 | − | CCGAGCUACGGGCGCGGGAG | 20 | 6466 |
| HSV1-UL48-643 | − | ACGAGCUGUUUGCCGAC | 17 | 6467 |
| HSV1-UL48-644 | − | UGUUUGCCGACAUGAAC | 17 | 6468 |
| HSV1-UL48-645 | − | ACCGCCCCCCCGCCCGG | 17 | 6469 |
| HSV1-UL48-122 | − | CCGCCCCCCCGCCCGGC | 17 | 5977 |
| HSV1-UL48-123 | − | CGCCCCCCCGCCCGGCC | 17 | 5978 |
| HSV1-UL48-648 | − | CCCCCCCGCUGUACGCA | 17 | 6470 |
| HSV1-UL48-128 | − | CCCCCCGCUGUACGCAA | 17 | 5850 |
| HSV1-UL48-650 | − | UGUACGCAACGGGGCGC | 17 | 6471 |
| HSV1-UL48-651 | − | ACCGUCUCCUCGACGAC | 17 | 6472 |
| HSV1-UL48-652 | − | ACGACUUGGGCUUUAGC | 17 | 6473 |
| HSV1-UL48-653 | − | UGUACCAUGCUCGAUAC | 17 | 6474 |
| HSV1-UL48-136 | − | GUACCAUGCUCGAUACC | 17 | 5981 |
| HSV1-UL48-655 | − | CAUGCUCGAUACCUGGA | 17 | 6475 |
| HSV1-UL48-656 | − | UGCUCGAUACCUGGAAC | 17 | 6476 |
| HSV1-UL48-657 | − | ACCAACGCCGACCUGUA | 17 | 6477 |
| HSV1-UL48-139 | − | CCAACGCCGACCUGUAC | 17 | 5867 |
| HSV1-UL48-140 | − | CAACGCCGACCUGUACC | 17 | 5868 |
| HSV1-UL48-660 | − | CGCUGCCCAGCGAUGUG | 17 | 6478 |
| HSV1-UL48-142 | − | GCUGCCCAGCGAUGUGG | 17 | 5870 |
| HSV1-UL48-662 | − | CCCAGCGAUGUGGUGGA | 17 | 6479 |
| HSV1-UL48-143 | − | CCAGCGAUGUGGUGGAA | 17 | 5871 |
| HSV1-UL48-144 | − | CAGCGAUGUGGUGGAAU | 17 | 5982 |
| HSV1-UL48-145 | − | AGCGAUGUGGUGGAAUG | 17 | 5983 |
| HSV1-UL48-146 | − | GCGAUGUGGUGGAAUGG | 17 | 5984 |
| HSV1-UL48-667 | − | GGGGGACGCGUACGUCC | 17 | 6480 |
| HSV1-UL48-668 | − | GGCCACCCGCGACGGCC | 17 | 6481 |
| HSV1-UL48-669 | − | CGGCCUCGGGCUCUACU | 17 | 6482 |
| HSV1-UL48-670 | − | CUCUCGUUUCUUCCACG | 17 | 6483 |
| HSV1-UL48-671 | − | UUCUUCCACGCCGAGCU | 17 | 6484 |
| HSV1-UL48-672 | − | CACGCCGAGCUACGGGC | 17 | 6485 |
| HSV1-UL48-156 | − | ACGCCGAGCUACGGGCG | 17 | 5888 |
| HSV1-UL48-157 | − | CGCCGAGCUACGGGCGC | 17 | 5889 |
| HSV1-UL48-675 | − | CCGAGCUACGGGCGCGG | 17 | 6486 |
| HSV1-UL48-158 | − | CGAGCUACGGGCGCGGG | 17 | 5987 |
| HSV1-UL48-677 | − | AGCUACGGGCGCGGGAG | 17 | 6487 |
| HSV1-UL48-678 | + | CGGCAAACAGCUCGUCGACC | 20 | 6488 |
| HSV1-UL48-679 | + | GGCCGGGCGGGGGGGCGGUG | 20 | 6489 |
| HSV1-UL48-112 | + | UGGGACCCCCGGCCGGGCGG | 20 | 6025 |
| HSV1-UL48-111 | + | UUGGGACCCCCGGCCGGGCG | 20 | 6024 |
| HSV1-UL48-110 | + | UUUGGGACCCCCGGCCGGGC | 20 | 6023 |
| HSV1-UL48-109 | + | UUUUGGGACCCCCGGCCGGG | 20 | 6022 |
| HSV1-UL48-684 | + | UUUUUGGGACCCCCGGCCGG | 20 | 6490 |
| HSV1-UL48-685 | + | GGUGUUUUUGGGACCCCCGG | 20 | 6491 |
| HSV1-UL48-104 | + | GGGGGCCGCCGGGGUGUUUU | 20 | 5948 |
| HSV1-UL48-687 | + | GGGGGGCCGCCGGGGUGUUU | 20 | 6492 |
| HSV1-UL48-101 | + | GCGUACAGCGGGGGGGCCGC | 20 | 6020 |
| HSV1-UL48-689 | + | UGCGUACAGCGGGGGGGCCG | 20 | 6493 |
| HSV1-UL48-98 | + | CGCCCCGUUGCGUACAGCGG | 20 | 5939 |
| HSV1-UL48-97 | + | GCGCCCCGUUGCGUACAGCG | 20 | 5938 |
| HSV1-UL48-96 | + | GGCGCCCCGUUGCGUACAGC | 20 | 6019 |
| HSV1-UL48-95 | + | AGGCGCCCCGUUGCGUACAG | 20 | 6018 |
| HSV1-UL48-694 | + | CAGGCGCCCCGUUGCGUACA | 20 | 6494 |
| HSV1-UL48-695 | + | AUGGGUGGGGAGGGCAUGAG | 20 | 6495 |
| HSV1-UL48-696 | + | CGGGCAUGGGUGGGGAGGGC | 20 | 6496 |
| HSV1-UL48-697 | + | GGGGGACGGGCAUGGGUGGG | 20 | 6497 |
| HSV1-UL48-88 | + | GGGGGGGACGGGCAUGGGUG | 20 | 6011 |
| HSV1-UL48-87 | + | CGGGGGGGACGGGCAUGGGU | 20 | 6010 |
| HSV1-UL48-86 | + | GCGGGGGGGACGGGCAUGGG | 20 | 6009 |
| HSV1-UL48-701 | + | GGCGGGGGGGACGGGCAUGG | 20 | 6498 |
| HSV1-UL48-702 | + | GGGCGGCGGGGGGGACGGGC | 20 | 6499 |
| HSV1-UL48-703 | + | UAAAGAGGGCGGCGGGGGGG | 20 | 6500 |
| HSV1-UL48-80 | + | CGGUUAAAGAGGGCGGCGGG | 20 | 5932 |
| HSV1-UL48-79 | + | ACGGUUAAAGAGGGCGGCGG | 20 | 5931 |
| HSV1-UL48-78 | + | GACGGUUAAAGAGGGCGGCG | 20 | 5930 |
| HSV1-UL48-77 | + | AGACGGUUAAAGAGGGCGGC | 20 | 5929 |
| HSV1-UL48-76 | + | GAGACGGUUAAAGAGGGCGG | 20 | 5928 |
| HSV1-UL48-709 | + | GGAGACGGUUAAAGAGGGCG | 20 | 6501 |
| HSV1-UL48-710 | + | UCGUCGAGGAGACGGUUAAA | 20 | 6502 |
| HSV1-UL48-711 | + | AGUCGUCGAGGAGACGGUUA | 20 | 6503 |
| HSV1-UL48-71 | + | CGCUAAAGCCCAAGUCGUCG | 20 | 5915 |
| HSV1-UL48-713 | + | GCGCUAAAGCCCAAGUCGUC | 20 | 6504 |
| HSV1-UL48-714 | + | CCGCGCUAAAGCCCAAGUCG | 20 | 6505 |
| HSV1-UL48-68 | + | AUCGAGCAUGGUACAUAGCG | 20 | 5912 |
| HSV1-UL48-716 | + | UAUCGAGCAUGGUACAUAGC | 20 | 6506 |
| HSV1-UL48-717 | + | ACAGAUCCUCGUUCCAGGUA | 20 | 6507 |
| HSV1-UL48-718 | + | GUCGGCGUUGGUCGGUAGCG | 20 | 6508 |
| HSV1-UL48-60 | + | CAUCGCUGGGCAGCGUUGAU | 20 | 5998 |
| HSV1-UL48-720 | + | ACAUCGCUGGGCAGCGUUGA | 20 | 6509 |
| HSV1-UL48-721 | + | CCCCCCAUUCCACCACAUCG | 20 | 6510 |
| HSV1-UL48-56 | + | AUGUCGAUUUGGGUGCGUUC | 20 | 5995 |
| HSV1-UL48-55 | + | AAUGUCGAUUUGGGUGCGUU | 20 | 5994 |
| HSV1-UL48-724 | + | GAAUGUCGAUUUGGGUGCGU | 20 | 6511 |
| HSV1-UL48-725 | + | CCGUGGGCGCGAAUGUCGAU | 20 | 6512 |
| HSV1-UL48-726 | + | AAGGCCACGUCGCCGUGGGC | 20 | 6513 |
| HSV1-UL48-727 | + | GUAGGGAAGGCCACGUCGCC | 20 | 6514 |
| HSV1-UL48-49 | + | CGCGGGUGGCCGGAAGCGUA | 20 | 5992 |
| HSV1-UL48-48 | + | UCGCGGGUGGCCGGAAGCGU | 20 | 5896 |
| HSV1-UL48-730 | + | GUCGCGGGUGGCCGGAAGCG | 20 | 6515 |
| HSV1-UL48-47 | + | CCGAGGCCGUCGCGGGUGGC | 20 | 5991 |
| HSV1-UL48-732 | + | CCCGAGGCCGUCGCGGGUGG | 20 | 6516 |
| HSV1-UL48-733 | + | UAGUAGAGCCCGAGGCCGUC | 20 | 6517 |
| HSV1-UL48-734 | + | AGAGCGCUUCGUAGUAGAGC | 20 | 6518 |
| HSV1-UL48-735 | + | AACGAGAGAGCGCUUCGUAG | 20 | 6519 |
| HSV1-UL48-736 | + | GCUCGGCGUGGAAGAAACGA | 20 | 6520 |
| HSV1-UL48-737 | + | UAGCUCGGCGUGGAAGAAAC | 20 | 6521 |
| HSV1-UL48-738 | + | CGUAGCUCGGCGUGGAAGAA | 20 | 6522 |
| HSV1-UL48-739 | + | GCGCCCGUAGCUCGGCGUGG | 20 | 6523 |
| HSV1-UL48-42 | + | CCCGCGCCCGUAGCUCGGCG | 20 | 5988 |
| HSV1-UL48-741 | + | UCCCGCGCCCGUAGCUCGGC | 20 | 6524 |
| HSV1-UL48-742 | + | CAAACAGCUCGUCGACC | 17 | 6525 |
| HSV1-UL48-743 | + | CGGGCGGGGGGGCGGUG | 17 | 6526 |
| HSV1-UL48-230 | + | GACCCCCGGCCGGGCGG | 17 | 6068 |
| HSV1-UL48-229 | + | GGACCCCCGGCCGGGCG | 17 | 6067 |
| HSV1-UL48-228 | + | GGGACCCCCGGCCGGGC | 17 | 6066 |
| HSV1-UL48-227 | + | UGGGACCCCCGGCCGGG | 17 | 6065 |
| HSV1-UL48-748 | + | UUGGGACCCCCGGCCGG | 17 | 6527 |
| HSV1-UL48-749 | + | GUUUUUGGGACCCCCGG | 17 | 6528 |
| HSV1-UL48-222 | + | GGCCGCCGGGGUGUUUU | 17 | 5952 |
| HSV1-UL48-751 | + | GGGCCGCCGGGGUGUUU | 17 | 6529 |
| HSV1-UL48-219 | + | UACAGCGGGGGGGCCGC | 17 | 6063 |
| HSV1-UL48-753 | + | GUACAGCGGGGGGGCCG | 17 | 6530 |
| HSV1-UL48-216 | + | CCCGUUGCGUACAGCGG | 17 | 5943 |
| HSV1-UL48-215 | + | CCCCGUUGCGUACAGCG | 17 | 5942 |
| HSV1-UL48-214 | + | GCCCCGUUGCGUACAGC | 17 | 6062 |
| HSV1-UL48-213 | + | CGCCCCGUUGCGUACAG | 17 | 6061 |
| HSV1-UL48-758 | + | GCGCCCCGUUGCGUACA | 17 | 6531 |
| HSV1-UL48-759 | + | GGUGGGGAGGGCAUGAG | 17 | 6532 |
| HSV1-UL48-760 | + | GCAUGGGUGGGGAGGGC | 17 | 6533 |
| HSV1-UL48-761 | + | GGACGGGCAUGGGUGGG | 17 | 6534 |
| HSV1-UL48-206 | + | GGGGACGGGCAUGGGUG | 17 | 6054 |
| HSV1-UL48-205 | + | GGGGGACGGGCAUGGGU | 17 | 6053 |
| HSV1-UL48-204 | + | GGGGGGACGGGCAUGGG | 17 | 6052 |
| HSV1-UL48-765 | + | GGGGGGGACGGGCAUGG | 17 | 6535 |
| HSV1-UL48-766 | + | CGGCGGGGGGGACGGGC | 17 | 6536 |
| HSV1-UL48-767 | + | AGAGGGCGGCGGGGGGG | 17 | 6537 |
| HSV1-UL48-198 | + | UUAAAGAGGGCGGCGGG | 17 | 5937 |
| HSV1-UL48-197 | + | GUUAAAGAGGGCGGCGG | 17 | 5936 |
| HSV1-UL48-196 | + | GGUUAAAGAGGGCGGCG | 17 | 5935 |
| HSV1-UL48-195 | + | CGGUUAAAGAGGGCGGC | 17 | 5934 |
| HSV1-UL48-194 | + | ACGGUUAAAGAGGGCGG | 17 | 5933 |
| HSV1-UL48-773 | + | GACGGUUAAAGAGGGCG | 17 | 6538 |
| HSV1-UL48-774 | + | UCGAGGAGACGGUUAAA | 17 | 6539 |
| HSV1-UL48-775 | + | CGUCGAGGAGACGGUUA | 17 | 6540 |
| HSV1-UL48-189 | + | UAAAGCCCAAGUCGUCG | 17 | 5923 |
| HSV1-UL48-777 | + | CUAAAGCCCAAGUCGUC | 17 | 6541 |
| HSV1-UL48-778 | + | CGCUAAAGCCCAAGUCG | 17 | 6542 |
| HSV1-UL48-186 | + | GAGCAUGGUACAUAGCG | 17 | 5920 |
| HSV1-UL48-780 | + | CGAGCAUGGUACAUAGC | 17 | 6543 |
| HSV1-UL48-781 | + | GAUCCUCGUUCCAGGUA | 17 | 6544 |
| HSV1-UL48-782 | + | GGCGUUGGUCGGUAGCG | 17 | 6545 |
| HSV1-UL48-178 | + | CGCUGGGCAGCGUUGAU | 17 | 6041 |
| HSV1-UL48-784 | + | UCGCUGGGCAGCGUUGA | 17 | 6546 |
| HSV1-UL48-785 | + | CCCAUUCCACCACAUCG | 17 | 6547 |
| HSV1-UL48-174 | + | UCGAUUUGGGUGCGUUC | 17 | 6038 |
| HSV1-UL48-173 | + | GUCGAUUUGGGUGCGUU | 17 | 6037 |
| HSV1-UL48-788 | + | UGUCGAUUUGGGUGCGU | 17 | 6548 |
| HSV1-UL48-789 | + | UGGGCGCGAAUGUCGAU | 17 | 6549 |
| HSV1-UL48-790 | + | GCCACGUCGCCGUGGGC | 17 | 6550 |
| HSV1-UL48-791 | + | GGGAAGGCCACGUCGCC | 17 | 6551 |
| HSV1-UL48-167 | + | GGGUGGCCGGAAGCGUA | 17 | 6035 |
| HSV1-UL48-166 | + | CGGGUGGCCGGAAGCGU | 17 | 5897 |
| HSV1-UL48-794 | + | GCGGGUGGCCGGAAGCG | 17 | 6552 |
| HSV1-UL48-165 | + | AGGCCGUCGCGGGUGGC | 17 | 6034 |
| HSV1-UL48-796 | + | GAGGCCGUCGCGGGUGG | 17 | 6553 |
| HSV1-UL48-797 | + | UAGAGCCCGAGGCCGUC | 17 | 6554 |
| HSV1-UL48-798 | + | GCGCUUCGUAGUAGAGC | 17 | 6555 |
| HSV1-UL48-799 | + | GAGAGAGCGCUUCGUAG | 17 | 6556 |
| HSV1-UL48-800 | + | CGGCGUGGAAGAAACGA | 17 | 6557 |
| HSV1-UL48-801 | + | CUCGGCGUGGAAGAAAC | 17 | 6558 |
| HSV1-UL48-802 | + | AGCUCGGCGUGGAAGAA | 17 | 6559 |
| HSV1-UL48-803 | + | CCCGUAGCUCGGCGUGG | 17 | 6560 |
| HSV1-UL48-160 | + | GCGCCCGUAGCUCGGCG | 17 | 6031 |
| HSV1-UL48-805 | + | CGCGCCCGUAGCUCGGC | 17 | 6561 |

Table 3E provides exemplary targeting domains for knocking out the UL48 gene selected according to the second tier parameters. The targeting domains are selected based on location within the coding sequence, but downstream of the first 500 bp of the UL48 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 3E

| 2nd Tier | DNA | | Target Site | |
|---|---|---|---|---|
| gRNA Name | Strand | Targeting Domain | Length | Seq ID |
| HSV1-UL48-806 | − | GCACCGCCAGGCGCACAUGC | 20 | 6562 |
| HSV1-UL48-807 | − | UGCGCGGACGCGAUCGCGAC | 20 | 6563 |
| HSV1-UL48-241 | − | GCGCGGACGCGAUCGCGACC | 20 | 6079 |
| HSV1-UL48-242 | − | CGCGGACGCGAUCGCGACCU | 20 | 6080 |
| HSV1-UL48-810 | − | CGGACGCGAUCGCGACCUGG | 20 | 6564 |
| HSV1-UL48-811 | − | AAAUGCUGCGCGCCACGAUC | 20 | 6565 |
| HSV1-UL48-812 | − | ACGAUCGCGGACAGGUACUA | 20 | 6566 |
| HSV1-UL48-813 | − | GAUCGCGGACAGGUACUACC | 20 | 6567 |
| HSV1-UL48-814 | − | UUUGUAUCUAUUUUUGACCC | 20 | 6568 |
| HSV1-UL48-815 | − | UUUUUGACCCGCGAGAUCCU | 20 | 6569 |
| HSV1-UL48-816 | − | GAUCCUAUGGGCCGCGUACG | 20 | 6570 |
| HSV1-UL48-817 | − | UUGACUGCCUCUGUUGCGAC | 20 | 6571 |
| HSV1-UL48-249 | − | UGACUGCCUCUGUUGCGACC | 20 | 6087 |
| HSV1-UL48-819 | − | ACUGCCUCUGUUGCGACCUG | 20 | 6572 |
| HSV1-UL48-820 | − | UGGAGAGCUGGCGUCAGUUG | 20 | 6573 |
| HSV1-UL48-821 | − | CCAGCCCUUCAUGUUCGUCA | 20 | 6574 |
| HSV1-UL48-254 | − | CAGCCCUUCAUGUUCGUCAA | 20 | 6092 |
| HSV1-UL48-823 | − | GUCAACGGAGCGCUCACCGU | 20 | 6575 |
| HSV1-UL48-255 | − | UCAACGGAGCGCUCACCGUC | 20 | 6093 |
| HSV1-UL48-256 | − | CAACGGAGCGCUCACCGUCC | 20 | 6094 |
| HSV1-UL48-257 | − | AACGGAGCGCUCACCGUCCG | 20 | 6095 |
| HSV1-UL48-827 | − | CACCGUCCGGGGAGUGCCAA | 20 | 6576 |
| HSV1-UL48-828 | − | CCAAUCGAGGCCCGCCGGCU | 20 | 6577 |
| HSV1-UL48-260 | − | CAAUCGAGGCCCGCCGGCUG | 20 | 6098 |
| HSV1-UL48-261 | − | AAUCGAGGCCCGCCGGCUGC | 20 | 6099 |
| HSV1-UL48-831 | − | GCGGGAGCUAAACCACAUUC | 20 | 6578 |
| HSV1-UL48-832 | − | CGCUGGUGCGCAGCGCGGCU | 20 | 6579 |
| HSV1-UL48-264 | − | GCUGGUGCGCAGCGCGGCUA | 20 | 6102 |
| HSV1-UL48-834 | − | UGGUGCGCAGCGCGGCUACG | 20 | 6580 |
| HSV1-UL48-265 | − | GGUGCGCAGCGCGGCUACGG | 20 | 6103 |
| HSV1-UL48-836 | − | CAGCGCGGCUACGGAGGAGC | 20 | 6581 |
| HSV1-UL48-266 | − | AGCGCGGCUACGGAGGAGCC | 20 | 6104 |
| HSV1-UL48-838 | − | UGGCAACCAGGCCCGCGCCU | 20 | 6582 |
| HSV1-UL48-839 | − | GGGUACUUUAUGGUGUUGAU | 20 | 6583 |
| HSV1-UL48-840 | − | ACUUUAUGGUGUUGAUUCGG | 20 | 6584 |
| HSV1-UL48-841 | − | UGGUGUUGAUUCGGGCGAAG | 20 | 6585 |
| HSV1-UL48-842 | − | CAGCUUCACGACCUCGCCCU | 20 | 6586 |
| HSV1-UL48-843 | − | UCGCCCUCCGAGGCGGUCAU | 20 | 6587 |
| HSV1-UL48-279 | − | CGCCCUCCGAGGCGGUCAUG | 20 | 6117 |
| HSV1-UL48-280 | − | GCCCUCCGAGGCGGUCAUGC | 20 | 6118 |
| HSV1-UL48-846 | − | ACGCGUACAGCCGCGCGCGU | 20 | 6588 |
| HSV1-UL48-847 | − | CGCGCGUACGAAAAACAAUU | 20 | 6589 |
| HSV1-UL48-848 | − | AAACAAUUACGGGUCUACCA | 20 | 6590 |
| HSV1-UL48-849 | − | ACAAUUACGGGUCUACCAUC | 20 | 6591 |
| HSV1-UL48-850 | − | UCGAGGGCCUGCUCGAUCUC | 20 | 6592 |
| HSV1-UL48-851 | − | UCUCCCGGACGACGACGCCC | 20 | 6593 |
| HSV1-UL48-852 | − | CCCGGACGACGACGCCCCCG | 20 | 6594 |
| HSV1-UL48-853 | − | ACGACGACGCCCCCGAAGAG | 20 | 6595 |
| HSV1-UL48-287 | − | CGACGACGCCCCCGAAGAGG | 20 | 6125 |
| HSV1-UL48-855 | − | CGCGCCUGUCCUUUCUCCCC | 20 | 6596 |
| HSV1-UL48-292 | − | GCGCCUGUCCUUUCUCCCCG | 20 | 6130 |
| HSV1-UL48-857 | − | CCCCCCCGACCGAUGUCAGC | 20 | 6597 |
| HSV1-UL48-295 | − | CCCCCCGACCGAUGUCAGCC | 20 | 6133 |
| HSV1-UL48-296 | − | CCCCCGACCGAUGUCAGCCU | 20 | 6134 |
| HSV1-UL48-297 | − | CCCCGACCGAUGUCAGCCUG | 20 | 6135 |
| HSV1-UL48-861 | − | GACCGAUGUCAGCCUGGGGG | 20 | 6598 |
| HSV1-UL48-862 | − | GGACGAGCUCCACUUAGACG | 20 | 6599 |
| HSV1-UL48-863 | − | ACGAGCUCCACUUAGACGGC | 20 | 6600 |
| HSV1-UL48-864 | − | ACGCGCUAGACGAUUUCGAU | 20 | 6601 |
| HSV1-UL48-865 | − | ACGAUUUCGAUCUGGACAUG | 20 | 6602 |
| HSV1-UL48-304 | − | CGAUUUCGAUCUGGACAUGU | 20 | 6142 |
| HSV1-UL48-305 | − | GAUUUCGAUCUGGACAUGUU | 20 | 6143 |
| HSV1-UL48-306 | − | AUUUCGAUCUGGACAUGUUG | 20 | 6144 |
| HSV1-UL48-869 | − | CGAUCUGGACAUGUUGGGGG | 20 | 6603 |
| HSV1-UL48-308 | − | GAUCUGGACAUGUUGGGGGA | 20 | 6146 |
| HSV1-UL48-309 | − | AUCUGGACAUGUUGGGGGAC | 20 | 6147 |
| HSV1-UL48-872 | − | UGUUGGGGGACGGGGAUUCC | 20 | 6604 |
| HSV1-UL48-311 | − | GUUGGGGGACGGGGAUUCCC | 20 | 6149 |
| HSV1-UL48-874 | − | GGGACGGGGAUUCCCCGGGG | 20 | 6605 |
| HSV1-UL48-314 | − | GGACGGGGAUUCCCCGGGGC | 20 | 6152 |
| HSV1-UL48-876 | − | ACUCCGCCCCCUACGGCGCU | 20 | 6606 |
| HSV1-UL48-877 | − | CGCUCUGGAUAUGGCCGACU | 20 | 6607 |
| HSV1-UL48-878 | − | GGAUAUGGCCGACUUCGAGU | 20 | 6608 |
| HSV1-UL48-879 | − | GCAGAUGUUUACCGAUGCCC | 20 | 6609 |
| HSV1-UL48-319 | − | CAGAUGUUUACCGAUGCCCU | 20 | 6157 |
| HSV1-UL48-881 | − | UACCGAUGCCCUUGGAAUUG | 20 | 6610 |
| HSV1-UL48-882 | − | CCUUGGAAUUGACGAGUACG | 20 | 6611 |
| HSV1-UL48-883 | − | CCGCCAGGCGCACAUGC | 17 | 6612 |
| HSV1-UL48-884 | − | GCGGACGCGAUCGCGAC | 17 | 6613 |
| HSV1-UL48-427 | − | CGGACGCGAUCGCGACC | 17 | 6265 |
| HSV1-UL48-428 | − | GGACGCGAUCGCGACCU | 17 | 6266 |
| HSV1-UL48-887 | − | ACGCGAUCGCGACCUGG | 17 | 6614 |
| HSV1-UL48-888 | − | UGCUGCGCGCCACGAUC | 17 | 6615 |
| HSV1-UL48-889 | − | AUCGCGGACAGGUACUA | 17 | 6616 |
| HSV1-UL48-890 | − | CGCGGACAGGUACUACC | 17 | 6617 |
| HSV1-UL48-891 | − | GUAUCUAUUUUUGACCC | 17 | 6618 |
| HSV1-UL48-892 | − | UUGACCCGCGAGAUCCU | 17 | 6619 |
| HSV1-UL48-893 | − | CCUAUGGGCCGCGUACG | 17 | 6620 |
| HSV1-UL48-894 | − | ACUGCCUCUGUUGCGAC | 17 | 6621 |
| HSV1-UL48-435 | − | CUGCCUCUGUUGCGACC | 17 | 6273 |
| HSV1-UL48-896 | − | GCCUCUGUUGCGACCUG | 17 | 6622 |
| HSV1-UL48-897 | − | AGAGCUGGCGUCAGUUG | 17 | 6623 |
| HSV1-UL48-898 | − | GCCCUUCAUGUUCGUCA | 17 | 6624 |
| HSV1-UL48-440 | − | CCCUUCAUGUUCGUCAA | 17 | 6278 |
| HSV1-UL48-900 | − | AACGGAGCGCUCACCGU | 17 | 6625 |
| HSV1-UL48-441 | − | ACGGAGCGCUCACCGUC | 17 | 6279 |
| HSV1-UL48-442 | − | CGGAGCGCUCACCGUCC | 17 | 6280 |
| HSV1-UL48-443 | − | GGAGCGCUCACCGUCCG | 17 | 6281 |
| HSV1-UL48-904 | − | CGUCCGGGGAGUGCCAA | 17 | 6626 |
| HSV1-UL48-905 | − | AUCGAGGCCCGCCGGCU | 17 | 6627 |
| HSV1-UL48-446 | − | UCGAGGCCCGCCGGCUG | 17 | 6284 |
| HSV1-UL48-447 | − | CGAGGCCCGCCGGCUGC | 17 | 6285 |
| HSV1-UL48-908 | − | GGAGCUAAACCACAUUC | 17 | 6628 |
| HSV1-UL48-909 | − | UGGUGCGCAGCGCGGCU | 17 | 6629 |
| HSV1-UL48-450 | − | GGUGCGCAGCGCGGCUA | 17 | 6288 |
| HSV1-UL48-911 | − | UGCGCAGCGCGGCUACG | 17 | 6630 |
| HSV1-UL48-451 | − | GCGCAGCGCGGCUACGG | 17 | 6289 |
| HSV1-UL48-913 | − | CGCGGCUACGGAGGAGC | 17 | 6631 |
| HSV1-UL48-452 | − | GCGGCUACGGAGGAGCC | 17 | 6290 |
| HSV1-UL48-915 | − | CAACCAGGCCCGCGCCU | 17 | 6632 |
| HSV1-UL48-916 | − | UACUUUAUGGUGUUGAU | 17 | 6633 |
| HSV1-UL48-917 | − | UUAUGGUGUUGAUUCGG | 17 | 6634 |
| HSV1-UL48-918 | − | UGUUGAUUCGGGCGAAG | 17 | 6635 |
| HSV1-UL48-919 | − | CUUCACGACCUCGCCCU | 17 | 6636 |
| HSV1-UL48-920 | − | CCCUCCGAGGCGGUCAU | 17 | 6637 |
| HSV1-UL48-465 | − | CCUCCGAGGCGGUCAUG | 17 | 6303 |
| HSV1-UL48-466 | − | CUCCGAGGCGGUCAUGC | 17 | 6304 |
| HSV1-UL48-923 | − | CGUACAGCCGCGCGCGU | 17 | 6638 |
| HSV1-UL48-924 | − | GCGUACGAAAAACAAUU | 17 | 6639 |
| HSV1-UL48-925 | − | CAAUUACGGGUCUACCA | 17 | 6640 |
| HSV1-UL48-926 | − | AUUACGGGUCUACCAUC | 17 | 6641 |
| HSV1-UL48-927 | − | AGGGCCUGCUCGAUCUC | 17 | 6642 |
| HSV1-UL48-928 | − | CCCGGACGACGACGCCC | 17 | 6643 |
| HSV1-UL48-929 | − | GGACGACGACGCCCCCG | 17 | 6644 |
| HSV1-UL48-930 | − | ACGACGCCCCCGAAGAG | 17 | 6645 |
| HSV1-UL48-473 | − | CGACGCCCCCGAAGAGG | 17 | 6311 |
| HSV1-UL48-932 | − | GCCUGUCCUUUCUCCCC | 17 | 6646 |
| HSV1-UL48-478 | − | CCUGUCCUUUCUCCCCG | 17 | 6316 |
| HSV1-UL48-934 | − | CCCCGACCGAUGUCAGC | 17 | 6647 |
| HSV1-UL48-481 | − | CCCGACCGAUGUCAGCC | 17 | 6319 |
| HSV1-UL48-482 | − | CCGACCGAUGUCAGCCU | 17 | 6320 |
| HSV1-UL48-483 | − | CGACCGAUGUCAGCCUG | 17 | 6321 |
| HSV1-UL48-938 | − | CGAUGUCAGCCUGGGGG | 17 | 6648 |
| HSV1-UL48-939 | − | CGAGCUCCACUUAGACG | 17 | 6649 |
| HSV1-UL48-940 | − | AGCUCCACUUAGACGGC | 17 | 6650 |
| HSV1-UL48-941 | − | CGCUAGACGAUUUCGAU | 17 | 6651 |
| HSV1-UL48-942 | − | AUUUCGAUCUGGACAUG | 17 | 6652 |
| HSV1-UL48-490 | − | UUUCGAUCUGGACAUGU | 17 | 6328 |
| HSV1-UL48-491 | − | UUCGAUCUGGACAUGUU | 17 | 6329 |
| HSV1-UL48-492 | − | UCGAUCUGGACAUGUUG | 17 | 6330 |
| HSV1-UL48-946 | − | UCUGGACAUGUUGGGGG | 17 | 6653 |
| HSV1-UL48-494 | − | CUGGACAUGUUGGGGGA | 17 | 6332 |
| HSV1-UL48-495 | − | UGGACAUGUUGGGGGAC | 17 | 6333 |
| HSV1-UL48-949 | − | UGGGGGACGGGGAUUCC | 17 | 6654 |
| HSV1-UL48-497 | − | GGGGGACGGGGAUUCCC | 17 | 6335 |
| HSV1-UL48-951 | − | ACGGGGAUUCCCCGGGG | 17 | 6655 |
| HSV1-UL48-500 | − | CGGGGAUUCCCCGGGGC | 17 | 6338 |
| HSV1-UL48-953 | − | CCGCCCCCUACGGCGCU | 17 | 6656 |
| HSV1-UL48-954 | − | UCUGGAUAUGGCCGACU | 17 | 6657 |
| HSV1-UL48-955 | − | UAUGGCCGACUUCGAGU | 17 | 6658 |
| HSV1-UL48-956 | − | GAUGUUUACCGAUGCCC | 17 | 6659 |
| HSV1-UL48-505 | − | AUGUUUACCGAUGCCCU | 17 | 6343 |
| HSV1-UL48-958 | − | CGAUGCCCUUGGAAUUG | 17 | 6660 |
| HSV1-UL48-959 | − | UGGAAUUGACGAGUACG | 17 | 6661 |
| HSV1-UL48-960 | + | GGUACCGGUACAGGGCCGAG | 20 | 6662 |
| HSV1-UL48-961 | + | GCGCAGGUACCGGUACAGGG | 20 | 6663 |
| HSV1-UL48-962 | + | CUGGCGCGCAGGUACCGGUA | 20 | 6664 |
| HSV1-UL48-963 | + | UGCGCCUGGCGGUGCAGCUG | 20 | 6665 |
| HSV1-UL48-964 | + | AAAAACAAAACACGCGCCAG | 20 | 6666 |
| HSV1-UL48-965 | + | UACGCGGCCCAUAGGAUCUC | 20 | 6667 |
| HSV1-UL48-966 | + | UGCUCGGCGUACGCGGCCCA | 20 | 6668 |
| HSV1-UL48-967 | + | AACAGAGGCAGUCAAACAGG | 20 | 6669 |
| HSV1-UL48-968 | + | GCCAGCUCUCCAGGUCGCAA | 20 | 6670 |
| HSV1-UL48-397 | + | CGUUGACGAACAUGAAGGGC | 20 | 6235 |
| HSV1-UL48-970 | + | CCGUUGACGAACAUGAAGGG | 20 | 6671 |
| HSV1-UL48-971 | + | GCGCUCCGUUGACGAACAUG | 20 | 6672 |
| HSV1-UL48-972 | + | UGAGCGCUCCGUUGACGAAC | 20 | 6673 |
| HSV1-UL48-973 | + | GGACGGUGAGCGCUCCGUUG | 20 | 6674 |
| HSV1-UL48-974 | + | CGAUUGGCACUCCCCGGACG | 20 | 6675 |
| HSV1-UL48-975 | + | CGGGCCUCGAUUGGCACUCC | 20 | 6676 |
| HSV1-UL48-976 | + | UGGUUUAGCUCCCGCAGCCG | 20 | 6677 |
| HSV1-UL48-977 | + | AGCGGGAGGUUAAGGUGCUC | 20 | 6678 |
| HSV1-UL48-385 | + | UAGCCGCGCUGCGCACCAGC | 20 | 6223 |
| HSV1-UL48-384 | + | GUAGCCGCGCUGCGCACCAG | 20 | 6222 |
| HSV1-UL48-980 | + | CGUAGCCGCGCUGCGCACCA | 20 | 6679 |
| HSV1-UL48-379 | + | UGGUUGCCAUGCAGGGUGGG | 20 | 6217 |
| HSV1-UL48-982 | + | CUGGUUGCCAUGCAGGGUGG | 20 | 6680 |
| HSV1-UL48-378 | + | GCCUGGUUGCCAUGCAGGGU | 20 | 6216 |
| HSV1-UL48-377 | + | GGCCUGGUUGCCAUGCAGGG | 20 | 6215 |
| HSV1-UL48-985 | + | GGGCCUGGUUGCCAUGCAGG | 20 | 6681 |
| HSV1-UL48-986 | + | GCGCGGGCCUGGUUGCCAUG | 20 | 6682 |
| HSV1-UL48-987 | + | ACCAUAAAGUACCCAGAGGC | 20 | 6683 |
| HSV1-UL48-988 | + | AAUCAACACCAUAAAGUACC | 20 | 6684 |
| HSV1-UL48-989 | + | GAAUACGAGUCCAACUUCGC | 20 | 6685 |
| HSV1-UL48-990 | + | CGAGGUCGUGAAGCUGGAAU | 20 | 6686 |
| HSV1-UL48-370 | + | GGAGGGCGAGGUCGUGAAGC | 20 | 6208 |
| HSV1-UL48-992 | + | CGGAGGGCGAGGUCGUGAAG | 20 | 6687 |
| HSV1-UL48-993 | + | CCGCCUCGGAGGGCGAGGUC | 20 | 6688 |
| HSV1-UL48-994 | + | CCGCAUGACCGCCUCGGAGG | 20 | 6689 |
| HSV1-UL48-995 | + | GUUCCCGCAUGACCGCCUCG | 20 | 6690 |
| HSV1-UL48-366 | + | GUGUUCCCGCAUGACCGCCU | 20 | 6204 |
| HSV1-UL48-997 | + | CGUGUUCCCGCAUGACCGCC | 20 | 6691 |
| HSV1-UL48-998 | + | GGGCGUCGUCGUCCGGGAGA | 20 | 6692 |
| HSV1-UL48-362 | + | CUUCGGGGGCGUCGUCGUCC | 20 | 6200 |
| HSV1-UL48-361 | + | UCUUCGGGGGCGUCGUCGUC | 20 | 6199 |
| HSV1-UL48-1001 | + | CUCUUCGGGGGCGUCGUCGU | 20 | 6693 |
| HSV1-UL48-358 | + | GCCGCCAGCCCCGCCUCUUC | 20 | 6196 |
| HSV1-UL48-357 | + | AGCCGCCAGCCCCGCCUCUU | 20 | 6195 |
| HSV1-UL48-1004 | + | GAGCCGCCAGCCCCGCCUCU | 20 | 6694 |
| HSV1-UL48-356 | + | GCGGGGAGAAAGGACAGGCG | 20 | 6194 |
| HSV1-UL48-1006 | + | CGCGGGGAGAAAGGACAGGC | 20 | 6695 |
| HSV1-UL48-1007 | + | GCGUGUGUCCCGCGGGGAGA | 20 | 6696 |
| HSV1-UL48-1008 | + | CUGCGCGUGUGUCCCGCGGG | 20 | 6697 |
| HSV1-UL48-353 | + | GUCUGCGCGUGUGUCCCGCG | 20 | 6191 |
| HSV1-UL48-352 | + | AGUCUGCGCGUGUGUCCCGC | 20 | 6190 |
| HSV1-UL48-351 | + | CAGUCUGCGCGUGUGUCCCG | 20 | 6189 |
| HSV1-UL48-1012 | + | ACAGUCUGCGCGUGUGUCCC | 20 | 6698 |
| HSV1-UL48-348 | + | CCCAGGCUGACAUCGGUCGG | 20 | 6186 |
| HSV1-UL48-347 | + | CCCCAGGCUGACAUCGGUCG | 20 | 6185 |
| HSV1-UL48-346 | + | CCCCCAGGCUGACAUCGGUC | 20 | 6184 |
| HSV1-UL48-345 | + | UCCCCCAGGCUGACAUCGGU | 20 | 6183 |
| HSV1-UL48-1017 | + | GUCCCCCAGGCUGACAUCGG | 20 | 6699 |
| HSV1-UL48-342 | + | CCACGUCCUCGCCGUCUAAG | 20 | 6180 |
| HSV1-UL48-1019 | + | GCCACGUCCUCGCCGUCUAA | 20 | 6700 |
| HSV1-UL48-1020 | + | CGUCCCCCAACAUGUCCAGA | 20 | 6701 |
| HSV1-UL48-340 | + | GGGGGUAAAUCCCGGCCCCG | 20 | 6178 |
| HSV1-UL48-339 | + | GGGGGGUAAAUCCCGGCCCC | 20 | 6177 |
| HSV1-UL48-338 | + | UGGGGGGUAAAUCCCGGCCC | 20 | 6176 |
| HSV1-UL48-1024 | + | GUGGGGGGUAAAUCCCGGCC | 20 | 6702 |
| HSV1-UL48-334 | + | CCGUAGGGGGCGGAGUCGUG | 20 | 6172 |
| HSV1-UL48-333 | + | GCCGUAGGGGGCGGAGUCGU | 20 | 6171 |
| HSV1-UL48-332 | + | CGCCGUAGGGGGCGGAGUCG | 20 | 6170 |
| HSV1-UL48-1028 | + | GCGCCGUAGGGGGCGGAGUC | 20 | 6703 |
| HSV1-UL48-331 | + | AUCCAGAGCGCCGUAGGGGG | 20 | 6169 |
| HSV1-UL48-1030 | + | UAUCCAGAGCGCCGUAGGGG | 20 | 6704 |
| HSV1-UL48-328 | + | GCCAUAUCCAGAGCGCCGUA | 20 | 6166 |
| HSV1-UL48-327 | + | GGCCAUAUCCAGAGCGCCGU | 20 | 6165 |
| HSV1-UL48-1033 | + | CGGCCAUAUCCAGAGCGCCG | 20 | 6705 |
| HSV1-UL48-1034 | + | AACUCGAAGUCGGCCAUAUC | 20 | 6706 |
| HSV1-UL48-1035 | + | CGGUAAACAUCUGCUCAAAC | 20 | 6707 |
| HSV1-UL48-1036 | + | CCACCGUACUCGUCAAUUCC | 20 | 6708 |
| HSV1-UL48-1037 | + | ACCGGUACAGGGCCGAG | 17 | 6709 |
| HSV1-UL48-1038 | + | CAGGUACCGGUACAGGG | 17 | 6710 |
| HSV1-UL48-1039 | + | GCGCGCAGGUACCGGUA | 17 | 6711 |
| HSV1-UL48-1040 | + | GCCUGGCGGUGCAGCUG | 17 | 6712 |
| HSV1-UL48-1041 | + | AACAAAACACGCGCCAG | 17 | 6713 |
| HSV1-UL48-1042 | + | GCGGCCCAUAGGAUCUC | 17 | 6714 |
| HSV1-UL48-1043 | + | UCGGCGUACGCGGCCCA | 17 | 6715 |
| HSV1-UL48-1044 | + | AGAGGCAGUCAAACAGG | 17 | 6716 |
| HSV1-UL48-1045 | + | AGCUCUCCAGGUCGCAA | 17 | 6717 |
| HSV1-UL48-583 | + | UGACGAACAUGAAGGGC | 17 | 6421 |
| HSV1-UL48-1047 | + | UUGACGAACAUGAAGGG | 17 | 6718 |
| HSV1-UL48-1048 | + | CUCCGUUGACGAACAUG | 17 | 6719 |
| HSV1-UL48-1049 | + | GCGCUCCGUUGACGAAC | 17 | 6720 |
| HSV1-UL48-1050 | + | CGGUGAGCGCUCCGUUG | 17 | 6721 |
| HSV1-UL48-1051 | + | UUGGCACUCCCCGGACG | 17 | 6722 |
| HSV1-UL48-1052 | + | GCCUCGAUUGGCACUCC | 17 | 6723 |
| HSV1-UL48-1053 | + | UUUAGCUCCCGCAGCCG | 17 | 6724 |
| HSV1-UL48-1054 | + | GGGAGGUUAAGGUGCUC | 17 | 6725 |
| HSV1-UL48-571 | + | CCGCGCUGCGCACCAGC | 17 | 6409 |
| HSV1-UL48-570 | + | GCCGCGCUGCGCACCAG | 17 | 6408 |
| HSV1-UL48-1057 | + | AGCCGCGCUGCGCACCA | 17 | 6726 |
| HSV1-UL48-565 | + | UUGCCAUGCAGGGUGGG | 17 | 6403 |
| HSV1-UL48-1059 | + | GUUGCCAUGCAGGGUGG | 17 | 6727 |
| HSV1-UL48-564 | + | UGGUUGCCAUGCAGGGU | 17 | 6402 |
| HSV1-UL48-563 | + | CUGGUUGCCAUGCAGGG | 17 | 6401 |
| HSV1-UL48-1062 | + | CCUGGUUGCCAUGCAGG | 17 | 6728 |
| HSV1-UL48-1063 | + | CGGGCCUGGUUGCCAUG | 17 | 6729 |
| HSV1-UL48-1064 | + | AUAAAGUACCCAGAGGC | 17 | 6730 |
| HSV1-UL48-1065 | + | CAACACCAUAAAGUACC | 17 | 6731 |
| HSV1-UL48-1066 | + | UACGAGUCCAACUUCGC | 17 | 6732 |
| HSV1-UL48-1067 | + | GGUCGUGAAGCUGGAAU | 17 | 6733 |
| HSV1-UL48-556 | + | GGGCGAGGUCGUGAAGC | 17 | 6394 |
| HSV1-UL48-1069 | + | AGGGCGAGGUCGUGAAG | 17 | 6734 |
| HSV1-UL48-1070 | + | CCUCGGAGGGCGAGGUC | 17 | 6735 |
| HSV1-UL48-1071 | + | CAUGACCGCCUCGGAGG | 17 | 6736 |
| HSV1-UL48-1072 | + | CCCGCAUGACCGCCUCG | 17 | 6737 |
| HSV1-UL48-552 | + | UUCCCGCAUGACCGCCU | 17 | 6390 |
| HSV1-UL48-1074 | + | GUUCCCGCAUGACCGCC | 17 | 6738 |
| HSV1-UL48-1075 | + | CGUCGUCGUCCGGGAGA | 17 | 6739 |
| HSV1-UL48-548 | + | CGGGGGCGUCGUCGUCC | 17 | 6386 |
| HSV1-UL48-547 | + | UCGGGGGCGUCGUCGUC | 17 | 6385 |
| HSV1-UL48-1078 | + | UUCGGGGGCGUCGUCGU | 17 | 6740 |
| HSV1-UL48-544 | + | GCCAGCCCCGCCUCUUC | 17 | 6382 |
| HSV1-UL48-543 | + | CGCCAGCCCCGCCUCUU | 17 | 6381 |
| HSV1-UL48-1081 | + | CCGCCAGCCCCGCCUCU | 17 | 6741 |
| HSV1-UL48-542 | + | GGGAGAAAGGACAGGCG | 17 | 6380 |
| HSV1-UL48-1083 | + | GGGGAGAAAGGACAGGC | 17 | 6742 |
| HSV1-UL48-1084 | + | UGUGUCCCGCGGGGAGA | 17 | 6743 |
| HSV1-UL48-1085 | + | CGCGUGUGUCCCGCGGG | 17 | 6744 |
| HSV1-UL48-539 | + | UGCGCGUGUGUCCCGCG | 17 | 6377 |
| HSV1-UL48-538 | + | CUGCGCGUGUGUCCCGC | 17 | 6376 |
| HSV1-UL48-537 | + | UCUGCGCGUGUGUCCCG | 17 | 6375 |
| HSV1-UL48-1089 | + | GUCUGCGCGUGUGUCCC | 17 | 6745 |
| HSV1-UL48-534 | + | AGGCUGACAUCGGUCGG | 17 | 6372 |
| HSV1-UL48-533 | + | CAGGCUGACAUCGGUCG | 17 | 6371 |
| HSV1-UL48-532 | + | CCAGGCUGACAUCGGUC | 17 | 6370 |
| HSV1-UL48-531 | + | CCCAGGCUGACAUCGGU | 17 | 6369 |
| HSV1-UL48-1094 | + | CCCCAGGCUGACAUCGG | 17 | 6746 |
| HSV1-UL48-528 | + | CGUCCUCGCCGUCUAAG | 17 | 6366 |
| HSV1-UL48-1096 | + | ACGUCCUCGCCGUCUAA | 17 | 6747 |
| HSV1-UL48-1097 | + | CCCCCAACAUGUCCAGA | 17 | 6748 |
| HSV1-UL48-526 | + | GGUAAAUCCCGGCCCCG | 17 | 6364 |
| HSV1-UL48-525 | + | GGGUAAAUCCCGGCCCC | 17 | 6363 |
| HSV1-UL48-524 | + | GGGGUAAAUCCCGGCCC | 17 | 6362 |
| HSV1-UL48-1101 | + | GGGGGUAAAUCCCGGCC | 17 | 6749 |
| HSV1-UL48-520 | + | UAGGGGGCGGAGUCGUG | 17 | 6358 |
| HSV1-UL48-519 | + | GUAGGGGGCGGAGUCGU | 17 | 6357 |
| HSV1-UL48-518 | + | CGUAGGGGGCGGAGUCG | 17 | 6356 |
| HSV1-UL48-1105 | + | CCGUAGGGGGCGGAGUC | 17 | 6750 |
| HSV1-UL48-517 | + | CAGAGCGCCGUAGGGGG | 17 | 6355 |
| HSV1-UL48-1107 | + | CCAGAGCGCCGUAGGGG | 17 | 6751 |
| HSV1-UL48-514 | + | AUAUCCAGAGCGCCGUA | 17 | 6352 |
| HSV1-UL48-513 | + | CAUAUCCAGAGCGCCGU | 17 | 6351 |
| HSV1-UL48-1110 | + | CCAUAUCCAGAGCGCCG | 17 | 6752 |
| HSV1-UL48-1111 | + | UCGAAGUCGGCCAUAUC | 17 | 6753 |
| HSV1-UL48-1112 | + | UAAACAUCUGCUCAAAC | 17 | 6754 |
| HSV1-UL48-1113 | + | CCGUACUCGUCAAUUCC | 17 | 6755 |

Table 3F provides exemplary targeting domains for knocking out the UL48 gene selected according to the first tier parameters. The targeting domains are selected based on location within first 500 bp of the coding sequence of the UL48 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 3F

| | | | Target | |
|---|---|---|---|---|
| 1st Tier | DNA | | Site | Seq |
| gRNA Name | Strand | Targeting Domain | Length | ID |
| HSV1-UL48- | + | CACGUCGCCGUGGGCGCGAA | 20 | 6756 |
| 1114 | | | | |
| HSV1-UL48- | + | GUCGCCGUGGGCGCGAA | 20 | 6757 |
| 1115 | | | | |

Table 3G provides exemplary targeting domains for knocking out the UL48 gene selected according to the second tier parameters. The targeting domains are selected based on location within the coding sequence (but downstream of the first 500 bp) of the UL48 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 3G

| 2nd Tier | DNA | | Target Site | |
|---|---|---|---|---|
| gRNA Name | Strand | Targeting Domain | Length | Seq ID |
| HSV1-UL48-1116 | − | CGCCUCUGGGUACUUUAUGG | 20 | 6758 |
| HSV1-UL48-1117 | − | AUGGCGCAUGCCGACGCGCU | 20 | 6759 |
| HSV1-UL48-308 | − | GAUCUGGACAUGUUGGGGGA | 20 | 6146 |
| HSV1-UL48-874 | − | GGGACGGGGAUUCCCCGGGG | 20 | 6605 |
| HSV1-UL48-1120 | − | CUCUGGGUACUUUAUGG | 17 | 6760 |
| HSV1-UL48-1121 | − | GCGCAUGCCGACGCGCU | 17 | 6761 |
| HSV1-UL48-494 | − | CUGGACAUGUUGGGGGA | 17 | 6332 |
| HSV1-UL48-951 | − | ACGGGGAUUCCCCGGGG | 17 | 6655 |
| HSV1-UL48-1124 | + | UAGCUCCCGCAGCCGGCGGG | 20 | 6762 |
| HSV1-UL48-1125 | + | CUCCCGCAGCCGGCGGG | 20 | 6763 |

Table 4A provides exemplary targeting domains for knocking out the UL54 gene selected according to first tier parameters. The targeting domains are selected based on location within the first 500 bp of the coding sequence of the UL54 gene and orthogonality against the human genome. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 4A

| 1st Tier | | | Target | |
|---|---|---|---|---|
| gRNA Name | DNA Strand | Targeting Domain | Site Length | Seq ID |
| HSV1-UL54-1 | − | GGACCUCUCCGACAGCGAUC | 20 | 6764 |
| HSV1-UL54-2 | − | CUCCGACAGCGAUCUGGACG | 20 | 6765 |
| HSV1-UL54-3 | − | CCGCCGCGACGACCUGGAAU | 20 | 6766 |
| HSV1-UL54-4 | − | CAGCGGGGAGUGUUCCUCGU | 20 | 6767 |
| HSV1-UL54-5 | − | UUCCUCGUCGGACGAGGACA | 20 | 6768 |
| HSV1-UL54-6 | − | GAUACUCGACGCCGCUCGCC | 20 | 6769 |
| HSV1-UL54-7 | − | ACUCGACGCCGCUCGCCCGG | 20 | 6770 |
| HSV1-UL54-8 | − | CACCCAGACGCCUCGUCCGA | 20 | 6771 |
| HSV1-UL54-9 | − | GCCUCGUCCGACGGAGCGGC | 20 | 6772 |
| HSV1-UL54-10 | − | ACAGUGUGUGGUCGCGCCUC | 20 | 6773 |
| HSV1-UL54-11 | − | CAGUGUGUGGUCGCGCCUCG | 20 | 6774 |
| HSV1-UL54-12 | − | UGUGGUCGCGCCUCGGGGCC | 20 | 6775 |
| HSV1-UL54-13 | − | UUGCUCCCCCGAGCAGCACG | 20 | 6776 |
| HSV1-UL54-14 | − | UCGGGGUCGCGGUGGUCCCG | 20 | 6777 |
| HSV1-UL54-15 | − | CGGGGCUGCCGAUGGUUUGU | 20 | 6778 |
| HSV1-UL54-16 | − | AUGGUUUGUCGGACCCCCGC | 20 | 6779 |
| HSV1-UL54-17 | + | GCGGGGGUCCGACAAACCAU | 20 | 6780 |
| HSV1-UL54-18 | + | CGACAAACCAUCGGCAGCCC | 20 | 6781 |
| HSV1-UL54-19 | + | GACGCCCACGGCGUCCGCCG | 20 | 6782 |
| HSV1-UL54-20 | + | CCUUGCCCCCGUGCUGCUCG | 20 | 6783 |
| HSV1-UL54-21 | + | CUUGCCCCCGUGCUGCUCGG | 20 | 6784 |
| HSV1-UL54-22 | + | CGGGGGAGCAAGACGGUCGC | 20 | 6785 |
| HSV1-UL54-23 | + | GGGGGAGCAAGACGGUCGCC | 20 | 6786 |
| HSV1-UL54-24 | + | CGAGGCGCGACCACACACUG | 20 | 6787 |
| HSV1-UL54-25 | + | GAGGCGCGACCACACACUGU | 20 | 6788 |
| HSV1-UL54-26 | + | UUGGGGCCCUGCCGCUCCGU | 20 | 6789 |
| HSV1-UL54-27 | + | CCCUGCCGCUCCGUCGGACG | 20 | 6790 |
| HSV1-UL54-28 | + | GCUCCGUCGGACGAGGCGUC | 20 | 6791 |
| HSV1-UL54-29 | + | CUCCGUCGGACGAGGCGUCU | 20 | 6792 |
| HSV1-UL54-30 | + | GGUCUUCUGGACGAGACGGG | 20 | 6793 |
| HSV1-UL54-31 | + | GGGCGAGCGGCGUCGAGUAU | 20 | 6794 |
| HSV1-UL54-32 | + | UCCGGUCCGUCCUCUCCGUG | 20 | 6795 |
| HSV1-UL54-33 | + | UUCCAUGUCCUCGUCCGACG | 20 | 6796 |
| HSV1-UL54-34 | + | UGUCCGAUUCCAGGUCGUCG | 20 | 6797 |
| HSV1-UL54-35 | + | CCGAUUCCAGGUCGUCGCGG | 20 | 6798 |
| HSV1-UL54-36 | + | CGGCUCUCCGCCGGCUCGGG | 20 | 6799 |
| HSV1-UL54-37 | + | GUCCUCGUCCAGAUCGCUGU | 20 | 6800 |
| HSV1-UL54-38 | + | CGUCCAGAUCGCUGUCGGAG | 20 | 6801 |
| HSV1-UL54-39 | + | GAUCGCUGUCGGAGAGGUCC | 20 | 6802 |
| HSV1-UL54-40 | + | UGUCGGAGAGGUCCAGGCCG | 20 | 6803 |
| HSV1-UL54-41 | − | CCUCUCCGACAGCGAUC | 17 | 6804 |
| HSV1-UL54-42 | − | CGACAGCGAUCUGGACG | 17 | 6805 |
| HSV1-UL54-43 | − | CCGCGACGACCUGGAAU | 17 | 6806 |
| HSV1-UL54-44 | − | CGGGGAGUGUUCCUCGU | 17 | 6807 |
| HSV1-UL54-45 | − | CUCGUCGGACGAGGACA | 17 | 6808 |
| HSV1-UL54-46 | − | ACUCGACGCCGCUCGCC | 17 | 6809 |
| HSV1-UL54-47 | − | CGACGCCGCUCGCCCGG | 17 | 6810 |
| HSV1-UL54-48 | − | CCAGACGCCUCGUCCGA | 17 | 6811 |
| HSV1-UL54-49 | − | UCGUCCGACGGAGCGGC | 17 | 6812 |
| HSV1-UL54-50 | − | GUGUGUGGUCGCGCCUC | 17 | 6813 |
| HSV1-UL54-51 | − | UGUGUGGUCGCGCCUCG | 17 | 6814 |
| HSV1-UL54-52 | − | GGUCGCGCCUCGGGGCC | 17 | 6815 |
| HSV1-UL54-53 | − | CUCCCCCGAGCAGCACG | 17 | 6816 |
| HSV1-UL54-54 | − | GGGUCGCGGUGGUCCCG | 17 | 6817 |
| HSV1-UL54-55 | − | GGCUGCCGAUGGUUUGU | 17 | 6818 |
| HSV1-UL54-56 | − | GUUUGUCGGACCCCCGC | 17 | 6819 |
| HSV1-UL54-57 | + | GGGGUCCGACAAACCAU | 17 | 6820 |
| HSV1-UL54-58 | + | CAAACCAUCGGCAGCCC | 17 | 6821 |
| HSV1-UL54-59 | + | GCCCACGGCGUCCGCCG | 17 | 6822 |
| HSV1-UL54-60 | + | UGCCCCCGUGCUGCUCG | 17 | 6823 |
| HSV1-UL54-61 | + | GCCCCCGUGCUGCUCGG | 17 | 6824 |
| HSV1-UL54-62 | + | GGGAGCAAGACGGUCGC | 17 | 6825 |
| HSV1-UL54-63 | + | GGAGCAAGACGGUCGCC | 17 | 6826 |
| HSV1-UL54-64 | + | GGCGCGACCACACACUG | 17 | 6827 |
| HSV1-UL54-65 | + | GCGCGACCACACACUGU | 17 | 6828 |
| HSV1-UL54-66 | + | GGGCCCUGCCGCUCCGU | 17 | 6829 |
| HSV1-UL54-67 | + | UGCCGCUCCGUCGGACG | 17 | 6830 |
| HSV1-UL54-68 | + | CCGUCGGACGAGGCGUC | 17 | 6831 |
| HSV1-UL54-69 | + | CGUCGGACGAGGCGUCU | 17 | 6832 |
| HSV1-UL54-70 | + | CUUCUGGACGAGACGGG | 17 | 6833 |
| HSV1-UL54-71 | + | CGAGCGGCGUCGAGUAU | 17 | 6834 |
| HSV1-UL54-72 | + | GGUCCGUCCUCUCCGUG | 17 | 6835 |
| HSV1-UL54-73 | + | CAUGUCCUCGUCCGACG | 17 | 6836 |
| HSV1-UL54-74 | + | CCGAUUCCAGGUCGUCG | 17 | 6837 |
| HSV1-UL54-75 | + | AUUCCAGGUCGUCGCGG | 17 | 6838 |
| HSV1-UL54-76 | + | CUCUCCGCCGGCUCGGG | 17 | 6839 |
| HSV1-UL54-77 | + | CUCGUCCAGAUCGCUGU | 17 | 6840 |
| HSV1-UL54-78 | + | CCAGAUCGCUGUCGGAG | 17 | 6841 |
| HSV1-UL54-79 | + | CGCUGUCGGAGAGGUCC | 17 | 6842 |
| HSV1-UL54-80 | + | CGGAGAGGUCCAGGCCG | 17 | 6843 |

Table 4B provides exemplary targeting domains for knocking out the UL54 gene selected according to the second tier parameters. The targeting domains are selected based on location within the first 500 bp of the coding sequence of the UL54 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 4B

| 2nd Tier | | | Target Site | |
|---|---|---|---|---|
| gRNA Name | DNA Strand | Targeting Domain | Length | Seq ID |
| HSV1-UL54-81 | − | AUUGAUAUGCUAAUUGACCU | 20 | 6844 |
| HSV1-UL54-82 | − | UAUGCUAAUUGACCUCGGCC | 20 | 6845 |
| HSV1-UL54-83 | − | GGACGAGGACCCCCCCGAGC | 20 | 6846 |
| HSV1-UL54-84 | − | CGAGGACCCCCCCGAGCCGG | 20 | 6847 |
| HSV1-UL54-85 | − | GGAGAGCCGCCGCGACGACC | 20 | 6848 |
| HSV1-UL54-86 | − | GACCUGGAAUCGGACAGCAG | 20 | 6849 |
| HSV1-UL54-87 | − | ACCUGGAAUCGGACAGCAGC | 20 | 6850 |
| HSV1-UL54-88 | − | CCUGGAAUCGGACAGCAGCG | 20 | 6851 |
| HSV1-UL54-89 | − | GGAGUGUUCCUCGUCGGACG | 20 | 6852 |
| HSV1-UL54-90 | − | GAGGACAUGGAAGACCCCCA | 20 | 6853 |
| HSV1-UL54-91 | − | CAUGGAAGACCCCCACGGAG | 20 | 6854 |
| HSV1-UL54-92 | − | GAAGACCCCCACGGAGAGGA | 20 | 6855 |
| HSV1-UL54-93 | − | CCCCCACGGAGAGGACGGAC | 20 | 6856 |
| HSV1-UL54-94 | − | CCGUCUCGUCCAGAAGACCC | 20 | 6857 |
| HSV1-UL54-95 | − | AGACGCCUCGUCCGACGGAG | 20 | 6858 |
| HSV1-UL54-96 | − | CCUCGUCCGACGGAGCGGCA | 20 | 6859 |
| HSV1-UL54-97 | − | AACCAGCGCCCCACAGUGUG | 20 | 6860 |
| HSV1-UL54-98 | − | CACAGUGUGUGGUCGCGCCU | 20 | 6861 |
| HSV1-UL54-99 | − | UCUUGCUCCCCCGAGCAGCA | 20 | 6862 |
| HSV1-UL54-100 | − | CUUGCUCCCCCGAGCAGCAC | 20 | 6863 |
| HSV1-UL54-101 | − | UGCUCCCCCGAGCAGCACGG | 20 | 6864 |
| HSV1-UL54-102 | − | CCCCGAGCAGCACGGGGGCA | 20 | 6865 |
| HSV1-UL54-103 | − | CGAGCAGCACGGGGGCAAGG | 20 | 6866 |
| HSV1-UL54-104 | − | ACCAAAGCCCAGCCUGCCCG | 20 | 6867 |
| HSV1-UL54-105 | − | AAAGCCCAGCCUGCCCGCGG | 20 | 6868 |
| HSV1-UL54-106 | − | CCUGCCCGCGGCGGACGCCG | 20 | 6869 |
| HSV1-UL54-107 | − | CUGCCCGCGGCGGACGCCGU | 20 | 6870 |
| HSV1-UL54-108 | − | GCGGACGCCGUGGGCGUCGC | 20 | 6871 |
| HSV1-UL54-109 | − | CGGACGCCGUGGGCGUCGCA | 20 | 6872 |
| HSV1-UL54-110 | − | GGACGCCGUGGGCGUCGCAG | 20 | 6873 |
| HSV1-UL54-111 | − | GCCGUGGGCGUCGCAGGGGU | 20 | 6874 |
| HSV1-UL54-112 | − | CCGUGGGCGUCGCAGGGGUC | 20 | 6875 |
| HSV1-UL54-113 | − | CGUGGGCGUCGCAGGGGUCG | 20 | 6876 |
| HSV1-UL54-114 | − | CGUCGCAGGGGUCGGGGUCG | 20 | 6877 |
| HSV1-UL54-115 | − | CGCAGGGGUCGGGGUCGCGG | 20 | 6878 |
| HSV1-UL54-116 | − | GGUCGGGGUCGCGGUGGUCC | 20 | 6879 |
| HSV1-UL54-117 | − | GUCGGGGUCGCGGUGGUCCC | 20 | 6880 |
| HSV1-UL54-118 | − | GGUGGUCCCGGGGCUGCCGA | 20 | 6881 |
| HSV1-UL54-119 | + | GACAAACCAUCGGCAGCCCC | 20 | 6882 |
| HSV1-UL54-120 | + | CCCGACCCCUGCGACGCCCA | 20 | 6883 |
| HSV1-UL54-121 | + | ACGCCCACGGCGUCCGCCGC | 20 | 6884 |
| HSV1-UL54-122 | + | CCACGGCGUCCGCCGCGGGC | 20 | 6885 |
| HSV1-UL54-123 | + | GGCGUCCGCCGCGGGCAGGC | 20 | 6886 |
| HSV1-UL54-124 | + | GCGUCCGCCGCGGGCAGGCU | 20 | 6887 |
| HSV1-UL54-125 | + | GCCGCGGGCAGGCUGGGCUU | 20 | 6888 |
| HSV1-UL54-126 | + | CGGGCAGGCUGGGCUUUGGU | 20 | 6889 |
| HSV1-UL54-127 | + | GCAGGCUGGGCUUUGGUCGG | 20 | 6890 |
| HSV1-UL54-128 | + | CAGGCUGGGCUUUGGUCGGU | 20 | 6891 |
| HSV1-UL54-129 | + | AGGCUGGGCUUUGGUCGGUG | 20 | 6892 |
| HSV1-UL54-130 | + | GGCUGGGCUUUGGUCGGUGG | 20 | 6893 |
| HSV1-UL54-131 | + | GGGCUUUGGUCGGUGGGGGU | 20 | 6894 |
| HSV1-UL54-132 | + | CUUUGGUCGGUGGGGGUUGG | 20 | 6895 |
| HSV1-UL54-133 | + | UGGUCGGUGGGGGUUGGAGG | 20 | 6896 |
| HSV1-UL54-134 | + | GGUCGGUGGGGGUUGGAGGC | 20 | 6897 |
| HSV1-UL54-135 | + | CACCUUGCCCCCGUGCUGCU | 20 | 6898 |
| HSV1-UL54-136 | + | ACCUUGCCCCCGUGCUGCUC | 20 | 6899 |
| HSV1-UL54-137 | + | UGCUGCUCGGGGGAGCAAGA | 20 | 6900 |
| HSV1-UL54-138 | + | AAGACGGUCGCCGGGCCCCG | 20 | 6901 |
| HSV1-UL54-139 | + | AGGCGCGACCACACACUGUG | 20 | 6902 |
| HSV1-UL54-140 | + | GACCACACACUGUGGGGCGC | 20 | 6903 |
| HSV1-UL54-141 | + | ACACUGUGGGGCGCUGGUUG | 20 | 6904 |
| HSV1-UL54-142 | + | GGGCGCUGGUUGAGGAUCGU | 20 | 6905 |
| HSV1-UL54-143 | + | GGCGCUGGUUGAGGAUCGUU | 20 | 6906 |
| HSV1-UL54-144 | + | GCGCUGGUUGAGGAUCGUUG | 20 | 6907 |
| HSV1-UL54-145 | + | CGGACGAGGCGUCUGGGUGC | 20 | 6908 |
| HSV1-UL54-146 | + | GGACGAGGCGUCUGGGUGCU | 20 | 6909 |
| HSV1-UL54-147 | + | CGUCUGGGUGCUGGGUACGC | 20 | 6910 |
| HSV1-UL54-148 | + | GUCUGGGUGCUGGGUACGCC | 20 | 6911 |
| HSV1-UL54-149 | + | UCUGGGUGCUGGGUACGCCG | 20 | 6912 |
| HSV1-UL54-150 | + | CUGGGUACGCCGGGGUCUUC | 20 | 6913 |
| HSV1-UL54-151 | + | CCGGGGUCUUCUGGACGAGA | 20 | 6914 |
| HSV1-UL54-152 | + | CGGGGUCUUCUGGACGAGAC | 20 | 6915 |
| HSV1-UL54-153 | + | GGACGAGACGGGCGGACCGC | 20 | 6916 |
| HSV1-UL54-154 | + | GACGAGACGGGCGGACCGCC | 20 | 6917 |
| HSV1-UL54-155 | + | CGGGCGGACCGCCGGGCGAG | 20 | 6918 |
| HSV1-UL54-156 | + | GCGGCGUCGAGUAUCGGCUC | 20 | 6919 |
| HSV1-UL54-157 | + | GCUCCGGUCCGUCCUCUCCG | 20 | 6920 |
| HSV1-UL54-158 | + | CUCCGGUCCGUCCUCUCCGU | 20 | 6921 |
| HSV1-UL54-159 | + | CCGGUCCGUCCUCUCCGUGG | 20 | 6922 |
| HSV1-UL54-160 | + | CCCCGCUGCUGUCCGAUUCC | 20 | 6923 |
| HSV1-UL54-161 | + | UCGUCGCGGCGGCUCUCCGC | 20 | 6924 |
| HSV1-UL54-162 | + | GCGGCGGCUCUCCGCCGGCU | 20 | 6925 |
| HSV1-UL54-163 | + | CGGCGGCUCUCCGCCGGCUC | 20 | 6926 |
| HSV1-UL54-164 | + | GGCGGCUCUCCGCCGGCUCG | 20 | 6927 |
| HSV1-UL54-165 | + | GCGGCUCUCCGCCGGCUCGG | 20 | 6928 |
| HSV1-UL54-166 | + | GGCUCUCCGCCGGCUCGGGG | 20 | 6929 |
| HSV1-UL54-167 | − | GAUAUGCUAAUUGACCU | 17 | 6930 |
| HSV1-UL54-168 | − | GCUAAUUGACCUCGGCC | 17 | 6931 |
| HSV1-UL54-169 | − | CGAGGACCCCCCCGAGC | 17 | 6932 |
| HSV1-UL54-170 | − | GGACCCCCCCGAGCCGG | 17 | 6933 |
| HSV1-UL54-171 | − | GAGCCGCCGCGACGACC | 17 | 6934 |
| HSV1-UL54-172 | − | CUGGAAUCGGACAGCAG | 17 | 6935 |
| HSV1-UL54-173 | − | UGGAAUCGGACAGCAGC | 17 | 6936 |
| HSV1-UL54-174 | − | GGAAUCGGACAGCAGCG | 17 | 6937 |
| HSV1-UL54-175 | − | GUGUUCCUCGUCGGACG | 17 | 6938 |
| HSV1-UL54-176 | − | GACAUGGAAGACCCCCA | 17 | 6939 |
| HSV1-UL54-177 | − | GGAAGACCCCCACGGAG | 17 | 6940 |
| HSV1-UL54-178 | − | GACCCCCACGGAGAGGA | 17 | 6941 |
| HSV1-UL54-179 | − | CCACGGAGAGGACGGAC | 17 | 6942 |
| HSV1-UL54-180 | − | UCUCGUCCAGAAGACCC | 17 | 6943 |
| HSV1-UL54-181 | − | CGCCUCGUCCGACGGAG | 17 | 6944 |
| HSV1-UL54-182 | − | CGUCCGACGGAGCGGCA | 17 | 6945 |
| HSV1-UL54-183 | − | CAGCGCCCCACAGUGUG | 17 | 6946 |
| HSV1-UL54-184 | − | AGUGUGUGGUCGCGCCU | 17 | 6947 |
| HSV1-UL54-185 | − | UGCUCCCCCGAGCAGCA | 17 | 6948 |
| HSV1-UL54-186 | − | GCUCCCCCGAGCAGCAC | 17 | 6949 |
| HSV1-UL54-187 | − | UCCCCCGAGCAGCACGG | 17 | 6950 |
| HSV1-UL54-188 | − | CGAGCAGCACGGGGGCA | 17 | 6951 |
| HSV1-UL54-189 | − | GCAGCACGGGGGCAAGG | 17 | 6952 |
| HSV1-UL54-190 | − | AAAGCCCAGCCUGCCCG | 17 | 6953 |
| HSV1-UL54-191 | − | GCCCAGCCUGCCCGCGG | 17 | 6954 |
| HSV1-UL54-192 | − | GCCCGCGGCGGACGCCG | 17 | 6955 |
| HSV1-UL54-193 | − | CCCGCGGCGGACGCCGU | 17 | 6956 |
| HSV1-UL54-194 | − | GACGCCGUGGGCGUCGC | 17 | 6957 |
| HSV1-UL54-195 | − | ACGCCGUGGGCGUCGCA | 17 | 6958 |
| HSV1-UL54-196 | − | CGCCGUGGGCGUCGCAG | 17 | 6959 |
| HSV1-UL54-197 | − | GUGGGCGUCGCAGGGGU | 17 | 6960 |
| HSV1-UL54-198 | − | UGGGCGUCGCAGGGGUC | 17 | 6961 |
| HSV1-UL54-199 | − | GGGCGUCGCAGGGGUCG | 17 | 6962 |
| HSV1-UL54-200 | − | CGCAGGGGUCGGGGUCG | 17 | 6963 |
| HSV1-UL54-201 | − | AGGGGUCGGGGUCGCGG | 17 | 6964 |
| HSV1-UL54-202 | − | CGGGGUCGCGGUGGUCC | 17 | 6965 |
| HSV1-UL54-203 | − | GGGGUCGCGGUGGUCCC | 17 | 6966 |
| HSV1-UL54-204 | − | GGUCCCGGGGCUGCCGA | 17 | 6967 |
| HSV1-UL54-205 | + | AAACCAUCGGCAGCCCC | 17 | 6968 |
| HSV1-UL54-206 | + | GACCCCUGCGACGCCCA | 17 | 6969 |
| HSV1-UL54-207 | + | CCCACGGCGUCCGCCGC | 17 | 6970 |
| HSV1-UL54-208 | + | CGGCGUCCGCCGCGGGC | 17 | 6971 |
| HSV1-UL54-209 | + | GUCCGCCGCGGGCAGGC | 17 | 6972 |
| HSV1-UL54-210 | + | UCCGCCGCGGGCAGGCU | 17 | 6973 |
| HSV1-UL54-211 | + | GCGGGCAGGCUGGGCUU | 17 | 6974 |
| HSV1-UL54-212 | + | GCAGGCUGGGCUUUGGU | 17 | 6975 |
| HSV1-UL54-213 | + | GGCUGGGCUUUGGUCGG | 17 | 6976 |
| HSV1-UL54-214 | + | GCUGGGCUUUGGUCGGU | 17 | 6977 |
| HSV1-UL54-215 | + | CUGGGCUUUGGUCGGUG | 17 | 6978 |
| HSV1-UL54-216 | + | UGGGCUUUGGUCGGUGG | 17 | 6979 |
| HSV1-UL54-217 | + | CUUUGGUCGGUGGGGGU | 17 | 6980 |
| HSV1-UL54-218 | + | UGGUCGGUGGGGGUUGG | 17 | 6981 |
| HSV1-UL54-219 | + | UCGGUGGGGGUUGGAGG | 17 | 6982 |
| HSV1-UL54-220 | + | CGGUGGGGGUUGGAGGC | 17 | 6983 |
| HSV1-UL54-221 | + | CUUGCCCCCGUGCUGCU | 17 | 6984 |
| HSV1-UL54-222 | + | UUGCCCCCGUGCUGCUC | 17 | 6985 |
| HSV1-UL54-223 | + | UGCUCGGGGGAGCAAGA | 17 | 6986 |
| HSV1-UL54-224 | + | ACGGUCGCCGGGCCCCG | 17 | 6987 |
| HSV1-UL54-225 | + | CGCGACCACACACUGUG | 17 | 6988 |
| HSV1-UL54-226 | + | CACACACUGUGGGGCGC | 17 | 6989 |
| HSV1-UL54-227 | + | CUGUGGGGCGCUGGUUG | 17 | 6990 |
| HSV1-UL54-228 | + | CGCUGGUUGAGGAUCGU | 17 | 6991 |
| HSV1-UL54-229 | + | GCUGGUUGAGGAUCGUU | 17 | 6992 |
| HSV1-UL54-230 | + | CUGGUUGAGGAUCGUUG | 17 | 6993 |
| HSV1-UL54-231 | + | ACGAGGCGUCUGGGUGC | 17 | 6994 |
| HSV1-UL54-232 | + | CGAGGCGUCUGGGUGCU | 17 | 6995 |
| HSV1-UL54-233 | + | CUGGGUGCUGGGUACGC | 17 | 6996 |
| HSV1-UL54-234 | + | UGGGUGCUGGGUACGCC | 17 | 6997 |
| HSV1-UL54-235 | + | GGGUGCUGGGUACGCCG | 17 | 6998 |
| HSV1-UL54-236 | + | GGUACGCCGGGGUCUUC | 17 | 6999 |
| HSV1-UL54-237 | + | GGGUCUUCUGGACGAGA | 17 | 7000 |
| HSV1-UL54-238 | + | GGUCUUCUGGACGAGAC | 17 | 7001 |
| HSV1-UL54-239 | + | CGAGACGGGCGGACCGC | 17 | 7002 |
| HSV1-UL54-240 | + | GAGACGGGCGGACCGCC | 17 | 7003 |
| HSV1-UL54-241 | + | GCGGACCGCCGGGCGAG | 17 | 7004 |
| HSV1-UL54-242 | + | GCGUCGAGUAUCGGCUC | 17 | 7005 |
| HSV1-UL54-243 | + | CCGGUCCGUCCUCUCCG | 17 | 7006 |
| HSV1-UL54-244 | + | CGGUCCGUCCUCUCCGU | 17 | 7007 |
| HSV1-UL54-245 | + | GUCCGUCCUCUCCGUGG | 17 | 7008 |
| HSV1-UL54-246 | + | CGCUGCUGUCCGAUUCC | 17 | 7009 |
| HSV1-UL54-247 | + | UCGCGGCGGCUCUCCGC | 17 | 7010 |
| HSV1-UL54-248 | + | GCGGCUCUCCGCCGGCU | 17 | 7011 |
| HSV1-UL54-249 | + | CGGCUCUCCGCCGGCUC | 17 | 7012 |
| HSV1-UL54-250 | + | GGCUCUCCGCCGGCUCG | 17 | 7013 |
| HSV1-UL54-251 | + | GCUCUCCGCCGGCUCGG | 17 | 7014 |
| HSV1-UL54-252 | + | UCUCCGCCGGCUCGGGG | 17 | 7015 |

Table 4C provides exemplary targeting domains for knocking out the UL54 gene selected according to the third tier parameters. The targeting domains are selected based on location within the coding sequence, but downstream of the first 500 bp of the UL54 gene. It is contemplated herein that the targeting domain hybridizes to the target domain through complementary base pairing. Any of the targeting domains in the table can be used with a S. pyogenes Cas9 molecule that gives double stranded cleavage. Any of the targeting domains in the table can be used with a S. pyogenes Cas9 single-stranded break nucleases (nickases). In an embodiment, dual targeting is used to create two nicks on opposite DNA strands by using S. pyogenes Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 4C

| 3rd Tier | | | Target Site | |
|---|---|---|---|---|
| gRNA Name | DNA Strand | Targeting Domain | Length | Seq ID |
| HSV1-UL54-253 | − | AGAACCAAUCGCAACCCUGG | 20 | 7016 |
| HSV1-UL54-254 | − | AACCCUGGGGGACCCCGCCC | 20 | 7017 |
| HSV1-UL54-255 | − | ACCCUGGGGGACCCCGCCCC | 20 | 7018 |
| HSV1-UL54-256 | − | CCCUGGGGGACCCCGCCCCG | 20 | 7019 |
| HSV1-UL54-257 | − | UGGGGGACCCCGCCCCGGGG | 20 | 7020 |
| HSV1-UL54-258 | − | GGGGGACCCCGCCCCGGGGC | 20 | 7021 |
| HSV1-UL54-259 | − | GGGGACCCCGCCCCGGGGCG | 20 | 7022 |
| HSV1-UL54-260 | − | GACCCCGCCCCGGGGCGGGG | 20 | 7023 |
| HSV1-UL54-261 | − | CCGCCCCGGGGCGGGGUGGA | 20 | 7024 |
| HSV1-UL54-262 | − | CCCGGGGCGGGGUGGACGGA | 20 | 7025 |
| HSV1-UL54-263 | − | GCGGGGUGGACGGACGGCCC | 20 | 7026 |
| HSV1-UL54-264 | − | GACGGCCCCGGCGCCCCCCA | 20 | 7027 |
| HSV1-UL54-265 | − | CCCCGGCGCCCCCCAUGGCG | 20 | 7028 |
| HSV1-UL54-266 | − | GCGCCCCCCAUGGCGAGGCG | 20 | 7029 |
| HSV1-UL54-267 | − | CCCCAUGGCGAGGCGUGGCG | 20 | 7030 |
| HSV1-UL54-268 | − | AGUGAGCAGCCCGACCCACC | 20 | 7031 |
| HSV1-UL54-269 | − | GAGCAGCCCGACCCACCCGG | 20 | 7032 |
| HSV1-UL54-270 | − | CCGACCCACCCGGAGGCCAG | 20 | 7033 |
| HSV1-UL54-271 | − | CACCCGGAGGCCAGCGGACA | 20 | 7034 |
| HSV1-UL54-272 | − | ACCCGGAGGCCAGCGGACAC | 20 | 7035 |
| HSV1-UL54-273 | − | CCCGGAGGCCAGCGGACACG | 20 | 7036 |
| HSV1-UL54-274 | − | ACCCCCCCCGCUAAUGACGC | 20 | 7037 |
| HSV1-UL54-275 | − | GGCGAUUGCCCCCCCGCCCG | 20 | 7038 |
| HSV1-UL54-276 | − | GCCCGCGGACCCCCGCGCCC | 20 | 7039 |
| HSV1-UL54-277 | − | GGACCCCCGCGCCCCGGCCC | 20 | 7040 |
| HSV1-UL54-278 | − | CGCCCCGGCCCCGGAGCGAA | 20 | 7041 |
| HSV1-UL54-279 | − | ACACCAUCGACGCCACCACG | 20 | 7042 |
| HSV1-UL54-280 | − | CAUCGACGCCACCACGCGGU | 20 | 7043 |
| HSV1-UL54-281 | − | GCGCUCCAUCUCCGAGCGCG | 20 | 7044 |
| HSV1-UL54-282 | − | CUCCAUCUCCGAGCGCGCGG | 20 | 7045 |
| HSV1-UL54-283 | − | GACCGCAUCAGCGAGAGCUU | 20 | 7046 |
| HSV1-UL54-284 | − | GAGCUUUGGCCGCAGCGCAC | 20 | 7047 |
| HSV1-UL54-285 | − | CAGGUCAUGCACGACCCCUU | 20 | 7048 |
| HSV1-UL54-286 | − | AGGUCAUGCACGACCCCUUU | 20 | 7049 |
| HSV1-UL54-287 | − | GGUCAUGCACGACCCCUUUG | 20 | 7050 |
| HSV1-UL54-288 | − | GUCAUGCACGACCCCUUUGG | 20 | 7051 |
| HSV1-UL54-289 | − | UCAUGCACGACCCCUUUGGG | 20 | 7052 |
| HSV1-UL54-290 | − | UUCCCGCCGCGAAUAGCCCC | 20 | 7053 |
| HSV1-UL54-291 | − | UCCCGCCGCGAAUAGCCCCU | 20 | 7054 |
| HSV1-UL54-292 | − | CGCGAAUAGCCCCUGGGCCC | 20 | 7055 |
| HSV1-UL54-293 | − | UAGCCCCUGGGCCCCGGUGC | 20 | 7056 |
| HSV1-UL54-294 | − | CCCCUGGGCCCCGGUGCUGG | 20 | 7057 |
| HSV1-UL54-295 | − | CCCUGGGCCCCGGUGCUGGC | 20 | 7058 |
| HSV1-UL54-296 | − | GCCCCGGUGCUGGCGGGCCA | 20 | 7059 |
| HSV1-UL54-297 | − | CCGGUGCUGGCGGGCCAAGG | 20 | 7060 |
| HSV1-UL54-298 | − | CGGUGCUGGCGGGCCAAGGA | 20 | 7061 |
| HSV1-UL54-299 | − | CCUUUGACGCCGAGACCAGA | 20 | 7062 |
| HSV1-UL54-300 | − | CUUUGACGCCGAGACCAGAC | 20 | 7063 |
| HSV1-UL54-301 | − | CCGAGACCAGACGGGUCUCC | 20 | 7064 |
| HSV1-UL54-302 | − | CGAGACCAGACGGGUCUCCU | 20 | 7065 |
| HSV1-UL54-303 | − | ACGGGUCUCCUGGGAAACCU | 20 | 7066 |
| HSV1-UL54-304 | − | UGGGAAACCUUGGUCGCCCA | 20 | 7067 |
| HSV1-UL54-305 | − | AGCCUCUAUCGCACUUUUGC | 20 | 7068 |
| HSV1-UL54-306 | − | GCACUUUUGCCGGCAAUCCU | 20 | 7069 |
| HSV1-UL54-307 | − | CACUUUUGCCGGCAAUCCUC | 20 | 7070 |
| HSV1-UL54-308 | − | UCGGGCCGCAUCGACCGCCA | 20 | 7071 |
| HSV1-UL54-309 | − | GCGCCAAGAAAAUUUCAUCG | 20 | 7072 |
| HSV1-UL54-310 | − | AGAAAAUUUCAUCGAGGCGC | 20 | 7073 |
| HSV1-UL54-311 | − | GGCCUCCGCCGACGAGACGC | 20 | 7074 |
| HSV1-UL54-312 | − | CCGCCGACGAGACGCUGGCG | 20 | 7075 |
| HSV1-UL54-313 | − | CAACCUGCCGCUGCGCCCCC | 20 | 7076 |
| HSV1-UL54-314 | − | CGCCCCCAGGACCCCAUUAU | 20 | 7077 |
| HSV1-UL54-315 | − | GCCCCCAGGACCCCAUUAUC | 20 | 7078 |
| HSV1-UL54-316 | − | CCCCAUUAUCGGGACGACCG | 20 | 7079 |
| HSV1-UL54-317 | − | CGGGACGACCGCGGCUGUGC | 20 | 7080 |
| HSV1-UL54-318 | − | AUAACCUCGCCACGCGCCUG | 20 | 7081 |
| HSV1-UL54-319 | − | CUUUCUCCAGUGCUACCUGA | 20 | 7082 |
| HSV1-UL54-320 | − | CAGUGCUACCUGAAGGCGCG | 20 | 7083 |
| HSV1-UL54-321 | − | CUGAAGGCGCGAGGCCUGUG | 20 | 7084 |
| HSV1-UL54-322 | − | GGCGCGAGGCCUGUGCGGCC | 20 | 7085 |
| HSV1-UL54-323 | − | GCCUGGACGAACUGUGUUCG | 20 | 7086 |
| HSV1-UL54-324 | − | UGGACGAACUGUGUUCGCGG | 20 | 7087 |
| HSV1-UL54-325 | − | ACUGUGUUCGCGGCGGCGUC | 20 | 7088 |
| HSV1-UL54-326 | − | GUGUUCGCGGCGGCGUCUGG | 20 | 7089 |
| HSV1-UL54-327 | − | GCGGCGUCUGGCGGACAUUA | 20 | 7090 |
| HSV1-UL54-328 | − | AUCCUUCGUGUUUGUCAUUC | 20 | 7091 |
| HSV1-UL54-329 | − | UCGUGUUUGUCAUUCUGGCC | 20 | 7092 |
| HSV1-UL54-330 | − | CUCGCCAACCGCGUCGAGCG | 20 | 7093 |
| HSV1-UL54-331 | − | CCGCGUCGAGCGUGGCGUCG | 20 | 7094 |
| HSV1-UL54-332 | − | GAGAUCGACUACGCGACCCU | 20 | 7095 |
| HSV1-UL54-333 | − | GACUACGCGACCCUUGGUGU | 20 | 7096 |
| HSV1-UL54-334 | − | ACUACGCGACCCUUGGUGUC | 20 | 7097 |
| HSV1-UL54-335 | − | CUACGCGACCCUUGGUGUCG | 20 | 7098 |
| HSV1-UL54-336 | − | GCGACCCUUGGUGUCGGGGU | 20 | 7099 |
| HSV1-UL54-337 | − | AAGAUGCAUUUCUACCUCCC | 20 | 7100 |
| HSV1-UL54-338 | − | AGAUGCAUUUCUACCUCCCC | 20 | 7101 |
| HSV1-UL54-339 | − | GAUGCAUUUCUACCUCCCCG | 20 | 7102 |
| HSV1-UL54-340 | − | CUACCUCCCCGGGGCCUGCA | 20 | 7103 |
| HSV1-UL54-341 | − | CCUCCCCGGGGCCUGCAUGG | 20 | 7104 |
| HSV1-UL54-342 | − | CUCCCCGGGGCCUGCAUGGC | 20 | 7105 |
| HSV1-UL54-343 | − | AAUCCUAGACACGCACCGCC | 20 | 7106 |
| HSV1-UL54-344 | − | GAGUCGUGUCUGCGAGUUGA | 20 | 7107 |
| HSV1-UL54-345 | − | GUCGCCCCCCCGUACGUGCA | 20 | 7108 |
| HSV1-UL54-346 | + | AAAUAUUUGCCGUGCACGUA | 20 | 7109 |
| HSV1-UL54-347 | + | AAUAUUUGCCGUGCACGUAC | 20 | 7110 |
| HSV1-UL54-348 | + | AUAUUUGCCGUGCACGUACG | 20 | 7111 |
| HSV1-UL54-349 | + | UAUUUGCCGUGCACGUACGG | 20 | 7112 |
| HSV1-UL54-350 | + | AUUUGCCGUGCACGUACGGG | 20 | 7113 |
| HSV1-UL54-351 | + | UUUGCCGUGCACGUACGGGG | 20 | 7114 |
| HSV1-UL54-352 | + | CGGGGGGGCGACGAUGUGAC | 20 | 7115 |
| HSV1-UL54-353 | + | AGACACGACUCGAACACUCC | 20 | 7116 |
| HSV1-UL54-354 | + | CACGACUCGAACACUCCUGG | 20 | 7117 |
| HSV1-UL54-355 | + | ACUCCUGGCGGUGCGUGUCU | 20 | 7118 |
| HSV1-UL54-356 | + | GCGUGUCUAGGAUUUCGAUC | 20 | 7119 |
| HSV1-UL54-357 | + | UUCGAUCAGGCCCGCCAUGC | 20 | 7120 |
| HSV1-UL54-358 | + | CAGGCCCGCCAUGCAGGCCC | 20 | 7121 |
| HSV1-UL54-359 | + | AGGCCCGCCAUGCAGGCCCC | 20 | 7122 |
| HSV1-UL54-360 | + | GGCCCGCCAUGCAGGCCCCG | 20 | 7123 |
| HSV1-UL54-361 | + | CCGCCAUGCAGGCCCCGGGG | 20 | 7124 |
| HSV1-UL54-362 | + | UCUCUCCGACCCCGACACCA | 20 | 7125 |
| HSV1-UL54-363 | + | CUCUCCGACCCCGACACCAA | 20 | 7126 |
| HSV1-UL54-364 | + | CCGCGACGCCACGCUCGACG | 20 | 7127 |
| HSV1-UL54-365 | + | GACGCCACGCUCGACGCGGU | 20 | 7128 |
| HSV1-UL54-366 | + | CUCGACGCGGUUGGCGAGCC | 20 | 7129 |
| HSV1-UL54-367 | + | GGCCAGAAUGACAAACACGA | 20 | 7130 |
| HSV1-UL54-368 | + | GCCGCGAACACAGUUCGUCC | 20 | 7131 |
| HSV1-UL54-369 | + | ACAGUUCGUCCAGGCCGCAC | 20 | 7132 |
| HSV1-UL54-370 | + | CGCACAGGCCUCGCGCCUUC | 20 | 7133 |
| HSV1-UL54-371 | + | CUCGCGCCUUCAGGUAGCAC | 20 | 7134 |
| HSV1-UL54-372 | + | CUUCAGGUAGCACUGGAGAA | 20 | 7135 |
| HSV1-UL54-373 | + | UUCAGGUAGCACUGGAGAAA | 20 | 7136 |
| HSV1-UL54-374 | + | AGCACUGGAGAAAGGGCCGC | 20 | 7137 |
| HSV1-UL54-375 | + | GAGAAAGGGCCGCAGGCGCG | 20 | 7138 |
| HSV1-UL54-376 | + | AGGGCCGCAGGCGCGUGGCG | 20 | 7139 |
| HSV1-UL54-377 | + | GAGGUUAUCCAGCACAGCCG | 20 | 7140 |
| HSV1-UL54-378 | + | AGCCGCGGUCGUCCCGAUAA | 20 | 7141 |
| HSV1-UL54-379 | + | GCCGCGGUCGUCCCGAUAAU | 20 | 7142 |
| HSV1-UL54-380 | + | CCGCGGUCGUCCCGAUAAUG | 20 | 7143 |
| HSV1-UL54-381 | + | UCGUCCCGAUAAUGGGGUCC | 20 | 7144 |
| HSV1-UL54-382 | + | CGUCCCGAUAAUGGGGUCCU | 20 | 7145 |
| HSV1-UL54-383 | + | GUCCCGAUAAUGGGGUCCUG | 20 | 7146 |
| HSV1-UL54-384 | + | UCCCGAUAAUGGGGUCCUGG | 20 | 7147 |
| HSV1-UL54-385 | + | AUGGGGUCCUGGGGGCGCAG | 20 | 7148 |
| HSV1-UL54-386 | + | GGUCCUGGGGGCGCAGCGGC | 20 | 7149 |
| HSV1-UL54-387 | + | GGGGGCGCAGCGGCAGGUUG | 20 | 7150 |
| HSV1-UL54-388 | + | GGCGCAGCGGCAGGUUGUGG | 20 | 7151 |
| HSV1-UL54-389 | + | GCACCACGCCAGCGUCUCGU | 20 | 7152 |
| HSV1-UL54-390 | + | CCACGCCAGCGUCUCGUCGG | 20 | 7153 |
| HSV1-UL54-391 | + | CGCCAGCGUCUCGUCGGCGG | 20 | 7154 |
| HSV1-UL54-392 | + | GCGCCUCGAUGAAAUUUUCU | 20 | 7155 |
| HSV1-UL54-393 | + | GCGCAGCACGCAGUCGCGCA | 20 | 7156 |
| HSV1-UL54-394 | + | CACGCAGUCGCGCAUGGCCU | 20 | 7157 |
| HSV1-UL54-395 | + | GCAGUCGCGCAUGGCCUUGG | 20 | 7158 |
| HSV1-UL54-396 | + | CAUGGCCUUGGCGGUCGAUG | 20 | 7159 |
| HSV1-UL54-397 | + | UUGGCGGUCGAUGCGGCCCG | 20 | 7160 |
| HSV1-UL54-398 | + | CGAUGCGGCCCGAGGAUUGC | 20 | 7161 |
| HSV1-UL54-399 | + | UGCCGGCAAAAGUGCGAUAG | 20 | 7162 |
| HSV1-UL54-400 | + | GCAAAAGUGCGAUAGAGGCU | 20 | 7163 |
| HSV1-UL54-401 | + | CAAAAGUGCGAUAGAGGCUC | 20 | 7164 |
| HSV1-UL54-402 | + | UGCGAUAGAGGCUCGGGCCG | 20 | 7165 |
| HSV1-UL54-403 | + | GCGAUAGAGGCUCGGGCCGU | 20 | 7166 |
| HSV1-UL54-404 | + | GCUCGGGCCGUGGGCGACCA | 20 | 7167 |
| HSV1-UL54-405 | + | GUGGGCGACCAAGGUUUCCC | 20 | 7168 |
| HSV1-UL54-406 | + | GGUUUCCCAGGAGACCCGUC | 20 | 7169 |
| HSV1-UL54-407 | + | CCAGGAGACCCGUCUGGUCU | 20 | 7170 |
| HSV1-UL54-408 | + | CCGUCUGGUCUCGGCGUCAA | 20 | 7171 |
| HSV1-UL54-409 | + | CGUCUGGUCUCGGCGUCAAA | 20 | 7172 |
| HSV1-UL54-410 | + | CGGCGUCAAAGGGCCCUCCU | 20 | 7173 |
| HSV1-UL54-411 | + | CCUCCUUGGCCCGCCAGCAC | 20 | 7174 |
| HSV1-UL54-412 | + | CUCCUUGGCCCGCCAGCACC | 20 | 7175 |
| HSV1-UL54-413 | + | UCCUUGGCCCGCCAGCACCG | 20 | 7176 |
| HSV1-UL54-414 | + | GCCCGCCAGCACCGGGGCCC | 20 | 7177 |
| HSV1-UL54-415 | + | CCCGCCAGCACCGGGGCCCA | 20 | 7178 |
| HSV1-UL54-416 | + | CCGCCAGCACCGGGGCCCAG | 20 | 7179 |
| HSV1-UL54-417 | + | CGGGGCCCAGGGGCUAUUCG | 20 | 7180 |
| HSV1-UL54-418 | + | GGCCCAGGGGCUAUUCGCGG | 20 | 7181 |
| HSV1-UL54-419 | + | GCCCAGGGGCUAUUCGCGGC | 20 | 7182 |
| HSV1-UL54-420 | + | GGGCUAUUCGCGGCGGGAAA | 20 | 7183 |
| HSV1-UL54-421 | + | GGGAAACGGCUGCCCCCCAA | 20 | 7184 |
| HSV1-UL54-422 | + | GGAAACGGCUGCCCCCCAAA | 20 | 7185 |
| HSV1-UL54-423 | + | GAAACGGCUGCCCCCCAAAG | 20 | 7186 |
| HSV1-UL54-424 | + | CGUGCAUGACCUGUGCGCUG | 20 | 7187 |
| HSV1-UL54-425 | + | GGCCAAAGCUCUCGCUGAUG | 20 | 7188 |
| HSV1-UL54-426 | + | GCGGUCGACCGCCGCGCGCU | 20 | 7189 |
| HSV1-UL54-427 | + | GACCGCCGCGCGCUCGGAGA | 20 | 7190 |
| HSV1-UL54-428 | + | CGCGCUCGGAGAUGGAGCGC | 20 | 7191 |
| HSV1-UL54-429 | + | GGAGCGCAGGACCAACCGCG | 20 | 7192 |
| HSV1-UL54-430 | + | GCGCAGGACCAACCGCGUGG | 20 | 7193 |
| HSV1-UL54-431 | + | CAACCGCGUGGUGGCGUCGA | 20 | 7194 |
| HSV1-UL54-432 | + | CGUGGUGGCGUCGAUGGUGU | 20 | 7195 |
| HSV1-UL54-433 | + | GGUGGCGUCGAUGGUGUCGG | 20 | 7196 |
| HSV1-UL54-434 | + | GGCGUCGAUGGUGUCGGCGG | 20 | 7197 |
| HSV1-UL54-435 | + | GCGUCGAUGGUGUCGGCGGC | 20 | 7198 |
| HSV1-UL54-436 | + | GCGGCGGGCGCCUUUCGCUC | 20 | 7199 |
| HSV1-UL54-437 | + | CGGCGGGCGCCUUUCGCUCC | 20 | 7200 |
| HSV1-UL54-438 | + | GGCGGGCGCCUUUCGCUCCG | 20 | 7201 |
| HSV1-UL54-439 | + | GGCGCCUUUCGCUCCGGGGC | 20 | 7202 |
| HSV1-UL54-440 | + | GCGCCUUUCGCUCCGGGGCC | 20 | 7203 |
| HSV1-UL54-441 | + | CGCCUUUCGCUCCGGGGCCG | 20 | 7204 |
| HSV1-UL54-442 | + | UUCGCUCCGGGGCCGGGGCG | 20 | 7205 |
| HSV1-UL54-443 | + | UCGCUCCGGGGCCGGGGCGC | 20 | 7206 |
| HSV1-UL54-444 | + | CGCUCCGGGGCCGGGGCGCG | 20 | 7207 |
| HSV1-UL54-445 | + | GCUCCGGGGCCGGGGCGCGG | 20 | 7208 |
| HSV1-UL54-446 | + | GGCCGGGGCGCGGGGGUCCG | 20 | 7209 |
| HSV1-UL54-447 | + | GCCGGGGCGCGGGGGUCCGC | 20 | 7210 |
| HSV1-UL54-448 | + | GGGGCGCGGGGGUCCGCGGG | 20 | 7211 |
| HSV1-UL54-449 | + | GGGCGCGGGGGUCCGCGGGC | 20 | 7212 |
| HSV1-UL54-450 | + | GGCGCGGGGGUCCGCGGGCG | 20 | 7213 |
| HSV1-UL54-451 | + | GCGCGGGGGUCCGCGGGCGG | 20 | 7214 |
| HSV1-UL54-452 | + | CGCGGGGGUCCGCGGGCGGG | 20 | 7215 |
| HSV1-UL54-453 | + | GCGGGGGUCCGCGGGCGGGG | 20 | 7216 |
| HSV1-UL54-454 | + | GCAAUCGCCAGCGUCAUUAG | 20 | 7217 |
| HSV1-UL54-455 | + | CAAUCGCCAGCGUCAUUAGC | 20 | 7218 |
| HSV1-UL54-456 | + | AAUCGCCAGCGUCAUUAGCG | 20 | 7219 |
| HSV1-UL54-457 | + | AUCGCCAGCGUCAUUAGCGG | 20 | 7220 |
| HSV1-UL54-458 | + | UCGCCAGCGUCAUUAGCGGG | 20 | 7221 |
| HSV1-UL54-459 | + | CGCCAGCGUCAUUAGCGGGG | 20 | 7222 |
| HSV1-UL54-460 | + | GCCAGCGUCAUUAGCGGGGG | 20 | 7223 |
| HSV1-UL54-461 | + | UCAUUAGCGGGGGGGGUGCU | 20 | 7224 |
| HSV1-UL54-462 | + | GGCGCACGCCCCGUGUCCGC | 20 | 7225 |
| HSV1-UL54-463 | + | GCCCCGUGUCCGCUGGCCUC | 20 | 7226 |
| HSV1-UL54-464 | + | CCCCGUGUCCGCUGGCCUCC | 20 | 7227 |
| HSV1-UL54-465 | + | CGUGUCCGCUGGCCUCCGGG | 20 | 7228 |
| HSV1-UL54-466 | + | GUGUCCGCUGGCCUCCGGGU | 20 | 7229 |
| HSV1-UL54-467 | + | CCGCUGGCCUCCGGGUGGGU | 20 | 7230 |
| HSV1-UL54-468 | + | CGCUGGCCUCCGGGUGGGUC | 20 | 7231 |
| HSV1-UL54-469 | + | UGCCGCGCCACGCCUCGCCA | 20 | 7232 |
| HSV1-UL54-470 | + | GCCGCGCCACGCCUCGCCAU | 20 | 7233 |
| HSV1-UL54-471 | + | CCGCGCCACGCCUCGCCAUG | 20 | 7234 |
| HSV1-UL54-472 | + | CGCGCCACGCCUCGCCAUGG | 20 | 7235 |
| HSV1-UL54-473 | + | GCGCCACGCCUCGCCAUGGG | 20 | 7236 |
| HSV1-UL54-474 | + | CGCCUCGCCAUGGGGGGCGC | 20 | 7237 |
| HSV1-UL54-475 | + | GCCUCGCCAUGGGGGGCGCC | 20 | 7238 |
| HSV1-UL54-476 | + | CCUCGCCAUGGGGGGCGCCG | 20 | 7239 |
| HSV1-UL54-477 | + | GCCGUCCGUCCACCCCGCCC | 20 | 7240 |
| HSV1-UL54-478 | + | CCGUCCGUCCACCCCGCCCC | 20 | 7241 |
| HSV1-UL54-479 | + | CGUCCGUCCACCCCGCCCCG | 20 | 7242 |
| HSV1-UL54-480 | + | CCGUCCACCCCGCCCCGGGG | 20 | 7243 |
| HSV1-UL54-481 | + | CGUCCACCCCGCCCCGGGGC | 20 | 7244 |
| HSV1-UL54-482 | + | GUCCACCCCGCCCCGGGGCG | 20 | 7245 |
| HSV1-UL54-483 | + | GCCCCGGGGCGGGGUCCCCC | 20 | 7246 |
| HSV1-UL54-484 | + | CCCCGGGGCGGGGUCCCCCA | 20 | 7247 |
| HSV1-UL54-485 | + | GGGUCCCCCAGGGUUGCGAU | 20 | 7248 |
| HSV1-UL54-486 | − | AGAACCAAUCGCAACCC | 17 | 7249 |
| HSV1-UL54-487 | − | GAACCAAUCGCAACCCU | 17 | 7250 |
| HSV1-UL54-488 | − | AACCAAUCGCAACCCUG | 17 | 7251 |
| HSV1-UL54-489 | − | ACCAAUCGCAACCCUGG | 17 | 7252 |
| HSV1-UL54-490 | − | CCUGGGGGACCCCGCCC | 17 | 7253 |
| HSV1-UL54-491 | − | CUGGGGGACCCCGCCCC | 17 | 7254 |
| HSV1-UL54-492 | − | UGGGGGACCCCGCCCCG | 17 | 7255 |
| HSV1-UL54-493 | − | GGGACCCCGCCCCGGGG | 17 | 7256 |
| HSV1-UL54-494 | − | GGACCCCGCCCCGGGGC | 17 | 7257 |
| HSV1-UL54-495 | − | GACCCCGCCCCGGGGCG | 17 | 7258 |
| HSV1-UL54-496 | − | CCCGCCCCGGGGCGGGG | 17 | 7259 |
| HSV1-UL54-497 | − | CCCCGGGGCGGGGUGGA | 17 | 7260 |
| HSV1-UL54-498 | − | GGGGCGGGGUGGACGGA | 17 | 7261 |
| HSV1-UL54-499 | − | GGGUGGACGGACGGCCC | 17 | 7262 |
| HSV1-UL54-500 | − | GGCCCCGGCGCCCCCCA | 17 | 7263 |
| HSV1-UL54-501 | − | CGGCGCCCCCCAUGGCG | 17 | 7264 |
| HSV1-UL54-502 | − | CCCCCCAUGGCGAGGCG | 17 | 7265 |
| HSV1-UL54-503 | − | CAUGGCGAGGCGUGGCG | 17 | 7266 |
| HSV1-UL54-504 | − | GAGCAGCCCGACCCACC | 17 | 7267 |
| HSV1-UL54-505 | − | CAGCCCGACCCACCCGG | 17 | 7268 |
| HSV1-UL54-506 | − | ACCCACCCGGAGGCCAG | 17 | 7269 |
| HSV1-UL54-507 | − | CCGGAGGCCAGCGGACA | 17 | 7270 |
| HSV1-UL54-508 | − | CGGAGGCCAGCGGACAC | 17 | 7271 |
| HSV1-UL54-509 | − | GGAGGCCAGCGGACACG | 17 | 7272 |
| HSV1-UL54-510 | − | CCCCCCGCUAAUGACGC | 17 | 7273 |
| HSV1-UL54-511 | − | GAUUGCCCCCCCGCCCG | 17 | 7274 |
| HSV1-UL54-512 | − | CGCGGACCCCCGCGCCC | 17 | 7275 |
| HSV1-UL54-513 | − | CCCCCGCGCCCCGGCCC | 17 | 7276 |
| HSV1-UL54-514 | − | CCCGGCCCCGGAGCGAA | 17 | 7277 |
| HSV1-UL54-515 | − | CCAUCGACGCCACCACG | 17 | 7278 |
| HSV1-UL54-516 | − | CGACGCCACCACGCGGU | 17 | 7279 |
| HSV1-UL54-517 | − | CUCCAUCUCCGAGCGCG | 17 | 7280 |
| HSV1-UL54-518 | − | CAUCUCCGAGCGCGCGG | 17 | 7281 |
| HSV1-UL54-519 | − | CGCAUCAGCGAGAGCUU | 17 | 7282 |
| HSV1-UL54-520 | − | CUUUGGCCGCAGCGCAC | 17 | 7283 |
| HSV1-UL54-521 | − | GUCAUGCACGACCCCUU | 17 | 7284 |
| HSV1-UL54-522 | − | UCAUGCACGACCCCUUU | 17 | 7285 |
| HSV1-UL54-523 | − | CAUGCACGACCCCUUUG | 17 | 7286 |
| HSV1-UL54-524 | − | AUGCACGACCCCUUUGG | 17 | 7287 |
| HSV1-UL54-525 | − | UGCACGACCCCUUUGGG | 17 | 7288 |
| HSV1-UL54-526 | − | CCGCCGCGAAUAGCCCC | 17 | 7289 |
| HSV1-UL54-527 | − | CGCCGCGAAUAGCCCCU | 17 | 7290 |
| HSV1-UL54-528 | − | GAAUAGCCCCUGGGCCC | 17 | 7291 |
| HSV1-UL54-529 | − | CCCCUGGGCCCCGGUGC | 17 | 7292 |
| HSV1-UL54-530 | − | CUGGGCCCCGGUGCUGG | 17 | 7293 |
| HSV1-UL54-531 | − | UGGGCCCCGGUGCUGGC | 17 | 7294 |
| HSV1-UL54-532 | − | CCGGUGCUGGCGGGCCA | 17 | 7295 |
| HSV1-UL54-533 | − | GUGCUGGCGGGCCAAGG | 17 | 7296 |
| HSV1-UL54-534 | − | UGCUGGCGGGCCAAGGA | 17 | 7297 |
| HSV1-UL54-535 | − | UUGACGCCGAGACCAGA | 17 | 7298 |
| HSV1-UL54-536 | − | UGACGCCGAGACCAGAC | 17 | 7299 |
| HSV1-UL54-537 | − | AGACCAGACGGGUCUCC | 17 | 7300 |
| HSV1-UL54-538 | − | GACCAGACGGGUCUCCU | 17 | 7301 |
| HSV1-UL54-539 | − | GGUCUCCUGGGAAACCU | 17 | 7302 |
| HSV1-UL54-540 | − | GAAACCUUGGUCGCCCA | 17 | 7303 |
| HSV1-UL54-541 | − | CUCUAUCGCACUUUUGC | 17 | 7304 |
| HSV1-UL54-542 | − | CUUUUGCCGGCAAUCCU | 17 | 7305 |
| HSV1-UL54-543 | − | UUUUGCCGGCAAUCCUC | 17 | 7306 |
| HSV1-UL54-544 | − | GGCCGCAUCGACCGCCA | 17 | 7307 |
| HSV1-UL54-545 | − | CCAAGAAAAUUUCAUCG | 17 | 7308 |
| HSV1-UL54-546 | − | AAAUUUCAUCGAGGCGC | 17 | 7309 |
| HSV1-UL54-547 | − | CUCCGCCGACGAGACGC | 17 | 7310 |
| HSV1-UL54-548 | − | CCGACGAGACGCUGGCG | 17 | 7311 |
| HSV1-UL54-549 | − | CCUGCCGCUGCGCCCCC | 17 | 7312 |
| HSV1-UL54-550 | − | CCCCAGGACCCCAUUAU | 17 | 7313 |
| HSV1-UL54-551 | − | CCCAGGACCCCAUUAUC | 17 | 7314 |
| HSV1-UL54-552 | − | CAUUAUCGGGACGACCG | 17 | 7315 |
| HSV1-UL54-553 | − | GACGACCGCGGCUGUGC | 17 | 7316 |
| HSV1-UL54-554 | − | ACCUCGCCACGCGCCUG | 17 | 7317 |
| HSV1-UL54-555 | − | UCUCCAGUGCUACCUGA | 17 | 7318 |
| HSV1-UL54-556 | − | UGCUACCUGAAGGCGCG | 17 | 7319 |
| HSV1-UL54-557 | − | AAGGCGCGAGGCCUGUG | 17 | 7320 |
| HSV1-UL54-558 | − | GCGAGGCCUGUGCGGCC | 17 | 7321 |
| HSV1-UL54-559 | − | UGGACGAACUGUGUUCG | 17 | 7322 |
| HSV1-UL54-560 | − | ACGAACUGUGUUCGCGG | 17 | 7323 |
| HSV1-UL54-561 | − | GUGUUCGCGGCGGCGUC | 17 | 7324 |
| HSV1-UL54-562 | − | UUCGCGGCGGCGUCUGG | 17 | 7325 |
| HSV1-UL54-563 | − | GCGUCUGGCGGACAUUA | 17 | 7326 |
| HSV1-UL54-564 | − | CUUCGUGUUUGUCAUUC | 17 | 7327 |
| HSV1-UL54-565 | − | UGUUUGUCAUUCUGGCC | 17 | 7328 |
| HSV1-UL54-566 | − | GCCAACCGCGUCGAGCG | 17 | 7329 |
| HSV1-UL54-567 | − | CGUCGAGCGUGGCGUCG | 17 | 7330 |
| HSV1-UL54-568 | − | AUCGACUACGCGACCCU | 17 | 7331 |
| HSV1-UL54-569 | − | UACGCGACCCUUGGUGU | 17 | 7332 |
| HSV1-UL54-570 | − | ACGCGACCCUUGGUGUC | 17 | 7333 |
| HSV1-UL54-571 | − | CGCGACCCUUGGUGUCG | 17 | 7334 |
| HSV1-UL54-572 | − | ACCCUUGGUGUCGGGGU | 17 | 7335 |
| HSV1-UL54-573 | − | AUGCAUUUCUACCUCCC | 17 | 7336 |
| HSV1-UL54-574 | − | UGCAUUUCUACCUCCCC | 17 | 7337 |
| HSV1-UL54-575 | − | GCAUUUCUACCUCCCCG | 17 | 7338 |
| HSV1-UL54-576 | − | CCUCCCCGGGGCCUGCA | 17 | 7339 |
| HSV1-UL54-577 | − | CCCCGGGGCCUGCAUGG | 17 | 7340 |
| HSV1-UL54-578 | − | CCCGGGGCCUGCAUGGC | 17 | 7341 |
| HSV1-UL54-579 | − | CCUAGACACGCACCGCC | 17 | 7342 |
| HSV1-UL54-580 | − | UCGUGUCUGCGAGUUGA | 17 | 7343 |
| HSV1-UL54-581 | − | GCCCCCCCGUACGUGCA | 17 | 7344 |
| HSV1-UL54-582 | + | UAUUUGCCGUGCACGUA | 17 | 7345 |
| HSV1-UL54-583 | + | AUUUGCCGUGCACGUAC | 17 | 7346 |
| HSV1-UL54-584 | + | UUUGCCGUGCACGUACG | 17 | 7347 |
| HSV1-UL54-585 | + | UUGCCGUGCACGUACGG | 17 | 7348 |
| HSV1-UL54-586 | + | UGCCGUGCACGUACGGG | 17 | 7349 |
| HSV1-UL54-587 | + | GCCGUGCACGUACGGGG | 17 | 7350 |
| HSV1-UL54-588 | + | GGGGGCGACGAUGUGAC | 17 | 7351 |
| HSV1-UL54-589 | + | CACGACUCGAACACUCC | 17 | 7352 |
| HSV1-UL54-590 | + | GACUCGAACACUCCUGG | 17 | 7353 |
| HSV1-UL54-591 | + | CCUGGCGGUGCGUGUCU | 17 | 7354 |
| HSV1-UL54-592 | + | UGUCUAGGAUUUCGAUC | 17 | 7355 |
| HSV1-UL54-593 | + | GAUCAGGCCCGCCAUGC | 17 | 7356 |
| HSV1-UL54-594 | + | GCCCGCCAUGCAGGCCC | 17 | 7357 |
| HSV1-UL54-595 | + | CCCGCCAUGCAGGCCCC | 17 | 7358 |
| HSV1-UL54-596 | + | CCGCCAUGCAGGCCCCG | 17 | 7359 |
| HSV1-UL54-597 | + | CCAUGCAGGCCCCGGGG | 17 | 7360 |
| HSV1-UL54-598 | + | CUCCGACCCCGACACCA | 17 | 7361 |
| HSV1-UL54-599 | + | UCCGACCCCGACACCAA | 17 | 7362 |
| HSV1-UL54-600 | + | CGACGCCACGCUCGACG | 17 | 7363 |
| HSV1-UL54-601 | + | GCCACGCUCGACGCGGU | 17 | 7364 |
| HSV1-UL54-602 | + | GACGCGGUUGGCGAGCC | 17 | 7365 |
| HSV1-UL54-603 | + | CAGAAUGACAAACACGA | 17 | 7366 |
| HSV1-UL54-604 | + | GCGAACACAGUUCGUCC | 17 | 7367 |
| HSV1-UL54-605 | + | GUUCGUCCAGGCCGCAC | 17 | 7368 |
| HSV1-UL54-606 | + | ACAGGCCUCGCGCCUUC | 17 | 7369 |
| HSV1-UL54-607 | + | GCGCCUUCAGGUAGCAC | 17 | 7370 |
| HSV1-UL54-608 | + | CAGGUAGCACUGGAGAA | 17 | 7371 |
| HSV1-UL54-609 | + | AGGUAGCACUGGAGAAA | 17 | 7372 |
| HSV1-UL54-610 | + | ACUGGAGAAAGGGCCGC | 17 | 7373 |
| HSV1-UL54-611 | + | AAAGGGCCGCAGGCGCG | 17 | 7374 |
| HSV1-UL54-612 | + | GCCGCAGGCGCGUGGCG | 17 | 7375 |
| HSV1-UL54-613 | + | GUUAUCCAGCACAGCCG | 17 | 7376 |
| HSV1-UL54-614 | + | CGCGGUCGUCCCGAUAA | 17 | 7377 |
| HSV1-UL54-615 | + | GCGGUCGUCCCGAUAAU | 17 | 7378 |
| HSV1-UL54-616 | + | CGGUCGUCCCGAUAAUG | 17 | 7379 |
| HSV1-UL54-617 | + | UCCCGAUAAUGGGGUCC | 17 | 7380 |
| HSV1-UL54-618 | + | CCCGAUAAUGGGGUCCU | 17 | 7381 |
| HSV1-UL54-619 | + | CCGAUAAUGGGGUCCUG | 17 | 7382 |
| HSV1-UL54-620 | + | CGAUAAUGGGGUCCUGG | 17 | 7383 |
| HSV1-UL54-621 | + | GGGUCCUGGGGGCGCAG | 17 | 7384 |
| HSV1-UL54-622 | + | CCUGGGGGCGCAGCGGC | 17 | 7385 |
| HSV1-UL54-623 | + | GGCGCAGCGGCAGGUUG | 17 | 7386 |
| HSV1-UL54-624 | + | GCAGCGGCAGGUUGUGG | 17 | 7387 |
| HSV1-UL54-625 | + | CCACGCCAGCGUCUCGU | 17 | 7388 |
| HSV1-UL54-626 | + | CGCCAGCGUCUCGUCGG | 17 | 7389 |
| HSV1-UL54-627 | + | CAGCGUCUCGUCGGCGG | 17 | 7390 |
| HSV1-UL54-628 | + | CCUCGAUGAAAUUUUCU | 17 | 7391 |
| HSV1-UL54-629 | + | CAGCACGCAGUCGCGCA | 17 | 7392 |
| HSV1-UL54-630 | + | GCAGUCGCGCAUGGCCU | 17 | 7393 |
| HSV1-UL54-631 | + | GUCGCGCAUGGCCUUGG | 17 | 7394 |
| HSV1-UL54-632 | + | GGCCUUGGCGGUCGAUG | 17 | 7395 |
| HSV1-UL54-633 | + | GCGGUCGAUGCGGCCCG | 17 | 7396 |
| HSV1-UL54-634 | + | UGCGGCCCGAGGAUUGC | 17 | 7397 |
| HSV1-UL54-635 | + | CGGCAAAAGUGCGAUAG | 17 | 7398 |
| HSV1-UL54-636 | + | AAAGUGCGAUAGAGGCU | 17 | 7399 |
| HSV1-UL54-637 | + | AAGUGCGAUAGAGGCUC | 17 | 7400 |
| HSV1-UL54-638 | + | GAUAGAGGCUCGGGCCG | 17 | 7401 |
| HSV1-UL54-639 | + | AUAGAGGCUCGGGCCGU | 17 | 7402 |
| HSV1-UL54-640 | + | CGGGCCGUGGGCGACCA | 17 | 7403 |
| HSV1-UL54-641 | + | GGCGACCAAGGUUUCCC | 17 | 7404 |
| HSV1-UL54-642 | + | UUCCCAGGAGACCCGUC | 17 | 7405 |
| HSV1-UL54-643 | + | GGAGACCCGUCUGGUCU | 17 | 7406 |
| HSV1-UL54-644 | + | UCUGGUCUCGGCGUCAA | 17 | 7407 |
| HSV1-UL54-645 | + | CUGGUCUCGGCGUCAAA | 17 | 7408 |
| HSV1-UL54-646 | + | CGUCAAAGGGCCCUCCU | 17 | 7409 |
| HSV1-UL54-647 | + | CCUUGGCCCGCCAGCAC | 17 | 7410 |
| HSV1-UL54-648 | + | CUUGGCCCGCCAGCACC | 17 | 7411 |
| HSV1-UL54-649 | + | UUGGCCCGCCAGCACCG | 17 | 7412 |
| HSV1-UL54-650 | + | CGCCAGCACCGGGGCCC | 17 | 7413 |
| HSV1-UL54-651 | + | GCCAGCACCGGGGCCCA | 17 | 7414 |
| HSV1-UL54-652 | + | CCAGCACCGGGGCCCAG | 17 | 7415 |
| HSV1-UL54-653 | + | GGCCCAGGGGCUAUUCG | 17 | 7416 |
| HSV1-UL54-654 | + | CCAGGGGCUAUUCGCGG | 17 | 7417 |
| HSV1-UL54-655 | + | CAGGGGCUAUUCGCGGC | 17 | 7418 |
| HSV1-UL54-656 | + | CUAUUCGCGGCGGGAAA | 17 | 7419 |
| HSV1-UL54-657 | + | AAACGGCUGCCCCCCAA | 17 | 7420 |
| HSV1-UL54-658 | + | AACGGCUGCCCCCCAAA | 17 | 7421 |
| HSV1-UL54-659 | + | ACGGCUGCCCCCCAAAG | 17 | 7422 |
| HSV1-UL54-660 | + | GCAUGACCUGUGCGCUG | 17 | 7423 |
| HSV1-UL54-661 | + | CAAAGCUCUCGCUGAUG | 17 | 7424 |
| HSV1-UL54-662 | + | GUCGACCGCCGCGCGCU | 17 | 7425 |
| HSV1-UL54-663 | + | CGCCGCGCGCUCGGAGA | 17 | 7426 |
| HSV1-UL54-664 | + | GCUCGGAGAUGGAGCGC | 17 | 7427 |
| HSV1-UL54-665 | + | GCGCAGGACCAACCGCG | 17 | 7428 |
| HSV1-UL54-666 | + | CAGGACCAACCGCGUGG | 17 | 7429 |
| HSV1-UL54-667 | + | CCGCGUGGUGGCGUCGA | 17 | 7430 |
| HSV1-UL54-668 | + | GGUGGCGUCGAUGGUGU | 17 | 7431 |
| HSV1-UL54-669 | + | GGCGUCGAUGGUGUCGG | 17 | 7432 |
| HSV1-UL54-670 | + | GUCGAUGGUGUCGGCGG | 17 | 7433 |
| HSV1-UL54-671 | + | UCGAUGGUGUCGGCGGC | 17 | 7434 |
| HSV1-UL54-672 | + | GCGGGCGCCUUUCGCUC | 17 | 7435 |
| HSV1-UL54-673 | + | CGGGCGCCUUUCGCUCC | 17 | 7436 |
| HSV1-UL54-674 | + | GGGCGCCUUUCGCUCCG | 17 | 7437 |
| HSV1-UL54-675 | + | GCCUUUCGCUCCGGGGC | 17 | 7438 |
| HSV1-UL54-676 | + | CCUUUCGCUCCGGGGCC | 17 | 7439 |
| HSV1-UL54-677 | + | CUUUCGCUCCGGGGCCG | 17 | 7440 |
| HSV1-UL54-678 | + | GCUCCGGGGCCGGGGCG | 17 | 7441 |
| HSV1-UL54-679 | + | CUCCGGGGCCGGGGCGC | 17 | 7442 |
| HSV1-UL54-680 | + | UCCGGGGCCGGGGCGCG | 17 | 7443 |
| HSV1-UL54-681 | + | CCGGGGCCGGGGCGCGG | 17 | 7444 |
| HSV1-UL54-682 | + | CGGGGCGCGGGGGUCCG | 17 | 7445 |
| HSV1-UL54-683 | + | GGGGCGCGGGGGUCCGC | 17 | 7446 |
| HSV1-UL54-684 | + | GCGCGGGGGUCCGCGGG | 17 | 7447 |
| HSV1-UL54-685 | + | CGCGGGGGUCCGCGGGC | 17 | 7448 |
| HSV1-UL54-686 | + | GCGGGGGUCCGCGGGCG | 17 | 7449 |
| HSV1-UL54-687 | + | CGGGGGUCCGCGGGCGG | 17 | 7450 |
| HSV1-UL54-688 | + | GGGGGUCCGCGGGCGGG | 17 | 7451 |
| HSV1-UL54-689 | + | GGGGUCCGCGGGCGGGG | 17 | 7452 |
| HSV1-UL54-690 | + | AUCGCCAGCGUCAUUAG | 17 | 7453 |
| HSV1-UL54-691 | + | UCGCCAGCGUCAUUAGC | 17 | 7454 |
| HSV1-UL54-692 | + | CGCCAGCGUCAUUAGCG | 17 | 7455 |
| HSV1-UL54-693 | + | GCCAGCGUCAUUAGCGG | 17 | 7456 |
| HSV1-UL54-694 | + | CCAGCGUCAUUAGCGGG | 17 | 7457 |
| HSV1-UL54-695 | + | CAGCGUCAUUAGCGGGG | 17 | 7458 |
| HSV1-UL54-696 | + | AGCGUCAUUAGCGGGGG | 17 | 7459 |
| HSV1-UL54-697 | + | UUAGCGGGGGGGGUGCU | 17 | 7460 |
| HSV1-UL54-698 | + | GCACGCCCCGUGUCCGC | 17 | 7461 |
| HSV1-UL54-699 | + | CCGUGUCCGCUGGCCUC | 17 | 7462 |
| HSV1-UL54-700 | + | CGUGUCCGCUGGCCUCC | 17 | 7463 |
| HSV1-UL54-701 | + | GUCCGCUGGCCUCCGGG | 17 | 7464 |
| HSV1-UL54-702 | + | UCCGCUGGCCUCCGGGU | 17 | 7465 |
| HSV1-UL54-703 | + | CUGGCCUCCGGGUGGGU | 17 | 7466 |
| HSV1-UL54-704 | + | UGGCCUCCGGGUGGGUC | 17 | 7467 |
| HSV1-UL54-705 | + | CGCGCCACGCCUCGCCA | 17 | 7468 |
| HSV1-UL54-706 | + | GCGCCACGCCUCGCCAU | 17 | 7469 |
| HSV1-UL54-707 | + | CGCCACGCCUCGCCAUG | 17 | 7470 |
| HSV1-UL54-708 | + | GCCACGCCUCGCCAUGG | 17 | 7471 |
| HSV1-UL54-709 | + | CCACGCCUCGCCAUGGG | 17 | 7472 |
| HSV1-UL54-710 | + | CUCGCCAUGGGGGGCGC | 17 | 7473 |
| HSV1-UL54-711 | + | UCGCCAUGGGGGGCGCC | 17 | 7474 |
| HSV1-UL54-712 | + | CGCCAUGGGGGGCGCCG | 17 | 7475 |
| HSV1-UL54-713 | + | GUCCGUCCACCCCGCCC | 17 | 7476 |
| HSV1-UL54-714 | + | UCCGUCCACCCCGCCCC | 17 | 7477 |
| HSV1-UL54-715 | + | CCGUCCACCCCGCCCCG | 17 | 7478 |
| HSV1-UL54-716 | + | UCCACCCCGCCCCGGGG | 17 | 7479 |
| HSV1-UL54-717 | + | CCACCCCGCCCCGGGGC | 17 | 7480 |
| HSV1-UL54-718 | + | CACCCCGCCCCGGGGCG | 17 | 7481 |
| HSV1-UL54-719 | + | CCGGGGCGGGGUCCCCC | 17 | 7482 |
| HSV1-UL54-720 | + | CGGGGCGGGGUCCCCCA | 17 | 7483 |
| HSV1-UL54-721 | + | UCCCCCAGGGUUGCGAU | 17 | 7484 |

Table 4D provides exemplary targeting domains for knocking out the UL54 gene selected according to the first tier parameters. The targeting domains are selected based on location within first 500 bp of the coding sequence of the UL54 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 4D
1st Tier

| | | | Target | |
|---|---|---|---|---|
| gRNA Name | DNA Strand | Targeting Domain | Site Length | Seq ID |
| HSV1-UL54-726 | − | CCUCUCCGACAGCGAUCUGG | 20 | 7487 |
| HSV1-UL54-727 | − | GGACAUGGAAGACCCCCACG | 20 | 7488 |
| HSV1-UL54-728 | − | GGAAGACCCCCACGGAGAGG | 20 | 7489 |
| HSV1-UL54-729 | − | ACGAGGACCCCCCCGAGCCG | 20 | 7490 |
| HSV1-UL54-730 | − | AGGACCCCCCCGAGCCGGCG | 20 | 7491 |
| HSV1-UL54-731 | − | CGGGGAGUGUUCCUCGUCGG | 20 | 7492 |
| HSV1-UL54-5 | − | UUCCUCGUCGGACGAGGACA | 20 | 6768 |
| HSV1-UL54-733 | − | AUAUGCUAAUUGACCUCGGC | 20 | 7493 |
| HSV1-UL54-734 | − | UGGACCUCUCCGACAGCGAU | 20 | 7494 |
| HSV1-UL54-735 | − | UCUCCGACAGCGAUCUGGAC | 20 | 7495 |
| HSV1-UL54-736 | − | CGAUCUGGACGAGGACCCCC | 20 | 7496 |
| HSV1-UL54-84 | − | CGAGGACCCCCCCGAGCCGG | 20 | 6847 |
| HSV1-UL54-738 | − | CGGAGAGCCGCCGCGACGAC | 20 | 7497 |
| HSV1-UL54-85 | − | GGAGAGCCGCCGCGACGACC | 20 | 6848 |
| HSV1-UL54-740 | − | GCCGCCGCGACGACCUGGAA | 20 | 7498 |
| HSV1-UL54-741 | − | CGACCUGGAAUCGGACAGCA | 20 | 7499 |
| HSV1-UL54-86 | − | GACCUGGAAUCGGACAGCAG | 20 | 6849 |
| HSV1-UL54-87 | − | ACCUGGAAUCGGACAGCAGC | 20 | 6850 |
| HSV1-UL54-88 | − | CCUGGAAUCGGACAGCAGCG | 20 | 6851 |
| HSV1-UL54-745 | − | GCAGCGGGGAGUGUUCCUCG | 20 | 7500 |
| HSV1-UL54-746 | − | GGGAGUGUUCCUCGUCGGAC | 20 | 7501 |
| HSV1-UL54-747 | − | GUUCCUCGUCGGACGAGGAC | 20 | 7502 |
| HSV1-UL54-748 | − | CGAGGACAUGGAAGACCCCC | 20 | 7503 |
| HSV1-UL54-90 | − | GAGGACAUGGAAGACCCCCA | 20 | 6853 |
| HSV1-UL54-750 | − | ACAUGGAAGACCCCCACGGA | 20 | 7504 |
| HSV1-UL54-751 | − | ACCCCCACGGAGAGGACGGA | 20 | 7505 |
| HSV1-UL54-93 | − | CCCCCACGGAGAGGACGGAC | 20 | 6856 |
| HSV1-UL54-753 | − | GGCGGUCCGCCCGUCUCGUC | 20 | 7506 |
| HSV1-UL54-754 | − | GCACCCAGACGCCUCGUCCG | 20 | 7507 |
| HSV1-UL54-8 | − | CACCCAGACGCCUCGUCCGA | 20 | 6771 |
| HSV1-UL54-756 | − | CGCCUCGUCCGACGGAGCGG | 20 | 7508 |
| HSV1-UL54-757 | − | CCACAGUGUGUGGUCGCGCC | 20 | 7509 |
| HSV1-UL54-98 | − | CACAGUGUGUGGUCGCGCCU | 20 | 6861 |
| HSV1-UL54-759 | − | CCGGCGACCGUCUUGCUCCC | 20 | 7510 |
| HSV1-UL54-760 | − | GUCUUGCUCCCCCGAGCAGC | 20 | 7511 |
| HSV1-UL54-99 | − | UCUUGCUCCCCCGAGCAGCA | 20 | 6862 |
| HSV1-UL54-100 | − | CUUGCUCCCCCGAGCAGCAC | 20 | 6863 |
| HSV1-UL54-763 | − | CAAAGCCCAGCCUGCCCGCG | 20 | 7512 |
| HSV1-UL54-764 | − | GCCUGCCCGCGGCGGACGCC | 20 | 7513 |
| HSV1-UL54-765 | − | GGCGGACGCCGUGGGCGUCG | 20 | 7514 |
| HSV1-UL54-108 | − | GCGGACGCCGUGGGCGUCGC | 20 | 6871 |
| HSV1-UL54-767 | − | CGCCGUGGGCGUCGCAGGGG | 20 | 7515 |
| HSV1-UL54-111 | − | GCCGUGGGCGUCGCAGGGGU | 20 | 6874 |
| HSV1-UL54-769 | − | GGGUCGGGGUCGCGGUGGUC | 20 | 7516 |
| HSV1-UL54-116 | − | GGUCGGGGUCGCGGUGGUCC | 20 | 6879 |
| HSV1-UL54-771 | − | CCGGGGCUGCCGAUGGUUUG | 20 | 7517 |
| HSV1-UL54-772 | + | GACCACCGCGACCCCGACCC | 20 | 7518 |
| HSV1-UL54-773 | + | GCGACGCCCACGGCGUCCGC | 20 | 7519 |
| HSV1-UL54-137 | + | UGCUGCUCGGGGGAGCAAGA | 20 | 6900 |
| HSV1-UL54-775 | + | GGCGCGACCACACACUGUGG | 20 | 7520 |
| HSV1-UL54-776 | + | CAAACCAUCGGCAGCCCCGG | 20 | 7521 |
| HSV1-UL54-777 | + | GGACCACCGCGACCCCGACC | 20 | 7522 |
| HSV1-UL54-778 | + | ACCCCUGCGACGCCCACGGC | 20 | 7523 |
| HSV1-UL54-779 | + | CCCCUGCGACGCCCACGGCG | 20 | 7524 |
| HSV1-UL54-780 | + | CGACGCCCACGGCGUCCGCC | 20 | 7525 |
| HSV1-UL54-123 | + | GGCGUCCGCCGCGGGCAGGC | 20 | 6886 |
| HSV1-UL54-782 | + | CGCGGGCAGGCUGGGCUUUG | 20 | 7526 |
| HSV1-UL54-783 | + | GUGCUGCUCGGGGGAGCAAG | 20 | 7527 |
| HSV1-UL54-784 | + | GCUGCUCGGGGGAGCAAGAC | 20 | 7528 |
| HSV1-UL54-785 | + | GGGAGCAAGACGGUCGCCGG | 20 | 7529 |
| HSV1-UL54-139 | + | AGGCGCGACCACACACUGUG | 20 | 6902 |
| HSV1-UL54-787 | + | CCGCUCCGUCGGACGAGGCG | 20 | 7530 |
| HSV1-UL54-788 | + | UCGGACGAGGCGUCUGGGUG | 20 | 7531 |
| HSV1-UL54-145 | + | CGGACGAGGCGUCUGGGUGC | 20 | 6908 |
| HSV1-UL54-154 | + | GACGAGACGGGCGGACCGCC | 20 | 6917 |
| HSV1-UL54-791 | + | GUCCGUCCUCUCCGUGGGGG | 20 | 7532 |
| HSV1-UL54-792 | + | UCCGUCCUCUCCGUGGGGGU | 20 | 7533 |
| HSV1-UL54-793 | + | CCUCUCCGUGGGGGUCUUCC | 20 | 7534 |
| HSV1-UL54-794 | + | UCCGUGGGGGUCUUCCAUGU | 20 | 7535 |
| HSV1-UL54-795 | + | CGUGGGGGUCUUCCAUGUCC | 20 | 7536 |
| HSV1-UL54-796 | + | UGGGGGUCUUCCAUGUCCUC | 20 | 7537 |
| HSV1-UL54-797 | + | GGGGGUCUUCCAUGUCCUCG | 20 | 7538 |
| HSV1-UL54-798 | + | UGUCCUCGUCCGACGAGGAA | 20 | 7539 |
| HSV1-UL54-799 | + | GUCCUCGUCCGACGAGGAAC | 20 | 7540 |
| HSV1-UL54-800 | + | GUCCGACGAGGAACACUCCC | 20 | 7541 |
| HSV1-UL54-801 | + | CCGACGAGGAACACUCCCCG | 20 | 7542 |
| HSV1-UL54-802 | + | CGAGGAACACUCCCCGCUGC | 20 | 7543 |
| HSV1-UL54-803 | + | CGCUGCUGUCCGAUUCCAGG | 20 | 7544 |
| HSV1-UL54-804 | + | GCUGCUGUCCGAUUCCAGGU | 20 | 7545 |
| HSV1-UL54-805 | + | CUGCUGUCCGAUUCCAGGUC | 20 | 7546 |
| HSV1-UL54-806 | + | UGCUGUCCGAUUCCAGGUCG | 20 | 7547 |
| HSV1-UL54-807 | + | UUCCAGGUCGUCGCGGCGGC | 20 | 7548 |
| HSV1-UL54-808 | + | GGUCGUCGCGGCGGCUCUCC | 20 | 7549 |
| HSV1-UL54-809 | + | GUCGUCGCGGCGGCUCUCCG | 20 | 7550 |
| HSV1-UL54-810 | + | CGCCGGCUCGGGGGGGUCCU | 20 | 7551 |
| HSV1-UL54-811 | + | CCGGCUCGGGGGGGUCCUCG | 20 | 7552 |
| HSV1-UL54-812 | + | CGGCUCGGGGGGGUCCUCGU | 20 | 7553 |
| HSV1-UL54-813 | + | GGGGGUCCUCGUCCAGAUCG | 20 | 7554 |
| HSV1-UL54-814 | + | GUCCAGAUCGCUGUCGGAGA | 20 | 7555 |
| HSV1-UL54-815 | + | CCAGAUCGCUGUCGGAGAGG | 20 | 7556 |
| HSV1-UL54-816 | + | AUCGCUGUCGGAGAGGUCCA | 20 | 7557 |
| HSV1-UL54-817 | + | GCCGAGGUCAAUUAGCAUAU | 20 | 7558 |
| HSV1-UL54-818 | − | CUCCGUGGGGGUCUUCCAUG | 17 | 7559 |
| HSV1-UL54-796 | − | UGGGGGUCUUCCAUGUCCUC | 20 | 7537 |
| HSV1-UL54-798 | − | UGUCCUCGUCCGACGAGGAA | 20 | 7539 |
| HSV1-UL54-821 | − | CGUCCGACGAGGAACACUCC | 20 | 7560 |
| HSV1-UL54-822 | − | ACGAGGAACACUCCCCGCUG | 20 | 7561 |
| HSV1-UL54-803 | − | CGCUGCUGUCCGAUUCCAGG | 20 | 7544 |
| HSV1-UL54-804 | + | GCUGCUGUCCGAUUCCAGGU | 20 | 7545 |
| HSV1-UL54-805 | + | CUGCUGUCCGAUUCCAGGUC | 20 | 7546 |
| HSV1-UL54-826 | + | AUUCCAGGUCGUCGCGGCGG | 20 | 7562 |
| HSV1-UL54-808 | + | GGUCGUCGCGGCGGCUCUCC | 20 | 7549 |
| HSV1-UL54-811 | + | CCGGCUCGGGGGGGUCCUCG | 20 | 7552 |
| HSV1-UL54-829 | + | GCUCGGGGGGGUCCUCGUCC | 20 | 7563 |
| HSV1-UL54-38 | + | CGUCCAGAUCGCUGUCGGAG | 20 | 6801 |
| HSV1-UL54-39 | + | GAUCGCUGUCGGAGAGGUCC | 20 | 6802 |
| HSV1-UL54-832 | + | GGCCGAGGUCAAUUAGCAUA | 20 | 7564 |
| HSV1-UL54-833 | − | AGGUCAAUUAGCAUAUCAAU | 20 | 7565 |
| HSV1-UL54-167 | − | GAUAUGCUAAUUGACCU | 17 | 6930 |
| HSV1-UL54-168 | − | GCUAAUUGACCUCGGCC | 17 | 6931 |
| HSV1-UL54-41 | − | CCUCUCCGACAGCGAUC | 17 | 6804 |
| HSV1-UL54-42 | − | CGACAGCGAUCUGGACG | 17 | 6805 |
| HSV1-UL54-169 | − | CGAGGACCCCCCCGAGC | 17 | 6932 |
| HSV1-UL54-170 | − | GGACCCCCCCGAGCCGG | 17 | 6933 |
| HSV1-UL54-171 | − | GAGCCGCCGCGACGACC | 17 | 6934 |
| HSV1-UL54-43 | − | CCGCGACGACCUGGAAU | 17 | 6806 |
| HSV1-UL54-172 | − | CUGGAAUCGGACAGCAG | 17 | 6935 |
| HSV1-UL54-173 | − | UGGAAUCGGACAGCAGC | 17 | 6936 |
| HSV1-UL54-174 | − | GGAAUCGGACAGCAGCG | 17 | 6937 |
| HSV1-UL54-44 | − | CGGGGAGUGUUCCUCGU | 17 | 6807 |
| HSV1-UL54-175 | − | GUGUUCCUCGUCGGACG | 17 | 6938 |
| HSV1-UL54-45 | − | CUCGUCGGACGAGGACA | 17 | 6808 |
| HSV1-UL54-176 | − | GACAUGGAAGACCCCCA | 17 | 6939 |
| HSV1-UL54-177 | − | GGAAGACCCCCACGGAG | 17 | 6940 |
| HSV1-UL54-178 | − | GACCCCCACGGAGAGGA | 17 | 6941 |
| HSV1-UL54-179 | − | CCACGGAGAGGACGGAC | 17 | 6942 |
| HSV1-UL54-46 | − | ACUCGACGCCGCUCGCC | 17 | 6809 |
| HSV1-UL54-47 | − | CGACGCCGCUCGCCCGG | 17 | 6810 |
| HSV1-UL54-180 | − | UCUCGUCCAGAAGACCC | 17 | 6943 |
| HSV1-UL54-48 | − | CCAGACGCCUCGUCCGA | 17 | 6811 |
| HSV1-UL54-181 | − | CGCCUCGUCCGACGGAG | 17 | 6944 |
| HSV1-UL54-49 | − | UCGUCCGACGGAGCGGC | 17 | 6812 |
| HSV1-UL54-182 | − | CGUCCGACGGAGCGGCA | 17 | 6945 |
| HSV1-UL54-183 | − | CAGCGCCCCACAGUGUG | 17 | 6946 |
| HSV1-UL54-184 | − | AGUGUGUGGUCGCGCCU | 17 | 6947 |
| HSV1-UL54-50 | − | GUGUGUGGUCGCGCCUC | 17 | 6813 |
| HSV1-UL54-51 | − | UGUGUGGUCGCGCCUCG | 17 | 6814 |
| HSV1-UL54-52 | − | GGUCGCGCCUCGGGGCC | 17 | 6815 |
| HSV1-UL54-185 | + | UGCUCCCCCGAGCAGCA | 17 | 6948 |
| HSV1-UL54-186 | + | GCUCCCCCGAGCAGCAC | 17 | 6949 |
| HSV1-UL54-53 | + | CUCCCCCGAGCAGCACG | 17 | 6816 |
| HSV1-UL54-187 | + | UCCCCCGAGCAGCACGG | 17 | 6950 |
| HSV1-UL54-188 | + | CGAGCAGCACGGGGGCA | 17 | 6951 |
| HSV1-UL54-189 | + | GCAGCACGGGGGCAAGG | 17 | 6952 |
| HSV1-UL54-190 | + | AAAGCCCAGCCUGCCCG | 17 | 6953 |
| HSV1-UL54-191 | + | GCCCAGCCUGCCCGCGG | 17 | 6954 |
| HSV1-UL54-192 | + | GCCCGCGGCGGACGCCG | 17 | 6955 |
| HSV1-UL54-193 | − | CCCGCGGCGGACGCCGU | 17 | 6956 |
| HSV1-UL54-194 | − | GACGCCGUGGGCGUCGC | 17 | 6957 |
| HSV1-UL54-195 | − | ACGCCGUGGGCGUCGCA | 17 | 6958 |
| HSV1-UL54-196 | − | CGCCGUGGGCGUCGCAG | 17 | 6959 |
| HSV1-UL54-197 | − | GUGGGCGUCGCAGGGGU | 17 | 6960 |
| HSV1-UL54-198 | − | UGGGCGUCGCAGGGGUC | 17 | 6961 |
| HSV1-UL54-199 | − | GGGCGUCGCAGGGGUCG | 17 | 6962 |
| HSV1-UL54-200 | − | CGCAGGGGUCGGGGUCG | 17 | 6963 |
| HSV1-UL54-201 | − | AGGGGUCGGGGUCGCGG | 17 | 6964 |
| HSV1-UL54-202 | − | CGGGGUCGCGGUGGUCC | 17 | 6965 |
| HSV1-UL54-203 | − | GGGGUCGCGGUGGUCCC | 17 | 6966 |
| HSV1-UL54-54 | − | GGGUCGCGGUGGUCCCG | 17 | 6817 |
| HSV1-UL54-204 | − | GGUCCCGGGGCUGCCGA | 17 | 6967 |
| HSV1-UL54-55 | − | GGCUGCCGAUGGUUUGU | 17 | 6818 |
| HSV1-UL54-56 | − | GUUUGUCGGACCCCCGC | 17 | 6819 |
| HSV1-UL54-888 | + | GCGGGGGUCCGACAAAC | 17 | 7566 |
| HSV1-UL54-889 | + | ACAAACCAUCGGCAGCC | 17 | 7567 |
| HSV1-UL54-890 | + | UCGGCAGCCCCGGGACC | 17 | 7568 |
| HSV1-UL54-891 | + | CGGGACCACCGCGACCC | 17 | 7569 |
| HSV1-UL54-892 | + | GGGACCACCGCGACCCC | 17 | 7570 |
| HSV1-UL54-893 | + | GGACCACCGCGACCCCG | 17 | 7571 |
| HSV1-UL54-894 | + | CCGCGACCCCGACCCCU | 17 | 7572 |
| HSV1-UL54-895 | + | CGACCCCGACCCCUGCG | 17 | 7573 |
| HSV1-UL54-896 | + | CGACCCCUGCGACGCCC | 17 | 7574 |
| HSV1-UL54-206 | + | GACCCCUGCGACGCCCA | 17 | 6969 |
| HSV1-UL54-898 | + | ACCCCUGCGACGCCCAC | 17 | 7575 |
| HSV1-UL54-899 | + | CUGCGACGCCCACGGCG | 17 | 7576 |
| HSV1-UL54-900 | + | UGCGACGCCCACGGCGU | 17 | 7577 |
| HSV1-UL54-901 | + | GCGACGCCCACGGCGUC | 17 | 7578 |
| HSV1-UL54-902 | + | ACGGCGUCCGCCGCGGG | 17 | 7579 |
| HSV1-UL54-208 | + | CGGCGUCCGCCGCGGGC | 17 | 6971 |
| HSV1-UL54-904 | + | CCGCGGGCAGGCUGGGC | 17 | 7580 |
| HSV1-UL54-905 | + | CGGGCAGGCUGGGCUUU | 17 | 7581 |
| HSV1-UL54-906 | + | CCUUGCCCCCGUGCUGC | 17 | 7582 |
| HSV1-UL54-60 | + | UGCCCCCGUGCUGCUCG | 17 | 6823 |
| HSV1-UL54-908 | + | CCGUGCUGCUCGGGGGA | 17 | 7583 |
| HSV1-UL54-909 | + | CGUGCUGCUCGGGGGAG | 17 | 7584 |

Table 4E provides exemplary targeting domains for knocking out the UL54 gene selected according to the second tier parameters. The targeting domains are selected based on location within the coding sequence, but downstream of the first 500 bp of the UL54 gene. It is contemplated herein that the targeting domain hybridizes to the target domain through complementary base pairing. Any of the targeting domains in the table can be used with a S. aureus Cas9 molecule that gives double stranded cleavage. Any of the targeting domains in the table can be used with a S. aureus Cas9 single-stranded break nucleases (nickases). In an embodiment, dual targeting is used to create two nicks on opposite DNA strands by using S. aureus Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 4E
2nd Tier

| | DNA | | Target | |
|---|---|---|---|---|
| gRNA Name | Strand | Targeting Domain | Site Length | Seq ID |
| HSV1-UL54-910 | − | CAGAACCAAUCGCAACCCUG | 20 | 7585 |
| HSV1-UL54-911 | − | CCACCCGGAGGCCAGCGGAC | 20 | 7586 |
| HSV1-UL54-912 | − | CAACCCUGGGGGACCCCGCC | 20 | 7587 |
| HSV1-UL54-254 | − | AACCCUGGGGGACCCCGCCC | 20 | 7017 |
| HSV1-UL54-914 | − | CUGGGGGACCCCGCCCCGGG | 20 | 7588 |
| HSV1-UL54-257 | − | UGGGGGACCCCGCCCCGGGG | 20 | 7020 |
| HSV1-UL54-916 | − | GGACCCCGCCCCGGGGCGGG | 20 | 7589 |
| HSV1-UL54-917 | − | CCCGCCCCGGGGCGGGGUGG | 20 | 7590 |
| HSV1-UL54-918 | − | CGGCCCCGGCGCCCCCCAUG | 20 | 7591 |
| HSV1-UL54-919 | − | UGGCGAGGCGUGGCGCGGCA | 20 | 7592 |
| HSV1-UL54-920 | − | CAGUGAGCAGCCCGACCCAC | 20 | 7593 |
| HSV1-UL54-268 | − | AGUGAGCAGCCCGACCCACC | 20 | 7031 |
| HSV1-UL54-922 | − | CCCGACCCACCCGGAGGCCA | 20 | 7594 |
| HSV1-UL54-271 | − | CACCCGGAGGCCAGCGGACA | 20 | 7034 |
| HSV1-UL54-924 | − | UGGCGAUUGCCCCCCCGCCC | 20 | 7595 |
| HSV1-UL54-925 | − | CGGACCCCCGCGCCCCGGCC | 20 | 7596 |
| HSV1-UL54-277 | − | GGACCCCCGCGCCCCGGCCC | 20 | 7040 |
| HSV1-UL54-927 | − | CCCCGCGCCCCGGCCCCGGA | 20 | 7597 |
| HSV1-UL54-928 | − | GUUGGUCCUGCGCUCCAUCU | 20 | 7598 |
| HSV1-UL54-929 | − | CGCGGCGGUCGACCGCAUCA | 20 | 7599 |
| HSV1-UL54-930 | − | CGGCGGUCGACCGCAUCAGC | 20 | 7600 |
| HSV1-UL54-931 | − | ACAGGUCAUGCACGACCCCU | 20 | 7601 |
| HSV1-UL54-285 | − | CAGGUCAUGCACGACCCCUU | 20 | 7048 |
| HSV1-UL54-286 | − | AGGUCAUGCACGACCCCUUU | 20 | 7049 |
| HSV1-UL54-287 | − | GGUCAUGCACGACCCCUUUG | 20 | 7050 |
| HSV1-UL54-935 | − | GGGGGCAGCCGUUUCCCGCC | 20 | 7602 |
| HSV1-UL54-936 | − | UUUCCCGCCGCGAAUAGCCC | 20 | 7603 |
| HSV1-UL54-937 | − | GCCCCUGGGCCCCGGUGCUG | 20 | 7604 |
| HSV1-UL54-938 | − | GGCCCCGGUGCUGGCGGGCC | 20 | 7605 |
| HSV1-UL54-296 | − | GCCCCGGUGCUGGCGGGCCA | 20 | 7059 |
| HSV1-UL54-940 | − | CCCGGUGCUGGCGGGCCAAG | 20 | 7606 |
| HSV1-UL54-941 | − | CCAAGGAGGGCCCUUUGACG | 20 | 7607 |
| HSV1-UL54-942 | − | CCCUUUGACGCCGAGACCAG | 20 | 7608 |
| HSV1-UL54-943 | − | GCCGAGACCAGACGGGUCUC | 20 | 7609 |
| HSV1-UL54-301 | − | CCGAGACCAGACGGGUCUCC | 20 | 7064 |
| HSV1-UL54-302 | − | CGAGACCAGACGGGUCUCCU | 20 | 7065 |
| HSV1-UL54-946 | − | AAACCUUGGUCGCCCACGGC | 20 | 7610 |
| HSV1-UL54-947 | − | CGCACUUUUGCCGGCAAUCC | 20 | 7611 |
| HSV1-UL54-948 | − | GCGCGACUGCGUGCUGCGCC | 20 | 7612 |
| HSV1-UL54-949 | − | GCUGCGCCAAGAAAAUUUCA | 20 | 7613 |
| HSV1-UL54-950 | − | CGAGGCGCUGGCCUCCGCCG | 20 | 7614 |
| HSV1-UL54-951 | − | ACAACCUGCCGCUGCGCCCC | 20 | 7615 |
| HSV1-UL54-952 | − | GCGCCCCCAGGACCCCAUUA | 20 | 7616 |
| HSV1-UL54-314 | − | CGCCCCCAGGACCCCAUUAU | 20 | 7077 |
| HSV1-UL54-954 | − | UCGGGACGACCGCGGCUGUG | 20 | 7617 |
| HSV1-UL54-955 | − | GGCCCUUUCUCCAGUGCUAC | 20 | 7618 |
| HSV1-UL54-956 | − | CUCCAGUGCUACCUGAAGGC | 20 | 7619 |
| HSV1-UL54-957 | − | AGGCGCGAGGCCUGUGCGGC | 20 | 7620 |
| HSV1-UL54-958 | − | GCGAGGCCUGUGCGGCCUGG | 20 | 7621 |
| HSV1-UL54-959 | − | UGUGUUCGCGGCGGCGUCUG | 20 | 7622 |
| HSV1-UL54-960 | − | GGCGGCGUCUGGCGGACAUU | 20 | 7623 |
| HSV1-UL54-961 | − | GGCCAGGCUCGCCAACCGCG | 20 | 7624 |
| HSV1-UL54-962 | − | ACCGCGUCGAGCGUGGCGUC | 20 | 7625 |
| HSV1-UL54-331 | − | CCGCGUCGAGCGUGGCGUCG | 20 | 7094 |
| HSV1-UL54-964 | − | CGACUACGCGACCCUUGGUG | 20 | 7626 |
| HSV1-UL54-333 | − | GACUACGCGACCCUUGGUGU | 20 | 7096 |
| HSV1-UL54-966 | − | CGCGACCCUUGGUGUCGGGG | 20 | 7627 |
| HSV1-UL54-336 | − | GCGACCCUUGGUGUCGGGGU | 20 | 7099 |
| HSV1-UL54-968 | − | GACCCUUGGUGUCGGGGUCG | 20 | 7628 |
| HSV1-UL54-969 | − | CCCUUGGUGUCGGGGUCGGA | 20 | 7629 |
| HSV1-UL54-970 | − | GAAGAUGCAUUUCUACCUCC | 20 | 7630 |
| HSV1-UL54-337 | − | AAGAUGCAUUUCUACCUCCC | 20 | 7100 |
| HSV1-UL54-972 | − | ACCUCCCCGGGGCCUGCAUG | 20 | 7631 |
| HSV1-UL54-973 | − | GGCCUGCAUGGCGGGCCUGA | 20 | 7632 |
| HSV1-UL54-974 | − | AAAUCCUAGACACGCACCGC | 20 | 7633 |
| HSV1-UL54-343 | − | AAUCCUAGACACGCACCGCC | 20 | 7106 |
| HSV1-UL54-976 | − | ACACGCACCGCCAGGAGUGU | 20 | 7634 |
| HSV1-UL54-977 | − | GGAGUGUUCGAGUCGUGUCU | 20 | 7635 |
| HSV1-UL54-978 | + | ACACUCCUGGCGGUGCGUGU | 20 | 7636 |
| HSV1-UL54-979 | + | GGGAGGUAGAAAUGCAUCUU | 20 | 7637 |
| HSV1-UL54-980 | + | ACACCAAGGGUCGCGUAGUC | 20 | 7638 |
| HSV1-UL54-353 | + | AGACACGACUCGAACACUCC | 20 | 7116 |
| HSV1-UL54-982 | + | GGCGGUGCGUGUCUAGGAUU | 20 | 7639 |
| HSV1-UL54-983 | + | GCGGUGCGUGUCUAGGAUUU | 20 | 7640 |
| HSV1-UL54-984 | + | UCAGGCCCGCCAUGCAGGCC | 20 | 7641 |
| HSV1-UL54-985 | + | CAUGCAGGCCCCGGGGAGGU | 20 | 7642 |
| HSV1-UL54-986 | + | GGAGGUAGAAAUGCAUCUUC | 20 | 7643 |
| HSV1-UL54-987 | + | UCCGACCCCGACACCAAGGG | 20 | 7644 |
| HSV1-UL54-988 | + | CGACCCCGACACCAAGGGUC | 20 | 7645 |
| HSV1-UL54-989 | + | ACCCCGACACCAAGGGUCGC | 20 | 7646 |
| HSV1-UL54-990 | + | CCCCGACACCAAGGGUCGCG | 20 | 7647 |
| HSV1-UL54-991 | + | CACCAAGGGUCGCGUAGUCG | 20 | 7648 |
| HSV1-UL54-992 | + | CGACGCCACGCUCGACGCGG | 20 | 7649 |
| HSV1-UL54-365 | + | GACGCCACGCUCGACGCGGU | 20 | 7128 |
| HSV1-UL54-994 | + | CGCGGUUGGCGAGCCUGGCC | 20 | 7650 |
| HSV1-UL54-995 | + | AAUGUCCGCCAGACGCCGCC | 20 | 7651 |
| HSV1-UL54-996 | + | CAGACGCCGCCGCGAACACA | 20 | 7652 |
| HSV1-UL54-997 | + | CCAGGCCGCACAGGCCUCGC | 20 | 7653 |
| HSV1-UL54-998 | + | GCCGCACAGGCCUCGCGCCU | 20 | 7654 |
| HSV1-UL54-999 | + | GCCUUCAGGUAGCACUGGAG | 20 | 7655 |
| HSV1-UL54-1000 | + | GUAGCACUGGAGAAAGGGCC | 20 | 7656 |
| HSV1-UL54-1001 | + | CACAGCCGCGGUCGUCCCGA | 20 | 7657 |
| HSV1-UL54-1002 | + | AUAAUGGGGUCCUGGGGGCG | 20 | 7658 |
| HSV1-UL54-1003 | + | UAAUGGGGUCCUGGGGGCGC | 20 | 7659 |
| HSV1-UL54-1004 | + | GGGGCGCAGCGGCAGGUUGU | 20 | 7660 |
| HSV1-UL54-1005 | + | CGGCGGAGGCCAGCGCCUCG | 20 | 7661 |
| HSV1-UL54-1006 | + | UGAAAUUUUCUUGGCGCAGC | 20 | 7662 |
| HSV1-UL54-1007 | + | GGCGCAGCACGCAGUCGCGC | 20 | 7663 |
| HSV1-UL54-1008 | + | GGAUUGCCGGCAAAAGUGCG | 20 | 7664 |
| HSV1-UL54-1009 | + | GCCGUGGGCGACCAAGGUUU | 20 | 7665 |
| HSV1-UL54-1010 | + | AGGAGACCCGUCUGGUCUCG | 20 | 7666 |
| HSV1-UL54-1011 | + | GGAGACCCGUCUGGUCUCGG | 20 | 7667 |
| HSV1-UL54-1012 | + | GAGACCCGUCUGGUCUCGGC | 20 | 7668 |
| HSV1-UL54-1013 | + | CUGGUCUCGGCGUCAAAGGG | 20 | 7669 |
| HSV1-UL54-1014 | + | CGUCAAAGGGCCCUCCUUGG | 20 | 7670 |
| HSV1-UL54-1015 | + | CUUGGCCCGCCAGCACCGGG | 20 | 7671 |
| HSV1-UL54-1016 | + | UGGCCCGCCAGCACCGGGGC | 20 | 7672 |
| HSV1-UL54-1017 | + | GGCCCGCCAGCACCGGGGCC | 20 | 7673 |
| HSV1-UL54-1018 | + | CAGCACCGGGGCCCAGGGGC | 20 | 7674 |
| HSV1-UL54-420 | + | GGGCUAUUCGCGGCGGGAAA | 20 | 7183 |
| HSV1-UL54-1020 | + | GGCGGGAAACGGCUGCCCCC | 20 | 7675 |
| HSV1-UL54-1021 | + | CAAAGGGGUCGUGCAUGACC | 20 | 7676 |
| HSV1-UL54-1022 | + | AAAGGGGUCGUGCAUGACCU | 20 | 7677 |
| HSV1-UL54-1023 | + | AAGGGGUCGUGCAUGACCUG | 20 | 7678 |
| HSV1-UL54-1024 | + | AGGGGUCGUGCAUGACCUGU | 20 | 7679 |
| HSV1-UL54-1025 | + | GCUGAUGCGGUCGACCGCCG | 20 | 7680 |
| HSV1-UL54-1026 | + | UGAUGCGGUCGACCGCCGCG | 20 | 7681 |
| HSV1-UL54-1027 | + | AGAUGGAGCGCAGGACCAAC | 20 | 7682 |
| HSV1-UL54-1028 | + | UCCGGGGCCGGGGCGCGGGG | 20 | 7683 |
| HSV1-UL54-1029 | + | GGGCCGGGGCGCGGGGGUCC | 20 | 7684 |
| HSV1-UL54-446 | + | GGCCGGGGCGCGGGGGUCCG | 20 | 7209 |
| HSV1-UL54-1031 | + | GGGCGGGGGGGCAAUCGCCA | 20 | 7685 |
| HSV1-UL54-1032 | + | UGUCCGCUGGCCUCCGGGUG | 20 | 7686 |
| HSV1-UL54-1033 | + | GUCCGCUGGCCUCCGGGUGG | 20 | 7687 |
| HSV1-UL54-1034 | + | UGGCCUCCGGGUGGGUCGGG | 20 | 7688 |
| HSV1-UL54-1035 | + | GGUGGGUCGGGCUGCUCACU | 20 | 7689 |
| HSV1-UL54-1036 | + | GUGGGUCGGGCUGCUCACUG | 20 | 7690 |
| HSV1-UL54-469 | + | UGCCGCGCCACGCCUCGCCA | 20 | 7232 |
| HSV1-UL54-1038 | + | CAUGGGGGGCGCCGGGGCCG | 20 | 7691 |
| HSV1-UL54-1039 | + | CCACCCCGCCCCGGGGCGGG | 20 | 7692 |
| HSV1-UL54-1040 | + | CCCGCCCCGGGGCGGGGUCC | 20 | 7693 |
| HSV1-UL54-484 | + | CCCCGGGGCGGGGUCCCCCA | 20 | 7247 |
| HSV1-UL54-1042 | + | CCCGGGGCGGGGUCCCCCAG | 20 | 7694 |
| HSV1-UL54-1043 | + | GGGCGGGGUCCCCCAGGGUU | 20 | 7695 |
| HSV1-UL54-1044 | + | GGCGGGGUCCCCCAGGGUUG | 20 | 7696 |
| HSV1-UL54-1045 | + | CAGGGUUGCGAUUGGUUCUG | 20 | 7697 |
| HSV1-UL54-1045 | + | CAGGGUUGCGAUUGGUUCUG | 20 | 7697 |
| HSV1-UL54-1045 | + | CAGGGUUGCGAUUGGUUCUG | 20 | 7697 |
| HSV1-UL54-1045 | + | CAGGGUUGCGAUUGGUUCUG | 20 | 7697 |
| HSV1-UL54-488 | − | AACCAAUCGCAACCCUG | 17 | 7251 |
| HSV1-UL54-1050 | − | CCCGGAGGCCAGCGGAC | 17 | 7698 |
| HSV1-UL54-1051 | − | CCCUGGGGGACCCCGCC | 17 | 7699 |
| HSV1-UL54-490 | − | CCUGGGGGACCCCGCCC | 17 | 7253 |
| HSV1-UL54-1053 | − | GGGGACCCCGCCCCGGG | 17 | 7700 |
| HSV1-UL54-493 | − | GGGACCCCGCCCCGGGG | 17 | 7256 |
| HSV1-UL54-1055 | − | CCCCGCCCCGGGGCGGG | 17 | 7701 |
| HSV1-UL54-1056 | − | GCCCCGGGGCGGGGUGG | 17 | 7702 |
| HSV1-UL54-1057 | − | CCCCGGCGCCCCCCAUG | 17 | 7703 |
| HSV1-UL54-1058 | − | CGAGGCGUGGCGCGGCA | 17 | 7704 |
| HSV1-UL54-1059 | − | UGAGCAGCCCGACCCAC | 17 | 7705 |
| HSV1-UL54-504 | − | GAGCAGCCCGACCCACC | 17 | 7267 |
| HSV1-UL54-1061 | − | GACCCACCCGGAGGCCA | 17 | 7706 |
| HSV1-UL54-507 | − | CCGGAGGCCAGCGGACA | 17 | 7270 |
| HSV1-UL54-1063 | − | CGAUUGCCCCCCCGCCC | 17 | 7707 |
| HSV1-UL54-1064 | − | ACCCCCGCGCCCCGGCC | 17 | 7708 |
| HSV1-UL54-513 | − | CCCCCGCGCCCCGGCCC | 17 | 7276 |
| HSV1-UL54-1066 | − | CGCGCCCCGGCCCCGGA | 17 | 7709 |
| HSV1-UL54-1067 | − | GGUCCUGCGCUCCAUCU | 17 | 7710 |
| HSV1-UL54-1068 | − | GGCGGUCGACCGCAUCA | 17 | 7711 |
| HSV1-UL54-1069 | − | CGGUCGACCGCAUCAGC | 17 | 7712 |
| HSV1-UL54-1070 | − | GGUCAUGCACGACCCCU | 17 | 7713 |
| HSV1-UL54-521 | − | GUCAUGCACGACCCCUU | 17 | 7284 |
| HSV1-UL54-522 | − | UCAUGCACGACCCCUUU | 17 | 7285 |
| HSV1-UL54-523 | − | CAUGCACGACCCCUUUG | 17 | 7286 |
| HSV1-UL54-1074 | − | GGCAGCCGUUUCCCGCC | 17 | 7714 |
| HSV1-UL54-1075 | − | CCCGCCGCGAAUAGCCC | 17 | 7715 |
| HSV1-UL54-1076 | − | CCUGGGCCCCGGUGCUG | 17 | 7716 |
| HSV1-UL54-1077 | − | CCCGGUGCUGGCGGGCC | 17 | 7717 |
| HSV1-UL54-532 | − | CCGGUGCUGGCGGGCCA | 17 | 7295 |
| HSV1-UL54-1079 | − | GGUGCUGGCGGGCCAAG | 17 | 7718 |
| HSV1-UL54-1080 | − | AGGAGGGCCCUUUGACG | 17 | 7719 |
| HSV1-UL54-1081 | − | UUUGACGCCGAGACCAG | 17 | 7720 |
| HSV1-UL54-1082 | − | GAGACCAGACGGGUCUC | 17 | 7721 |
| HSV1-UL54-537 | − | AGACCAGACGGGUCUCC | 17 | 7300 |
| HSV1-UL54-538 | − | GACCAGACGGGUCUCCU | 17 | 7301 |
| HSV1-UL54-1085 | − | CCUUGGUCGCCCACGGC | 17 | 7722 |
| HSV1-UL54-1086 | − | ACUUUUGCCGGCAAUCC | 17 | 7723 |
| HSV1-UL54-1087 | − | CGACUGCGUGCUGCGCC | 17 | 7724 |
| HSV1-UL54-1088 | − | GCGCCAAGAAAAUUUCA | 17 | 7725 |
| HSV1-UL54-1089 | − | GGCGCUGGCCUCCGCCG | 17 | 7726 |
| HSV1-UL54-1090 | − | ACCUGCCGCUGCGCCCC | 17 | 7727 |
| HSV1-UL54-1091 | − | CCCCCAGGACCCCAUUA | 17 | 7728 |
| HSV1-UL54-550 | − | CCCCAGGACCCCAUUAU | 17 | 7313 |
| HSV1-UL54-1093 | − | GGACGACCGCGGCUGUG | 17 | 7729 |
| HSV1-UL54-1094 | − | CCUUUCUCCAGUGCUAC | 17 | 7730 |
| HSV1-UL54-1095 | − | CAGUGCUACCUGAAGGC | 17 | 7731 |
| HSV1-UL54-1096 | − | CGCGAGGCCUGUGCGGC | 17 | 7732 |
| HSV1-UL54-1097 | − | AGGCCUGUGCGGCCUGG | 17 | 7733 |
| HSV1-UL54-1098 | − | GUUCGCGGCGGCGUCUG | 17 | 7734 |
| HSV1-UL54-1099 | − | GGCGUCUGGCGGACAUU | 17 | 7735 |
| HSV1-UL54-1100 | − | CAGGCUCGCCAACCGCG | 17 | 7736 |
| HSV1-UL54-1101 | − | GCGUCGAGCGUGGCGUC | 17 | 7737 |
| HSV1-UL54-567 | − | CGUCGAGCGUGGCGUCG | 17 | 7330 |
| HSV1-UL54-1103 | − | CUACGCGACCCUUGGUG | 17 | 7738 |
| HSV1-UL54-569 | − | UACGCGACCCUUGGUGU | 17 | 7332 |
| HSV1-UL54-1105 | − | GACCCUUGGUGUCGGGG | 17 | 7739 |
| HSV1-UL54-572 | − | ACCCUUGGUGUCGGGGU | 17 | 7335 |
| HSV1-UL54-1107 | − | CCUUGGUGUCGGGGUCG | 17 | 7740 |
| HSV1-UL54-1108 | − | UUGGUGUCGGGGUCGGA | 17 | 7741 |
| HSV1-UL54-1109 | − | GAUGCAUUUCUACCUCC | 17 | 7742 |
| HSV1-UL54-573 | − | AUGCAUUUCUACCUCCC | 17 | 7336 |
| HSV1-UL54-1111 | − | UCCCCGGGGCCUGCAUG | 17 | 7743 |
| HSV1-UL54-1112 | − | CUGCAUGGCGGGCCUGA | 17 | 7744 |
| HSV1-UL54-1113 | − | UCCUAGACACGCACCGC | 17 | 7745 |
| HSV1-UL54-579 | − | CCUAGACACGCACCGCC | 17 | 7342 |
| HSV1-UL54-1115 | − | CGCACCGCCAGGAGUGU | 17 | 7746 |
| HSV1-UL54-1116 | − | GUGUUCGAGUCGUGUCU | 17 | 7747 |
| HSV1-UL54-1117 | − | ACACUCCUGGCGGUGCG | 17 | 7748 |
| HSV1-UL54-1118 | − | GGGAGGUAGAAAUGCAU | 17 | 7749 |
| HSV1-UL54-1119 | − | ACACCAAGGGUCGCGUA | 17 | 7750 |
| HSV1-UL54-1120 | − | AGACACGACUCGAACAC | 17 | 7751 |
| HSV1-UL54-1121 | − | GGCGGUGCGUGUCUAGG | 17 | 7752 |
| HSV1-UL54-1122 | − | GCGGUGCGUGUCUAGGA | 17 | 7753 |
| HSV1-UL54-1123 | − | UCAGGCCCGCCAUGCAG | 17 | 7754 |
| HSV1-UL54-1124 | − | CAUGCAGGCCCCGGGGA | 17 | 7755 |
| HSV1-UL54-1125 | − | GGAGGUAGAAAUGCAUC | 17 | 7756 |
| HSV1-UL54-599 | − | UCCGACCCCGACACCAA | 17 | 7362 |
| HSV1-UL54-1127 | − | CGACCCCGACACCAAGG | 17 | 7757 |
| HSV1-UL54-1128 | − | ACCCCGACACCAAGGGU | 17 | 7758 |
| HSV1-UL54-1129 | − | CCCCGACACCAAGGGUC | 17 | 7759 |
| HSV1-UL54-1130 | − | CACCAAGGGUCGCGUAG | 17 | 7760 |
| HSV1-UL54-600 | − | CGACGCCACGCUCGACG | 17 | 7363 |
| HSV1-UL54-1132 | − | GACGCCACGCUCGACGC | 17 | 7761 |
| HSV1-UL54-1133 | − | CGCGGUUGGCGAGCCUG | 17 | 7762 |
| HSV1-UL54-1134 | − | AAUGUCCGCCAGACGCC | 17 | 7763 |
| HSV1-UL54-1135 | − | CAGACGCCGCCGCGAAC | 17 | 7764 |
| HSV1-UL54-1136 | − | CCAGGCCGCACAGGCCU | 17 | 7765 |
| HSV1-UL54-1137 | − | GCCGCACAGGCCUCGCG | 17 | 7766 |
| HSV1-UL54-1138 | − | GCCUUCAGGUAGCACUG | 17 | 7767 |
| HSV1-UL54-1139 | − | GUAGCACUGGAGAAAGG | 17 | 7768 |
| HSV1-UL54-1140 | − | CACAGCCGCGGUCGUCC | 17 | 7769 |
| HSV1-UL54-1141 | − | AUAAUGGGGUCCUGGGG | 17 | 7770 |
| HSV1-UL54-1142 | − | UAAUGGGGUCCUGGGGG | 17 | 7771 |
| HSV1-UL54-1143 | − | GGGGCGCAGCGGCAGGU | 17 | 7772 |
| HSV1-UL54-1144 | − | CGGCGGAGGCCAGCGCC | 17 | 7773 |
| HSV1-UL54-1145 | − | UGAAAUUUUCUUGGCGC | 17 | 7774 |
| HSV1-UL54-1146 | − | GGCGCAGCACGCAGUCG | 17 | 7775 |
| HSV1-UL54-1147 | − | GGAUUGCCGGCAAAAGU | 17 | 7776 |
| HSV1-UL54-1148 | − | GCCGUGGGCGACCAAGG | 17 | 7777 |
| HSV1-UL54-1149 | − | AGGAGACCCGUCUGGUC | 17 | 7778 |
| HSV1-UL54-643 | − | GGAGACCCGUCUGGUCU | 17 | 7406 |
| HSV1-UL54-1151 | − | GAGACCCGUCUGGUCUC | 17 | 7779 |
| HSV1-UL54-645 | − | CUGGUCUCGGCGUCAAA | 17 | 7408 |
| HSV1-UL54-646 | − | CGUCAAAGGGCCCUCCU | 17 | 7409 |
| HSV1-UL54-648 | − | CUUGGCCCGCCAGCACC | 17 | 7411 |
| HSV1-UL54-1155 | − | UGGCCCGCCAGCACCGG | 17 | 7780 |
| HSV1-UL54-1156 | − | GGCCCGCCAGCACCGGG | 17 | 7781 |
| HSV1-UL54-1157 | − | CAGCACCGGGGCCCAGG | 17 | 7782 |
| HSV1-UL54-1158 | − | GGGCUAUUCGCGGCGGG | 17 | 7783 |
| HSV1-UL54-1159 | − | GGCGGGAAACGGCUGCC | 17 | 7784 |
| HSV1-UL54-1160 | − | CAAAGGGGUCGUGCAUG | 17 | 7785 |
| HSV1-UL54-1161 | − | AAAGGGGUCGUGCAUGA | 17 | 7786 |
| HSV1-UL54-1162 | − | AAGGGGUCGUGCAUGAC | 17 | 7787 |
| HSV1-UL54-1163 | − | AGGGGUCGUGCAUGACC | 17 | 7788 |
| HSV1-UL54-1164 | − | GCUGAUGCGGUCGACCG | 17 | 7789 |
| HSV1-UL54-1165 | − | UGAUGCGGUCGACCGCC | 17 | 7790 |
| HSV1-UL54-1166 | − | AGAUGGAGCGCAGGACC | 17 | 7791 |
| HSV1-UL54-680 | − | UCCGGGGCCGGGGCGCG | 17 | 7443 |
| HSV1-UL54-1168 | − | GGGCCGGGGCGCGGGGG | 17 | 7792 |
| HSV1-UL54-1169 | − | GGCCGGGGCGCGGGGGU | 17 | 7793 |
| HSV1-UL54-1170 | − | GGGCGGGGGGGCAAUCG | 17 | 7794 |
| HSV1-UL54-1171 | − | UGUCCGCUGGCCUCCGG | 17 | 7795 |
| HSV1-UL54-701 | − | GUCCGCUGGCCUCCGGG | 17 | 7464 |
| HSV1-UL54-704 | − | UGGCCUCCGGGUGGGUC | 17 | 7467 |
| HSV1-UL54-1174 | − | GGUGGGUCGGGCUGCUC | 17 | 7796 |
| HSV1-UL54-1175 | − | GUGGGUCGGGCUGCUCA | 17 | 7797 |
| HSV1-UL54-1176 | − | UGCCGCGCCACGCCUCG | 17 | 7798 |
| HSV1-UL54-1177 | − | CAUGGGGGGCGCCGGGG | 17 | 7799 |
| HSV1-UL54-717 | − | CCACCCCGCCCCGGGGC | 17 | 7480 |
| HSV1-UL54-496 | − | CCCGCCCCGGGGCGGGG | 17 | 7259 |
| HSV1-UL54-1180 | − | CCCCGGGGCGGGGUCCC | 17 | 7800 |
| HSV1-UL54-1181 | − | CCCGGGGCGGGGUCCCC | 17 | 7801 |
| HSV1-UL54-1182 | − | GGGCGGGGUCCCCCAGG | 17 | 7802 |
| HSV1-UL54-1183 | − | GGCGGGGUCCCCCAGGG | 17 | 7803 |
| HSV1-UL54-1184 | − | CAGGGUUGCGAUUGGUU | 17 | 7804 |
| HSV1-UL54-1185 | − | AGGGUUGCGAUUGGUUC | 17 | 7805 |
| HSV1-UL54-1186 | − | GGGUUGCGAUUGGUUCU | 17 | 7806 |
| HSV1-UL54-1187 | − | GGUUGCGAUUGGUUCUG | 17 | 7807 |

Table 4F provides exemplary targeting domains for knocking out the UL54 gene selected according to the first tier parameters. The targeting domains are selected based on location within first 500 bp of the coding sequence of the UL54 gene. It is contemplated herein that the targeting domain hybridizes to the target domain through complementary base pairing. Any of the targeting domains in the table can be used with a N. meningitidis Cas9 molecule that gives double stranded cleavage. Any of the targeting domains in the table can be used with a N. meningitidis Cas9 single-stranded break nucleases (nickases). In an embodiment, dual targeting is used to create two nicks on opposite DNA strands by using N. meningitidis Cas9 nickases with two targeting domains that are complementary to opposite DNA strands, e.g., a gRNA comprising any minus strand targeting domain may be paired any gRNA comprising a plus strand targeting domain provided that the two gRNAs are oriented on the DNA such that PAMs face outward and the distance between the 5′ ends of the gRNAs is 0-50 bp.

TABLE 4F
1st Tier

| | | | Target | |
|---|---|---|---|---|
| | DNA | | Site | |
| gRNA Name | Strand | Targeting Domain | Length | Seq ID |
| HSV1-UL54-274 | − | ACCCCCCCCGCUAAUGA | 20 | 7037 |
| | | CGC | | |
| HSV1-UL54-723 | + | GCGUCAUUAGCGGGGGG | 20 | 7485 |
| | | GGU | | |
| HSV1-UL54-510 | − | CCCCCCGCUAAUGACGC | 17 | 7273 |
| HSV1-UL54-725 | + | GCGUCAUUAGCGGGGGG | 17 | 7486 |

Table 5A provides exemplary targeting domains for knocking out the ULS9 gene selected according to the first tier parameters. The targeting domains bind within the first 500 bp of the coding sequence (e.g., within 500 bp downstream from the start codon), have a high level of orthogonality, and start with a 5′G. It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 5A
1st Tier

| | DNA | | Target | Seq ID |
|---|---|---|---|---|
| gRNA Name | Strand | Targeting Domain | Site Length | NO: |
| HSV1-UL19-179 | + | GGACCCGGUCGGCACCA | 17 | 569 |
| HSV1-UL19-136 | + | GAUACCCGUCCCGUCCA | 17 | 540 |
| HSV1-UL19-168 | + | GCAAUACGACCCCAGCA | 17 | 448 |
| HSV1-UL19-3209 | + | GCGAGCGAGCUGUUGGA | 17 | 7808 |
| HSV1-UL19-134 | + | GUUGGAUGGCGCGCAAC | 17 | 422 |
| HSV1-UL19-165 | + | GAAAGCGCACGAGCGAC | 17 | 445 |
| HSV1-UL19-97 | − | GCGACCCUCCGGGAUAC | 17 | 390 |
| HSV1-UL19-121 | − | GAACGCCGCCUUCAGCC | 17 | 529 |
| HSV1-UL19-182 | + | GCGGCAUACCGGUAUCC | 17 | 571 |
| HSV1-UL19-3210 | − | GCUCGCUCGCAACGUCC | 17 | 7809 |
| HSV1-UL19-156 | + | GUUGCUCGAUGGGGUGC | 17 | 558 |
| HSV1-UL19-107 | − | GUCGCUCGUGCGCUUUC | 17 | 400 |
| HSV1-UL19-98 | − | GGGAUACCGGUAUGCCG | 17 | 391 |
| HSV1-UL19-117 | − | GGGGCGCGUACAGUUCG | 17 | 413 |
| HSV1-UL19-105 | − | GAGUUCGACGCCCUGCU | 17 | 398 |
| HSV1-UL19-85 | + | GAGGGACCCGGUCGGCACCA | 20 | 512 |
| HSV1-UL19-3211 | + | GUCGCGGUUGGGAGCGGCCA | 20 | 7810 |
| HSV1-UL19-42 | + | GCCGAUACCCGUCCCGUCCA | 20 | 483 |
| HSV1-UL19-74 | + | GUUGCAAUACGACCCCAGCA | 20 | 440 |
| HSV1-UL19-3212 | + | GUUGCGAGCGAGCUGUUGGA | 20 | 7811 |
| HSV1-UL19-40 | + | GCUGUUGGAUGGCGCGCAAC | 20 | 421 |
| HSV1-UL19-87 | + | GCACCAUGGCCGCGGCAUAC | 20 | 453 |
| HSV1-UL19-26 | − | GACCAAGAUUAUCGACCGCC | 20 | 412 |
| HSV1-UL19-48 | + | GGGCGAUGGCCUCGGUGGCC | 20 | 489 |
| HSV1-UL19-88 | + | GCCGCGGCAUACCGGUAUCC | 20 | 514 |
| HSV1-UL19-78 | + | GUUUGCAUCGGAGCGCACGC | 20 | 506 |
| HSV1-UL19-190 | − | GCAACGUCCAGGCCGUCCUC | 20 | 576 |
| HSV1-UL19-18 | − | GUGCGUGUGUACCAAGUUUC | 20 | 404 |
| HSV1-UL19-65 | + | GGGCCGUCGCGGGCAAUCAG | 20 | 430 |
| HSV1-UL19-8 | − | GUCCCUCCUUAGCACGAUCG | 20 | 465 |
| HSV1-UL19-32 | − | GGCCAUCGCCCUGCUCACGG | 20 | 476 |
| HSV1-UL19-89 | + | GCGGCAUACCGGUAUCCCGG | 20 | 515 |
| HSV1-UL19-61 | + | GUGGGUUGCUCGAUGGGGUG | 20 | 500 |
| HSV1-UL19-76 | + | GUCGUACAGGCUGUUUGCAU | 20 | 442 |
| HSV1-UL19-39 | − | GCCCUGGACGGGACGGGUAU | 20 | 482 |
| HSV1-UL19-70 | + | GUAGGCCAGCUCCGGAAACU | 20 | 436 |
| HSV1-UL19-11 | − | GUCGAGUUCGACGCCCUGCU | 20 | 393 |
| HSV1-UL19-55 | + | GGCCCGGCGGUCGAUAAUCU | 20 | 496 |
| HSV1-UL19-3213 | + | GGACGUUGCGAGCGAGCUGU | 20 | 7812 |

Table 5B provides exemplary targeting domains for knocking out the UL19 gene selected according to the second tier parameters. The targeting domains bind within the first 500 bp of the coding sequence (e.g., within 500 bp downstream from the start codon) and have a high level of orthogonality. It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 5B
2nd Tier

| | DNA | | Target | Seq ID |
|---|---|---|---|---|
| gRNA Name | Strand | Targeting Domain | Site Length | NO: |
| HSV1-UL19-166 | + | AAAGCGCACGAGCGACA | 17 | 446 |
| HSV1-UL19-3214 | − | AUCUCCACGCGACCCCA | 17 | 7813 |
| HSV1-UL19-99 | − | CCGGUAUGCCGCGGCCA | 17 | 392 |
| HSV1-UL19-115 | − | CUGGCCUACAUGAACGA | 17 | 527 |
| HSV1-UL19-118 | − | CCGCUGAUUGCCCGCGA | 17 | 414 |
| HSV1-UL19-176 | + | CUCGAUCGUGCUAAGGA | 17 | 452 |
| HSV1-UL19-184 | + | CAUACCGGUAUCCCGGA | 17 | 573 |
| HSV1-UL19-174 | + | CCACCUCGAUCGUGCUA | 17 | 565 |
| HSV1-UL19-181 | + | CCAUGGCCGCGGCAUAC | 17 | 454 |
| HSV1-UL19-169 | + | CGAACUCGACGUCGUAC | 17 | 449 |
| HSV1-UL19-1442 | + | ACGCCCCGAGGACGGCC | 17 | 1828 |
| HSV1-UL19-135 | + | CGAUACCCGUCCCGUCC | 17 | 424 |
| HSV1-UL19-172 | + | UGCAUCGGAGCGCACGC | 17 | 563 |
| HSV1-UL19-119 | − | CCAAGAUUAUCGACCGC | 17 | 415 |
| HSV1-UL19-160 | + | CGCGGGCAAUCAGCGGC | 17 | 560 |
| HSV1-UL19-104 | − | CGAGUUCGACGCCCUGC | 17 | 524 |
| HSV1-UL19-171 | + | UUGCAUCGGAGCGCACG | 17 | 562 |
| HSV1-UL19-125 | − | CCAUCGCCCUGCUCACG | 17 | 418 |
| HSV1-UL19-102 | − | CCUCCUUAGCACGAUCG | 17 | 522 |
| HSV1-UL19-175 | + | CCUCGAUCGUGCUAAGG | 17 | 566 |
| HSV1-UL19-103 | − | CCUUAGCACGAUCGAGG | 17 | 523 |
| HSV1-UL19-126 | − | CAUCGCCCUGCUCACGG | 17 | 533 |
| HSV1-UL19-106 | − | AGUUCGACGCCCUGCUG | 17 | 399 |
| HSV1-UL19-153 | + | UGUGUGGGUUGCUCGAU | 17 | 428 |
| HSV1-UL19-133 | − | CUGGACGGGACGGGUAU | 17 | 539 |
| HSV1-UL19-582 | − | AACGUCCAGGCCGUCCU | 17 | 968 |
| HSV1-UL19-149 | + | CCGGCGGUCGAUAAUCU | 17 | 553 |
| HSV1-UL19-3215 | + | CGUUGCGAGCGAGCUGU | 17 | 7814 |
| HSV1-UL19-187 | + | CCCGGAGGGUCGCGGUU | 17 | 460 |
| HSV1-UL19-72 | + | CAGAAAGCGCACGAGCGACA | 20 | 438 |
| HSV1-UL19-3216 | − | CCGAUCUCCACGCGACCCCA | 20 | 7815 |
| HSV1-UL19-5 | − | AUACCGGUAUGCCGCGGCCA | 20 | 389 |
| HSV1-UL19-29 | − | CGAGGCCAUCGCCCUGCUCA | 20 | 474 |
| HSV1-UL19-52 | + | CAGGCUGAAGGCGGCGUUCA | 20 | 493 |
| HSV1-UL19-24 | − | CAGCCGCUGAUUGCCCGCGA | 20 | 410 |
| HSV1-UL19-45 | + | CUCCCCCGUGAGCAGGGCGA | 20 | 486 |
| HSV1-UL19-82 | + | CACCUCGAUCGUGCUAAGGA | 20 | 451 |
| HSV1-UL19-90 | + | CGGCAUACCGGUAUCCCGGA | 20 | 516 |
| HSV1-UL19-80 | + | ACGCCACCUCGAUCGUGCUA | 20 | 508 |
| HSV1-UL19-71 | + | CCAGAAAGCGCACGAGCGAC | 20 | 437 |
| HSV1-UL19-3 | − | ACCGCGACCCUCCGGGAUAC | 20 | 387 |
| HSV1-UL19-75 | + | CGUCGAACUCGACGUCGUAC | 20 | 441 |
| HSV1-UL19-7 | − | CCGCGGCCAUGGUGCCGACC | 20 | 464 |
| HSV1-UL19-975 | + | CAAACGCCCCGAGGACGGCC | 20 | 1361 |
| HSV1-UL19-53 | + | UGAAGGCGGCGUUCAGGGCC | 20 | 494 |
| HSV1-UL19-3217 | − | ACAGCUCGCUCGCAACGUCC | 20 | 7816 |
| HSV1-UL19-41 | + | CGCCGAUACCCGUCCCGUCC | 20 | 423 |
| HSV1-UL19-73 | + | UGUUGCAAUACGACCCCAGC | 20 | 439 |
| HSV1-UL19-25 | − | UGACCAAGAUUAUCGACCGC | 20 | 411 |
| HSV1-UL19-66 | + | CGUCGCGGGCAAUCAGCGGC | 20 | 503 |
| HSV1-UL19-10 | − | CGUCGAGUUCGACGCCCUGC | 20 | 467 |
| HSV1-UL19-62 | + | UGGGUUGCUCGAUGGGGUGC | 20 | 501 |
| HSV1-UL19-1 | − | CGCUCCCAACCGCGACCCUC | 20 | 461 |
| HSV1-UL19-69 | + | UCGUUCAUGUAGGCCAGCUC | 20 | 435 |
| HSV1-UL19-15 | − | UCGUGCGCUUUCUGGAGCUC | 20 | 397 |
| HSV1-UL19-51 | + | CCAGGCUGAAGGCGGCGUUC | 20 | 492 |
| HSV1-UL19-13 | − | CCUGUCGCUCGUGCGCUUUC | 20 | 395 |
| HSV1-UL19-77 | + | UGUUUGCAUCGGAGCGCACG | 20 | 505 |
| HSV1-UL19-31 | − | AGGCCAUCGCCCUGCUCACG | 20 | 417 |
| HSV1-UL19-86 | + | CCCGGUCGGCACCAUGGCCG | 20 | 513 |
| HSV1-UL19-4 | − | UCCGGGAUACCGGUAUGCCG | 20 | 388 |
| HSV1-UL19-191 | − | CAACGUCCAGGCCGUCCUCG | 20 | 577 |
| HSV1-UL19-63 | + | CGAUGGGGUGCGGGCCGUCG | 20 | 502 |
| HSV1-UL19-91 | + | ACCGGUAUCCCGGAGGGUCG | 20 | 455 |
| HSV1-UL19-23 | − | CGAGGGGCGCGUACAGUUCG | 20 | 409 |
| HSV1-UL19-81 | + | CCACCUCGAUCGUGCUAAGG | 20 | 509 |
| HSV1-UL19-9 | − | CCUCCUUAGCACGAUCGAGG | 20 | 466 |
| HSV1-UL19-54 | + | AGGCGGCGUUCAGGGCCCGG | 20 | 495 |
| HSV1-UL19-17 | − | UCUGGAGCUCGGGUUGUCGG | 20 | 403 |
| HSV1-UL19-33 | − | CAUCGCCCUGCUCACGGGGG | 20 | 419 |
| HSV1-UL19-67 | + | CGCGGGCAAUCAGCGGCUGG | 20 | 433 |
| HSV1-UL19-3218 | + | CGCGGUUGGGAGCGGCCAUG | 20 | 7817 |
| HSV1-UL19-60 | + | UGUGUGUGGGUUGCUCGAUG | 20 | 499 |
| HSV1-UL19-12 | − | UCGAGUUCGACGCCCUGCUG | 20 | 394 |
| HSV1-UL19-3219 | + | UCGCGGUUGGGAGCGGCCAU | 20 | 7818 |
| HSV1-UL19-59 | + | UUGUGUGUGGGUUGCUCGAU | 20 | 426 |
| HSV1-UL19-189 | − | CGCAACGUCCAGGCCGUCCU | 20 | 575 |
| HSV1-UL19-14 | − | CUCGUGCGCUUUCUGGAGCU | 20 | 396 |
| HSV1-UL19-68 | + | UACGCGCCCCUCGUUCAUGU | 20 | 504 |
| HSV1-UL19-16 | − | CUUUCUGGAGCUCGGGUUGU | 20 | 468 |
| HSV1-UL19-93 | + | UAUCCCGGAGGGUCGCGGUU | 20 | 457 |

Table 5C provides exemplary targeting domains for knocking out the ULS9 gene selected according to the third tier parameters. The targeting domains bind within the first 500 bp of the coding sequence (e.g., within 500 bp downstream from the start codon) and start with a 5′G. It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 5C
3rd Tier

| | DNA | | Target | Seq ID |
|---|---|---|---|---|
| gRNA Name | Strand | Targeting Domain | Site Length | NO: |
| HSV1-UL19-3220 | + | GCGGUUGGGAGCGGCCA | 17 | 7819 |
| HSV1-UL19-138 | + | GGCCUCCCCCGUGAGCA | 17 | 542 |
| HSV1-UL19-123 | − | GGCCAUCGCCCUGCUCA | 17 | 531 |
| HSV1-UL19-146 | + | GCUGAAGGCGGCGUUCA | 17 | 550 |
| HSV1-UL19-152 | + | GUGUGUGGGUUGCUCGA | 17 | 427 |
| HSV1-UL19-131 | − | GGAGGCCCUGGACGGGA | 17 | 537 |
| HSV1-UL19-173 | + | GGGAAAAAAAAUCAAAC | 17 | 564 |
| HSV1-UL19-124 | − | GCCAUCGCCCUGCUCAC | 17 | 532 |
| HSV1-UL19-100 | − | GCGGCCAUGGUGCCGAC | 17 | 520 |
| HSV1-UL19-132 | − | GAGGCCCUGGACGGGAC | 17 | 538 |
| HSV1-UL19-128 | − | GCUCACGGGGGAGGCCC | 17 | 534 |
| HSV1-UL19-137 | + | GGGCCUCCCCCGUGAGC | 17 | 541 |
| HSV1-UL19-158 | + | GGGGUGCGGGCCGUCGC | 17 | 431 |
| HSV1-UL19-145 | + | GGCUGAAGGCGGCGUUC | 17 | 549 |
| HSV1-UL19-114 | − | GCUGGCCUACAUGAACG | 17 | 526 |
| HSV1-UL19-180 | + | GGUCGGCACCAUGGCCG | 17 | 570 |
| HSV1-UL19-3221 | + | GCGGCCAUGGGGUCGCG | 17 | 7820 |
| HSV1-UL19-185 | + | GGUAUCCCGGAGGGUCG | 17 | 458 |
| HSV1-UL19-144 | + | GGUGGCCAGGCUGAAGG | 17 | 548 |
| HSV1-UL19-183 | + | GCAUACCGGUAUCCCGG | 17 | 572 |
| HSV1-UL19-111 | − | GGAGCUCGGGUUGUCGG | 17 | 406 |
| HSV1-UL19-161 | + | GGGCAAUCAGCGGCUGG | 17 | 434 |
| HSV1-UL19-3222 | + | GGUUGGGAGCGGCCAUG | 17 | 7821 |
| HSV1-UL19-154 | + | GUGUGGGUUGCUCGAUG | 17 | 556 |
| HSV1-UL19-155 | + | GGUUGCUCGAUGGGGUG | 17 | 557 |
| HSV1-UL19-150 | + | GGUCAUGUAAUUGUGUG | 17 | 554 |
| HSV1-UL19-170 | + | GUACAGGCUGUUUGCAU | 17 | 450 |
| HSV1-UL19-164 | + | GGCCAGCUCCGGAAACU | 17 | 444 |
| HSV1-UL19-140 | + | GAGCAGGGCGAUGGCCU | 17 | 544 |
| HSV1-UL19-108 | − | GUGCGCUUUCUGGAGCU | 17 | 401 |
| HSV1-UL19-162 | + | GCGCCCCUCGUUCAUGU | 17 | 561 |
| HSV1-UL19-151 | + | GUCAUGUAAUUGUGUGU | 17 | 555 |
| HSV1-UL19-21 | − | GAGCUGGCCUACAUGAACGA | 20 | 470 |
| HSV1-UL19-37 | − | GGGGGAGGCCCUGGACGGGA | 20 | 480 |
| HSV1-UL19-49 | + | GGCCUCGGUGGCCAGGCUGA | 20 | 490 |
| HSV1-UL19-30 | − | GAGGCCAUCGCCCUGCUCAC | 20 | 475 |
| HSV1-UL19-6 | − | GCCGCGGCCAUGGUGCCGAC | 20 | 463 |
| HSV1-UL19-38 | − | GGGGAGGCCCUGGACGGGAC | 20 | 481 |
| HSV1-UL19-83 | + | GAUCGUGCUAAGGAGGGACC | 20 | 510 |
| HSV1-UL19-2 | − | GCUCCCAACCGCGACCCUCC | 20 | 462 |
| HSV1-UL19-19 | − | GUGUACCAAGUUUCCGGAGC | 20 | 405 |
| HSV1-UL19-64 | + | GAUGGGGUGCGGGCCGUCGC | 20 | 429 |
| HSV1-UL19-20 | − | GGAGCUGGCCUACAUGAACG | 20 | 469 |
| HSV1-UL19-3223 | + | GGAGCGGCCAUGGGGUCGCG | 20 | 7822 |
| HSV1-UL19-47 | + | GAGCAGGGCGAUGGCCUCGG | 20 | 488 |
| HSV1-UL19-84 | + | GUGCUAAGGAGGGACCCGGU | 20 | 511 |
| HSV1-UL19-92 | + | GUAUCCCGGAGGGUCGCGGU | 20 | 456 |

Table 51D provides exemplary targeting domains for knocking out the UL19 gene selected according to the fourth tier parameters. The targeting domains bind within the first 500 bp of the coding sequence (e.g., within 500 bp downstream from the start codon). It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 5D
4th Tier

| | DNA | | Target | Seq ID |
|---|---|---|---|---|
| gRNA Name | Strand | Targeting Domain | Site Length | NO: |
| HSV1-UL19-139 | + | CCCCGUGAGCAGGGCGA | 17 | 543 |
| HSV1-UL19-129 | − | ACGGGGGAGGCCCUGGA | 17 | 535 |
| HSV1-UL19-143 | + | CUCGGUGGCCAGGCUGA | 17 | 547 |
| HSV1-UL19-130 | − | CGGGGGAGGCCCUGGAC | 17 | 536 |
| HSV1-UL19-101 | − | CGGCCAUGGUGCCGACC | 17 | 521 |
| HSV1-UL19-177 | + | CGUGCUAAGGAGGGACC | 17 | 567 |
| HSV1-UL19-120 | − | CAAGAUUAUCGACCGCC | 17 | 416 |
| HSV1-UL19-147 | + | AGGCGGCGUUCAGGGCC | 17 | 551 |
| HSV1-UL19-142 | + | CGAUGGCCUCGGUGGCC | 17 | 546 |
| HSV1-UL19-96 | − | CCCAACCGCGACCCUCC | 17 | 519 |
| HSV1-UL19-167 | + | UGCAAUACGACCCCAGC | 17 | 447 |
| HSV1-UL19-113 | − | UACCAAGUUUCCGGAGC | 17 | 408 |
| HSV1-UL19-95 | − | UCCCAACCGCGACCCUC | 17 | 518 |
| HSV1-UL19-583 | − | ACGUCCAGGCCGUCCUC | 17 | 969 |
| HSV1-UL19-163 | + | UUCAUGUAGGCCAGCUC | 17 | 443 |
| HSV1-UL19-109 | − | UGCGCUUUCUGGAGCUC | 17 | 402 |
| HSV1-UL19-112 | − | CGUGUGUACCAAGUUUC | 17 | 407 |
| HSV1-UL19-159 | + | CCGUCGCGGGCAAUCAG | 17 | 432 |
| HSV1-UL19-116 | − | UGGCCUACAUGAACGAG | 17 | 528 |
| HSV1-UL19-188 | + | AGGGUCGCGGUUGGGAG | 17 | 574 |
| HSV1-UL19-122 | − | CUUCAGCCUGGCCACCG | 17 | 530 |
| HSV1-UL19-584 | − | CGUCCAGGCCGUCCUCG | 17 | 970 |
| HSV1-UL19-157 | + | UGGGGUGCGGGCCGUCG | 17 | 559 |
| HSV1-UL19-148 | + | CGGCGUUCAGGGCCCGG | 17 | 552 |
| HSV1-UL19-141 | + | CAGGGCGAUGGCCUCGG | 17 | 545 |
| HSV1-UL19-127 | − | CGCCCUGCUCACGGGGG | 17 | 420 |
| HSV1-UL19-3224 | + | CGGUUGGGAGCGGCCAU | 17 | 7823 |
| HSV1-UL19-3225 | + | UGGGGUCGCGUGGAGAU | 17 | 7824 |
| HSV1-UL19-178 | + | CUAAGGAGGGACCCGGU | 17 | 568 |
| HSV1-UL19-186 | + | UCCCGGAGGGUCGCGGU | 17 | 459 |
| HSV1-UL19-110 | − | UCUGGAGCUCGGGUUGU | 17 | 525 |
| HSV1-UL19-44 | + | CAGGGCCUCCCCCGUGAGCA | 20 | 485 |
| HSV1-UL19-58 | + | AUUGUGUGUGGGUUGCUCGA | 20 | 425 |
| HSV1-UL19-35 | − | CUCACGGGGGAGGCCCUGGA | 20 | 478 |
| HSV1-UL19-79 | + | CGCGGGAAAAAAAAUCAAAC | 20 | 507 |
| HSV1-UL19-36 | − | UCACGGGGGAGGCCCUGGAC | 20 | 479 |
| HSV1-UL19-34 | − | CCUGCUCACGGGGGAGGCCC | 20 | 477 |
| HSV1-UL19-27 | − | CCUGAACGCCGCCUUCAGCC | 20 | 472 |
| HSV1-UL19-43 | + | CCAGGGCCUCCCCCGUGAGC | 20 | 484 |
| HSV1-UL19-22 | − | AGCUGGCCUACAUGAACGAG | 20 | 471 |
| HSV1-UL19-94 | + | CGGAGGGUCGCGGUUGGGAG | 20 | 517 |
| HSV1-UL19-28 | − | CGCCUUCAGCCUGGCCACCG | 20 | 473 |
| HSV1-UL19-50 | + | CUCGGUGGCCAGGCUGAAGG | 20 | 491 |
| HSV1-UL19-56 | + | CUUGGUCAUGUAAUUGUGUG | 20 | 497 |
| HSV1-UL19-3226 | + | CCAUGGGGUCGCGUGGAGAU | 20 | 7825 |
| HSV1-UL19-46 | + | CGUGAGCAGGGCGAUGGCCU | 20 | 487 |
| HSV1-UL19-57 | + | UUGGUCAUGUAAUUGUGUGU | 20 | 498 |

Table 5E provides exemplary targeting domains for knocking out the UL19 gene selected according to the fifth tier parameters. The targeting domains fall in the coding sequence of the gene, downstream of the first 500 bp of coding sequence (e.g., anywhere from +500 (relative to the start codon) to the stop codon of the gene). It is contemplated herein that in an embodiment the targeting domain hybridizes to the target domain through complementary base pairing. Any of the targeting domains in the table can be used with a S. pyogenes Cas9 molecule that generates a double stranded break (Cas9 nuclease) or a single-stranded break (Cas9 nickase).

TABLE 5E
5th Tier

| | DNA | | Target Site | Seq ID |
|---|---|---|---|---|
| gRNA Name | Strand | Targeting Domain | Length | NO: |
| HSV1-UL19-838 | − | GACCAUACCAUCCAAAA | 17 | 1224 |
| HSV1-UL19-588 | − | GCACGUGCUGCUGGAAA | 17 | 974 |
| HSV1-UL19-827 | − | CAUGGCCGAACGCACAA | 17 | 1213 |
| HSV1-UL19-818 | − | AACGCCAUGUUUCACAA | 17 | 1204 |
| HSV1-UL19-591 | − | CAACGAUACCUGGACAA | 17 | 977 |
| HSV1-UL19-967 | − | CUCCGCAGUGCCCGCAA | 17 | 1353 |
| HSV1-UL19-947 | − | GGGGCCUACCACCUCAA | 17 | 1333 |
| HSV1-UL19-973 | − | GACGCAUCCCCGCUCAA | 17 | 1359 |
| HSV1-UL19-1554 | + | CAACGUUCAUCAGCGAA | 17 | 1940 |
| HSV1-UL19-1617 | + | GUCGAACGCCCCGCGAA | 17 | 2003 |
| HSV1-UL19-655 | − | UCAAUCCGGUCAUGGAA | 17 | 1041 |
| HSV1-UL19-624 | − | GGCGAGAUGGUCCUGAA | 17 | 1010 |
| HSV1-UL19-720 | − | ACCUGGCGCGUGGUGAA | 17 | 1106 |
| HSV1-UL19-944 | − | GGGGACCUGCUCUAUAA | 17 | 1330 |
| HSV1-UL19-1712 | + | GCGCCCGUUGUGAAACA | 17 | 2098 |
| HSV1-UL19-826 | − | GGUGCUGGCCCACAACA | 17 | 1212 |
| HSV1-UL19-817 | − | CAAUCUGGUGGCCAACA | 17 | 1203 |
| HSV1-UL19-808 | − | CGCCACCCUGCAGAACA | 17 | 1194 |
| HSV1-UL19-1823 | + | CUGGGCGCACCCGAACA | 17 | 2209 |
| HSV1-UL19-1829 | + | GGCGAUAAACUCACACA | 17 | 2215 |
| HSV1-UL19-1886 | + | UCCUGAAACAGGCCACA | 17 | 2272 |
| HSV1-UL19-1552 | + | CAGCGAAGGGUGGCACA | 17 | 1938 |
| HSV1-UL19-890 | − | CAACCCCGUCACCGACA | 17 | 1276 |
| HSV1-UL19-1834 | + | GUUGACGUCGGUCGACA | 17 | 2220 |
| HSV1-UL19-929 | − | CGUUUACGCGGGGGACA | 17 | 1315 |
| HSV1-UL19-743 | − | CUGCAGGCCGCCAUACA | 17 | 1129 |
| HSV1-UL19-1665 | + | ACAUGCCGCCGCGUACA | 17 | 2051 |
| HSV1-UL19-1812 | + | CGCGUUCCGCAGGUACA | 17 | 2198 |
| HSV1-UL19-1635 | + | CACCAGGUCCCGGUACA | 17 | 2021 |
| HSV1-UL19-1516 | + | GUCCAUGGCGCCCACCA | 17 | 1902 |
| HSV1-UL19-594 | − | CGGCCGCCUGGCCACCA | 17 | 980 |
| HSV1-UL19-610 | − | GCUCGUGGACCUCACCA | 17 | 996 |
| HSV1-UL19-934 | − | GCCCUCAUGUACGACCA | 17 | 1320 |
| HSV1-UL19-1459 | + | CUUUAGCUCGGCGACCA | 17 | 1845 |
| HSV1-UL19-1759 | + | CAGAGCCGGGGGGACCA | 17 | 2145 |
| HSV1-UL19-915 | − | CGCGCAGGGAUGGACCA | 17 | 1301 |
| HSV1-UL19-1859 | + | AAUCGCUGCGACGCCCA | 17 | 2245 |
| HSV1-UL19-1522 | + | CGGAUUGAACAGGCCCA | 17 | 1908 |
| HSV1-UL19-688 | − | GGGCAGCGCCUGGCCCA | 17 | 1074 |
| HSV1-UL19-1449 | + | CGGCAACAACAAAGCCA | 17 | 1835 |
| HSV1-UL19-736 | − | GGUGGGACGCCACGCCA | 17 | 1122 |
| HSV1-UL19-1601 | + | CGCCGGACAUAGCGCCA | 17 | 1987 |
| HSV1-UL19-650 | − | CCCCCUGGUGGGCGCCA | 17 | 1036 |
| HSV1-UL19-1774 | + | CUGAUGAUGCAAGGCCA | 17 | 2160 |
| HSV1-UL19-1473 | + | CAGACGGGGCACGGCCA | 17 | 1859 |
| HSV1-UL19-938 | − | CCGGGCCUUCGCGGCCA | 17 | 1324 |
| HSV1-UL19-1624 | + | GACCACGAGCCGGGCCA | 17 | 2010 |
| HSV1-UL19-1609 | + | GAUGGUGGCGGGGGCCA | 17 | 1995 |
| HSV1-UL19-1638 | + | AUCAACCAGCUGGGCCA | 17 | 2024 |
| HSV1-UL19-889 | − | CACCGCCGUCGUGGCCA | 17 | 1275 |
| HSV1-UL19-1507 | + | GUAGAUGCGCUUCUCCA | 17 | 1893 |
| HSV1-UL19-1518 | + | GACGAACGUCAGGUCCA | 17 | 1904 |
| HSV1-UL19-1657 | + | GUCGCGAUGGCGGUCCA | 17 | 2043 |
| HSV1-UL19-958 | − | GGGUUCGGCGGUGUCCA | 17 | 1344 |
| HSV1-UL19-1867 | + | GUAAAGAACUUAAAGCA | 17 | 2253 |
| HSV1-UL19-1715 | + | CACCUGCAGCGUGAGCA | 17 | 2101 |
| HSV1-UL19-913 | − | AGGUGCCGCGGCGCGCA | 17 | 1299 |
| HSV1-UL19-1754 | + | CCGCGACAGGUCGCGCA | 17 | 2140 |
| HSV1-UL19-1495 | + | GUCGUCCAGACUUCGCA | 17 | 1881 |
| HSV1-UL19-586 | − | GGCGUUUGAGCGCGGCA | 17 | 972 |
| HSV1-UL19-1474 | + | AUGCGUCAGACGGGGCA | 17 | 1860 |
| HSV1-UL19-631 | − | GGCGCUCGUGAUGGGCA | 17 | 1017 |
| HSV1-UL19-601 | − | GAGCUUUUUCCUGGGCA | 17 | 987 |
| HSV1-UL19-1775 | + | CUUGAGCUGAUGAUGCA | 17 | 2161 |
| HSV1-UL19-1687 | + | GACCACCAUGUUCUGCA | 17 | 2073 |
| HSV1-UL19-756 | − | CCUUCCCGAGGAGUGCA | 17 | 1142 |
| HSV1-UL19-1706 | + | CAGAUUGGCCGGGUGCA | 17 | 2092 |
| HSV1-UL19-1678 | + | GUAAAUCUUGUGGUGCA | 17 | 2064 |
| HSV1-UL19-1838 | + | CGUCCCCGGGGGUUGCA | 17 | 2224 |
| HSV1-UL19-1655 | + | GGCCGCGCGCCGCAUCA | 17 | 2041 |
| HSV1-UL19-972 | − | UGACGCAUCCCCGCUCA | 17 | 1358 |
| HSV1-UL19-856 | − | GCUGACCUACGCGCUCA | 17 | 1242 |
| HSV1-UL19-1700 | + | GUGCGCGGGGUCCGUCA | 17 | 2086 |
| HSV1-UL19-1642 | + | CAGCUCCGGGCCGGUCA | 17 | 2028 |
| HSV1-UL19-654 | − | CCUGUUCAAUCCGGUCA | 17 | 1040 |
| HSV1-UL19-628 | − | CGGGGCCAACCUGGUCA | 17 | 1014 |
| HSV1-UL19-861 | − | GCAUCAUCAGCUCAAGA | 17 | 1247 |
| HSV1-UL19-1477 | + | UGUCGGCAUGCGUCAGA | 17 | 1863 |
| HSV1-UL19-1682 | + | GCAGCGGCCCCGCGAGA | 17 | 2068 |
| HSV1-UL19-623 | − | GGUGACGUACGGCGAGA | 17 | 1009 |
| HSV1-UL19-1809 | + | CCGCGUUGUCCAGGAGA | 17 | 2195 |
| HSV1-UL19-954 | − | GCGCCUGAUCGUGGAGA | 17 | 1340 |
| HSV1-UL19-788 | − | GACUUCAACCGCAACGA | 17 | 1174 |
| HSV1-UL19-1653 | + | GUCACAGUCCCACACGA | 17 | 2039 |
| HSV1-UL19-1793 | + | GGUUGCCGUGGCCACGA | 17 | 2179 |
| HSV1-UL19-1769 | + | GCGGACGUGCUGCACGA | 17 | 2155 |
| HSV1-UL19-1780 | + | GCGGUCCUGUCGGACGA | 17 | 2166 |
| HSV1-UL19-1506 | + | GACCAGCUUCUCGCCGA | 17 | 1892 |
| HSV1-UL19-692 | − | CCCACGGGCGGGUCCGA | 17 | 1078 |
| HSV1-UL19-1555 | + | UCAACGUUCAUCAGCGA | 17 | 1941 |
| HSV1-UL19-1855 | + | GUUGGCCGUGGCCGCGA | 17 | 2241 |
| HSV1-UL19-1660 | + | UAACCCGGCAGUCGCGA | 17 | 2046 |
| HSV1-UL19-1615 | + | CCCGCGAACGGCGGCGA | 17 | 2001 |
| HSV1-UL19-1466 | + | CACGAGCCAGGCCUCGA | 17 | 1852 |
| HSV1-UL19-616 | − | CGCGGGCGGCCGGUCGA | 17 | 1002 |
| HSV1-UL19-832 | − | AACAUGCGCAUAUUCGA | 17 | 1218 |
| HSV1-UL19-1560 | + | CGGAUUGGCGGCUUCGA | 17 | 1946 |
| HSV1-UL19-931 | − | UACGCGGGGGACAAGGA | 17 | 1317 |
| HSV1-UL19-1487 | + | GUCCUCCACUUUCAGGA | 17 | 1873 |
| HSV1-UL19-1443 | + | CUCAAACGCCCCGAGGA | 17 | 1829 |
| HSV1-UL19-1896 | + | GCGGGCACUGCGGAGGA | 17 | 2282 |
| HSV1-UL19-1471 | + | CGGGGCACGGCCACGGA | 17 | 1857 |
| HSV1-UL19-695 | − | GUCCGAUGGGUCGCGGA | 17 | 1081 |
| HSV1-UL19-1795 | + | GGUGACGGGGUUGCGGA | 17 | 2181 |
| HSV1-UL19-914 | − | GCCGCGGCGCGCAGGGA | 17 | 1300 |
| HSV1-UL19-1777 | + | UGAACCCAAACCCGGGA | 17 | 2163 |
| HSV1-UL19-1852 | + | GCCGCGAAGGCCCGGGA | 17 | 2238 |
| HSV1-UL19-1542 | + | CAGCUGGCGGGGCGGGA | 17 | 1928 |
| HSV1-UL19-1632 | + | CCAUGCACUCCUCGGGA | 17 | 2018 |
| HSV1-UL19-1820 | + | GCACCCGAACACGGGGA | 17 | 2206 |
| HSV1-UL19-1738 | + | CAGCGCGUGGACGGGGA | 17 | 2124 |
| HSV1-UL19-665 | − | GCUGUUUUUUUGGGGGA | 17 | 1051 |
| HSV1-UL19-1801 | + | GGUAAAAGUUUUGGGGA | 17 | 2187 |
| HSV1-UL19-822 | − | GGGCGCGUGGUAGUGGA | 17 | 1208 |
| HSV1-UL19-1742 | + | GGCGAACAGCGCGUGGA | 17 | 2128 |
| HSV1-UL19-3209 | + | GCGAGCGAGCUGUUGGA | 17 | 7808 |
| HSV1-UL19-1734 | + | AUAGUCGCCAUUUUGGA | 17 | 2120 |
| HSV1-UL19-1847 | + | GCCGUGGUCGUACAUGA | 17 | 2233 |
| HSV1-UL19-836 | − | CGCCGGAAUCCUGCUGA | 17 | 1222 |
| HSV1-UL19-800 | − | GAUUUACUACUACGUGA | 17 | 1186 |
| HSV1-UL19-813 | − | CCCCAGCGACCCCGUGA | 17 | 1199 |
| HSV1-UL19-629 | − | GGUCACGGCGCUCGUGA | 17 | 1015 |
| HSV1-UL19-1799 | + | GUUGCCCAUGUCGGUGA | 17 | 2185 |
| HSV1-UL19-622 | − | GACGUGCCGGUGACGUA | 17 | 1008 |
| HSV1-UL19-1563 | + | GCCACGUACGCCCCGUA | 17 | 1949 |
| HSV1-UL19-676 | − | GAAGCCGCCAAUCCGUA | 17 | 1062 |
| HSV1-UL19-941 | − | UCGCAGCGAUUUUCGUA | 17 | 1327 |
| HSV1-UL19-1512 | + | GCGCCCACCAGGGGGUA | 17 | 1898 |
| HSV1-UL19-1733 | + | CGCCAUUUUGGAUGGUA | 17 | 2119 |
| HSV1-UL19-1623 | + | GUGCUCGCUGCCGUGUA | 17 | 2009 |
| HSV1-UL19-1556 | + | CGACGGGGUCGCGGUUA | 17 | 1942 |
| HSV1-UL19-876 | − | GUGGCCCGGCACGAAAC | 17 | 1262 |
| HSV1-UL19-1887 | + | GGUAGGCCUCCUGAAAC | 17 | 2273 |
| HSV1-UL19-819 | − | ACGCCAUGUUUCACAAC | 17 | 1205 |
| HSV1-UL19-968 | − | UCCGCAGUGCCCGCAAC | 17 | 1354 |
| HSV1-UL19-134 | + | GUUGGAUGGCGCGCAAC | 17 | 422 |
| HSV1-UL19-948 | − | GGGCCUACCACCUCAAC | 17 | 1334 |
| HSV1-UL19-638 | − | AGCAGCUCGACCUGAAC | 17 | 1024 |
| HSV1-UL19-625 | − | GCGAGAUGGUCCUGAAC | 17 | 1011 |
| HSV1-UL19-1524 | + | CCAUGACCGGAUUGAAC | 17 | 1910 |
| HSV1-UL19-945 | − | GGGACCUGCUCUAUAAC | 17 | 1331 |
| HSV1-UL19-1822 | + | UGGGCGCACCCGAACAC | 17 | 2208 |
| HSV1-UL19-1885 | + | CCUGAAACAGGCCACAC | 17 | 2271 |
| HSV1-UL19-1833 | + | UUGACGUCGGUCGACAC | 17 | 2219 |
| HSV1-UL19-1664 | + | CAUGCCGCCGCGUACAC | 17 | 2050 |
| HSV1-UL19-1858 | + | AUCGCUGCGACGCCCAC | 17 | 2244 |
| HSV1-UL19-689 | − | GGCAGCGCCUGGCCCAC | 17 | 1075 |
| HSV1-UL19-1773 | + | UGAUGAUGCAAGGCCAC | 17 | 2159 |
| HSV1-UL19-1865 | + | AAGCAGGGGCUGAGCAC | 17 | 2251 |
| HSV1-UL19-793 | − | CGGAUGACCGGCCGCAC | 17 | 1179 |
| HSV1-UL19-1628 | + | AGUAGCUGGUGAUGCAC | 17 | 2014 |
| HSV1-UL19-1491 | + | AUCUCGCCGUACGUCAC | 17 | 1877 |
| HSV1-UL19-1699 | + | UGCGCGGGGUCCGUCAC | 17 | 2085 |
| HSV1-UL19-862 | − | CAUCAUCAGCUCAAGAC | 17 | 1248 |
| HSV1-UL19-1476 | + | GUCGGCAUGCGUCAGAC | 17 | 1862 |
| HSV1-UL19-1551 | + | UCGCGUGUUCCAGAGAC | 17 | 1937 |
| HSV1-UL19-955 | − | CGCCUGAUCGUGGAGAC | 17 | 1341 |
| HSV1-UL19-1768 | + | CGGACGUGCUGCACGAC | 17 | 2154 |
| HSV1-UL19-867 | − | GUUCACCGUCGUCCGAC | 17 | 1253 |
| HSV1-UL19-1756 | + | GGGGGACCUGCCGCGAC | 17 | 2142 |
| HSV1-UL19-1479 | + | ACGAGCACCCCGUCGAC | 17 | 1865 |
| HSV1-UL19-617 | − | GCGGGCGGCCGGUCGAC | 17 | 1003 |
| HSV1-UL19-833 | − | ACAUGCGCAUAUUCGAC | 17 | 1219 |
| HSV1-UL19-1559 | + | GGAUUGGCGGCUUCGAC | 17 | 1945 |
| HSV1-UL19-799 | − | CGCACCGGGGGGCGGAC | 17 | 1185 |
| HSV1-UL19-974 | − | AUCCCCGCUCAAGGGAC | 17 | 1360 |
| HSV1-UL19-1851 | + | CCGCGAAGGCCCGGGAC | 17 | 2237 |
| HSV1-UL19-1819 | + | CACCCGAACACGGGGAC | 17 | 2205 |
| HSV1-UL19-823 | − | GGCGCGUGGUAGUGGAC | 17 | 1209 |
| HSV1-UL19-1741 | + | GCGAACAGCGCGUGGAC | 17 | 2127 |
| HSV1-UL19-1525 | + | GCAAACCGUUCCAUGAC | 17 | 1911 |
| HSV1-UL19-792 | − | CGGACGCCGCGGAUGAC | 17 | 1178 |
| HSV1-UL19-764 | − | GAUGACUUUACCCUGAC | 17 | 1150 |
| HSV1-UL19-1798 | + | UUGCCCAUGUCGGUGAC | 17 | 2184 |
| HSV1-UL19-747 | − | AGUGCAUCACCAGCUAC | 17 | 1133 |
| HSV1-UL19-677 | − | AAGCCGCCAAUCCGUAC | 17 | 1063 |
| HSV1-UL19-942 | − | CGCAGCGAUUUUCGUAC | 17 | 1328 |
| HSV1-UL19-757 | − | AGUGCAUGGCCGUGUAC | 17 | 1143 |
| HSV1-UL19-1779 | + | GACGGUGAACCCAAACC | 17 | 2165 |
| HSV1-UL19-697 | − | GCCCGACAACGCCAACC | 17 | 1083 |
| HSV1-UL19-627 | − | CCUGAACGGGGCCAACC | 17 | 1013 |
| HSV1-UL19-1661 | + | GGCCCCCCGCGCUAACC | 17 | 2047 |
| HSV1-UL19-1517 | + | GGUCCAUGGCGCCCACC | 17 | 1903 |
| HSV1-UL19-1637 | + | CCUCGACGUGCGCCACC | 17 | 2023 |
| HSV1-UL19-718 | − | GGGAGAUCCAGGCCACC | 17 | 1104 |
| HSV1-UL19-660 | − | CCCCGCCCCCGGCCACC | 17 | 1046 |
| HSV1-UL19-1785 | + | UUUCGUGCCGGGCCACC | 17 | 2171 |
| HSV1-UL19-593 | − | ACGGCCGCCUGGCCACC | 17 | 979 |
| HSV1-UL19-1550 | + | GAGACAGGCGCAGCACC | 17 | 1936 |
| HSV1-UL19-794 | − | GGAUGACCGGCCGCACC | 17 | 1180 |
| HSV1-UL19-1825 | + | CUGCGCGCCGCGGCACC | 17 | 2211 |
| HSV1-UL19-814 | − | CGCGCACCCCCUGCACC | 17 | 1200 |
| HSV1-UL19-771 | − | UCACCUAAUGCGAGACC | 17 | 1157 |
| HSV1-UL19-1460 | + | GCUUUAGCUCGGCGACC | 17 | 1846 |
| HSV1-UL19-759 | − | GGCCGUGUACCGGGACC | 17 | 1145 |
| HSV1-UL19-1760 | + | CCAGAGCCGGGGGGACC | 17 | 2146 |
| HSV1-UL19-884 | − | GCGCGCGAACGUGGACC | 17 | 1270 |
| HSV1-UL19-1494 | + | UCACGAGCGCCGUGACC | 17 | 1880 |
| HSV1-UL19-590 | − | GCCGAUGCAACGAUACC | 17 | 976 |
| HSV1-UL19-740 | − | CGACGACCGCAACUACC | 17 | 1126 |
| HSV1-UL19-758 | − | GUGCAUGGCCGUGUACC | 17 | 1144 |
| HSV1-UL19-892 | − | UCCCCAAAACUUUUACC | 17 | 1278 |
| HSV1-UL19-1778 | + | ACGGUGAACCCAAACCC | 17 | 2164 |
| HSV1-UL19-789 | − | GCUGCUGCACAACACCC | 17 | 1175 |
| HSV1-UL19-1456 | + | GGUCGCCCGGGCCACCC | 17 | 1842 |
| HSV1-UL19-1883 | + | ACGAGUUCGCGGCACCC | 17 | 2269 |
| HSV1-UL19-598 | − | GGUGGCCCGGGCGACCC | 17 | 984 |
| HSV1-UL19-696 | − | GGAAGGCCAGAUGACCC | 17 | 1082 |
| HSV1-UL19-918 | − | GCCGGCCCUGCAACCCC | 17 | 1304 |
| HSV1-UL19-1882 | + | CGAGUUCGCGGCACCCC | 17 | 2268 |
| HSV1-UL19-919 | − | CCGGCCCUGCAACCCCC | 17 | 1305 |
| HSV1-UL19-647 | − | CAACGUUCCCUACCCCC | 17 | 1033 |
| HSV1-UL19-1589 | + | GGGGGAACGUCCCCCCC | 17 | 1975 |
| HSV1-UL19-960 | − | CAGUUUAAGCGCCCCCC | 17 | 1346 |
| HSV1-UL19-1677 | + | CGGUCCAGUCCGCCCCC | 17 | 2063 |
| HSV1-UL19-959 | − | ACAGUUUAAGCGCCCCC | 17 | 1345 |
| HSV1-UL19-1572 | + | GGCCAGGCGCUGCCCCC | 17 | 1958 |
| HSV1-UL19-1595 | + | UGGCCUGGAUCUCCCCC | 17 | 1981 |
| HSV1-UL19-846 | − | GUCGCGGCAGGUCCCCC | 17 | 1232 |
| HSV1-UL19-847 | − | GGUCCCCCUGGUCCCCC | 17 | 1233 |
| HSV1-UL19-843 | − | CGCGCCCAAUUUCCCCC | 17 | 1229 |
| HSV1-UL19-1845 | + | CCCCGCGUAAACGCCCC | 17 | 2231 |
| HSV1-UL19-659 | − | GACCUAGUCCCCGCCCC | 17 | 1045 |
| HSV1-UL19-589 | − | GGAAAAGGCGCCGCCCC | 17 | 975 |
| HSV1-UL19-811 | − | GUCCCGGAGAUCGCCCC | 17 | 1197 |
| HSV1-UL19-1605 | + | CCCCAGCUCCAGGCCCC | 17 | 1991 |
| HSV1-UL19-1815 | + | CGGGCUGGGCCGGCCCC | 17 | 2201 |
| HSV1-UL19-651 | − | GACGUUCGUCCUGCCCC | 17 | 1037 |
| HSV1-UL19-721 | − | GAACGGCAACUUGCCCC | 17 | 1107 |
| HSV1-UL19-1596 | + | GUGGCCUGGAUCUCCCC | 17 | 1982 |
| HSV1-UL19-1842 | + | CCCGGCGGCGCGUCCCC | 17 | 2228 |
| HSV1-UL19-711 | − | GGGGGGGGACGUUCCCC | 17 | 1097 |
| HSV1-UL19-1791 | + | CGGUGUAGCCCACGCCC | 17 | 2177 |
| HSV1-UL19-727 | − | GGCGUUCCGGGACGCCC | 17 | 1113 |
| HSV1-UL19-744 | − | GCACGUCUUCUGCGCCC | 17 | 1130 |
| HSV1-UL19-910 | − | CGUGUUCGGGUGCGCCC | 17 | 1296 |
| HSV1-UL19-1457 | + | GGCGACCAGGGUCGCCC | 17 | 1843 |
| HSV1-UL19-1853 | + | CGUGGCCGCGAAGGCCC | 17 | 2239 |
| HSV1-UL19-1523 | + | CCGGAUUGAACAGGCCC | 17 | 1909 |
| HSV1-UL19-1606 | + | CCCCCAGCUCCAGGCCC | 17 | 1992 |
| HSV1-UL19-762 | − | GGCGCACGUCGAGGCCC | 17 | 1148 |
| HSV1-UL19-646 | − | GGUCUUUCUGGAGGCCC | 17 | 1032 |
| HSV1-UL19-714 | − | GUUCCCCCGGCCGGCCC | 17 | 1100 |
| HSV1-UL19-1805 | + | GGGGAGCCCCGCGGCCC | 17 | 2191 |
| HSV1-UL19-776 | − | GAUGCGGCGCGCGGCCC | 17 | 1162 |
| HSV1-UL19-681 | − | GGCGUACGUGGCGGCCC | 17 | 1067 |
| HSV1-UL19-1744 | + | CGCCACGUGGUCGGCCC | 17 | 2130 |
| HSV1-UL19-1783 | + | CCACCUGGAGCUGGCCC | 17 | 2169 |
| HSV1-UL19-597 | − | GGCCACCAGGGUGGCCC | 17 | 983 |
| HSV1-UL19-1462 | + | GGUGGCCCGCCUUGCCC | 17 | 1848 |
| HSV1-UL19-864 | − | AGACGGGCCUCCAUCCC | 17 | 1250 |
| HSV1-UL19-1771 | + | GGUCAGCGCGUUCUCCC | 17 | 2157 |
| HSV1-UL19-936 | − | CCAGAGCGACCCGUCCC | 17 | 1322 |
| HSV1-UL19-1843 | + | CCCCGGCGGCGCGUCCC | 17 | 2229 |
| HSV1-UL19-1527 | + | GGCGGGGACUAGGUCCC | 17 | 1913 |
| HSV1-UL19-1450 | + | UCGGCAACAACAAAGCC | 17 | 1836 |
| HSV1-UL19-1584 | + | GGUGCAGCUCAAGAGCC | 17 | 1970 |
| HSV1-UL19-1764 | + | AGUUGGCCCCCAGAGCC | 17 | 2150 |
| HSV1-UL19-1626 | + | GCACUGGACCACGAGCC | 17 | 2012 |
| HSV1-UL19-1467 | + | GGUGAGGUCCACGAGCC | 17 | 1853 |
| HSV1-UL19-683 | − | AGCUGUUUUUGAACGCC | 17 | 1069 |
| HSV1-UL19-657 | − | GGUUUGCCGCGCACGCC | 17 | 1043 |
| HSV1-UL19-726 | − | CGGCGUUCCGGGACGCC | 17 | 1112 |
| HSV1-UL19-904 | − | CGUGGCGGGAAACCGCC | 17 | 1290 |
| HSV1-UL19-1619 | + | GCAGGUAAAACACCGCC | 17 | 2005 |
| HSV1-UL19-605 | − | CAAGGCGGGCCACCGCC | 17 | 991 |
| HSV1-UL19-666 | − | UUGGGGGAAGGACCGCC | 17 | 1052 |
| HSV1-UL19-922 | − | CCCGGGGACGCGCCGCC | 17 | 1308 |
| HSV1-UL19-592 | − | CCUGGACAACGGCCGCC | 17 | 978 |
| HSV1-UL19-1649 | + | GGGGUGGCAGCAGCGCC | 17 | 2035 |
| HSV1-UL19-687 | − | CGCCUGGGGGCAGCGCC | 17 | 1073 |
| HSV1-UL19-1602 | + | CCGCCGGACAUAGCGCC | 17 | 1988 |
| HSV1-UL19-1599 | + | GCCGUUCACCACGCGCC | 17 | 1985 |
| HSV1-UL19-1693 | + | CUGGGGCACUCCUCGCC | 17 | 2079 |
| HSV1-UL19-1458 | + | CGGCGACCAGGGUCGCC | 17 | 1844 |
| HSV1-UL19-840 | − | UCCACGCGCUGUUCGCC | 17 | 1226 |
| HSV1-UL19-1854 | + | CCGUGGCCGCGAAGGCC | 17 | 2240 |
| HSV1-UL19-608 | − | GGGAGGCCGUCGAGGCC | 17 | 994 |
| HSV1-UL19-916 | − | AGGGAUGGACCACGGCC | 17 | 1302 |
| HSV1-UL19-1442 | + | ACGCCCCGAGGACGGCC | 17 | 1828 |
| HSV1-UL19-765 | − | CUUUACCCUGACCGGCC | 17 | 1151 |
| HSV1-UL19-713 | − | CGUUCCCCCGGCCGGCC | 17 | 1099 |
| HSV1-UL19-1592 | + | UCUCCCCCGGGCCGGCC | 17 | 1978 |
| HSV1-UL19-1565 | + | CUGCGGCGGGGCCGGCC | 17 | 1951 |
| HSV1-UL19-854 | − | UCCGCGAGAGCGCGGCC | 17 | 1240 |
| HSV1-UL19-1752 | + | ACAGGUCGCGCAGGGCC | 17 | 2138 |
| HSV1-UL19-1625 | + | GGACCACGAGCCGGGCC | 17 | 2011 |
| HSV1-UL19-1670 | + | CGUCCGCGGCUCGGGCC | 17 | 2056 |
| HSV1-UL19-729 | − | CCGGGACGCCCGGGGCC | 17 | 1115 |
| HSV1-UL19-1639 | + | CAUCAACCAGCUGGGCC | 17 | 2025 |
| HSV1-UL19-1573 | + | GGACCCGCCCGUGGGCC | 17 | 1959 |
| HSV1-UL19-745 | − | UCUUCUGCGCCCUGGCC | 17 | 1131 |
| HSV1-UL19-1597 | + | CCACGCGCCAGGUGGCC | 17 | 1983 |
| HSV1-UL19-875 | − | GCCAGCUCCAGGUGGCC | 17 | 1261 |
| HSV1-UL19-596 | − | UGGCCACCAGGGUGGCC | 17 | 982 |
| HSV1-UL19-1534 | + | CGGGGAUCCGGGUGGCC | 17 | 1920 |
| HSV1-UL19-1454 | + | GGGCCACCCUGGUGGCC | 17 | 1840 |
| HSV1-UL19-1708 | + | UGGCCACCAGAUUGGCC | 17 | 2094 |
| HSV1-UL19-778 | − | CCGCCAUCGCGACUGCC | 17 | 1164 |
| HSV1-UL19-705 | − | GCCGACGUGGAGCUGCC | 17 | 1091 |
| HSV1-UL19-952 | − | CGCCAAACAUCGCUGCC | 17 | 1338 |
| HSV1-UL19-1786 | + | CCCGCCAGUUUCGUGCC | 17 | 2172 |
| HSV1-UL19-863 | − | AAGACGGGCCUCCAUCC | 17 | 1249 |
| HSV1-UL19-717 | − | CGGCCCGGGGGAGAUCC | 17 | 1103 |
| HSV1-UL19-1537 | + | GGAAGGCGCGGGGAUCC | 17 | 1923 |
| HSV1-UL19-1465 | + | AGGCCUCGACGGCCUCC | 17 | 1851 |
| HSV1-UL19-1607 | + | GUCCCACCCCCAGCUCC | 17 | 1993 |
| HSV1-UL19-1645 | + | CCUGCCCGCCCAGCUCC | 17 | 2031 |
| HSV1-UL19-873 | − | CUUCCUGGGCCAGCUCC | 17 | 1259 |
| HSV1-UL19-1872 | + | CCACGAUCAGGCGCUCC | 17 | 2258 |
| HSV1-UL19-1579 | + | GCUGCAUAAACUGCUCC | 17 | 1965 |
| HSV1-UL19-1689 | + | CGCCGGGGGCGAUCUCC | 17 | 2075 |
| HSV1-UL19-897 | − | CGGGGCUCCCCCUCUCC | 17 | 1283 |
| HSV1-UL19-1508 | + | CGUAGAUGCGCUUCUCC | 17 | 1894 |
| HSV1-UL19-3210 | − | GCUCGCUCGCAACGUCC | 17 | 7809 |
| HSV1-UL19-935 | − | GCCAGAGCGACCCGUCC | 17 | 1321 |
| HSV1-UL19-1828 | + | ACUCACACACGGCGUCC | 17 | 2214 |
| HSV1-UL19-1604 | + | CCAGGCCCCGGGCGUCC | 17 | 1990 |
| HSV1-UL19-1636 | + | CGUGCGCCACCAGGUCC | 17 | 2022 |
| HSV1-UL19-1658 | + | AGUCGCGAUGGCGGUCC | 17 | 2044 |
| HSV1-UL19-810 | − | GCAGAACAUGGUGGUCC | 17 | 1196 |
| HSV1-UL19-1452 | + | CCAGGCGGCCGUUGUCC | 17 | 1838 |
| HSV1-UL19-1811 | + | CGGCUGCCGCGUUGUCC | 17 | 2197 |
| HSV1-UL19-871 | − | GUCGGAGGCGUACUUCC | 17 | 1257 |
| HSV1-UL19-725 | − | AUGUCCGGCGGCGUUCC | 17 | 1111 |
| HSV1-UL19-599 | − | CGAGACGAGCUUUUUCC | 17 | 985 |
| HSV1-UL19-1868 | + | CGUAAAGAACUUAAAGC | 17 | 2254 |
| HSV1-UL19-643 | − | GUCCAUCGGCGAGAAGC | 17 | 1029 |
| HSV1-UL19-1548 | + | UCCCCCAAAAAAACAGC | 17 | 1934 |
| HSV1-UL19-1641 | + | UAAAGUCAUCAACCAGC | 17 | 2027 |
| HSV1-UL19-763 | − | CGAGGCCCUGGCCCAGC | 17 | 1149 |
| HSV1-UL19-1498 | + | CCUCCUGCAUCUCCAGC | 17 | 1884 |
| HSV1-UL19-1668 | + | GGGUGUUGUGCAGCAGC | 17 | 2054 |
| HSV1-UL19-1621 | + | GUAUGGCGGCCUGCAGC | 17 | 2007 |
| HSV1-UL19-1727 | + | GCUGGGGGGCCAUCAGC | 17 | 2113 |
| HSV1-UL19-1765 | + | UAGUUGGCCCCCAGAGC | 17 | 2151 |
| HSV1-UL19-1860 | + | AGGCCCCGUUAUAGAGC | 17 | 2246 |
| HSV1-UL19-1627 | + | UGCACUGGACCACGAGC | 17 | 2013 |
| HSV1-UL19-766 | − | CCUGACCGGCCCGGAGC | 17 | 1152 |
| HSV1-UL19-1784 | + | GCCGGGCCACCUGGAGC | 17 | 2170 |
| HSV1-UL19-730 | − | CGCCCGGGGCCUGGAGC | 17 | 1116 |
| HSV1-UL19-1889 | + | CGCUGGCGCAGGUGAGC | 17 | 2275 |
| HSV1-UL19-1907 | + | GAGCCAGUCCCUUGAGC | 17 | 2293 |
| HSV1-UL19-1629 | + | CGUGUUGUUCCAGUAGC | 17 | 2015 |
| HSV1-UL19-1586 | + | ACAAAGAAGUCGAACGC | 17 | 1972 |
| HSV1-UL19-656 | − | CGGUUUGCCGCGCACGC | 17 | 1042 |
| HSV1-UL19-835 | − | GACGGGGCGUUGCACGC | 17 | 1221 |
| HSV1-UL19-639 | − | CCUGAACCGGCAGACGC | 17 | 1025 |
| HSV1-UL19-831 | − | UCGGCGGCGCCCGACGC | 17 | 1217 |
| HSV1-UL19-926 | − | GCCGGGGGCGUUUACGC | 17 | 1312 |
| HSV1-UL19-1620 | + | AGCAGGUAAAACACCGC | 17 | 2006 |
| HSV1-UL19-604 | − | GCAAGGCGGGCCACCGC | 17 | 990 |
| HSV1-UL19-805 | − | GGCCGCUGCUGCACCGC | 17 | 1191 |
| HSV1-UL19-1603 | + | GCGUCCCGGAACGCCGC | 17 | 1989 |
| HSV1-UL19-921 | − | CCCCGGGGACGCGCCGC | 17 | 1307 |
| HSV1-UL19-895 | − | ACUUUUACCUGGGCCGC | 17 | 1281 |
| HSV1-UL19-917 | − | ACGUCAACUACUUCCGC | 17 | 1303 |
| HSV1-UL19-1813 | + | CCACGACCGCGUUCCGC | 17 | 2199 |
| HSV1-UL19-1650 | + | AGGGGUGGCAGCAGCGC | 17 | 2036 |
| HSV1-UL19-780 | − | GACUGCCGGGUUAGCGC | 17 | 1166 |
| HSV1-UL19-613 | − | CGCAUGCCGACACGCGC | 17 | 999 |
| HSV1-UL19-971 | − | CGGGGAAGCCCACGCGC | 17 | 1357 |
| HSV1-UL19-912 | − | CAGGUGCCGCGGCGCGC | 17 | 1298 |
| HSV1-UL19-1755 | + | GCCGCGACAGGUCGCGC | 17 | 2141 |
| HSV1-UL19-1540 | + | GCGGGGCGGGAAGGCGC | 17 | 1926 |
| HSV1-UL19-1891 | + | GGCGGGGUCGCUGGCGC | 17 | 2277 |
| HSV1-UL19-1702 | + | GGGUGCAGGGGGUGCGC | 17 | 2088 |
| HSV1-UL19-750 | − | CGUGAACGACUACUCGC | 17 | 1136 |
| HSV1-UL19-950 | − | CCUCAACGGGGCCUCGC | 17 | 1336 |
| HSV1-UL19-1694 | + | GCUGGGGCACUCCUCGC | 17 | 2080 |
| HSV1-UL19-802 | − | GGUGCCCGCCUUCUCGC | 17 | 1188 |
| HSV1-UL19-1878 | + | CUUAAACUGUACGUCGC | 17 | 2264 |
| HSV1-UL19-1697 | + | GUCCGUCACGGGGUCGC | 17 | 2083 |
| HSV1-UL19-1892 | + | GAGGAGGGCGGGGUCGC | 17 | 2278 |
| HSV1-UL19-738 | − | CCAUCGCCGCCGUUCGC | 17 | 1124 |
| HSV1-UL19-839 | − | GUCCACGCGCUGUUCGC | 17 | 1225 |
| HSV1-UL19-603 | − | UUUUUCCUGGGCAAGGC | 17 | 989 |
| HSV1-UL19-1680 | + | CGGCCCCGCGAGAAGGC | 17 | 2066 |
| HSV1-UL19-712 | − | GGGGACGUUCCCCCGGC | 17 | 1098 |
| HSV1-UL19-682 | − | UACGUGGCGGCCCCGGC | 17 | 1068 |
| HSV1-UL19-1593 | + | AUCUCCCCCGGGCCGGC | 17 | 1979 |
| HSV1-UL19-1566 | + | UCUGCGGCGGGGCCGGC | 17 | 1952 |
| HSV1-UL19-853 | − | GUCCGCGAGAGCGCGGC | 17 | 1239 |
| HSV1-UL19-1789 | + | CCACGUUCGCGCGCGGC | 17 | 2175 |
| HSV1-UL19-845 | − | GCGCGACCUGUCGCGGC | 17 | 1231 |
| HSV1-UL19-615 | − | CGACACGCGCGGGCGGC | 17 | 1001 |
| HSV1-UL19-1569 | + | UGCUGCAUGUCUGCGGC | 17 | 1955 |
| HSV1-UL19-1675 | + | CCGCCCCCCGGUGCGGC | 17 | 2061 |
| HSV1-UL19-3227 | + | CUACCCCAAGGAAGGGC | 17 | 7826 |
| HSV1-UL19-1753 | + | GACAGGUCGCGCAGGGC | 17 | 2139 |
| HSV1-UL19-1837 | + | CCCGGGGGUUGCAGGGC | 17 | 2223 |
| HSV1-UL19-1894 | + | GCACUGCGGAGGAGGGC | 17 | 2280 |
| HSV1-UL19-691 | − | GCGCCUGGCCCACGGGC | 17 | 1077 |
| HSV1-UL19-1818 | + | CGAACACGGGGACGGGC | 17 | 2204 |
| HSV1-UL19-1594 | + | CUGGAUCUCCCCCGGGC | 17 | 1980 |
| HSV1-UL19-1644 | + | CCCGCCCAGCUCCGGGC | 17 | 2030 |
| HSV1-UL19-770 | − | CCCGGAGCUGGGCGGGC | 17 | 1156 |
| HSV1-UL19-1520 | + | UGAACAGGCCCAGGGGC | 17 | 1906 |
| HSV1-UL19-1567 | + | CAUGUCUGCGGCGGGGC | 17 | 1953 |
| HSV1-UL19-1543 | + | AAAACAGCUGGCGGGGC | 17 | 1929 |
| HSV1-UL19-1530 | + | UCCGGGUGGCCGGGGGC | 17 | 1916 |
| HSV1-UL19-907 | − | GGGAAACCGCCUGGGGC | 17 | 1293 |
| HSV1-UL19-769 | − | CCGGCCCGGAGCUGGGC | 17 | 1155 |
| HSV1-UL19-1816 | + | ACGGGGACGGGCUGGGC | 17 | 2202 |
| HSV1-UL19-1713 | + | UGCAGCGUGAGCAUGGC | 17 | 2099 |
| HSV1-UL19-858 | − | ACCUACGCGCUCAUGGC | 17 | 1244 |
| HSV1-UL19-878 | − | CCCGGCACGAAACUGGC | 17 | 1264 |
| HSV1-UL19-1546 | + | CCAAAAAAACAGCUGGC | 17 | 1932 |
| HSV1-UL19-903 | − | CGGAACGCGGUCGUGGC | 17 | 1289 |
| HSV1-UL19-1535 | + | GCGGGGAUCCGGGUGGC | 17 | 1921 |
| HSV1-UL19-1612 | + | ACGGCGGCGAUGGUGGC | 17 | 1998 |
| HSV1-UL19-1709 | + | UUGGCCACCAGAUUGGC | 17 | 2095 |
| HSV1-UL19-1732 | + | UGGUAUGGUCCAGAUGC | 17 | 2118 |
| HSV1-UL19-636 | − | CCACCUGCUGGAGAUGC | 17 | 1022 |
| HSV1-UL19-777 | − | ACCGCCAUCGCGACUGC | 17 | 1163 |
| HSV1-UL19-1486 | + | CUUUCAGGAAGGACUGC | 17 | 1872 |
| HSV1-UL19-635 | − | CGUGGGCCGCCACCUGC | 17 | 1021 |
| HSV1-UL19-704 | − | GGCCGACGUGGAGCUGC | 17 | 1090 |
| HSV1-UL19-824 | − | CGCCAUGCUCACGCUGC | 17 | 1210 |
| HSV1-UL19-742 | − | GGUGUUUUACCUGCUGC | 17 | 1128 |
| HSV1-UL19-587 | − | GAUGCUGCACGUGCUGC | 17 | 973 |
| HSV1-UL19-1500 | + | GCUCGUCCAGCGUCUGC | 17 | 1886 |
| HSV1-UL19-1688 | + | GGACCACCAUGUUCUGC | 17 | 2074 |
| HSV1-UL19-621 | − | CACCGAAGCCGACGUGC | 17 | 1007 |
| HSV1-UL19-1787 | + | CCCCGCCAGUUUCGUGC | 17 | 2173 |
| HSV1-UL19-825 | − | GCUCACGCUGCAGGUGC | 17 | 1211 |
| HSV1-UL19-1707 | + | CCAGAUUGGCCGGGUGC | 17 | 2093 |
| HSV1-UL19-1900 | + | GUGGGCUUCCCCGUUGC | 17 | 2286 |
| HSV1-UL19-1839 | + | GCGUCCCCGGGGGUUGC | 17 | 2225 |
| HSV1-UL19-815 | − | CCUGCACCCGGCCAAUC | 17 | 1201 |
| HSV1-UL19-653 | − | CCUGGGCCUGUUCAAUC | 17 | 1039 |
| HSV1-UL19-837 | − | GAUGGCCCCCCAGCAUC | 17 | 1223 |
| HSV1-UL19-1446 | + | GCAGCACGUGCAGCAUC | 17 | 1832 |
| HSV1-UL19-1656 | + | GGGCCGCGCGCCGCAUC | 17 | 2042 |
| HSV1-UL19-1577 | + | ACUGCUCCGGGGUCAUC | 17 | 1963 |
| HSV1-UL19-1873 | + | AACCCGUCUCCACGAUC | 17 | 2259 |
| HSV1-UL19-669 | − | CUCUGGAACACGCGAUC | 17 | 1055 |
| HSV1-UL19-641 | − | GCGCGUGCGCGCGGAUC | 17 | 1027 |
| HSV1-UL19-1538 | + | GGGAAGGCGCGGGGAUC | 17 | 1924 |
| HSV1-UL19-1483 | + | GGAGGCGCUGUUUGAUC | 17 | 1869 |
| HSV1-UL19-752 | − | ACGUCGUGACCUACCUC | 17 | 1138 |
| HSV1-UL19-1633 | + | ACGGCCAUGCACUCCUC | 17 | 2019 |
| HSV1-UL19-583 | − | ACGUCCAGGCCGUCCUC | 17 | 969 |
| HSV1-UL19-1646 | + | GCCUGCCCGCCCAGCUC | 17 | 2032 |
| HSV1-UL19-1850 | + | CCCGGGACGGGUCGCUC | 17 | 2236 |
| HSV1-UL19-848 | − | CCUGGUCCCCCCGGCUC | 17 | 1234 |
| HSV1-UL19-1671 | + | CGCGGCGUCCGCGGCUC | 17 | 2057 |
| HSV1-UL19-1580 | + | GGCUGCAUAAACUGCUC | 17 | 1966 |
| HSV1-UL19-1690 | + | UCGCCGGGGGCGAUCUC | 17 | 2076 |
| HSV1-UL19-667 | − | GGUGCUGCGCCUGUCUC | 17 | 1053 |
| HSV1-UL19-632 | − | CAAGGCCGUGCGAAGUC | 17 | 1018 |
| HSV1-UL19-1519 | + | GGGGCAGGACGAACGUC | 17 | 1905 |
| HSV1-UL19-1719 | + | GUGUUGGCGCCCGCGUC | 17 | 2105 |
| HSV1-UL19-1505 | + | CGCGCACGCGCGUCGUC | 17 | 1891 |
| HSV1-UL19-1643 | + | CCAGCUCCGGGCCGGUC | 17 | 2029 |
| HSV1-UL19-673 | − | ACGUUGACGCGGCGGUC | 17 | 1059 |
| HSV1-UL19-722 | − | GCCCCUGGCGCUAUGUC | 17 | 1108 |
| HSV1-UL19-1581 | + | GCCAGGUUGGCGUUGUC | 17 | 1967 |
| HSV1-UL19-1726 | + | GGCCAUCAGCAGGAUUC | 17 | 2112 |
| HSV1-UL19-1492 | + | CCAGGUUGGCCCCGUUC | 17 | 1878 |
| HSV1-UL19-724 | − | UAUGUCCGGCGGCGUUC | 17 | 1110 |
| HSV1-UL19-1499 | + | CCAGCGUCUGCCGGUUC | 17 | 1885 |
| HSV1-UL19-909 | − | AGCCCGUCCCCGUGUUC | 17 | 1295 |
| HSV1-UL19-1488 | + | CGGUGUCCUCCACUUUC | 17 | 1874 |
| HSV1-UL19-644 | − | CGAGAAGCUGGUCUUUC | 17 | 1030 |
| HSV1-UL19-965 | − | CCCGUGUGGCCUGUUUC | 17 | 1351 |
| HSV1-UL19-1782 | + | CGUUCUCAGUCACAAAG | 17 | 2168 |
| HSV1-UL19-619 | − | GCAGUCCUUCCUGAAAG | 17 | 1005 |
| HSV1-UL19-1905 | + | CGAGAUACUGCGCGAAG | 17 | 2291 |
| HSV1-UL19-1515 | + | UCCAUGGCGCCCACCAG | 17 | 1901 |
| HSV1-UL19-1758 | + | AGAGCCGGGGGGACCAG | 17 | 2144 |
| HSV1-UL19-1521 | + | GGAUUGAACAGGCCCAG | 17 | 1907 |
| HSV1-UL19-1448 | + | GGCAACAACAAAGCCAG | 17 | 1834 |
| HSV1-UL19-1600 | + | GCCGGACAUAGCGCCAG | 17 | 1986 |
| HSV1-UL19-1866 | + | UAAAGAACUUAAAGCAG | 17 | 2252 |
| HSV1-UL19-1683 | + | CCCCCGCGGUGCAGCAG | 17 | 2069 |
| HSV1-UL19-1705 | + | AGAUUGGCCGGGUGCAG | 17 | 2091 |
| HSV1-UL19-1808 | + | CGCGUUGUCCAGGAGAG | 17 | 2194 |
| HSV1-UL19-1652 | + | UCACAGUCCCACACGAG | 17 | 2038 |
| HSV1-UL19-932 | − | ACGCGGGGGACAAGGAG | 17 | 1318 |
| HSV1-UL19-1810 | + | GCCGCGUUGUCCAGGAG | 17 | 2196 |
| HSV1-UL19-1737 | + | AGCGCGUGGACGGGGAG | 17 | 2123 |
| HSV1-UL19-1890 | + | UCGCUGGCGCAGGUGAG | 17 | 2276 |
| HSV1-UL19-1908 | + | AGAGCCAGUCCCUUGAG | 17 | 2294 |
| HSV1-UL19-821 | − | CAACGGGCGCGUGGUAG | 17 | 1207 |
| HSV1-UL19-898 | − | CCCUCUCCUGGACAACG | 17 | 1284 |
| HSV1-UL19-969 | − | CCGCAGUGCCCGCAACG | 17 | 1355 |
| HSV1-UL19-949 | − | GGCCUACCACCUCAACG | 17 | 1335 |
| HSV1-UL19-883 | − | CCAGCCGCGCGCGAACG | 17 | 1269 |
| HSV1-UL19-1587 | + | CACAAAGAAGUCGAACG | 17 | 1973 |
| HSV1-UL19-900 | − | CGUGUACCUGCGGAACG | 17 | 1286 |
| HSV1-UL19-626 | − | CGAGAUGGUCCUGAACG | 17 | 1012 |
| HSV1-UL19-946 | − | GGACCUGCUCUAUAACG | 17 | 1332 |
| HSV1-UL19-786 | − | CGCGGCGGCAUGUAACG | 17 | 1172 |
| HSV1-UL19-748 | − | GCUACUGGAACAACACG | 17 | 1134 |
| HSV1-UL19-1821 | + | GGGCGCACCCGAACACG | 17 | 2207 |
| HSV1-UL19-1654 | + | CGUCACAGUCCCACACG | 17 | 2040 |
| HSV1-UL19-1832 | + | UGACGUCGGUCGACACG | 17 | 2218 |
| HSV1-UL19-1663 | + | AUGCCGCCGCGUACACG | 17 | 2049 |
| HSV1-UL19-842 | − | CGCCGGGGCCGACCACG | 17 | 1228 |
| HSV1-UL19-1746 | + | UGGGCGCGUUCGCCACG | 17 | 2132 |
| HSV1-UL19-1698 | + | GCGCGGGGUCCGUCACG | 17 | 2084 |
| HSV1-UL19-1475 | + | UCGGCAUGCGUCAGACG | 17 | 1861 |
| HSV1-UL19-633 | − | GCGAAGUCUGGACGACG | 17 | 1019 |
| HSV1-UL19-830 | − | CUCGGCGGCGCCCGACG | 17 | 1216 |
| HSV1-UL19-703 | − | UGUGGGGGUGGCCGACG | 17 | 1089 |
| HSV1-UL19-618 | − | CGGGCGGCCGGUCGACG | 17 | 1004 |
| HSV1-UL19-834 | − | CAUGCGCAUAUUCGACG | 17 | 1220 |
| HSV1-UL19-1558 | + | GAUUGGCGGCUUCGACG | 17 | 1944 |
| HSV1-UL19-1740 | + | CGAACAGCGCGUGGACG | 17 | 2126 |
| HSV1-UL19-1722 | + | CAUGUUGGUGGUGGACG | 17 | 2108 |
| HSV1-UL19-1797 | + | UGCCCAUGUCGGUGACG | 17 | 2183 |
| HSV1-UL19-670 | − | GCUGAUGAACGUUGACG | 17 | 1056 |
| HSV1-UL19-1685 | + | GCAGGGUGGCGUAUACG | 17 | 2071 |
| HSV1-UL19-678 | − | AGCCGCCAAUCCGUACG | 17 | 1064 |
| HSV1-UL19-679 | − | UCCGUACGGGGCGUACG | 17 | 1065 |
| HSV1-UL19-943 | − | GCAGCGAUUUUCGUACG | 17 | 1329 |
| HSV1-UL19-784 | − | CCACGACCCCGUGUACG | 17 | 1170 |
| HSV1-UL19-925 | − | CGCCGGGGGCGUUUACG | 17 | 1311 |
| HSV1-UL19-795 | − | GAUGACCGGCCGCACCG | 17 | 1181 |
| HSV1-UL19-804 | − | GGGCCGCUGCUGCACCG | 17 | 1190 |
| HSV1-UL19-787 | − | AUGUAACGUGGCGACCG | 17 | 1173 |
| HSV1-UL19-939 | − | CGGCCACGGCCAACCCG | 17 | 1325 |
| HSV1-UL19-1881 | + | GAGUUCGCGGCACCCCG | 17 | 2267 |
| HSV1-UL19-920 | − | CGGCCCUGCAACCCCCG | 17 | 1306 |
| HSV1-UL19-1684 | + | GUCGAAGCGAACCCCCG | 17 | 2070 |
| HSV1-UL19-961 | − | AGUUUAAGCGCCCCCCG | 17 | 1347 |
| HSV1-UL19-1631 | + | CGGGAAGGUCUCCCCCG | 17 | 2017 |
| HSV1-UL19-1806 | + | GGAGAGGGGGAGCCCCG | 17 | 2192 |
| HSV1-UL19-1444 | + | CGCGCUCAAACGCCCCG | 17 | 1830 |
| HSV1-UL19-1841 | + | CCGGCGGCGCGUCCCCG | 17 | 2227 |
| HSV1-UL19-728 | − | GCGUUCCGGGACGCCCG | 17 | 1114 |
| HSV1-UL19-1575 | + | CCCAUCGGACCCGCCCG | 17 | 1961 |
| HSV1-UL19-715 | − | UUCCCCCGGCCGGCCCG | 17 | 1101 |
| HSV1-UL19-860 | − | CUUCAAGAUCAGUCCCG | 17 | 1246 |
| HSV1-UL19-755 | − | CGGGGGAGACCUUCCCG | 17 | 1141 |
| HSV1-UL19-1763 | + | GUUGGCCCCCAGAGCCG | 17 | 2149 |
| HSV1-UL19-790 | − | CACCCAGGCCCGAGCCG | 17 | 1176 |
| HSV1-UL19-658 | − | GUUUGCCGCGCACGCCG | 17 | 1044 |
| HSV1-UL19-791 | − | CCGAGCCGCGGACGCCG | 17 | 1177 |
| HSV1-UL19-923 | − | CCGGGGACGCGCCGCCG | 17 | 1309 |
| HSV1-UL19-1826 | + | UCCAUCCCUGCGCGCCG | 17 | 2212 |
| HSV1-UL19-1692 | + | UGGGGCACUCCUCGCCG | 17 | 2078 |
| HSV1-UL19-841 | − | CCACGCGCUGUUCGCCG | 17 | 1227 |
| HSV1-UL19-1591 | + | CUCCCCCGGGCCGGCCG | 17 | 1977 |
| HSV1-UL19-1564 | + | UGCGGCGGGGCCGGCCG | 17 | 1950 |
| HSV1-UL19-855 | − | CCGCGAGAGCGCGGCCG | 17 | 1241 |
| HSV1-UL19-1751 | + | CAGGUCGCGCAGGGCCG | 17 | 2137 |
| HSV1-UL19-894 | − | AACUUUUACCUGGGCCG | 17 | 1280 |
| HSV1-UL19-1827 | + | ACACGGCGUCCUGGCCG | 17 | 2213 |
| HSV1-UL19-1849 | + | ACGGGUCGCUCUGGCCG | 17 | 2235 |
| HSV1-UL19-1469 | + | GGAGGGCUGCGUGGCCG | 17 | 1855 |
| HSV1-UL19-1874 | + | GCUGGCGGCGGUGGCCG | 17 | 2260 |
| HSV1-UL19-1533 | + | GGGGAUCCGGGUGGCCG | 17 | 1919 |
| HSV1-UL19-1856 | + | CGCCCACGGGUUGGCCG | 17 | 2242 |
| HSV1-UL19-706 | − | CCGACGUGGAGCUGCCG | 17 | 1092 |
| HSV1-UL19-911 | − | GGUGCGCCCAGGUGCCG | 17 | 1297 |
| HSV1-UL19-1794 | + | GUUGCGGACGGUUGCCG | 17 | 2180 |
| HSV1-UL19-1674 | + | GUGCGGCCGGUCAUCCG | 17 | 2060 |
| HSV1-UL19-611 | − | GGCCACGCAGCCCUCCG | 17 | 997 |
| HSV1-UL19-1578 | + | CUGCAUAAACUGCUCCG | 17 | 1964 |
| HSV1-UL19-1666 | + | GUUGCGGUUGAAGUCCG | 17 | 2052 |
| HSV1-UL19-1673 | + | GUCAUCCGCGGCGUCCG | 17 | 2059 |
| HSV1-UL19-852 | − | GCACGUCCGCGAGAGCG | 17 | 1238 |
| HSV1-UL19-1906 | + | AGCCAGUCCCUUGAGCG | 17 | 2292 |
| HSV1-UL19-585 | − | CUCGGGGCGUUUGAGCG | 17 | 971 |
| HSV1-UL19-779 | − | CGACUGCCGGGUUAGCG | 17 | 1165 |
| HSV1-UL19-1585 | + | CAAAGAAGUCGAACGCG | 17 | 1971 |
| HSV1-UL19-612 | − | ACGCAUGCCGACACGCG | 17 | 998 |
| HSV1-UL19-970 | − | ACGGGGAAGCCCACGCG | 17 | 1356 |
| HSV1-UL19-927 | − | CCGGGGGCGUUUACGCG | 17 | 1313 |
| HSV1-UL19-806 | − | GCCGCUGCUGCACCGCG | 17 | 1192 |
| HSV1-UL19-896 | − | CUUUUACCUGGGCCGCG | 17 | 1282 |
| HSV1-UL19-1743 | + | CCCCGGCGAACAGCGCG | 17 | 2129 |
| HSV1-UL19-781 | − | ACUGCCGGGUUAGCGCG | 17 | 1167 |
| HSV1-UL19-1903 | + | AGUGGGUCUCCCGCGCG | 17 | 2289 |
| HSV1-UL19-1790 | + | AGGUCCACGUUCGCGCG | 17 | 2176 |
| HSV1-UL19-1539 | + | CGGGGCGGGAAGGCGCG | 17 | 1925 |
| HSV1-UL19-775 | − | CGCCCUGAUGCGGCGCG | 17 | 1161 |
| HSV1-UL19-820 | − | GUUUCACAACGGGCGCG | 17 | 1206 |
| HSV1-UL19-719 | − | CCAGGCCACCUGGCGCG | 17 | 1105 |
| HSV1-UL19-640 | − | GACGACGCGCGUGCGCG | 17 | 1026 |
| HSV1-UL19-1701 | + | GGUGCAGGGGGUGCGCG | 17 | 2087 |
| HSV1-UL19-803 | − | GUGCCCGCCUUCUCGCG | 17 | 1189 |
| HSV1-UL19-739 | − | CAUCGCCGCCGUUCGCG | 17 | 1125 |
| HSV1-UL19-1541 | + | GGCGGGGCGGGAAGGCG | 17 | 1927 |
| HSV1-UL19-1864 | + | GGGGCUGAGCACCGGCG | 17 | 2250 |
| HSV1-UL19-812 | − | GGAGAUCGCCCCCGGCG | 17 | 1198 |
| HSV1-UL19-1568 | + | GCUGCAUGUCUGCGGCG | 17 | 1954 |
| HSV1-UL19-3228 | + | UACCCCAAGGAAGGGCG | 17 | 7827 |
| HSV1-UL19-1893 | + | CACUGCGGAGGAGGGCG | 17 | 2279 |
| HSV1-UL19-1529 | + | CCGGGUGGCCGGGGGCG | 17 | 1915 |
| HSV1-UL19-886 | − | GAACGUGGACCUGGGCG | 17 | 1272 |
| HSV1-UL19-1608 | + | UGGCGGGGGCCAUGGCG | 17 | 1994 |
| HSV1-UL19-859 | − | CCUACGCGCUCAUGGCG | 17 | 1245 |
| HSV1-UL19-879 | − | CCGGCACGAAACUGGCG | 17 | 1265 |
| HSV1-UL19-1545 | + | CAAAAAAACAGCUGGCG | 17 | 1931 |
| HSV1-UL19-1611 | + | CGGCGGCGAUGGUGGCG | 17 | 1997 |
| HSV1-UL19-1470 | + | GGCCACGGAGGGCUGCG | 17 | 1856 |
| HSV1-UL19-1526 | + | UAGGUCCCCGGCGUGCG | 17 | 1912 |
| HSV1-UL19-749 | − | GAACAACACGCGGUGCG | 17 | 1135 |
| HSV1-UL19-1703 | + | CGGGUGCAGGGGGUGCG | 17 | 2089 |
| HSV1-UL19-953 | − | CCUGGAGCGCCUGAUCG | 17 | 1339 |
| HSV1-UL19-1482 | + | GAGGCGCUGUUUGAUCG | 17 | 1868 |
| HSV1-UL19-962 | − | GGGGUGCCGCGAACUCG | 17 | 1348 |
| HSV1-UL19-753 | − | CGUCGUGACCUACCUCG | 17 | 1139 |
| HSV1-UL19-584 | − | CGUCCAGGCCGUCCUCG | 17 | 970 |
| HSV1-UL19-746 | − | CGCCCUGGCCCGGCUCG | 17 | 1132 |
| HSV1-UL19-609 | − | CGUCGAGGCCUGGCUCG | 17 | 995 |
| HSV1-UL19-1770 | + | CCCCGGCCGCGCUCUCG | 17 | 2156 |
| HSV1-UL19-801 | − | UGGUGCCCGCCUUCUCG | 17 | 1187 |
| HSV1-UL19-761 | − | CCUGGUGGCGCACGUCG | 17 | 1147 |
| HSV1-UL19-888 | − | GGGCUACACCGCCGUCG | 17 | 1274 |
| HSV1-UL19-607 | − | CCGCCGGGAGGCCGUCG | 17 | 993 |
| HSV1-UL19-901 | − | CCUGCGGAACGCGGUCG | 17 | 1287 |
| HSV1-UL19-674 | − | CGUUGACGCGGCGGUCG | 17 | 1060 |
| HSV1-UL19-1662 | + | CCGCGUACACGGGGUCG | 17 | 2048 |
| HSV1-UL19-1557 | + | CGGCUUCGACGGGGUCG | 17 | 1943 |
| HSV1-UL19-694 | − | GCGGGUCCGAUGGGUCG | 17 | 1080 |
| HSV1-UL19-844 | − | CCCUGCGCGACCUGUCG | 17 | 1230 |
| HSV1-UL19-937 | − | CCCGUCCCGGGCCUUCG | 17 | 1323 |
| HSV1-UL19-1884 | + | GGUCCUCCACGAGUUCG | 17 | 2270 |
| HSV1-UL19-737 | − | ACCAUCGCCGCCGUUCG | 17 | 1123 |
| HSV1-UL19-930 | − | UUACGCGGGGGACAAGG | 17 | 1316 |
| HSV1-UL19-602 | − | CUUUUUCCUGGGCAAGG | 17 | 988 |
| HSV1-UL19-1681 | + | GCGGCCCCGCGAGAAGG | 17 | 2067 |
| HSV1-UL19-1514 | + | CCAUGGCGCCCACCAGG | 17 | 1900 |
| HSV1-UL19-1757 | + | GAGCCGGGGGGACCAGG | 17 | 2143 |
| HSV1-UL19-1814 | + | GCUGGGCCGGCCCCAGG | 17 | 2200 |
| HSV1-UL19-1598 | + | GUUCACCACGCGCCAGG | 17 | 1984 |
| HSV1-UL19-1453 | + | CCACCCUGGUGGCCAGG | 17 | 1839 |
| HSV1-UL19-874 | − | CCUGGGCCAGCUCCAGG | 17 | 1260 |
| HSV1-UL19-1497 | + | CCUGCAUCUCCAGCAGG | 17 | 1883 |
| HSV1-UL19-637 | − | CCUGCUGGAGAUGCAGG | 17 | 1023 |
| HSV1-UL19-1485 | + | UCAGGAAGGACUGCAGG | 17 | 1871 |
| HSV1-UL19-1704 | + | GAUUGGCCGGGUGCAGG | 17 | 2090 |
| HSV1-UL19-966 | − | GUGUGGCCUGUUUCAGG | 17 | 1352 |
| HSV1-UL19-1807 | + | GCGUUGUCCAGGAGAGG | 17 | 2193 |
| HSV1-UL19-933 | − | CGCGGGGGACAAGGAGG | 17 | 1319 |
| HSV1-UL19-1897 | + | UGCGGGCACUGCGGAGG | 17 | 2283 |
| HSV1-UL19-1736 | + | GCGCGUGGACGGGGAGG | 17 | 2122 |
| HSV1-UL19-1862 | + | GCGAGGCCCCGUUGAGG | 17 | 2248 |
| HSV1-UL19-1616 | + | GAACGCCCCGCGAACGG | 17 | 2002 |
| HSV1-UL19-1831 | + | GACGUCGGUCGACACGG | 17 | 2217 |
| HSV1-UL19-1472 | + | ACGGGGCACGGCCACGG | 17 | 1858 |
| HSV1-UL19-1792 | + | UGCCGUGGCCACGACGG | 17 | 2178 |
| HSV1-UL19-796 | − | AUGACCGGCCGCACCGG | 17 | 1182 |
| HSV1-UL19-741 | − | CGACCGCAACUACCCGG | 17 | 1127 |
| HSV1-UL19-1880 | + | AGUUCGCGGCACCCCGG | 17 | 2266 |
| HSV1-UL19-1844 | + | CGCGUAAACGCCCCCGG | 17 | 2230 |
| HSV1-UL19-1840 | + | CGGCGGCGCGUCCCCGG | 17 | 2226 |
| HSV1-UL19-716 | − | UCCCCCGGCCGGCCCGG | 17 | 1102 |
| HSV1-UL19-1464 | + | CCUCGACGGCCUCCCGG | 17 | 1850 |
| HSV1-UL19-1762 | + | UUGGCCCCCAGAGCCGG | 17 | 2148 |
| HSV1-UL19-924 | − | CGGGGACGCGCCGCCGG | 17 | 1310 |
| HSV1-UL19-1691 | + | GGGGCACUCCUCGCCGG | 17 | 2077 |
| HSV1-UL19-1590 | + | UCCCCCGGGCCGGCCGG | 17 | 1976 |
| HSV1-UL19-1750 | + | AGGUCGCGCAGGGCCGG | 17 | 2136 |
| HSV1-UL19-1836 | + | GGGGGUUGCAGGGCCGG | 17 | 2222 |
| HSV1-UL19-1532 | + | GGGAUCCGGGUGGCCGG | 17 | 1918 |
| HSV1-UL19-707 | − | CGACGUGGAGCUGCCGG | 17 | 1093 |
| HSV1-UL19-723 | − | CCUGGCGCUAUGUCCGG | 17 | 1109 |
| HSV1-UL19-671 | − | GAUGAACGUUGACGCGG | 17 | 1057 |
| HSV1-UL19-785 | − | CGACCCCGUGUACGCGG | 17 | 1171 |
| HSV1-UL19-928 | − | CGGGGGCGUUUACGCGG | 17 | 1314 |
| HSV1-UL19-807 | − | CCGCUGCUGCACCGCGG | 17 | 1193 |
| HSV1-UL19-782 | − | CUGCCGGGUUAGCGCGG | 17 | 1168 |
| HSV1-UL19-1463 | + | CGACGGCCUCCCGGCGG | 17 | 1849 |
| HSV1-UL19-1875 | + | UACGUCGCUGGCGGCGG | 17 | 2261 |
| HSV1-UL19-1480 | + | CUGUUUGAUCGGGGCGG | 17 | 1866 |
| HSV1-UL19-880 | − | CGGCACGAAACUGGCGG | 17 | 1266 |
| HSV1-UL19-1876 | + | CUGUACGUCGCUGGCGG | 17 | 2262 |
| HSV1-UL19-1610 | + | GGCGGCGAUGGUGGCGG | 17 | 1996 |
| HSV1-UL19-1898 | + | CGUUGCGGGCACUGCGG | 17 | 2284 |
| HSV1-UL19-1570 | + | CUGCUGCAUGUCUGCGG | 17 | 1956 |
| HSV1-UL19-754 | − | GUCGUGACCUACCUCGG | 17 | 1140 |
| HSV1-UL19-829 | − | GGCGCUGCUCUGCUCGG | 17 | 1215 |
| HSV1-UL19-870 | − | CUCGGAGCGCGCGUCGG | 17 | 1256 |
| HSV1-UL19-675 | − | GUUGACGCGGCGGUCGG | 17 | 1061 |
| HSV1-UL19-957 | − | CGUGGAGACGGGUUCGG | 17 | 1343 |
| HSV1-UL19-1553 | + | CGUUCAUCAGCGAAGGG | 17 | 1939 |
| HSV1-UL19-3229 | + | ACUACCCCAAGGAAGGG | 17 | 7828 |
| HSV1-UL19-595 | − | CCGCCUGGCCACCAGGG | 17 | 981 |
| HSV1-UL19-1686 | + | CACCAUGUUCUGCAGGG | 17 | 2072 |
| HSV1-UL19-1895 | + | GGCACUGCGGAGGAGGG | 17 | 2281 |
| HSV1-UL19-1846 | + | GUGGUCGUACAUGAGGG | 17 | 2232 |
| HSV1-UL19-690 | − | AGCGCCUGGCCCACGGG | 17 | 1076 |
| HSV1-UL19-797 | − | UGACCGGCCGCACCGGG | 17 | 1183 |
| HSV1-UL19-1879 | + | GUUCGCGGCACCCCGGG | 17 | 2265 |
| HSV1-UL19-1761 | + | UGGCCCCCAGAGCCGGG | 17 | 2147 |
| HSV1-UL19-606 | − | GGCGGGCCACCGCCGGG | 17 | 992 |
| HSV1-UL19-1749 | + | GGUCGCGCAGGGCCGGG | 17 | 2135 |
| HSV1-UL19-708 | − | GACGUGGAGCUGCCGGG | 17 | 1094 |
| HSV1-UL19-1536 | + | AGGCGCGGGGAUCCGGG | 17 | 1922 |
| HSV1-UL19-783 | − | UGCCGGGUUAGCGCGGG | 17 | 1169 |
| HSV1-UL19-614 | − | AUGCCGACACGCGCGGG | 17 | 1000 |
| HSV1-UL19-881 | − | GGCACGAAACUGGCGGG | 17 | 1267 |
| HSV1-UL19-1447 | + | AACAACAAAGCCAGGGG | 17 | 1833 |
| HSV1-UL19-1651 | + | CAGUCCCACACGAGGGG | 17 | 2037 |
| HSV1-UL19-1739 | + | ACAGCGCGUGGACGGGG | 17 | 2125 |
| HSV1-UL19-709 | − | ACGUGGAGCUGCCGGGG | 17 | 1095 |
| HSV1-UL19-3230 | + | CCCAAGGAAGGGCGGGG | 17 | 7829 |
| HSV1-UL19-882 | − | GCACGAAACUGGCGGGG | 17 | 1268 |
| HSV1-UL19-1544 | + | AAAAACAGCUGGCGGGG | 17 | 1930 |
| HSV1-UL19-1481 | + | GCGCUGUUUGAUCGGGG | 17 | 1867 |
| HSV1-UL19-1830 | + | GUCGGUCGACACGGGGG | 17 | 2216 |
| HSV1-UL19-1531 | + | AUCCGGGUGGCCGGGGG | 17 | 1917 |
| HSV1-UL19-710 | − | CGUGGAGCUGCCGGGGG | 17 | 1096 |
| HSV1-UL19-798 | − | CCGGCCGCACCGGGGGG | 17 | 1184 |
| HSV1-UL19-734 | − | GGGCCUGGAGCUGGGGG | 17 | 1120 |
| HSV1-UL19-702 | − | CGACUUCUUUGUGGGGG | 17 | 1088 |
| HSV1-UL19-768 | − | ACCGGCCCGGAGCUGGG | 17 | 1154 |
| HSV1-UL19-1728 | + | AUGGUCCAGAUGCUGGG | 17 | 2114 |
| HSV1-UL19-1501 | + | CACGCGCGUCGUCUGGG | 17 | 1887 |
| HSV1-UL19-809 | − | CACCCUGCAGAACAUGG | 17 | 1195 |
| HSV1-UL19-1714 | + | CUGCAGCGUGAGCAUGG | 17 | 2100 |
| HSV1-UL19-857 | − | GACCUACGCGCUCAUGG | 17 | 1243 |
| HSV1-UL19-1659 | + | CCCGGCAGUCGCGAUGG | 17 | 2045 |
| HSV1-UL19-1614 | + | GCGAACGGCGGCGAUGG | 17 | 2000 |
| HSV1-UL19-1776 | + | ACCCAAACCCGGGAUGG | 17 | 2162 |
| HSV1-UL19-1622 | + | CUCGCUGCCGUGUAUGG | 17 | 2008 |
| HSV1-UL19-877 | − | GCCCGGCACGAAACUGG | 17 | 1263 |
| HSV1-UL19-1549 | + | ACAGGCGCAGCACCUGG | 17 | 1935 |
| HSV1-UL19-760 | − | CGUGUACCGGGACCUGG | 17 | 1146 |
| HSV1-UL19-1455 | + | CGCCCGGGCCACCCUGG | 17 | 1841 |
| HSV1-UL19-648 | − | CGUUCCCUACCCCCUGG | 17 | 1034 |
| HSV1-UL19-686 | − | UGUUUUUGAACGCCUGG | 17 | 1072 |
| HSV1-UL19-1547 | + | CCCAAAAAAACAGCUGG | 17 | 1933 |
| HSV1-UL19-733 | − | CCGGGGCCUGGAGCUGG | 17 | 1119 |
| HSV1-UL19-1877 | + | AAACUGUACGUCGCUGG | 17 | 2263 |
| HSV1-UL19-1729 | + | UAUGGUCCAGAUGCUGG | 17 | 2115 |
| HSV1-UL19-816 | − | GCACCCGGCCAAUCUGG | 17 | 1202 |
| HSV1-UL19-851 | − | GGUCCCCCCGGCUCUGG | 17 | 1237 |
| HSV1-UL19-1502 | + | GCACGCGCGUCGUCUGG | 17 | 1888 |
| HSV1-UL19-645 | − | GAAGCUGGUCUUUCUGG | 17 | 1031 |
| HSV1-UL19-620 | − | GUCCUUCCUGAAAGUGG | 17 | 1006 |
| HSV1-UL19-680 | − | GUACGGGGCGUACGUGG | 17 | 1066 |
| HSV1-UL19-963 | − | GUGCCGCGAACUCGUGG | 17 | 1349 |
| HSV1-UL19-902 | − | GCGGAACGCGGUCGUGG | 17 | 1288 |
| HSV1-UL19-1496 | + | GCAUCUCCAGCAGGUGG | 17 | 1882 |
| HSV1-UL19-1613 | + | AACGGCGGCGAUGGUGG | 17 | 1999 |
| HSV1-UL19-1509 | + | GUAGGGAACGUUGGUGG | 17 | 1895 |
| HSV1-UL19-1723 | + | UAUGCGCAUGUUGGUGG | 17 | 2109 |
| HSV1-UL19-701 | − | GUUCGACUUCUUUGUGG | 17 | 1087 |
| HSV1-UL19-1561 | + | CGCCCCGUACGGAUUGG | 17 | 1947 |
| HSV1-UL19-1510 | + | GGGGUAGGGAACGUUGG | 17 | 1896 |
| HSV1-UL19-1724 | + | GAAUAUGCGCAUGUUGG | 17 | 2110 |
| HSV1-UL19-1870 | + | CAGGCAGCGAUGUUUGG | 17 | 2256 |
| HSV1-UL19-664 | − | GCCAGCUGUUUUUUUGG | 17 | 1050 |
| HSV1-UL19-1848 | + | GGCCGUGGUCGUACAUG | 17 | 2234 |
| HSV1-UL19-774 | − | ACUGUGACGCCCUGAUG | 17 | 1160 |
| HSV1-UL19-1899 | + | CCCCGUUGCGGGCACUG | 17 | 2285 |
| HSV1-UL19-899 | − | CGGCAGCCGUGUACCUG | 17 | 1285 |
| HSV1-UL19-685 | − | CUGUUUUUGAACGCCUG | 17 | 1071 |
| HSV1-UL19-906 | − | UGGCGGGAAACCGCCUG | 17 | 1292 |
| HSV1-UL19-732 | − | CCCGGGGCCUGGAGCUG | 17 | 1118 |
| HSV1-UL19-1695 | + | CCGUCACGGGGUCGCUG | 17 | 2081 |
| HSV1-UL19-1730 | + | GUAUGGUCCAGAUGCUG | 17 | 2116 |
| HSV1-UL19-850 | − | UGGUCCCCCCGGCUCUG | 17 | 1236 |
| HSV1-UL19-1503 | + | CGCACGCGCGUCGUCUG | 17 | 1889 |
| HSV1-UL19-1571 | + | CAGCUGCUGCAUGUCUG | 17 | 1957 |
| HSV1-UL19-964 | − | CUCGUGGAGGACCCGUG | 17 | 1350 |
| HSV1-UL19-772 | − | UGCUGCCACCCCUCGUG | 17 | 1158 |
| HSV1-UL19-1676 | + | CAGUCCGCCCCCCGGUG | 17 | 2062 |
| HSV1-UL19-1468 | + | GCUGCGUGGCCGUGGUG | 17 | 1854 |
| HSV1-UL19-700 | − | CGUUCGACUUCUUUGUG | 17 | 1086 |
| HSV1-UL19-3231 | − | CGCCCCGCCCUUCCUUG | 17 | 7830 |
| HSV1-UL19-1679 | + | CGUAGUAGUAAAUCUUG | 17 | 2065 |
| HSV1-UL19-1618 | + | ACACCGCCGGGUAGUUG | 17 | 2004 |
| HSV1-UL19-1863 | + | CCGGCGAGGCCCCGUUG | 17 | 2249 |
| HSV1-UL19-1901 | + | CGUGGGCUUCCCCGUUG | 17 | 2287 |
| HSV1-UL19-1667 | + | GCAGCUGGCCGUCGUUG | 17 | 2053 |
| HSV1-UL19-1796 | + | UGUCGGUGACGGGGUUG | 17 | 2182 |
| HSV1-UL19-1717 | + | UGCGUUCGGCCAUGUUG | 17 | 2103 |
| HSV1-UL19-698 | − | CGCGUUCGACUUCUUUG | 17 | 1084 |
| HSV1-UL19-1802 | + | CCCAGGUAAAAGUUUUG | 17 | 2188 |
| HSV1-UL19-663 | − | CGCCAGCUGUUUUUUUG | 17 | 1049 |
| HSV1-UL19-1748 | + | CAGGGCCGGGGGGAAAU | 17 | 2134 |
| HSV1-UL19-891 | − | AACCCCGUCACCGACAU | 17 | 1277 |
| HSV1-UL19-1576 | + | GGCCUUCCGCGACCCAU | 17 | 1962 |
| HSV1-UL19-642 | − | GCGGAUCUGGUGUCCAU | 17 | 1028 |
| HSV1-UL19-1451 | + | UCCAGGUAUCGUUGCAU | 17 | 1837 |
| HSV1-UL19-1710 | + | CGUGUUGGCCACCAGAU | 17 | 2096 |
| HSV1-UL19-693 | − | CCACGGGCGGGUCCGAU | 17 | 1079 |
| HSV1-UL19-668 | − | UCUCUGGAACACGCGAU | 17 | 1054 |
| HSV1-UL19-1562 | + | GUACGCCCCGUACGGAU | 17 | 1948 |
| HSV1-UL19-630 | − | GUCACGGCGCUCGUGAU | 17 | 1016 |
| HSV1-UL19-1484 | + | AGGAGGCGCUGUUUGAU | 17 | 1870 |
| HSV1-UL19-1528 | + | GGCCGGGGGCGGGGACU | 17 | 1914 |
| HSV1-UL19-1824 | + | UGCGCGCCGCGGCACCU | 17 | 2210 |
| HSV1-UL19-885 | − | CGCGCGAACGUGGACCU | 17 | 1271 |
| HSV1-UL19-751 | − | UACGUCGUGACCUACCU | 17 | 1137 |
| HSV1-UL19-893 | − | CCCCAAAACUUUUACCU | 17 | 1279 |
| HSV1-UL19-652 | − | ACGUUCGUCCUGCCCCU | 17 | 1038 |
| HSV1-UL19-684 | − | GCUGUUUUUGAACGCCU | 17 | 1070 |
| HSV1-UL19-905 | − | GUGGCGGGAAACCGCCU | 17 | 1291 |
| HSV1-UL19-1669 | + | GUCCGCGGCUCGGGCCU | 17 | 2055 |
| HSV1-UL19-1634 | + | CACGGCCAUGCACUCCU | 17 | 2020 |
| HSV1-UL19-582 | − | AACGUCCAGGCCGUCCU | 17 | 968 |
| HSV1-UL19-872 | − | UCGGAGGCGUACUUCCU | 17 | 1258 |
| HSV1-UL19-3232 | − | ACCGCCCCGCCCUUCCU | 17 | 7831 |
| HSV1-UL19-600 | − | GAGACGAGCUUUUUCCU | 17 | 986 |
| HSV1-UL19-1640 | + | AAAGUCAUCAACCAGCU | 17 | 2026 |
| HSV1-UL19-1647 | + | CAUUAGGUGAUUCAGCU | 17 | 2033 |
| HSV1-UL19-767 | − | CUGACCGGCCCGGAGCU | 17 | 1153 |
| HSV1-UL19-731 | − | GCCCGGGGCCUGGAGCU | 17 | 1117 |
| HSV1-UL19-1461 | + | GAAGCUUCGCUUUAGCU | 17 | 1847 |
| HSV1-UL19-1696 | + | UCCGUCACGGGGUCGCU | 17 | 2082 |
| HSV1-UL19-1672 | + | CCGCGGCGUCCGCGGCU | 17 | 2058 |
| HSV1-UL19-1788 | + | CACGUUCGCGCGCGGCU | 17 | 2174 |
| HSV1-UL19-1817 | + | GAACACGGGGACGGGCU | 17 | 2203 |
| HSV1-UL19-1731 | + | GGUAUGGUCCAGAUGCU | 17 | 2117 |
| HSV1-UL19-828 | − | AACGGCGCUGCUCUGCU | 17 | 1214 |
| HSV1-UL19-849 | − | CUGGUCCCCCCGGCUCU | 17 | 1235 |
| HSV1-UL19-1504 | + | GCGCACGCGCGUCGUCU | 17 | 1890 |
| HSV1-UL19-868 | − | UGAGAACGUGCUGUUCU | 17 | 1254 |
| HSV1-UL19-1904 | + | GAGAUACUGCGCGAAGU | 17 | 2290 |
| HSV1-UL19-1766 | + | GAUCGACGAAAAGUAGU | 17 | 2152 |
| HSV1-UL19-1511 | + | CAGGGGGUAGGGAACGU | 17 | 1897 |
| HSV1-UL19-1588 | + | CCCCGGCAGCUCCACGU | 17 | 1974 |
| HSV1-UL19-1490 | + | GUACGUCACCGGCACGU | 17 | 1876 |
| HSV1-UL19-634 | − | CGAAGUCUGGACGACGU | 17 | 1020 |
| HSV1-UL19-1835 | + | GCGGAAGUAGUUGACGU | 17 | 2221 |
| HSV1-UL19-951 | − | CUUUAAGUUCUUUACGU | 17 | 1337 |
| HSV1-UL19-940 | − | GGCCACGGCCAACCCGU | 17 | 1326 |
| HSV1-UL19-1574 | + | CCAUCGGACCCGCCCGU | 17 | 1960 |
| HSV1-UL19-1720 | + | GGUGUUGGCGCCCGCGU | 17 | 2106 |
| HSV1-UL19-1772 | + | CCCCGCCAUGAGCGCGU | 17 | 2158 |
| HSV1-UL19-1902 | + | GUGGGUCUCCCGCGCGU | 17 | 2288 |
| HSV1-UL19-869 | − | GUUCUCGGAGCGCGCGU | 17 | 1255 |
| HSV1-UL19-887 | − | AACGUGGACCUGGGCGU | 17 | 1273 |
| HSV1-UL19-1493 | + | GAGCGCCGUGACCAGGU | 17 | 1879 |
| HSV1-UL19-1583 | + | CAGCUCAAGAGCCAGGU | 17 | 1969 |
| HSV1-UL19-1630 | + | AAGGUCUCCCCCGAGGU | 17 | 2016 |
| HSV1-UL19-672 | − | AACGUUGACGCGGCGGU | 17 | 1058 |
| HSV1-UL19-1857 | + | CUGCGACGCCCACGGGU | 17 | 2243 |
| HSV1-UL19-1888 | + | GGCGCAGGUGAGCGGGU | 17 | 2274 |
| HSV1-UL19-1513 | + | GGCGCCCACCAGGGGGU | 17 | 1899 |
| HSV1-UL19-735 | − | GGCCUGGAGCUGGGGGU | 17 | 1121 |
| HSV1-UL19-649 | − | GUUCCCUACCCCCUGGU | 17 | 1035 |
| HSV1-UL19-1445 | + | CACGUGCAGCAUCUGGU | 17 | 1831 |
| HSV1-UL19-1745 | + | CGCGUUCGCCACGUGGU | 17 | 2131 |
| HSV1-UL19-1861 | + | GGCCCCGUUGAGGUGGU | 17 | 2247 |
| HSV1-UL19-1800 | + | GGGAAGGUUGCCCAUGU | 17 | 2186 |
| HSV1-UL19-1725 | + | GUCGAAUAUGCGCAUGU | 17 | 2111 |
| HSV1-UL19-1869 | + | AUGUUUGGCGGCGAUGU | 17 | 2255 |
| HSV1-UL19-1781 | + | UCACAAAGCGGUCCUGU | 17 | 2167 |
| HSV1-UL19-3215 | + | CGUUGCGAGCGAGCUGU | 17 | 7814 |
| HSV1-UL19-1767 | + | GCUGCACGACGGGCUGU | 17 | 2153 |
| HSV1-UL19-1711 | + | CAUGGCGUUGACCGUGU | 17 | 2097 |
| HSV1-UL19-1478 | + | CGGCCGCCCGCGCGUGU | 17 | 1864 |
| HSV1-UL19-773 | − | GCUGCCACCCCUCGUGU | 17 | 1159 |
| HSV1-UL19-1721 | + | GGUGGUGGACGCGGUGU | 17 | 2107 |
| HSV1-UL19-1582 | + | AGCCAGGUUGGCGUUGU | 17 | 1968 |
| HSV1-UL19-1716 | + | GCGUUCGGCCAUGUUGU | 17 | 2102 |
| HSV1-UL19-699 | − | GCGUUCGACUUCUUUGU | 17 | 1085 |
| HSV1-UL19-1747 | + | AGGGCCGGGGGGAAAUU | 17 | 2133 |
| HSV1-UL19-1648 | + | GCGCCGGGUCUCGCAUU | 17 | 2034 |
| HSV1-UL19-3233 | − | CCGCCCCGCCCUUCCUU | 17 | 7832 |
| HSV1-UL19-1489 | + | CACCGGCACGUCGGCUU | 17 | 1875 |
| HSV1-UL19-1718 | + | CAGCGCCGUUGUGCGUU | 17 | 2104 |
| HSV1-UL19-956 | − | GAUCGUGGAGACGGGUU | 17 | 1342 |
| HSV1-UL19-865 | − | GGCCUCCAUCCCGGGUU | 17 | 1251 |
| HSV1-UL19-1871 | + | CUCCAGGCAGCGAUGUU | 17 | 2257 |
| HSV1-UL19-908 | − | CAGCCCGUCCCCGUGUU | 17 | 1294 |
| HSV1-UL19-1735 | + | AAAAAUAGUCGCCAUUU | 17 | 2121 |
| HSV1-UL19-1804 | + | GGCCCAGGUAAAAGUUU | 17 | 2190 |
| HSV1-UL19-866 | − | GCCUCCAUCCCGGGUUU | 17 | 1252 |
| HSV1-UL19-1803 | + | GCCCAGGUAAAAGUUUU | 17 | 2189 |
| HSV1-UL19-661 | − | CCCGCCAGCUGUUUUUU | 17 | 1047 |
| HSV1-UL19-662 | − | CCGCCAGCUGUUUUUUU | 17 | 1048 |
| HSV1-UL19-445 | − | CUGGACCAUACCAUCCAAAA | 20 | 831 |
| HSV1-UL19-195 | − | GCUGCACGUGCUGCUGGAAA | 20 | 581 |
| HSV1-UL19-434 | − | CAACAUGGCCGAACGCACAA | 20 | 820 |
| HSV1-UL19-425 | − | GUCAACGCCAUGUUUCACAA | 20 | 811 |
| HSV1-UL19-198 | − | AUGCAACGAUACCUGGACAA | 20 | 584 |
| HSV1-UL19-574 | − | CUCCUCCGCAGUGCCCGCAA | 20 | 960 |
| HSV1-UL19-554 | − | AACGGGGCCUACCACCUCAA | 20 | 940 |
| HSV1-UL19-580 | − | UAUGACGCAUCCCCGCUCAA | 20 | 966 |
| HSV1-UL19-1087 | + | CGUCAACGUUCAUCAGCGAA | 20 | 1473 |
| HSV1-UL19-1150 | + | GUCGUCGAACGCCCCGCGAA | 20 | 1536 |
| HSV1-UL19-262 | − | UGUUCAAUCCGGUCAUGGAA | 20 | 648 |
| HSV1-UL19-231 | − | UACGGCGAGAUGGUCCUGAA | 20 | 617 |
| HSV1-UL19-327 | − | GCCACCUGGCGCGUGGUGAA | 20 | 713 |
| HSV1-UL19-551 | − | UACGGGGACCUGCUCUAUAA | 20 | 937 |
| HSV1-UL19-1245 | + | CACGCGCCCGUUGUGAAACA | 20 | 1631 |
| HSV1-UL19-433 | − | GCAGGUGCUGGCCCACAACA | 20 | 819 |
| HSV1-UL19-424 | − | GGCCAAUCUGGUGGCCAACA | 20 | 810 |
| HSV1-UL19-415 | − | AUACGCCACCCUGCAGAACA | 20 | 801 |
| HSV1-UL19-1356 | + | CACCUGGGCGCACCCGAACA | 20 | 1742 |
| HSV1-UL19-1362 | + | GGUGGCGAUAAACUCACACA | 20 | 1748 |
| HSV1-UL19-1419 | + | GCCUCCUGAAACAGGCCACA | 20 | 1805 |
| HSV1-UL19-1085 | + | CAUCAGCGAAGGGUGGCACA | 20 | 1471 |
| HSV1-UL19-497 | − | CCGCAACCCCGUCACCGACA | 20 | 883 |
| HSV1-UL19-1367 | + | GUAGUUGACGUCGGUCGACA | 20 | 1753 |
| HSV1-UL19-536 | − | GGGCGUUUACGCGGGGGACA | 20 | 922 |
| HSV1-UL19-350 | − | CUGCUGCAGGCCGCCAUACA | 20 | 736 |
| HSV1-UL19-1198 | + | GUUACAUGCCGCCGCGUACA | 20 | 1584 |
| HSV1-UL19-1345 | + | GACCGCGUUCCGCAGGUACA | 20 | 1731 |
| HSV1-UL19-1168 | + | CGCCACCAGGUCCCGGUACA | 20 | 1554 |
| HSV1-UL19-1049 | + | CAGGUCCAUGGCGCCCACCA | 20 | 1435 |
| HSV1-UL19-201 | − | CAACGGCCGCCUGGCCACCA | 20 | 587 |
| HSV1-UL19-217 | − | CUGGCUCGUGGACCUCACCA | 20 | 603 |
| HSV1-UL19-541 | − | ACCGCCCUCAUGUACGACCA | 20 | 927 |
| HSV1-UL19-992 | + | UCGCUUUAGCUCGGCGACCA | 20 | 1378 |
| HSV1-UL19-1292 | + | CCCCAGAGCCGGGGGGACCA | 20 | 1678 |
| HSV1-UL19-522 | − | CGGCGCGCAGGGAUGGACCA | 20 | 908 |
| HSV1-UL19-1392 | + | GAAAAUCGCUGCGACGCCCA | 20 | 1778 |
| HSV1-UL19-1055 | + | GACCGGAUUGAACAGGCCCA | 20 | 1441 |
| HSV1-UL19-295 | − | UGGGGGCAGCGCCUGGCCCA | 20 | 681 |
| HSV1-UL19-982 | + | CAUCGGCAACAACAAAGCCA | 20 | 1368 |
| HSV1-UL19-343 | − | GGGGGUGGGACGCCACGCCA | 20 | 729 |
| HSV1-UL19-1134 | + | CGCCGCCGGACAUAGCGCCA | 20 | 1520 |
| HSV1-UL19-257 | − | CUACCCCCUGGUGGGCGCCA | 20 | 643 |
| HSV1-UL19-1307 | + | GAGCUGAUGAUGCAAGGCCA | 20 | 1693 |
| HSV1-UL19-1006 | + | CGUCAGACGGGGCACGGCCA | 20 | 1392 |
| HSV1-UL19-545 | − | GUCCCGGGCCUUCGCGGCCA | 20 | 931 |
| HSV1-UL19-1157 | + | CUGGACCACGAGCCGGGCCA | 20 | 1543 |
| HSV1-UL19-1142 | + | GGCGAUGGUGGCGGGGGCCA | 20 | 1528 |
| HSV1-UL19-1171 | + | GUCAUCAACCAGCUGGGCCA | 20 | 1557 |
| HSV1-UL19-496 | − | CUACACCGCCGUCGUGGCCA | 20 | 882 |
| HSV1-UL19-1040 | + | GGCGUAGAUGCGCUUCUCCA | 20 | 1426 |
| HSV1-UL19-1051 | + | CAGGACGAACGUCAGGUCCA | 20 | 1437 |
| HSV1-UL19-1190 | + | GCAGUCGCGAUGGCGGUCCA | 20 | 1576 |
| HSV1-UL19-565 | − | GACGGGUUCGGCGGUGUCCA | 20 | 951 |
| HSV1-UL19-1400 | + | GACGUAAAGAACUUAAAGCA | 20 | 1786 |
| HSV1-UL19-1248 | + | CAGCACCUGCAGCGUGAGCA | 20 | 1634 |
| HSV1-UL19-520 | − | CCCAGGUGCCGCGGCGCGCA | 20 | 906 |
| HSV1-UL19-1287 | + | CUGCCGCGACAGGUCGCGCA | 20 | 1673 |
| HSV1-UL19-1028 | + | CACGUCGUCCAGACUUCGCA | 20 | 1414 |
| HSV1-UL19-193 | − | CGGGGCGUUUGAGCGCGGCA | 20 | 579 |
| HSV1-UL19-1007 | + | GGCAUGCGUCAGACGGGGCA | 20 | 1393 |
| HSV1-UL19-238 | − | CACGGCGCUCGUGAUGGGCA | 20 | 624 |
| HSV1-UL19-208 | − | GACGAGCUUUUUCCUGGGCA | 20 | 594 |
| HSV1-UL19-1308 | + | CGUCUUGAGCUGAUGAUGCA | 20 | 1694 |
| HSV1-UL19-1220 | + | CGGGACCACCAUGUUCUGCA | 20 | 1606 |
| HSV1-UL19-363 | − | AGACCUUCCCGAGGAGUGCA | 20 | 749 |
| HSV1-UL19-1239 | + | CACCAGAUUGGCCGGGUGCA | 20 | 1625 |
| HSV1-UL19-1211 | + | GUAGUAAAUCUUGUGGUGCA | 20 | 1597 |
| HSV1-UL19-1371 | + | GCGCGUCCCCGGGGGUUGCA | 20 | 1757 |
| HSV1-UL19-1188 | + | CAGGGCCGCGCGCCGCAUCA | 20 | 1574 |
| HSV1-UL19-579 | − | CUAUGACGCAUCCCCGCUCA | 20 | 965 |
| HSV1-UL19-463 | − | CGCGCUGACCUACGCGCUCA | 20 | 849 |
| HSV1-UL19-1233 | + | GGGGUGCGCGGGGUCCGUCA | 20 | 1619 |
| HSV1-UL19-1175 | + | GCCCAGCUCCGGGCCGGUCA | 20 | 1561 |
| HSV1-UL19-261 | − | GGGCCUGUUCAAUCCGGUCA | 20 | 647 |
| HSV1-UL19-235 | − | GAACGGGGCCAACCUGGUCA | 20 | 621 |
| HSV1-UL19-468 | − | CUUGCAUCAUCAGCUCAAGA | 20 | 854 |
| HSV1-UL19-1010 | + | GCGUGUCGGCAUGCGUCAGA | 20 | 1396 |
| HSV1-UL19-1215 | + | GCAGCAGCGGCCCCGCGAGA | 20 | 1601 |
| HSV1-UL19-230 | − | GCCGGUGACGUACGGCGAGA | 20 | 616 |
| HSV1-UL19-1342 | + | CUGCCGCGUUGUCCAGGAGA | 20 | 1728 |
| HSV1-UL19-561 | − | GGAGCGCCUGAUCGUGGAGA | 20 | 947 |
| HSV1-UL19-395 | − | GCGGACUUCAACCGCAACGA | 20 | 781 |
| HSV1-UL19-1186 | + | GGCGUCACAGUCCCACACGA | 20 | 1572 |
| HSV1-UL19-1326 | + | GACGGUUGCCGUGGCCACGA | 20 | 1712 |
| HSV1-UL19-1302 | + | CUCGCGGACGUGCUGCACGA | 20 | 1688 |
| HSV1-UL19-1313 | + | AAAGCGGUCCUGUCGGACGA | 20 | 1699 |
| HSV1-UL19-1039 | + | AAAGACCAGCUUCUCGCCGA | 20 | 1425 |
| HSV1-UL19-299 | − | UGGCCCACGGGCGGGUCCGA | 20 | 685 |
| HSV1-UL19-1088 | + | GCGUCAACGUUCAUCAGCGA | 20 | 1474 |
| HSV1-UL19-1388 | + | CGGGUUGGCCGUGGCCGCGA | 20 | 1774 |
| HSV1-UL19-1193 | + | CGCUAACCCGGCAGUCGCGA | 20 | 1579 |
| HSV1-UL19-1148 | + | CGCCCCGCGAACGGCGGCGA | 20 | 1534 |
| HSV1-UL19-999 | + | GUCCACGAGCCAGGCCUCGA | 20 | 1385 |
| HSV1-UL19-223 | − | ACGCGCGGGCGGCCGGUCGA | 20 | 609 |
| HSV1-UL19-439 | − | ACCAACAUGCGCAUAUUCGA | 20 | 825 |
| HSV1-UL19-1093 | + | GUACGGAUUGGCGGCUUCGA | 20 | 1479 |
| HSV1-UL19-538 | − | GUUUACGCGGGGGACAAGGA | 20 | 924 |
| HSV1-UL19-1020 | + | GGUGUCCUCCACUUUCAGGA | 20 | 1406 |
| HSV1-UL19-976 | + | GCGCUCAAACGCCCCGAGGA | 20 | 1362 |
| HSV1-UL19-1429 | + | GUUGCGGGCACUGCGGAGGA | 20 | 1815 |
| HSV1-UL19-1004 | + | AGACGGGGCACGGCCACGGA | 20 | 1390 |
| HSV1-UL19-302 | − | CGGGUCCGAUGGGUCGCGGA | 20 | 688 |
| HSV1-UL19-1328 | + | GUCGGUGACGGGGUUGCGGA | 20 | 1714 |
| HSV1-UL19-521 | − | GGUGCCGCGGCGCGCAGGGA | 20 | 907 |
| HSV1-UL19-1310 | + | CGGUGAACCCAAACCCGGGA | 20 | 1696 |
| HSV1-UL19-1385 | + | GUGGCCGCGAAGGCCCGGGA | 20 | 1771 |
| HSV1-UL19-1075 | + | AAACAGCUGGCGGGGCGGGA | 20 | 1461 |
| HSV1-UL19-1165 | + | CGGCCAUGCACUCCUCGGGA | 20 | 1551 |
| HSV1-UL19-1353 | + | GGCGCACCCGAACACGGGGA | 20 | 1739 |
| HSV1-UL19-1271 | + | GAACAGCGCGUGGACGGGGA | 20 | 1657 |
| HSV1-UL19-272 | − | CCAGCUGUUUUUUUGGGGGA | 20 | 658 |
| HSV1-UL19-1334 | + | CCAGGUAAAAGUUUUGGGGA | 20 | 1720 |
| HSV1-UL19-429 | − | AACGGGCGCGUGGUAGUGGA | 20 | 815 |
| HSV1-UL19-1275 | + | CCCGGCGAACAGCGCGUGGA | 20 | 1661 |
| HSV1-UL19-3212 | + | GUUGCGAGCGAGCUGUUGGA | 20 | 7811 |
| HSV1-UL19-1267 | + | AAAAUAGUCGCCAUUUUGGA | 20 | 1653 |
| HSV1-UL19-1380 | + | CUGGCCGUGGUCGUACAUGA | 20 | 1766 |
| HSV1-UL19-443 | − | GCACGCCGGAAUCCUGCUGA | 20 | 829 |
| HSV1-UL19-407 | − | CAAGAUUUACUACUACGUGA | 20 | 793 |
| HSV1-UL19-420 | − | GUGCCCCAGCGACCCCGUGA | 20 | 806 |
| HSV1-UL19-236 | − | CCUGGUCACGGCGCUCGUGA | 20 | 622 |
| HSV1-UL19-1332 | + | AAGGUUGCCCAUGUCGGUGA | 20 | 1718 |
| HSV1-UL19-229 | − | GCCGACGUGCCGGUGACGUA | 20 | 615 |
| HSV1-UL19-1096 | + | GCCGCCACGUACGCCCCGUA | 20 | 1482 |
| HSV1-UL19-283 | − | GUCGAAGCCGCCAAUCCGUA | 20 | 669 |
| HSV1-UL19-548 | − | GCGUCGCAGCGAUUUUCGUA | 20 | 934 |
| HSV1-UL19-1045 | + | AUGGCGCCCACCAGGGGGUA | 20 | 1431 |
| HSV1-UL19-1266 | + | AGUCGCCAUUUUGGAUGGUA | 20 | 1652 |
| HSV1-UL19-1156 | + | GACGUGCUCGCUGCCGUGUA | 20 | 1542 |
| HSV1-UL19-1089 | + | CUUCGACGGGGUCGCGGUUA | 20 | 1475 |
| HSV1-UL19-483 | − | CAGGUGGCCCGGCACGAAAC | 20 | 869 |
| HSV1-UL19-1420 | + | GCGGGUAGGCCUCCUGAAAC | 20 | 1806 |
| HSV1-UL19-426 | − | UCAACGCCAUGUUUCACAAC | 20 | 812 |
| HSV1-UL19-575 | − | UCCUCCGCAGUGCCCGCAAC | 20 | 961 |
| HSV1-UL19-40 | + | GCUGUUGGAUGGCGCGCAAC | 20 | 421 |
| HSV1-UL19-555 | − | ACGGGGCCUACCACCUCAAC | 20 | 941 |
| HSV1-UL19-245 | − | AGGAGCAGCUCGACCUGAAC | 20 | 631 |
| HSV1-UL19-232 | − | ACGGCGAGAUGGUCCUGAAC | 20 | 618 |
| HSV1-UL19-1057 | + | GUUCCAUGACCGGAUUGAAC | 20 | 1443 |
| HSV1-UL19-552 | − | ACGGGGACCUGCUCUAUAAC | 20 | 938 |
| HSV1-UL19-1355 | + | ACCUGGGCGCACCCGAACAC | 20 | 1741 |
| HSV1-UL19-1418 | + | CCUCCUGAAACAGGCCACAC | 20 | 1804 |
| HSV1-UL19-1366 | + | UAGUUGACGUCGGUCGACAC | 20 | 1752 |
| HSV1-UL19-1197 | + | UUACAUGCCGCCGCGUACAC | 20 | 1583 |
| HSV1-UL19-1391 | + | AAAAUCGCUGCGACGCCCAC | 20 | 1777 |
| HSV1-UL19-296 | − | GGGGGCAGCGCCUGGCCCAC | 20 | 682 |
| HSV1-UL19-1306 | + | AGCUGAUGAUGCAAGGCCAC | 20 | 1692 |
| HSV1-UL19-1398 | + | UUAAAGCAGGGGCUGAGCAC | 20 | 1784 |
| HSV1-UL19-400 | − | CCGCGGAUGACCGGCCGCAC | 20 | 786 |
| HSV1-UL19-1161 | + | UCCAGUAGCUGGUGAUGCAC | 20 | 1547 |
| HSV1-UL19-1024 | + | ACCAUCUCGCCGUACGUCAC | 20 | 1410 |
| HSV1-UL19-1232 | + | GGGUGCGCGGGGUCCGUCAC | 20 | 1618 |
| HSV1-UL19-469 | − | UUGCAUCAUCAGCUCAAGAC | 20 | 855 |
| HSV1-UL19-1009 | + | CGUGUCGGCAUGCGUCAGAC | 20 | 1395 |
| HSV1-UL19-1084 | + | CGAUCGCGUGUUCCAGAGAC | 20 | 1470 |
| HSV1-UL19-562 | − | GAGCGCCUGAUCGUGGAGAC | 20 | 948 |
| HSV1-UL19-1301 | + | UCGCGGACGUGCUGCACGAC | 20 | 1687 |
| HSV1-UL19-474 | − | UGGGUUCACCGUCGUCCGAC | 20 | 860 |
| HSV1-UL19-1289 | + | CCAGGGGGACCUGCCGCGAC | 20 | 1675 |
| HSV1-UL19-1012 | + | GUGACGAGCACCCCGUCGAC | 20 | 1398 |
| HSV1-UL19-224 | − | CGCGCGGGCGGCCGGUCGAC | 20 | 610 |
| HSV1-UL19-440 | − | CCAACAUGCGCAUAUUCGAC | 20 | 826 |
| HSV1-UL19-1092 | + | UACGGAUUGGCGGCUUCGAC | 20 | 1478 |
| HSV1-UL19-406 | − | GGCCGCACCGGGGGGCGGAC | 20 | 792 |
| HSV1-UL19-581 | − | CGCAUCCCCGCUCAAGGGAC | 20 | 967 |
| HSV1-UL19-1384 | + | UGGCCGCGAAGGCCCGGGAC | 20 | 1770 |
| HSV1-UL19-1352 | + | GCGCACCCGAACACGGGGAC | 20 | 1738 |
| HSV1-UL19-430 | − | ACGGGCGCGUGGUAGUGGAC | 20 | 816 |
| HSV1-UL19-1274 | + | CCGGCGAACAGCGCGUGGAC | 20 | 1660 |
| HSV1-UL19-1058 | + | GCGGCAAACCGUUCCAUGAC | 20 | 1444 |
| HSV1-UL19-399 | − | CCGCGGACGCCGCGGAUGAC | 20 | 785 |
| HSV1-UL19-371 | − | GUUGAUGACUUUACCCUGAC | 20 | 757 |
| HSV1-UL19-1331 | + | AGGUUGCCCAUGUCGGUGAC | 20 | 1717 |
| HSV1-UL19-354 | − | UCCAGUGCAUCACCAGCUAC | 20 | 740 |
| HSV1-UL19-284 | − | UCGAAGCCGCCAAUCCGUAC | 20 | 670 |
| HSV1-UL19-549 | − | CGUCGCAGCGAUUUUCGUAC | 20 | 935 |
| HSV1-UL19-364 | − | AGGAGUGCAUGGCCGUGUAC | 20 | 750 |
| HSV1-UL19-1312 | + | GACGACGGUGAACCCAAACC | 20 | 1698 |
| HSV1-UL19-304 | − | GCAGCCCGACAACGCCAACC | 20 | 690 |
| HSV1-UL19-234 | − | GGUCCUGAACGGGGCCAACC | 20 | 620 |
| HSV1-UL19-1194 | + | CGUGGCCCCCCGCGCUAACC | 20 | 1580 |
| HSV1-UL19-1050 | + | UCAGGUCCAUGGCGCCCACC | 20 | 1436 |
| HSV1-UL19-1170 | + | GGGCCUCGACGUGCGCCACC | 20 | 1556 |
| HSV1-UL19-325 | − | CGGGGGAGAUCCAGGCCACC | 20 | 711 |
| HSV1-UL19-267 | − | AGUCCCCGCCCCCGGCCACC | 20 | 653 |
| HSV1-UL19-1318 | + | CAGUUUCGUGCCGGGCCACC | 20 | 1704 |
| HSV1-UL19-200 | − | ACAACGGCCGCCUGGCCACC | 20 | 586 |
| HSV1-UL19-1083 | + | CCAGAGACAGGCGCAGCACC | 20 | 1469 |
| HSV1-UL19-401 | − | CGCGGAUGACCGGCCGCACC | 20 | 787 |
| HSV1-UL19-1358 | + | UCCCUGCGCGCCGCGGCACC | 20 | 1744 |
| HSV1-UL19-421 | − | CCCCGCGCACCCCCUGCACC | 20 | 807 |
| HSV1-UL19-378 | − | GAAUCACCUAAUGCGAGACC | 20 | 764 |
| HSV1-UL19-993 | + | UUCGCUUUAGCUCGGCGACC | 20 | 1379 |
| HSV1-UL19-366 | − | CAUGGCCGUGUACCGGGACC | 20 | 752 |
| HSV1-UL19-1293 | + | CCCCCAGAGCCGGGGGGACC | 20 | 1679 |
| HSV1-UL19-491 | − | GCCGCGCGCGAACGUGGACC | 20 | 877 |
| HSV1-UL19-1027 | + | CCAUCACGAGCGCCGUGACC | 20 | 1413 |
| HSV1-UL19-197 | − | GUUGCCGAUGCAACGAUACC | 20 | 583 |
| HSV1-UL19-347 | − | GUUCGACGACCGCAACUACC | 20 | 733 |
| HSV1-UL19-365 | − | GGAGUGCAUGGCCGUGUACC | 20 | 751 |
| HSV1-UL19-499 | − | CCUUCCCCAAAACUUUUACC | 20 | 885 |
| HSV1-UL19-1311 | + | ACGACGGUGAACCCAAACCC | 20 | 1697 |
| HSV1-UL19-396 | − | CCAGCUGCUGCACAACACCC | 20 | 782 |
| HSV1-UL19-989 | + | CAGGGUCGCCCGGGCCACCC | 20 | 1375 |
| HSV1-UL19-1416 | + | UCCACGAGUUCGCGGCACCC | 20 | 1802 |
| HSV1-UL19-205 | − | CAGGGUGGCCCGGGCGACCC | 20 | 591 |
| HSV1-UL19-303 | − | CGCGGAAGGCCAGAUGACCC | 20 | 689 |
| HSV1-UL19-525 | − | UCCGCCGGCCCUGCAACCCC | 20 | 911 |
| HSV1-UL19-1415 | + | CCACGAGUUCGCGGCACCCC | 20 | 1801 |
| HSV1-UL19-526 | − | CCGCCGGCCCUGCAACCCCC | 20 | 912 |
| HSV1-UL19-254 | − | CACCAACGUUCCCUACCCCC | 20 | 640 |
| HSV1-UL19-1122 | + | GCCGGGGGAACGUCCCCCCC | 20 | 1508 |
| HSV1-UL19-567 | − | GUACAGUUUAAGCGCCCCCC | 20 | 953 |
| HSV1-UL19-1210 | + | GCACGGUCCAGUCCGCCCCC | 20 | 1596 |
| HSV1-UL19-566 | − | CGUACAGUUUAAGCGCCCCC | 20 | 952 |
| HSV1-UL19-1105 | + | GUGGGCCAGGCGCUGCCCCC | 20 | 1491 |
| HSV1-UL19-1128 | + | AGGUGGCCUGGAUCUCCCCC | 20 | 1514 |
| HSV1-UL19-453 | − | CCUGUCGCGGCAGGUCCCCC | 20 | 839 |
| HSV1-UL19-454 | − | GCAGGUCCCCCUGGUCCCCC | 20 | 840 |
| HSV1-UL19-450 | − | GAACGCGCCCAAUUUCCCCC | 20 | 836 |
| HSV1-UL19-1378 | + | GUCCCCCGCGUAAACGCCCC | 20 | 1764 |
| HSV1-UL19-266 | − | GGGGACCUAGUCCCCGCCCC | 20 | 652 |
| HSV1-UL19-196 | − | GCUGGAAAAGGCGCCGCCCC | 20 | 582 |
| HSV1-UL19-418 | − | GUGGUCCCGGAGAUCGCCCC | 20 | 804 |
| HSV1-UL19-1138 | + | CACCCCCAGCUCCAGGCCCC | 20 | 1524 |
| HSV1-UL19-1348 | + | GGACGGGCUGGGCCGGCCCC | 20 | 1734 |
| HSV1-UL19-258 | − | CCUGACGUUCGUCCUGCCCC | 20 | 644 |
| HSV1-UL19-328 | − | GGUGAACGGCAACUUGCCCC | 20 | 714 |
| HSV1-UL19-1129 | + | CAGGUGGCCUGGAUCUCCCC | 20 | 1515 |
| HSV1-UL19-1375 | + | GCCCCCGGCGGCGCGUCCCC | 20 | 1761 |
| HSV1-UL19-318 | − | GCCGGGGGGGGACGUUCCCC | 20 | 704 |
| HSV1-UL19-1324 | + | CGGCGGUGUAGCCCACGCCC | 20 | 1710 |
| HSV1-UL19-334 | − | GGCGGCGUUCCGGGACGCCC | 20 | 720 |
| HSV1-UL19-351 | − | CGAGCACGUCUUCUGCGCCC | 20 | 737 |
| HSV1-UL19-517 | − | CCCCGUGUUCGGGUGCGCCC | 20 | 903 |
| HSV1-UL19-990 | + | CUCGGCGACCAGGGUCGCCC | 20 | 1376 |
| HSV1-UL19-1386 | + | GGCCGUGGCCGCGAAGGCCC | 20 | 1772 |
| HSV1-UL19-1056 | + | UGACCGGAUUGAACAGGCCC | 20 | 1442 |
| HSV1-UL19-1139 | + | CCACCCCCAGCUCCAGGCCC | 20 | 1525 |
| HSV1-UL19-369 | − | GGUGGCGCACGUCGAGGCCC | 20 | 755 |
| HSV1-UL19-253 | − | GCUGGUCUUUCUGGAGGCCC | 20 | 639 |
| HSV1-UL19-321 | − | GACGUUCCCCCGGCCGGCCC | 20 | 707 |
| HSV1-UL19-1338 | + | GAGGGGGAGCCCCGCGGCCC | 20 | 1724 |
| HSV1-UL19-383 | − | CCUGAUGCGGCGCGCGGCCC | 20 | 769 |
| HSV1-UL19-288 | − | CGGGGCGUACGUGGCGGCCC | 20 | 674 |
| HSV1-UL19-1277 | + | GUUCGCCACGUGGUCGGCCC | 20 | 1663 |
| HSV1-UL19-1316 | + | GGGCCACCUGGAGCUGGCCC | 20 | 1702 |
| HSV1-UL19-204 | − | CCUGGCCACCAGGGUGGCCC | 20 | 590 |
| HSV1-UL19-995 | + | GGCGGUGGCCCGCCUUGCCC | 20 | 1381 |
| HSV1-UL19-471 | − | UCAAGACGGGCCUCCAUCCC | 20 | 857 |
| HSV1-UL19-1304 | + | GUAGGUCAGCGCGUUCUCCC | 20 | 1690 |
| HSV1-UL19-543 | − | CGGCCAGAGCGACCCGUCCC | 20 | 929 |
| HSV1-UL19-1376 | + | CGCCCCCGGCGGCGCGUCCC | 20 | 1762 |
| HSV1-UL19-1060 | + | GGGGGCGGGGACUAGGUCCC | 20 | 1446 |
| HSV1-UL19-983 | + | GCAUCGGCAACAACAAAGCC | 20 | 1369 |
| HSV1-UL19-1117 | + | CGGGGUGCAGCUCAAGAGCC | 20 | 1503 |
| HSV1-UL19-1297 | + | AGUAGUUGGCCCCCAGAGCC | 20 | 1683 |
| HSV1-UL19-1159 | + | GAUGCACUGGACCACGAGCC | 20 | 1545 |
| HSV1-UL19-1000 | + | CGUGGUGAGGUCCACGAGCC | 20 | 1386 |
| HSV1-UL19-290 | − | AGCAGCUGUUUUUGAACGCC | 20 | 676 |
| HSV1-UL19-264 | − | AACGGUUUGCCGCGCACGCC | 20 | 650 |
| HSV1-UL19-333 | − | CGGCGGCGUUCCGGGACGCC | 20 | 719 |
| HSV1-UL19-511 | − | GGUCGUGGCGGGAAACCGCC | 20 | 897 |
| HSV1-UL19-1152 | + | GCAGCAGGUAAAACACCGCC | 20 | 1538 |
| HSV1-UL19-212 | − | GGGCAAGGCGGGCCACCGCC | 20 | 598 |
| HSV1-UL19-273 | − | UUUUUGGGGGAAGGACCGCC | 20 | 659 |
| HSV1-UL19-529 | − | ACCCCCGGGGACGCGCCGCC | 20 | 915 |
| HSV1-UL19-199 | − | AUACCUGGACAACGGCCGCC | 20 | 585 |
| HSV1-UL19-1182 | + | CGAGGGGUGGCAGCAGCGCC | 20 | 1568 |
| HSV1-UL19-294 | − | GAACGCCUGGGGGCAGCGCC | 20 | 680 |
| HSV1-UL19-1135 | + | ACGCCGCCGGACAUAGCGCC | 20 | 1521 |
| HSV1-UL19-1132 | + | GUUGCCGUUCACCACGCGCC | 20 | 1518 |
| HSV1-UL19-1226 | + | UCGCUGGGGCACUCCUCGCC | 20 | 1612 |
| HSV1-UL19-991 | + | GCUCGGCGACCAGGGUCGCC | 20 | 1377 |
| HSV1-UL19-447 | − | CCGUCCACGCGCUGUUCGCC | 20 | 833 |
| HSV1-UL19-1387 | + | UGGCCGUGGCCGCGAAGGCC | 20 | 1773 |
| HSV1-UL19-215 | − | GCCGGGAGGCCGUCGAGGCC | 20 | 601 |
| HSV1-UL19-523 | − | CGCAGGGAUGGACCACGGCC | 20 | 909 |
| HSV1-UL19-975 | + | CAAACGCCCCGAGGACGGCC | 20 | 1361 |
| HSV1-UL19-372 | − | UGACUUUACCCUGACCGGCC | 20 | 758 |
| HSV1-UL19-320 | − | GGACGUUCCCCCGGCCGGCC | 20 | 706 |
| HSV1-UL19-1125 | + | GGAUCUCCCCCGGGCCGGCC | 20 | 1511 |
| HSV1-UL19-1098 | + | UGUCUGCGGCGGGGCCGGCC | 20 | 1484 |
| HSV1-UL19-461 | − | ACGUCCGCGAGAGCGCGGCC | 20 | 847 |
| HSV1-UL19-1285 | + | GCGACAGGUCGCGCAGGGCC | 20 | 1671 |
| HSV1-UL19-1158 | + | ACUGGACCACGAGCCGGGCC | 20 | 1544 |
| HSV1-UL19-1203 | + | CGGCGUCCGCGGCUCGGGCC | 20 | 1589 |
| HSV1-UL19-336 | − | GUUCCGGGACGCCCGGGGCC | 20 | 722 |
| HSV1-UL19-1172 | + | AGUCAUCAACCAGCUGGGCC | 20 | 1558 |
| HSV1-UL19-1106 | + | AUCGGACCCGCCCGUGGGCC | 20 | 1492 |
| HSV1-UL19-352 | − | ACGUCUUCUGCGCCCUGGCC | 20 | 738 |
| HSV1-UL19-1130 | + | UCACCACGCGCCAGGUGGCC | 20 | 1516 |
| HSV1-UL19-482 | − | UGGGCCAGCUCCAGGUGGCC | 20 | 868 |
| HSV1-UL19-203 | − | GCCUGGCCACCAGGGUGGCC | 20 | 589 |
| HSV1-UL19-1067 | + | GCGCGGGGAUCCGGGUGGCC | 20 | 1453 |
| HSV1-UL19-987 | + | CCCGGGCCACCCUGGUGGCC | 20 | 1373 |
| HSV1-UL19-1241 | + | UGUUGGCCACCAGAUUGGCC | 20 | 1627 |
| HSV1-UL19-385 | − | GGACCGCCAUCGCGACUGCC | 20 | 771 |
| HSV1-UL19-312 | − | GUGGCCGACGUGGAGCUGCC | 20 | 698 |
| HSV1-UL19-559 | − | CGCCGCCAAACAUCGCUGCC | 20 | 945 |
| HSV1-UL19-1319 | + | CCCCCCGCCAGUUUCGUGCC | 20 | 1705 |
| HSV1-UL19-470 | − | CUCAAGACGGGCCUCCAUCC | 20 | 856 |
| HSV1-UL19-324 | − | GGCCGGCCCGGGGGAGAUCC | 20 | 710 |
| HSV1-UL19-1070 | + | GCGGGAAGGCGCGGGGAUCC | 20 | 1456 |
| HSV1-UL19-998 | + | GCCAGGCCUCGACGGCCUCC | 20 | 1384 |
| HSV1-UL19-1140 | + | GGCGUCCCACCCCCAGCUCC | 20 | 1526 |
| HSV1-UL19-1178 | + | GCGCCUGCCCGCCCAGCUCC | 20 | 1564 |
| HSV1-UL19-480 | − | GUACUUCCUGGGCCAGCUCC | 20 | 866 |
| HSV1-UL19-1405 | + | UCUCCACGAUCAGGCGCUCC | 20 | 1791 |
| HSV1-UL19-1112 | + | CGGGCUGCAUAAACUGCUCC | 20 | 1498 |
| HSV1-UL19-1222 | + | CCUCGCCGGGGGCGAUCUCC | 20 | 1608 |
| HSV1-UL19-504 | − | CCGCGGGGCUCCCCCUCUCC | 20 | 890 |
| HSV1-UL19-1041 | + | CGGCGUAGAUGCGCUUCUCC | 20 | 1427 |
| HSV1-UL19-3217 | − | ACAGCUCGCUCGCAACGUCC | 20 | 7816 |
| HSV1-UL19-542 | − | ACGGCCAGAGCGACCCGUCC | 20 | 928 |
| HSV1-UL19-1361 | + | UAAACUCACACACGGCGUCC | 20 | 1747 |
| HSV1-UL19-1137 | + | GCUCCAGGCCCCGGGCGUCC | 20 | 1523 |
| HSV1-UL19-1169 | + | CGACGUGCGCCACCAGGUCC | 20 | 1555 |
| HSV1-UL19-1191 | + | GGCAGUCGCGAUGGCGGUCC | 20 | 1577 |
| HSV1-UL19-417 | − | CCUGCAGAACAUGGUGGUCC | 20 | 803 |
| HSV1-UL19-985 | + | UGGCCAGGCGGCCGUUGUCC | 20 | 1371 |
| HSV1-UL19-1344 | + | ACACGGCUGCCGCGUUGUCC | 20 | 1730 |
| HSV1-UL19-478 | − | CGCGUCGGAGGCGUACUUCC | 20 | 864 |
| HSV1-UL19-332 | − | GCUAUGUCCGGCGGCGUUCC | 20 | 718 |
| HSV1-UL19-206 | − | CUGCGAGACGAGCUUUUUCC | 20 | 592 |
| HSV1-UL19-1401 | + | CGACGUAAAGAACUUAAAGC | 20 | 1787 |
| HSV1-UL19-250 | − | GGUGUCCAUCGGCGAGAAGC | 20 | 636 |
| HSV1-UL19-1081 | + | CCUUCCCCCAAAAAAACAGC | 20 | 1467 |
| HSV1-UL19-1174 | + | GGGUAAAGUCAUCAACCAGC | 20 | 1560 |
| HSV1-UL19-370 | − | CGUCGAGGCCCUGGCCCAGC | 20 | 756 |
| HSV1-UL19-1031 | + | GCUCCUCCUGCAUCUCCAGC | 20 | 1417 |
| HSV1-UL19-1201 | + | CCUGGGUGUUGUGCAGCAGC | 20 | 1587 |
| HSV1-UL19-1154 | + | CGUGUAUGGCGGCCUGCAGC | 20 | 1540 |
| HSV1-UL19-1260 | + | GAUGCUGGGGGGCCAUCAGC | 20 | 1646 |
| HSV1-UL19-1298 | + | AAGUAGUUGGCCCCCAGAGC | 20 | 1684 |
| HSV1-UL19-1393 | + | GGUAGGCCCCGUUAUAGAGC | 20 | 1779 |
| HSV1-UL19-1160 | + | UGAUGCACUGGACCACGAGC | 20 | 1546 |
| HSV1-UL19-373 | − | UACCCUGACCGGCCCGGAGC | 20 | 759 |
| HSV1-UL19-1317 | + | CGUGCCGGGCCACCUGGAGC | 20 | 1703 |
| HSV1-UL19-337 | − | GGACGCCCGGGGCCUGGAGC | 20 | 723 |
| HSV1-UL19-1422 | + | GGUCGCUGGCGCAGGUGAGC | 20 | 1808 |
| HSV1-UL19-1440 | + | ACAGAGCCAGUCCCUUGAGC | 20 | 1826 |
| HSV1-UL19-1162 | + | CCGCGUGUUGUUCCAGUAGC | 20 | 1548 |
| HSV1-UL19-1119 | + | CCCACAAAGAAGUCGAACGC | 20 | 1505 |
| HSV1-UL19-263 | − | GAACGGUUUGCCGCGCACGC | 20 | 649 |
| HSV1-UL19-442 | − | UUCGACGGGGCGUUGCACGC | 20 | 828 |
| HSV1-UL19-246 | − | CGACCUGAACCGGCAGACGC | 20 | 632 |
| HSV1-UL19-438 | − | UGCUCGGCGGCGCCCGACGC | 20 | 824 |
| HSV1-UL19-533 | − | GCCGCCGGGGGCGUUUACGC | 20 | 919 |
| HSV1-UL19-1153 | + | UGCAGCAGGUAAAACACCGC | 20 | 1539 |
| HSV1-UL19-211 | − | UGGGCAAGGCGGGCCACCGC | 20 | 597 |
| HSV1-UL19-412 | − | CGGGGCCGCUGCUGCACCGC | 20 | 798 |
| HSV1-UL19-1136 | + | CGGGCGUCCCGGAACGCCGC | 20 | 1522 |
| HSV1-UL19-528 | − | AACCCCCGGGGACGCGCCGC | 20 | 914 |
| HSV1-UL19-502 | − | AAAACUUUUACCUGGGCCGC | 20 | 888 |
| HSV1-UL19-524 | − | CCGACGUCAACUACUUCCGC | 20 | 910 |
| HSV1-UL19-1346 | + | CCGCCACGACCGCGUUCCGC | 20 | 1732 |
| HSV1-UL19-1183 | + | ACGAGGGGUGGCAGCAGCGC | 20 | 1569 |
| HSV1-UL19-387 | − | CGCGACUGCCGGGUUAGCGC | 20 | 773 |
| HSV1-UL19-220 | − | UGACGCAUGCCGACACGCGC | 20 | 606 |
| HSV1-UL19-578 | − | CAACGGGGAAGCCCACGCGC | 20 | 964 |
| HSV1-UL19-519 | − | GCCCAGGUGCCGCGGCGCGC | 20 | 905 |
| HSV1-UL19-1288 | + | CCUGCCGCGACAGGUCGCGC | 20 | 1674 |
| HSV1-UL19-1073 | + | CUGGCGGGGCGGGAAGGCGC | 20 | 1459 |
| HSV1-UL19-1424 | + | GAGGGCGGGGUCGCUGGCGC | 20 | 1810 |
| HSV1-UL19-1235 | + | GCCGGGUGCAGGGGGUGCGC | 20 | 1621 |
| HSV1-UL19-357 | − | GUUCGUGAACGACUACUCGC | 20 | 743 |
| HSV1-UL19-557 | − | CCACCUCAACGGGGCCUCGC | 20 | 943 |
| HSV1-UL19-1227 | + | GUCGCUGGGGCACUCCUCGC | 20 | 1613 |
| HSV1-UL19-409 | − | GAUGGUGCCCGCCUUCUCGC | 20 | 795 |
| HSV1-UL19-1411 | + | GCGCUUAAACUGUACGUCGC | 20 | 1797 |
| HSV1-UL19-1230 | + | GGGGUCCGUCACGGGGUCGC | 20 | 1616 |
| HSV1-UL19-1425 | + | GCGGAGGAGGGCGGGGUCGC | 20 | 1811 |
| HSV1-UL19-345 | − | CCACCAUCGCCGCCGUUCGC | 20 | 731 |
| HSV1-UL19-446 | − | CCCGUCCACGCGCUGUUCGC | 20 | 832 |
| HSV1-UL19-210 | − | AGCUUUUUCCUGGGCAAGGC | 20 | 596 |
| HSV1-UL19-1213 | + | CAGCGGCCCCGCGAGAAGGC | 20 | 1599 |
| HSV1-UL19-319 | − | GGGGGGGACGUUCCCCCGGC | 20 | 705 |
| HSV1-UL19-289 | − | GCGUACGUGGCGGCCCCGGC | 20 | 675 |
| HSV1-UL19-1126 | + | UGGAUCUCCCCCGGGCCGGC | 20 | 1512 |
| HSV1-UL19-1099 | + | AUGUCUGCGGCGGGGCCGGC | 20 | 1485 |
| HSV1-UL19-460 | − | CACGUCCGCGAGAGCGCGGC | 20 | 846 |
| HSV1-UL19-1322 | + | GGUCCACGUUCGCGCGCGGC | 20 | 1708 |
| HSV1-UL19-452 | − | CCUGCGCGACCUGUCGCGGC | 20 | 838 |
| HSV1-UL19-222 | − | UGCCGACACGCGCGGGCGGC | 20 | 608 |
| HSV1-UL19-1102 | + | AGCUGCUGCAUGUCUGCGGC | 20 | 1488 |
| HSV1-UL19-1208 | + | AGUCCGCCCCCCGGUGCGGC | 20 | 1594 |
| HSV1-UL19-1286 | + | CGCGACAGGUCGCGCAGGGC | 20 | 1672 |
| HSV1-UL19-1370 | + | GUCCCCGGGGGUUGCAGGGC | 20 | 1756 |
| HSV1-UL19-1427 | + | CGGGCACUGCGGAGGAGGGC | 20 | 1813 |
| HSV1-UL19-298 | − | GCAGCGCCUGGCCCACGGGC | 20 | 684 |
| HSV1-UL19-1351 | + | ACCCGAACACGGGGACGGGC | 20 | 1737 |
| HSV1-UL19-1127 | + | GGCCUGGAUCUCCCCCGGGC | 20 | 1513 |
| HSV1-UL19-1177 | + | CUGCCCGCCCAGCUCCGGGC | 20 | 1563 |
| HSV1-UL19-377 | − | CGGCCCGGAGCUGGGCGGGC | 20 | 763 |
| HSV1-UL19-1053 | + | GAUUGAACAGGCCCAGGGGC | 20 | 1439 |
| HSV1-UL19-1100 | + | CUGCAUGUCUGCGGCGGGGC | 20 | 1486 |
| HSV1-UL19-1076 | + | AAAAAAACAGCUGGCGGGGC | 20 | 1462 |
| HSV1-UL19-1063 | + | GGAUCCGGGUGGCCGGGGGC | 20 | 1449 |
| HSV1-UL19-514 | − | GGCGGGAAACCGCCUGGGGC | 20 | 900 |
| HSV1-UL19-376 | − | UGACCGGCCCGGAGCUGGGC | 20 | 762 |
| HSV1-UL19-1349 | + | AACACGGGGACGGGCUGGGC | 20 | 1735 |
| HSV1-UL19-1246 | + | ACCUGCAGCGUGAGCAUGGC | 20 | 1632 |
| HSV1-UL19-465 | − | CUGACCUACGCGCUCAUGGC | 20 | 851 |
| HSV1-UL19-485 | − | UGGCCCGGCACGAAACUGGC | 20 | 871 |
| HSV1-UL19-1079 | + | CCCCCAAAAAAACAGCUGGC | 20 | 1465 |
| HSV1-UL19-510 | − | CUGCGGAACGCGGUCGUGGC | 20 | 896 |
| HSV1-UL19-1068 | + | GGCGCGGGGAUCCGGGUGGC | 20 | 1454 |
| HSV1-UL19-1145 | + | CGAACGGCGGCGAUGGUGGC | 20 | 1531 |
| HSV1-UL19-1242 | + | GUGUUGGCCACCAGAUUGGC | 20 | 1628 |
| HSV1-UL19-1265 | + | GGAUGGUAUGGUCCAGAUGC | 20 | 1651 |
| HSV1-UL19-243 | − | CCGCCACCUGCUGGAGAUGC | 20 | 629 |
| HSV1-UL19-384 | − | UGGACCGCCAUCGCGACUGC | 20 | 770 |
| HSV1-UL19-1019 | + | CCACUUUCAGGAAGGACUGC | 20 | 1405 |
| HSV1-UL19-242 | − | CGACGUGGGCCGCCACCUGC | 20 | 628 |
| HSV1-UL19-311 | − | GGUGGCCGACGUGGAGCUGC | 20 | 697 |
| HSV1-UL19-431 | − | GCCCGCCAUGCUCACGCUGC | 20 | 817 |
| HSV1-UL19-349 | − | GGCGGUGUUUUACCUGCUGC | 20 | 735 |
| HSV1-UL19-194 | − | CCAGAUGCUGCACGUGCUGC | 20 | 580 |
| HSV1-UL19-1033 | + | CGAGCUCGUCCAGCGUCUGC | 20 | 1419 |
| HSV1-UL19-1221 | + | CCGGGACCACCAUGUUCUGC | 20 | 1607 |
| HSV1-UL19-228 | − | GGACACCGAAGCCGACGUGC | 20 | 614 |
| HSV1-UL19-1320 | + | CCCCCCCGCCAGUUUCGUGC | 20 | 1706 |
| HSV1-UL19-432 | − | CAUGCUCACGCUGCAGGUGC | 20 | 818 |
| HSV1-UL19-1240 | + | CCACCAGAUUGGCCGGGUGC | 20 | 1626 |
| HSV1-UL19-1433 | + | CGCGUGGGCUUCCCCGUUGC | 20 | 1819 |
| HSV1-UL19-1372 | + | GGCGCGUCCCCGGGGGUUGC | 20 | 1758 |
| HSV1-UL19-422 | − | CCCCCUGCACCCGGCCAAUC | 20 | 808 |
| HSV1-UL19-260 | − | GCCCCUGGGCCUGUUCAAUC | 20 | 646 |
| HSV1-UL19-444 | − | GCUGAUGGCCCCCCAGCAUC | 20 | 830 |
| HSV1-UL19-979 | + | CCAGCAGCACGUGCAGCAUC | 20 | 1365 |
| HSV1-UL19-1189 | + | CCAGGGCCGCGCGCCGCAUC | 20 | 1575 |
| HSV1-UL19-1110 | + | UAAACUGCUCCGGGGUCAUC | 20 | 1496 |
| HSV1-UL19-1406 | + | CCGAACCCGUCUCCACGAUC | 20 | 1792 |
| HSV1-UL19-276 | − | UGUCUCUGGAACACGCGAUC | 20 | 662 |
| HSV1-UL19-248 | − | GACGCGCGUGCGCGCGGAUC | 20 | 634 |
| HSV1-UL19-1071 | + | GGCGGGAAGGCGCGGGGAUC | 20 | 1457 |
| HSV1-UL19-1016 | + | GCAGGAGGCGCUGUUUGAUC | 20 | 1402 |
| HSV1-UL19-359 | − | CGUACGUCGUGACCUACCUC | 20 | 745 |
| HSV1-UL19-1166 | + | UACACGGCCAUGCACUCCUC | 20 | 1552 |
| HSV1-UL19-190 | − | GCAACGUCCAGGCCGUCCUC | 20 | 576 |
| HSV1-UL19-1179 | + | UGCGCCUGCCCGCCCAGCUC | 20 | 1565 |
| HSV1-UL19-1383 | + | AGGCCCGGGACGGGUCGCUC | 20 | 1769 |
| HSV1-UL19-455 | − | CCCCCUGGUCCCCCCGGCUC | 20 | 841 |
| HSV1-UL19-1204 | + | AUCCGCGGCGUCCGCGGCUC | 20 | 1590 |
| HSV1-UL19-1113 | + | UCGGGCUGCAUAAACUGCUC | 20 | 1499 |
| HSV1-UL19-1223 | + | UCCUCGCCGGGGGCGAUCUC | 20 | 1609 |
| HSV1-UL19-274 | − | CCAGGUGCUGCGCCUGUCUC | 20 | 660 |
| HSV1-UL19-239 | − | GGGCAAGGCCGUGCGAAGUC | 20 | 625 |
| HSV1-UL19-1052 | + | CCAGGGGCAGGACGAACGUC | 20 | 1438 |
| HSV1-UL19-1252 | + | GCGGUGUUGGCGCCCGCGUC | 20 | 1638 |
| HSV1-UL19-1038 | + | CCGCGCGCACGCGCGUCGUC | 20 | 1424 |
| HSV1-UL19-1176 | + | CGCCCAGCUCCGGGCCGGUC | 20 | 1562 |
| HSV1-UL19-280 | − | UGAACGUUGACGCGGCGGUC | 20 | 666 |
| HSV1-UL19-329 | − | CUUGCCCCUGGCGCUAUGUC | 20 | 715 |
| HSV1-UL19-1114 | + | AGAGCCAGGUUGGCGUUGUC | 20 | 1500 |
| HSV1-UL19-1259 | + | GGGGGCCAUCAGCAGGAUUC | 20 | 1645 |
| HSV1-UL19-1025 | + | UGACCAGGUUGGCCCCGUUC | 20 | 1411 |
| HSV1-UL19-331 | − | CGCUAUGUCCGGCGGCGUUC | 20 | 717 |
| HSV1-UL19-1032 | + | CGUCCAGCGUCUGCCGGUUC | 20 | 1418 |
| HSV1-UL19-516 | − | CCCAGCCCGUCCCCGUGUUC | 20 | 902 |
| HSV1-UL19-1021 | + | CUUCGGUGUCCUCCACUUUC | 20 | 1407 |
| HSV1-UL19-251 | − | CGGCGAGAAGCUGGUCUUUC | 20 | 637 |
| HSV1-UL19-572 | − | GGACCCGUGUGGCCUGUUUC | 20 | 958 |
| HSV1-UL19-1315 | + | GCACGUUCUCAGUCACAAAG | 20 | 1701 |
| HSV1-UL19-226 | − | CCUGCAGUCCUUCCUGAAAG | 20 | 612 |
| HSV1-UL19-1438 | + | AGACGAGAUACUGCGCGAAG | 20 | 1824 |
| HSV1-UL19-1048 | + | AGGUCCAUGGCGCCCACCAG | 20 | 1434 |
| HSV1-UL19-1291 | + | CCCAGAGCCGGGGGGACCAG | 20 | 1677 |
| HSV1-UL19-1054 | + | ACCGGAUUGAACAGGCCCAG | 20 | 1440 |
| HSV1-UL19-981 | + | AUCGGCAACAACAAAGCCAG | 20 | 1367 |
| HSV1-UL19-1133 | + | GCCGCCGGACAUAGCGCCAG | 20 | 1519 |
| HSV1-UL19-1399 | + | ACGUAAAGAACUUAAAGCAG | 20 | 1785 |
| HSV1-UL19-1216 | + | GAACCCCCGCGGUGCAGCAG | 20 | 1602 |
| HSV1-UL19-1238 | + | ACCAGAUUGGCCGGGUGCAG | 20 | 1624 |
| HSV1-UL19-1341 | + | UGCCGCGUUGUCCAGGAGAG | 20 | 1727 |
| HSV1-UL19-1185 | + | GCGUCACAGUCCCACACGAG | 20 | 1571 |
| HSV1-UL19-539 | − | UUUACGCGGGGGACAAGGAG | 20 | 925 |
| HSV1-UL19-1343 | + | GCUGCCGCGUUGUCCAGGAG | 20 | 1729 |
| HSV1-UL19-1270 | + | AACAGCGCGUGGACGGGGAG | 20 | 1656 |
| HSV1-UL19-1423 | + | GGGUCGCUGGCGCAGGUGAG | 20 | 1809 |
| HSV1-UL19-1441 | + | UACAGAGCCAGUCCCUUGAG | 20 | 1827 |
| HSV1-UL19-428 | − | UCACAACGGGCGCGUGGUAG | 20 | 814 |
| HSV1-UL19-505 | − | UCCCCCUCUCCUGGACAACG | 20 | 891 |
| HSV1-UL19-576 | − | CCUCCGCAGUGCCCGCAACG | 20 | 962 |
| HSV1-UL19-556 | − | CGGGGCCUACCACCUCAACG | 20 | 942 |
| HSV1-UL19-490 | − | CACCCAGCCGCGCGCGAACG | 20 | 876 |
| HSV1-UL19-1120 | + | CCCCACAAAGAAGUCGAACG | 20 | 1506 |
| HSV1-UL19-507 | − | AGCCGUGUACCUGCGGAACG | 20 | 893 |
| HSV1-UL19-233 | − | CGGCGAGAUGGUCCUGAACG | 20 | 619 |
| HSV1-UL19-553 | − | CGGGGACCUGCUCUAUAACG | 20 | 939 |
| HSV1-UL19-393 | − | GUACGCGGCGGCAUGUAACG | 20 | 779 |
| HSV1-UL19-355 | − | CCAGCUACUGGAACAACACG | 20 | 741 |
| HSV1-UL19-1354 | + | CCUGGGCGCACCCGAACACG | 20 | 1740 |
| HSV1-UL19-1187 | + | GGGCGUCACAGUCCCACACG | 20 | 1573 |
| HSV1-UL19-1365 | + | AGUUGACGUCGGUCGACACG | 20 | 1751 |
| HSV1-UL19-1196 | + | UACAUGCCGCCGCGUACACG | 20 | 1582 |
| HSV1-UL19-449 | − | GUUCGCCGGGGCCGACCACG | 20 | 835 |
| HSV1-UL19-1279 | + | AAUUGGGCGCGUUCGCCACG | 20 | 1665 |
| HSV1-UL19-1231 | + | GGUGCGCGGGGUCCGUCACG | 20 | 1617 |
| HSV1-UL19-1008 | + | GUGUCGGCAUGCGUCAGACG | 20 | 1394 |
| HSV1-UL19-240 | − | CGUGCGAAGUCUGGACGACG | 20 | 626 |
| HSV1-UL19-437 | − | CUGCUCGGCGGCGCCCGACG | 20 | 823 |
| HSV1-UL19-310 | − | CUUUGUGGGGGUGGCCGACG | 20 | 696 |
| HSV1-UL19-225 | − | GCGCGGGCGGCCGGUCGACG | 20 | 611 |
| HSV1-UL19-441 | − | CAACAUGCGCAUAUUCGACG | 20 | 827 |
| HSV1-UL19-1091 | + | ACGGAUUGGCGGCUUCGACG | 20 | 1477 |
| HSV1-UL19-1273 | + | CGGCGAACAGCGCGUGGACG | 20 | 1659 |
| HSV1-UL19-1255 | + | GCGCAUGUUGGUGGUGGACG | 20 | 1641 |
| HSV1-UL19-1330 | + | GGUUGCCCAUGUCGGUGACG | 20 | 1716 |
| HSV1-UL19-277 | − | UUCGCUGAUGAACGUUGACG | 20 | 663 |
| HSV1-UL19-1218 | + | UCUGCAGGGUGGCGUAUACG | 20 | 1604 |
| HSV1-UL19-285 | − | CGAAGCCGCCAAUCCGUACG | 20 | 671 |
| HSV1-UL19-550 | − | GUCGCAGCGAUUUUCGUACG | 20 | 936 |
| HSV1-UL19-391 | − | GGGCCACGACCCCGUGUACG | 20 | 777 |
| HSV1-UL19-532 | − | CGCCGCCGGGGGCGUUUACG | 20 | 918 |
| HSV1-UL19-402 | − | GCGGAUGACCGGCCGCACCG | 20 | 788 |
| HSV1-UL19-411 | − | GCGGGGCCGCUGCUGCACCG | 20 | 797 |
| HSV1-UL19-394 | − | GGCAUGUAACGUGGCGACCG | 20 | 780 |
| HSV1-UL19-546 | − | UCGCGGCCACGGCCAACCCG | 20 | 932 |
| HSV1-UL19-1414 | + | CACGAGUUCGCGGCACCCCG | 20 | 1800 |
| HSV1-UL19-527 | − | CGCCGGCCCUGCAACCCCCG | 20 | 913 |
| HSV1-UL19-1217 | + | GCGGUCGAAGCGAACCCCCG | 20 | 1603 |
| HSV1-UL19-568 | − | UACAGUUUAAGCGCCCCCCG | 20 | 954 |
| HSV1-UL19-1164 | + | CCUCGGGAAGGUCUCCCCCG | 20 | 1550 |
| HSV1-UL19-1339 | + | CCAGGAGAGGGGGAGCCCCG | 20 | 1725 |
| HSV1-UL19-977 | + | UGCCGCGCUCAAACGCCCCG | 20 | 1363 |
| HSV1-UL19-1374 | + | CCCCCGGCGGCGCGUCCCCG | 20 | 1760 |
| HSV1-UL19-335 | − | GCGGCGUUCCGGGACGCCCG | 20 | 721 |
| HSV1-UL19-1108 | + | CGACCCAUCGGACCCGCCCG | 20 | 1494 |
| HSV1-UL19-322 | − | ACGUUCCCCCGGCCGGCCCG | 20 | 708 |
| HSV1-UL19-467 | − | GUACUUCAAGAUCAGUCCCG | 20 | 853 |
| HSV1-UL19-362 | − | CCUCGGGGGAGACCUUCCCG | 20 | 748 |
| HSV1-UL19-1296 | + | GUAGUUGGCCCCCAGAGCCG | 20 | 1682 |
| HSV1-UL19-397 | − | CAACACCCAGGCCCGAGCCG | 20 | 783 |
| HSV1-UL19-265 | − | ACGGUUUGCCGCGCACGCCG | 20 | 651 |
| HSV1-UL19-398 | − | GGCCCGAGCCGCGGACGCCG | 20 | 784 |
| HSV1-UL19-530 | − | CCCCCGGGGACGCGCCGCCG | 20 | 916 |
| HSV1-UL19-1359 | + | UGGUCCAUCCCUGCGCGCCG | 20 | 1745 |
| HSV1-UL19-1225 | + | CGCUGGGGCACUCCUCGCCG | 20 | 1611 |
| HSV1-UL19-448 | − | CGUCCACGCGCUGUUCGCCG | 20 | 834 |
| HSV1-UL19-1124 | + | GAUCUCCCCCGGGCCGGCCG | 20 | 1510 |
| HSV1-UL19-1097 | + | GUCUGCGGCGGGGCCGGCCG | 20 | 1483 |
| HSV1-UL19-462 | − | CGUCCGCGAGAGCGCGGCCG | 20 | 848 |
| HSV1-UL19-1284 | + | CGACAGGUCGCGCAGGGCCG | 20 | 1670 |
| HSV1-UL19-501 | − | CAAAACUUUUACCUGGGCCG | 20 | 887 |
| HSV1-UL19-1360 | + | CACACACGGCGUCCUGGCCG | 20 | 1746 |
| HSV1-UL19-1382 | + | GGGACGGGUCGCUCUGGCCG | 20 | 1768 |
| HSV1-UL19-1002 | + | CACGGAGGGCUGCGUGGCCG | 20 | 1388 |
| HSV1-UL19-1407 | + | GUCGCUGGCGGCGGUGGCCG | 20 | 1793 |
| HSV1-UL19-1066 | + | CGCGGGGAUCCGGGUGGCCG | 20 | 1452 |
| HSV1-UL19-1389 | + | CGACGCCCACGGGUUGGCCG | 20 | 1775 |
| HSV1-UL19-313 | − | UGGCCGACGUGGAGCUGCCG | 20 | 699 |
| HSV1-UL19-518 | − | UCGGGUGCGCCCAGGUGCCG | 20 | 904 |
| HSV1-UL19-1327 | + | GGGGUUGCGGACGGUUGCCG | 20 | 1713 |
| HSV1-UL19-1207 | + | CCGGUGCGGCCGGUCAUCCG | 20 | 1593 |
| HSV1-UL19-218 | − | CACGGCCACGCAGCCCUCCG | 20 | 604 |
| HSV1-UL19-1111 | + | GGGCUGCAUAAACUGCUCCG | 20 | 1497 |
| HSV1-UL19-1199 | + | GUCGUUGCGGUUGAAGUCCG | 20 | 1585 |
| HSV1-UL19-1206 | + | CCGGUCAUCCGCGGCGUCCG | 20 | 1592 |
| HSV1-UL19-459 | − | GCAGCACGUCCGCGAGAGCG | 20 | 845 |
| HSV1-UL19-1439 | + | CAGAGCCAGUCCCUUGAGCG | 20 | 1825 |
| HSV1-UL19-192 | − | GUCCUCGGGGCGUUUGAGCG | 20 | 578 |
| HSV1-UL19-386 | − | UCGCGACUGCCGGGUUAGCG | 20 | 772 |
| HSV1-UL19-1118 | + | CCACAAAGAAGUCGAACGCG | 20 | 1504 |
| HSV1-UL19-219 | − | CUGACGCAUGCCGACACGCG | 20 | 605 |
| HSV1-UL19-577 | − | GCAACGGGGAAGCCCACGCG | 20 | 963 |
| HSV1-UL19-534 | − | CCGCCGGGGGCGUUUACGCG | 20 | 920 |
| HSV1-UL19-413 | − | GGGGCCGCUGCUGCACCGCG | 20 | 799 |
| HSV1-UL19-503 | − | AAACUUUUACCUGGGCCGCG | 20 | 889 |
| HSV1-UL19-1276 | + | CGGCCCCGGCGAACAGCGCG | 20 | 1662 |
| HSV1-UL19-388 | − | GCGACUGCCGGGUUAGCGCG | 20 | 774 |
| HSV1-UL19-1436 | + | CGAAGUGGGUCUCCCGCGCG | 20 | 1822 |
| HSV1-UL19-1323 | + | CCCAGGUCCACGUUCGCGCG | 20 | 1709 |
| HSV1-UL19-1072 | + | UGGCGGGGCGGGAAGGCGCG | 20 | 1458 |
| HSV1-UL19-382 | − | UGACGCCCUGAUGCGGCGCG | 20 | 768 |
| HSV1-UL19-427 | − | CAUGUUUCACAACGGGCGCG | 20 | 813 |
| HSV1-UL19-326 | − | GAUCCAGGCCACCUGGCGCG | 20 | 712 |
| HSV1-UL19-247 | − | CCAGACGACGCGCGUGCGCG | 20 | 633 |
| HSV1-UL19-1234 | + | CCGGGUGCAGGGGGUGCGCG | 20 | 1620 |
| HSV1-UL19-410 | − | AUGGUGCCCGCCUUCUCGCG | 20 | 796 |
| HSV1-UL19-346 | − | CACCAUCGCCGCCGUUCGCG | 20 | 732 |
| HSV1-UL19-1074 | + | GCUGGCGGGGCGGGAAGGCG | 20 | 1460 |
| HSV1-UL19-1397 | + | GCAGGGGCUGAGCACCGGCG | 20 | 1783 |
| HSV1-UL19-419 | − | CCCGGAGAUCGCCCCCGGCG | 20 | 805 |
| HSV1-UL19-1101 | + | GCUGCUGCAUGUCUGCGGCG | 20 | 1487 |
| HSV1-UL19-3234 | + | CACUACCCCAAGGAAGGGCG | 20 | 7833 |
| HSV1-UL19-1426 | + | GGGCACUGCGGAGGAGGGCG | 20 | 1812 |
| HSV1-UL19-1062 | + | GAUCCGGGUGGCCGGGGGCG | 20 | 1448 |
| HSV1-UL19-493 | − | CGCGAACGUGGACCUGGGCG | 20 | 879 |
| HSV1-UL19-1141 | + | UGGUGGCGGGGGCCAUGGCG | 20 | 1527 |
| HSV1-UL19-466 | − | UGACCUACGCGCUCAUGGCG | 20 | 852 |
| HSV1-UL19-486 | − | GGCCCGGCACGAAACUGGCG | 20 | 872 |
| HSV1-UL19-1078 | + | CCCCAAAAAAACAGCUGGCG | 20 | 1464 |
| HSV1-UL19-1144 | + | GAACGGCGGCGAUGGUGGCG | 20 | 1530 |
| HSV1-UL19-1003 | + | CACGGCCACGGAGGGCUGCG | 20 | 1389 |
| HSV1-UL19-1059 | + | GACUAGGUCCCCGGCGUGCG | 20 | 1445 |
| HSV1-UL19-356 | − | CUGGAACAACACGCGGUGCG | 20 | 742 |
| HSV1-UL19-1236 | + | GGCCGGGUGCAGGGGGUGCG | 20 | 1622 |
| HSV1-UL19-560 | − | CUGCCUGGAGCGCCUGAUCG | 20 | 946 |
| HSV1-UL19-1015 | + | CAGGAGGCGCUGUUUGAUCG | 20 | 1401 |
| HSV1-UL19-569 | − | CCCGGGGUGCCGCGAACUCG | 20 | 955 |
| HSV1-UL19-360 | − | GUACGUCGUGACCUACCUCG | 20 | 746 |
| HSV1-UL19-191 | − | CAACGUCCAGGCCGUCCUCG | 20 | 577 |
| HSV1-UL19-353 | − | CUGCGCCCUGGCCCGGCUCG | 20 | 739 |
| HSV1-UL19-216 | − | GGCCGUCGAGGCCUGGCUCG | 20 | 602 |
| HSV1-UL19-1303 | + | UCUCCCCGGCCGCGCUCUCG | 20 | 1689 |
| HSV1-UL19-408 | − | UGAUGGUGCCCGCCUUCUCG | 20 | 794 |
| HSV1-UL19-368 | − | GGACCUGGUGGCGCACGUCG | 20 | 754 |
| HSV1-UL19-495 | − | CGUGGGCUACACCGCCGUCG | 20 | 881 |
| HSV1-UL19-214 | − | CCACCGCCGGGAGGCCGUCG | 20 | 600 |
| HSV1-UL19-508 | − | GUACCUGCGGAACGCGGUCG | 20 | 894 |
| HSV1-UL19-281 | − | GAACGUUGACGCGGCGGUCG | 20 | 667 |
| HSV1-UL19-1195 | + | CCGCCGCGUACACGGGGUCG | 20 | 1581 |
| HSV1-UL19-1090 | + | UGGCGGCUUCGACGGGGUCG | 20 | 1476 |
| HSV1-UL19-301 | − | CGGGCGGGUCCGAUGGGUCG | 20 | 687 |
| HSV1-UL19-451 | − | CGGCCCUGCGCGACCUGUCG | 20 | 837 |
| HSV1-UL19-544 | − | CGACCCGUCCCGGGCCUUCG | 20 | 930 |
| HSV1-UL19-1417 | + | ACGGGUCCUCCACGAGUUCG | 20 | 1803 |
| HSV1-UL19-344 | − | GCCACCAUCGCCGCCGUUCG | 20 | 730 |
| HSV1-UL19-537 | − | CGUUUACGCGGGGGACAAGG | 20 | 923 |
| HSV1-UL19-209 | − | GAGCUUUUUCCUGGGCAAGG | 20 | 595 |
| HSV1-UL19-1214 | + | GCAGCGGCCCCGCGAGAAGG | 20 | 1600 |
| HSV1-UL19-1047 | + | GGUCCAUGGCGCCCACCAGG | 20 | 1433 |
| HSV1-UL19-1290 | + | CCAGAGCCGGGGGGACCAGG | 20 | 1676 |
| HSV1-UL19-1347 | + | CGGGCUGGGCCGGCCCCAGG | 20 | 1733 |
| HSV1-UL19-1131 | + | GCCGUUCACCACGCGCCAGG | 20 | 1517 |
| HSV1-UL19-986 | + | GGGCCACCCUGGUGGCCAGG | 20 | 1372 |
| HSV1-UL19-481 | − | CUUCCUGGGCCAGCUCCAGG | 20 | 867 |
| HSV1-UL19-1030 | + | CCUCCUGCAUCUCCAGCAGG | 20 | 1416 |
| HSV1-UL19-244 | − | CCACCUGCUGGAGAUGCAGG | 20 | 630 |
| HSV1-UL19-1018 | + | CUUUCAGGAAGGACUGCAGG | 20 | 1404 |
| HSV1-UL19-1237 | + | CCAGAUUGGCCGGGUGCAGG | 20 | 1623 |
| HSV1-UL19-573 | − | CCCGUGUGGCCUGUUUCAGG | 20 | 959 |
| HSV1-UL19-1340 | + | GCCGCGUUGUCCAGGAGAGG | 20 | 1726 |
| HSV1-UL19-540 | − | UUACGCGGGGGACAAGGAGG | 20 | 926 |
| HSV1-UL19-1430 | + | CGUUGCGGGCACUGCGGAGG | 20 | 1816 |
| HSV1-UL19-1269 | + | ACAGCGCGUGGACGGGGAGG | 20 | 1655 |
| HSV1-UL19-1395 | + | CCGGCGAGGCCCCGUUGAGG | 20 | 1781 |
| HSV1-UL19-1149 | + | GUCGAACGCCCCGCGAACGG | 20 | 1535 |
| HSV1-UL19-1364 | + | GUUGACGUCGGUCGACACGG | 20 | 1750 |
| HSV1-UL19-1005 | + | CAGACGGGGCACGGCCACGG | 20 | 1391 |
| HSV1-UL19-1325 | + | GGUUGCCGUGGCCACGACGG | 20 | 1711 |
| HSV1-UL19-403 | − | CGGAUGACCGGCCGCACCGG | 20 | 789 |
| HSV1-UL19-348 | − | CGACGACCGCAACUACCCGG | 20 | 734 |
| HSV1-UL19-1413 | + | ACGAGUUCGCGGCACCCCGG | 20 | 1799 |
| HSV1-UL19-1377 | + | CCCCGCGUAAACGCCCCCGG | 20 | 1763 |
| HSV1-UL19-1373 | + | CCCCGGCGGCGCGUCCCCGG | 20 | 1759 |
| HSV1-UL19-323 | − | CGUUCCCCCGGCCGGCCCGG | 20 | 709 |
| HSV1-UL19-997 | + | AGGCCUCGACGGCCUCCCGG | 20 | 1383 |
| HSV1-UL19-1295 | + | UAGUUGGCCCCCAGAGCCGG | 20 | 1681 |
| HSV1-UL19-531 | − | CCCCGGGGACGCGCCGCCGG | 20 | 917 |
| HSV1-UL19-1224 | + | GCUGGGGCACUCCUCGCCGG | 20 | 1610 |
| HSV1-UL19-1123 | + | AUCUCCCCCGGGCCGGCCGG | 20 | 1509 |
| HSV1-UL19-1283 | + | GACAGGUCGCGCAGGGCCGG | 20 | 1669 |
| HSV1-UL19-1369 | + | CCCGGGGGUUGCAGGGCCGG | 20 | 1755 |
| HSV1-UL19-1065 | + | GCGGGGAUCCGGGUGGCCGG | 20 | 1451 |
| HSV1-UL19-314 | − | GGCCGACGUGGAGCUGCCGG | 20 | 700 |
| HSV1-UL19-330 | − | GCCCCUGGCGCUAUGUCCGG | 20 | 716 |
| HSV1-UL19-278 | − | GCUGAUGAACGUUGACGCGG | 20 | 664 |
| HSV1-UL19-392 | − | CCACGACCCCGUGUACGCGG | 20 | 778 |
| HSV1-UL19-535 | − | CGCCGGGGGCGUUUACGCGG | 20 | 921 |
| HSV1-UL19-414 | − | GGGCCGCUGCUGCACCGCGG | 20 | 800 |
| HSV1-UL19-389 | − | CGACUGCCGGGUUAGCGCGG | 20 | 775 |
| HSV1-UL19-996 | + | CCUCGACGGCCUCCCGGCGG | 20 | 1382 |
| HSV1-UL19-1408 | + | CUGUACGUCGCUGGCGGCGG | 20 | 1794 |
| HSV1-UL19-1013 | + | GCGCUGUUUGAUCGGGGCGG | 20 | 1399 |
| HSV1-UL19-487 | − | GCCCGGCACGAAACUGGCGG | 20 | 873 |
| HSV1-UL19-1409 | + | AAACUGUACGUCGCUGGCGG | 20 | 1795 |
| HSV1-UL19-1143 | + | AACGGCGGCGAUGGUGGCGG | 20 | 1529 |
| HSV1-UL19-1431 | + | CCCCGUUGCGGGCACUGCGG | 20 | 1817 |
| HSV1-UL19-1103 | + | CAGCUGCUGCAUGUCUGCGG | 20 | 1489 |
| HSV1-UL19-361 | − | UACGUCGUGACCUACCUCGG | 20 | 747 |
| HSV1-UL19-436 | − | AACGGCGCUGCUCUGCUCGG | 20 | 822 |
| HSV1-UL19-477 | − | GUUCUCGGAGCGCGCGUCGG | 20 | 863 |
| HSV1-UL19-282 | − | AACGUUGACGCGGCGGUCGG | 20 | 668 |
| HSV1-UL19-564 | − | GAUCGUGGAGACGGGUUCGG | 20 | 950 |
| HSV1-UL19-1086 | + | CAACGUUCAUCAGCGAAGGG | 20 | 1472 |
| HSV1-UL19-202 | − | CGGCCGCCUGGCCACCAGGG | 20 | 588 |
| HSV1-UL19-1219 | + | GACCACCAUGUUCUGCAGGG | 20 | 1605 |
| HSV1-UL19-1428 | + | GCGGGCACUGCGGAGGAGGG | 20 | 1814 |
| HSV1-UL19-1379 | + | GCCGUGGUCGUACAUGAGGG | 20 | 1765 |
| HSV1-UL19-297 | − | GGCAGCGCCUGGCCCACGGG | 20 | 683 |
| HSV1-UL19-404 | − | GGAUGACCGGCCGCACCGGG | 20 | 790 |
| HSV1-UL19-1412 | + | CGAGUUCGCGGCACCCCGGG | 20 | 1798 |
| HSV1-UL19-1294 | + | AGUUGGCCCCCAGAGCCGGG | 20 | 1680 |
| HSV1-UL19-213 | − | CAAGGCGGGCCACCGCCGGG | 20 | 599 |
| HSV1-UL19-1282 | + | ACAGGUCGCGCAGGGCCGGG | 20 | 1668 |
| HSV1-UL19-315 | − | GCCGACGUGGAGCUGCCGGG | 20 | 701 |
| HSV1-UL19-1069 | + | GGAAGGCGCGGGGAUCCGGG | 20 | 1455 |
| HSV1-UL19-390 | − | GACUGCCGGGUUAGCGCGGG | 20 | 776 |
| HSV1-UL19-221 | − | CGCAUGCCGACACGCGCGGG | 20 | 607 |
| HSV1-UL19-488 | − | CCCGGCACGAAACUGGCGGG | 20 | 874 |
| HSV1-UL19-980 | + | GGCAACAACAAAGCCAGGGG | 20 | 1366 |
| HSV1-UL19-1184 | + | UCACAGUCCCACACGAGGGG | 20 | 1570 |
| HSV1-UL19-1272 | + | CGAACAGCGCGUGGACGGGG | 20 | 1658 |
| HSV1-UL19-316 | − | CCGACGUGGAGCUGCCGGGG | 20 | 702 |
| HSV1-UL19-3235 | + | UACCCCAAGGAAGGGCGGGG | 20 | 7834 |
| HSV1-UL19-489 | − | CCGGCACGAAACUGGCGGGG | 20 | 875 |
| HSV1-UL19-1077 | + | CAAAAAAACAGCUGGCGGGG | 20 | 1463 |
| HSV1-UL19-1014 | + | GAGGCGCUGUUUGAUCGGGG | 20 | 1400 |
| HSV1-UL19-1363 | + | GACGUCGGUCGACACGGGGG | 20 | 1749 |
| HSV1-UL19-1064 | + | GGGAUCCGGGUGGCCGGGGG | 20 | 1450 |
| HSV1-UL19-317 | − | CGACGUGGAGCUGCCGGGGG | 20 | 703 |
| HSV1-UL19-405 | − | UGACCGGCCGCACCGGGGGG | 20 | 791 |
| HSV1-UL19-341 | − | CCGGGGCCUGGAGCUGGGGG | 20 | 727 |
| HSV1-UL19-309 | − | GUUCGACUUCUUUGUGGGGG | 20 | 695 |
| HSV1-UL19-375 | − | CUGACCGGCCCGGAGCUGGG | 20 | 761 |
| HSV1-UL19-1261 | + | GGUAUGGUCCAGAUGCUGGG | 20 | 1647 |
| HSV1-UL19-1034 | + | GCGCACGCGCGUCGUCUGGG | 20 | 1420 |
| HSV1-UL19-416 | − | CGCCACCCUGCAGAACAUGG | 20 | 802 |
| HSV1-UL19-1247 | + | CACCUGCAGCGUGAGCAUGG | 20 | 1633 |
| HSV1-UL19-464 | − | GCUGACCUACGCGCUCAUGG | 20 | 850 |
| HSV1-UL19-1192 | + | UAACCCGGCAGUCGCGAUGG | 20 | 1578 |
| HSV1-UL19-1147 | + | CCCGCGAACGGCGGCGAUGG | 20 | 1533 |
| HSV1-UL19-1309 | + | UGAACCCAAACCCGGGAUGG | 20 | 1695 |
| HSV1-UL19-1155 | + | GUGCUCGCUGCCGUGUAUGG | 20 | 1541 |
| HSV1-UL19-484 | − | GUGGCCCGGCACGAAACUGG | 20 | 870 |
| HSV1-UL19-1082 | + | GAGACAGGCGCAGCACCUGG | 20 | 1468 |
| HSV1-UL19-367 | − | GGCCGUGUACCGGGACCUGG | 20 | 753 |
| HSV1-UL19-988 | + | GGUCGCCCGGGCCACCCUGG | 20 | 1374 |
| HSV1-UL19-255 | − | CAACGUUCCCUACCCCCUGG | 20 | 641 |
| HSV1-UL19-293 | − | AGCUGUUUUUGAACGCCUGG | 20 | 679 |
| HSV1-UL19-1080 | + | UCCCCCAAAAAAACAGCUGG | 20 | 1466 |
| HSV1-UL19-340 | − | CGCCCGGGGCCUGGAGCUGG | 20 | 726 |
| HSV1-UL19-1410 | + | CUUAAACUGUACGUCGCUGG | 20 | 1796 |
| HSV1-UL19-1262 | + | UGGUAUGGUCCAGAUGCUGG | 20 | 1648 |
| HSV1-UL19-423 | − | CCUGCACCCGGCCAAUCUGG | 20 | 809 |
| HSV1-UL19-458 | − | CCUGGUCCCCCCGGCUCUGG | 20 | 844 |
| HSV1-UL19-1035 | + | CGCGCACGCGCGUCGUCUGG | 20 | 1421 |
| HSV1-UL19-252 | − | CGAGAAGCUGGUCUUUCUGG | 20 | 638 |
| HSV1-UL19-227 | − | GCAGUCCUUCCUGAAAGUGG | 20 | 613 |
| HSV1-UL19-287 | − | UCCGUACGGGGCGUACGUGG | 20 | 673 |
| HSV1-UL19-570 | − | GGGGUGCCGCGAACUCGUGG | 20 | 956 |
| HSV1-UL19-509 | − | CCUGCGGAACGCGGUCGUGG | 20 | 895 |
| HSV1-UL19-1029 | + | CCUGCAUCUCCAGCAGGUGG | 20 | 1415 |
| HSV1-UL19-1146 | + | GCGAACGGCGGCGAUGGUGG | 20 | 1532 |
| HSV1-UL19-1042 | + | GGGGUAGGGAACGUUGGUGG | 20 | 1428 |
| HSV1-UL19-1256 | + | GAAUAUGCGCAUGUUGGUGG | 20 | 1642 |
| HSV1-UL19-308 | − | CGCGUUCGACUUCUUUGUGG | 20 | 694 |
| HSV1-UL19-1094 | + | GUACGCCCCGUACGGAUUGG | 20 | 1480 |
| HSV1-UL19-1043 | + | CAGGGGGUAGGGAACGUUGG | 20 | 1429 |
| HSV1-UL19-1257 | + | GUCGAAUAUGCGCAUGUUGG | 20 | 1643 |
| HSV1-UL19-1403 | + | CUCCAGGCAGCGAUGUUUGG | 20 | 1789 |
| HSV1-UL19-271 | − | CCCGCCAGCUGUUUUUUUGG | 20 | 657 |
| HSV1-UL19-1381 | + | UCUGGCCGUGGUCGUACAUG | 20 | 1767 |
| HSV1-UL19-381 | − | GGGACUGUGACGCCCUGAUG | 20 | 767 |
| HSV1-UL19-1432 | + | CUUCCCCGUUGCGGGCACUG | 20 | 1818 |
| HSV1-UL19-506 | − | ACGCGGCAGCCGUGUACCUG | 20 | 892 |
| HSV1-UL19-292 | − | CAGCUGUUUUUGAACGCCUG | 20 | 678 |
| HSV1-UL19-513 | − | UCGUGGCGGGAAACCGCCUG | 20 | 899 |
| HSV1-UL19-339 | − | ACGCCCGGGGCCUGGAGCUG | 20 | 725 |
| HSV1-UL19-1228 | + | GGUCCGUCACGGGGUCGCUG | 20 | 1614 |
| HSV1-UL19-1263 | + | AUGGUAUGGUCCAGAUGCUG | 20 | 1649 |
| HSV1-UL19-457 | − | CCCUGGUCCCCCCGGCUCUG | 20 | 843 |
| HSV1-UL19-1036 | + | GCGCGCACGCGCGUCGUCUG | 20 | 1422 |
| HSV1-UL19-1104 | + | AAACAGCUGCUGCAUGUCUG | 20 | 1490 |
| HSV1-UL19-571 | − | GAACUCGUGGAGGACCCGUG | 20 | 957 |
| HSV1-UL19-379 | − | CGCUGCUGCCACCCCUCGUG | 20 | 765 |
| HSV1-UL19-1209 | + | GUCCAGUCCGCCCCCCGGUG | 20 | 1595 |
| HSV1-UL19-1001 | + | AGGGCUGCGUGGCCGUGGUG | 20 | 1387 |
| HSV1-UL19-307 | − | CCGCGUUCGACUUCUUUGUG | 20 | 693 |
| HSV1-UL19-3236 | − | AACCGCCCCGCCCUUCCUUG | 20 | 7835 |
| HSV1-UL19-1212 | + | UCACGUAGUAGUAAAUCUUG | 20 | 1598 |
| HSV1-UL19-1151 | + | AAAACACCGCCGGGUAGUUG | 20 | 1537 |
| HSV1-UL19-1396 | + | GCACCGGCGAGGCCCCGUUG | 20 | 1782 |
| HSV1-UL19-1434 | + | GCGCGUGGGCUUCCCCGUUG | 20 | 1820 |
| HSV1-UL19-1200 | + | GCAGCAGCUGGCCGUCGUUG | 20 | 1586 |
| HSV1-UL19-1329 | + | CCAUGUCGGUGACGGGGUUG | 20 | 1715 |
| HSV1-UL19-1250 | + | UUGUGCGUUCGGCCAUGUUG | 20 | 1636 |
| HSV1-UL19-305 | − | CCCCGCGUUCGACUUCUUUG | 20 | 691 |
| HSV1-UL19-1335 | + | CGGCCCAGGUAAAAGUUUUG | 20 | 1721 |
| HSV1-UL19-270 | − | CCCCGCCAGCUGUUUUUUUG | 20 | 656 |
| HSV1-UL19-1281 | + | GCGCAGGGCCGGGGGGAAAU | 20 | 1667 |
| HSV1-UL19-498 | − | CGCAACCCCGUCACCGACAU | 20 | 884 |
| HSV1-UL19-1109 | + | UCUGGCCUUCCGCGACCCAU | 20 | 1495 |
| HSV1-UL19-249 | − | CGCGCGGAUCUGGUGUCCAU | 20 | 635 |
| HSV1-UL19-984 | + | UUGUCCAGGUAUCGUUGCAU | 20 | 1370 |
| HSV1-UL19-1243 | + | GACCGUGUUGGCCACCAGAU | 20 | 1629 |
| HSV1-UL19-300 | − | GGCCCACGGGCGGGUCCGAU | 20 | 686 |
| HSV1-UL19-275 | − | CUGUCUCUGGAACACGCGAU | 20 | 661 |
| HSV1-UL19-1095 | + | CACGUACGCCCCGUACGGAU | 20 | 1481 |
| HSV1-UL19-237 | − | CUGGUCACGGCGCUCGUGAU | 20 | 623 |
| HSV1-UL19-1017 | + | UGCAGGAGGCGCUGUUUGAU | 20 | 1403 |
| HSV1-UL19-1061 | + | GGUGGCCGGGGGCGGGGACU | 20 | 1447 |
| HSV1-UL19-1357 | + | CCCUGCGCGCCGCGGCACCU | 20 | 1743 |
| HSV1-UL19-492 | − | CCGCGCGCGAACGUGGACCU | 20 | 878 |
| HSV1-UL19-358 | − | UCGUACGUCGUGACCUACCU | 20 | 744 |
| HSV1-UL19-500 | − | CUUCCCCAAAACUUUUACCU | 20 | 886 |
| HSV1-UL19-259 | − | CUGACGUUCGUCCUGCCCCU | 20 | 645 |
| HSV1-UL19-291 | − | GCAGCUGUUUUUGAACGCCU | 20 | 677 |
| HSV1-UL19-512 | − | GUCGUGGCGGGAAACCGCCU | 20 | 898 |
| HSV1-UL19-1202 | + | GGCGUCCGCGGCUCGGGCCU | 20 | 1588 |
| HSV1-UL19-1167 | + | GUACACGGCCAUGCACUCCU | 20 | 1553 |
| HSV1-UL19-189 | − | CGCAACGUCCAGGCCGUCCU | 20 | 575 |
| HSV1-UL19-479 | − | GCGUCGGAGGCGUACUUCCU | 20 | 865 |
| HSV1-UL19-3237 | − | UAAACCGCCCCGCCCUUCCU | 20 | 7836 |
| HSV1-UL19-207 | − | UGCGAGACGAGCUUUUUCCU | 20 | 593 |
| HSV1-UL19-1173 | + | GGUAAAGUCAUCAACCAGCU | 20 | 1559 |
| HSV1-UL19-1180 | + | UCGCAUUAGGUGAUUCAGCU | 20 | 1566 |
| HSV1-UL19-374 | − | ACCCUGACCGGCCCGGAGCU | 20 | 760 |
| HSV1-UL19-338 | − | GACGCCCGGGGCCUGGAGCU | 20 | 724 |
| HSV1-UL19-994 | + | GCAGAAGCUUCGCUUUAGCU | 20 | 1380 |
| HSV1-UL19-1229 | + | GGGUCCGUCACGGGGUCGCU | 20 | 1615 |
| HSV1-UL19-1205 | + | CAUCCGCGGCGUCCGCGGCU | 20 | 1591 |
| HSV1-UL19-1321 | + | GUCCACGUUCGCGCGCGGCU | 20 | 1707 |
| HSV1-UL19-1350 | + | CCCGAACACGGGGACGGGCU | 20 | 1736 |
| HSV1-UL19-1264 | + | GAUGGUAUGGUCCAGAUGCU | 20 | 1650 |
| HSV1-UL19-435 | − | CACAACGGCGCUGCUCUGCU | 20 | 821 |
| HSV1-UL19-456 | − | CCCCUGGUCCCCCCGGCUCU | 20 | 842 |
| HSV1-UL19-1037 | + | CGCGCGCACGCGCGUCGUCU | 20 | 1423 |
| HSV1-UL19-475 | − | GACUGAGAACGUGCUGUUCU | 20 | 861 |
| HSV1-UL19-1437 | + | GACGAGAUACUGCGCGAAGU | 20 | 1823 |
| HSV1-UL19-1299 | + | UCGGAUCGACGAAAAGUAGU | 20 | 1685 |
| HSV1-UL19-1044 | + | CACCAGGGGGUAGGGAACGU | 20 | 1430 |
| HSV1-UL19-1121 | + | CCCCCCCGGCAGCUCCACGU | 20 | 1507 |
| HSV1-UL19-1023 | + | GCCGUACGUCACCGGCACGU | 20 | 1409 |
| HSV1-UL19-241 | − | GUGCGAAGUCUGGACGACGU | 20 | 627 |
| HSV1-UL19-1368 | + | CCGGCGGAAGUAGUUGACGU | 20 | 1754 |
| HSV1-UL19-558 | − | CUGCUUUAAGUUCUUUACGU | 20 | 944 |
| HSV1-UL19-547 | − | CGCGGCCACGGCCAACCCGU | 20 | 933 |
| HSV1-UL19-1107 | + | GACCCAUCGGACCCGCCCGU | 20 | 1493 |
| HSV1-UL19-1253 | + | CGCGGUGUUGGCGCCCGCGU | 20 | 1639 |
| HSV1-UL19-1305 | + | GUACCCCGCCAUGAGCGCGU | 20 | 1691 |
| HSV1-UL19-1435 | + | GAAGUGGGUCUCCCGCGCGU | 20 | 1821 |
| HSV1-UL19-476 | − | GCUGUUCUCGGAGCGCGCGU | 20 | 862 |
| HSV1-UL19-494 | − | GCGAACGUGGACCUGGGCGU | 20 | 880 |
| HSV1-UL19-1026 | + | CACGAGCGCCGUGACCAGGU | 20 | 1412 |
| HSV1-UL19-1116 | + | GUGCAGCUCAAGAGCCAGGU | 20 | 1502 |
| HSV1-UL19-1163 | + | GGGAAGGUCUCCCCCGAGGU | 20 | 1549 |
| HSV1-UL19-279 | − | AUGAACGUUGACGCGGCGGU | 20 | 665 |
| HSV1-UL19-1390 | + | UCGCUGCGACGCCCACGGGU | 20 | 1776 |
| HSV1-UL19-1421 | + | GCUGGCGCAGGUGAGCGGGU | 20 | 1807 |
| HSV1-UL19-1046 | + | CAUGGCGCCCACCAGGGGGU | 20 | 1432 |
| HSV1-UL19-342 | − | CGGGGCCUGGAGCUGGGGGU | 20 | 728 |
| HSV1-UL19-256 | − | AACGUUCCCUACCCCCUGGU | 20 | 642 |
| HSV1-UL19-978 | + | CAGCACGUGCAGCAUCUGGU | 20 | 1364 |
| HSV1-UL19-1278 | + | GGGCGCGUUCGCCACGUGGU | 20 | 1664 |
| HSV1-UL19-1394 | + | CGAGGCCCCGUUGAGGUGGU | 20 | 1780 |
| HSV1-UL19-1333 | + | UUGGGGAAGGUUGCCCAUGU | 20 | 1719 |
| HSV1-UL19-1258 | + | CCCGUCGAAUAUGCGCAUGU | 20 | 1644 |
| HSV1-UL19-1402 | + | GCGAUGUUUGGCGGCGAUGU | 20 | 1788 |
| HSV1-UL19-1314 | + | CAGUCACAAAGCGGUCCUGU | 20 | 1700 |
| HSV1-UL19-3213 | + | GGACGUUGCGAGCGAGCUGU | 20 | 7812 |
| HSV1-UL19-1300 | + | CGUGCUGCACGACGGGCUGU | 20 | 1686 |
| HSV1-UL19-1244 | + | AAACAUGGCGUUGACCGUGU | 20 | 1630 |
| HSV1-UL19-1011 | + | GACCGGCCGCCCGCGCGUGU | 20 | 1397 |
| HSV1-UL19-380 | − | GCUGCUGCCACCCCUCGUGU | 20 | 766 |
| HSV1-UL19-1254 | + | GUUGGUGGUGGACGCGGUGU | 20 | 1640 |
| HSV1-UL19-1115 | + | AAGAGCCAGGUUGGCGUUGU | 20 | 1501 |
| HSV1-UL19-1249 | + | UGUGCGUUCGGCCAUGUUGU | 20 | 1635 |
| HSV1-UL19-306 | − | CCCGCGUUCGACUUCUUUGU | 20 | 692 |
| HSV1-UL19-1280 | + | CGCAGGGCCGGGGGGAAAUU | 20 | 1666 |
| HSV1-UL19-1181 | + | GCAGCGCCGGGUCUCGCAUU | 20 | 1567 |
| HSV1-UL19-3238 | − | AAACCGCCCCGCCCUUCCUU | 20 | 7837 |
| HSV1-UL19-1022 | + | CGUCACCGGCACGUCGGCUU | 20 | 1408 |
| HSV1-UL19-1251 | + | GAGCAGCGCCGUUGUGCGUU | 20 | 1637 |
| HSV1-UL19-563 | − | CCUGAUCGUGGAGACGGGUU | 20 | 949 |
| HSV1-UL19-472 | − | ACGGGCCUCCAUCCCGGGUU | 20 | 858 |
| HSV1-UL19-1404 | + | GCGCUCCAGGCAGCGAUGUU | 20 | 1790 |
| HSV1-UL19-515 | − | GCCCAGCCCGUCCCCGUGUU | 20 | 901 |
| HSV1-UL19-1268 | + | GGUAAAAAUAGUCGCCAUUU | 20 | 1654 |
| HSV1-UL19-1337 | + | CGCGGCCCAGGUAAAAGUUU | 20 | 1723 |
| HSV1-UL19-473 | − | CGGGCCUCCAUCCCGGGUUU | 20 | 859 |
| HSV1-UL19-1336 | + | GCGGCCCAGGUAAAAGUUUU | 20 | 1722 |
| HSV1-UL19-268 | − | CGCCCCGCCAGCUGUUUUUU | 20 | 654 |
| HSV1-UL19-269 | − | GCCCCGCCAGCUGUUUUUUU | 20 | 655 |

1. A CRISPR/Cas system, comprising:
a gRNA molecule comprising a targeting domain which is complementary with a target sequence of a herpes simplex virus (HSV) viral gene selected from the group consisting of a UL19 gene, a UL30 gene, a UL48 gene, and a UL54 gene; and
a Cas9 molecule.

2. The system of claim 1, wherein said system is configured to form a double strand break or a single strand break within 500 bp, 450 bp, 400 bp, 350 bp, 300 bp, 250 bp, 200 bp, 150 bp, 100 bp, 50 bp, 25 bp, or 10 bp of a target position within said HSV viral gene, thereby altering said HSV viral gene.

3. The system of claim 1, wherein said Cas9 molecule is an enzymatically active Cas9 (eaCas9) molecule.

4. The system of claim 3, wherein ii) said eaCas9 molecule comprises a nickase molecule, (ii) said eaCas9 molecule comprises N-terminal RuvC-like domain cleavage activity but has no, or no significant, HNH-like domain cleavage activity or (iii) said eaCas9 molecule comprises HNH-like domain cleavage activity but has no, or no significant, N-terminal RuvC-like domain cleavage activity.

5. (canceled)

6. The system of claim 4, wherein (i) said eaCas9 molecule is an HNH-like domain nickase, (ii) said eaCas9 molecule comprises a mutation at D10 and/or (iii) said eaCas9 molecule is an N-terminal RuvC-like domain nickase.

7-9. (canceled)

10. The system of claim 1, wherein said Cas9 molecule is an S. aureus Cas9 molecule, an S. pyogenes Cas9 molecule, or a N. meningitidis Cas9 molecule.

11. (canceled)

12. The system of claim 1, wherein said targeting domain is configured to target a coding region or a non-coding region of said HSV viral gene, wherein said non-coding region comprises a promoter region, an enhancer region, an intron, the 3′ UTR, the 5′ UTR, or a polyadenylation signal region of said HSV viral gene; and said coding region comprises an exon of said HSV viral gene.

13. The system of claim 1, wherein said targeting domain comprises or consists of a nucleotide sequence that is the same as, or differs by no more than 3 nucleotides from, a targeting domain sequence selected from the targeting domain sequences disclosed in Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C and Table 27.

14. The system of claim 1, wherein (i) said gRNA is a modular gRNA molecule or a chimeric gRNA molecule, and/or (ii) said targeting domain has a length of 16, 17, 18, 19, 20, 21, 22, 23, 24, 25, or 26 nucleotides.

15. (canceled)

16. The system of claim 1, wherein said gRNA molecule comprises from 5′ to 3′:
a targeting domain;
a first complementarity domain;
a linking domain;
a second complementarity domain;
a proximal domain; and
a tail domain.

17. The system of claim 16, wherein (i) said linking domain is no more than 25 nucleotides in length and/or (ii) said proximal and tail domain, taken together, are at least 20, at least 25, at least 30, or at least 40 nucleotides in length.

18. (canceled)

19. A cell transfected with the CRISPR/Cas system of claim 1.

20. A gRNA molecule comprising a targeting domain which is complementary with a target sequence of a HSV viral gene selected from the group consisting of a UL19 gene, a UL30 gene, a UL48 gene, and a UL54 gene.

21. The gRNA molecule of claim 20, wherein said targeting domain comprises or consists of a nucleotide sequence that is the same as, or differs by no more than 3 nucleotides from, a targeting domain sequence selected from the targeting domain sequences disclosed Tables 1A-1G, Tables 2A-2G, Tables 3A-3G, Tables 4A-4F, Tables 5A-5E, Tables 6A-6G, Tables 7A-7D, Tables 8A-8E, Tables 9A-9G, Tables 10A-10C, Tables 11A-11E, Tables 12A-12G, Tables 13A-13C, Tables 14A-14E, Tables 15A-15G, Tables 16A-16C and Table 27.

22. A composition comprising the gRNA molecule of claim 20.

23. (canceled)

24. A nucleic acid composition comprising: (a) a first nucleotide sequence that encodes the gRNA molecule of claim 20.

25. The nucleic acid composition of claim 24, further comprising: (b) a second nucleotide sequence that encodes a Cas9 molecule and/or (c) a third nucleotide sequence that encodes a second gRNA molecule comprising a targeting domain that is complementary with a second target sequence of said HSV viral gene.

26. The nucleic acid of claim 25, ii) wherein said Cas9 molecule is an eaCas9 molecule, (ii) wherein said Cas9 molecule is an S. aureus Cas9 molecule, an S. pyogenes Cas9 molecule, or a N. meningitidis Cas9 molecule and/or (iii) wherein (a) and (b) are present on one nucleic acid molecule or (a) is present on a first nucleic acid molecule and (b) is present on a second nucleic acid molecule.

27-29. (canceled)

30. A cell transfected with the nucleic acid composition of claim 24.

31. A method of altering a HSV viral gene selected from the group consisting of a UL19 gene, a UL30 gene, a UL48 gene, and a UL54 gene in a cell, comprising administering to said cell:
(i) a CRISPR/Cas system comprising: (a) a gRNA molecule comprising a targeting domain which is complementary with a target domain sequence of said HSV viral gene and (b) a Cas9 molecule; or
(ii) a nucleic acid composition that comprises: (a) a first nucleotide sequence encoding a gRNA molecule comprising a targeting domain that is complementary with a target sequence of said HSV viral gene and (b) a second nucleotide sequence encoding a Cas9 molecule.

32-36. (canceled)
